SEQUENCE LISTING (91 sequences)

SEQ ID NO: 1 - protein, 133 aa, Homo sapiens [sequence omitted]

SEQ ID NO: 2 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 3 - protein, 133 aa, Homo sapiens
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 4 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 5 - protein, 133 aa, Homo sapiens
APTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 6 - protein, 133 aa, Homo sapiens
APTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - RNA, 822 nt, Homo sapiens
  source 1..822: mol_type = genomic RNA
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 8 - protein, 584 aa, Homo sapiens
AHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 9 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 11 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 12 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 13 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 14 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaac

SEQ ID NO: 15 - DNA, 749 bp, synthetic construct
  source 1..749: mol_type = other DNA
ttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcggccacc

SEQ ID NO: 16 - protein, 8 aa, synthetic construct
  VARIANT 2: note = V or I
  VARIANT 4: note = Any amino acid
DXEXNPGP

SEQ ID NO: 17 - protein, 118 aa, synthetic construct
EVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSS

SEQ ID NO: 18 - protein, 884 aa, synthetic construct
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGKPIPNPLLGLDST

SEQ ID NO: 19 - protein, 884 aa, synthetic construct
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGKPIPNPLLGLDST

SEQ ID NO: 20 - protein, 883 aa, synthetic construct
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGKPIPNPLLGLDS

SEQ ID NO: 21 - RNA, 2652 nt, synthetic construct
  source 1..2652: mol_type = other RNA
atgcctctgttactgctgctcccacttctgtgggccggcgctctggctgaggtgcagcttgtgcagaccggcggaggtctgtcacagttcggcgaatccctaaggctctcttgcgccgtgtctggtttcaacgtgagcaacaactatatgagttgggttcgccaggctccaggcaagggtctggagtgggtgtccattatctattccggcggtggcacacattacgccgactcagtcaagggtagattcactatcagtagagacaactcaaagaacactcttttcctacaaatgaacagcttgcgggcagaggatacggccgtgtattattgtgcccgagctgtcccagtgcctcacggaacagatatttggggacaaggtacaatggtgaccgtctcttccggcggaggaggctctggaggcggaggatctggcggaggcggcagcgcccataagagcgaggtagcccatagattcaaggaccttggcgaagagaacttcaaggccttagttctcatcgccttcgcccaatacttgcagcagtgtccattcgaagatcacgttaagcttgtgaacgaggtgaccgaattcgctaagacgtgcgtggccgacgagtccgctgagaattgtgataagtcactgcacactttgttcggcgacaagctgtgcaccgtcgccaccctgcgcgaaacatacggcgaaatggcggactgttgtgctaagcaggagccagaacggaacgagtgtttcctgcagcataaggacgacaatccaaacctccctaggttggtgcgcccagaagtggacgtcatgtgtaccgctttccacgataacgaggagacattccttaagaagtatctgtacgagattgcacggcggcacccttacttctacgcccctgagctcctgttcttcgcaaagcggtataaggctgcattcaccgagtgttgccaggcggcagacaaggccgcctgcttgctccctaagcttgacgaacttagggacgagggaaaggctagtagcgccaagcagagactcaagtgtgcatcgctacagaagttcggagagagagctttcaaggcgtgggctgtcgcgcgcctgtcccagaggttcccaaaggccgaattcgccgaggtgagcaagttagtgacagatctcaccaaggtgcacactgagtgttgccacggcgacttattggagtgtgcagacgatcgggctgacttggcaaagtacatctgtgagaaccaggactcaatttccagtaagctgaaggagtgttgcgagaagccactcttggagaagtcccattgtatagccgaagtggagaacgacgagatgccggctgacctgccgtcactggcagctgatttcgtagagtcgaaggacgtgtgtaagaactacgctgaagctaaggacgtattcctgggaatgttcctttacgagtacgcccgacggcatccagactactcagtagtgctgcttctgagactggccaagacttacgagactaccctggagaagtgttgtgcagcggccgatccacacgagtgttacgccaaggtgttcgacgaattcaagcctctggtggaggagccgcagaacctgatcaagcagaattgcgaactgttcgagcagttgggagaatataagttccagaacgcactgctagtgcggtataccaagaaggtcccacaggtatccactccgacactggtggaagtgtccaggaacctcggcaaggtgggaagcaagtgctgtaagcacccggaggctaagcggatgccgtgcgccgaagactacctcagcgtggtccttaatcagctgtgcgtgctgcacgagaagaccccagtaagtgaccgggttacgaagtgctgtactgaatcacttgtgaacagaagaccttgtttctccgccctcgaggtggacgaaacgtacgttccgaaggaattcaacgccgaaacattcaccttccacgctgacatttgtacgctttccgagaaggagagacaaatcaagaagcagactgccctggtggaattagtgaagcacaagccgaaggccacaaaggagcagctgaaggcagttatggacgacttcgctgcattcgtagagaagtgctgcaaggcagacgacaaggaaacttgcttcgcagaggagggaaagaagctcgttgctgcgtctcaggcggctctgggactgggaggaggatccgctcctacaagcagttccacaaagaagacccagctccagcttgaacatctgcttctcgacctgcaaatgatcctcaacggtatcaataattacaagaatcctaagttgactcgcatgctgactttcaagttctatatgccaaagaaggccactgagttgaagcacctgcagtgcctcgaggaagaactgaagcctctggaggaagtcttaaacctggcacaaagcaagaacttccatctgaggcctagggacctcatttccaatatcaacgttattgtgctggagctcaagggtagcgagacgacattcatgtgcgagtacgcagacgagacagcgactatcgtcgagttcctaaataggtggataacgttctgccagtcaatcatctccactctgaccggcaagccgatccctaacccgttgcttggattggattctaca

SEQ ID NO: 22 - RNA, 2652 nt, synthetic construct
  source 1..2652: mol_type = other RNA
atgcctctgctgctcttactgccgctgctttgggcaggagctctggccgaggtccagctagtgcagacaggcggtggactcagccagttcggtgagtcactgcgtctatcctgcgcggttagtggcttcaacgtgtccaacaattatatgagttgggtgagacaagcacctggcaagggccttgagtgggtgtccattatatatagcggtggcggcacccactacgctgactcggtgaagggtcgattcacaattagcagagacaattccaagaatactctgttcctgcagatgaactctctcagggcggaggacaccgccgtgtactactgtgcccgtgcggtgccggtccctcacggtacagacatctggggccagggtaccatggtcaccgtgtcatccggaggaggcggttctggtggaggaggaagtggaggcggcggcagtgcacataagagcgaggtggcccatagattcaaggatttaggagaggagaacttcaaggcactggtcttgatcgccttcgcacaatatcttcagcagtgtccgttcgaagatcacgtgaagctcgtcaacgaggtgactgagttcgccaagacctgcgtggcagacgagtcggctgagaattgtgataagagtctgcacaccctcttcggagataagctgtgtaccgtggccactctgagggagacatacggcgagatggctgactgttgtgcaaagcaggagccagagaggaacgagtgtttcttgcagcataaggacgacaacccaaatctgcctcgtctggttagacctgaagtggacgtaatgtgcaccgctttccacgacaacgaggagacgttcctgaagaagtatctgtacgagattgctagaaggcacccatacttctacgcaccagagctgttgttcttcgctaagagatacaaggccgctttcaccgagtgctgccaagcagcagacaaggctgcttgcctgctccctaagctggacgagctgagagacgagggtaaggcttcatcagccaagcaaagattgaagtgtgcatcactgcagaagttcggcgaaagagcattcaaggcttgggccgtcgccaggctgtctcagaggttcccgaaggccgagttcgctgaagtgtctaagttagtgaccgatctaacaaaggtgcacacagagtgttgccacggagatctgcttgagtgcgccgacgatcgagccgatcttgcaaagtacatctgtgagaaccaggattccatctcctctaagttgaaggagtgctgtgagaagccacttctcgagaagtctcactgcatcgcagaagtcgagaacgacgaaatgccagctgacctgccgagcttggccgcagatttcgtcgagagtaaggacgtttgcaagaattacgctgaggctaaggacgtcttcctgggcatgttcttgtacgaatacgccaggaggcacccggattactcagtggtacttttactgcgactggctaagacctacgaaacgacactggagaagtgctgcgcagccgccgatcctcacgagtgctacgctaaggttttcgacgagttcaagccgctcgtggaagaacctcagaacttaatcaagcagaattgtgagctattcgaacagctcggagagtataagttccagaacgccctcctggtgcggtacacaaagaaggttccacaggtcagcaccccgaccctcgttgaggtgagccgcaacctcggcaaggttggctctaagtgttgtaagcatccggaagcgaagcgcatgccttgtgcagaagattatctttcagttgtgttgaaccagctctgtgtcctgcacgagaagaccccagtttccgaccgcgttaccaagtgttgcacagaaagcttggtcaaccggaggccttgcttcagcgcattggaggtagacgaaacctacgtccctaaggaattcaacgccgaaacattcacgttccacgcggacatttgcacactctccgagaaggaacgccagattaagaagcagactgccttggttgagctggtaaagcacaagcctaaggcgactaaggagcagctaaaggcagttatggacgacttcgctgcattcgttgagaagtgctgtaaggccgacgataaggaaacctgcttcgccgaggaaggaaagaagcttgttgccgctagccaggccgcactgggattgggtggaggcagcgcaccaacttcctctagcaccaagaagactcaattacaactggaacatctcttactaacactccaaatgatactgaacggcataaacaactataagaacccgaagttgaccagaatgttaaccttcaagttctacatgcctaagaaggctacggaactgaagcaccttcagtgcctagaggaggagctcaagcctctcgaggaggttctgaacctcgcacagagtaagaatttccacctgcgcccaagggacctgatttccaatattaacgtgatcgtccttgaacttaagggcagcgaaaccactttcatgtgcgaatacgcggacgaaactgctaccatagttgagttccttaaccgctggatcacattctgccagtcaatcatctctacattgaccggaaagccgattccgaacccactcctgggacttgatagcaca

SEQ ID NO: 23 - RNA, 2652 nt, synthetic construct
  source 1..2652: mol_type = other RNA
atgcctctgctcctgcttcttcctctgctctgggctggcgccctagccgaggtgcagctggtgcagaccggcggcggcctgagccagttcggcgagagcctgagactgagctgcgccgtgagcggcttcaacgtgagcaacaactacatgagctgggtgagacaggcccctggcaagggcctggagtgggtgagcatcatctacagtggcggaggtacccactacgccgacagcgtgaagggcagattcaccatcagcagagacaacagcaagaacaccctgttcctgcagatgaacagcctcagggccgaggacaccgccgtgtactactgcgccagagccgtgcctgtgcctcacggcaccgacatctggggccagggcaccatggtgaccgtgagtagcggtggaggcggaagcggaggcggtggctctggaggtggcggcagcgcccacaagagcgaggtggcccacagattcaaggacctgggcgaggagaacttcaaggccctggtgctgatcgccttcgcccagtacctgcagcagtgccctttcgaggaccacgtgaagctggtgaacgaggtgaccgagttcgccaagacctgcgtggccgacgagagcgccgagaactgcgacaagagcctgcacacactcttcggcgacaagctgtgcaccgtggccaccctgagagagacttacggcgagatggccgactgctgcgccaagcaggaaccagagaggaacgagtgcttccttcagcacaaggacgacaaccctaacctgcctagactggttcggcctgaggtggacgtgatgtgcactgccttccacgacaacgaggagacgttcctgaagaagtacctgtacgagatcgccagaagacacccttacttctacgcccctgagcttctgttcttcgcaaagagatacaaggccgccttcaccgagtgctgccaggccgccgacaaggctgcctgcctcctgccaaagctggacgagctgagagacgagggcaaggccagctctgcgaagcagaggctgaagtgcgccagcctgcagaagttcggagaaagagctttcaaggcctgggccgtggcccgcttgtctcaaagattccctaaggcggagttcgcagaagtgagcaagctggtcaccgacctgaccaaggtgcatactgagtgttgccacggcgacctgctggagtgcgccgacgacagagccgacctggccaagtacatctgcgagaaccaggactctattagtagcaagttgaaggagtgttgcgagaagccgctactcgagaagagtcactgcattgcggaggttgagaacgacgagatgcctgccgacctcccaagcctggcagccgatttcgtggagagcaaggacgtgtgcaagaattacgctgaggctaaggacgttttcctgggcatgttcttatacgaatacgctagacggcacccagactacagcgtggtcctcctcctgcggcttgcaaagacgtacgaaactaccctggagaagtgctgcgcggctgctgaccctcacgagtgctacgccaaggtgttcgacgagttcaagcctctcgtggaggagcctcagaacctgatcaagcagaattgcgagctgttcgagcagttaggtgagtacaagttccagaacgccctgcttgtgcgctacaccaagaaggtgcctcaggtttccacccctaccctggtagaggtgtccagaaacctgggcaaggtgggcagcaagtgttgcaagcatccagaagctaagagaatgccttgcgccgaagattacctgtctgtcgtgctgaaccagctgtgcgtgctgcacgagaagacccctgtgagcgacagagtgaccaagtgctgtacagaatccctagtaaatagaagaccttgcttctccgccctagaggtggacgaaacctacgtgcctaaggagttcaacgccgagactttcacgttccacgcggatatttgtacactcagcgagaaggagagacagatcaagaagcagaccgcacttgtggagctcgtcaagcacaagccaaaggccaccaaggagcaactcaaggccgtgatggacgacttcgcagccttcgtcgagaagtgttgtaaggctgacgataaggaaacctgcttcgctgaggaaggcaagaagcttgtggccgccagtcaggctgctcttggtcttggcggcggatcagcacctaccagcagctctacaaagaagacgcagctgcaactggagcacttgctgcttgatctgcaaatgatcctgaacggcatcaacaattacaagaaccctaagctgaccagaatgctgaccttcaagttctacatgcctaagaaggccacagagctgaagcatttacagtgcctggaggaggagcttaagccacttgaagaggtgctcaatctggcccagtctaagaacttccacctcaggcctcgcgatctgattagcgacatcaacgtaatcgtgctggaacttaagggatctgaaactacgttcatgtgcgagtacgccgacgaaacagccaccatcgtggagttcctgaaccgatggatcaccttctgccagagtattatttcaaccctcaccggcaagcctatccctaacccactgctcggactcgacagcacc

SEQ ID NO: 24 - protein, 6 aa, synthetic construct
CHYSEL

SEQ ID NO: 25 - RNA, 2238 nt, synthetic construct
  source 1..2238: mol_type = other RNA
  modified_base 2: mod_base = OTHER; note = N1-methylpseudouracil
  modified_base 7: mod_base = OTHER; note = N1-methylpseudouracil
  modified_base 11: mod_base = OTHER; note = N1-methylpseudouracil
  modified_base 16: mod_base = OTHER; note = N1-methylpseudouracil
  modified_base 17: mod_base = OTHER; note = N1-methylpseudouracil
  modified_base 20: mod_base = OTHER; note = N1-methylpseudouracil
  modified_base 26: mod_base = OTHER; note = N1-methylpseudouracil
  modified_base 29: mod_base = OTHER; note = N1-methylpseudouracil
  modified_base 31: mod_base = OTHER; note = N1-methylpseudouracil
  modified_base 32: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 38: mod_base = OTHER; note = N1-methylpseudouracil
  modified_base 49: mod_base = OTHER; note = N1-methylpseudouracil
  modified_base 62: mod_base = OTHER; note = N1-methylpseudouracil
  modified_base 64: mod_base = OTHER; note = N1-methylpseudouracil
  modified_base 65: mod_base = OTHER; note = N1-methylpseudouracil
  modified_base 92: mod_base = OTHER; note = N1-methylpseudouracil
  modified_base 103: mod_base = OTHER; note = N1-methylpseudouracil
  modified_base 104: mod_base = OTHER; note = N1-methylpseudouracil
  modified_base 113: mod_base = OTHER; note = N1-methylpseudouracil
  modified_base 127: mod_base = OTHER; note = N1-methylpseudouracil
  modified_base 128: mod_base = OTHER; note = N1-methylpseudouracil
  modified_base 137: mod_base = OTHER; note = N1-methylpseudouracil
  modified_base 140: mod_base = OTHER; note = N1-methylpseudouracil
  modified_base 143: mod_base = OTHER; note = N1-methylpseudouracil
  modified_base 146: mod_base = OTHER; note = N1-methylpseudouracil
  modified_base 151: mod_base = OTHER; note = N1-methylpseudouracil
  modified_base 152: mod_base = OTHER; note = N1-methylpseudouracil
  modified_base 160: mod_base = OTHER; note = N1-methylpseudouracil
  modified_base 164: mod_base = OTHER; note = N1-methylpseudouracil
  modified_base 172: mod_base = OTHER; note = N1-methylpseudouracil
  modified_base 177: mod_base = OTHER; note = N1-methylpseudouracil
  modified_base 178: mod_base = OTHER; note = N1-methylpseudouracil
  modified_base 179: mod_base = OTHER; note = N1-methylpseudouracil
  modified_base 191: mod_base = OTHER; note = N1-methylpseudouracil
  modified_base 197: mod_base = OTHER; note = N1-methylpseudouracil
  modified_base 200: mod_base = OTHER; note = N1-methylpseudouracil
  modified_base 209: mod_base = OTHER; note = N1-methylpseudouracil
  modified_base 217: mod_base = OTHER; note = N1-methylpseudouracil
  modified_base 218: mod_base = OTHER; note = N1-methylpseudouracil
  modified_base 229: mod_base = OTHER; note = N1-methylpseudouracil
  modified_base 233: mod_base = OTHER; note = N1-methylpseudouracil
  modified_base 256: mod_base = OTHER; note = N1-methylpseudouracil
  modified_base 269: mod_base = OTHER; note = N1-methylpseudouracil
  modified_base 278: mod_base = OTHER; note = N1-methylpseudouracil
  modified_base 280: mod_base = OTHER; note = N1-methylpseudouracil
  modified_base 281: mod_base = OTHER; note = N1-methylpseudouracil
  modified_base 293: mod_base = OTHER; note = N1-methylpseudouracil
  modified_base 295: mod_base = OTHER; note = N1-methylpseudouracil
  modified_base 302: mod_base = OTHER; note = N1-methylpseudouracil
  modified_base 311: mod_base = OTHER; note = N1-methylpseudouracil
  modified_base 321: mod_base = OTHER; note = N1-methylpseudouracil
  modified_base 322: mod_base = OTHER; note = N1-methylpseudouracil
  modified_base 332: mod_base = OTHER; note = N1-methylpseudouracil
  modified_base 340: mod_base = OTHER; note = N1-methylpseudouracil
  modified_base 343: mod_base = OTHER; note = N1-methylpseudouracil
  modified_base 373: mod_base = OTHER; note = N1-methylpseudouracil
  modified_base 376: mod_base = OTHER; note = N1-methylpseudouracil
  modified_base 377: mod_base = OTHER; note = N1-methylpseudouracil
  modified_base 380: mod_base = OTHER; note = N1-methylpseudouracil
  modified_base 402: mod_base = OTHER; note = N1-methylpseudouracil
  modified_base 407: mod_base = OTHER; note = N1-methylpseudouracil
  modified_base 411: mod_base = OTHER; note = N1-methylpseudouracil
  modified_base 416: mod_base = OTHER; note = N1-methylpseudouracil
  modified_base 419: mod_base = OTHER; note = N1-methylpseudouracil
  modified_base 426: mod_base = OTHER; note = N1-methylpseudouracil
  modified_base 431: mod_base = OTHER; note = N1-methylpseudouracil
  modified_base 437: mod_base = OTHER; note = N1-methylpseudouracil
  modified_base 440: mod_base = OTHER; note = N1-methylpseudouracil
  modified_base 442: mod_base = OTHER; note = N1-methylpseudouracil
  modified_base 444: mod_base = OTHER; note = N1-methylpseudouracil
  modified_base 451: mod_base = OTHER; note = N1-methylpseudouracil
  modified_base 452: mod_base = OTHER; note = N1-methylpseudouracil
  modified_base 472: mod_base = OTHER; note = N1-methylpseudouracil
  modified_base 473: mod_base = OTHER; note = N1-methylpseudouracil
  modified_base 476: mod_base = OTHER; note = N1-methylpseudouracil
  modified_base 484: mod_base = OTHER; note = N1-methylpseudouracil
  modified_base 488: mod_base = OTHER; note = N1-methylpseudouracil
  modified_base 490: mod_base = OTHER; note = N1-methylpseudouracil
  modified_base 497: mod_base = OTHER; note = N1-methylpseudouracil
  modified_base 513: mod_base = OTHER; note = N1-methylpseudouracil
  modified_base 514: mod_base = OTHER; note = N1-methylpseudouracil
  modified_base 517: mod_base = OTHER; note = N1-methylpseudouracil
  modified_base 518: mod_base = OTHER; note = N1-methylpseudouracil
  modified_base 520: mod_base = OTHER; note = N1-methylpseudouracil
  modified_base 528: mod_base = OTHER; note = N1-methylpseudouracil
  modified_base 532: mod_base = OTHER; note = N1-methylpseudouracil
  modified_base 533: mod_base = OTHER; note = N1-methylpseudouracil
  modified_base 536: mod_base = OTHER; note = N1-methylpseudouracil
  modified_base 538: mod_base = OTHER; note = N1-methylpseudouracil
  modified_base 539: mod_base = OTHER; note = N1-methylpseudouracil
  modified_base 541: mod_base = OTHER; note = N1-methylpseudouracil
  modified_base 542: mod_base = OTHER; note = N1-methylpseudouracil
  modified_base 553: mod_base = OTHER; note = N1-methylpseudouracil
  modified_base 565: mod_base = OTHER; note = N1-methylpseudouracil
  modified_base 566: mod_base = OTHER; note = N1-methylpseudouracil
  modified_base 574: mod_base = OTHER; note = N1-methylpseudouracil
  modified_base 577: mod_base = OTHER; note = N1-methylpseudouracil
  modified_base 591: mod_base = OTHER; note = N1-methylpseudouracil
  modified_base 601: mod_base = OTHER; note = N1-methylpseudouracil
  modified_base 605: mod_base = OTHER; note = N1-methylpseudouracil
  modified_base 608: mod_base = OTHER; note = N1-methylpseudouracil
  modified_base 612: mod_base = OTHER; note = N1-methylpseudouracil
  modified_base 617: mod_base = OTHER; note = N1-methylpseudouracil
  modified_base 626: mod_base = OTHER; note = N1-methylpseudouracil
  modified_base 646: mod_base = OTHER; note = N1-methylpseudouracil
  modified_base 654: mod_base = OTHER; note = N1-methylpseudouracil
  modified_base 665: mod_base = OTHER; note = N1-methylpseudouracil
  modified_base 670: mod_base = OTHER; note = N1-methylpseudouracil
  modified_base 680: mod_base = OTHER; note = N1-methylpseudouracil
  modified_base 688: mod_base = OTHER; note = N1-methylpseudouracil
  modified_base 689: mod_base = OTHER; note = N1-methylpseudouracil
  modified_base 702: mod_base = OTHER; note = N1-methylpseudouracil
  modified_base 703: mod_base = OTHER; note = N1-methylpseudouracil
  modified_base 704: mod_base = OTHER; note = N1-methylpseudouracil
  modified_base 712: mod_base = OTHER; note = N1-methylpseudouracil
  modified_base 719: mod_base = OTHER; note = N1-methylpseudouracil
  modified_base 723: mod_base = OTHER; note = N1-methylpseudouracil
  modified_base 727: mod_base = OTHER; note = N1-methylpseudouracil
  modified_base 728: mod_base = OTHER; note = N1-methylpseudouracil
  modified_base 739: mod_base = OTHER; note = N1-methylpseudouracil
  modified_base 740: mod_base = OTHER; note = N1-methylpseudouracil
  modified_base 744: mod_base = OTHER; note = N1-methylpseudouracil
  modified_base 754: mod_base = OTHER; note = N1-methylpseudouracil
  modified_base 755: mod_base = OTHER; note = N1-methylpseudouracil
  modified_base 759: mod_base = OTHER; note = N1-methylpseudouracil
  modified_base 764: mod_base = OTHER; note = N1-methylpseudouracil
  modified_base 773: mod_base = OTHER; note = N1-methylpseudouracil
  modified_base 776: mod_base = OTHER; note = N1-methylpseudouracil
  modified_base 780: mod_base = OTHER; note = N1-methylpseudouracil
  modified_base 785: mod_base = OTHER; note = N1-methylpseudouracil
  modified_base 794: mod_base = OTHER; note = N1-methylpseudouracil
  modified_base 798: mod_base = OTHER; note = N1-methylpseudouracil
  modified_base 805: mod_base = OTHER; note = N1-methylpseudouracil
  modified_base 808: mod_base = OTHER; note = N1-methylpseudouracil
  modified_base 810: mod_base = OTHER; note = N1-methylpseudouracil
  modified_base 821: mod_base = OTHER; note = N1-methylpseudouracil
  modified_base 824: mod_base = OTHER; note = N1-methylpseudouracil
  modified_base 829: mod_base = OTHER; note = N1-methylpseudouracil
  modified_base 851: mod_base = OTHER; note = N1-methylpseudouracil
  modified_base 859: mod_base = OTHER; note = N1-methylpseudouracil
  modified_base 863: mod_base = OTHER; note = N1-methylpseudouracil
  modified_base 865: mod_base = OTHER; note = N1-methylpseudouracil
  modified_base 884: mod_base = OTHER; note = N1-methylpseudouracil
  modified_base 896: mod_base = OTHER; note = N1-methylpseudouracil
  modified_base 904: mod_base = OTHER; note = N1-methylpseudouracil
  modified_base 906: mod_base = OTHER; note = N1-methylpseudouracil
  modified_base 907: mod_base = OTHER; note = N1-methylpseudouracil
  modified_base 909: mod_base = OTHER; note = N1-methylpseudouracil
  modified_base 918: mod_base = OTHER; note = N1-methylpseudouracil
  modified_base 920: mod_base = OTHER; note = N1-methylpseudouracil
  modified_base 922: mod_base = OTHER; note = N1-methylpseudouracil
  modified_base 923: mod_base = OTHER; note = N1-methylpseudouracil
  modified_base 933: mod_base = OTHER; note = N1-methylpseudouracil
  modified_base 937: mod_base = OTHER; note = N1-methylpseudouracil
  modified_base 941: mod_base = OTHER; note = N1-methylpseudouracil
  modified_base 942: mod_base = OTHER; note = N1-methylpseudouracil
  modified_base 950: mod_base = OTHER; note = N1-methylpseudouracil
  modified_base 965: mod_base = OTHER; note = N1-methylpseudouracil
  modified_base 969: mod_base = OTHER; note = N1-methylpseudouracil
  modified_base 975: mod_base = OTHER; note = N1-methylpseudouracil
  modified_base 977: mod_base = OTHER; note = N1-methylpseudouracil
  modified_base 985: mod_base = OTHER; note = N1-methylpseudouracil
  modified_base 986: mod_base = OTHER; note = N1-methylpseudouracil
  modified_base 997: mod_base = OTHER; note = N1-methylpseudouracil
  modified_base 998: mod_base = OTHER; note = N1-methylpseudouracil
  modified_base 1001: mod_base = OTHER; note = N1-methylpseudouracil
  modified_base 1016: mod_base = OTHER; note = N1-methylpseudouracil
  modified_base 1018: mod_base = OTHER; note = N1-methylpseudouracil
  modified_base 1027: mod_base = OTHER; note = N1-methylpseudouracil
  modified_base 1046: mod_base = OTHER; note = N1-methylpseudouracil
  modified_base 1047: mod_base = OTHER; note = N1-methylpseudouracil
  modified_base 1048: mod_base = OTHER; note = N1-methylpseudouracil
  modified_base 1049: mod_base = OTHER; note = N1-methylpseudouracil
  modified_base 1052: mod_base = OTHER; note = N1-methylpseudouracil
  modified_base 1058: mod_base = OTHER; note = N1-methylpseudouracil
  modified_base 1060: mod_base = OTHER; note = N1-methylpseudouracil
  modified_base 1061: mod_base = OTHER; note = N1-methylpseudouracil
  modified_base 1064: mod_base = OTHER; note = N1-methylpseudouracil
  modified_base 1066: mod_base = OTHER; note = N1-methylpseudouracil
  modified_base 1072: mod_base = OTHER; note = N1-methylpseudouracil
  modified_base 1086: mod_base = OTHER; note = N1-methylpseudouracil
  modified_base 1093: mod_base = OTHER; note = N1-methylpseudouracil
  modified_base 1100: mod_base = OTHER; note = N1-methylpseudouracil
  modified_base 1103: mod_base = OTHER; note = N1-methylpseudouracil
  modified_base 1106: mod_base = OTHER; note = N1-methylpseudouracil
  modified_base 1109: mod_base = OTHER; note = N1-methylpseudouracil
  modified_base 1112: mod_base = OTHER; note = N1-methylpseudouracil
  modified_base 1118: mod_base = OTHER; note = N1-methylpseudouracil
  modified_base 1122: mod_base = OTHER; note = N1-methylpseudouracil
  modified_base 1129: mod_base = OTHER; note = N1-methylpseudouracil
  modified_base 1137: mod_base = OTHER; note = N1-methylpseudouracil
  modified_base 1142: mod_base = OTHER; note = N1-methylpseudouracil
  modified_base 1150: mod_base = OTHER; note = N1-methylpseudouracil
  modified_base 1153: mod_base = OTHER; note = N1-methylpseudouracil
  modified_base 1170: mod_base = OTHER; note = N1-methylpseudouracil
  modified_base 1177: mod_base = OTHER; note = N1-methylpseudouracil
  modified_base 1179: mod_base = OTHER; note = N1-methylpseudouracil
  modified_base 1180: mod_base = OTHER; note = N1-methylpseudouracil
  modified_base 1190: mod_base = OTHER; note = N1-methylpseudouracil
  modified_base 1192: mod_base = OTHER; note = N1-methylpseudouracil
  modified_base 1193: mod_base = OTHER; note = N1-methylpseudouracil
  modified_base 1201: mod_base = OTHER; note = N1-methylpseudouracil
  modified_base 1202: mod_base = OTHER; note = N1-methylpseudouracil
  modified_base 1209: mod_base = OTHER; note = N1-methylpseudouracil
  modified_base 1211: mod_base = OTHER; note = N1-methylpseudouracil
  modified_base 1214: mod_base = OTHER; note = N1-methylpseudouracil
  modified_base 1224: mod_base = OTHER; note = N1-methylpseudouracil
  modified_base 1232: mod_base = OTHER; note = N1-methylpseudouracil
  modified_base 1235: mod_base = OTHER; note = N1-methylpseudouracil
  modified_base 1246: mod_base = OTHER; note = N1-methylpseudouracil
  modified_base 1248: mod_base = OTHER; note = N1-methylpseudouracil
  modified_base 1253: mod_base = OTHER; note = N1-methylpseudouracil
  modified_base 1255: mod_base = OTHER; note = N1-methylpseudouracil
  modified_base 1256: mod_base = OTHER; note = N1-methylpseudouracil
  modified_base 1265: mod_base = OTHER; note = N1-methylpseudouracil
  modified_base 1273: mod_base = OTHER; note = N1-methylpseudouracil
  modified_base 1279: mod_base = OTHER; note = N1-methylpseudouracil
  modified_base 1280: mod_base = OTHER; note = N1-methylpseudouracil
  modified_base 1292: mod_base = OTHER; note = N1-methylpseudouracil
  modified_base 1294: mod_base = OTHER; note = N1-methylpseudouracil
  modified_base 1295: mod_base = OTHER; note = N1-methylpseudouracil
  modified_base 1298: mod_base = OTHER; note = N1-methylpseudouracil
  modified_base 1303: mod_base = OTHER; note = N1-methylpseudouracil
  modified_base 1316: mod_base = OTHER; note = N1-methylpseudouracil
  modified_base 1320: mod_base = OTHER; note = N1-methylpseudouracil
  modified_base 1325: mod_base = OTHER; note = N1-methylpseudouracil
  modified_base 1329: mod_base = OTHER; note = N1-methylpseudouracil
  modified_base 1335: mod_base = OTHER; note = N1-methylpseudouracil
  modified_base 1340: mod_base = OTHER; note = N1-methylpseudouracil
  modified_base 1343: mod_base = OTHER; note = N1-methylpseudouracil
  modified_base 1349: mod_base = OTHER; note = N1-methylpseudouracil
  modified_base 1350: mod_base = OTHER; note = N1-methylpseudouracil
  modified_base 1353: mod_base = OTHER; note = N1-methylpseudouracil
  modified_base 1361: mod_base = OTHER; note = N1-methylpseudouracil
  modified_base 1370: mod_base = OTHER; note = N1-methylpseudouracil
  modified_base 1381: mod_base = OTHER; note = N1-methylpseudouracil
  modified_base 1384: mod_base = OTHER; note = N1-methylpseudouracil
  modified_base 1386: mod_base = OTHER; note = N1-methylpseudouracil
  modified_base 1401: mod_base = OTHER; note = N1-methylpseudouracil
  modified_base 1409: mod_base = OTHER; note = N1-methylpseudouracil
  modified_base 1413: mod_base = OTHER; note = N1-methylpseudouracil
  modified_base 1414: mod_base = OTHER; note = N1-methylpseudouracil
  modified_base 1426: mod_base = OTHER; note = N1-methylpseudouracil
  modified_base 1430: mod_base = OTHER; note = N1-methylpseudouracil
  modified_base 1432: mod_base = OTHER; note = N1-methylpseudouracil
  modified_base 1436: mod_base = OTHER; note = N1-methylpseudouracil
  modified_base 1437: mod_base = OTHER; note = N1-methylpseudouracil
  modified_base 1439: mod_base = OTHER; note = N1-methylpseudouracil
  modified_base 1442: mod_base = OTHER; note = N1-methylpseudouracil
  modified_base 1451: mod_base = OTHER; note = N1-methylpseudouracil
  modified_base 1453: mod_base = OTHER; note = N1-methylpseudouracil
  modified_base 1457: mod_base = OTHER; note = N1-methylpseudouracil
  modified_base 1460: mod_base = OTHER; note = N1-methylpseudouracil
  modified_base 1476: mod_base = OTHER; note = N1-methylpseudouracil
  modified_base 1478: mod_base = OTHER; note = N1-methylpseudouracil
  modified_base 1490: mod_base = OTHER; note = N1-methylpseudouracil
  modified_base 1498: mod_base = OTHER; note = N1-methylpseudouracil
  modified_base 1500: mod_base = OTHER; note = N1-methylpseudouracil
  modified_base 1501: mod_base = OTHER; note = N1-methylpseudouracil
  modified_base 1513: mod_base = OTHER; note = N1-methylpseudouracil
  modified_base 1514: mod_base = OTHER; note = N1-methylpseudouracil
  modified_base 1517: mod_base = OTHER; note = N1-methylpseudouracil
  modified_base 1521: mod_base = OTHER; note = N1-methylpseudouracil
  modified_base 1530: mod_base = OTHER; note = N1-methylpseudouracil
  modified_base 1531: mod_base = OTHER; note = N1-methylpseudouracil
  modified_base 1534: mod_base = OTHER; note = N1-methylpseudouracil
  modified_base 1535: mod_base = OTHER; note = N1-methylpseudouracil
  modified_base 1544: mod_base = OTHER; note = N1-methylpseudouracil
  modified_base 1550: mod_base = OTHER; note = N1-methylpseudouracil
  modified_base 1551: mod_base = OTHER; note = N1-methylpseudouracil
  modified_base 1560: mod_base = OTHER; note = N1-methylpseudouracil
  modified_base 1561: mod_base = OTHER; note = N1-methylpseudouracil
  modified_base 1565: mod_base = OTHER; note = N1-methylpseudouracil
  modified_base 1569: mod_base = OTHER; note = N1-methylpseudouracil
  modified_base 1576: mod_base = OTHER; note = N1-methylpseudouracil
  modified_base 1577: mod_base = OTHER; note = N1-methylpseudouracil
  modified_base 1590: mod_base = OTHER; note = N1-methylpseudouracil
  modified_base 1591: mod_base = OTHER; note = N1-methylpseudouracil
  modified_base 1592: mod_base = OTHER; note = N1-methylpseudouracil
  modified_base 1597: mod_base = OTHER; note = N1-methylpseudouracil
  modified_base 1598: mod_base = OTHER; note = N1-methylpseudouracil
  modified_base 1610: mod_base = OTHER; note = N1-methylpseudouracil
  modified_base 1612: mod_base = OTHER; note = N1-methylpseudouracil
  modified_base 1614: mod_base = OTHER; note = N1-methylpseudouracil
  modified_base 1617: mod_base = OTHER; note = N1-methylpseudouracil
  modified_base 1619: mod_base = OTHER; note = N1-methylpseudouracil
  modified_base 1640: mod_base = OTHER; note = N1-methylpseudouracil
  modified_base 1658: mod_base = OTHER; note = N1-methylpseudouracil
  modified_base 1661: mod_base = OTHER; note = N1-methylpseudouracil
  modified_base 1666: mod_base = OTHER; note = N1-methylpseudouracil
  modified_base 1667: mod_base = OTHER; note = N1-methylpseudouracil
  modified_base 1670: mod_base = OTHER; note = N1-methylpseudouracil
  modified_base 1703: mod_base = OTHER; note = N1-methylpseudouracil
  modified_base 1704: mod_base = OTHER; note = N1-methylpseudouracil
  modified_base 1712: mod_base = OTHER; note = N1-methylpseudouracil
  modified_base 1715: mod_base = OTHER; note = N1-methylpseudouracil
  modified_base 1723: mod_base = OTHER; note = N1-methylpseudouracil
  modified_base 1724: mod_base = OTHER; note = N1-methylpseudouracil
  modified_base 1732: mod_base = OTHER; note = N1-methylpseudouracil
  modified_base 1733: mod_base = OTHER; note = N1-methylpseudouracil
  modified_base 1736: mod_base = OTHER; note = N1-methylpseudouracil
  modified_base 1744: mod_base = OTHER; note = N1-methylpseudouracil
  modified_base 1746: mod_base = OTHER; note = N1-methylpseudouracil
  modified_base 1747: mod_base = OTHER; note = N1-methylpseudouracil
  modified_base 1749: mod_base = OTHER; note = N1-methylpseudouracil
  modified_base 1770: mod_base = OTHER; note = N1-methylpseudouracil
  modified_base 1771: mod_base = OTHER; note = N1-methylpseudouracil
  modified_base 1774: mod_base = OTHER; note = N1-methylpseudouracil
  modified_base 1775: mod_base = OTHER; note = N1-methylpseudouracil
  modified_base 1795: mod_base = OTHER; note = N1-methylpseudouracil
  modified_base 1796: mod_base = OTHER; note = N1-methylpseudouracil
  modified_base 1799: mod_base = OTHER; note = N1-methylpseudouracil
  modified_base 1807: mod_base = OTHER; note = N1-methylpseudouracil
  modified_base 1809: mod_base = OTHER; note = N1-methylpseudouracil
  modified_base 1815: mod_base = OTHER; note = N1-methylpseudouracil
  modified_base 1819: mod_base = OTHER; note = N1-methylpseudouracil
  modified_base 1820: mod_base = OTHER; note = N1-methylpseudouracil
  modified_base 1826: mod_base = OTHER; note = N1-methylpseudouracil
  modified_base 1830: mod_base = OTHER; note = N1-methylpseudouracil
  modified_base 1845: mod_base = OTHER; note = N1-methylpseudouracil
  modified_base 1855: mod_base = OTHER; note = N1-methylpseudouracil
  modified_base 1874: mod_base = OTHER; note = N1-methylpseudouracil
  modified_base 1880: mod_base = OTHER; note = N1-methylpseudouracil
  modified_base 1889: mod_base = OTHER; note = N1-methylpseudouracil
  modified_base 1892: mod_base = OTHER; note = N1-methylpseudouracil
  modified_base 1895: mod_base = OTHER; note = N1-methylpseudouracil
  modified_base 1900: mod_base = OTHER; note = N1-methylpseudouracil
  modified_base 1901: mod_base = OTHER; note = N1-methylpseudouracil
  modified_base 1907: mod_base = OTHER; note = N1-methylpseudouracil
  modified_base 1910: mod_base = OTHER; note = N1-methylpseudouracil
  modified_base 1913: mod_base = OTHER; note = N1-methylpseudouracil
  modified_base 1922: mod_base = OTHER; note = N1-methylpseudouracil
  modified_base 1930: mod_base = OTHER; note = N1-methylpseudouracil
  modified_base 1946: mod_base = OTHER; note = N1-methylpseudouracil
  modified_base 1955: mod_base = OTHER; note = N1-methylpseudouracil
  modified_base 1958: mod_base = OTHER; note = N1-methylpseudouracil
  modified_base 1963: mod_base = OTHER; note = N1-methylpseudouracil
  modified_base 1964: mod_base = OTHER; note = N1-methylpseudouracil
  modified_base 1969: mod_base = OTHER; note = N1-methylpseudouracil
  modified_base 1970: mod_base = OTHER; note = N1-methylpseudouracil
  modified_base 1972: mod_base = OTHER; note = N1-methylpseudouracil
  modified_base 1976: mod_base = OTHER; note = N1-methylpseudouracil
  modified_base 1980: mod_base = OTHER; note = N1-methylpseudouracil
  modified_base 1992: mod_base = OTHER; note = N1-methylpseudouracil
  modified_base 1997: mod_base = OTHER; note = N1-methylpseudouracil
  modified_base 2006: mod_base = OTHER; note = N1-methylpseudouracil
  modified_base 2007: mod_base = OTHER; note = N1-methylpseudouracil
  modified_base 2011: mod_base = OTHER; note = N1-methylpseudouracil
  modified_base 2015: mod_base = OTHER; note = N1-methylpseudouracil
  modified_base 2027: mod_base = OTHER; note = N1-methylpseudouracil
  modified_base 2028: mod_base = OTHER; note = N1-methylpseudouracil
  modified_base 2036: mod_base = OTHER; note = N1-methylpseudouracil
  modified_base 2048: mod_base = OTHER; note = N1-methylpseudouracil
  modified_base 2054: mod_base = OTHER; note = N1-methylpseudouracil
  modified_base 2061: mod_base = OTHER; note = N1-methylpseudouracil
  modified_base 2064: mod_base = OTHER; note = N1-methylpseudouracil
  modified_base 2070: mod_base = OTHER; note = N1-methylpseudouracil
  modified_base 2071: mod_base = OTHER; note = N1-methylpseudouracil
  modified_base 2072: mod_base = OTHER; note = N1-methylpseudouracil
  modified_base 2078: mod_base = OTHER; note = N1-methylpseudouracil
  modified_base 2085: mod_base = OTHER; note = N1-methylpseudouracil
  modified_base 2093: mod_base = OTHER; note = N1-methylpseudouracil
  modified_base 2094: mod_base = OTHER; note = N1-methylpseudouracil
  modified_base 2096: mod_base = OTHER; note = N1-methylpseudouracil
  modified_base 2097: mod_base = OTHER; note = N1-methylpseudouracil
  modified_base 2105: mod_base = OTHER; note = N1-methylpseudouracil
  modified_base 2111: mod_base = OTHER; note = N1-methylpseudouracil
  modified_base 2114: mod_base = OTHER; note = N1-methylpseudouracil
  modified_base 2117: mod_base = OTHER; note = N1-methylpseudouracil
  modified_base 2120: mod_base = OTHER; note = N1-methylpseudouracil
  modified_base 2125: mod_base = OTHER; note = N1-methylpseudouracil
  modified_base 2126: mod_base = OTHER; note = N1-methylpseudouracil
  modified_base 2134: mod_base = OTHER; note = N1-methylpseudouracil
  modified_base 2146: mod_base = OTHER; note = N1-methylpseudouracil
  modified_base 2147: mod_base = OTHER; note = N1-methylpseudouracil
  modified_base 2150: mod_base = OTHER; note = N1-methylpseudouracil
  modified_base 2152: mod_base = OTHER; note = N1-methylpseudouracil
  modified_base 2158: mod_base = OTHER; note = N1-methylpseudouracil
  modified_base 2180: mod_base = OTHER; note = N1-methylpseudouracil
  modified_base 2183: mod_base = OTHER; note = N1-methylpseudouracil
  modified_base 2188: mod_base = OTHER; note = N1-methylpseudouracil
  modified_base 2189: mod_base = OTHER; note = N1-methylpseudouracil
  modified_base 2192: mod_base = OTHER; note = N1-methylpseudouracil
  modified_base 2200: mod_base = OTHER; note = N1-methylpseudouracil
  modified_base 2204: mod_base = OTHER; note = N1-methylpseudouracil
  modified_base 2209: mod_base = OTHER; note = N1-methylpseudouracil
  modified_base 2210: mod_base = OTHER; note = N1-methylpseudouracil
  modified_base 2212: mod_base = OTHER; note = N1-methylpseudouracil
  modified_base 2222: mod_base = OTHER; note = N1-methylpseudouracil
  modified_base 2225: mod_base = OTHER; note = N1-methylpseudouracil
  modified_base 2227: mod_base = OTHER; note = N1-methylpseudouracil
  modified_base 2229: mod_base = OTHER; note = N1-methylpseudouracil
  modified_base 2232: mod_base = OTHER; note = N1-methylpseudouracil
  modified_base 2234: mod_base = OTHER; note = N1-methylpseudouracil
angaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacancngnacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngacc

SEQ ID NO: 26 - RNA, 57 nt, synthetic construct
  source 1..57: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
gggaaanaagagagaaaagaagagnaagaagaaananaagaccccggcgccgccacc

SEQ ID NO: 27 - RNA, 119 nt, synthetic construct
  source 1..119: mol_type = other RNA
  modified_base 1: mod_base = OTHER; note = N1-methylpseudouracil
  modified_base 4: mod_base = OTHER; note = N1-methylpseudouracil
  modified_base 7: mod_base = OTHER; note = N1-methylpseudouracil
  modified_base 12: mod_base = OTHER; note = N1-methylpseudouracil
  modified_base 19: mod_base = OTHER; note = N1-methylpseudouracil
  modified_base 23: mod_base = OTHER; note = N1-methylpseudouracil
  modified_base 28: mod_base = OTHER; note = N1-methylpseudouracil
  modified_base 32: mod_base = OTHER; note = N1-methylpseudouracil
  modified_base 33: mod_base = OTHER; note = N1-methylpseudouracil
  modified_base 35: mod_base = OTHER; note = N1-methylpseudouracil
  modified_base 36: mod_base = OTHER; note = N1-methylpseudouracil
  modified_base 42: mod_base = OTHER; note = N1-methylpseudouracil
  modified_base 43: mod_base = OTHER; note = N1-methylpseudouracil
  modified_base 49: mod_base = OTHER; note = N1-methylpseudouracil
  modified_base 62: mod_base = OTHER; note = N1-methylpseudouracil
  modified_base 65: mod_base = OTHER; note = N1-methylpseudouracil
  modified_base 70: mod_base = OTHER; note = N1-methylpseudouracil
  modified_base 71: mod_base = OTHER; note = N1-methylpseudouracil
  modified_base 74: mod_base = OTHER; note = N1-methylpseudouracil
  modified_base 82: mod_base = OTHER; note = N1-methylpseudouracil
  modified_base 90: mod_base = OTHER; note = N1-methylpseudouracil
  modified_base 93: mod_base = OTHER; note = N1-methylpseudouracil
  modified_base 95: mod_base = OTHER; note = N1-methylpseudouracil
  modified_base 96: mod_base = OTHER; note = N1-methylpseudouracil
  modified_base 97: mod_base = OTHER; note = N1-methylpseudouracil
  modified_base 101: mod_base = OTHER; note = N1-methylpseudouracil
  modified_base 106: mod_base = OTHER; note = N1-methylpseudouracil
  modified_base 108: mod_base = OTHER; note = N1-methylpseudouracil
  modified_base 112: mod_base = OTHER; note = N1-methylpseudouracil
nganaanaggcnggagccncggnggccnagcnncnngccccnngggccnccccccagccccnccnccccnnccngcacccgnacccccgnggncnnngaanaaagncngagngggcggc

SEQ ID NO: 28 - RNA, 2514 nt, synthetic construct
  source 1..2514: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 59: mod_base = OTHER; note = N1-methylpseudouracil
  modified_base 64: mod_base = OTHER; note = N1-methylpseudouracil
  modified_base 68: mod_base = OTHER; note = N1-methylpseudouracil
  modified_base 73: mod_base = OTHER; note = N1-methylpseudouracil
  modified_base 74: mod_base = OTHER; note = N1-methylpseudouracil
  modified_base 77: mod_base = OTHER; note = N1-methylpseudouracil
  modified_base 83: mod_base = OTHER; note = N1-methylpseudouracil
  modified_base 86: mod_base = OTHER; note = N1-methylpseudouracil
  modified_base 88: mod_base = OTHER; note = N1-methylpseudouracil
  modified_base 89: mod_base = OTHER; note = N1-methylpseudouracil
  modified_base 92: mod_base = OTHER; note = N1-methylpseudouracil
  modified_base 94: mod_base = OTHER; note = N1-methylpseudouracil
  modified_base 95: mod_base = OTHER; note = N1-methylpseudouracil
  modified_base 106: mod_base = OTHER; note = N1-methylpseudouracil
  modified_base 119: mod_base = OTHER; note = N1-methylpseudouracil
  modified_base 121: mod_base = OTHER; note = N1-methylpseudouracil
  modified_base 122: mod_base = OTHER; note = N1-methylpseudouracil
  modified_base 149: mod_base = OTHER; note = N1-methylpseudouracil
  modified_base 160: mod_base = OTHER; note = N1-methylpseudouracil
  modified_base 161: mod_base = OTHER; note = N1-methylpseudouracil
  modified_base 170: mod_base = OTHER; note = N1-methylpseudouracil
  modified_base 184: mod_base = OTHER; note = N1-methylpseudouracil
  modified_base 185: mod_base = OTHER; note = N1-methylpseudouracil
  modified_base 194: mod_base = OTHER; note = N1-methylpseudouracil
  modified_base 197: mod_base = OTHER; note = N1-methylpseudouracil
  modified_base 200: mod_base = OTHER; note = N1-methylpseudouracil
  modified_base 203: mod_base = OTHER; note = N1-methylpseudouracil
  modified_base 208: mod_base = OTHER; note = N1-methylpseudouracil
  modified_base 209: mod_base = OTHER; note = N1-methylpseudouracil
  modified_base 217: mod_base = OTHER; note = N1-methylpseudouracil
  modified_base 221: mod_base = OTHER; note = N1-methylpseudouracil
  modified_base 229: mod_base = OTHER; note = N1-methylpseudouracil
  modified_base 234: mod_base = OTHER; note = N1-methylpseudouracil
  modified_base 235: mod_base = OTHER; note = N1-methylpseudouracil
  modified_base 236: mod_base = OTHER; note = N1-methylpseudouracil
  modified_base 248: mod_base = OTHER; note = N1-methylpseudouracil
  modified_base 254: mod_base = OTHER; note = N1-methylpseudouracil
  modified_base 257: mod_base = OTHER; note = N1-methylpseudouracil
  modified_base 266: mod_base = OTHER; note = N1-methylpseudouracil
  modified_base 274: mod_base = OTHER; note = N1-methylpseudouracil
  modified_base 275: mod_base = OTHER; note = N1-methylpseudouracil
  modified_base 286: mod_base = OTHER; note = N1-methylpseudouracil
  modified_base 290: mod_base = OTHER; note = N1-methylpseudouracil
  modified_base 313: mod_base = OTHER; note = N1-methylpseudouracil
  modified_base 326: mod_base = OTHER; note = N1-methylpseudouracil
  modified_base 335: mod_base = OTHER; note = N1-methylpseudouracil
  modified_base 337: mod_base = OTHER; note = N1-methylpseudouracil
  modified_base 338: mod_base = OTHER; note = N1-methylpseudouracil
  modified_base 350: mod_base = OTHER; note = N1-methylpseudouracil
  modified_base 352: mod_base = OTHER; note = N1-methylpseudouracil
  modified_base 359: mod_base = OTHER; note = N1-methylpseudouracil
  modified_base 368: mod_base = OTHER; note = N1-methylpseudouracil
  modified_base 378: mod_base = OTHER; note = N1-methylpseudouracil
  modified_base 379: mod_base = OTHER; note = N1-methylpseudouracil
  modified_base 389: mod_base = OTHER; note = N1-methylpseudouracil
  modified_base 397: mod_base = OTHER; note = N1-methylpseudouracil
  modified_base 400: mod_base = OTHER; note = N1-methylpseudouracil
  modified_base 430: mod_base = OTHER; note = N1-methylpseudouracil
  modified_base 433: mod_base = OTHER; note = N1-methylpseudouracil
  modified_base 434: mod_base = OTHER; note = N1-methylpseudouracil
  modified_base 437: mod_base = OTHER; note = N1-methylpseudouracil
  modified_base 459: mod_base = OTHER; note = N1-methylpseudouracil
  modified_base 464: mod_base = OTHER; note = N1-methylpseudouracil
  modified_base 468: mod_base = OTHER; note = N1-methylpseudouracil
  modified_base 473: mod_base = OTHER; note = N1-methylpseudouracil
  modified_base 476: mod_base = OTHER; note = N1-methylpseudouracil
  modified_base 483: mod_base = OTHER; note = N1-methylpseudouracil
  modified_base 488: mod_base = OTHER; note = N1-methylpseudouracil
  modified_base 494: mod_base = OTHER; note = N1-methylpseudouracil
  modified_base 497: mod_base = OTHER; note = N1-methylpseudouracil
  modified_base 499: mod_base = OTHER; note = N1-methylpseudouracil
  modified_base 501: mod_base = OTHER; note = N1-methylpseudouracil
  modified_base 508: mod_base = OTHER; note = N1-methylpseudouracil
  modified_base 509: mod_base = OTHER; note = N1-methylpseudouracil
  modified_base 529: mod_base = OTHER; note = N1-methylpseudouracil
  modified_base 530: mod_base = OTHER; note = N1-methylpseudouracil
  modified_base 533: mod_base = OTHER; note = N1-methylpseudouracil
  modified_base 541: mod_base = OTHER; note = N1-methylpseudouracil
  modified_base 545: mod_base = OTHER; note = N1-methylpseudouracil
  modified_base 547: mod_base = OTHER; note = N1-methylpseudouracil
  modified_base 554: mod_base = OTHER; note = N1-methylpseudouracil
  modified_base 570: mod_base = OTHER; note = N1-methylpseudouracil
  modified_base 571: mod_base = OTHER; note = N1-methylpseudouracil
  modified_base 574: mod_base = OTHER; note = N1-methylpseudouracil
  modified_base 575: mod_base = OTHER; note = N1-methylpseudouracil
  modified_base 577: mod_base = OTHER; note = N1-methylpseudouracil
  modified_base 585: mod_base = OTHER; note = N1-methylpseudouracil
  modified_base 589: mod_base = OTHER; note = N1-methylpseudouracil
  modified_base 590: mod_base = OTHER; note = N1-methylpseudouracil
  modified_base 593: mod_base = OTHER; note = N1-methylpseudouracil
  modified_base 595: mod_base = OTHER; note = N1-methylpseudouracil
  modified_base 596: mod_base = OTHER; note = N1-methylpseudouracil
  modified_base 598: mod_base = OTHER; note = N1-methylpseudouracil
  modified_base 599: mod_base = OTHER; note = N1-methylpseudouracil
  modified_base 610: mod_base = OTHER; note = N1-methylpseudouracil
  modified_base 622: mod_base = OTHER; note = N1-methylpseudouracil
  modified_base 623: mod_base = OTHER; note = N1-methylpseudouracil
  modified_base 631: mod_base = OTHER; note = N1-methylpseudouracil
  modified_base 634: mod_base = OTHER; note = N1-methylpseudouracil
  modified_base 648: mod_base = OTHER; note = N1-methylpseudouracil
  modified_base 658: mod_base = OTHER; note = N1-methylpseudouracil
  modified_base 662: mod_base = OTHER; note = N1-methylpseudouracil
  modified_base 665: mod_base = OTHER; note = N1-methylpseudouracil
  modified_base 669: mod_base = OTHER; note = N1-methylpseudouracil
  modified_base 674: mod_base = OTHER; note = N1-methylpseudouracil
  modified_base 683: mod_base = OTHER; note = N1-methylpseudouracil
  modified_base 703: mod_base = OTHER; note = N1-methylpseudouracil
  modified_base 711: mod_base = OTHER; note = N1-methylpseudouracil
  modified_base 722: mod_base = OTHER; note = N1-methylpseudouracil
  modified_base 727: mod_base = OTHER; note = N1-methylpseudouracil
  modified_base 737: mod_base = OTHER; note = N1-methylpseudouracil
  modified_base 745: mod_base = OTHER; note = N1-methylpseudouracil
  modified_base 746: mod_base = OTHER; note = N1-methylpseudouracil
  modified_base 759: mod_base = OTHER; note = N1-methylpseudouracil
  modified_base 760: mod_base = OTHER; note = N1-methylpseudouracil
  modified_base 761: mod_base = OTHER; note = N1-methylpseudouracil
  modified_base 769: mod_base = OTHER; note = N1-methylpseudouracil
  modified_base 776: mod_base = OTHER; note = N1-methylpseudouracil
  modified_base 780: mod_base = OTHER; note = N1-methylpseudouracil
  modified_base 784: mod_base = OTHER; note = N1-methylpseudouracil
  modified_base 785: mod_base = OTHER; note = N1-methylpseudouracil
  modified_base 796: mod_base = OTHER; note = N1-methylpseudouracil
  modified_base 797: mod_base = OTHER; note = N1-methylpseudouracil
  modified_base 801: mod_base = OTHER; note = N1-methylpseudouracil
  modified_base 811: mod_base = OTHER; note = N1-methylpseudouracil
  modified_base 812: mod_base = OTHER; note = N1-methylpseudouracil
  modified_base 816: mod_base = OTHER; note = N1-methylpseudouracil
  modified_base 821: mod_base = OTHER; note = N1-methylpseudouracil
  modified_base 830: mod_base = OTHER; note = N1-methylpseudouracil
  modified_base 833: mod_base = OTHER; note = N1-methylpseudouracil
  modified_base 837: mod_base = OTHER; note = N1-methylpseudouracil
  modified_base 842: mod_base = OTHER; note = N1-methylpseudouracil
  modified_base 851: mod_base = OTHER; note = N1-methylpseudouracil
  modified_base 855: mod_base = OTHER; note = N1-methylpseudouracil
  modified_base 862: mod_base = OTHER; note = N1-methylpseudouracil
  modified_base 865: mod_base = OTHER; note = N1-methylpseudouracil
  modified_base 867: mod_base = OTHER; note = N1-methylpseudouracil
  modified_base 878: mod_base = OTHER; note = N1-methylpseudouracil
  modified_base 881: mod_base = OTHER; note = N1-methylpseudouracil
  modified_base 886: mod_base = OTHER; note = N1-methylpseudouracil
  modified_base 908: mod_base = OTHER; note = N1-methylpseudouracil
  modified_base 916: mod_base = OTHER; note = N1-methylpseudouracil
  modified_base 920: mod_base = OTHER; note = N1-methylpseudouracil
  modified_base 922: mod_base = OTHER; note = N1-methylpseudouracil
  modified_base 941: mod_base = OTHER; note = N1-methylpseudouracil
  modified_base 953: mod_base = OTHER; note = N1-methylpseudouracil
  modified_base 961: mod_base = OTHER; note = N1-methylpseudouracil
  modified_base 963: mod_base = OTHER; note = N1-methylpseudouracil
  modified_base 964: mod_base = OTHER; note = N1-methylpseudouracil
  modified_base 966: mod_base = OTHER; note = N1-methylpseudouracil
  modified_base 975: mod_base = OTHER; note = N1-methylpseudouracil
  modified_base 977: mod_base = OTHER; note = N1-methylpseudouracil
  modified_base 979: mod_base = OTHER; note = N1-methylpseudouracil
  modified_base 980: mod_base = OTHER; note = N1-methylpseudouracil
  modified_base 990: mod_base = OTHER; note = N1-methylpseudouracil
  modified_base 994: mod_base = OTHER; note = N1-methylpseudouracil
  modified_base 998: mod_base = OTHER; note = N1-methylpseudouracil
  modified_base 999: mod_base = OTHER; note = N1-methylpseudouracil
  modified_base 1007: mod_base = OTHER; note = N1-methylpseudouracil
  modified_base 1022: mod_base = OTHER; note = N1-methylpseudouracil
  modified_base 1026: mod_base = OTHER; note = N1-methylpseudouracil
  modified_base 1032: mod_base = OTHER; note = N1-methylpseudouracil
  modified_base 1034: mod_base = OTHER; note = N1-methylpseudouracil
  modified_base 1042: mod_base = OTHER; note = N1-methylpseudouracil
  modified_base 1043: mod_base = OTHER; note = N1-methylpseudouracil
  modified_base 1054: mod_base = OTHER; note = N1-methylpseudouracil
  modified_base 1055: mod_base = OTHER; note = N1-methylpseudouracil
  modified_base 1058: mod_base = OTHER; note = N1-methylpseudouracil
  modified_base 1073: mod_base = OTHER; note = N1-methylpseudouracil
  modified_base 1075: mod_base = OTHER; note = N1-methylpseudouracil
  modified_base 1084: mod_base = OTHER; note = N1-methylpseudouracil
  modified_base 1103: mod_base = OTHER; note = N1-methylpseudouracil
  modified_base 1104: mod_base = OTHER; note = N1-methylpseudouracil
  modified_base 1105: mod_base = OTHER; note = N1-methylpseudouracil
  modified_base 1106: mod_base = OTHER; note = N1-methylpseudouracil
  modified_base 1109: mod_base = OTHER; note = N1-methylpseudouracil
  modified_base 1115: mod_base = OTHER; note = N1-methylpseudouracil
  modified_base 1117: mod_base = OTHER; note = N1-methylpseudouracil
  modified_base 1118: mod_base = OTHER; note = N1-methylpseudouracil
  modified_base 1121: mod_base = OTHER; note = N1-methylpseudouracil
  modified_base 1123: mod_base = OTHER; note = N1-methylpseudouracil
  modified_base 1129: mod_base = OTHER; note = N1-methylpseudouracil
  modified_base 1143: mod_base = OTHER; note = N1-methylpseudouracil
  modified_base 1150: mod_base = OTHER; note = N1-methylpseudouracil
  modified_base 1157: mod_base = OTHER; note = N1-methylpseudouracil
  modified_base 1160: mod_base = OTHER; note = N1-methylpseudouracil
  modified_base 1163: mod_base = OTHER; note = N1-methylpseudouracil
  modified_base 1166: mod_base = OTHER; note = N1-methylpseudouracil
  modified_base 1169: mod_base = OTHER; note = N1-methylpseudouracil
  modified_base 1175: mod_base = OTHER; note = N1-methylpseudouracil
  modified_base 1179: mod_base = OTHER; note = N1-methylpseudouracil
  modified_base 1186: mod_base = OTHER; note = N1-methylpseudouracil
  modified_base 1194: mod_base = OTHER; note = N1-methylpseudouracil
  modified_base 1199: mod_base = OTHER; note = N1-methylpseudouracil
  modified_base 1207: mod_base = OTHER; note = N1-methylpseudouracil
  modified_base 1210: mod_base = OTHER; note = N1-methylpseudouracil
  modified_base 1227: mod_base = OTHER; note = N1-methylpseudouracil
  modified_base 1234: mod_base = OTHER; note = N1-methylpseudouracil
  modified_base 1236: mod_base = OTHER; note = N1-methylpseudouracil
  modified_base 1237: mod_base = OTHER; note = N1-methylpseudouracil
  modified_base 1247: mod_base = OTHER; note = N1-methylpseudouracil
  modified_base 1249: mod_base = OTHER; note = N1-methylpseudouracil
  modified_base 1250: mod_base = OTHER; note = N1-methylpseudouracil
  modified_base 1258: mod_base = OTHER; note = N1-methylpseudouracil
  modified_base 1259: mod_base = OTHER; note = N1-methylpseudouracil
  modified_base 1266: mod_base = OTHER; note = N1-methylpseudouracil
  modified_base 1268: mod_base = OTHER; note = N1-methylpseudouracil
  modified_base 1271: mod_base = OTHER; note = N1-methylpseudouracil
  modified_base 1281: mod_base = OTHER; note = N1-methylpseudouracil
  modified_base 1289: mod_base = OTHER; note = N1-methylpseudouracil
  modified_base 1292: mod_base = OTHER; note = N1-methylpseudouracil
  modified_base 1303: mod_base = OTHER; note = N1-methylpseudouracil
  modified_base 1305: mod_base = OTHER; note = N1-methylpseudouracil
  modified_base 1310: mod_base = OTHER; note = N1-methylpseudouracil
  modified_base 1312: mod_base = OTHER; note = N1-methylpseudouracil
  modified_base 1313: mod_base = OTHER; note = N1-methylpseudouracil
  modified_base 1322: mod_base = OTHER; note = N1-methylpseudouracil
  modified_base 1330: mod_base = OTHER; note = N1-methylpseudouracil
  modified_base 1336: mod_base = OTHER; note = N1-methylpseudouracil
  modified_base 1337: mod_base = OTHER; note = N1-methylpseudouracil
  modified_base 1349: mod_base = OTHER; note = N1-methylpseudouracil
  modified_base 1351: mod_base = OTHER; note = N1-methylpseudouracil
  modified_base 1352: mod_base = OTHER; note = N1-methylpseudouracil
  modified_base 1355: mod_base = OTHER; note = N1-methylpseudouracil
  modified_base 1360: mod_base = OTHER; note = N1-methylpseudouracil
  modified_base 1373: mod_base = OTHER; note = N1-methylpseudouracil
  modified_base 1377: mod_base = OTHER; note = N1-methylpseudouracil
  modified_base 1382: mod_base = OTHER; note = N1-methylpseudouracil
  modified_base 1386: mod_base = OTHER; note = N1-methylpseudouracil
  modified_base 1392: mod_base = OTHER; note = N1-methylpseudouracil
  modified_base 1397: mod_base = OTHER; note = N1-methylpseudouracil
  modified_base 1400: mod_base = OTHER; note = N1-methylpseudouracil
  modified_base 1406: mod_base = OTHER; note = N1-methylpseudouracil
  modified_base 1407: mod_base = OTHER; note = N1-methylpseudouracil
  modified_base 1410: mod_base = OTHER; note = N1-methylpseudouracil
  modified_base 1418: mod_base = OTHER; note = N1-methylpseudouracil
  modified_base 1427: mod_base = OTHER; note = N1-methylpseudouracil
  modified_base 1438: mod_base = OTHER; note = N1-methylpseudouracil
  modified_base 1441: mod_base = OTHER; note = N1-methylpseudouracil
  modified_base 1443: mod_base = OTHER; note = N1-methylpseudouracil
  modified_base 1458: mod_base = OTHER; note = N1-methylpseudouracil
  modified_base 1466: mod_base = OTHER; note = N1-methylpseudouracil
  modified_base 1470: mod_base = OTHER; note = N1-methylpseudouracil
  modified_base 1471: mod_base = OTHER; note = N1-methylpseudouracil
  modified_base 1483: mod_base = OTHER; note = N1-methylpseudouracil
  modified_base 1487: mod_base = OTHER; note = N1-methylpseudouracil
  modified_base 1489: mod_base = OTHER; note = N1-methylpseudouracil
  modified_base 1493: mod_base = OTHER; note = N1-methylpseudouracil
  modified_base 1494: mod_base = OTHER; note = N1-methylpseudouracil
  modified_base 1496: mod_base = OTHER; note = N1-methylpseudouracil
  modified_base 1499: mod_base = OTHER; note = N1-methylpseudouracil
  modified_base 1508: mod_base = OTHER; note = N1-methylpseudouracil
  modified_base 1510: mod_base = OTHER; note = N1-methylpseudouracil
  modified_base 1514: mod_base = OTHER; note = N1-methylpseudouracil
  modified_base 1517: mod_base = OTHER; note = N1-methylpseudouracil
  modified_base 1533: mod_base = OTHER; note = N1-methylpseudouracil
  modified_base 1535: mod_base = OTHER; note = N1-methylpseudouracil
  modified_base 1547: mod_base = OTHER; note = N1-methylpseudouracil
  modified_base 1555: mod_base = OTHER; note = N1-methylpseudouracil
  modified_base 1557: mod_base = OTHER; note = N1-methylpseudouracil
  modified_base 1558: mod_base = OTHER; note = N1-methylpseudouracil
  modified_base 1570: mod_base = OTHER; note = N1-methylpseudouracil
  modified_base 1571: mod_base = OTHER; note = N1-methylpseudouracil
  modified_base 1574: mod_base = OTHER; note = N1-methylpseudouracil
  modified_base 1578: mod_base = OTHER; note = N1-methylpseudouracil
  modified_base 1587: mod_base = OTHER; note = N1-methylpseudouracil
  modified_base 1588: mod_base = OTHER; note = N1-methylpseudouracil
  modified_base 1591: mod_base = OTHER; note = N1-methylpseudouracil
  modified_base 1592: mod_base = OTHER; note = N1-methylpseudouracil
  modified_base 1601: mod_base = OTHER; note = N1-methylpseudouracil
  modified_base 1607: mod_base = OTHER; note = N1-methylpseudouracil
  modified_base 1608: mod_base = OTHER; note = N1-methylpseudouracil
  modified_base 1617: mod_base = OTHER; note = N1-methylpseudouracil
  modified_base 1618: mod_base = OTHER; note = N1-methylpseudouracil
  modified_base 1622: mod_base = OTHER; note = N1-methylpseudouracil
  modified_base 1626: mod_base = OTHER; note = N1-methylpseudouracil
  modified_base 1633: mod_base = OTHER; note = N1-methylpseudouracil
  modified_base 1634: mod_base = OTHER; note = N1-methylpseudouracil
  modified_base 1647: mod_base = OTHER; note = N1-methylpseudouracil
  modified_base 1648: mod_base = OTHER; note = N1-methylpseudouracil
  modified_base 1649: mod_base = OTHER; note = N1-methylpseudouracil
  modified_base 1654: mod_base = OTHER; note = N1-methylpseudouracil
  modified_base 1655: mod_base = OTHER; note = N1-methylpseudouracil
  modified_base 1667: mod_base = OTHER; note = N1-methylpseudouracil
  modified_base 1669: mod_base = OTHER; note = N1-methylpseudouracil
  modified_base 1671: mod_base = OTHER; note = N1-methylpseudouracil
  modified_base 1674: mod_base = OTHER; note = N1-methylpseudouracil
  modified_base 1676: mod_base = OTHER; note = N1-methylpseudouracil
  modified_base 1697: mod_base = OTHER; note = N1-methylpseudouracil
  modified_base 1715: mod_base = OTHER; note = N1-methylpseudouracil
  modified_base 1718: mod_base = OTHER; note = N1-methylpseudouracil
  modified_base 1723: mod_base = OTHER; note = N1-methylpseudouracil
  modified_base 1724: mod_base = OTHER; note = N1-methylpseudouracil
  modified_base 1727: mod_base = OTHER; note = N1-methylpseudouracil
  modified_base 1760: mod_base = OTHER; note = N1-methylpseudouracil
  modified_base 1761: mod_base = OTHER; note = N1-methylpseudouracil
  modified_base 1769: mod_base = OTHER; note = N1-methylpseudouracil
  modified_base 1772: mod_base = OTHER; note = N1-methylpseudouracil
  modified_base 1780: mod_base = OTHER; note = N1-methylpseudouracil
  modified_base 1781: mod_base = OTHER; note = N1-methylpseudouracil
  modified_base 1789: mod_base = OTHER; note = N1-methylpseudouracil
  modified_base 1790: mod_base = OTHER; note = N1-methylpseudouracil
  modified_base 1793: mod_base = OTHER; note = N1-methylpseudouracil
  modified_base 1801: mod_base = OTHER; note = N1-methylpseudouracil
  modified_base 1803: mod_base = OTHER; note = N1-methylpseudouracil
  modified_base 1804: mod_base = OTHER; note = N1-methylpseudouracil
  modified_base 1806: mod_base = OTHER; note = N1-methylpseudouracil
  modified_base 1827: mod_base = OTHER; note = N1-methylpseudouracil
  modified_base 1828: mod_base = OTHER; note = N1-methylpseudouracil
  modified_base 1831: mod_base = OTHER; note = N1-methylpseudouracil
  modified_base 1832: mod_base = OTHER; note = N1-methylpseudouracil
  modified_base 1852: mod_base = OTHER; note = N1-methylpseudouracil
  modified_base 1853: mod_base = OTHER; note = N1-methylpseudouracil
  modified_base 1856: mod_base = OTHER; note = N1-methylpseudouracil
  modified_base 1864: mod_base = OTHER; note = N1-methylpseudouracil
  modified_base 1866: mod_base = OTHER; note = N1-methylpseudouracil
  modified_base 1872: mod_base = OTHER; note = N1-methylpseudouracil
  modified_base 1876: mod_base = OTHER; note = N1-methylpseudouracil
  modified_base 1877: mod_base = OTHER; note = N1-methylpseudouracil
  modified_base 1883: mod_base = OTHER; note = N1-methylpseudouracil
  modified_base 1887: mod_base = OTHER; note = N1-methylpseudouracil
  modified_base 1902: mod_base = OTHER; note = N1-methylpseudouracil
  modified_base 1912: mod_base = OTHER; note = N1-methylpseudouracil
  modified_base 1931: mod_base = OTHER; note = N1-methylpseudouracil
  modified_base 1937: mod_base = OTHER; note = N1-methylpseudouracil
  modified_base 1946: mod_base = OTHER; note = N1-methylpseudouracil
  modified_base 1949: mod_base = OTHER; note = N1-methylpseudouracil
  modified_base 1952: mod_base = OTHER; note = N1-methylpseudouracil
  modified_base 1957: mod_base = OTHER; note = N1-methylpseudouracil
  modified_base 1958: mod_base = OTHER; note = N1-methylpseudouracil
  modified_base 1964: mod_base = OTHER; note = N1-methylpseudouracil
  modified_base 1967: mod_base = OTHER; note = N1-methylpseudouracil
  modified_base 1970: mod_base = OTHER; note = N1-methylpseudouracil
  modified_base 1979: mod_base = OTHER; note = N1-methylpseudouracil
  modified_base 1987: mod_base = OTHER; note = N1-methylpseudouracil
  modified_base 2003: mod_base = OTHER; note = N1-methylpseudouracil
  modified_base 2012: mod_base = OTHER; note = N1-methylpseudouracil
  modified_base 2015: mod_base = OTHER; note = N1-methylpseudouracil
  modified_base 2020: mod_base = OTHER; note = N1-methylpseudouracil
  modified_base 2021: mod_base = OTHER; note = N1-methylpseudouracil
  modified_base 2026: mod_base = OTHER; note = N1-methylpseudouracil
  modified_base 2027: mod_base = OTHER; note = N1-methylpseudouracil
  modified_base 2029: mod_base = OTHER; note = N1-methylpseudouracil
  modified_base 2033: mod_base = OTHER; note = N1-methylpseudouracil
  modified_base 2037: mod_base = OTHER; note = N1-methylpseudouracil
  modified_base 2049: mod_base = OTHER; note = N1-methylpseudouracil
  modified_base 2054: mod_base = OTHER; note = N1-methylpseudouracil
  modified_base 2063: mod_base = OTHER; note = N1-methylpseudouracil
  modified_base 2064: mod_base = OTHER; note = N1-methylpseudouracil
  modified_base 2068: mod_base = OTHER; note = N1-methylpseudouracil
  modified_base 2072: mod_base = OTHER; note = N1-methylpseudouracil
  modified_base 2084: mod_base = OTHER; note = N1-methylpseudouracil
  modified_base 2085: mod_base = OTHER; note = N1-methylpseudouracil
  modified_base 2093: mod_base = OTHER; note = N1-methylpseudouracil
  modified_base 2105: mod_base = OTHER; note = N1-methylpseudouracil
  modified_base 2111: mod_base = OTHER; note = N1-methylpseudouracil
  modified_base 2118: mod_base = OTHER; note = N1-methylpseudouracil
  modified_base 2121: mod_base = OTHER; note = N1-methylpseudouracil
  modified_base 2127: mod_base = OTHER; note = N1-methylpseudouracil
  modified_base 2128: mod_base = OTHER; note = N1-methylpseudouracil
  modified_base 2129: mod_base = OTHER; note = N1-methylpseudouracil
  modified_base 2135: mod_base = OTHER; note = N1-methylpseudouracil
  modified_base 2142: mod_base = OTHER; note = N1-methylpseudouracil
  modified_base 2150: mod_base = OTHER; note = N1-methylpseudouracil
  modified_base 2151: mod_base = OTHER; note = N1-methylpseudouracil
  modified_base 2153: mod_base = OTHER; note = N1-methylpseudouracil
  modified_base 2154: mod_base = OTHER; note = N1-methylpseudouracil
  modified_base 2162: mod_base = OTHER; note = N1-methylpseudouracil
  modified_base 2168: mod_base = OTHER; note = N1-methylpseudouracil
  modified_base 2171: mod_base = OTHER; note = N1-methylpseudouracil
  modified_base 2174: mod_base = OTHER; note = N1-methylpseudouracil
  modified_base 2177: mod_base = OTHER; note = N1-methylpseudouracil
  modified_base 2182: mod_base = OTHER; note = N1-methylpseudouracil
  modified_base 2183: mod_base = OTHER; note = N1-methylpseudouracil
  modified_base 2191: mod_base = OTHER; note = N1-methylpseudouracil
  modified_base 2203: mod_base = OTHER; note = N1-methylpseudouracil
  modified_base 2204: mod_base = OTHER; note = N1-methylpseudouracil
  modified_base 2207: mod_base = OTHER; note = N1-methylpseudouracil
  modified_base 2209: mod_base = OTHER; note = N1-methylpseudouracil
  modified_base 2215: mod_base = OTHER; note = N1-methylpseudouracil
  modified_base 2237: mod_base = OTHER; note = N1-methylpseudouracil
  modified_base 2240: mod_base = OTHER; note = N1-methylpseudouracil
  modified_base 2245: mod_base = OTHER; note = N1-methylpseudouracil
  modified_base 2246: mod_base = OTHER; note = N1-methylpseudouracil
  modified_base 2249: mod_base = OTHER; note = N1-methylpseudouracil
  modified_base 2257: mod_base = OTHER; note = N1-methylpseudouracil
  modified_base 2261: mod_base = OTHER; note = N1-methylpseudouracil
  modified_base 2266: mod_base = OTHER; note = N1-methylpseudouracil
  modified_base 2267: mod_base = OTHER; note = N1-methylpseudouracil
  modified_base 2269: mod_base = OTHER; note = N1-methylpseudouracil
  modified_base 2279: mod_base = OTHER; note = N1-methylpseudouracil
  modified_base 2282: mod_base = OTHER; note = N1-methylpseudouracil
  modified_base 2284: mod_base = OTHER; note = N1-methylpseudouracil
  modified_base 2286: mod_base = OTHER; note = N1-methylpseudouracil
  modified_base 2289: mod_base = OTHER; note = N1-methylpseudouracil
  modified_base 2291: mod_base = OTHER; note = N1-methylpseudouracil
  modified_base 2296: mod_base = OTHER; note = N1-methylpseudouracil
  modified_base 2299: mod_base = OTHER; note = N1-methylpseudouracil
  modified_base 2302: mod_base = OTHER; note = N1-methylpseudouracil
  modified_base 2307: mod_base = OTHER; note = N1-methylpseudouracil
  modified_base 2314: mod_base = OTHER; note = N1-methylpseudouracil
  modified_base 2318: mod_base = OTHER; note = N1-methylpseudouracil
  modified_base 2323: mod_base = OTHER; note = N1-methylpseudouracil
  modified_base 2327: mod_base = OTHER; note = N1-methylpseudouracil
  modified_base 2328: mod_base = OTHER; note = N1-methylpseudouracil
  modified_base 2330: mod_base = OTHER; note = N1-methylpseudouracil
  modified_base 2331: mod_base = OTHER; note = N1-methylpseudouracil
  modified_base 2337: mod_base = OTHER; note = N1-methylpseudouracil
  modified_base 2338: mod_base = OTHER; note = N1-methylpseudouracil
  modified_base 2344: mod_base = OTHER; note = N1-methylpseudouracil
  modified_base 2357: mod_base = OTHER; note = N1-methylpseudouracil
  modified_base 2360: mod_base = OTHER; note = N1-methylpseudouracil
  modified_base 2365: mod_base = OTHER; note = N1-methylpseudouracil
  modified_base 2366: mod_base = OTHER; note = N1-methylpseudouracil
  modified_base 2369: mod_base = OTHER; note = N1-methylpseudouracil
  modified_base 2377: mod_base = OTHER; note = N1-methylpseudouracil
  modified_base 2385: mod_base = OTHER; note = N1-methylpseudouracil
  modified_base 2388: mod_base = OTHER; note = N1-methylpseudouracil
  modified_base 2390: mod_base = OTHER; note = N1-methylpseudouracil
  modified_base 2391: mod_base = OTHER; note = N1-methylpseudouracil
  modified_base 2392: mod_base = OTHER; note = N1-methylpseudouracil
  modified_base 2396: mod_base = OTHER; note = N1-methylpseudouracil
  modified_base 2401: mod_base = OTHER; note = N1-methylpseudouracil
  modified_base 2403: mod_base = OTHER; note = N1-methylpseudouracil
  modified_base 2407: mod_base = OTHER; note = N1-methylpseudouracil
gggaaanaagagagaaaagaagagnaagaagaaananaagaccccggcgccgccaccangaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacancngnacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngaccnganaanaggcnggagccncggnggccnagcnncnngccccnngggccnccccccagccccnccnccccnnccngcacccgnacccccgnggncnnngaanaaagncngagngggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 31 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 32 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 33 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 34 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 35 - protein, 153 aa, Homo sapiens
MYRMQLLSCIALSLALVTNSAPTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 36 - RNA, 2238 nt, synthetic construct
  source 1..2238: mol_type = other RNA
  modified_base 2: mod_base = OTHER; note = N1-methylpseudouracil
  modified_base 7: mod_base = OTHER; note = N1-methylpseudouracil
  modified_base 11: mod_base = OTHER; note = N1-methylpseudouracil
  modified_base 16: mod_base = OTHER; note = N1-methylpseudouracil
  modified_base 17: mod_base = OTHER; note = N1-methylpseudouracil
  modified_base 20: mod_base = OTHER; note = N1-methylpseudouracil
  modified_base 26: mod_base = OTHER; note = N1-methylpseudouracil
  modified_base 29: mod_base = OTHER; note = N1-methylpseudouracil
  modified_base 31: mod_base = OTHER; note = N1-methylpseudouracil
  modified_base 32: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 38: mod_base = OTHER; note = N1-methylpseudouracil
  modified_base 49: mod_base = OTHER; note = N1-methylpseudouracil
  modified_base 62: mod_base = OTHER; note = N1-methylpseudouracil
  modified_base 64: mod_base = OTHER; note = N1-methylpseudouracil
  modified_base 65: mod_base = OTHER; note = N1-methylpseudouracil
  modified_base 92: mod_base = OTHER; note = N1-methylpseudouracil
  modified_base 103: mod_base = OTHER; note = N1-methylpseudouracil
  modified_base 104: mod_base = OTHER; note = N1-methylpseudouracil
  modified_base 113: mod_base = OTHER; note = N1-methylpseudouracil
  modified_base 127: mod_base = OTHER; note = N1-methylpseudouracil
  modified_base 128: mod_base = OTHER; note = N1-methylpseudouracil
  modified_base 137: mod_base = OTHER; note = N1-methylpseudouracil
  modified_base 140: mod_base = OTHER; note = N1-methylpseudouracil
  modified_base 143: mod_base = OTHER; note = N1-methylpseudouracil
  modified_base 146: mod_base = OTHER; note = N1-methylpseudouracil
  modified_base 151: mod_base = OTHER; note = N1-methylpseudouracil
  modified_base 152: mod_base = OTHER; note = N1-methylpseudouracil
  modified_base 160: mod_base = OTHER; note = N1-methylpseudouracil
  modified_base 164: mod_base = OTHER; note = N1-methylpseudouracil
  modified_base 172: mod_base = OTHER; note = N1-methylpseudouracil
  modified_base 177: mod_base = OTHER; note = N1-methylpseudouracil
  modified_base 178: mod_base = OTHER; note = N1-methylpseudouracil
  modified_base 179: mod_base = OTHER; note = N1-methylpseudouracil
  modified_base 191: mod_base = OTHER; note = N1-methylpseudouracil
  modified_base 197: mod_base = OTHER; note = N1-methylpseudouracil
  modified_base 200: mod_base = OTHER; note = N1-methylpseudouracil
  modified_base 209: mod_base = OTHER; note = N1-methylpseudouracil
  modified_base 217: mod_base = OTHER; note = N1-methylpseudouracil
  modified_base 218: mod_base = OTHER; note = N1-methylpseudouracil
  modified_base 229: mod_base = OTHER; note = N1-methylpseudouracil
  modified_base 233: mod_base = OTHER; note = N1-methylpseudouracil
  modified_base 256: mod_base = OTHER; note = N1-methylpseudouracil
  modified_base 269: mod_base = OTHER; note = N1-methylpseudouracil
  modified_base 278: mod_base = OTHER; note = N1-methylpseudouracil
  modified_base 280: mod_base = OTHER; note = N1-methylpseudouracil
  modified_base 281: mod_base = OTHER; note = N1-methylpseudouracil
  modified_base 293: mod_base = OTHER; note = N1-methylpseudouracil
  modified_base 295: mod_base = OTHER; note = N1-methylpseudouracil
  modified_base 302: mod_base = OTHER; note = N1-methylpseudouracil
  modified_base 311: mod_base = OTHER; note = N1-methylpseudouracil
  modified_base 321: mod_base = OTHER; note = N1-methylpseudouracil
  modified_base 322: mod_base = OTHER; note = N1-methylpseudouracil
  modified_base 332: mod_base = OTHER; note = N1-methylpseudouracil
  modified_base 340: mod_base = OTHER; note = N1-methylpseudouracil
  modified_base 343: mod_base = OTHER; note = N1-methylpseudouracil
  modified_base 373: mod_base = OTHER; note = N1-methylpseudouracil
  modified_base 376: mod_base = OTHER; note = N1-methylpseudouracil
  modified_base 377: mod_base = OTHER; note = N1-methylpseudouracil
  modified_base 380: mod_base = OTHER; note = N1-methylpseudouracil
  modified_base 402: mod_base = OTHER; note = N1-methylpseudouracil
  modified_base 407: mod_base = OTHER; note = N1-methylpseudouracil
  modified_base 411: mod_base = OTHER; note = N1-methylpseudouracil
  modified_base 416: mod_base = OTHER; note = N1-methylpseudouracil
  modified_base 419: mod_base = OTHER; note = N1-methylpseudouracil
  modified_base 426: mod_base = OTHER; note = N1-methylpseudouracil
  modified_base 431: mod_base = OTHER; note = N1-methylpseudouracil
  modified_base 437: mod_base = OTHER; note = N1-methylpseudouracil
  modified_base 440: mod_base = OTHER; note = N1-methylpseudouracil
  modified_base 442: mod_base = OTHER; note = N1-methylpseudouracil
  modified_base 444: mod_base = OTHER; note = N1-methylpseudouracil
  modified_base 451: mod_base = OTHER; note = N1-methylpseudouracil
  modified_base 452: mod_base = OTHER; note = N1-methylpseudouracil
  modified_base 472: mod_base = OTHER; note = N1-methylpseudouracil
  modified_base 473: mod_base = OTHER; note = N1-methylpseudouracil
  modified_base 476: mod_base = OTHER; note = N1-methylpseudouracil
  modified_base 484: mod_base = OTHER; note = N1-methylpseudouracil
  modified_base 488: mod_base = OTHER; note = N1-methylpseudouracil
  modified_base 490: mod_base = OTHER; note = N1-methylpseudouracil
  modified_base 497: mod_base = OTHER; note = N1-methylpseudouracil
  modified_base 513: mod_base = OTHER; note = N1-methylpseudouracil
  modified_base 514: mod_base = OTHER; note = N1-methylpseudouracil
  modified_base 517: mod_base = OTHER; note = N1-methylpseudouracil
  modified_base 518: mod_base = OTHER; note = N1-methylpseudouracil
  modified_base 520: mod_base = OTHER; note = N1-methylpseudouracil
  modified_base 528: mod_base = OTHER; note = N1-methylpseudouracil
  modified_base 532: mod_base = OTHER; note = N1-methylpseudouracil
  modified_base 533: mod_base = OTHER; note = N1-methylpseudouracil
  modified_base 536: mod_base = OTHER; note = N1-methylpseudouracil
  modified_base 538: mod_base = OTHER; note = N1-methylpseudouracil
  modified_base 539: mod_base = OTHER; note = N1-methylpseudouracil
  modified_base 541: mod_base = OTHER; note = N1-methylpseudouracil
  modified_base 542: mod_base = OTHER; note = N1-methylpseudouracil
  modified_base 553: mod_base = OTHER; note = N1-methylpseudouracil
  modified_base 565: mod_base = OTHER; note = N1-methylpseudouracil
  modified_base 566: mod_base = OTHER; note = N1-methylpseudouracil
  modified_base 574: mod_base = OTHER; note = N1-methylpseudouracil
  modified_base 577: mod_base = OTHER; note = N1-methylpseudouracil
  modified_base 591: mod_base = OTHER; note = N1-methylpseudouracil
  modified_base 601: mod_base = OTHER; note = N1-methylpseudouracil
  modified_base 605: mod_base = OTHER; note = N1-methylpseudouracil
  modified_base 608: mod_base = OTHER; note = N1-methylpseudouracil
  modified_base 612: mod_base = OTHER; note = N1-methylpseudouracil
  modified_base 617: mod_base = OTHER; note = N1-methylpseudouracil
  modified_base 626: mod_base = OTHER; note = N1-methylpseudouracil
  modified_base 646: mod_base = OTHER; note = N1-methylpseudouracil
  modified_base 654: mod_base = OTHER; note = N1-methylpseudouracil
  modified_base 665: mod_base = OTHER; note = N1-methylpseudouracil
  modified_base 670: mod_base = OTHER; note = N1-methylpseudouracil
  modified_base 680: mod_base = OTHER; note = N1-methylpseudouracil
  modified_base 688: mod_base = OTHER; note = N1-methylpseudouracil
  modified_base 689: mod_base = OTHER; note = N1-methylpseudouracil
  modified_base 702: mod_base = OTHER; note = N1-methylpseudouracil
  modified_base 703: mod_base = OTHER; note = N1-methylpseudouracil
  modified_base 704: mod_base = OTHER; note = N1-methylpseudouracil
  modified_base 712: mod_base = OTHER; note = N1-methylpseudouracil
  modified_base 719: mod_base = OTHER; note = N1-methylpseudouracil
  modified_base 723: mod_base = OTHER; note = N1-methylpseudouracil
  modified_base 727: mod_base = OTHER; note = N1-methylpseudouracil
  modified_base 728: mod_base = OTHER; note = N1-methylpseudouracil
  modified_base 739: mod_base = OTHER; note = N1-methylpseudouracil
  modified_base 740: mod_base = OTHER; note = N1-methylpseudouracil
  modified_base 744: mod_base = OTHER; note = N1-methylpseudouracil
  modified_base 754: mod_base = OTHER; note = N1-methylpseudouracil
  modified_base 755: mod_base = OTHER; note = N1-methylpseudouracil
  modified_base 759: mod_base = OTHER; note = N1-methylpseudouracil
  modified_base 764: mod_base = OTHER; note = N1-methylpseudouracil
  modified_base 773: mod_base = OTHER; note = N1-methylpseudouracil
  modified_base 776: mod_base = OTHER; note = N1-methylpseudouracil
  modified_base 780: mod_base = OTHER; note = N1-methylpseudouracil
  modified_base 785: mod_base = OTHER; note = N1-methylpseudouracil
  modified_base 794: mod_base = OTHER; note = N1-methylpseudouracil
  modified_base 798: mod_base = OTHER; note = N1-methylpseudouracil
  modified_base 805: mod_base = OTHER; note = N1-methylpseudouracil
  modified_base 808: mod_base = OTHER; note = N1-methylpseudouracil
  modified_base 810: mod_base = OTHER; note = N1-methylpseudouracil
  modified_base 821: mod_base = OTHER; note = N1-methylpseudouracil
  modified_base 824: mod_base = OTHER; note = N1-methylpseudouracil
  modified_base 829: mod_base = OTHER; note = N1-methylpseudouracil
  modified_base 851: mod_base = OTHER; note = N1-methylpseudouracil
  modified_base 859: mod_base = OTHER; note = N1-methylpseudouracil
  modified_base 863: mod_base = OTHER; note = N1-methylpseudouracil
  modified_base 865: mod_base = OTHER; note = N1-methylpseudouracil
  modified_base 884: mod_base = OTHER; note = N1-methylpseudouracil
  modified_base 896: mod_base = OTHER; note = N1-methylpseudouracil
  modified_base 904: mod_base = OTHER; note = N1-methylpseudouracil
  modified_base 906: mod_base = OTHER; note = N1-methylpseudouracil
  modified_base 907: mod_base = OTHER; note = N1-methylpseudouracil
  modified_base 909: mod_base = OTHER; note = N1-methylpseudouracil
  modified_base 918: mod_base = OTHER; note = N1-methylpseudouracil
  modified_base 920: mod_base = OTHER; note = N1-methylpseudouracil
  modified_base 922: mod_base = OTHER; note = N1-methylpseudouracil
  modified_base 923: mod_base = OTHER; note = N1-methylpseudouracil
  modified_base 933: mod_base = OTHER; note = N1-methylpseudouracil
  modified_base 937: mod_base = OTHER; note = N1-methylpseudouracil
  modified_base 941: mod_base = OTHER; note = N1-methylpseudouracil
  modified_base 942: mod_base = OTHER; note = N1-methylpseudouracil
  modified_base 950: mod_base = OTHER; note = N1-methylpseudouracil
  modified_base 965: mod_base = OTHER; note = N1-methylpseudouracil
  modified_base 969: mod_base = OTHER; note = N1-methylpseudouracil
  modified_base 975: mod_base = OTHER; note = N1-methylpseudouracil
  modified_base 977: mod_base = OTHER; note = N1-methylpseudouracil
  modified_base 985: mod_base = OTHER; note = N1-methylpseudouracil
  modified_base 986: mod_base = OTHER; note = N1-methylpseudouracil
  modified_base 997: mod_base = OTHER; note = N1-methylpseudouracil
  modified_base 998: mod_base = OTHER; note = N1-methylpseudouracil
  modified_base 1001: mod_base = OTHER; note = N1-methylpseudouracil
  modified_base 1016: mod_base = OTHER; note = N1-methylpseudouracil
  modified_base 1018: mod_base = OTHER; note = N1-methylpseudouracil
  modified_base 1027: mod_base = OTHER; note = N1-methylpseudouracil
  modified_base 1046: mod_base = OTHER; note = N1-methylpseudouracil
  modified_base 1047: mod_base = OTHER; note = N1-methylpseudouracil
  modified_base 1048: mod_base = OTHER; note = N1-methylpseudouracil
  modified_base 1049: mod_base = OTHER; note = N1-methylpseudouracil
  modified_base 1052: mod_base = OTHER; note = N1-methylpseudouracil
  modified_base 1058: mod_base = OTHER; note = N1-methylpseudouracil
  modified_base 1060: mod_base = OTHER; note = N1-methylpseudouracil
  modified_base 1061: mod_base = OTHER; note = N1-methylpseudouracil
  modified_base 1064: mod_base = OTHER; note = N1-methylpseudouracil
  modified_base 1066: mod_base = OTHER; note = N1-methylpseudouracil
  modified_base 1072: mod_base = OTHER; note = N1-methylpseudouracil
  modified_base 1086: mod_base = OTHER; note = N1-methylpseudouracil
  modified_base 1093: mod_base = OTHER; note = N1-methylpseudouracil
  modified_base 1100: mod_base = OTHER; note = N1-methylpseudouracil
  modified_base 1103: mod_base = OTHER; note = N1-methylpseudouracil
  modified_base 1106: mod_base = OTHER; note = N1-methylpseudouracil
  modified_base 1109: mod_base = OTHER; note = N1-methylpseudouracil
  modified_base 1112: mod_base = OTHER; note = N1-methylpseudouracil
  modified_base 1118: mod_base = OTHER; note = N1-methylpseudouracil
  modified_base 1122: mod_base = OTHER; note = N1-methylpseudouracil
  modified_base 1129: mod_base = OTHER; note = N1-methylpseudouracil
  modified_base 1137: mod_base = OTHER; note = N1-methylpseudouracil
  modified_base 1142: mod_base = OTHER; note = N1-methylpseudouracil
  modified_base 1150: mod_base = OTHER; note = N1-methylpseudouracil
  modified_base 1153: mod_base = OTHER; note = N1-methylpseudouracil
  modified_base 1170: mod_base = OTHER; note = N1-methylpseudouracil
  modified_base 1177: mod_base = OTHER; note = N1-methylpseudouracil
  modified_base 1179: mod_base = OTHER; note = N1-methylpseudouracil
  modified_base 1180: mod_base = OTHER; note = N1-methylpseudouracil
  modified_base 1190: mod_base = OTHER; note = N1-methylpseudouracil
  modified_base 1192: mod_base = OTHER; note = N1-methylpseudouracil
  modified_base 1193: mod_base = OTHER; note = N1-methylpseudouracil
  modified_base 1201: mod_base = OTHER; note = N1-methylpseudouracil
  modified_base 1202: mod_base = OTHER; note = N1-methylpseudouracil
  modified_base 1209: mod_base = OTHER; note = N1-methylpseudouracil
  modified_base 1211: mod_base = OTHER; note = N1-methylpseudouracil
  modified_base 1214: mod_base = OTHER; note = N1-methylpseudouracil
  modified_base 1224: mod_base = OTHER; note = N1-methylpseudouracil
  modified_base 1232: mod_base = OTHER; note = N1-methylpseudouracil
  modified_base 1235: mod_base = OTHER; note = N1-methylpseudouracil
  modified_base 1246: mod_base = OTHER; note = N1-methylpseudouracil
  modified_base 1248: mod_base = OTHER; note = N1-methylpseudouracil
  modified_base 1253: mod_base = OTHER; note = N1-methylpseudouracil
  modified_base 1255: mod_base = OTHER; note = N1-methylpseudouracil
  modified_base 1256: mod_base = OTHER; note = N1-methylpseudouracil
  modified_base 1265: mod_base = OTHER; note = N1-methylpseudouracil
  modified_base 1273: mod_base = OTHER; note = N1-methylpseudouracil
  modified_base 1279: mod_base = OTHER; note = N1-methylpseudouracil
  modified_base 1280: mod_base = OTHER; note = N1-methylpseudouracil
  modified_base 1292: mod_base = OTHER; note = N1-methylpseudouracil
  modified_base 1294: mod_base = OTHER; note = N1-methylpseudouracil
  modified_base 1295: mod_base = OTHER; note = N1-methylpseudouracil
  modified_base 1298: mod_base = OTHER; note = N1-methylpseudouracil
  modified_base 1303: mod_base = OTHER; note = N1-methylpseudouracil
  modified_base 1316: mod_base = OTHER; note = N1-methylpseudouracil
  modified_base 1320: mod_base = OTHER; note = N1-methylpseudouracil
  modified_base 1325: mod_base = OTHER; note = N1-methylpseudouracil
  modified_base 1329: mod_base = OTHER; note = N1-methylpseudouracil
  modified_base 1335: mod_base = OTHER; note = N1-methylpseudouracil
  modified_base 1340: mod_base = OTHER; note = N1-methylpseudouracil
  modified_base 1343: mod_base = OTHER; note = N1-methylpseudouracil
  modified_base 1349: mod_base = OTHER; note = N1-methylpseudouracil
  modified_base 1350: mod_base = OTHER; note = N1-methylpseudouracil
  modified_base 1353: mod_base = OTHER; note = N1-methylpseudouracil
  modified_base 1361: mod_base = OTHER; note = N1-methylpseudouracil
  modified_base 1370: mod_base = OTHER; note = N1-methylpseudouracil
  modified_base 1381: mod_base = OTHER; note = N1-methylpseudouracil
  modified_base 1384: mod_base = OTHER; note = N1-methylpseudouracil
  modified_base 1386: mod_base = OTHER; note = N1-methylpseudouracil
  modified_base 1401: mod_base = OTHER; note = N1-methylpseudouracil
  modified_base 1409: mod_base = OTHER; note = N1-methylpseudouracil
  modified_base 1413: mod_base = OTHER; note = N1-methylpseudouracil
  modified_base 1414: mod_base = OTHER; note = N1-methylpseudouracil
  modified_base 1426: mod_base = OTHER; note = N1-methylpseudouracil
  modified_base 1430: mod_base = OTHER; note = N1-methylpseudouracil
  modified_base 1432: mod_base = OTHER; note = N1-methylpseudouracil
  modified_base 1436: mod_base = OTHER; note = N1-methylpseudouracil
  modified_base 1437: mod_base = OTHER; note = N1-methylpseudouracil
  modified_base 1439: mod_base = OTHER; note = N1-methylpseudouracil
  modified_base 1442: mod_base = OTHER; note = N1-methylpseudouracil
  modified_base 1451: mod_base = OTHER; note = N1-methylpseudouracil
  modified_base 1453: mod_base = OTHER; note = N1-methylpseudouracil
  modified_base 1457: mod_base = OTHER; note = N1-methylpseudouracil
  modified_base 1460: mod_base = OTHER; note = N1-methylpseudouracil
  modified_base 1476: mod_base = OTHER; note = N1-methylpseudouracil
  modified_base 1478: mod_base = OTHER; note = N1-methylpseudouracil
  modified_base 1490: mod_base = OTHER; note = N1-methylpseudouracil
  modified_base 1498: mod_base = OTHER; note = N1-methylpseudouracil
  modified_base 1500: mod_base = OTHER; note = N1-methylpseudouracil
  modified_base 1501: mod_base = OTHER; note = N1-methylpseudouracil
  modified_base 1513: mod_base = OTHER; note = N1-methylpseudouracil
  modified_base 1514: mod_base = OTHER; note = N1-methylpseudouracil
  modified_base 1517: mod_base = OTHER; note = N1-methylpseudouracil
  modified_base 1521: mod_base = OTHER; note = N1-methylpseudouracil
  modified_base 1530: mod_base = OTHER; note = N1-methylpseudouracil
  modified_base 1531: mod_base = OTHER; note = N1-methylpseudouracil
  modified_base 1534: mod_base = OTHER; note = N1-methylpseudouracil
  modified_base 1535: mod_base = OTHER; note = N1-methylpseudouracil
  modified_base 1544: mod_base = OTHER; note = N1-methylpseudouracil
  modified_base 1550: mod_base = OTHER; note = N1-methylpseudouracil
  modified_base 1551: mod_base = OTHER; note = N1-methylpseudouracil
  modified_base 1560: mod_base = OTHER; note = N1-methylpseudouracil
  modified_base 1561: mod_base = OTHER; note = N1-methylpseudouracil
  modified_base 1565: mod_base = OTHER; note = N1-methylpseudouracil
  modified_base 1569: mod_base = OTHER; note = N1-methylpseudouracil
  modified_base 1576: mod_base = OTHER; note = N1-methylpseudouracil
  modified_base 1577: mod_base = OTHER; note = N1-methylpseudouracil
  modified_base 1590: mod_base = OTHER; note = N1-methylpseudouracil
  modified_base 1591: mod_base = OTHER; note = N1-methylpseudouracil
  modified_base 1592: mod_base = OTHER; note = N1-methylpseudouracil
  modified_base 1597: mod_base = OTHER; note = N1-methylpseudouracil
  modified_base 1598: mod_base = OTHER; note = N1-methylpseudouracil
  modified_base 1610: mod_base = OTHER; note = N1-methylpseudouracil
  modified_base 1611: mod_base = OTHER; note = N1-methylpseudouracil
  modified_base 1612: mod_base = OTHER; note = N1-methylpseudouracil
  modified_base 1617: mod_base = OTHER; note = N1-methylpseudouracil
  modified_base 1619: mod_base = OTHER; note = N1-methylpseudouracil
  modified_base 1640: mod_base = OTHER; note = N1-methylpseudouracil
  modified_base 1658: mod_base = OTHER; note = N1-methylpseudouracil
  modified_base 1661: mod_base = OTHER; note = N1-methylpseudouracil
  modified_base 1666: mod_base = OTHER; note = N1-methylpseudouracil
  modified_base 1667: mod_base = OTHER; note = N1-methylpseudouracil
  modified_base 1670: mod_base = OTHER; note = N1-methylpseudouracil
  modified_base 1703: mod_base = OTHER; note = N1-methylpseudouracil
  modified_base 1704: mod_base = OTHER; note = N1-methylpseudouracil
  modified_base 1712: mod_base = OTHER; note = N1-methylpseudouracil
  modified_base 1715: mod_base = OTHER; note = N1-methylpseudouracil
  modified_base 1723: mod_base = OTHER; note = N1-methylpseudouracil
  modified_base 1724: mod_base = OTHER; note = N1-methylpseudouracil
  modified_base 1732: mod_base = OTHER; note = N1-methylpseudouracil
  modified_base 1733: mod_base = OTHER; note = N1-methylpseudouracil
  modified_base 1736: mod_base = OTHER; note = N1-methylpseudouracil
  modified_base 1744: mod_base = OTHER; note = N1-methylpseudouracil
  modified_base 1746: mod_base = OTHER; note = N1-methylpseudouracil
  modified_base 1747: mod_base = OTHER; note = N1-methylpseudouracil
  modified_base 1749: mod_base = OTHER; note = N1-methylpseudouracil
  modified_base 1770: mod_base = OTHER; note = N1-methylpseudouracil
  modified_base 1771: mod_base = OTHER; note = N1-methylpseudouracil
  modified_base 1774: mod_base = OTHER; note = N1-methylpseudouracil
  modified_base 1775: mod_base = OTHER; note = N1-methylpseudouracil
  modified_base 1795: mod_base = OTHER; note = N1-methylpseudouracil
  modified_base 1796: mod_base = OTHER; note = N1-methylpseudouracil
  modified_base 1799: mod_base = OTHER; note = N1-methylpseudouracil
  modified_base 1807: mod_base = OTHER; note = N1-methylpseudouracil
  modified_base 1809: mod_base = OTHER; note = N1-methylpseudouracil
  modified_base 1815: mod_base = OTHER; note = N1-methylpseudouracil
  modified_base 1819: mod_base = OTHER; note = N1-methylpseudouracil
  modified_base 1820: mod_base = OTHER; note = N1-methylpseudouracil
  modified_base 1826: mod_base = OTHER; note = N1-methylpseudouracil
  modified_base 1830: mod_base = OTHER; note = N1-methylpseudouracil
  modified_base 1845: mod_base = OTHER; note = N1-methylpseudouracil
  modified_base 1855: mod_base = OTHER; note = N1-methylpseudouracil
  modified_base 1874: mod_base = OTHER; note = N1-methylpseudouracil
  modified_base 1880: mod_base = OTHER; note = N1-methylpseudouracil
  modified_base 1889: mod_base = OTHER; note = N1-methylpseudouracil
  modified_base 1892: mod_base = OTHER; note = N1-methylpseudouracil
  modified_base 1895: mod_base = OTHER; note = N1-methylpseudouracil
  modified_base 1900: mod_base = OTHER; note = N1-methylpseudouracil
  modified_base 1901: mod_base = OTHER; note = N1-methylpseudouracil
  modified_base 1907: mod_base = OTHER; note = N1-methylpseudouracil
  modified_base 1910: mod_base = OTHER; note = N1-methylpseudouracil
  modified_base 1913: mod_base = OTHER; note = N1-methylpseudouracil
  modified_base 1922: mod_base = OTHER; note = N1-methylpseudouracil
  modified_base 1930: mod_base = OTHER; note = N1-methylpseudouracil
  modified_base 1946: mod_base = OTHER; note = N1-methylpseudouracil
  modified_base 1955: mod_base = OTHER; note = N1-methylpseudouracil
  modified_base 1958: mod_base = OTHER; note = N1-methylpseudouracil
  modified_base 1963: mod_base = OTHER; note = N1-methylpseudouracil
  modified_base 1964: mod_base = OTHER; note = N1-methylpseudouracil
  modified_base 1969: mod_base = OTHER; note = N1-methylpseudouracil
  modified_base 1970: mod_base = OTHER; note = N1-methylpseudouracil
  modified_base 1972: mod_base = OTHER; note = N1-methylpseudouracil
  modified_base 1976: mod_base = OTHER; note = N1-methylpseudouracil
  modified_base 1980: mod_base = OTHER; note = N1-methylpseudouracil
  modified_base 1992: mod_base = OTHER; note = N1-methylpseudouracil
  modified_base 1997: mod_base = OTHER; note = N1-methylpseudouracil
  modified_base 2006: mod_base = OTHER; note = N1-methylpseudouracil
  modified_base 2007: mod_base = OTHER; note = N1-methylpseudouracil
  modified_base 2011: mod_base = OTHER; note = N1-methylpseudouracil
  modified_base 2015: mod_base = OTHER; note = N1-methylpseudouracil
  modified_base 2027: mod_base = OTHER; note = N1-methylpseudouracil
  modified_base 2028: mod_base = OTHER; note = N1-methylpseudouracil
  modified_base 2036: mod_base = OTHER; note = N1-methylpseudouracil
  modified_base 2048: mod_base = OTHER; note = N1-methylpseudouracil
  modified_base 2054: mod_base = OTHER; note = N1-methylpseudouracil
  modified_base 2061: mod_base = OTHER; note = N1-methylpseudouracil
  modified_base 2064: mod_base = OTHER; note = N1-methylpseudouracil
  modified_base 2070: mod_base = OTHER; note = N1-methylpseudouracil
  modified_base 2071: mod_base = OTHER; note = N1-methylpseudouracil
  modified_base 2072: mod_base = OTHER; note = N1-methylpseudouracil
  modified_base 2078: mod_base = OTHER; note = N1-methylpseudouracil
  modified_base 2085: mod_base = OTHER; note = N1-methylpseudouracil
  modified_base 2093: mod_base = OTHER; note = N1-methylpseudouracil
  modified_base 2094: mod_base = OTHER; note = N1-methylpseudouracil
  modified_base 2096: mod_base = OTHER; note = N1-methylpseudouracil
  modified_base 2097: mod_base = OTHER; note = N1-methylpseudouracil
  modified_base 2105: mod_base = OTHER; note = N1-methylpseudouracil
  modified_base 2111: mod_base = OTHER; note = N1-methylpseudouracil
  modified_base 2114: mod_base = OTHER; note = N1-methylpseudouracil
  modified_base 2117: mod_base = OTHER; note = N1-methylpseudouracil
  modified_base 2120: mod_base = OTHER; note = N1-methylpseudouracil
  modified_base 2125: mod_base = OTHER; note = N1-methylpseudouracil
  modified_base 2126: mod_base = OTHER; note = N1-methylpseudouracil
  modified_base 2134: mod_base = OTHER; note = N1-methylpseudouracil
  modified_base 2146: mod_base = OTHER; note = N1-methylpseudouracil
  modified_base 2147: mod_base = OTHER; note = N1-methylpseudouracil
  modified_base 2150: mod_base = OTHER; note = N1-methylpseudouracil
  modified_base 2152: mod_base = OTHER; note = N1-methylpseudouracil
  modified_base 2158: mod_base = OTHER; note = N1-methylpseudouracil
  modified_base 2180: mod_base = OTHER; note = N1-methylpseudouracil
  modified_base 2183: mod_base = OTHER; note = N1-methylpseudouracil
  modified_base 2188: mod_base = OTHER; note = N1-methylpseudouracil
  modified_base 2189: mod_base = OTHER; note = N1-methylpseudouracil
  modified_base 2192: mod_base = OTHER; note = N1-methylpseudouracil
  modified_base 2200: mod_base = OTHER; note = N1-methylpseudouracil
  modified_base 2204: mod_base = OTHER; note = N1-methylpseudouracil
  modified_base 2209: mod_base = OTHER; note = N1-methylpseudouracil
  modified_base 2210: mod_base = OTHER; note = N1-methylpseudouracil
  modified_base 2212: mod_base = OTHER; note = N1-methylpseudouracil
  modified_base 2222: mod_base = OTHER; note = N1-methylpseudouracil
  modified_base 2225: mod_base = OTHER; note = N1-methylpseudouracil
  modified_base 2227: mod_base = OTHER; note = N1-methylpseudouracil
  modified_base 2229: mod_base = OTHER; note = N1-methylpseudouracil
  modified_base 2232: mod_base = OTHER; note = N1-methylpseudouracil
  modified_base 2234: mod_base = OTHER; note = N1-methylpseudouracil
angaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacannngcacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngacc

SEQ ID NO: 37 - RNA, 2514 nt, synthetic construct
  source 1..2514: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 59: mod_base = OTHER; note = N1-methylpseudouracil
  modified_base 64: mod_base = OTHER; note = N1-methylpseudouracil
  modified_base 68: mod_base = OTHER; note = N1-methylpseudouracil
  modified_base 73: mod_base = OTHER; note = N1-methylpseudouracil
  modified_base 74: mod_base = OTHER; note = N1-methylpseudouracil
  modified_base 77: mod_base = OTHER; note = N1-methylpseudouracil
  modified_base 83: mod_base = OTHER; note = N1-methylpseudouracil
  modified_base 86: mod_base = OTHER; note = N1-methylpseudouracil
  modified_base 88: mod_base = OTHER; note = N1-methylpseudouracil
  modified_base 89: mod_base = OTHER; note = N1-methylpseudouracil
  modified_base 92: mod_base = OTHER; note = N1-methylpseudouracil
  modified_base 94: mod_base = OTHER; note = N1-methylpseudouracil
  modified_base 95: mod_base = OTHER; note = N1-methylpseudouracil
  modified_base 106: mod_base = OTHER; note = N1-methylpseudouracil
  modified_base 119: mod_base = OTHER; note = N1-methylpseudouracil
  modified_base 121: mod_base = OTHER; note = N1-methylpseudouracil
  modified_base 122: mod_base = OTHER; note = N1-methylpseudouracil
  modified_base 149: mod_base = OTHER; note = N1-methylpseudouracil
  modified_base 160: mod_base = OTHER; note = N1-methylpseudouracil
  modified_base 161: mod_base = OTHER; note = N1-methylpseudouracil
  modified_base 170: mod_base = OTHER; note = N1-methylpseudouracil
  modified_base 184: mod_base = OTHER; note = N1-methylpseudouracil
  modified_base 185: mod_base = OTHER; note = N1-methylpseudouracil
  modified_base 194: mod_base = OTHER; note = N1-methylpseudouracil
  modified_base 197: mod_base = OTHER; note = N1-methylpseudouracil
  modified_base 200: mod_base = OTHER; note = N1-methylpseudouracil
  modified_base 203: mod_base = OTHER; note = N1-methylpseudouracil
  modified_base 208: mod_base = OTHER; note = N1-methylpseudouracil
  modified_base 209: mod_base = OTHER; note = N1-methylpseudouracil
  modified_base 217: mod_base = OTHER; note = N1-methylpseudouracil
  modified_base 221: mod_base = OTHER; note = N1-methylpseudouracil
  modified_base 229: mod_base = OTHER; note = N1-methylpseudouracil
  modified_base 234: mod_base = OTHER; note = N1-methylpseudouracil
  modified_base 235: mod_base = OTHER; note = N1-methylpseudouracil
  modified_base 236: mod_base = OTHER; note = N1-methylpseudouracil
  modified_base 248: mod_base = OTHER; note = N1-methylpseudouracil
  modified_base 254: mod_base = OTHER; note = N1-methylpseudouracil
  modified_base 257: mod_base = OTHER; note = N1-methylpseudouracil
  modified_base 266: mod_base = OTHER; note = N1-methylpseudouracil
  modified_base 274: mod_base = OTHER; note = N1-methylpseudouracil
  modified_base 275: mod_base = OTHER; note = N1-methylpseudouracil
  modified_base 286: mod_base = OTHER; note = N1-methylpseudouracil
  modified_base 290: mod_base = OTHER; note = N1-methylpseudouracil
  modified_base 313: mod_base = OTHER; note = N1-methylpseudouracil
  modified_base 326: mod_base = OTHER; note = N1-methylpseudouracil
  modified_base 335: mod_base = OTHER; note = N1-methylpseudouracil
  modified_base 337: mod_base = OTHER; note = N1-methylpseudouracil
  modified_base 338: mod_base = OTHER; note = N1-methylpseudouracil
  modified_base 350: mod_base = OTHER; note = N1-methylpseudouracil
  modified_base 352: mod_base = OTHER; note = N1-methylpseudouracil
  modified_base 359: mod_base = OTHER; note = N1-methylpseudouracil
  modified_base 368: mod_base = OTHER; note = N1-methylpseudouracil
  modified_base 378: mod_base = OTHER; note = N1-methylpseudouracil
  modified_base 379: mod_base = OTHER; note = N1-methylpseudouracil
  modified_base 389: mod_base = OTHER; note = N1-methylpseudouracil
  modified_base 397: mod_base = OTHER; note = N1-methylpseudouracil
  modified_base 400: mod_base = OTHER; note = N1-methylpseudouracil
  modified_base 430: mod_base = OTHER; note = N1-methylpseudouracil
  modified_base 433: mod_base = OTHER; note = N1-methylpseudouracil
  modified_base 434: mod_base = OTHER; note = N1-methylpseudouracil
  modified_base 437: mod_base = OTHER; note = N1-methylpseudouracil
  modified_base 459: mod_base = OTHER; note = N1-methylpseudouracil
  modified_base 464: mod_base = OTHER; note = N1-methylpseudouracil
  modified_base 468: mod_base = OTHER; note = N1-methylpseudouracil
  modified_base 473: mod_base = OTHER; note = N1-methylpseudouracil
  modified_base 476: mod_base = OTHER; note = N1-methylpseudouracil
  modified_base 483: mod_base = OTHER; note = N1-methylpseudouracil
  modified_base 488: mod_base = OTHER; note = N1-methylpseudouracil
  modified_base 494: mod_base = OTHER; note = N1-methylpseudouracil
  modified_base 497: mod_base = OTHER; note = N1-methylpseudouracil
  modified_base 499: mod_base = OTHER; note = N1-methylpseudouracil
  modified_base 501: mod_base = OTHER; note = N1-methylpseudouracil
  modified_base 508: mod_base = OTHER; note = N1-methylpseudouracil
  modified_base 509: mod_base = OTHER; note = N1-methylpseudouracil
  modified_base 529: mod_base = OTHER; note = N1-methylpseudouracil
  modified_base 530: mod_base = OTHER; note = N1-methylpseudouracil
  modified_base 533: mod_base = OTHER; note = N1-methylpseudouracil
  modified_base 541: mod_base = OTHER; note = N1-methylpseudouracil
  modified_base 545: mod_base = OTHER; note = N1-methylpseudouracil
  modified_base 547: mod_base = OTHER; note = N1-methylpseudouracil
  modified_base 554: mod_base = OTHER; note = N1-methylpseudouracil
  modified_base 570: mod_base = OTHER; note = N1-methylpseudouracil
  modified_base 571: mod_base = OTHER; note = N1-methylpseudouracil
  modified_base 574: mod_base = OTHER; note = N1-methylpseudouracil
  modified_base 575: mod_base = OTHER; note = N1-methylpseudouracil
  modified_base 577: mod_base = OTHER; note = N1-methylpseudouracil
  modified_base 585: mod_base = OTHER; note = N1-methylpseudouracil
  modified_base 589: mod_base = OTHER; note = N1-methylpseudouracil
  modified_base 590: mod_base = OTHER; note = N1-methylpseudouracil
  modified_base 593: mod_base = OTHER; note = N1-methylpseudouracil
  modified_base 595: mod_base = OTHER; note = N1-methylpseudouracil
  modified_base 596: mod_base = OTHER; note = N1-methylpseudouracil
  modified_base 598: mod_base = OTHER; note = N1-methylpseudouracil
  modified_base 599: mod_base = OTHER; note = N1-methylpseudouracil
  modified_base 610: mod_base = OTHER; note = N1-methylpseudouracil
  modified_base 622: mod_base = OTHER; note = N1-methylpseudouracil
  modified_base 623: mod_base = OTHER; note = N1-methylpseudouracil
  modified_base 631: mod_base = OTHER; note = N1-methylpseudouracil
  modified_base 634: mod_base = OTHER; note = N1-methylpseudouracil
  modified_base 648: mod_base = OTHER; note = N1-methylpseudouracil
  modified_base 658: mod_base = OTHER; note = N1-methylpseudouracil
  modified_base 662: mod_base = OTHER; note = N1-methylpseudouracil
  modified_base 665: mod_base = OTHER; note = N1-methylpseudouracil
  modified_base 669: mod_base = OTHER; note = N1-methylpseudouracil
  modified_base 674: mod_base = OTHER; note = N1-methylpseudouracil
  modified_base 683: mod_base = OTHER; note = N1-methylpseudouracil
  modified_base 703: mod_base = OTHER; note = N1-methylpseudouracil
  modified_base 711: mod_base = OTHER; note = N1-methylpseudouracil
  modified_base 722: mod_base = OTHER; note = N1-methylpseudouracil
  modified_base 727: mod_base = OTHER; note = N1-methylpseudouracil
  modified_base 737: mod_base = OTHER; note = N1-methylpseudouracil
  modified_base 745: mod_base = OTHER; note = N1-methylpseudouracil
  modified_base 746: mod_base = OTHER; note = N1-methylpseudouracil
  modified_base 759: mod_base = OTHER; note = N1-methylpseudouracil
  modified_base 760: mod_base = OTHER; note = N1-methylpseudouracil
  modified_base 761: mod_base = OTHER; note = N1-methylpseudouracil
  modified_base 769: mod_base = OTHER; note = N1-methylpseudouracil
  modified_base 776: mod_base = OTHER; note = N1-methylpseudouracil
  modified_base 780: mod_base = OTHER; note = N1-methylpseudouracil
  modified_base 784: mod_base = OTHER; note = N1-methylpseudouracil
  modified_base 785: mod_base = OTHER; note = N1-methylpseudouracil
  modified_base 796: mod_base = OTHER; note = N1-methylpseudouracil
  modified_base 797: mod_base = OTHER; note = N1-methylpseudouracil
  modified_base 801: mod_base = OTHER; note = N1-methylpseudouracil
  modified_base 811: mod_base = OTHER; note = N1-methylpseudouracil
  modified_base 812: mod_base = OTHER; note = N1-methylpseudouracil
  modified_base 816: mod_base = OTHER; note = N1-methylpseudouracil
  modified_base 821: mod_base = OTHER; note = N1-methylpseudouracil
  modified_base 830: mod_base = OTHER; note = N1-methylpseudouracil
  modified_base 833: mod_base = OTHER; note = N1-methylpseudouracil
  modified_base 837: mod_base = OTHER; note = N1-methylpseudouracil
  modified_base 842: mod_base = OTHER; note = N1-methylpseudouracil
  modified_base 851: mod_base = OTHER; note = N1-methylpseudouracil
  modified_base 855: mod_base = OTHER; note = N1-methylpseudouracil
  modified_base 862: mod_base = OTHER; note = N1-methylpseudouracil
  modified_base 865: mod_base = OTHER; note = N1-methylpseudouracil
  modified_base 867: mod_base = OTHER; note = N1-methylpseudouracil
  modified_base 878: mod_base = OTHER; note = N1-methylpseudouracil
  modified_base 881: mod_base = OTHER; note = N1-methylpseudouracil
  modified_base 886: mod_base = OTHER; note = N1-methylpseudouracil
  modified_base 908: mod_base = OTHER; note = N1-methylpseudouracil
  modified_base 916: mod_base = OTHER; note = N1-methylpseudouracil
  modified_base 920: mod_base = OTHER; note = N1-methylpseudouracil
  modified_base 922: mod_base = OTHER; note = N1-methylpseudouracil
  modified_base 941: mod_base = OTHER; note = N1-methylpseudouracil
  modified_base 953: mod_base = OTHER; note = N1-methylpseudouracil
  modified_base 961: mod_base = OTHER; note = N1-methylpseudouracil
  modified_base 963: mod_base = OTHER; note = N1-methylpseudouracil
  modified_base 964: mod_base = OTHER; note = N1-methylpseudouracil
  modified_base 966: mod_base = OTHER; note = N1-methylpseudouracil
  modified_base 975: mod_base = OTHER; note = N1-methylpseudouracil
  modified_base 977: mod_base = OTHER; note = N1-methylpseudouracil
  modified_base 979: mod_base = OTHER; note = N1-methylpseudouracil
  modified_base 980: mod_base = OTHER; note = N1-methylpseudouracil
  modified_base 990: mod_base = OTHER; note = N1-methylpseudouracil
  modified_base 994: mod_base = OTHER; note = N1-methylpseudouracil
  modified_base 998: mod_base = OTHER; note = N1-methylpseudouracil
  modified_base 999: mod_base = OTHER; note = N1-methylpseudouracil
  modified_base 1007: mod_base = OTHER; note = N1-methylpseudouracil
  modified_base 1022: mod_base = OTHER; note = N1-methylpseudouracil
  modified_base 1026: mod_base = OTHER; note = N1-methylpseudouracil
  modified_base 1032: mod_base = OTHER; note = N1-methylpseudouracil
  modified_base 1034: mod_base = OTHER; note = N1-methylpseudouracil
  modified_base 1042: mod_base = OTHER; note = N1-methylpseudouracil
  modified_base 1043: mod_base = OTHER; note = N1-methylpseudouracil
  modified_base 1054: mod_base = OTHER; note = N1-methylpseudouracil
  modified_base 1055: mod_base = OTHER; note = N1-methylpseudouracil
  modified_base 1058: mod_base = OTHER; note = N1-methylpseudouracil
  modified_base 1073: mod_base = OTHER; note = N1-methylpseudouracil
  modified_base 1075: mod_base = OTHER; note = N1-methylpseudouracil
  modified_base 1084: mod_base = OTHER; note = N1-methylpseudouracil
  modified_base 1103: mod_base = OTHER; note = N1-methylpseudouracil
  modified_base 1104: mod_base = OTHER; note = N1-methylpseudouracil
  modified_base 1105: mod_base = OTHER; note = N1-methylpseudouracil
  modified_base 1106: mod_base = OTHER; note = N1-methylpseudouracil
  modified_base 1109: mod_base = OTHER; note = N1-methylpseudouracil
  modified_base 1115: mod_base = OTHER; note = N1-methylpseudouracil
  modified_base 1117: mod_base = OTHER; note = N1-methylpseudouracil
  modified_base 1118: mod_base = OTHER; note = N1-methylpseudouracil
  modified_base 1121: mod_base = OTHER; note = N1-methylpseudouracil
  modified_base 1123: mod_base = OTHER; note = N1-methylpseudouracil
  modified_base 1129: mod_base = OTHER; note = N1-methylpseudouracil
  modified_base 1143: mod_base = OTHER; note = N1-methylpseudouracil
  modified_base 1150: mod_base = OTHER; note = N1-methylpseudouracil
  modified_base 1157: mod_base = OTHER; note = N1-methylpseudouracil
  modified_base 1160: mod_base = OTHER; note = N1-methylpseudouracil
  modified_base 1163: mod_base = OTHER; note = N1-methylpseudouracil
  modified_base 1166: mod_base = OTHER; note = N1-methylpseudouracil
  modified_base 1169: mod_base = OTHER; note = N1-methylpseudouracil
  modified_base 1175: mod_base = OTHER; note = N1-methylpseudouracil
  modified_base 1179: mod_base = OTHER; note = N1-methylpseudouracil
  modified_base 1186: mod_base = OTHER; note = N1-methylpseudouracil
  modified_base 1194: mod_base = OTHER; note = N1-methylpseudouracil
  modified_base 1199: mod_base = OTHER; note = N1-methylpseudouracil
  modified_base 1207: mod_base = OTHER; note = N1-methylpseudouracil
  modified_base 1210: mod_base = OTHER; note = N1-methylpseudouracil
  modified_base 1227: mod_base = OTHER; note = N1-methylpseudouracil
  modified_base 1234: mod_base = OTHER; note = N1-methylpseudouracil
  modified_base 1236: mod_base = OTHER; note = N1-methylpseudouracil
  modified_base 1237: mod_base = OTHER; note = N1-methylpseudouracil
  modified_base 1247: mod_base = OTHER; note = N1-methylpseudouracil
  modified_base 1249: mod_base = OTHER; note = N1-methylpseudouracil
  modified_base 1250: mod_base = OTHER; note = N1-methylpseudouracil
  modified_base 1258: mod_base = OTHER; note = N1-methylpseudouracil
  modified_base 1259: mod_base = OTHER; note = N1-methylpseudouracil
  modified_base 1266: mod_base = OTHER; note = N1-methylpseudouracil
  modified_base 1268: mod_base = OTHER; note = N1-methylpseudouracil
  modified_base 1271: mod_base = OTHER; note = N1-methylpseudouracil
  modified_base 1281: mod_base = OTHER; note = N1-methylpseudouracil
  modified_base 1289: mod_base = OTHER; note = N1-methylpseudouracil
  modified_base 1292: mod_base = OTHER; note = N1-methylpseudouracil
  modified_base 1303: mod_base = OTHER; note = N1-methylpseudouracil
  modified_base 1305: mod_base = OTHER; note = N1-methylpseudouracil
  modified_base 1310: mod_base = OTHER; note = N1-methylpseudouracil
  modified_base 1312: mod_base = OTHER; note = N1-methylpseudouracil
  modified_base 1313: mod_base = OTHER; note = N1-methylpseudouracil
  modified_base 1322: mod_base = OTHER; note = N1-methylpseudouracil
  modified_base 1330: mod_base = OTHER; note = N1-methylpseudouracil
  modified_base 1336: mod_base = OTHER; note = N1-methylpseudouracil
  modified_base 1337: mod_base = OTHER; note = N1-methylpseudouracil
  modified_base 1349: mod_base = OTHER; note = N1-methylpseudouracil
  modified_base 1351: mod_base = OTHER; note = N1-methylpseudouracil
  modified_base 1352: mod_base = OTHER; note = N1-methylpseudouracil
  modified_base 1355: mod_base = OTHER; note = N1-methylpseudouracil
  modified_base 1360: mod_base = OTHER; note = N1-methylpseudouracil
  modified_base 1373: mod_base = OTHER; note = N1-methylpseudouracil
  modified_base 1377: mod_base = OTHER; note = N1-methylpseudouracil
  modified_base 1382: mod_base = OTHER; note = N1-methylpseudouracil
  modified_base 1386: mod_base = OTHER; note = N1-methylpseudouracil
  modified_base 1392: mod_base = OTHER; note = N1-methylpseudouracil
  modified_base 1397: mod_base = OTHER; note = N1-methylpseudouracil
  modified_base 1400: mod_base = OTHER; note = N1-methylpseudouracil
  modified_base 1406: mod_base = OTHER; note = N1-methylpseudouracil
  modified_base 1407: mod_base = OTHER; note = N1-methylpseudouracil
  modified_base 1410: mod_base = OTHER; note = N1-methylpseudouracil
  modified_base 1418: mod_base = OTHER; note = N1-methylpseudouracil
  modified_base 1427: mod_base = OTHER; note = N1-methylpseudouracil
  modified_base 1438: mod_base = OTHER; note = N1-methylpseudouracil
  modified_base 1441: mod_base = OTHER; note = N1-methylpseudouracil
  modified_base 1443: mod_base = OTHER; note = N1-methylpseudouracil
  modified_base 1458: mod_base = OTHER; note = N1-methylpseudouracil
  modified_base 1466: mod_base = OTHER; note = N1-methylpseudouracil
  modified_base 1470: mod_base = OTHER; note = N1-methylpseudouracil
  modified_base 1471: mod_base = OTHER; note = N1-methylpseudouracil
  modified_base 1483: mod_base = OTHER; note = N1-methylpseudouracil
  modified_base 1487: mod_base = OTHER; note = N1-methylpseudouracil
  modified_base 1489: mod_base = OTHER; note = N1-methylpseudouracil
  modified_base 1493: mod_base = OTHER; note = N1-methylpseudouracil
  modified_base 1494: mod_base = OTHER; note = N1-methylpseudouracil
  modified_base 1496: mod_base = OTHER; note = N1-methylpseudouracil
  modified_base 1499: mod_base = OTHER; note = N1-methylpseudouracil
  modified_base 1508: mod_base = OTHER; note = N1-methylpseudouracil
  modified_base 1510: mod_base = OTHER; note = N1-methylpseudouracil
  modified_base 1514: mod_base = OTHER; note = N1-methylpseudouracil
  modified_base 1517: mod_base = OTHER; note = N1-methylpseudouracil
  modified_base 1533: mod_base = OTHER; note = N1-methylpseudouracil
  modified_base 1535: mod_base = OTHER; note = N1-methylpseudouracil
  modified_base 1547: mod_base = OTHER; note = N1-methylpseudouracil
  modified_base 1555: mod_base = OTHER; note = N1-methylpseudouracil
  modified_base 1557: mod_base = OTHER; note = N1-methylpseudouracil
  modified_base 1558: mod_base = OTHER; note = N1-methylpseudouracil
  modified_base 1570: mod_base = OTHER; note = N1-methylpseudouracil
  modified_base 1571: mod_base = OTHER; note = N1-methylpseudouracil
  modified_base 1574: mod_base = OTHER; note = N1-methylpseudouracil
  modified_base 1578: mod_base = OTHER; note = N1-methylpseudouracil
  modified_base 1587: mod_base = OTHER; note = N1-methylpseudouracil
  modified_base 1588: mod_base = OTHER; note = N1-methylpseudouracil
  modified_base 1591: mod_base = OTHER; note = N1-methylpseudouracil
  modified_base 1592: mod_base = OTHER; note = N1-methylpseudouracil
  modified_base 1601: mod_base = OTHER; note = N1-methylpseudouracil
  modified_base 1607: mod_base = OTHER; note = N1-methylpseudouracil
  modified_base 1608: mod_base = OTHER; note = N1-methylpseudouracil
  modified_base 1617: mod_base = OTHER; note = N1-methylpseudouracil
  modified_base 1618: mod_base = OTHER; note = N1-methylpseudouracil
  modified_base 1622: mod_base = OTHER; note = N1-methylpseudouracil
  modified_base 1626: mod_base = OTHER; note = N1-methylpseudouracil
  modified_base 1633: mod_base = OTHER; note = N1-methylpseudouracil
  modified_base 1634: mod_base = OTHER; note = N1-methylpseudouracil
  modified_base 1647: mod_base = OTHER; note = N1-methylpseudouracil
  modified_base 1648: mod_base = OTHER; note = N1-methylpseudouracil
  modified_base 1649: mod_base = OTHER; note = N1-methylpseudouracil
  modified_base 1654: mod_base = OTHER; note = N1-methylpseudouracil
  modified_base 1655: mod_base = OTHER; note = N1-methylpseudouracil
  modified_base 1667: mod_base = OTHER; note = N1-methylpseudouracil
  modified_base 1668: mod_base = OTHER; note = N1-methylpseudouracil
  modified_base 1669: mod_base = OTHER; note = N1-methylpseudouracil
  modified_base 1674: mod_base = OTHER; note = N1-methylpseudouracil
  modified_base 1676: mod_base = OTHER; note = N1-methylpseudouracil
  modified_base 1697: mod_base = OTHER; note = N1-methylpseudouracil
  modified_base 1715: mod_base = OTHER; note = N1-methylpseudouracil
  modified_base 1718: mod_base = OTHER; note = N1-methylpseudouracil
  modified_base 1723: mod_base = OTHER; note = N1-methylpseudouracil
  modified_base 1724: mod_base = OTHER; note = N1-methylpseudouracil
  modified_base 1727: mod_base = OTHER; note = N1-methylpseudouracil
  modified_base 1760: mod_base = OTHER; note = N1-methylpseudouracil
  modified_base 1761: mod_base = OTHER; note = N1-methylpseudouracil
  modified_base 1769: mod_base = OTHER; note = N1-methylpseudouracil
  modified_base 1772: mod_base = OTHER; note = N1-methylpseudouracil
  modified_base 1780: mod_base = OTHER; note = N1-methylpseudouracil
  modified_base 1781: mod_base = OTHER; note = N1-methylpseudouracil
  modified_base 1789: mod_base = OTHER; note = N1-methylpseudouracil
  modified_base 1790: mod_base = OTHER; note = N1-methylpseudouracil
  modified_base 1793: mod_base = OTHER; note = N1-methylpseudouracil
  modified_base 1801: mod_base = OTHER; note = N1-methylpseudouracil
  modified_base 1803: mod_base = OTHER; note = N1-methylpseudouracil
  modified_base 1804: mod_base = OTHER; note = N1-methylpseudouracil
  modified_base 1806: mod_base = OTHER; note = N1-methylpseudouracil
  modified_base 1827: mod_base = OTHER; note = N1-methylpseudouracil
  modified_base 1828: mod_base = OTHER; note = N1-methylpseudouracil
  modified_base 1831: mod_base = OTHER; note = N1-methylpseudouracil
  modified_base 1832: mod_base = OTHER; note = N1-methylpseudouracil
  modified_base 1852: mod_base = OTHER; note = N1-methylpseudouracil
  modified_base 1853: mod_base = OTHER; note = N1-methylpseudouracil
  modified_base 1856: mod_base = OTHER; note = N1-methylpseudouracil
  modified_base 1864: mod_base = OTHER; note = N1-methylpseudouracil
  modified_base 1866: mod_base = OTHER; note = N1-methylpseudouracil
  modified_base 1872: mod_base = OTHER; note = N1-methylpseudouracil
  modified_base 1876: mod_base = OTHER; note = N1-methylpseudouracil
  modified_base 1877: mod_base = OTHER; note = N1-methylpseudouracil
  modified_base 1883: mod_base = OTHER; note = N1-methylpseudouracil
  modified_base 1887: mod_base = OTHER; note = N1-methylpseudouracil
  modified_base 1902: mod_base = OTHER; note = N1-methylpseudouracil
  modified_base 1912: mod_base = OTHER; note = N1-methylpseudouracil
  modified_base 1931: mod_base = OTHER; note = N1-methylpseudouracil
  modified_base 1937: mod_base = OTHER; note = N1-methylpseudouracil
  modified_base 1946: mod_base = OTHER; note = N1-methylpseudouracil
  modified_base 1949: mod_base = OTHER; note = N1-methylpseudouracil
  modified_base 1952: mod_base = OTHER; note = N1-methylpseudouracil
  modified_base 1957: mod_base = OTHER; note = N1-methylpseudouracil
  modified_base 1958: mod_base = OTHER; note = N1-methylpseudouracil
  modified_base 1964: mod_base = OTHER; note = N1-methylpseudouracil
  modified_base 1967: mod_base = OTHER; note = N1-methylpseudouracil
  modified_base 1970: mod_base = OTHER; note = N1-methylpseudouracil
  modified_base 1979: mod_base = OTHER; note = N1-methylpseudouracil
  modified_base 1987: mod_base = OTHER; note = N1-methylpseudouracil
  modified_base 2003: mod_base = OTHER; note = N1-methylpseudouracil
  modified_base 2012: mod_base = OTHER; note = N1-methylpseudouracil
  modified_base 2015: mod_base = OTHER; note = N1-methylpseudouracil
  modified_base 2020: mod_base = OTHER; note = N1-methylpseudouracil
  modified_base 2021: mod_base = OTHER; note = N1-methylpseudouracil
  modified_base 2026: mod_base = OTHER; note = N1-methylpseudouracil
  modified_base 2027: mod_base = OTHER; note = N1-methylpseudouracil
  modified_base 2029: mod_base = OTHER; note = N1-methylpseudouracil
  modified_base 2033: mod_base = OTHER; note = N1-methylpseudouracil
  modified_base 2037: mod_base = OTHER; note = N1-methylpseudouracil
  modified_base 2049: mod_base = OTHER; note = N1-methylpseudouracil
  modified_base 2054: mod_base = OTHER; note = N1-methylpseudouracil
  modified_base 2063: mod_base = OTHER; note = N1-methylpseudouracil
  modified_base 2064: mod_base = OTHER; note = N1-methylpseudouracil
  modified_base 2068: mod_base = OTHER; note = N1-methylpseudouracil
  modified_base 2072: mod_base = OTHER; note = N1-methylpseudouracil
  modified_base 2084: mod_base = OTHER; note = N1-methylpseudouracil
  modified_base 2085: mod_base = OTHER; note = N1-methylpseudouracil
  modified_base 2093: mod_base = OTHER; note = N1-methylpseudouracil
  modified_base 2105: mod_base = OTHER; note = N1-methylpseudouracil
  modified_base 2111: mod_base = OTHER; note = N1-methylpseudouracil
  modified_base 2118: mod_base = OTHER; note = N1-methylpseudouracil
  modified_base 2121: mod_base = OTHER; note = N1-methylpseudouracil
  modified_base 2127: mod_base = OTHER; note = N1-methylpseudouracil
  modified_base 2128: mod_base = OTHER; note = N1-methylpseudouracil
  modified_base 2129: mod_base = OTHER; note = N1-methylpseudouracil
  modified_base 2135: mod_base = OTHER; note = N1-methylpseudouracil
  modified_base 2142: mod_base = OTHER; note = N1-methylpseudouracil
  modified_base 2150: mod_base = OTHER; note = N1-methylpseudouracil
  modified_base 2151: mod_base = OTHER; note = N1-methylpseudouracil
  modified_base 2153: mod_base = OTHER; note = N1-methylpseudouracil
  modified_base 2154: mod_base = OTHER; note = N1-methylpseudouracil
  modified_base 2162: mod_base = OTHER; note = N1-methylpseudouracil
  modified_base 2168: mod_base = OTHER; note = N1-methylpseudouracil
  modified_base 2171: mod_base = OTHER; note = N1-methylpseudouracil
  modified_base 2174: mod_base = OTHER; note = N1-methylpseudouracil
  modified_base 2177: mod_base = OTHER; note = N1-methylpseudouracil
  modified_base 2182: mod_base = OTHER; note = N1-methylpseudouracil
  modified_base 2183: mod_base = OTHER; note = N1-methylpseudouracil
  modified_base 2191: mod_base = OTHER; note = N1-methylpseudouracil
  modified_base 2203: mod_base = OTHER; note = N1-methylpseudouracil
  modified_base 2204: mod_base = OTHER; note = N1-methylpseudouracil
  modified_base 2207: mod_base = OTHER; note = N1-methylpseudouracil
  modified_base 2209: mod_base = OTHER; note = N1-methylpseudouracil
  modified_base 2215: mod_base = OTHER; note = N1-methylpseudouracil
  modified_base 2237: mod_base = OTHER; note = N1-methylpseudouracil
  modified_base 2240: mod_base = OTHER; note = N1-methylpseudouracil
  modified_base 2245: mod_base = OTHER; note = N1-methylpseudouracil
  modified_base 2246: mod_base = OTHER; note = N1-methylpseudouracil
  modified_base 2249: mod_base = OTHER; note = N1-methylpseudouracil
  modified_base 2257: mod_base = OTHER; note = N1-methylpseudouracil
  modified_base 2261: mod_base = OTHER; note = N1-methylpseudouracil
  modified_base 2266: mod_base = OTHER; note = N1-methylpseudouracil
  modified_base 2267: mod_base = OTHER; note = N1-methylpseudouracil
  modified_base 2269: mod_base = OTHER; note = N1-methylpseudouracil
  modified_base 2279: mod_base = OTHER; note = N1-methylpseudouracil
  modified_base 2282: mod_base = OTHER; note = N1-methylpseudouracil
  modified_base 2284: mod_base = OTHER; note = N1-methylpseudouracil
  modified_base 2286: mod_base = OTHER; note = N1-methylpseudouracil
  modified_base 2289: mod_base = OTHER; note = N1-methylpseudouracil
  modified_base 2291: mod_base = OTHER; note = N1-methylpseudouracil
  modified_base 2296: mod_base = OTHER; note = N1-methylpseudouracil
  modified_base 2299: mod_base = OTHER; note = N1-methylpseudouracil
  modified_base 2302: mod_base = OTHER; note = N1-methylpseudouracil
  modified_base 2307: mod_base = OTHER; note = N1-methylpseudouracil
  modified_base 2314: mod_base = OTHER; note = N1-methylpseudouracil
  modified_base 2318: mod_base = OTHER; note = N1-methylpseudouracil
  modified_base 2323: mod_base = OTHER; note = N1-methylpseudouracil
  modified_base 2327: mod_base = OTHER; note = N1-methylpseudouracil
  modified_base 2328: mod_base = OTHER; note = N1-methylpseudouracil
  modified_base 2330: mod_base = OTHER; note = N1-methylpseudouracil
  modified_base 2331: mod_base = OTHER; note = N1-methylpseudouracil
  modified_base 2337: mod_base = OTHER; note = N1-methylpseudouracil
  modified_base 2338: mod_base = OTHER; note = N1-methylpseudouracil
  modified_base 2344: mod_base = OTHER; note = N1-methylpseudouracil
  modified_base 2357: mod_base = OTHER; note = N1-methylpseudouracil
  modified_base 2360: mod_base = OTHER; note = N1-methylpseudouracil
  modified_base 2365: mod_base = OTHER; note = N1-methylpseudouracil
  modified_base 2366: mod_base = OTHER; note = N1-methylpseudouracil
  modified_base 2369: mod_base = OTHER; note = N1-methylpseudouracil
  modified_base 2377: mod_base = OTHER; note = N1-methylpseudouracil
  modified_base 2385: mod_base = OTHER; note = N1-methylpseudouracil
  modified_base 2388: mod_base = OTHER; note = N1-methylpseudouracil
  modified_base 2390: mod_base = OTHER; note = N1-methylpseudouracil
  modified_base 2391: mod_base = OTHER; note = N1-methylpseudouracil
  modified_base 2392: mod_base = OTHER; note = N1-methylpseudouracil
  modified_base 2396: mod_base = OTHER; note = N1-methylpseudouracil
  modified_base 2401: mod_base = OTHER; note = N1-methylpseudouracil
  modified_base 2403: mod_base = OTHER; note = N1-methylpseudouracil
  modified_base 2407: mod_base = OTHER; note = N1-methylpseudouracil
gggaaanaagagagaaaagaagagnaagaagaaananaagaccccggcgccgccaccangaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacannngcacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngaccnganaanaggcnggagccncggnggccnagcnncnngccccnngggccnccccccagccccnccnccccnnccngcacccgnacccccgnggncnnngaanaaagncngagngggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - protein, 9 aa, synthetic construct
  VARIANT 1: note = G, H, or absent
  VARIANT 2: note = D, G, or absent
  VARIANT 3: note = D, V, I, S, or M
  VARIANT 5: note = Any amino acid
XXXEXNPGP

SEQ ID NO: 39 - protein, 8 aa, synthetic construct
  VARIANT 2: note = V or I
  VARIANT 4: note = Any amino acid
DXEXNPGP

SEQ ID NO: 40 - protein, 9 aa, synthetic construct
GDVESNPGP

SEQ ID NO: 41 - protein, 9 aa, synthetic construct
GDIEENPGP

SEQ ID NO: 42 - protein, 7 aa, synthetic construct
VEPNPGP

SEQ ID NO: 43 - protein, 7 aa, synthetic construct
IETNPGP

SEQ ID NO: 44 - protein, 9 aa, synthetic construct
GDIESNPGP

SEQ ID NO: 45 - protein, 9 aa, synthetic construct
GDVELNPGP

SEQ ID NO: 46 - protein, 9 aa, synthetic construct
GDIETNPGP

SEQ ID NO: 47 - protein, 8 aa, synthetic construct
GDVENPGP

SEQ ID NO: 48 - protein, 9 aa, synthetic construct
GDVEENPGP

SEQ ID NO: 49 - protein, 9 aa, synthetic construct
GDVEQNPGP

SEQ ID NO: 50 - protein, 7 aa, synthetic construct
IESNPGP

SEQ ID NO: 51 - protein, 9 aa, synthetic construct
GDIELNPGP

SEQ ID NO: 52 - protein, 9 aa, synthetic construct
HDIETNPGP

SEQ ID NO: 53 - protein, 9 aa, synthetic construct
HDVETNPGP

SEQ ID NO: 54 - protein, 9 aa, synthetic construct
HDVEMNPGP

SEQ ID NO: 55 - protein, 9 aa, synthetic construct
GDMESNPGP

SEQ ID NO: 56 - protein, 9 aa, synthetic construct
GDVETNPGP

SEQ ID NO: 57 - protein, 9 aa, synthetic construct
GDIEQNPGP

SEQ ID NO: 58 - protein, 8 aa, synthetic construct
DSEFNPGP

SEQ ID NO: 59 - DNA, 1431 bp, synthetic construct
  source 1..1431: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 60 - DNA, 1491 bp, synthetic construct
  source 1..1491: mol_type = other DNA
atgtggtggcgcctgtggtggctgctgctgctgctgctgctgctgtggcccatggtgtgggccgtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 61 - DNA, 1473 bp, synthetic construct
  source 1..1473: mol_type = other DNA
atgggctgcgtgtgcagcagcaaccccgagaacaacaacaacatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 62 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 63 - DNA, 462 bp, synthetic construct
  source 1..462: mol_type = other DNA
atgtacaggatgcaactcctgtcttgcattgcactaagtcttgcacttgtcacaaacagtgcacccaccagcagcagcaccaagaagacccagctgcagctggagcacctgctgctggacctgcagatgatcctgaacggcatcaacaactacaagaaccccaagctgaccagaatgctgaccttcaagttctacatgcccaagaaggccaccgagctgaagcacctgcagtgcctggaggaggagctgaagcccctggaggaggtgctgaacctggcccagagcaagaacttccacctgagacccagagacctgatcagcaacatcaacgtgatcgtgctggagctgaagggcagcgagaccaccttcatgtgcgagtacgccgacgagaccgccaccatcgtggagttcctgaacagatggatcaccttcagccagagcatcatcagcaccctgacctga

SEQ ID NO: 64 - DNA, 462 bp, synthetic construct
  source 1..462: mol_type = other DNA
atgtacaggatgcaactcctgtcttgcattgcactaagtcttgcacttgtcacaaacagtgcacccaccagcagcagcaccaagaagacccagctgcagctggagcacctgctgctggacctgcagatgatcctgaacggcatcaacaactacaagaaccccaagctgaccagaatgctgaccgccaagttctacatgcccaagaaggccaccgagctgaagcacctgcagtgcctggaggaggagctgaagcccctggaggaggtgctgaacctggcccagagcaagaacttccacttcgaccccagagacgtggtgagcaacatcaacgtgttcgtgctggagctgaagggcagcgagaccaccttcatgtgcgagtacgccgacgagaccgccaccatcgtggagttcctgaacagatggatcaccttcagccagagcatcatcagcaccctgacctga

SEQ ID NO: 65 - DNA, 972 bp, synthetic construct
  source 1..972: mol_type = other DNA
atgtacaggatgcaactcctgtcttgcattgcactaagtcttgcacttgtcacaaacagtagcaagaacttccacctgagacccagagacctgatcagcaacatcaacgtgatcgtgctggagctgaagggcagcgagaccaccttcatgtgcgagtacgccgacgagaccgccaccatcgtggagttcctgaacagatggatcaccttcagccagagcatcatcagcaccctgaccggcggcagcagcagcaccaagaagacccagctgcagctggagcacctgctgctggacctgcagatgatcctgaacggcatcaacaactacaagaaccccaagctgaccagaatgctgaccttcaagttctacatgcccaagaaggccaccgagctgaagcacctgcagtgcctggaggaggagctgaagcccctggaggaggtgctgaacctggcccagggcagcggcggcggcagcgagctgtgcgacgacgacccccccgagatcccccacgccaccttcaaggccatggcctacaaggagggcaccatgctgaactgcgagtgcaagagaggcttcagaagaatcaagagcggcagcctgtacatgctgtgcaccggcaacagcagccacagcagctgggacaaccagtgccagtgcaccagcagcgccaccagaaacaccaccaagcaggtgaccccccagcccgaggagcagaaggagagaaagaccaccgagatgcagagccccatgcagcccgtggaccaggccagcctgcccggccactgcagagagccccccccctgggagaacgaggccaccgagagaatctaccacttcgtggtgggccagatggtgtactaccagtgcgtgcagggctacagagccctgcacagaggccccgccgagagcgtgtgcaagatgacccacggcaagaccagatggacccagccccagctgatctgcaccggctga

SEQ ID NO: 66 - DNA, 2286 bp, synthetic construct
  source 1..2286: mol_type = other DNA
atgctgctgctggtgaccagcctgctgctgtgcgagctgccccaccccgccttccttttgattccagaggtgcaattacagcaaagcggtgcagaactcgtgaagccaggcgcttcagtaaaaatgtcctgcaaagcttccggctacacattcactagctacaacatgcattgggttaaacaaacacctggccagggattggagtggatcggggctatatatccgggtaacggtgacactagttacaatcagaaattcaagggaaaggccacgcttacagcagacaagagtagcagtaccgcctatatgcaactcagcagcttgaccagcgaagacagcgcagattattactgtgccagatccaactattacggctcaagctactggttctttgacgtgtggggcgccggaaccactgttactgtcagcagcggaggcggtgggagtggcggcggtggttccggcgggggcgggtcagatattgtgttgacgcaatcccccgcaatcctgtccgcgtccccaggcgaaaaagtgacaatgacatgccgggccagctcaagcgtgaattacatggattggtatcagaagaaacccgggagcagccccaagccgtggatctatgcgacgagtaacctggcttccggcgtccccgcccgattcagcgggtccggcagcggcactagttacagtttaaccatcagcagggtggaggctgaagacgcggcaacctattattgccagcagtggtcatttaatcctcccacgtttggcggcggcaccaagctggagataaagggcggggggggcagtggaggcggaggtagcggcggaggaggatcaggaggggggggtagcggaggagggggttcagacatacagatgacacagacaacctcgagcctgagcgcatccctgggggaccgagtcaccatctcatgtagagcatcgcaggacatcagcaaatatctcaactggtaccagcaaaaacccgatggtacagtgaaactcttaatttaccacacatcccggttacatagcggagtcccaagccgctttagcggatccggaagcgggaccgactactccctcacgatcagcaacctcgaacaggaggatatagccacttatttctgccagcagggtaacaccctgccgtatacatttggcgggggcacaaaactagaaatcacaggcagtacaagtggtagtgggaagcccggtagcggagaaggtagcacgaaaggcgaggttaaacttcaggaatcaggcccaggcctagttgccccttcacaaagtctaagcgtcacgtgtaccgtgtccggcgtaagcctgccagattatggagtcagctggattaggcagcctccacggaagggactggagtggctgggcgtgatttgggggtcggagaccacctattataacagcgcgctgaaatcgcggctgactattattaaggataatagcaagtcacaggtgtttcttaagatgaatagcctgcagactgatgacactgcgatatactactgtgccaagcactactactacggagggtcatatgctatggattactgggggcagggaacctccgtgactgtaagttccgccgccgctactacaacccctgccccacgtcccccgaccccagcacctactatcgcatcccagcccctttcccttcgccctgaggcctgcaggcctgcagctggaggagccgttcacaccaggggcctcgactttgcatgcgatatttacatctgggccccactggctggcacctgcggcgtcttgctgctgagcctcgtgatcaccctctattgtaagagggggcgaaaaaagctcctgtacattttcaaacagcctttcatgagacctgtgcagacaacacaggaagaggacggttgctcctgtaggtttccggaggaagaagagggagggtgtgaattgcgtgtaaagttctccagatcagctgatgccccagcctatcaacaaggacagaatcagctgtacaacgagcttaatttggggcggagagaggaatacgatgtcttggacaagagacgggggcgcgaccccgagatgggcgggaagcctagaaggaagaaccctcaggaggggctgtataatgagctccaaaaggataagatggccgaagcttacagcgagatcgggatgaaaggagagcgccgccgcggaaaaggccatgatgggttataccagggccttagcaccgccactaaggacacttacgacgctctacacatgcaggctctcccaccccgatga

SEQ ID NO: 67 - DNA, 1404 bp, synthetic construct
  source 1..1404: mol_type = other DNA
atgagggcttggatcttctttctgctctgcctggccgggcgcgccttggccgaagttcaactggtagagagtggaggtgggcttgtgcaaccaggcggatccttgcgactgtcctgcgccgcttcaggcttcaacatcaaggacacctacatccattgggtccgccaggcaccaggaaaaggtcttgaatgggtggccagaatctaccctactaacggttacaccagatatgcagactccgttaaggggcgatttaccatttcagcagacacctctaagaacaccgcttacctgcagatgaactcacttcgagctgaggacaccgccgtttactattgcagcagatggggcggtgacggcttctacgctatggattactggggacaggggactttggtaactgtgagtagtgcatctacaaaggggccaagcgtgttcccacttgccccatcttctaaaagcacctcaggagggactgcagccttgggttgcttggttaaagattattttccagagcctgtaactgtatcctggaatagtggggccctcacaagcggagtacatactttccctgcagtattgcagtctagtggactctactctctcagcagtgtagtgaccgtaccttccagttcacttggaacacagacctatatttgcaatgtgaatcataagccatctaatactaaagtggataagaaagtggagcctaaatcttgtgacaagactcatacatgccctccctgccctgcccctgaactgttgggagggccctctgtatttctcttcccccctaaaccaaaggacaccctgatgatcagtcgaactcctgaggtgacttgtgtggttgttgacgtgtcacatgaggatcccgaagtgaaattcaactggtacgtcgatggagtagaggtacacaatgcaaagacaaaacctagggaggaacagtataattctacctatagagtggtgtctgttctcacagttctccatcaagactggttgaacggtaaagaatataaatgcaaagtctccaataaggctttgcccgctcccattgaaaaaacaatcagtaaagccaaaggccagccacgcgaaccacaggtctacacccttccaccatctcgcgaagaaatgactaagaatcaagtgtcactcacatgcctcgtcaagggcttttacccttctgatattgcagttgaatgggagtctaacgggcagcccgagaataattacaagactaccccccccgtccttgactctgatggcagctttttcctgtactcaaaattgactgtggacaagtctcgatggcaacagggtaatgttttctcctgtagcgtaatgcacgaggcccttcataaccattatacccaaaaatctctttcattgtcccctggaaaatga

SEQ ID NO: 68 - DNA, 696 bp, synthetic construct
  source 1..696: mol_type = other DNA
atgagggcttggatcttctttctgctctgcctggccgggcgcgccttggccgatattcagatgactcagagccccagcagcctgtctgctagcgttggggatagagtcactataacatgtcgggcttcccaggatgtgaatactgctgtcgcttggtatcaacagaagcccggcaaggcaccaaaactgctgatatatagtgcctctttcctttactccggggttcccagtcgattctctggaagccgcagcggcactgatttcacacttactataagtagtctgcaacctgaggactttgctacatactactgccagcagcactatacaacacccccaactttcgggcaaggcactaaggtagaaattaaaaggaccgttgctgctccatccgtctttatttttccaccatctgatgaacagttgaagagtggaacagcaagcgtcgtttgtctcctgaacaatttttacccacgagaggcaaaagttcaatggaaggtagacaatgcccttcagagcggcaattcccaggagagcgtaaccgagcaggatagcaaagactctacatactctttgagttcaacccttaccctgagcaaggctgattacgaaaaacacaaggtgtacgcttgcgaggtaacccatcagggattgtcatccccagtcacaaaatcattcaataggggcgagtgctga

SEQ ID NO: 69 - RNA, 1398 nt, synthetic construct
  source 1..1398: mol_type = other RNA
atggaatttggactgtcttgggtttttctggtagctctgtttcgtggagtgcaatgccaggttcagttggtccagtcgggtgtggaggttaaaaagcccggcgcctctgtgaaggttagctgcaaggctagcggatatactttcaccaattactacatgtattgggtgaggcaggctccagggcagggactcgagtggatgggaggcatcaacccgtccaacggcggcaccaacttcaatgagaagttcaaaaatagagtgactctcaccactgatagttccacgacaaccgcatacatggaactgaaaagtctgcaattcgatgacaccgccgtgtattactgcgcccgcagagactaccggtttgacatgggatttgattactggggtcagggcaccacagtgacagtctcatccgcctctaccaaaggaccctcggtttttccactcgcaccttgctcccgaagcacctcagagtccaccgcggcccttgggtgtctggtgaaagattatttccccgaaccagtgaccgtgtcatggaattcaggggcactgacaagtggggtccacacgttcccagcggtgctgcagtcctccgggctgtatagccttagctctgtagtcactgtcccgtccagttccctggggactaagacttacacatgtaacgtagatcacaaacctagtaacaccaaggtggacaagcgcgtggagtcaaagtacggacctccatgtcccccatgtcccgcccccgaatttttgggaggaccttcagtgttcctgttccctccaaaacctaaagacacactgatgataagcaggacacccgaggtcacctgcgtcgtggtcgacgtaagccaggaagatccagaggtacagttcaactggtacgtcgacggtgtagaggtccataatgccaagacaaagcccagggaggagcagtttaactcaacatatcgtgttgtgtctgtgttgacggttctacatcaagactggctaaacggcaaggagtataaatgcaaggttagtaataagggcctccccagctctattgagaaaactatcagcaaagctaagggccagcctcgggaaccacaagtttacactttaccgcccagccaggaggaaatgacgaagaatcaggtgagcctgacatgtcttgtcaaggggttttacccctctgatatcgctgtagaatgggaatccaatggtcaacctgaaaacaactacaagaccaccccgcctgtgctcgactctgacggcagctttttcctgtattcgagacttacagtggataaaagccggtggcaggagggcaatgtgttttcctgcagtgtgatgcacgaagcattacacaaccattatactcaaaaatcactcagtttgtctctggggaag

SEQ ID NO: 70 - RNA, 579 nt, synthetic construct
  source 1..579: mol_type = other RNA
atgggggtccacgaatgcccggcatggctgtggttactgctatcgctgctcagcctgccactgggcctgcccgtacttggcgcccctccacgcctcatctgtgactcccgggtcctggagcggtaccttttggaggccaaagaagcagagaatatcactacaggctgcgctgagcactgtagtttgaacgaaaatataacagtacctgacacaaaggtgaatttttacgcctggaaaagaatggaggtgggccaacaggcagtggaagtgtggcagggtctcgctttacttagcgaggccgtgctgcgaggtcaggcactcctagttaactccagccagccctgggagccactccagttgcatgtggataaggccgtctctggactaagatccctcaccaccctgctgagggcattgggagcgcaaaaggaggctatttcacctcccgatgccgctagtgctgcgccccttcgcactatcaccgccgacacgttccgtaagctgttcagggtttattcaaactttctccgggggaagctgaaactgtatactggggaagcctgcagaaccggagatagg

SEQ ID NO: 71 - RNA, 1455 nt, synthetic construct
  source 1..1455: mol_type = other RNA
atgaaatgggttacgttcatctccttgctgttcctgttctcttccgcttactccctgccagcccaggtcgctttcacgccatacgctcctgaaccaggtagcacttgtcgtctacgggaatactatgaccagactgcacagatgtgctgttccaagtgctctcctggccaacatgccaaagtgttttgcactaagactagcgatacagtgtgcgatagctgcgaggattctacatatacccagctctggaactgggttcccgagtgtctcagctgtggcagccggtgtagtagcgaccaggtggagacccaggcctgcacacgagagcagaaccggatctgcacatgtagacccgggtggtactgtgcgttgagtaaacaggagggatgtagactctgcgcccctcttaggaagtgccgcccagggtttggagtcgcacgcccaggcacagaaaccagtgacgtcgtctgcaagccgtgtgccccgggcacattctccaacactacctcaagtacagatatttgtagacctcatcagatctgcaatgtcgtggcaattcccggtaacgcctcgatggatgccgtgtgcacctctacgtcgcccactaggtccatggccccaggagcagtacacttacctcagcctgttagcacacgctcccagcacacgcaacccacccctgagccttccactgcaccgtccaccagcttcctgctgcccatgggcccaagtcctccagcggagggcagcacaggggacgagccaaagtcctgcgacaaaacccatacttgtcccccttgccccgcacccgaactccttgggggaccatcagtattcttgtttccccccaagccaaaggacacattaatgatttctaggactccggaggtgacttgcgtcgttgttgacgtgagccacgaagacccagaggtgaaattcaactggtatgtggatggagtcgaagttcacaatgcgaaaaccaaacctcgagaggaacagtacaactcaacctatagggtagtgagtgttctgaccgtgctgcaccaggactggctcaatgggaaggaatacaagtgtaaggtatccaataaagccctgcctgcccctatcgaaaagacaataagcaaggctaaggggcaacctcgcgaaccccaggtgtacacgctgccaccaagcagagaggagatgacaaaaaaccaagtgtcactgacctgcctggtgaaaggcttttacccgagcgatatcgctgtggagtgggagtcaaacggacaacccgagaataactataaaaccacgccccccgtgctggattcagacggttctttcttcctttattccaagttgaccgtggacaagtctcggtggcagcagggcaatgtcttttcctgtagtgtcatgcacgaagctctacacaatcattacacccaaaaaagcctgtcactctctcccggtaaa

SEQ ID NO: 72 - RNA, 612 nt, synthetic construct
  source 1..612: mol_type = other RNA
atggcaggcccagctacgcagagcccaatgaaactgatggcactgcagctcctgttatggcacagcgccctgtggaccgtccaggaagcaacacccctcgggcctgcgtccagtctcccccagagctttctacttaaatgtctagagcaggtgcggaagatccaaggagacggggccgcactgcaggagaagctctgcgctacatacaagctttgtcacccagaggaactggtgctgttaggccattcactgggaatcccttgggcccctttgtctagttgcccgtcccaggctttacagttggccggctgcctgagccagctgcactccggactatttctgtatcagggacttttgcaggccctggagggtatttcccctgagctcgggccgactctggatacactgcagcttgatgtcgccgactttgctactaccatatggcaacaaatggaggaactcggtatggccccagcgctgcaacccacccagggggccatgcccgctttcgcgtctgccttccagcgcagagcaggcggcgtgttggtggcctcacatctccaatcgttcctggaagtttcttaccgagtactcaggcacttggcacagccc

SEQ ID NO: 73 - RNA, 1410 nt, synthetic construct
  source 1..1410: mol_type = other RNA
atggaatttggactgtcttgggtttttctggtagctctgtttcgtggagtgcaatgcgaggttcagcttgtcgagagcggcggcggtctcgtgcaacccggacggagtctgagactcagctgtgctgcttcaggatttaccttcgacgattatgcgatgcattgggtgcggcaggctcctggcaaaggattagaatgggtgagcgcaattacatggaactctggccacatcgactacgccgattcagtggagggcaggttcaccattagcagagataatgctaagaatagcttatacttgcagatgaacagcctgcgcgccgaggatactgcggtgtactattgcgccaaggtcagttatctgtccaccgccagctccctagattattgggggcagggaaccttggtgacagtcagttccgcaagcacgaaagggcccagcgttttccctctcgcaccctcttcaaaatccacttcaggggggacagctgcactgggatgtcttgtcaaggattacttccccgaacctgtgaccgtctcatggaattccggtgccttgacctcaggggtgcataccttccccgcagtccttcagagttctggactctactccctgagttctgtggtaacagtcccttctagctccctcgggacacagacctacatttgcaatgtgaaccataaaccgagtaacactaaagtggacaaaaaagtagaacccaaaagctgcgataagacccacacgtgccccccgtgtccggcccccgagcttctgggcggcccttcggtgttcctgtttccacctaagcccaaagacacactgatgatctctcgcactcctgaagtaacatgcgtggtggtcgacgtgagccacgaagatccagaagttaagttcaattggtatgttgacggggtggaggtgcacaacgccaagactaagccaagggaggagcaatacaattccacataccgagttgtcagcgtacttacggttctgcaccaagactggctaaatggtaaagagtacaagtgtaaggtgtctaacaaagccctgccagcgccgatagaaaagactatctcaaaagcaaagggccagcccagagaacctcaggtgtacaccttgccaccatcaagggacgagctcaccaagaatcaggttagtctcacctgtctggtgaaggggttctatccatccgacatcgccgtggagtgggagagtaacggccagcccgaaaacaattacaagacgacaccaccagtcttggatagcgacggttctttctttctatattcgaagctcactgtcgacaagtcgcggtggcagcagggcaacgtatttagctgctccgtcatgcatgaggccctgcacaaccactatactcaaaaatccctgtccttatctcctggcaag

SEQ ID NO: 74 - RNA, 705 nt, synthetic construct
  source 1..705: mol_type = other RNA
atgaagtatctactgcctaccgctgcagctgggctgttgcttctggctgctcaaccagccatggctcagatcgtgctctcccagtcccccgcgattttgagcgcatccccaggcgagaaggtaacaatgacgtgccgcgcgagcagttcagtgtcttacatccactggttccagcaaaagccaggctctagccccaagccctggatttacgcaaccagcaatcttgccagtggcgtcccggtgaggttctcggggtctggaagcggtacatcttatagtctcaccataagcagggtagaggccgaggacgccgctacttactattgccagcagtggacctctaacccacccaccttcggcggtggaaccaagctggaaatcaaacgaacagtcgccgccccgtccgtgtttatcttccctccttctgacgagcagctgaaatccgggaccgcatccgtcgtttgtctactgaacaacttttacccccgggaagcaaaagtccagtggaaggttgataacgccctccaaagtggaaatagccaggaatcagtgactgagcaagattccaaagatagtacttacagcttatcaagcacgctcactctgtccaaagccgactatgaaaaacacaaggtgtacgcctgtgaggtgacacatcaggggctgtcatcacctgttacaaagtcgtttaatagaggcgaatgc

SEQ ID NO: 75 - RNA, 858 nt, synthetic construct
  source 1..858: mol_type = other RNA
atgaaatgggttacgttcatctccttgctgttcctgttctcttccgcttactccgataaaacacatacctgtcccccatgtcccgcccccgagctcctcggcggtcccagtgtgtttctgtttccaccaaaacccaaggacactctcatgataagccgcacgcccgaggttacatgcgtcgtcgtcgacgtctcccacgaggatccggaggtgaaatttaactggtatgtagatggcgtggaagtgcacaacgcaaaaaccaagccccgtgaagaacaatacaactccacatacagggtcgtgagcgtgctgactgtactccatcaggattggttaaatggcaaggagtataagtgtaaggtgagtaataaggccctacctgcacctattgaaaagacaattagcaaagccaagggccagccgagggagccacaggtgtatacactgccacccagccgggatgagttaacaaagaatcaggtttcactcacctgccttgtaaaaggtttttacccatcagacatcgctgtggaatgggagtctaacggacagcctgaaaacaactataaaaccactcctcctgtgctggactctgacggatcgttcttcttgtactcaaaactaaccgtggacaagagtagatggcagcagggaaatgttttctcatgcagcgtcatgcacgaggccctgcataatcactacacccagaagagtttgtctctgagcccgggtaagggaggcggcgggggcatcgaagggcctactctgagacaatggctggcagcccgggctggcgggggtggaggggggggaggcatcgaggggccaacccttcgccaatggcttgcggcgcgagcc

SEQ ID NO: 76 - RNA, 432 nt, synthetic construct
  source 1..432: mol_type = other RNA
atgtggctgcaatccctgctgctgcttggcacagtggcatgctccatcagcgctcctgcccgatctccctcacccagcacacaaccctgggagcatgtcaatgcgatacaggaggccctccggctcttaaatctctcgcgcgatactgcagctgagatgaacgaaaccgtggaagtgatcagtgaaatgttcgatctgcaggaacccacctgtctgcagaccaggctagagttgtacaaacagggcttgagaggatcactaactaaacttaaggggcctctgacgatgatggcctcccactataagcagcactgtccaccaactccagaaacaagttgcgccacacagatcattacctttgagagctttaaggagaacttaaaagacttcctcctggttattcctttcgactgctgggagccggtacaggag

SEQ ID NO: 77 - RNA, 1425 nt, synthetic construct
  source 1..1425: mol_type = other RNA
atgggtgtccctacccagctcctgggactgctcctgctgtggatcaccgacgccatctgcgacatccagatgacccagagccctgccagcctgtctaccagcctgggcgagacagtgaccatccagtgtcaggccagcgaggacatctactctggcctggcttggtatcagcagaagcccggcaagagccctcagctgctgatctacggcgccagcgacctgcaggacggcgtgcctagcagattcagcggcagcggctccggaacccagtacagcctgaagatcaccagcatgcagaccgaggacgagggcgtgtacttctgccagcaaggcctgacctaccctagaaccttcggaggaggcaccaagctggaactgaagggcggaggcggaagtggaggcggaggatctggcggcggaggctctgaagtgcagctgcagcagtctggcgctgaactggtccggcctggcactagcgtgaagctgtcctgcaaggtgtccggcgacaccatcaccttctactacatgcacttcgtgaagcagaggccaggacagggcctggaatggatcggcagaatcgaccctgaggacgagagcaccaagtacagcgagaagttcaagaacaaggccaccctgaccgccgacaccagcagcaacaccgcctacctgaagctgtctagcctgacctccgaggacaccgccacctacttttgcatctacggcggctactacttcgactactggggccagggcgtgatggtcaccgtgtccagcatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgttcaacgagctgaacctgggcagacgggaggaattcgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgttccagggcctgtccaccgccaccaaggacaccttcgacgccctgcacatgcagaccctggcccccaga

SEQ ID NO: 78 - DNA, 582 bp, synthetic construct
  source 1..582: mol_type = other DNA
atggtgttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcagactacaaagaccatgacggtgattataaagatcatgacatcgattacaaggatgacgatgacaagtaa

SEQ ID NO: 79 - DNA, 600 bp, synthetic construct
  source 1..600: mol_type = other DNA
atgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 80 - DNA, 582 bp, synthetic construct; identical to SEQ ID NO: 78
  source 1..582: mol_type = other DNA

SEQ ID NO: 81 - RNA, 1410 nt, synthetic construct; identical to SEQ ID NO: 73
  source 1..1410: mol_type = other RNA

SEQ ID NO: 82 - RNA, 717 nt, synthetic construct
  source 1..717: mol_type = other RNA
atggacatgagggtaccagcacagttgctgggactgttactgctctggctctccggtgctcgctgtgatatccagatgacacagagtccctctagcctgagcgcatcagtcggagaccgcgtgacaatcacctgcagagctagccagggcataagaaactatctcgcatggtaccagcagaaacccggaaaggcccctaagttgctgatttatgctgcttctacactacaaagcggcgtgcccagccgattctccgggtctgggagtggcacggactttactcttactatctcctcactgcaaccagaggatgtagccacgtactattgtcagcgttacaatcgggccccctacacatttggccagggaaccaaagttgaaatcaaaaggactgtggcggccccttcggttttcattttcccaccatcagacgagcaactcaagtctggtacagcatccgtcgtctgccttctcaataacttctacccgagggaggccaaagtccagtggaaagttgacaatgccctgcagagtgggaactcacaggaatccgtgaccgaacaagactcaaaggatagcacctatagtctaagcagcaccttgactctgtcgaaggccgattatgaaaagcacaaagtgtacgcgtgtgaggtgacccatcagggcctgtcctcccctgtgaccaagagttttaaccggggggagtgctgataatag

SEQ ID NO: 83 - RNA, 1383 nt, synthetic construct
  source 1..1383: mol_type = other RNA
atggagttcggtctgtcttgggtctttcttgtcgctttgtttcggggagttcagtgtgaagtgcaactggtgcagtcaggagccgaagtgaaaaagccgggtgctagcgtgaaagtgtcctgcaaagcgtcgggttacaccctgacttcctacgggatttcgtgggtccgccaagcccctggccaaggactggaatggatgggctgggtgtcgttctataacggcaacaccaactacgcgcagaagcttcagggacgggggactatgaccaccgacccgtccacttccaccgcctatatggagttgcgcagcctccggtccgacgatactgccgtgtactactgcgcaaggggttacggaatggacgtctggggacagggtactaccgtgacggtgtcgtcagccagcaccaagggcccatccgtattcccactggccccttgctcccgctccacctccgaatccaccgccgccctgggctgcctcgtgaaggattacttccccgaacctgtgaccgtgtcctggaattcaggggccctgactagcggcgtgcacactttcccggcggtgctccagtcgagcggcctgtacagcctctcgagtgtcgtgaccgtgccctcctccaacttcggaacccagacttacacatgcaacgtcgaccacaagccttccaacaccaaggtcgacaagactgtggagagaaagtgctgcgtggaatgcccgccatgccctgcccctcccgtggcgggaccgtcggtgttcctgttcccgccaaagccgaaggacaccctgatgatcagcagaactcccgaagtgacctgtgtggtggtcgatgtgtctcatgaggaccccgaggtgcagttcaattggtacgtggacggggtggaggtccataacgcaaagaccaagccccgcgaggaacagttcaactccaccttccgggtggtgtcagtgctgacagtcgtgcaccaagattggctgaacggaaaggagtacaagtgcaaagtgtccaacaagggcctgcctgccccgattgaaaagaccatctccaagactaagggccagccgagggagccccaggtctacaccctgccgccctcacgggaagaaatgactaagaaccaagtgtccctgacctgtctggtcaagggcttttacccctcggacatcgccgtggagtgggagtcaaacggacagccagagaacaactacaagaccacccctcctatgctggacagcgacggaagcttcttcctgtactccaagctcaccgtggataagtccagatggcagcagggaaacgtgttctcgtgttccgtgatgcacgaagctctccacaaccactacacgcaaaagtccctcagcctgagcccggggaaataa

SEQ ID NO: 84 - RNA, 714 nt, synthetic construct
  source 1..714: mol_type = other RNA
atgaagtacctcttgcctactgctgccgccggactcttgctgcttgccgcccaacccgctatggccgaatcggcgctgacccagcctgcctctgtctccggttccccgggacagtcaattaccatcagctgcactggaacctcctccgacgtgggcggctacaattcagtgtcgtggtaccagcagcaccctgggaaggcacccaagctgatgatctacgaggtgtccaaccgcccaagcggagtgtcaaaccggttctccggaagcaaatcaggaaacaccgcctccctgactatcagtgggctgcaggccgaggacgaagcagattactactgcaactcgtacacctccactagcatggtgttcggcggcgggactaagctgaccgtgctgggacagcccaaggcggccccctccgtgactctgttcccgccgtcgtccgaggaactgcaggctaacaaggccaccctcgtgtgcctgattagcgacttttaccctggcgccgtgaccgtggcctggaaggcggattcgtcgccggtcaaggccggtgtcgaaactaccaccccgtccaagcagtccaacaacaaatacgccgcgagcagctatctgagcctcacccccgaacaatggaagtcgcaccgctcctactcctgccaagtcacccatgagggttccaccgtggaaaagacggtggcaccaacagagtgttcctaa

SEQ ID NO: 85 - RNA, 1380 nt, synthetic construct
  source 1..1380: mol_type = other RNA
atggaatttggactgtcttgggtttttctggtagctctgtttcgtggagtgcaatgcgaagtccagctcgtgcagtctggagcagaggtcaaaaaaccaggggctagcgtgaaagttagttgcaaggcttcagggtacacgttgacctcctacggcatctcctgggtgagacaagcacccgggcaaggactagagtggatgggttgggtcagtttctacaacggcaataccaactacgcccagaagcttcaagggcgtggtacaatgacgactgacccaagcaccagcacggcctatatggaactcagaagccttaggtctgacgacacagcagtttactattgtgcgcgaggttacggcatggatgtgtgggggcaggggaccacagtgacagtgagttccgccagcactaaggggccaagcgtctttcccctggccccgtgctcaaggtcaaccagtgagagtaccgccgccctgggctgcttagtgaaagattatttccccgagccagtgacggtgagttggaactctggcgccctcacatccggagtacacactttcccggcggtgctgcagtctagtggactgtattctttaagcagtgtcgtgactgtgccttctagcaacttcggcactcagacatacacatgcaacgttgaccacaaaccctccaatacaaaggtggataaaaccgttgaacggaaatgttgtgtggaatgtccaccgtgtccagctccaccagttgctggacctagcgtctttttgttccctcctaagcccaaagatactctgatgatctcgcggacccctgaggttacctgcgtagtggtagacgtgtcacatgaggatcccgaagttcagttcaattggtatgtcgatggcgtagaggtccacaacgctaagactaagccaagggaggaacagtttaactctacatttcgggtggttagcgtcctaacggtggtccaccaggattggctgaatggaaaggagtacaagtgcaaggtgtcaaacaaggggctccctgcacctattgaaaagaccatctccaagactaagggtcagccacgcgagccccaagtatacacccttcctccctcccgcgaagagatgaccaaaaatcaggtgtcactcacctgcttggtcaagggcttttatccctccgacatagcagtagaatgggagtccaacggacagcccgaaaacaattataaaacaaccccgcccatgctggactcagacggctctttcttcctctactcgaaactgacagtggacaagtcaagatggcaacagggcaatgtcttttcctgttcggtgatgcacgaggccctgcataatcattatactcagaaaagcctgtccctgagccctggaaag

SEQ ID NO: 86 - RNA, 711 nt, synthetic construct
  source 1..711: mol_type = other RNA
atgaagtatctactgcctaccgctgcagctgggctgttgcttctggctgctcaaccagccatggctgagagtgctctgacccagccagcaagtgtgagcggaagccctggccagtcaataaccatctcctgtactggcacgtcaagtgacgtggggggttataatagcgtctcatggtaccagcaacaccccgggaaggcgcccaagctgatgatctacgaagttagcaatcggccgagcggagtatctaataggttctcgggctcaaagtctggaaacacggcatcactgacaattagtggcctccaggcagaggatgaagctgactactattgtaactcctatactagtaccagtatggtgtttggcggagggaccaaactgacagtcttagggcagcctaaggccgccccctccgtcacactgttcccaccttccagcgaagaactacaagcaaacaaagctacattggtctgcctcatctccgacttttacccgggtgccgttaccgtggcgtggaaagccgattctagccctgtgaaggccggcgtagagaccacaaccccatccaagcagagcaacaataaatacgccgcctcctcatacctctcgcttactcccgagcaatggaaaagccacagaagctatagctgccaggttactcatgagggatctactgtggaaaagacagtggctccaaccgagtgctcc

SEQ ID NO: 87 - protein, 460 aa, synthetic construct
MEFGLSWVFLVALFRGVQCEVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 88 - protein, 237 aa, synthetic construct
MKYLLPTAAAGLLLLAAQPAMAESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 89 - RNA, 11 nt, synthetic construct
  source 1..11: mol_type = other RNA
gggatcctacc

SEQ ID NO: 90 - RNA, 30 nt, synthetic construct
  source 1..30: mol_type = other RNA
aaaaaaaaaaaaaaaaaaaaaaaaaaaaaa

SEQ ID NO: 91 - RNA, 70 nt, synthetic construct
  source 1..70: mol_type = other RNA
aaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0315401 A1
(43) Pub. Date: Oct. 15, 2026

(54) DELIVERY OF POLYNUCLEOTIDES FROM LIPID NANOPARTICLES COMPRISING RNA AND IONIZABLE LIPIDS

(71) Applicant: SAIL BIOMEDICINES, INC., Cambridge, MA (US)

(72) Inventors: Munir MOSAHEB, Cambridge, MA (US); Siddharth PATEL, Cambridge, MA (US); Erin TROY, Cambridge, MA (US); Yasmeen FARRA, Cambridge, MA (US); Maya GOUGH, Cambridge, MA (US); Alaina HOWE, Cambridge, MA (US); Sricharani BALMURI, Cambridge, MA (US); Sanmit ADHIKARI, Cambridge, MA (US)

(21) Appl. No.: 19/476,003

(22) PCT Filed: Apr. 18, 2024
(86) PCT No.: PCT/US24/25134

§ 371 Date: Oct. 16, 2025

Related U.S. Application Data
(60) Provisional application No. 63/460,586, filed on Apr. 19, 2023.
(60) Provisional application No. 63/502,522, filed on May 16, 2023.
(60) Provisional application No. 63/547,630, filed on Nov. 7, 2023.

(51) Int. Cl. A61K 9/51; A61K 9/00; A61K 9/1272; C12N 15/88

(52) U.S. Cl. CPC A61K 9/5123; A61K 9/0019; A61K 9/1272; C12N 15/88

(57) ABSTRACT

Provided herein is a method for delivering an RNA composition to a subject for in vivo production of a protein or a peptide in the subject, comprising administering to the subject an RNA composition comprising a polynucleotide that encodes a protein or a peptide, formulated within (a) a plurality of lipid nanoparticles (LNP) comprising synthetic structural lipids and an ionizable lipid, or (b) a lipid reconstructed natural messenger packs (LNMPs) comprising natural lipids and an ionizable lipid. Also provided herein are methods for treating diseases or disorders associated with the gastrointestinal tract, stomach, small or large intestine, mesenteric lymph node, pancreas, colon or rectum, caecum, and/or spleen, comprising orally or enterally administering the RNA composition described herein.

### CROSS-REFERENCE TO RELATED APPLICATIONS

This application claims benefit of priority to U.S. Provisional Application No. 63/460,586, filed Apr. 19, 2023; U.S. Provisional Application No. 63/502,522, filed May 16, 2023; and U.S. Provisional Application No. 63/547,630, filed Nov. 7, 2023, all of which are herein incorporated by reference in their entirety.

### BACKGROUND

The use of polynucleotides as therapeutics is a new and emerging field. A need therefore exists for developing an oral and enteral polynucleotide delivery system for a more effective, easily scalable, and stable delivery of RNA therapeutics that will help with patient's adherence to the treatment. Delivery methods targeting the gastrointestinal tract and surrounding immune systems are also needed.

### SUMMARY OF THE INVENTION

In one aspect, provided herein is a method for delivering an RNA composition to a subject for in vivo production of a protein or a peptide in the subject. The method comprises administering to the subject (e.g., by oral, enteral or intravenous delivery) an RNA composition comprising one or more polynucleotides (e.g., an mRNA or circRNA) that encodes a protein or a peptide that is formulated within (a) a plurality of lipid nanoparticles (LNP) comprising synthetic structural lipids and an ionizable lipid or within (b) a complex lipid particle (CLP), such as a lipid reconstructed natural messenger packs (LNMPs), comprising natural lipids and an ionizable lipid.

The ionizable lipid has two or more of the characteristics listed below:
(i) at least one ionizable amine;
(ii) at least three lipid tails, wherein each of the lipid tails is at least six carbon atoms in length;
(iii) a pKa of about 4.5 to about 7.5;
(iv) an ionizable amine and a heterorganic group separated by a chain of at least two atoms; and
(v) an N:P ratio of at least 3.

In some embodiments, the RNA composition is administered orally or enterally. In some embodiments, the RNA composition is administered systemically (e.g., intravenously).

In another aspect, provided herein is a method for delivering an RNA composition to the lymphatic transport system and bypassing the liver. The method comprises administering to the subject the RNA composition comprising one or more polynucleotides (e.g., an mRNA or circRNA) encoding one or more polypeptides formulated within a LNP comprising synthetic structural lipids and an ionizable lipid, or a CLP, such as a LNMP, comprising natural lipids and an ionizable lipid. The ionizable lipid has two or more of the characteristics listed below:
(i) at least 1 ionizable amine;
(ii) at least 3 lipid tails; wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.

In another aspect, provided herein is a method of treating diseases or disorders associated with the gastrointestinal tract, stomach, small or large intestine, mesenteric lymph node, pancreas, colon or rectum, caecum, and/or spleen. The method comprises orally administering an RNA composition having one or more polynucleotides encoding one or more polypeptides. The one or more polynucleotides are formulated within a plurality of lipid nanoparticles (LNP) comprising synthetic structural lipids and an ionizable lipid, or a CLP such as lipid reconstructed natural messenger packs (LNMPs) comprising natural lipids and an ionizable lipid. The ionizable lipid has two or more of the characteristics listed below:
(i) at least one ionizable amine;
(ii) at least three lipid tails, wherein each of the lipid tails is at least six carbon atoms in length;
(iii) a pKa of about 4.5 to about 7.5;
(iv) an ionizable amine and a heterorganic group separated by a chain of at least two atoms; and
(v) an N:P ratio of at least 3.

In some embodiments, the disease or disorder is pancreatitis, inflammatory bowel disease (IBD), Crohn's disease, colorectal cancer, or ulcerative colitis.

In another aspect of the invention, provided herein is a method for in vivo delivery of an antibody to a subject, comprising:
administering systemically to a subject an RNA composition comprising one or more polynucleotides (e.g., an mRNA or circRNA) encoding an antibody, formulated within a complex lipid particle (CLP), such as a lipid reconstructed natural messenger packs (LNMPs) comprising natural lipids and an ionizable lipid, wherein the ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.

In an alternative aspect of the invention, provided herein is a method for in vivo delivery of an antibody to a subject, comprising:
administering systemically to a subject an RNA composition comprising one or more polynucleotides (e.g., an mRNA or circRNA) encoding an antibody, formulated within a plurality of lipid nanoparticles (LNPs) comprising synthetic structural lipids and an ionizable lipid, wherein the ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.

In another aspect, provided herein is an oral vaccination composition (e.g., for viral or bacterial infection or for anti-tumor), comprising an RNA composition having one or more polynucleotides (e.g., an mRNA or circRNA) encoding one or more polypeptides formulated within (a) a plurality of lipid nanoparticles (LNP) comprising synthetic structural lipids and comprising an ionizable lipid, or

(b) a lipid reconstructed natural messenger packs (LNMPs) comprising natural lipids and an ionizable lipid. The ionizable lipid has two or more of the characteristics listed below:
(i) at least one ionizable functional group amine;
(ii) at least three lipid tails, wherein each of the lipid tails is at least six carbon atoms in length;
(iii) a pKa of about 4.5 to about 7.5;
(iv) an ionizable amine functional group and a heterorganic functional group separated by a chain of at least two atoms; and
(v) an N:P ratio of at least 3, wherein the mRNA composition is formulated in an oral dosage form. In some embodiments, the polypeptide is an antigenic polypeptide derived from an infectious agent that causes a viral infection or bacterial infection. In some embodiments, the antigenic polypeptide is a corona virus.

All the embodiments discussed below are applicable to all the above aspects.

In some embodiments, the polynucleotide is an mRNA or circRNA. In some embodiments, the mRNA or circRNA is derived from (a) a DNA molecule; or (b) an RNA molecule, wherein T is substituted with U.

In some embodiment, the polynucleotide encodes a protein, peptide, or polypeptide comprising an antibody. In some embodiments, the antibody is a therapeutic. In one embodiment, the antibody is TNF inhibitor or PCSK9 inhibitor. In one embodiment, the RNA composition comprising one or more polynucleotides (e.g., an mRNA or circRNA) encoding the antibody is administered at least one time, optionally two or more times.

In some embodiments, the in vivo production of the protein or the peptide (or polypeptide) occurs in the subject's stomach, small intestine, mesenteric lymph node, pancreas, colon, caecum, and/or spleen. In some embodiments, administering the RNA composition results in expression of the protein or the peptide (or polypeptide) detectable in one or more organs in the subject at least about 6 hours, about 12 hours, about 24 hours, about 48 hours, about 72 hours, or about 96 hours after administration. In some embodiments, the organs are present along the transit route of the digestive track, and the RNA composition's access to the organs occurs through the lymphatic transport system.

In some embodiments, the protein or the peptide (or polypeptide) encoded by the polynucleotide (e.g., polyribonucleotide, mRNA, or circRNA) is detectable in the subject's mesenteric lymph node, pancreas, stomach, colon, spleen, and/or small intestine (e.g., villi, Peyer's patches) at least about 6 hours, about 12 hours, about 24 hours, about 48 hours, about 72 hours, or about 96 hours after administration. In some embodiments, the protein or the peptide (or polypeptide) encoded by the polynucleotide is not detectable in the subject's liver at least about 6 hours, about 12 hours, about 24 hours, about 48 hours, about 72 hours, or about 96 hours after administration.

In some embodiments, the RNA composition is formulated within a plurality of lipid nanoparticles (LNP) comprising synthetic structural lipids and the ionizable lipid.

In some embodiments, the RNA composition is formulated within a LNMP comprising natural lipids and the ionizable lipid.

In some embodiments, the RNA composition is formulated within a CLP. In some embodiments, the CLP is LNMP. Thus, all the embodiments below describing the features relating to LNMP and LNMP formulation are applicable to CLP and CLP formulation.

In some embodiments, the polynucleotide is encapsulated by the LNPs or LNMPs. In some embodiments, the polynucleotide is embedded on the surface of the LNPs or LNMPs. In some embodiments, the polynucleotide is conjugated to the surface of the LNPs or LNMPs.

In some embodiments, the RNA composition is administered to the subject in a delayed-release pharmaceutical dosage form comprising (a) a therapeutically effective amount of a polynucleotide (e.g., a mRNA or circRNA); (b) a bile salt or bile acid; and (c) at least one surfactant selected from hydrophilic surfactants, lipophilic surfactants, and mixtures thereof.

In some embodiments, the RNA composition is administered in the form of a capsule (e.g., a starch capsule, a cellulosic capsule, a hard gelatin capsule, or a soft gelatin capsule). In some embodiments, the RNA composition is administered in the form of a tablet or caplet. In some embodiments, the capsule, tablet, or caplet contains an enteric coating. In some embodiments, the RNA composition is administered in the form of a plurality of particles, granules, beads, pellets, or mixtures thereof.

In some embodiments, the RNA composition comprises:
one or more polynucleotides (e.g., an mRNA or circRNA) encoding one or more polypeptides, formulated within a CLP, such as a lipid reconstructed natural messenger pack (LNMP) comprising natural lipids and an ionizable lipid,
wherein the ionizable lipid has two or more of the characteristics listed below:
(i) at least one ionizable amine functional groups;
(ii) at least three lipid tails, wherein each of the lipid tails is at least six carbon atoms in length;
(iii) a pKa of about 4.5 to about 7.5;
(iv) an ionizable amine functional group and a heterorganic functional group separated by a chain of at least two atoms; and
(v) an N:P ratio of at least 3.

In some embodiments, the ionizable lipid may be 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), or ALC-315. In one embodiment, the ionizable lipid included is C12-200.

In some embodiments, the ionizable lipid included is

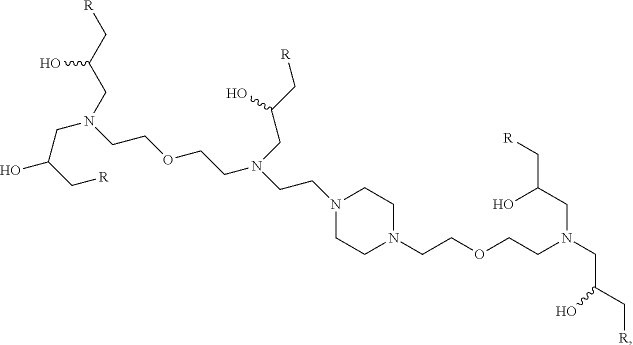

wherein R is C8-C14 alkyl group.

In all these aspects, the ionizable lipid may be selected from one of the following groups of compounds:
i) a compound of formula

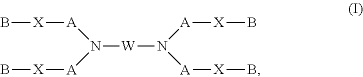

a pharmaceutically acceptable salt thereof, or a stereoisomer of any of the foregoing, wherein:
each A is independently C₁-C₁₆ branched or unbranched alkyl or C₁-C₁₆ branched or unbranched alkenyl, optionally substituted with heteroatom or substituted with OH, SH, or halogen;
each B is independently C₁-C₁₆ branched or unbranched alkyl or C₁-C₁₆ branched or unbranched alkenyl, optionally substituted with heteroatom or substituted with OH, SH, or halogen;
each X is independently a biodegradable moiety; and
W is

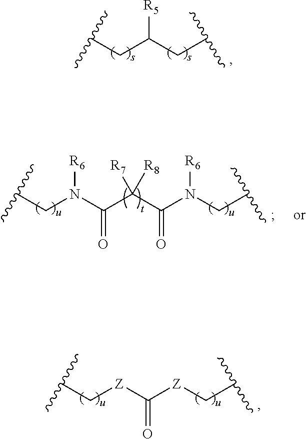

wherein:
   R₅ is OH, SH, or NR₁₀R₁₁;
   each R₆ is independently H, C₁-C₃ branched or unbranched alkyl, C₂-C₃ branched or unbranched alkenyl, or cycloalkyl;
   each R₇ and each R₈ is independently H, C₁-C₃ branched or unbranched alkyl, C₂-C₃ branched or unbranched alkenyl, halogen, OH, SH, or NR₁₀R₁₁, wherein each R₁₀ and R₁₁ is independently H, C₁-C₃ alkyl, or R₁₀ and R₁₁ are taken together to form a heterocyclic ring; R₇ and R₈ are taken together to form a ring;
   each s is independently 1, 2, 3, 4, or 5;
   each u is independently 1, 2, 3, 4, or 5;
   t is 1, 2, 3, 4 or 5;
   each Z is independently absent, O, S, or NR₁₂, wherein R₁₂ is H, C₁-C₇ branched or unbranched alkyl, or C₂-C₇ branched or unbranched alkenyl, and
   Q is O, S, or NR₁₃, wherein each R₁₃ is H, or C₁-C₅ alkyl;
ii) a compound of formula

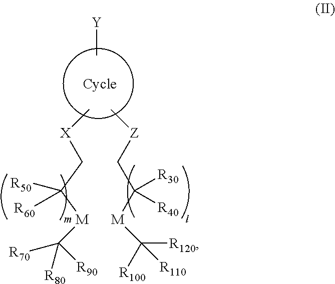

a pharmaceutically acceptable salt thereof, or a stereoisomer of any of the foregoing, wherein:

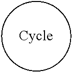

is cyclic or heterocyclic moiety;
Y is alkyl, hydroxy, hydroxyalkyl or

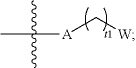

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
iii) a compound of formula

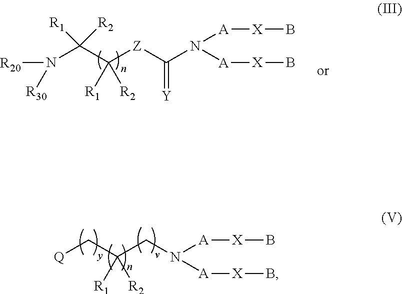

pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or R₁₀ and R₁₁ are taken together to form a heterocyclic ring;
n is 0, 1, 2, 3 or 4;
Y is O or S;
Z is absent, O, S, or N(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
v is 0, 1, 2, 3, or 4;
y is 0, 1, 2, 3, or 4;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen; and
each X is independently a biodegradable moiety; and
iv) a lipid comprising at least one head group and at least one tail group of formula (TI)
or (TI′):

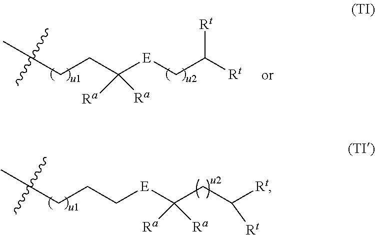

(TI′), a pharmaceutically acceptable salt thereof, or a stereoisomer of any of the foregoing,
wherein:
E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl;
R₁₃ is branched or unbranched C₃-C₁₀ alkyl;
r is 1, 2, 3, 4, or 5;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;

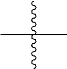

represents the bond connecting the tail group to the head group; and
wherein the lipid has a pKa from about 4 to about 8.

In some embodiments, the ionizable lipid is a compound of group i), represented by a formula of

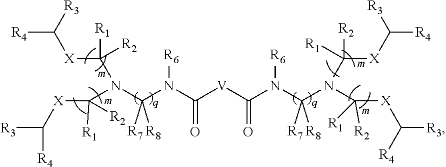

pharmaceutically acceptable salts thereof, and stereoisomers of any of the foregoing, wherein:
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each q is independently 2, 3, 4, or 5;
V is branched or unbranched C₂-C₁₀ alkylene, C₂-C₁₀ alkenylene, C₂-C₁₀ alkynylene, or C₂-C₁c heteroalkylene, optionally substituted with one or more OH, SH, and/or halogen groups;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each v is independently 0, 1, 2, 3, 4, or 5, and R₁₇ is OH, SH, or N(CH₃)₂; and
each m is independently 1, 2, 3, 4, 5, 6, 7, 8, 9 or 10. In some embodiments, V is a branched or unbranched C₂-C₃ alkylene, and each R₆ is independently H or methyl.

In some embodiments, the ionizable lipid is a compound of group i), represented by a formula of

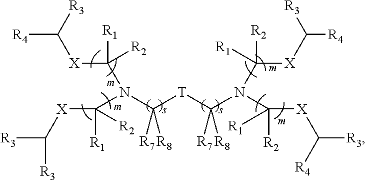

pharmaceutically acceptable salts thereof, and stereoisomers of any of the foregoing, wherein:
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each s is independently 1, 2, 3, 4, or 5;
T is —NHC(O)O—, —OC(O)NH—, or a divalent heterocyclic optionally substituted with one or more
   —(CH₂)ᵥOH, —(CH₂)ᵥSH, —(CH₂)ᵥ-halogen groups,
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein R₁₇ is OH, SH, or N(CH₃)₂;
each v is independently 0, 1, 2, 3, 4, or 5; and
each m is independently 1, 2, 3, 4, 5, 6, 7, 8, 9 or 10. In some embodiments, T is a divalent piperazine or a divalent dioxopiperazine.

In some embodiments, in the above formulas for group i), X is —OCO—, —COO—, —NHCO—, or —CONH—.

In some embodiments, the ionizable lipid is a compound of group ii), represented by one of the following formulas:

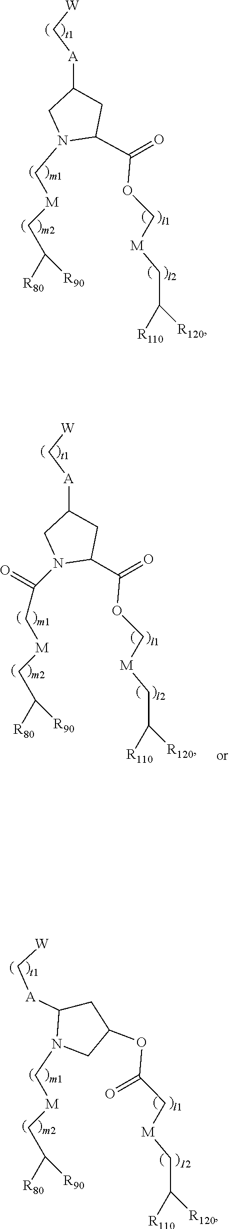

wherein:
A is absent, —O—, —N(R⁷)—, —O-alkylene-, -alkylene-O—, —OC(O)—, —C(O)O—,
—N(R⁷)C(O)—, —C(O)N(R⁷)—, —N(R⁷)C(O)N(R⁷)—, —S—, —S—S—, or a bivalent heterocycle;
each R⁷ is independently H, alkyl, alkenyl, cycloalkyl, hydroxy, hydroxyalkyl, or aminoalkyl;
t1 is an integer from 0 to 10;
W is hydroxyl, substituted or unsubstituted hydroxyalkyl, substituted or unsubstituted amino, substituted or unsubstituted aminocarbonyl, or substituted or unsubstituted heterocyclyl or heteroaryl;
each M is independently a biodegradable moiety;
each m1 is independently an integer from 3 to 6,
each l1 is independently an integer from 4 to 8,
m2 and l2 are each independently an integer from 0 to 3,
R₈₀ and R₉₀ are each independently unsubstituted C₅-C₈ alkyl or alkenyl; or R₈₀ is H or unsubstituted C₁-C₄ alkyl or alkenyl, and R₉₀ is unsubstituted C₅-C₁₁ alkyl or alkenyl; and
R₁₁₀ and R₁₂₀ are each independently unsubstituted C₅-C₈ alkyl or alkenyl; or R₁₁₀ is H or unsubstituted C₁-C₄ alkyl or alkenyl, and R₁₂₀ is unsubstituted C₅-C₁₁ alkyl or alkenyl. In some embodiments, M is —OC(O)— or —C(O)O—;

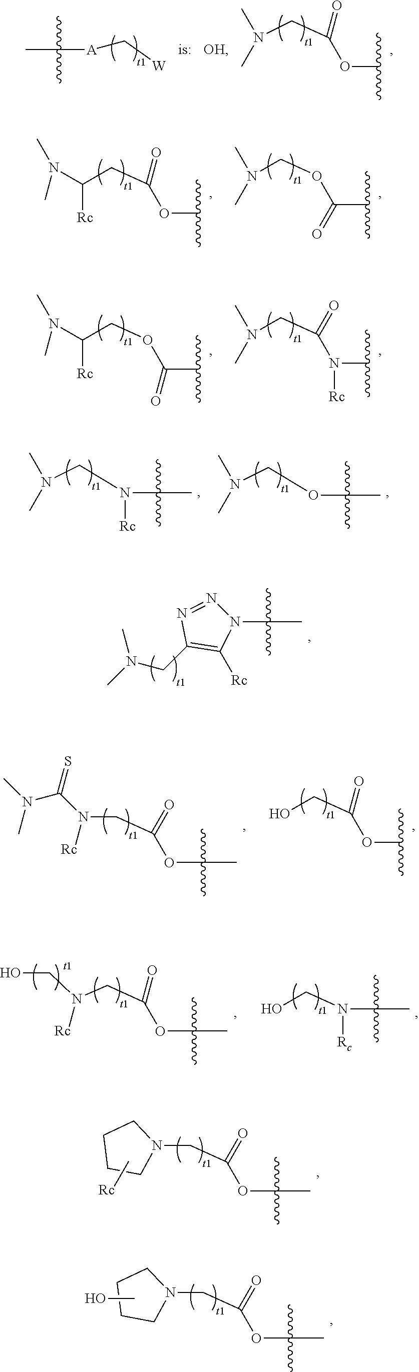

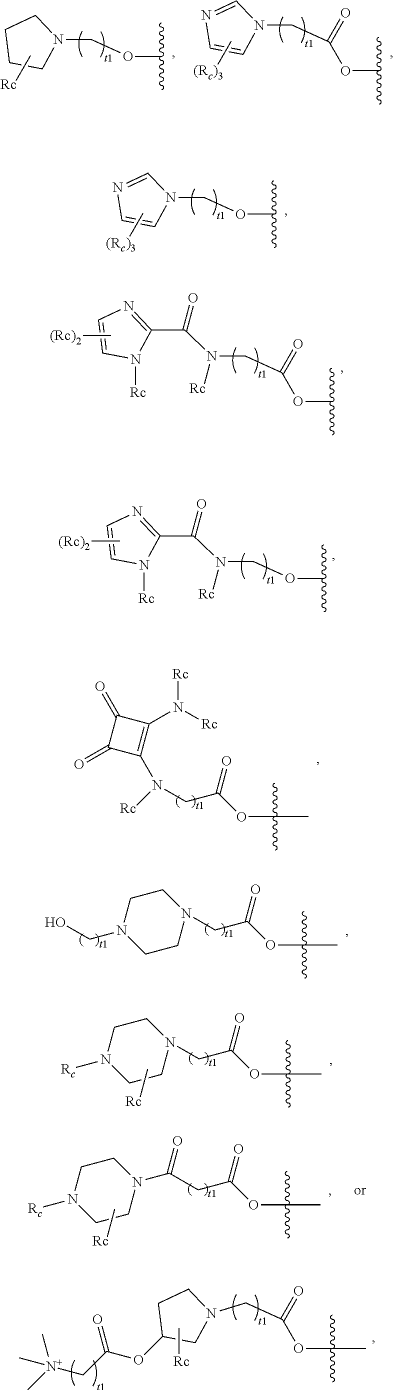

each R^{c} is independently H or C₁-C₃ alkyl; and
each t1 is independently 1, 2, 3, or 4.

In some embodiments, the ionizable lipid is a compound of group iii), wherein R₁ and R₂ are each H, or each R₁ is H, and one of the R₂ variables is OH; and X is —OC(O)— or —C(O)O—. In some embodiments, the ionizable lipid is a compound of group iii), represented by formula III, wherein R₂₀ and R₃₀ are each independently H or C₁-C₃ branched or unbranched alkyl; or R₂₀ and R₃₀ together with the adjacent N atom form a 3 to 7 membered cyclic ring, optionally substituted with R^{a}; R^{a} is H or OH; Z is absent, S, O, or NH; and n is 0, 1, or 2. In some embodiments, the ionizable lipid is a compound of group iii), represented by formula V.

In some embodiments, the ionizable lipid is a compound of group iv), wherein the lipid comprises at least one head group and at least one tail group, wherein:
the tail group has a structure of formula (TI) or formula TI′

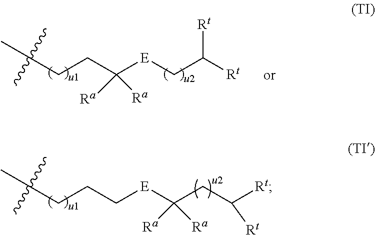

and
the head group has a structure of one of the following formulas:

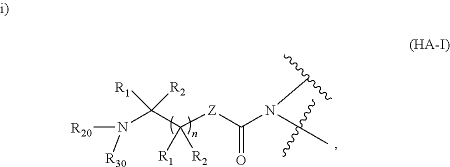

wherein:
   R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
   R₂₀ and R₃₀, together with the adjacent N atom, form a 3 to 7 membered heterocyclic or heteroaromatic ring containing one or more heteroatoms, optionally substituted with one or more OH, SH, halogen, alkyl, or cycloalkyl groups;
   each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
   each of R₁₀ and R₁₁ is independently H, C₁-C₃ branched or unbranched alkyl, C₂-C₃ branched or unbranched alkenyl; or R₁₀ and R₁₁ together form a heterocyclic ring;
   n is 0, 1, 2, 3 or 4; and
   Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, or SH;

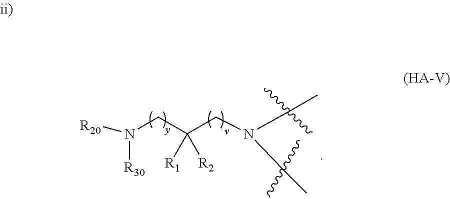

wherein:
   R₁ is H, C₁-C₃ alkyl, OH, halogen, SH, or NR₁₀R₁₁;
   R₂ is OH, halogen, SH, or NR₁₀R₁₁; or R₁ and R₂ can be taken together to form a cyclic ring;
   R₁₀ and R₁₁ are each independently H or C₁-C₃ alkyl; or R₁₀ and R₁₁ can be taken together to form a heterocyclic ring;
   R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
   each of v and y is independently 1, 2, 3, or 4;

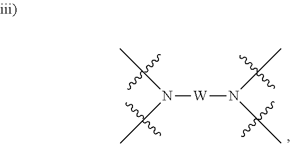

wherein W is

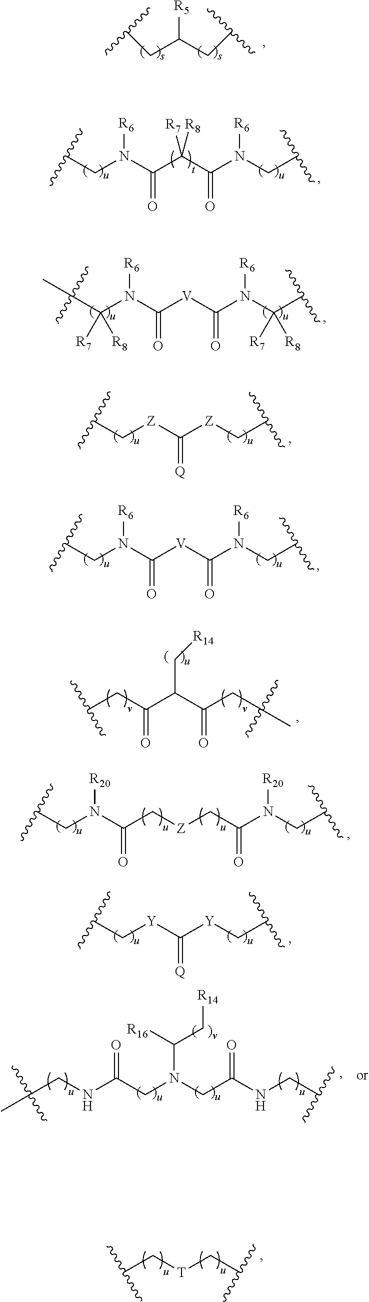

wherein
   R₅ is OH, SH, (CH₂)ₛOH, or NR₁₀R₁₁;
   each R₆ is independently H, C₁-C₃ branched or unbranched alkyl, C₂-C₃ branched or unbranched alkenyl, or cycloalkyl;
   each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
   each R₂₀ is independently H, or C₁-C₃ branched or unbranched alkyl;
   R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
   R₁₆ is H, ═O, ═S, or CN;
   each of s, u, and t is independently 1, 2, 3, 4, or 5;
   each v is independently 0, 1, 2, 3, 4, or 5;
   each Y is a divalent heterocyclic;
   each Z is independently absent, O, S, or NR₁₂, wherein R₁₂ is H, C₁-C₇ branched or unbranched alkyl, or C₂-C₇ branched or unbranched alkenyl;
   Q is O, S, CH₂, or NR₁₃, wherein each R₁₃ is H, or C₁-C₅ alkyl;
   V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
   T is —NHC(O)O—, —OC(O)NH—, or a divalent heterocyclic; and
iv)

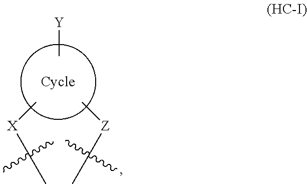

wherein:

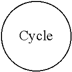

is cyclic or heterocyclic moiety;
   Y is alkyl, hydroxy, hydroxyalkyl,

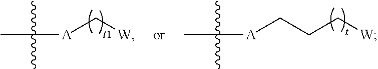

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t is 0, 1, 2, or 3;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
wherein the lipid has a pKa from about 4 to about 8.

In some embodiments, the ionizable lipid is a compound of group iv), and wherein at least one tail group of the lipid has one of the following formulas:

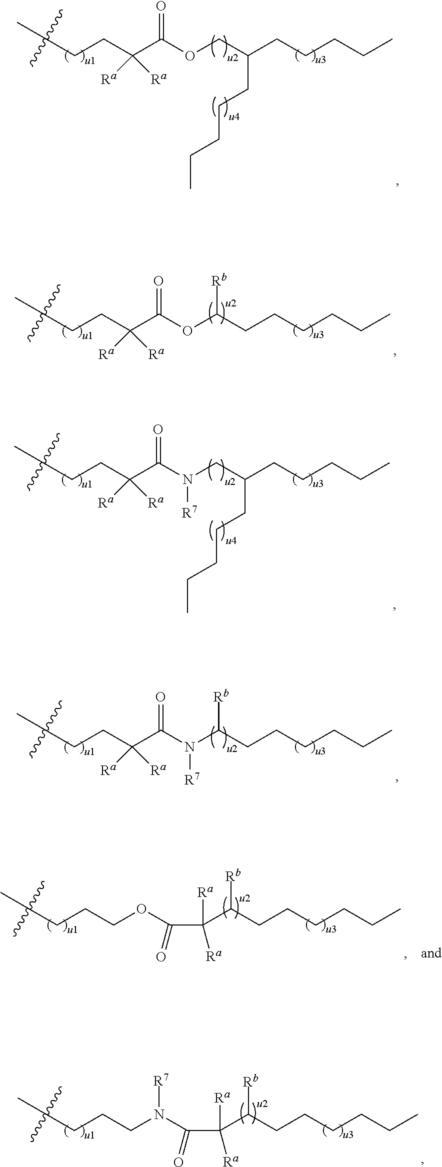

wherein:
R⁷ is each independently H or methyl;
R^{b} is in each occasion independently H or C₁-C₄ alkyl;
u1 and u2 are each independently 0, 1, 2, 3, 4, 5, 6, or 7; and
u3 and u4 are each independently 0, 1, 2, 3, 4, 5, 6, or 7; and
   the head group has a structure of one of the following formulas:

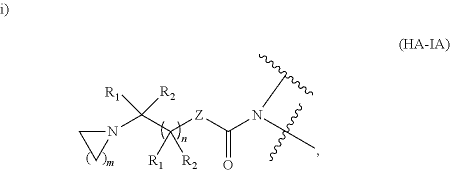

wherein m is 1, 2, 3, 4, 5, 6, 7 or 8;

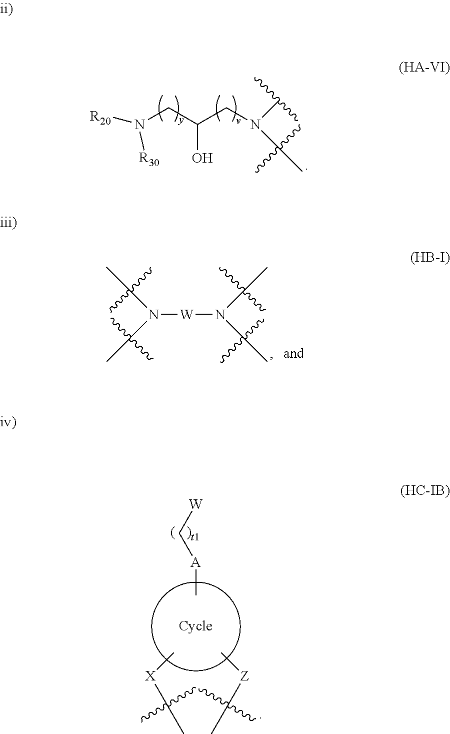

In some embodiments, at least one tail group has the structure of formula (TII), (TIII), (TIV), (TV), (TII′), and/or (TIII′), wherein u1 is 3-5, u2 is 0-3, u3 and u4 are each independently 1-7, and R^{a} is each independently methyl.

In some embodiments, the tail group has the structure of formula (TII) or formula (TIII), wherein each R^{a} is methyl; u1 is 3-5, u2 is 0-3; and u3 and u4 are each independently 1-4.

In some embodiments, the head group has the structure of one of the following formulas

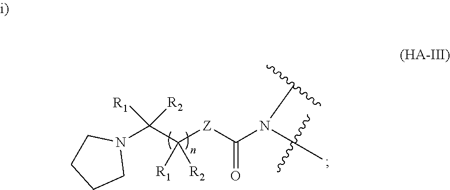

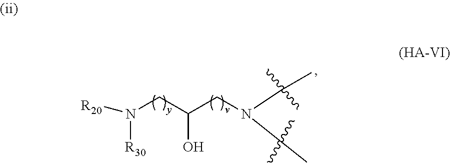

wherein each R₂₀ and R₃₀ are independently C₁-C₃ alkyl,

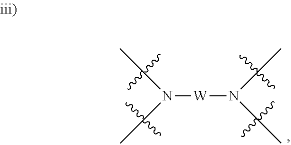

wherein:
W is

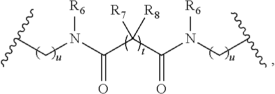

wherein:
   each R₆, R₇, and R₈ are independently H or methyl; and
   each of u and t is independently 1, 2, or 3; or
W is

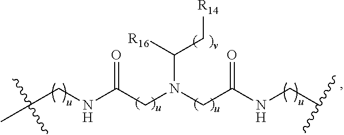

wherein:
   R₁₆ is H or ═O;
   R₁₄ is a nitrogen-containing 5- or 6-membered heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H or C₁-C₃ alkyl; and
   each of u and v is independently 1, 2, or 3; or
W is

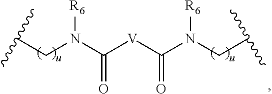

wherein:
   each R₆ is independently H or methyl;
   each u is independently 1, 2, or 3; and
   V is C₂-C₆ alkylene or C₂-C₆ alkenylene; or
W is

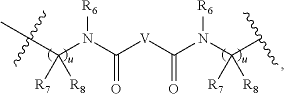

wherein:
   each R₆ is independently H or methyl;
   each R₇ is independently H;
   each R₈ is methyl;
   each u is independently 1, 2, or 3; and
   V is C₂-C₆ alkylene or C₂-C₆ alkenylene; or
W is

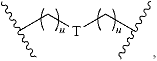

wherein:
   each u is independently 1, 2, or 3; and
   T is a divalent nitrogen-containing 5- or 6-membered heterocyclic; or
W is

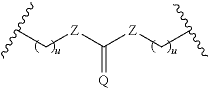

wherein:
each u is independently 1, 2, or 3;
Q is O;
each Z is independently NR₁₂; and
R₁₂ is H or C₁-C₃ alkyl; and
iv)

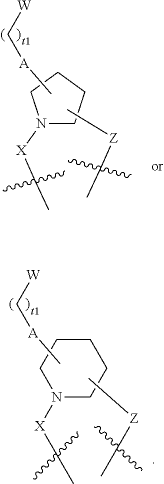

wherein:
   W is hydroxyl, substituted or unsubstituted hydroxyalkyl, one of the following moieties:

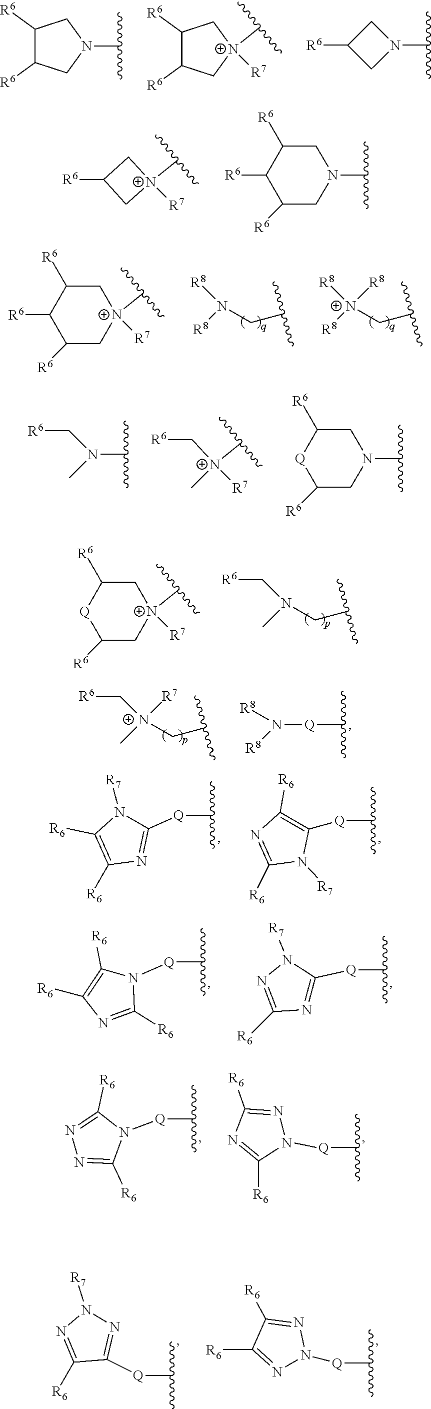

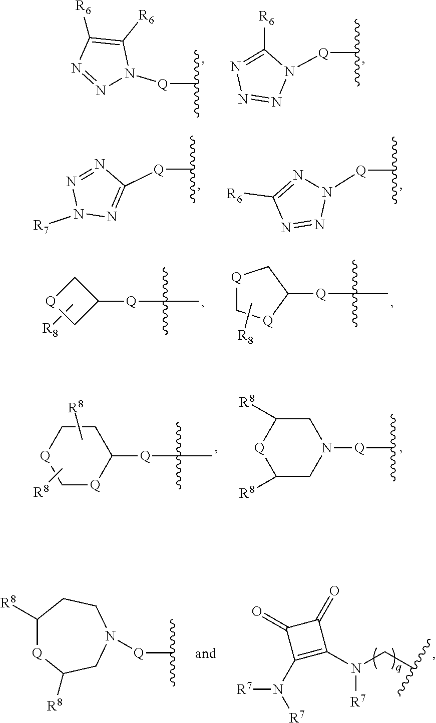

wherein
   each Q is independently absent, —O—, —C(O)—, —C(S)—, —C(O)O—, —(CH₂)_{q}—C(R⁷)₂—, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, thiol, thiolalkyl, or NN(R⁷)₃-alkylene-Q-;
   each R⁸ is independently H, alkyl, hydroxyalkyl, amino, aminoalkyl, alkylamino, thiol, thiolalkyl, heterocyclyl, heteroaryl; or two R⁸ together with the nitrogen atom form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

In some embodiments, the ionizable lipid is a compound in Table I, Table II, Table III, or Table IV.

In some embodiments, the ionizable lipid is Lipid No. 2272 or 2243.

In the CLP or LNMP formulations or the lipid nanoparticle composition, more than one ionizable lipid can be used for the ionizable lipid component: one or more of the ionizable lipids from the compounds of formulas in groups i)-iv) can be used alone or in combination with a different ionizable lipid from the compounds of formulas in groups i)-iv).

In some embodiments, the polynucleotides are polynucleotide constructs, which encode one or more wild type or engineered antigens (or an antibody to an antigen). The antigen may be derived from a tumor, e.g., a tumor specific antigen, a tumor associated antigen, a tumor neoantigen, or a combination thereof. In some embodiments, the polynucleotide construct encodes anti-TNF.

In some embodiments, the antigenic polypeptide encoded by the polynucleotide is a tumor antigenic polypeptide comprising p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12), MAGE-B, MAGE-C, MART-1/Melan-A, MC1R, Myosin/m, MUC1, MUM-1, -2, -3, NA88-A, NF1, NY-ESO-1, NY-BR-1, pl90 minor BCR-abL, Plac-1, Pml/RARa, PRAME, proteinase 3, PSA, PSM, RAGE, RU1 or RU2, SAGE, SART-1 or S ART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.

In some embodiments, the antigenic polypeptide encoded by the polynucleotide is a tumor antigenic polypeptide comprising CD2, CD3, CD4, CD8, CD11b, CD14, CD16, CD19, CD20, CD22, CD25, CD27, CD33, CD37, CD38, CD40, CD44, CD45, CD47, CD52, CD56, CD70, CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbBI, ErbB2, ErbB3, ErbB4, EGFL7, EpCAM, EphA2, EphA3, EphB2, FAP, Fibronectin, Folate Receptor, Foxp3, Ganglioside GM3, GD2, glucocorticoid-induced tumor necrosis factor receptor (GITR), gplOO, gpA33, GPNMB, HLA, HLA-DR, ICOS, IGF1R, Integrin av, Integrin αvβ, LAG-3, Lewis Y, Mesothelin, c-MET, MN Carbonic anhydrase IX, MUC1, MUC16, Nectin-4, KGD2, NOTCH, OX40, OX40L, PD-1, anti-PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof.

In some embodiments, the tumor antigenic polypeptide is IL2 peptide, IL-2-Ra, Anti-CD19 antibody, anti-CD20 antibody, chimeric antigen receptor T cell (CAR-T) antibody, anti-HER2 antibody, etanercept (e.g., Enbrel), adalimumab (e.g., Humira), epoetin alfa (e.g., Epogen), filgrastim (e.g., Neupogen), pembrolizumab (e.g., Keytruda), rituximab (e.g., Rituxan), romiplostim (e.g., Nplate), sargramostim (e.g., Leukine), or a fragment or subunit thereof. In one embodiment, the tumor antigenic polypeptide is IL2 peptide, or a fragment or subunit thereof. In one embodiment, tumor antigenic polypeptide is epoetin alfa (e.g., Epogen), or a fragment or subunit thereof.

In some embodiments, the tumor antigenic polypeptide comprises a tumor antigen selected from the group consisting of a carcinoma, a sarcoma, a melanoma, a lymphoma, a leukemia, and a combination thereof. In one embodiment, the tumor antigenic polypeptide comprises a lung cancer antigen.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA. In one embodiment, the polynucleotide is a circRNA.

In one embodiment, the polynucleotide is an mRNA which encodes a molecule comprising an amino acid sequence having at least 85%, 90%, 95%, 96%, 97%, 98%, 99% or 100% identity to the amino acid sequence provided in any one of Table 3.

In one embodiment, the polynucleotide is an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables V-VII and Table 3.

In some embodiments, the mRNA is (a) a DNA molecule, or (b) an RNA molecule. In the mRNA, T is optionally substituted with U.

In some embodiments, the mRNA is a DNA molecule. The DNA molecule can further comprise a promoter. In some embodiments, the promoter is a T7 promoter, a T3 promoter, or an SP6 promoter. In some embodiments, the promoter is located at the 5′ UTR.

In some embodiments, the mRNA is an RNA molecule. The RNA molecule may be a self-replicating RNA molecule.

In some embodiments, the mRNA is an RNA molecule. The RNA molecule may further comprise a 5′ cap. The 5′ cap can have a Cap 1 structure, a Cap 1 (m6A) structure, a Cap 2 structure, a Cap 3 structure, a Cap 0 structure, or any combination thereof.

In some embodiments, the polynucleotide is an mRNA which encodes an IL-2 molecule. In one embodiment, the IL-2 molecule comprises a naturally occurring IL-2 molecule, a fragment of a naturally occurring IL-2 molecule, or a variant thereof. In one embodiment, the IL-2 molecule comprises a variant of a naturally occurring IL-2 molecule (e.g., an IL-2 variant, e.g., as described herein), or a fragment thereof.

In some embodiments, the mRNA comprises a 5′ untranslated region (UTR) and/or a 3′ UTR.

In some embodiments, the mRNA comprises a 5′ UTR. The 5′ UTR may comprise a Kozak sequence.

In some embodiments, the mRNA comprises a 3′ UTR. In some embodiments, the 3′ UTR comprises one or more sequences derived from an amino-terminal enhancer of split (AES). In some embodiments, the 3′ UTR comprises a sequence derived from mitochondrially encoded 12S mRNA (mtRNRI).

In some embodiments, the mRNA comprises a poly(A) sequence. In one embodiment, the poly(A) sequence is a 110-nucleotide sequence consisting of a sequence of 30 adenosine residues, a 10-nucleotide linker sequence, and a sequence of 70 adenosine residues.

In some embodiments, the natural lipids of the LNMPs are extracted from a plant source, such as lemon or algae. In some embodiments, the natural lipids are extracted from lemon. In some embodiments, the natural lipids are extracted from a bacteria source, such as E. coli or Salmonella typhimurium.

In some embodiments, for the ionizable lipid component, the ionizable lipids from the compounds of formulas in groups i)-iv) can be used in combination with one or more other ionizable lipids. For instance, one or more other ionizable lipids can include 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315. In one embodiment, the additional ionizable lipid included is C12-200.

In some embodiments, the LNMP is produced by a method comprising lipid extrusion. In some embodiments, the LNMP is produced by a method comprising processing a solution comprising a lipid extract of the PMPs in a microfluidics device comprising an aqueous phase, thereby producing the LNMPs. In some embodiments, the aqueous phase comprises the polynucleotides. In some embodiments, the reconstitution is performed in the presence of a sterol, thereby producing a LNMP that comprises natural lipids, an ionizable lipid, and a sterol. In some embodiments, the sterol is cholesterol or sitosterol.

In some embodiments, the reconstitution is performed in the presence of a PEGylated lipid (or a PEG-lipid conjugate), thereby producing a LNMP that comprises natural lipids, the ionizable lipid, and a PEG-lipid conjugate. In some embodiments, the LNMPs further comprise a sterol and a polyethylene glycol (PEG)-lipid conjugate.

In some embodiments, the LNP composition comprises one or more ionizable lipids, one or more synthetic structural lipids, a sterol, and one or more PEG-modified lipids.

In some embodiments, the LNMPs or the LNP composition further comprise a sterol and a polyethylene glycol (PEG)-lipid conjugate.

In some embodiments, the PEG-lipid conjugate is C14-PEG2k, C18-PEG2k, or DMPE-PEG2k. In some embodiments, the PEG-lipid conjugate is PEG-DMG or PEG-PE. In some embodiments, the PEG-DMG is PEG2000-DMG or PEG2000-PE.

In some embodiments, the synthetic structural lipid of the LNP composition is a phospholipid selected from the group consisting of lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof.

In some embodiments, the LNP composition comprises:
about 20 mol % to about 50 mol % of the ionizable lipid,
about 5 mol % to about 60 mol % of the synthetic structural lipids,
about 7 mol % to about 50 mol % of the sterol, and
about 0.5 mol % to about 3 mol % of the polyethylene glycol (PEG)-lipid conjugate.

In some embodiments, the LNP composition comprises:
about 20 mol % to about 50 mol % of the ionizable lipid from Table I, Table II, Table III, or Table IV,
about 5 mol % to about 60 mol % of the synthetic structural lipids,
about 7 mol % to about 50 mol % of the sterol, and
about 0.5 mol % to about 3 mol % of the polyethylene glycol (PEG)-lipid conjugate.

In some embodiments, the LNP composition comprises ionizable lipid:synthetic structural lipids:sterol:PEG-lipid at a molar ratio of about 35:50:12.5:2.5, about 35:20:42.5:2.5, about 35:30:32.5:2.5, about 35:16:46.5:2.5, about 35:25:37.5:2.5, about 35:40:22.5:2.5, about 45:10:43.5:1.5, about 50:20:28.5:1.5, or about 50:10:38.5:1.5.

In some embodiments, the LNMP comprises:
about 20 mol % to about 50 mol % of the ionizable lipid,
about 5 mol % to about 60 mol % of the natural lipids, and optionally a neutral lipid,
about 7 mol % to about 50 mol % of the sterol, and
about 0.5 mol % to about 3 mol % of the polyethylene glycol (PEG)-lipid conjugate.

In one embodiment, the LNMPs comprise the ionizable lipid:natural lipids:sterol:PEG-lipid at a molar ratio of about 35:50:12.5:2.5. In one embodiment, the LNMPs comprise the ionizable lipid:natural lipids:sterol:PEG-lipid at a molar ratio of about 35:20:42.5:2.5. In one embodiment, the LNMPs comprise the ionizable lipid:natural lipids:sterol:PEG-lipid at a molar ratio of about 35:30:32.5:2.5. In one embodiment, the LNMPs comprise the ionizable lipid:natural lipids:sterol:PEG-lipid at a molar ratio of about 35:16:46.5:2.5. In one embodiment, the LNMPs comprise the ionizable lipid:natural lipids:sterol:PEG-lipid at a molar ratio of about 35:25:37.5:2.5. In one embodiment, the LNMPs comprise the ionizable lipid:natural lipids:sterol:PEG-lipid at a molar ratio of about 35:40:22.5:2.5. In one embodiment, the LNMPs comprise the ionizable lipid:natural lipids:sterol:PEG-lipid at a molar ratio of about 45:10:43.5:1.5. In one embodiment, the LNMPs comprise the ionizable lipid:natural lipids:sterol:PEG-lipid at a molar ratio of about 35:16:46.5:2.5. In one embodiment, the LNMPs comprise the ionizable lipid:natural lipids:sterol:PEG-lipid at a molar ratio of about 50:20:28.5:1.5. In one embodiment, the LNMPs comprise the ionizable lipid:natural lipids:sterol:PEG-lipid at a molar ratio of about 50:10:38.5:1.5.

In some embodiments, the LNMP may further comprise a neutral lipid as a helper lipid. In some embodiments, the natural lipids may be used in combination with a neutral lipid as a structural lipid component. The neutral lipid may be used in a molar ratio of neutral lipid:natural lipid of 10:1 to 1:10, or 3:1 to 1:3, e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. Non-limiting examples of neutral lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. In one embodiment, the structural lipid component in the LNMP may comprise a natural lipid+DOPE, or a natural lipid+DSPC.

In these embodiments, the LNMP thus comprises the ionizable lipids described herein, a structural lipid comprising natural lipids and a neutral lipid, a sterol and/or a PEG-lipid.

In some embodiments, the LNMP comprises:
about 20 mol % to about 50 mol % of the ionizable lipid,
about 5 mol % to about 60 mol % of a structural lipid component (i.e., the natural lipids and the neutral lipid),
about 7 mol % to about 50 mol % of the sterol, and
about 0.5 mol % to about 3 mol % of the polyethylene glycol (PEG)-lipid conjugate.

In one embodiment, the LNMPs comprise the ionizable lipid:(natural lipids+ neutral lipid):sterol:PEG-lipid at a molar ratio of about 35:50:12.5:2.5. In one embodiment, the LNMPs comprise the ionizable lipid:(natural lipids+ neutral lipid):sterol:PEG-lipid at a molar ratio of about 35:20:42.5:2.5. For instance, the LNMPs may comprise the ionizable lipid:(natural lipids+ neutral lipid):sterol:PEG-lipid at a molar ratio of about 35:(10+10):42.5:2.5. In one embodiment, the LNMPs comprise ionizable lipid:(natural lipids+ neutral lipid):sterol:PEG-lipid at a molar ratio of about 50:20:28.5:1.5. For instance, the LNMPs may comprise the ionizable lipid:(natural lipids+ neutral lipid):sterol:PEG-lipid at a molar ratio of about 50:(10+10):28.5:1.5.

In some embodiments, the LNMPs comprise:
natural lipids extracted from lemon or algae, and optionally a neutral lipid,
the ionizable lipid from Table I, Table II, Table III, or Table IV,
cholesterol, and
DMG-PEG.

In some embodiments, the LNMPs comprise:
natural lipids extracted from lemon or algae, and optionally a neutral lipid,
the ionizable lipid from Table I, Table II, Table III, or Table IV,
cholesterol, and
DMPE-PEG2k.

In one embodiment, the LNMPs comprise:
natural lipids extracted from lemon, and optionally a neutral lipid,
the ionizable lipid from Table I, Table II, Table III, or Table IV,
cholesterol, and
DMG-PEG or DMPE-PEG2k. The LNMPs may comprise the ionizable lipid:lemon lipids:cholesterol:DMPE-PEG2k at a molar ratio of about 35:50:12.5:2.5, about 35:20:42.5:2.5, or about 50:20:28.5:1.5. The LNMPs may comprise the ionizable lipid:(lemon lipids+ neutral lipid):cholesterol:DMPE-PEG2k at a molar ratio of about 35:50:12.5:2.5, about 35:20:42.5:2.5, or about 50:20:28.5:1.5. The LNMPs may comprise the ionizable lipid:lemon lipids:cholesterol:DMG-PEG at a molar ratio of about 35:50:12.5:2.5, about 35:20:42.5:2.5, or about 50:20:28.5:1.5. The LNMPs may comprise the ionizable lipid:(lemon lipids+ neutral lipid):cholesterol:DMG-PEG at a molar ratio of about 35:50:12.5:2.5, about 35:20:42.5:2.5, or about 50:20:28.5:1.5.

In one embodiment, the LNMPs comprise:
natural lipids extracted from algae, and optionally a neutral lipid,
the ionizable lipid from Table I, Table II, Table III, or Table IV,
cholesterol, and
DMPE-PEG2k. The LNMPs may comprise the ionizable lipid:algae lipids:cholesterol:DMPE-PEG2k at a molar ratio of about 35:20:42.5:2.5, about 35:20:42.5:2.5, or about 50:20:28.5:1.5. The LNMPs may comprise the ionizable lipid:(algae lipids+ neutral lipid):cholesterol:DMPE-PEG2k at a molar ratio of about 35:20:42.5:2.5, about 35:20:42.5:2.5, or about 50:20:28.5:1.5.

In one embodiment, the LNMPs comprise:
natural lipids extracted from algae, and optionally a neutral lipid,
the ionizable lipid from Table I, Table II, Table III, or Table IV,
cholesterol, and
DMG-PEG. The LNMPs may comprise the ionizable lipid:algae lipids:cholesterol:DMG-PEG at a molar ratio of about 35:20:42.5:2.5, about 35:20:42.5:2.5, or about 50:20:28.5:1.5. The LNMPs may comprise the ionizable lipid:(algae lipids+ neutral lipid):cholesterol:DMG-PEG at a molar ratio of about 35:20:42.5:2.5, about 35:20:42.5:2.5, or about 50:20:28.5:1.5.

In some embodiments, the LNMPs comprise:
natural lipids extracted from E. coli or Salmonella typhimurium, and optionally a neutral lipid,
the ionizable lipid from Table I, Table II, Table III, or Table IV,
cholesterol, and
DMPE-PEG2k.

In one embodiment, the LNMPs comprise:
natural lipids extracted from E. coli, and optionally a neutral lipid,
the ionizable lipid from Table I, Table II, Table III, or Table IV,
cholesterol, and
DMPE-PEG2k. The LNMPs may comprise the ionizable lipid: E. coli lipids:cholesterol:DMPE-PEG2k at a molar ratio of about 35:50:12.5:2.5, about 35:20:42.5:2.5, or about 50:20:28.5:1.5. The LNMPs may comprise the ionizable lipid:(E. coli lipids+ neutral lipid):cholesterol:DMPE-PEG2k at a molar ratio of about 35:50:12.5:2.5, about 35:20:42.5:2.5, or about 50:20:28.5:1.5.

In one embodiment, the LNMPs comprise:
natural lipids extracted from Salmonella typhimurium, and optionally a neutral lipid,
the ionizable lipid from Table I, Table II, Table III, or Table IV,
cholesterol, and
DMPE-PEG2k. The LNMPs may comprise the ionizable lipid: Salmonella typhimurium lipids:cholesterol:DMPE-PEG2k at a molar ratio of about 35:20:42.5:2.5, about 35:20:42.5:2.5, or about 50:20:28.5:1.5. The LNMPs may comprise the ionizable lipid:(Salmonella typhimurium lipids+ neutral lipid):cholesterol:DMPE-PEG2k at a molar ratio of about 35:20:42.5:2.5, about 35:20:42.5:2.5, or about 50:20:28.5:1.5.

In some embodiments, the LNMPs comprise:
natural lipids extracted from E. coli or Salmonella typhimurium, and optionally a neutral lipid,
the ionizable lipid from Table I, Table II, Table III, or Table IV,
cholesterol, and
DMG-PEG.

In one embodiment, the LNMPs comprise:
natural lipids extracted from E. coli, and optionally a neutral lipid,
the ionizable lipid from Table I, Table II, Table III, or Table IV,
cholesterol, and
DMG-PEG. The LNMPs may comprise the ionizable lipid: E. coli lipids:cholesterol:DMG-PEG at a molar ratio of about 35:50:12.5:2.5, about 35:20:42.5:2.5, or about 50:20:28.5:1.5. The LNMPs may comprise the ionizable lipid:(E. coli lipids+ neutral lipid):cholesterol:DMG-PEG at a molar ratio of about 35:50:12.5:2.5, about 35:20:42.5:2.5, or about 50:20:28.5:1.5.

In one embodiment, the LNMPs comprise:
natural lipids extracted from Salmonella typhimurium, and optionally a neutral lipid,
the ionizable lipid from Table I, Table II, Table III, or Table IV,
cholesterol, and
DMG-PEG. The LNMPs may comprise the ionizable lipid: Salmonella typhimurium lipids:cholesterol:DMG-PEG at a molar ratio of about 35:20:42.5:2.5, about 35:20:42.5:2.5, or about 50:20:28.5:1.5. The LNMPs may comprise the ionizable lipid:(Salmonella typhimurium lipids+ neutral lipid):cholesterol:DMG-PEG at a molar ratio of about 35:20:42.5:2.5, about 35:20:42.5:2.5, or about 50:20:28.5:1.5.

In some embodiments, the LNMP is a lipophilic moiety selected from the group consisting of a lipoplex, a liposome, a lipid nanoparticle, a polymer-based carrier, an exosome, a lamellar body, a micelle, and an emulsion. In one embodiment, the LNMP is a liposome selected from the group consisting of a cationic liposome, a nanoliposome, a proteoliposome, a unilamellar liposome, a multilamellar liposome, a ceramide-containing nanoliposome, and a multivesicular liposome. In one embodiment, the LNMP is a lipid nanoparticle.

In some embodiments, the LNMP has a size of less than about 200 nm. In one embodiment, the LNMP has a size of less than about 150 nm. In one embodiment, the LNMP has a size of less than about 100 nm. In one embodiment, the LNMP has a size of about 80 nm to about 100 nm. In one embodiment, the LNMP has a size of about 55 nm to about 80 nm.

In some embodiments, the LNMP has an N:P ratio of at least 3, for instance, an N:P ratio of 3 to 100, 3 to 50, 3 to 30, 3 to 20, 3 to 15, 3 to 12, 6 to 30, 6 to 20, 6 to 15, or 6 to 12.

In some embodiments, the RNA composition has a total lipid:polynucleotide weight ratio of about 50:1 to about 10:1. In one embodiment, the RNA composition has a total lipid:polynucleotide weight ratio of about 44:1 to about 24:1. In one embodiment, the RNA composition has a total lipid:polynucleotide weight ratio of about 40:1 to about 28:1. In one embodiment, the RNA composition has a total lipid:polynucleotide weight ratio of about 38:1 to about 30:1. In one embodiment, the RNA composition has a total lipid:polynucleotide weight ratio of about 37:1 to about 33:1.

In some embodiments, the RNA composition, e.g., the aqueous phase, further comprises a HEPES or TRIS buffer. The HEPES or TRIS buffer may have a pH of about 7.0 to about 8.5. The HEPES or TRIS buffer can be at a concentration of about 7 mg/mL to about 15 mg/mL. The aqueous phase may further comprise about 2.0 mg/mL to about 4.0 mg/mL of NaCl.

In some embodiments, the RNA composition, e.g., the aqueous phase comprises water, PBS, or a citrate buffer. In one embodiment, the aqueous phase comprises a citrate buffer having a pH of about 3.2.

In some embodiments, the aqueous phase and the lipid solution are mixed at a 3:1 volumetric ratio.

In some embodiments, the RNA composition further comprises one or more cryoprotectants. The one or more cryoprotectants may be sucrose, glycerol, or a combination thereof. In one embodiment, the RNA composition comprises a combination of sucrose at a concentration of about 70 mg/mL to about 110 mg/mL and glycerol at a concentration of about 50 mg/mL to about 70 mg/mL.

In some embodiments, the RNA composition is a lyophilized composition. The lyophilized RNA composition may comprise one or more lyoprotectants. The lyophilized RNA composition may comprise a poloxamer, potassium sorbate, sucrose, or any combination thereof. In one embodiment, the lyophilized RNA composition comprises a poloxamer, e.g., poloxamer 188.

In some embodiments, the RNA composition is a lyophilized composition. In one embodiment, the lyophilized RNA composition comprises about 0.01 to about 1.0% w/w of the polynucleotides. In one embodiment, the lyophilized RNA composition comprises about 1.0 to about 5.0% w/w lipids. In one embodiment, the lyophilized RNA composition comprises about 0.5 to about 2.5% w/w of TRIS buffer. In one embodiment, the lyophilized RNA composition comprises about 0.75 to about 2.75% w/w of NaCl. In one embodiment, the lyophilized RNA composition comprises about 85 to about 95% w/w of a sugar, e.g., sucrose. In one embodiment, the lyophilized RNA composition comprises about 0.01 to about 1.0% w/w of a poloxamer, e.g., poloxamer 188. In one embodiment, the lyophilized RNA composition comprises about 1.0 to about 5.0% w/w of potassium sorbate.

In another aspect, provided herein is a method of delivering an RNA composition in a subject, comprising administering to the subject the RNA composition discussed in the above aspects of the invention.

In another aspect, provided herein is a method of inducing an immune response in a subject, comprising administering to the subject the RNA composition discussed in the above aspects of the invention.

In another aspect, provided herein is a method of treating or preventing a cancer in a subject, comprising administering to the subject the RNA composition discussed in the above aspects of the invention.

In some embodiments, the LNMP is a lipophilic moiety selected from the group consisting of a lipoplex, a liposome, a lipid nanoparticle, a polymer-based carrier, an exosome, a lamellar body, a micelle, and an emulsion. In one embodiment, the LNMP is a liposome selected from the group consisting of a cationic liposome, a nanoliposome, a proteoliposome, a unilamellar liposome, a multilamellar liposome, a ceramide-containing nanoliposome, and a multivesicular liposome. In one embodiment, the LNMP is a lipid nanoparticle.

In some embodiments, the LNMP has a size of less than about 200 nm. In one embodiment, the LNMP has a size of less than about 150 nm. In one embodiment, the LNMP has a size of less than about 100 nm. In one embodiment, the LNMP has a size of about 55 nm to about 80 nm.

In some embodiments, the RNA composition has a total lipid:polynucleotide weight ratio of about 50:1 to about 10:1. In one embodiment, the RNA composition has a total lipid:polynucleotide weight ratio of about 44:1 to about 24:1. In one embodiment, the RNA composition has a total lipid:polynucleotide weight ratio of about 40:1 to about 28:1. In one embodiment, the RNA composition has a total lipid:polynucleotide weight ratio of about 38:1 to about 30:1. In one embodiment, the RNA composition has a total lipid:polynucleotide weight ratio of about 37:1 to about 33:1.

In some embodiments, the methods provided herein comprising a lipid nanoparticle having a bacteria-derived lipid composition, comprising (a) a natural component comprising one or more lipids extracted from a natural source; and (b) an ionizable lipid.

In some embodiments, the natural source is selected from bacteria, animal, insect, archaea, and fungi.

In some embodiments, the methods provided herein comprising a lipid nanoparticle having a bacteria-derived lipid composition, comprising (a) a bacterial component comprising one or more lipids extracted from a bacterial source; and (b) an ionizable lipid.

one or more lipids extracted from a bacterial source; and (b) an ionizable lipid to the target cell.

In some embodiments, the lipid nanoparticle comprises purified bacterial lipid of the bacteria component (a) in the presence of the ionizable lipid (b) to produce the bacteria-derived lipid composition.

In some embodiments, the bacterial source is selected from Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, and Thermoanaerobacterium. In one embodiment, the bacterial source is Escherichia (e.g., E. coli). In one embodiment, the bacterial source is Salmonella (e.g., Salmonella typhimurium).

In some embodiments, the bacterial component comprises isolated bacterial extracellular vesicles.

In some embodiments, the bacterial component is modified by reconstructing a film comprising the bacterial component in the presence of the ionizable lipid.

In some embodiments, the bacterial component is modified by reconstructing a film comprising the purified bacteria lipids of the bacterial component with the ionizable lipid.

In some embodiments, the ionizable lipid has one or more characteristics selected from the group consisting of:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.

The present invention provides LNP composition or LNMPs comprising one or more active agents or therapeutic agents, methods of making the lipid particles, and methods of delivering and/or administering the lipid particles (e.g., for the treatment of a disease or disorder).

In preferred embodiments, the active agent or therapeutic agent is fully encapsulated within the lipid portion of the lipid particle such that the active agent or therapeutic agent in the lipid particle is resistant in aqueous solution to enzymatic degradation, e.g., by a nuclease or protease. In other preferred embodiments, the lipid particles are substantially non-toxic to mammals such as humans.

In one aspect, provided herein is a particle comprising:
(a) a nucleic acid;
(b) an ionizable lipid comprising less than 50 mol % of the total lipid present in the particle;
(c) a composition of natural source lipids comprising from 10 mol % to 90 mol % of the total lipid present in the particle;
(d) a sterol (e.g. cholesterol or its derivative) comprising from and 5 mol % to 40 mol % of the total lipid present in the particle; and
(e) a conjugated lipid (e.g. PEG-lipid) that inhibits aggregation of particles comprising from 0.5 mol % to 3 mol % of the total lipid present in the particle.

In another aspect, provided herein is a particle comprising:
(a) a nucleic acid;
(b) an ionizable lipid comprising about 10 to about 45 mol % of the total lipid present in the particle;
(c) a composition of natural source lipids comprising from about 10 mol % to about 90 mol % of the total lipid present in the particle;
(d) a sterol (e.g. cholesterol or its derivative) comprising from and 10 mol % to about 50 mol % of the total lipid present in the particle; and
(e) a conjugated lipid (e.g. PEG-lipid) that inhibits aggregation of particles comprising from 0 mol % to about 10 mol % of the total lipid present in the particle.

In certain embodiments, the LNP or LNMP particle comprises: (a) a nucleic acid (e.g., mRNA or circRNA); (b) a ionizable lipid comprising from about 20 mol % to about 45 mol % of the total lipid present in the particle; (c) a non-ionizable lipid comprising from about 13 mol % to about 49.5 mol % of the total lipid present in the particle; and (d) a conjugated lipid that inhibits aggregation of particles comprising from about 0.5 mol % to about 2 mol % of the total lipid present in the particle.

In some embodiments, the LNP or LNMP particle comprises: (a) an mRNA or circRNA; (b) a ionizable lipid comprising from about 25 mol % to about 35 mol % of the total lipid present in the particle; (c) cholesterol or a derivative thereof comprising from about 31.5 mol % to about 42.5 mol % of the total lipid present in the particle; and (d) a PEG-lipid conjugate comprising from about 1 mol % to about 2 mol % of the total lipid present in the particle.

In some embodiments, the LNP or LNMP particle comprises: (a) an mRNA or circRNA; (b) a ionizable lipid comprising from about 52 mol % to about 62 mol % of the total lipid present in the particle; (c) a mixture of a phospholipid and cholesterol or a derivative thereof comprising from about 36 mol % to about 47 mol % of the total lipid present in the particle; and (d) a PEG-lipid conjugate comprising from about 1 mol % to about 2 mol % of the total lipid present in the particle.

In some embodiments, provided herein are methods for treating a disease or disorder in a mammalian subject in need thereof, the method comprising administering to the mammalian subject a therapeutically effective amount of a LNP or LNMP described herein orally.

In some embodiments, provided herein is a composition in the form of enterically coated capsules, tablets, caplets, or multiparticulate carriers such as particles, pellets, granules and beads.

In some embodiments, provided herein are a method and delivery system for the administration of a polynucleotide, wherein the drug, a bile salt or bile acid, and at least one surfactant are present in a single dosage form.

In some embodiments, provided herein is a dosage form comprised of an osmotically activated device in which a semipermeable membrane encapsulates a bile salt or bile acid, at least one surfactant as provided herein, and a hydrophilic drug.

In some embodiments, provided herein is a delayed release pharmaceutical dosage form for oral administration of low molecular weight heparin, wherein the dosage form comprises a composition of: (a) a therapeutically effective amount of low molecular weight heparin; (b) a bile salt or bile acid; (c) at least one surfactant selected from the group consisting of hydrophilic surfactants, lipophilic surfactants, and mixtures thereof; and (d) a means for delaying release of the composition from the dosage form following oral administration. In a preferred embodiment, the composition further includes a solubilizer to ensure good solubilization and/or dissolution of one or more components in the composition.

In some embodiments, the dosage form is not limited with respect to size, shape or general configuration, and may comprise, for example, a capsule, a tablet or a caplet, or a plurality of particles, granules, beads, or pellets that may or may not be encapsulated. Furthermore, either the heparin or the bile salt or bile acid may be present as a coating. In addition, the dosage form or components of the dosage form may be enterically coated; for example, a capsule or tablet may be enterically coated, and multiparticulate dosage forms such as drug-containing particles, pellets, granules and beads may be enterically coated as well. The enteric coating will generally comprise a bioerodible, gradually hydrolyzable and/or gradually water-soluble material, suitable for providing a desired delayed release profile.

In some embodiments, any bile salt or acid may be employed, so long as the selected compound is at least partially solubilized or suspended in the composition.

To ensure good solubilization and/or dissolution of the bile salt or acid, and to minimize precipitation thereof, additional formulation-aiding excipients may be incorporated into the aforementioned dosage form. Such excipients include, for example, bufferants, cosolvents, complexing agents, and crystal growth inhibitors. Additionally, processing techniques such as size reduction, co-precipitation, coacervation, lyophilizing, spray drying, eutectic mixing, solid solutioning or other appropriate techniques may be used to make the bile salt or acid more amenable to rapid dissolution. If suspended, the bile salt or acid can be in any of a number of forms, e.g., crystalline, amorphous, nanosized, micronized, or milled.

In some embodiments, a suitable hydrophilic surfactant will generally have an HLB value of at least 10, while suitable lipophilic surfactants will generally have an HLB value of or less than about 10. The co-administration of low molecular weight heparin with a bile salt or acid and at least one surfactant as provided herein substantially enhances the transmembrane absorption of the drug.

While not wishing to be bound by theory, it is proposed that the substantially homogeneous, optically clear aqueous dispersion that results immediately upon contact with an aqueous medium such as gastrointestinal fluid makes the drug immediately available for bioabsorption, i.e. the drug is rapidly and effectively “presented” to a target absorption site within the body. The optically clear aqueous dispersion that is formed is generally characterized as having an absorbance of less than about 0.3 at 400 nm measured at 100× dilution. In another embodiment, a method is provided for administering low molecular weight heparin to a patient, the method comprising administering a therapeutically effective amount of the polynucleotide along with a bile salt or acid and at least one surfactant selected from the group consisting of hydrophilic surfactants, lipophilic surfactants, and mixtures thereof. Typical dosages for orally administered low molecular weight heparin using the dosage forms of the invention are on the order of 700 to 400,000 IU/day, generally in the range of about 2500 to 10,000 IU/day, while typical dosages for orally administered unfractionated heparin are on the order of 2,500 to 800,000 Units/day. Generally, the drug will be given for the treatment or prevention of thrombosis.

Some embodiments, drug delivery systems are provided that comprise an osmotically activated device, i.e., an osmotically activated tablet or capsule, which houses a therapeutically effective amount of a hydrophilic drug, a bile salt or bile acid, and at least one surfactant selected from the group consisting of hydrophilic surfactants, lipophilic surfactants, and mixtures thereof. In this embodiment, the drug-containing composition is encapsulated in a semipermeable membrane or barrier containing a small orifice. As known in the art with respect to so-called “osmotic pump” drug delivery devices, the semipermeable membrane allows passage of water in either direction, but not drug or other components of the drug-containing composition. Therefore, when the device is exposed to aqueous fluids, water will flow into the device due to the osmotic pressure differential between the interior and exterior of the device, and as water flows into the device, the drug-containing formulation in the interior will be “pumped” out through the orifice. The rate of drug release dD/dt, will be equivalent to the inflow rate of water times the drug concentration. In a preferred embodiment, the osmotically activated device is enterically coated with a coating material effective to provide the desired delayed release profile.

In some embodiments, a drug delivery system is provided for oral administration of a polysaccharide drug, the system comprised of a first dosage form and a second dosage form, wherein the first dosage form contains a therapeutically effective amount of the polysaccharide drug, and the second dosage form contains a bile salt or bile acid in combination with at least one surfactant selected from hydrophilic surfactants, lipophilic surfactants, and mixtures thereof, wherein at least one of the dosage forms is a delayed release dosage form, e.g., coated with an enteric coating. The polysaccharide drug may be, for example, glucosamine, a glycosaminoglycan, dextran, xylan, pentasaccharide, polygalacturonic acid, polymannuronic acid, chitin, pharmaceutically acceptable salts, esters or other derivatives thereof, and combinations of any of the foregoing. The dosage forms may be administered simultaneously or sequentially; in the latter case, either the first dosage form may be administered first, followed by administration of the second dosage form, or the second dosage form may be administered first, followed by administration of the first dosage form.

In some embodiments, the RNA composition, e.g., the aqueous phase, further comprises a HEPES or TRIS buffer. The HEPES or TRIS buffer may have a pH of about 7.0 to about 8.5. The HEPES or TRIS buffer can be at a concentration of about 7 mg/mL to about 15 mg/mL. The aqueous phase may further comprise about 2.0 mg/mL to about 4.0 mg/mL of NaCl.

In some embodiments, the RNA composition, e.g., the aqueous phase comprises water, PBS, or a citrate buffer. In one embodiment, the aqueous phase comprises a citrate buffer having a pH of about 3.2.

In some embodiments, the aqueous phase and the lipid solution are mixed at a 3:1 volumetric ratio.

In some embodiments, the RNA composition further comprises one or more cryoprotectants. The one or more cryoprotectants may be sucrose, glycerol, or a combination thereof. In one embodiment, the RNA composition comprises a combination of sucrose at a concentration of about 70 mg/mL to about 110 mg/mL and glycerol at a concentration of about 50 mg/mL to about 70 mg/mL.

In some embodiments, the RNA composition is a lyophilized composition. The lyophilized mRNA composition may comprise one or more lyoprotectants. The lyophilized RNA composition may comprise a poloxamer, potassium sorbate, sucrose, or any combination thereof. In one embodiment, the lyophilized RNA composition comprises a poloxamer, e.g., poloxamer 188.

In some embodiments, the RNA composition is a lyophilized composition. In one embodiment, the lyophilized RNA composition comprises about 0.01 to about 1.0% w/w of the polynucleotides. In one embodiment, the lyophilized RNA composition comprises about 1.0 to about 5.0% w/w lipids. In one embodiment, the lyophilized RNA composition comprises about 0.5 to about 2.5% w/w of TRIS buffer. In one embodiment, the lyophilized RNA composition comprises about 0.75 to about 2.75% w/w of NaCl. In one embodiment, the lyophilized RNA composition comprises about 85 to about 95% w/w of a sugar, e.g., sucrose. In one embodiment, the lyophilized RNA composition comprises about 0.01 to about 1.0% w/w of a poloxamer, e.g., poloxamer 188. In one embodiment, the lyophilized RNA composition comprises about 1.0 to about 5.0% w/w of potassium sorbate.

In another aspect, provided herein is a method of delivering an RNA in a subject, comprising administering to a subject the RNA composition discussed in the above aspects of the invention.

In another aspect, provided herein is a method of inducing an immune response in a subject, comprising administering to a subject the RNA composition discussed in the above aspects of the invention.

In another aspect, provided herein is a method of treating or preventing a cancer in a subject, comprising administering to the subject the RNA composition discussed in the above aspects of the invention.

In these aspects of the invention, the RNA composition may be administered by oral, intravenous, intradermal, intramuscular, intranasal, intraocular, or rectal, and/or subcutaneous administration. In certain embodiments, the RNA composition is administered by oral, enteral, intravenous, intramuscular, and/or subcutaneous administration.

In some embodiments, the RNA composition is administered at a dosage level sufficient to deliver about 0.01 mg/kg to about 0.2 mg/kg of the RNA to the subject. In some embodiments, the RNA composition is administered at a dosage level sufficient to deliver 0.01 mg/kg, 0.05 mg/kg, 0.1 mg/kg of the RNA to the subject.

In some embodiments, the RNA composition is administered to the subject once, twice, three times, four times, or more. In some embodiments, the RNA composition is administered to the subject once or twice.

In some embodiments, the method further comprises administering an additional therapeutic agent to the subject.

In some embodiments, the additional therapeutic agent is an anti-cancer therapeutic agent.

In some embodiments, the additional therapeutic agent is a therapeutic agent that treats and/or prevents chronic pain. In one embodiment, the additional therapeutic agent is an opioid analgesics such as buprenorphine, a non-steroidal anti-inflammatory drugs (NSAIDs) such as meloxicam SR, or combinations thereof.

In some embodiments, the additional therapeutic agent is administered prior to, concurrent with, or after the administration of the RNA composition.

### Definitions

As used herein, the term “effective amount,” “effective concentration,” or “concentration effective to” refers to an amount of a LNMP, or nucleic acid composition, sufficient to affect the recited result or to reach a target level (e.g., a predetermined or threshold level) in or on a target organism.

As used herein, the term “therapeutic agent” refers to an agent that can act on an animal, e.g., a mammal (e.g., a human), an animal pathogen, or a pathogen vector, such as an antifungal agent, an antibacterial agent, a virucidal agent, an anti-viral agent, an insecticidal agent, a nematicidal agent, an antiparasitic agent, or an insect repellent. As defined herein, the term “nucleic acid” and “polynucleotide” are interchangeable and refer to RNA or DNA that is linear or branched, single or double stranded, or a hybrid thereof, regardless of length (e.g., at least 2, 3, 4, 5, 6, 7, 8, 9, 10, 20, 30, 40, 50, 100, 150, 200, 250, 500, 1000, or more nucleic acids). The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, the terms “circRNA,” “circular polyribonucleotide,” “circular RNA,” and “circular polyribonucleotide molecule” are used interchangeably and mean a polyribonucleotide molecule that has a structure having no free ends (i.e., no free 3′ and/or 5′ ends), for example a polyribonucleotide molecule that forms a circular or end-less structure through covalent (e.g., covalently closed) or non-covalent bonds. The circular polyribonucleotide may be, e.g., a covalently closed polyribonucleotide.

As used herein, the term “expression sequence” is a nucleic acid sequence that encodes a product, e.g., a polypeptide or a regulatory nucleic acid. An exemplary expression sequence that codes for a polypeptide can comprise a plurality of nucleotide triads, each of which can code for an amino acid and is termed as a “codon”.

As used herein, the terms “linear RNA,” “linear polyribonucleotide,” and “linear polyribonucleotide molecule” are used interchangeably and mean a monoribonucleotide molecule or polyribonucleotide molecule having a 5′ and 3′ end. One or both of the 5′ and 3′ ends may be free ends or joined to another moiety. In some embodiments, the linear RNA has a 5′ end or 3′ end that is modified or protected from degradation (e.g., by a 5′ end protectant or a 3′ end protectant). In some embodiments, the linear RNA has non-covalently linked 5′ or 3′ ends. A linear RNA can be used as a starting material for circularization through, for example, splint ligation, or chemical, enzymatic, ribozyme- or splicing-catalyzed circularization methods.

As used herein, the term “polyribonucleotide cargo” herein includes any sequence including at least one polyribonucleotide. In embodiments, the polyribonucleotide cargo includes one or multiple expression sequences, wherein each expression sequence encodes a polypeptide. In embodiments, the polyribonucleotide cargo includes one or multiple noncoding sequences, such as a polyribonucleotide having regulatory or catalytic functions. In embodiments, the polyribonucleotide cargo includes a combination of expression and noncoding sequences. In embodiments, the polyribonucleotide cargo includes one or more polyribonucleotide sequence described herein, such as one or multiple regulatory elements, internal ribosomal entry site (IRES) elements, or spacer sequences.

As used herein, the elements of a nucleic acid are “operably connected” or “operably linked” if they are positioned on the vector such that they can be transcribed to form a linear RNA that can then be circularized into a circular RNA using the methods provided herein.

As used herein, a “spacer” or “spacer sequence” refers to any contiguous, non-coding nucleotide sequence (e.g., of one or more nucleotides) that provides distance or flexibility between two adjacent polynucleotide regions. Exemplary spacer sequences include, but are not limited to, poly(X) sequences as described herein, repetitive or random non-coding DNA or RNA sequences located 3′ or 5′ to open reading frames, or 3′ or 5′ untranslated regions. Any spacer sequence deemed appropriate by the skilled artisan for the polyribonucleotides described herein are contemplated by this disclosure.

As used interchangeably herein, the terms “poly(X)” and “poly(X) sequence” refer to an untranslated, contiguous region of any nucleic acid molecule of at least 5 nucleotides in length and consisting of individual adenine (A), thymine (T), cytosine (C), guanine (G), or uracil (U) residues, or some combination thereof. For example, in some embodiments, a poly(A) sequence may be sequence of adenine residues. In other embodiments, a poly(A-T) sequence is a combination of adenine and thymine residues. In other embodiments, a poly(A-U) sequence may be a combination of adenine and uracil residues. In some embodiments, a poly(A-G) sequence is a combination of adenine and guanine residues. In some embodiments, a poly(G-C) sequence is a combination of guanine and cytosine residues. In some embodiments, a poly(X) sequence may be at least about 50 nucleotides to about 700 nucleotides in length, at least about 60 nucleotides to about 600 nucleotides in length, at least about 70 nucleotides to about 500 nucleotides in length, at least about 80 nucleotides to about 400 nucleotides in length, at least about 90 nucleotides to about 300 nucleotides in length, at least about 100 nucleotides to about 200 nucleotides in length. In some embodiments, the poly(X) sequence may be at least about 50, at least about 100, at least about 200, at least about 300, at least about 400, at least about 500, at least about 600, or at least about 700 nucleotides in length. In some embodiments, a poly(X) sequence may be located 3′ to (e.g., downstream of) an open reading frame (e.g., an open reading frame encoding a polypeptide), and the poly(X) sequence may be 3′ to a termination element (e.g., a stop codon) such that the poly(X) sequence is not translated. In some embodiments, a poly(X) sequence may be located 3′ to a termination element and a 3′ spacer sequence.

As used herein, the terms “nicked RNA,” “nicked linear polyribonucleotide,” and “nicked linear polyribonucleotide molecule” are used interchangeably and mean a polyribonucleotide molecule having a 5′ and 3′ end that results from nicking or degradation of a circular RNA.

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, 150, 200, 250, 300, 350, 400, 450, 500, 550, 600, 650, 700, 750, 800, 850, 900, 950, 1000, or more than 1000 amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics. The polypeptide may be, e.g., at least 0.1, at least 1, at least 5, at least 10, at least 15, at least 20, at least 30, at least 40, at least 50, or more than 50 kD in size. The polypeptide may be a full-length protein. Alternatively, the polypeptide may comprise one or more domains of a protein.

As used herein, the term “animal” refers to humans and non-human animals (including for example, dogs, cats, horses, rabbits, zoo animals, cows, pigs, sheep, chickens, and non-human primates).

As used herein, the term “infection” refers to the presence or colonization of a pathogen in an animal (e.g., in one or more parts of the animal), on an animal (e.g., on one or more parts of the animal), or in the habitat surrounding an animal, particularly where the infection decreases the fitness of the animal, e.g., by causing a disease, disease symptoms, or an immune (e.g., inflammatory) response.

As used herein the term “pathogen” refers to an organism, such as a microorganism or an invertebrate, which causes disease or disease symptoms in an animal by, e.g., (i) directly infecting the animal, (ii) producing agents that causes disease or disease symptoms in an animal (e.g., bacteria that produce pathogenic toxins and the like), and/or (iii) by eliciting an immune (e.g., inflammatory response) in animals (e.g., biting insects, e.g., bedbugs). As used herein, pathogens include, but are not limited to, bacteria, protozoa, parasites, fungi, nematodes, insects, viroids and viruses, or any combination thereof, wherein each pathogen is capable, either by itself or in concert with another pathogen, of eliciting disease or symptoms in humans.

As used herein, the term “antibody” encompasses an immunoglobulin, whether natural or partly or wholly synthetically produced, and fragments thereof, capable of specifically binding to an antigen. The term also covers any protein having a binding domain which is homologous to an immunoglobulin binding domain. These proteins can be derived from natural sources, or partly or wholly synthetically produced. “Antibody” further includes a polypeptide comprising a framework region from an immunoglobulin gene or fragments thereof that specifically binds and recognizes an antigen. Use of the term “antibody” is meant to include whole antibodies; polyclonal, monoclonal and recombinant antibodies; fragments thereof; and further includes single-chain antibodies (nanobodies); humanized antibodies; murine antibodies; chimeric, mouse-human, mouse-primate, primate-human monoclonal antibodies; anti-idiotype antibodies; antibody fragments, such as, e.g., scFv, (scFv)2, Fab, Fab′, and F(ab′)2, F(ab1)2, Fv, dAb, and Fd fragments; diabodies; and antibody-related polypeptides. “Antibody” further includes bispecific antibodies and multispecific antibodies.

As used herein, the term “heterologous” refers to an agent (e.g., a polypeptide) that is either (1) exogenous to the plant (e.g., originating from a source that is not the plant or plant part from which the PMP is produced) (e.g., an agent which is added to the PMP using loading approaches described herein) or (2) endogenous to the plant cell or tissue from which the PMP is produced, but present in the PMP (e.g., added to the PMP using loading approaches described herein, genetic engineering, as well as in vitro or in vivo approaches) at a concentration that is higher than that found in nature (e.g., higher than a concentration found in a naturally-occurring plant extracellular vesicle).

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “modified NMPs” or “modified LNMPs” refers to a composition including a plurality of NMPs or LNMPs that include one or more heterologous agents (e.g., one or more exogenous lipids, such as a ionizable lipids, e.g., a NMP or LNMP comprising an ionizable lipid and a sterol and/or a PEGylated lipid) capable of increasing cell uptake (e.g., animal cell uptake, plant cell uptake, bacterial cell uptake, or fungal cell uptake) of the NMP or LNMP, or a portion or component thereof, relative to an unmodified NMP or LNMP; capable of enabling or increasing delivery of a heterologous functional agent (e.g., an agricultural or therapeutic agent) by the NMP or LNMP to a cell, and/or capable of enabling or increasing loading (e.g., loading efficiency or loading capacity) of a heterologous functional agent (e.g., an agricultural or therapeutic agent). The NMPs or LNMPs may be modified in vitro or in vivo.

As used herein, the term “unmodified NMPs” or “unmodified LNMPs” refers to a composition including a plurality of NMPs or LNMPs that lack a heterologous cell uptake agent capable of increasing cell uptake (e.g., animal cell uptake, plant cell uptake, bacterial cell uptake, or fungal cell uptake) of the NMP.

As used herein, the term “modified PMPs” or “modified LPMPs” refers to a composition including a plurality of PMPs or LPMPs that include one or more heterologous agents (e.g., one or more exogenous lipids, such as a ionizable lipids, e.g., a PMP or LPMP comprising an ionizable lipid and a sterol and/or a PEGylated lipid) capable of increasing cell uptake (e.g., animal cell uptake, plant cell uptake, bacterial cell uptake, or fungal cell uptake) of the PMP or LPMP, or a portion or component thereof, relative to an unmodified PMP or LPMP; capable of enabling or increasing delivery of a heterologous functional agent (e.g., an agricultural or therapeutic agent) by the PMP or LPMP to a cell, and/or capable of enabling or increasing loading (e.g., loading efficiency or loading capacity) of a heterologous functional agent (e.g., an agricultural or therapeutic agent). The PMPs or LPMPs may be modified in vitro or in vivo.

As used herein, the term “unmodified PMPs” or “unmodified LPMPs” refers to a composition including a plurality of PMPs or LPMPs that lack a heterologous cell uptake agent capable of increasing cell uptake (e.g., animal cell uptake, plant cell uptake, bacterial cell uptake, or fungal cell uptake) of the PMP.

As used herein, the term “cell uptake” refers to uptake of a NMP or LNMP or a portion or component thereof (e.g., a polynucleotide carried by the NMP or LNMP) by a cell, such as an animal cell, a plant cell, bacterial cell, or fungal cell. For example, uptake can involve transfer of the NMP (e.g., LNMP) or a portion of component thereof from the extracellular environment into or across the cell membrane, the cell wall, the extracellular matrix, or into the intracellular environment of the cell). Cell uptake of NMPs (e.g., LNMPs) may occur via active or passive cellular mechanisms. Cell uptake includes aspects in which the entire NMP (e.g., LNMP) is taken up by a cell, e.g., taken up by endocytosis. In some embodiments, one or more polynucleotides are exposed to the cytoplasm of the target cell following endocytosis and endosomal escape. In some embodiments, a modified LNMP (e.g., a LNMP comprising an ionizable lipid, e.g., a LNMP comprising an ionizable lipid and a sterol and/or a PEGylated lipid) has an increased rate of endosomal escape relative to an unmodified LNMP. Cell uptake also includes aspects in which the NMP (e.g., LNMP) fuses with the membrane of the target cell. In some embodiments, one or more polynucleotides are exposed to the cytoplasm of the target cell following membrane fusion. In some embodiments, a LNMPs has an increased rate of fusion with the membrane of the target cell (e.g., is more fusogenic) relative to an unmodified LNMP.

As used herein, the term “cell-penetrating agent” refers to agents that alter properties (e.g., permeability) of the cell wall, extracellular matrix, or cell membrane of a cell (e.g., an animal cell, a plant cell, a bacterial cell, or a fungal cell) in a manner that promotes increased cell uptake relative to a cell that has not been contacted with the agent.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, sap (e.g., xylem sap and phloem sap), and various forms of cells and culture (e.g., single cells, protoplasts, embryos, and callus tissue). The plant tissue may be in a plant or in a plant organ, tissue, or cell culture. In addition, a plant may be genetically engineered to produce a heterologous protein or RNA.

As used herein, the term “Bacteria” refers to whole bacteria or parts of bacteria. Further divisions of bacteria can be classified as coccals, bacillus, spirillum, or vibrio, and varying phylums include but are not limited to Proteobacteria, Firmicutes, Bacteroids, sphingobacteria, Flavobacteria, Fusobacteria, Spirochaetes, Chlorobia, Cyanobacteria, Thermomicrobia, Xenobacteria, or Aquificae. Example of specific bacteria species include Staphylococcus aureus, Escherichia coli, Salmonella typhimurium, Streptococcus pneumoniae, and Pseudomonas aeruginosa. Parts of bacteria include cellular components such as peptidoglycan, outer membranes, inner membranes, cell walls, RNA polymerase, metabolic products, polypeptides, proteins. Flagella, pili, ribosomes, mesosome, cytoplasm, or chromosome. A bacteria may be genetically engineered to produce a heterologous protein or RNA, or may be genetically engineered to not produce an endogenous protein or RNA.

As used herein, the term “Arthropod” refers to any animal within the phylum Arthropoda, or any animal section, part, organ, tissue, egg, cell, or progeny of the same. Example animals include insects, spiders, and crustaceans. Arthropod cells include, without limitation, cells from eggs, suspension cultures, embryos, tissue, organs, exoskeletons, segments, and appendages. Arthropod parts include body segments, appendages, exoskeleton, eggs, organs, embryos, and various forms of cells and culture. Arthropod tissue may be in an arthropod or in an organ, tissue, or cell culture. An arthropod may be genetically engineered to produce a heterologous protein or RNA. An arthropod may be genetically engineered to not produce an endogenous protein or RNA.

As used herein, the term “Fungi” refers to whole fungi, fungi organs, fungi tissue, spores, fungi cells, and progeny of the same. Example fungi include yeasts, mushrooms, molds, and mildews. Fungi cells include without limitation cells from spores, suspension cultures, mycelium, hyphae, thallus, cell walls, tissue, gametophytes, sporophytes, and organs. Fungal tissue may be in a fungus or in an organ, tissue, or cell culture. A fungus may be genetically engineered to produce a heterologous protein or RNA. A fungus may be genetically engineered to not produce an endogenous protein or RNA.

As used herein, the term “Archaea” refers to whole archaea or parts of archaea. Example archaea include euryarchaeota, crenarchaeota, and koraarchaeota. Parts of archaea include cellular components such as RNA polymerases, glycerol-ether lipids, membranes, cell walls, polypeptides, proteins, and metabolic products. Archaea may be genetically engineered to produce a heterologous protein or RNA, or may be genetically engineered to not produce an endogenous protein or RNA.

As used herein, the term “plant extracellular vesicle”, “plant EV”, or “EV” refers to an enclosed lipid-bilayer structure naturally occurring in a plant. Optionally, the plant EV includes one or more plant EV markers. As used herein, the term “plant EV marker” refers to a component that is naturally associated with a plant, such as a plant protein, a plant nucleic acid, a plant small molecule, a plant lipid, or a combination thereof, including but not limited to any of the plant EV markers listed in the Appendix. In some instances, the plant EV marker is an identifying marker of a plant EV but is not a pesticidal agent. In some instances, the plant EV marker is an identifying marker of a plant EV and also a pesticidal agent (e.g., either associated with or encapsulated by the plurality of PMPs or LPMPs, or not directly associated with or encapsulated by the plurality of PMPs or LPMPs).

As used herein, the term “natural messenger pack” or “NMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a natural source or segment, portion, or extract thereof, including lipid or non-lipid components (e.g., peptides, nucleic acids, or small molecules) associated therewith and that has been enriched, isolated or purified from a natural source, a part of a natural source, or a cell of a natural source, the enrichment or isolation removing one or more contaminants or undesired components from the source. NMPs may be highly purified preparations of naturally occurring EVs. Preferably, at least 1% of contaminants or undesired components from the natural source are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source, e.g., source cell wall components; pectin; organelles (e.g., mitochondria; plastids such as chloroplasts, leucoplasts or amyloplasts; and nuclei); chromatin (e.g., a chromosome); or molecular aggregates (e.g., protein aggregates, protein-nucleic acid aggregates, lipoprotein aggregates, or lipido-proteic structures). Preferably, a NMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the natural source as measured by weight (w/w), spectral imaging (% transmittance), or conductivity (S/m).

As used herein, the term “plant messenger pack” or “PMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell, the enrichment or isolation removing one or more contaminants or undesired components from the source plant. PMPs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, protein-nucleic acid aggregates, lipoprotein aggregates, or lipido-proteic structures). Preferably, a PMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m).

A lipid reconstructed NMP (LNMP) is used herein. For instance, a lipid reconstructed PMP (LPMP) is used herein. The terms “lipid reconstructed NMP” and “LNMP” refer to a NMP that has been derived from a lipid structure (e.g., a lipid bilayer, unilamellar, multilamellar structure; e.g., a vesicular lipid structure) derived from (e.g., enriched, isolated or purified from) a natural source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LNMP, as is described herein. The method may, if desired, further comprise sonication, freeze/thaw treatment, and/or lipid extrusion, e.g., to reduce the size of the reconstituted NMPs. Alternatively, LNMPs may be produced using a microfluidic device (such as a NanoAssemblr® IGNITE™ microfluidic instrument (Precision NanoSystems)). The terms “lipid reconstructed PMP” and “LPMP” are defined in the same manner as “lipid reconstructed NMP” and “LNMP,” when the natural source is a plant source.

As used herein, the term “pure” refers to a PMP preparation in which at least a portion (e.g., at least 20%, 25%, 30%, 40%, 45%, 50%, 55%, 60%, 70%, 80%, 90%, 95%, 96%, 98%, 99%, or 100%) of plant cell wall components, plant organelles (e.g., mitochondria, chloroplasts, and nuclei), or plant molecule aggregates (protein aggregates, protein-nucleic acid aggregates, lipoprotein aggregates, or lipido-proteic structures) have been removed relative to the initial sample isolated from a plant, or part thereof.

As used herein, the term “complex lipid particle” refers to a lipid particle that has a complexity characterized by comprising a wide variety of lipids, including structural lipids extracted from one or more natural sources (such as plants or bacteria), and optionally at least one exogenous ionizable lipid. The complex lipid particle may comprise between 10% w/w and 99% w/w structural lipids derived from a lipid structure from one or more natural sources, e.g., it may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a lipid structure from one or more natural sources. In some instances, a complex lipid particle incorporating natural lipid extracts may also be referred to as a natural messenger pack (NMP). For instance, a complex lipid particle incorporating plant lipid extracts may also be referred to as a plant messenger pack (PMP). In some instances, a complex lipid particle incorporating natural lipid extracts and at least one exogenous ionizable lipid may also be referred to as a lipid reconstructed natural messenger pack (LNMP). For instance, a complex lipid particle incorporating plant lipid extracts and at least one exogenous ionizable lipid may also be referred to as a lipid reconstructed plant messenger pack (LPMP). Thus, any disclosure herein describing the features relating to LNMP and LNMP formulation are applicable to CLP and CLP formulation.

The complex lipid particle may contain 3-1000 lipids extracted from one or more natural (e.g., plant, bacteria) sources. The complex lipid particle may contain natural (e.g., plant, bacteria) lipids from at least 1, at least 2, at least 3, at least 4, at least 5, at least 6, at least 7, at least 8, at least 9, or at least 10 different classes or sub-classes of lipids from the natural (e.g., plant, bacteria) source. The complex lipid particle may comprise all or a fraction of the lipid species present in the lipid structure from the natural (e.g., plant, bacteria) source, e.g., it may contain at least 10%, at least 20%, at least 30%, at least 40%, at least 50%, at least 60%, at least 70%, at least 80%, at least 90%, or virtually 100% of the lipid species present in the lipid structure from the natural source. The complex lipid particle may comprise all or a fraction of the lipid species present in the lipid structure from a particular natural source. For instance, it may contain at least 10%, at least 20%, at least 30%, at least 40%, at least 50%, at least 60%, at least 70%, at least 80%, at least 90%, or virtually 100% of the lipid species present in the lipid structure from a plant source or from a bacteria source.

The complex lipid particle may comprise reduced or minimized protein matter endogenous to the one or more natural (i.e. plant, bacteria) sources, e.g., it may contain 0% w/w, less than 1% w/w, less than 5% w/w, less than 10% w/w, less than 15% w/w, less than 20% w/w, less than 30% w/w, less than 40% w/w, or less than 50% w/w of the protein matter endogenous to the one or more natural (e.g., plant, bacteria) sources. In some instances, the lipid bilayer of the complex lipid particle does not contain proteins.

The complex lipid particle may also include synthetic structural lipids such as neutral lipids as the structural lipid component. The structural lipid component of the complex lipid particle may comprise between 10% w/w and 99% w/w structural lipids derived from a synthetic lipid structure (as opposed to the lipids extracted from a natural source), e.g., it may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a synthetic lipid structure.

The complex lipid particle may further comprise at least two exogenous lipids. The complex lipid particle may include at least 1% w/w, at least 2% w/w, at least 5% w/w, at least 10% w/w, at least 15% w/w, at least 20% w/w, at least 25% w/w, at least 30% w/w, at least 40% w/w, at least 50% w/w, at least 60% w/w, at least 70% w/w, at least 80% w/w, or about 90% w/w exogenous lipids. Exemplary exogenous lipids include sterols and PEG-lipid conjugate. The complex lipid particle may be used to encapsulate one or more exogenous nucleic acids or polynucleotides encoding one or more peptides, polypeptides, or proteins, to enable delivery of the exogenous nucleic acids or polynucleotides to a target cell or tissue.

As used herein, the term “exogenous lipid” refers to a lipid that is exogenous to the natural source (e.g., plant, bacteria), i.e., a lipid originates from a source that is not the natural source from which the lipids are extracted (e.g., a lipid that is added to the complex lipid particle formulation using method described herein). The term “exogenous lipid” does not exclude a natural-derived lipid (such as a plant-derived sterol). That is to say, an exogenous lipid can be a natural-derived lipid (such as a plant-derived sterol that is exogenous to the plant source from which the lipids are extracted, e.g., an exogenous lipid can be a plant derived sterol that is added to the complex lipid particle formulation). As another example, an exogenous lipid can be a natural-derived lipid that is exogenous to the particular natural source from which the lipids are extracted (e.g., a bacteria-derived lipid that is exogenous to the plant source from which the lipids are extracted, or vice versa). An exogenous lipid may be a cell-penetrating agent, may be capable of increasing delivery of one or more polynucleotides by the complex lipid formulation to a cell, and/or may be capable of increasing loading (e.g., loading efficiency or loading capacity) of a polynucleotide. In some embodiments, the exogenous lipid may be a stabilizing lipid. In some embodiments, the exogenous lipid may be a structural lipid (e.g., a synthetic structural lipid). Exemplary exogenous lipids include ionizable lipids, synthetic structural lipids, sterols, and PEGylated lipids.

As used herein, the term “cationic lipid” refers to an amphiphilic molecule (e.g., a lipid or a lipidoid) that is positively charged, containing a cationic group (e.g., a cationic head group).

As used herein, the term “ionizable lipid” refers to an amphiphilic molecule (e.g., a lipid or a lipidoid, e.g., a synthetic lipid or lipidoid) containing a group (e.g., a head group) that can be ionized, e.g., dissociated to produce one or more electrically charged species, under a given condition (e.g., pH).

It has been surprisingly found that ionizable lipids comprising alkyl chains with multiple sites of unsaturation, e.g., at least two or three sites of unsaturation, are particularly useful for forming lipid particles with increased membrane fluidity. A number of ionizable lipids and related analogs, suitable for use herein, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes.

In some embodiments, ionizable lipids are ionizable such that they can dissociate to exist in a positively charged form depending on pH. The ionization of an ionizable lipid affects the surface charge of a lipid nanoparticle comprising the ionizable lipid under different pH conditions. The surface charge of the lipid nanoparticle in turn can influence its plasma protein absorption, blood clearance, and tissue distribution (Semple, S. C., et al., Adv. Drug Deliv Rev 32:3-17 (1998)) as well as its ability to form endosomolytic non-bilayer structures (Hafez, I. M., et al., Gene Ther 8: 1188-1196 (2001)) that can influence the intracellular delivery of nucleic acids.

In some embodiments, ionizable lipids are those that are generally neutral, e.g., at physiological pH (e.g., pH about 7), but can carry net charge(s) at an acidic pH or basic pH. In one embodiment, ionizable lipids are those that are generally neutral at pH about 7, but can carry net charge(s) at an acidic pH. In one embodiment, ionizable lipids are those that are generally neutral at pH about 7, but can carry net charge(s) at a basic pH.

In some embodiments, ionizable lipids do not include those cationic lipids or anionic lipids that generally carry net charge(s) at physiological pH (e.g., pH about 7).

As used herein, the term “lipidoid” refers to a molecule having one or more characteristics of a lipid.

As used herein, the term “stable LNMP formulation” or “stable CLP formulation” refers to a CLP formulation or a LNMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of CLPs or LNMPs (e.g., CLPs or LNMPs per mL of solution) relative to the number of CLPs or LNMPs in the CLP formulation or LNMP formulation (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., cell wall penetrating activity and/or activity of the RNA formulated within the CLP or LNMP) relative to the initial activity of the CLP or LNMP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

Alternatively, the expression refers to a CLP formulation or LNMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity relative to the initial activity of the CLP formulation or LNMP formulation (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

Alternatively, the expression refers to a CLP formulation or a LNMP formulation that over a period of time (e.g., at least 24 hours, at least 48 hours, at least 1 week, at least 2 weeks, at least 3 weeks, at least 4 weeks, at least 30 days, at least 60 days, or at least 90 days) retains their particle size, i.e., the particle size does not increase, or has an increase of no more than 5% (e.g., no more than 5%, 10%, 15%, 20%, 25%, 30%, 35%, 40%, 45%, 50%, 55%, 60%, 65%, 70%, 75%, 80%, 85%, 90%, 95%, 100%, 150%, 2-fold, 2.5-fold, or 3-fold) relative to the initial size of the CLPs or LNMPs (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

In some embodiments, the stable CLP or LNMP formulation continues to encapsulate or remains associated with an exogenous peptide, polypeptide, or protein with which the CLP or LNMP formulation has been loaded, e.g., continues to encapsulate or remains associated with an exogenous peptide, polypeptide, or protein for at least 24 hours, at least 48 hours, at least 1 week, at least 2 weeks, at least 3 weeks, at least 4 weeks, at least 30 days, at least 60 days, at least 90 days, or 90 or more days.

As used herein, the term “treatment” refers to administering a pharmaceutical composition to an animal for prophylactic and/or therapeutic purposes. To “prevent an infection” refers to prophylactic treatment of an animal that does not yet have a disease or condition, but which is susceptible to, or otherwise at risk of, a particular disease or condition. To “treat an infection” refers to administering treatment to an animal already suffering from a disease to improve or stabilize the animal's condition.

### BRIEF DESCRIPTION OF THE DRAWINGS

FIG. 1 is a photograph showing the level of nanoluciferase in mice treated orally (PO) with reconstructed LPMPs (recPMPs) derived from lemon, formulated with C12-200 as the ionizable lipid, and comprising nanoluciferase (nLuc) mRNA-nLuc-Flag and nLuc secreted.

FIG. 2 is a photograph showing the level of nanoluciferase in mice treated orally (PO) with reconstructed LPMPs (recPMPs) derived from lemon, formulated with C12-200 as the ionizable lipid, and comprising nanoluciferase (nLuc) mRNA-nLuc-Flag and nLuc secreted in liver, stomach, colon, spleen, small intestine, mesenteric lymph nodes, pancreas, and caecum with 5 minutes exposure time.

FIG. 3 is a photograph showing the level of nanoluciferase in mice treated orally (PO) with reconstructed LPMPs (recPMPs) derived from lemon, formulated with C12-200 as the ionizable lipid, and comprising nanoluciferase (nLuc) mRNA-nLuc-Flag and nLuc secreted in liver, stomach, colon, spleen, small intestine, mesenteric lymph nodes, pancreas, and caecum with 5 minutes exposure time.

FIG. 4 is an illustration of organs showing the level of nanoluciferase in mice treated orally (PO) with reconstructed LPMPs (recPMPs) derived from lemon, formulated with C12-200 as the ionizable lipid, and comprising nanoluciferase (nLuc) mRNA-nLuc-Flag and nLuc secreted in liver, stomach, colon, spleen, small intestine, mesenteric lymph nodes, pancreas, and caecum.

FIGS. 5A-5F show the average radiance in the liver (FIG. 5A), spleen (FIG. 5B), pancreas (FIG. 5C), MLNs (FIG. 5D), gastro-intestine (GI) (FIG. 5E), and inguinal lymph nodes (FIG. 5F) of mice 24 hours after either oral (PO) or intrajejunal (IJ) delivery of a mRNA-LNP formulation employing ionizable lipid 2243 (nLuc-Flag mRNA, IJ-10 ug, PO-200 ug). N=5/group as compared to naïve mice (n=2).

FIG. 6A shows a representative section of the small intestine (SI) from a naïve tdTomato mouse as a negative control. FIG. 6B shows a representative section of the SI 48-hours post-intrajejunal administration of 2243 a mRNA-LNP formulation employing the ionizable lipid 2243 (nLuc-Flag: CRE mRNA, 15 ug; N=2), wherein the light gray indicates transfected cells. FIG. 6C highlights a Peyer's Patch shown in FIG. 6B. FIG. 6D highlights villi shown in FIG. 6B.

FIG. 7 depicts the concentration of anti-TNFα antibody levels 24 h post-dose in the plasma of the mice given 0% DSS and a single intravenous dose of a LNP/mRNA formulation employing the ionizable lipid 2243 (LNP 2243/anti-TNFα mRNA, 0.6 mg/kg) and in the plasma of the mice given 2% DSS and a single intravenous dose of a LNP/mRNA formulation employing the ionizable lipid 2243 (LNP 2243/anti-TNFα mRNA, 0.6 mg/kg). The control was the plasma from naïve mice (0% DSS and no administration of LNP 2243/anti-TNFα mRNA). N=5/group.

FIG. 8A shows the concentration of TNFα in feces three-days post dose in the mice given 2% DSS and in the mice given 2% DSS and a single intravenous dose of a LNP/mRNA formulation employing the ionizable lipid 2243 (LNP 2243/anti-TNFα mRNA, 0.6 mg/kg). The control was feces from naïve mice (0% DSS and no administration of LNP 2243/anti-TNFα mRNA). N=5/group. FIG. 8B shows the concentration of calprotectin in feces pre-dose (3 days after the start of DSS), 24 h post-dose (5 days after start of DSS) in the mice given 2% DSS and in the mice given 2% DSS and a single intravenous dose of LNP/mRNA formulation employing the ionizable lipid 2243 (2243 LNP/anti-TNFα mRNA, 0.6 mg/kg), and 3 days post-dose (7 days after the start of DSS) in the mice given 2% DSS and in the mice given 2% DSS and a single intravenous dose of LNP/mRNA formulation employing the ionizable lipid 2243 (2243 LNP/anti-TNFα mRNA, 0.6 mg/kg). The control was feces from naïve mice (0% DSS and no administration of LNP 2243/anti-TNFα mRNA). N=5/group.

FIG. 9 shows the measured antibody concentration (huIgG, ng/mL) in the plasma of mice at Days 0, 1, 3, 7, and 14, respectively, after a single intravenous dose of an exemplary LPMP/mRNA formulation employing the ionizable lipid 2272 (recLemon LPMP 2272/anti-PCSK9 mRNA, 0.3 mg/kg) N=5.

FIG. 10A shows the measured antibody concentration (huIgG, ng/mL) in the plasma of mice 24 h after a single intravenous dose of an exemplary LPMP/mRNA formulation the ionizable lipid 2272 (recLemon LPMP 2272/anti-PCSK9 mRNA, 0.3 mg/kg) N=10. PBS was used as a control (N=1). FIG. 10B shows the measured antibody concentration (huIgG, ng/mL) in MLNs and colon/caecum of mice 24 h after a single intravenous dose of an exemplary LPMP/mRNA formulation employing the ionizable lipid 2272 (recLemon LPMP 2272/anti-PCSK9 mRNA, 0.3 mg/kg) N=10. PBS was used as a control (N=1).

FIGS. 11A-11D show the antibody concentration (huIgG, ng/mL) in the colon/caecum contents, mesenteric lymph nodes (MLNs), small intestine (SI) content, and plasma of mice 24 hours after intrajejunal delivery of an exemplary LPMP/mRNA formulation employing the ionizable lipid 2272 (anti-PCSK9 mRNA, 65-75 ug). N=19/3 independent experiments as compared to PBS (n=16/3 independent experiments). Statistically identified outliers were removed.

### DETAILED DESCRIPTION

The inventors have surprising found that when administering a nano-luciferase via oral gavage intracellularly (nluc-Flag) and via secretion (nLuc-secreted), resulting in broad transfection of the digestive tract (extra-hepatic tissues) and lymphoid organs, no activity was observed in the liver. nLuc-Flag and nLuc-secreted were not detected in the liver; they were only detected in the GI tract and associated lymphoid tissues. In addition, a higher expression of nLuc-secreted in the colon was observed, as compared to nluc-Flag.

Featured herein are RNA therapeutics that can safely direct to the lymphatic transport system without delivering to the liver. Such selectivity would be useful for treating pancreatitis, IBD, ulcerative colitis, Crohn's disease, colorectal cancer, and also oral vaccination (norovirus, RSV, flu, shingle, COVID, and the like).

In some embodiments, the potentially unlocking RNA therapies include, but not limited to GI cancers (i.e. stomach, pancreas, colon); mRNA for cancer antigens, diptheria toxin, anti-PD1, chemokines, Cytokines (Il2, 12, 27, IFNg, IL15), receptors, pancreatitis (mRNA for anti-inflammatory drugs), IBD (mRNA for IL10, anti-TNF, siRNA for TNFa, mRNA for inhibitory receptors), ulcerative colitis, Crohn's disease, infectious diseases (viral, bacterial & fungal), and microbiome modulation.

The RNA compositions may be used to induce a balanced immune response against cancers, comprising both cellular and humoral immunity, without risking the possibility of insertional mutagenesis, for example. These RNA compositions include one or more polynucleotides (e.g., RNA such as mRNA or circRNA) encoding one or more tumor antigenic polypeptides, formulated within a complex lipid particle (CLP). In some embodiments, the CLP is a lipid reconstructed natural messenger packs (LNMPs) comprising lipids extracted from one or more natural sources (i.e., natural lipids) and an ionizable lipid. NMPs are lipid assemblies produced wholly or in part from natural source extracellular vesicles (EVs), or segments, portions, or extracts thereof. PMPs are lipid assemblies produced wholly or in part from plant extracellular vesicles (EVs), or segments, portions, or extracts thereof. LNMPs are NMPs derived from a lipid structure wherein the lipid structure is disrupted and reassembled or reconstituted in a liquid phase. LPMPs are PMPs derived from a lipid structure wherein the lipid structure is disrupted and reassembled or reconstituted in a liquid phase.

The disclosure also includes a method for making a RNA composition, comprising reconstituting a film comprising purified NMP lipids in the presence of an ionizable lipid to produce a LNMP comprising the ionizable lipid, and loading into the LNMPs with one or more polynucleotides encoding one or more polypeptides.

### Complex Lipid Particles and Lipid Reconstructed Natural Messenger Packs (LNMPs)

### Complex Lipid Particles

Complex lipid particles (CLPs) described herein comprise a wide variety of lipids, including structural lipids extracted from one or more natural sources (such as plants or bacteria). In some embodiments, a complex lipid particle is a natural messenger pack (NMP) incorporating natural lipid extracts. In some embodiments, a complex lipid particle is a lipid reconstructed natural messenger pack (LNMP) incorporating natural lipid extracts and at least one exogenous ionizable lipid.

The complex lipid particles may also comprise at least exogenous ionizable lipid. The ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.

The complex lipid particle may comprise between 10% w/w and 99% w/w structural lipids derived from a lipid structure from one or more natural sources, e.g., it may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a lipid structure from one or more natural sources.

In some embodiments, the complex lipid particle comprises about 10-95% w/w of the natural (e.g., plant, bacteria) lipids. For instance, the complex lipid particle comprises about 25-95% w/w, about 30-95% w/w, about 35-95% w/w, about 40-95% w/w, about 45-95% w/w, about 50-95% w/w, about 55-95% w/w, about 60-95% w/w, about 65-95% w/w, about 70-95% w/w, about 75-95% w/w, about 80-95% w/w, or about 85-95% w/w of the natural (e.g., plant, bacteria) lipids based on the amounts of total lipids in the complex lipid formulation.

The complex lipid particle may contain 3-1000 lipids extracted from one or more natural (e.g., plant, bacteria) sources. In some embodiments, the natural source is a plant, plant extract, or fragment or part of a plant. In some embodiments, the natural source is a bacteria, bacteria fragment or part of a bacteria. In some embodiments, the natural source is lemon. In some embodiments the natural source is soy. In other embodiments, the natural source is E. coli.

In some embodiments, the complex lipid particle contains at least 10 natural lipids belonging to one or more of the classes selected from the group consisting of fatty acyls (FA), fatty acyl conjugates, phospholipids, glycerolipids, glycolipids, glycerophospholipids, sphingolipids, waxes, and sterol. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 natural lipids belonging to one or more of the classes selected from the group consisting of fatty acyls (FA), fatty acyl conjugates, phospholipids, glycerolipids, glycolipids, glycerophospholipids, sphingolipids, waxes, and sterol. In some embodiments, the complex lipid particle contains lipids from at least two or at least three of these different classes.

In some embodiments, the complex lipid particle contains at least 10 natural lipids belonging to one or more of the classes selected from the group consisting of glycerolipid, sphingolipid, and sterol. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 natural lipids belonging to one or more of the classes selected from the group consisting of glycerolipid, sphingolipid, and sterol. In some embodiments, the complex lipid particle contains lipids from at least two or at least three of these different classes.

In some embodiments, the complex lipid particle may contain one or more glycerolipids (GL) or glycerophospholipids (GP), which may also include glycolipids.

In some embodiments, the complex lipid particle may contain one or more glycerolipids selected from the group consisting of phospholipids (PL), galactolipids, triacylglycerols (TG), and sulfolipids (SL). In some embodiments, the CLPs contains one or more glycerophospholipids (GP) selected from the group consisting of phosphatidylcholines (PC), phosphatidylethanolamines (PE), phosphatidylserines (PS), and phosphatidylinositols (PI). In some embodiments, the complex lipid particle contains one or more sphingolipids (SP) selected from the group consisting of sulfolipids (SL), glycosyl inositol phosphoryl ceramides (GIPC), glucosylceramides (GCer), ceramides (Cer), and free long-chain bases (LCB). In some embodiments, the complex lipid particle contains one or more phytosterols selected from the group consisting of campesterol, stigmasterol, β-sitosterol, Δ5-avenasterol, brassicasterol, avenasterol, 4-desmethyl sterol, 4α-monomethyl sterol, Δ5-sterol, Δ7-sterol, α-spinasterol, Δ5,Δ7-sterol, phytostanol, and sitosterol.

The CLP may contain one or more natural lipids belonging to one or more classes or sub-classes selected from the group consisting of fatty acids, fatty esters, fatty aldehydes, fatty amides, acyclic oxylipins, cyclic oxylipins, glycerolipids, monoradylglycerols, diradylglycerols, triradylglycerols, estolides, glycosylmonoacylglycerols, sulfoquinovosylmonoacylglycerols, monogalactosylmonoacylglycerol, digalactosylmonoacylglycerol, sulfoquinovosyldiacylglycerols, monogalactosyldiacylglycerol, digalactosyldiacylglycerol, glycosyldiacylglycerols, glyceropphospholipids, phospholipids, lysophospholipids, phosphatidylinositol phosphates, n-modified phospholipids, oxygenated/oxidized phospholipids, shingolipids, sphingoid bases, ceramides, phosphocereamides, glycophingolipids, sterols, cholesterol, cholesteryl ester, steryl esters, bile acids, sterylglycosides, and acylsterylglycosides. The complex lipid particle may contain one or more natural lipids belonging to one or more of the classes or sub-classes selected from the group consisting of the classes or sub-classes listed above. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 natural lipids belonging to one or more of the classes or sub-classes selected from the group consisting of the classes or sub-classes listed above.

In some embodiments, the CLP contains one or more natural lipids belonging to one or more of the sub-classes selected from the group consisting of acyl diacylglyceryl glucuronides, acylhexosylceramides, acylsterylglycosides, bile acids, acyl carnitines, cholesteryl esters, ceramides, cardiolipins, coenzyme Qs, diacylglycerols, digalactosyldiacylglycerols, diacylglyceryl glucuronides, dilysocardiolipins, fatty acids, fatty acid esters of hydroxyl fatty acids, hemibismonoacylglycerophosphates, hexosylceramides, lysophosphatidic acids, lysophophatidylcholines, lysophosphatidylethanolamines, N-acyl-lysophosphatidylethanolamines, lysophosphatidylglycerols, lysophosphatidylinositols, lysophosphatidylserines, monogalactosyldiacylglycerols, lysocardiolipins, N-acyl ethanolaminess, N-acyl glycines, N-acyl glycyl serines, phosphatidic acids, phosphatidylcholines, phosphatidylethanolamines, phosphatidylethanols, phosphatidylglycerols, phosphatidylinositols, ceramide phosphoinositols, phosphatidylmethanols, phosphatidylserines, steryl esters, stigmasterols, sulfatides, sulfonolipids, sphingomyelins, sulfoquinovosyl diacylglycerosl, sterols, and triacylglycerols. In some embodiments, the complex lipid particle contains at least 10 natural (e.g., plant, bacteria) lipids belonging to one or more of the sub-classes selected from the group consisting of the sub-classes listed above. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 natural lipids belonging to one or more of the sub-classes selected from the group consisting of the sub-classes listed above.

The complex lipid particle may contain 10 or more natural lipids belonging to one or more of the sub-classes selected from the group consisting of acylsterylglycosides, ceramides, digalactosyldiacylglycerols, diacylglyceryl glucuronides, hemibismonoacylglycerophosphates, hexosylceramides, lysophophatidylcholines, lysophosphatidylethanolamines, monogalactosyldiacylglycerols, phosphatidylcholines, phosphatidylethanolamines, phosphatidylethanols, phosphatidylglycerols, phosphatidylinositols, sulfoquinovosyl diacylglycerosl, and sterols. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 400, at least 500, at least 600, at least 700, or at least 800 natural lipids belonging to one or more of the sub-classes selected from the group consisting of the sub-classes listed above.

The complex lipid particle may contain natural lipids from at least 5, at least 6, at least 7, at least 8, at least 9, or at least 10 different classes or sub-classes of lipids from the natural sources. In some embodiments, the complex lipid particle contains natural lipids from at least 5, at least 6, at least 7, at least 8, at least 9, or at least 10 different classes or sub-classes of lipids from a single natural source (e.g., from only the plant source, or from only the bacteria source). In some embodiments, the CLP may contain natural lipids from only one class or only one sub-class of lipids from the natural sources.

The identity (and class and subclass) and the amounts of the lipids extracted from the natural source(s) can be analyzed by lipidomic analysis by solubilizing the lipid extracts or complex lipid particles in compatible solvents and analyzing by a mass spectrometry (e.g., MS/MS). Other known methods, such as charged aerosol detection (CAD) (e.g., HPLC-CAD, normal-phase high-performance liquid chromatography (NP-HPLC-CAD), or reversed-phase high-performance liquid chromatography (RP-HPLC-CAD)), may also be used.

The complex lipid particle may comprise all or a fraction of the lipid species present in the lipid structure from the particular natural source(s), e.g., it may contain at least 10%, at least 20%, at least 30%, at least 40%, at least 50%, at least 60%, at least 70%, at least 80%, at least 90%, or virtually 100% of the lipid species present in the lipid structure from the particular natural source(s).

The complex lipid particle may comprise reduced or minimized protein matter endogenous to the one or more natural sources. For instance, the complex lipid particle may contain less than 50% w/w, less than 45% w/w, less than 40% w/w, less than 35% w/w, less than 30% w/w, less than 25% w/w, less than 20% w/w, less than 15% w/w, less than 10% w/w, less than 9% w/w, less than 8% w/w, less than 7% w/w, less than 6% w/w, less than 5% w/w, less than 4% w/w, less than 3% w/w, less than 2% w/w, less than 1% w/w, less than 0.5% w/w, less than 0.1% w/w, or essentially free of protein matter endogenous to the one or more natural sources. In some instances, the lipid bilayer of the complex lipid particle does not contain proteins. To calculate % w/w of residual protein matter endogenous to the one or more natural sources, protein concentration is divided by the concentration of the natural lipid extract and then multiplied by 100. Alternatively, % w/w is calculated as the percent of the mass of total protein endogenous to the one or more natural sources based on the mass of the total lipid extract.

The complex lipid particle may comprise reduced or minimized residual dsDNA matter endogenous to the one or more natural sources. For instance, the complex lipid particle may contain less than 15% w/w, less than 10% w/w, less than 5% w/w, less than 1% w/w, less than 0.5% w/w, less than 0.1% w/w, less than 0.05% w/w, less than 0.01% w/w, less than 0.005% w/w, less than 0.001% w/w, or essentially free of residual dsDNA matter endogenous to the one or more natural sources. In some instances, the lipid bilayer of the complex lipid particle does not contain residual dsDNA. To calculate % w/w of residual dsDNA matter endogenous to the one or more natural sources, total adjusted dsDNA concentration is divided by the concentration of the natural lipid extract and then multiplied by 100. Alternatively, % w/w is calculated as the percent of the mass of total residual dsDNA endogenous to the one or more natural sources based on the mass of the total lipid extract.

In some embodiments, the complex lipid particle further incorporates a synthetic structural lipid such as a neutral lipid. In some embodiments, the structural lipid component of the complex lipid particle may comprise between 10% w/w and 99% w/w structural lipids derived from a synthetic lipid structure, e.g., it may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a synthetic lipid structure.

In addition to the exogenous ionizable lipid, the complex lipid particle may further comprise at least two other exogenous lipids. The complex lipid particle may include at least 1% w/w, at least 2% w/w, at least 5% w/w, at least 10% w/w, at least 15% w/w, at least 20% w/w, at least 25% w/w, at least 30% w/w, at least 40% w/w, at least 50% w/w, at least 60% w/w, at least 70% w/w, at least 80% w/w, at least 90% w/w, or about 95% w/w exogenous lipids. Exemplary exogenous lipids include ionizable lipids, synthetic structural lipids, sterols, and PEG-lipid conjugate. The complex lipid particle may further comprise at least two exogenous lipids. In some embodiments, the complex lipid particle contains an ionizable lipid, a sterol, and PEG-lipid conjugate. Additional exogenous lipids suitable for being included in the complex lipid particle are described herein below.

In some embodiments, the CLPs contain natural lipids comprising fatty acid-derived tails, said fatty acid-derived tails of the natural lipids being:
about 5 to 20% of fatty acid 16:0
about 0 to 10% of fatty acid 18:1 (C₉)
about 0 to 10% of fatty acid 18:1 (C₇)
about 5 to 30% of fatty acid 18:2
about 2 to 20% of fatty acid 18:3.

In some embodiments, the CLPs contain phosphatidylcholine (PC) lipids comprising fatty acid-derived tails, said fatty acid-derived tails of the PC lipids being:
about 10 to 20% of fatty acid 16:0
about 2 to 5% of fatty acid 18:0
about 7 to 15% of fatty acid 18:1
about 50 to 75% of fatty acid 18:2
about 2 to 10% of fatty acid 18:3.

In some embodiments, the CLPs contain phosphatidylethanolamines (PE) lipids comprising fatty acid-derived tails, said fatty acid-derived tails of the PE lipids being:
about 0.25 to 5% of fatty acid 14:0
about 25 to 45% of fatty acid 16:0
about 5 to 15% of fatty acid 16:1
about 10 to 25% of fatty acid 17:0
about 25 to 45% of fatty acid 18:1
about 2 to 7% of fatty acid 19:0.

In some embodiments, the CLPs contain natural lipids belonging to the sub-classes of phosphatidylethanolamines, phosphatidylglycerol, and cardiolipin, and comprising:
about 50 to 75 wt/wt % of phosphatidylethanolamines (PE)
about 15 to 30 wt/wt % of phosphatidylglycerol (PG)
about 5 to 15 wt/wt % of cardiolipin (CL).

In some embodiments, the CLPs contain natural lipids comprising
about 10 to 50 wt/wt % of phosphatidylcholines (PC)
about 5 to 50 wt/wt % of phosphatidylethanolamines (PE)
about 0 to 15 wt/wt % of triacylglycerol (TG)
about 5 to 35 wt/wt % of hexosylceramides (HexCer)
about 0 to 5 wt/wt % of phosphatidylglycerol (PG)
about 0 to 7 wt/wt % of phosphatidylserines (PS)
about 0 to 10 wt/wt % of phosphatidylinositols (PI)
about 0 to 5 wt/wt % of cardiolipin (CL).

In some embodiments, the complex lipid particle contains less than 12% w/w of chloroplast endogenous to the one or more natural sources. In some embodiments, the complex lipid particle contains less than 20% w/w, less than 15% w/w, less than 10% w/w, less than 5% w/w, less than 1% w/w, less than 0.5% w/w, or less than 0.1% w/w of chloroplast endogenous to the one or more natural sources.

In some embodiments, the complex lipid particle contains less than 5% w/w of exogenous antioxidant.

In some embodiments, the CLPs contain natural lipids comprising about 0 to 20 wt/wt % of cardiolipin (CL).

### Natural Messenger Packs (NMPs)

A plurality of NMPs in a modified NMP formulation may be loaded with the exogenous peptide, polypeptide, or protein such that at least 5%, at least 10%, at least 15%, at least 25%, at least 50%, at least 75%, at least 90%, or at least 95% of NMPs in the plurality of NMPs encapsulate the exogenous peptide, polypeptide, or protein. In some embodiments the NMP is derived from an arthropod, fungi, archaea, plant, or bacteria. For instance, one example of NMP derived from a plant source is a plant NMP, which may be referred to as a PMP, which is a lipid (e.g., lipid bilayer, unilamellar, or multilamellar structure) structure that includes a plant EV, or segment, portion, or extract (e.g., lipid extract) thereof. Additional descriptions about PMPs can be found in the section “Plant Messenger Pack (PMPs),” in PCT Application No. PCT/US22/47107, filed on Oct. 19, 2022, which is incorporated herein by reference in its entirety.

NMPs can include Arthropod, Plant, Fungi, Archaea, or Bacteria EVs, or segments, portions, or extracts, thereof, in which the EVs are about 5-2000 nm in diameter. For example, the NMP can include an EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the NMP includes a Arthropod, Plant, Fungi, Archaea, or Bacteria EV, or segment, portion, or extract thereof, that has a mean diameter of about 5-1400 nm, 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the Arthropod, Plant, Fungi, Archaea, or Bacteria EV, or segment, portion, or extract thereof, has a mean diameter of about 50-200 nm. In certain instances, the EV, or segment, portion, or extract thereof, has a mean diameter of about 50-300 nm. In certain instances, the EV, or segment, portion, or extract thereof, has a mean diameter of about 200-500 nm. In certain instances, the EV, or segment, portion, or extract thereof, has a mean diameter of about 30-150 nm.

In some instances, the NMP may include a Arthropod, Plant, Fungi, Archaea, or Bacteria EV, or segment, portion, or extract thereof, that has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, or at least 1300. In some instances, the NMP includes a Arthropod, Fungi, Archaea, or Bacteria EV, or segment, portion, or extract thereof, that has a mean diameter less than 1400 nm, less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the EVs, or segment, portion, or extract thereof.

In some instances, the NMP may include an Arthropod, Plant, Fungi, Archaea, or Bacteria EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the NMP may include a Arthropod, Fungi, Archaea, or Bacteria EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³). In some instances, the NMP may include a Arthropod, Plant, Fungi, Archaea, or Bacteria EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the NMP may include a Arthropod, Fungi, Archaea, or Bacteria EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

In some instances, the NMP can have the same size as the Arthropod, Plant, Fungi, Archaea, or Bacteria EV or segment, extract, or portion thereof. Alternatively, the NMP may have a different size than the initial EV from which the NMP is produced. For example, the NMP may have a diameter of about 5-2000 nm in diameter. For example, the NMP can have a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1200 nm, about 1200-1400 nm, about 1400-1600 nm, about 1600-1800 nm, or about 1800-2000 nm. In some instances, the NMP may have a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 1200 nm, at least 1400 nm, at least 1600 nm, at least 1800 nm, or about 2000 nm. A variety of methods (e.g., a dynamic light scattering method) standard in the art can be used to measure the particle diameter of the NMPs. In some instances, the size of the NMP is determined following loading of heterologous functional agents, or following other modifications to the NMPs.

In some instances, the NMP may have a mean surface area of 77 nm² to 1.3×10⁷ nm² (e.g., 77-100 nm², 100-1000 nm², 1000-1×10⁴ nm², 1×10⁴-1×10⁵ nm², 1×10⁵-1×10⁶ nm², or 1×10⁶-1.3×10⁷ nm²). In some instances, the NMP may have a mean volume of 65 nm³ to 4.2×10⁹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-1×10⁹ nm³, or 1×10⁹-4.2×10⁹ nm³). In some instances, the NMP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 1×10⁷ nm²). In some instances, the NMP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 1×10⁹ nm³, at least 2×10⁹ nm³, at least 3×10⁹ nm³, or at least 4×10⁹ nm³).

In some instances, the NMP may include an intact Arthropod, Plant, Fungi, Archaea, or Bacteria EV. In some embodiments, the NMP may include a non-plant natural source such as algae or animal-derived organs EV, or segment, portion, or extract thereof. Alternatively, the NMP may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the plurality of NMPs can include a plurality of intact EVs, a plurality of EV segments, portions, or extracts, or a mixture of intact and segments of EVs. One skilled in the art will appreciate that the ratio of intact to segmented EVs will depend on the particular isolation method used. For example, grinding or blending an Arthropod, Fungi, Plant, Archaea, or Bacteria, or part thereof, may produce NMPs that contain a higher percentage of EV segments, portions, or extracts than a non-destructive extraction method, such as vacuum-infiltration.

In instances where, the NMP includes a segment, portion, or extract of a Arthropod, Fungi, Archaea, or Bacteria EV, the EV segment, portion, or extract may have a mean surface area less than that of an intact vesicle, e.g., a mean surface area less than 77 nm², 100 nm², 1000 nm², 1×10⁴ nm², 1×10⁵ nm², 1×10⁶ nm², or 3.2×10⁶ nm²). In some instances, the EV segment, portion, or extract has a surface area of less than 70 nm², 60 nm², 50 nm², 40 nm², 30 nm², 20 nm², or 10 nm²). In some instances, the NMP may include a Arthropod, Fungi, Archaea, or Bacteria EV, or segment, portion, or extract thereof, that has a mean volume less than that of an intact vesicle, e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

In instances where the NMP includes an extract of a Arthropod, Plant, Fungi, Archaea, or Bacteria EV, e.g., in instances where the NMP includes lipids extracted (e.g., with chloroform or ethanol) from a EV, the NMP may include at least 1%, 2%, 5%, 10%, 20%, 30%, 40%, 50%, 60%, 70%, 80%, 90%, 95%, 99%, or more than 99% of lipids extracted (e.g., with chloroform or with ethanol) from a Arthropod, Fungi, Archaea, or Bacteria EV. The NMPs in the plurality may include Arthropod, Plant, Fungi, Archaea, or Bacteria EV segments and/or EV-extracted lipids or a mixture thereof.

### Production of NMPs

NMPs may be produced from Arthropod, Fungi, Plant, Archaea, or Bacteria EVs, or a segment, portion or extract (e.g., lipid extract) thereof, that occur naturally in Arthropod, Fungi, Plant, Archaea, or Bacteria, or parts thereof, including tissues or cells. In some embodiments, the NMP may include a non-plant natural source such as algae or animal-derived organs EV, or segment, portion, or extract thereof. An exemplary method for producing NMPs includes (a) providing an initial sample from a source, or a part thereof, wherein the source or part thereof comprises EVs; and (b) isolating a crude NMP fraction from the initial sample, wherein the crude NMP fraction has a decreased level of at least one contaminant or undesired component from the source or part thereof relative to the level in the initial sample. The method can further include an additional step (c) comprising purifying the crude NMP fraction, thereby producing a plurality of pure NMPs, wherein the plurality of pure NMPs have a decreased level of at least one contaminant or undesired component from the Arthropod, Fungi, Archaea, or Bacteria or part thereof relative to the level in the crude EV fraction. Each production step is discussed in further detail, below.

For instance, PMPs may be produced from plant EVs, or a segment, portion or extract (e.g., lipid extract) thereof, that occur naturally in plants, or parts thereof, including plant tissues or plant cells. An exemplary method for producing PMPs includes (a) providing an initial sample from a plant, or a part thereof, wherein the plant or part thereof comprises EVs; and (b) isolating a crude PMP fraction from the initial sample, wherein the crude PMP fraction has a decreased level of at least one contaminant or undesired component from the plant or part thereof relative to the level in the initial sample. The method can further include an additional step (c) comprising purifying the crude PMP fraction, thereby producing a plurality of pure PMPs, wherein the plurality of pure PMPs have a decreased level of at least one contaminant or undesired component from the plant or part thereof relative to the level in the crude EV fraction. Each production step is discussed in further detail, below. Exemplary methods regarding the isolation and purification of NMPs (e.g., plant NMPs, PMPs) is found, for example, in Rutter and Innes, Plant Physiol. 173(1): 728-741, 2017; Rutter et al, Bio. Protoc. 7(17): e2533, 2017; Regente et al, J of Exp. Biol. 68(20): 5485-5496, 2017; Mu et al, Mol. Nutr. Food Res., 58, 1561-1573, 2014, and Regente et al, FEBS Letters. 583: 3363-3366, 2009, each of which is herein incorporated by reference. Additional descriptions about the production of PMPs can be found in the section “Production of PMPs,” in PCT Application No. PCT/US22/47107, filed on Oct. 19, 2022, which is incorporated herein by reference in its entirety.

For example, a plurality of NMPs may be isolated from a Arthropod, Fungi, Plant, Archaea, or Bacteria by a process which includes the steps of: (a) providing an initial sample from a source, or a part thereof, wherein the source or part thereof comprises EVs; (b) isolating a crude NMP fraction from the initial sample, wherein the crude NMP fraction has a decreased level of at least one contaminant or undesired component from the Arthropod, Fungi, Plant, Archaea, or Bacteria or part thereof relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude NMP fraction, thereby producing a plurality of pure NMPs, wherein the plurality of pure NMPs have a decreased level of at least one contaminant or undesired component from the source or part thereof relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The NMPs provided herein can include an Arthropod, Fungi, Archaea, or Bacteria EV, or segment, portion, or extract thereof, isolated from a variety of sources.

For instance, the plant NMPs, PMPs, can include a plant EV, or segment, portion, or extract thereof, produced from a variety of plants. PMPs may be produced from any genera of plants (vascular or nonvascular), including but not limited to angiosperms (monocotyledonous and dicotyledonous plants), gymnosperms, ferns, selaginellas, horsetails, psilophytes, lycophytes, algae (e.g., unicellular or multicellular, e.g., archaeplastida), or bryophytes. In certain instances, PMPs can be produced using a vascular plant, for example monocotyledons or dicotyledons or gymnosperms. For example, PMPs can be produced using alfalfa, apple, Arabidopsis, banana, barley, a Brassica species (e.g., Arabidopsis thaliana or Brassica napus), canola, castor bean, chicory, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat or vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, or wheat.

PMPs (i.e., plant NMPs) may be produced using a whole plant (e.g., a whole rosettes or seedlings) or alternatively from one or more plant parts (e.g., leaf, seed, root, fruit, vegetable, pollen, phloem sap, or xylem sap). For example, PMPs can be produced using shoot vegetative organs/structures (e.g., leaves, stems, or tubers), roots, flowers and floral organs/structures (e.g., pollen, bracts, sepals, petals, stamens, carpels, anthers, or ovules), seed (including embryo, endosperm, or seed coat), fruit (the mature ovary), sap (e.g., phloem or xylem sap), plant tissue (e.g., vascular tissue, ground tissue, tumor tissue, or the like), and cells (e.g., single cells, protoplasts, embryos, callus tissue, guard cells, egg cells, or the like), or progeny of same. For instance, the isolation step may involve (a) providing a plant, or a part thereof. In some examples, the plant part is an Arabidopsis leaf. The plant may be at any stage of development. For example, the PMPs can be produced using seedlings, e.g., 1-week, 2-week, 3-week, 4-week, 5-week, 6-week, 7-week, or 8-week old seedlings (e.g., Arabidopsis seedlings). Other exemplary PMPs can include PMPs produced using roots (e.g., ginger roots), fruit juice (e.g., grapefruit juice), vegetables (e.g., broccoli), pollen (e.g., olive pollen), phloem sap (e.g., Arabidopsis phloem sap), or xylem sap (e.g., tomato plant xylem sap). In some embodiments, the PMPs are produced from algae or lemon.

NMPs may be isolated from any genera of Arthropod, Fungi, Archaea, or Bacteria, including but not limited to crabs, crawfish, shrimp, spiders, scorpions, crickets, grasshoppers, beetles, millipedes, ticks, mites, centipedes, ants, wasps, dragonflies, flies, gnats, other insects and crustaceans, yeast, mushrooms, puffballs, stinkhorns, boletes, smuts, bunts, bracket fungi, jelly fungi, toadstools, molds, rusts, earth stars, chanterelles, ergot, pyrolobus, picrophilus, methanogens, crenarchaeota, nanoarchaeota, ignicoccus, cenarchaeum, halophiles, Escherichia, Acinetobacter, Agrobacterium, Anabaena, Anaplasma, Aquifex, Azoarcus, Azospirillum, Azotobacter, Bartonella, Bordetella, Bradyrhizobium, Brucella, Buchnera, Burkholderia, Candidatus, Chromobacterium, Coxiella, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylobacterium, Methylococcus, Neisseria, Nitrosomonas, Nostoc, Photobacterium, Photorhabdus, Phyllobacterium, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Bifidobacterium, Clostridium, Corynebacterium, Deinococcus, Enterococcus, Exiguobacterium, Geobacillus, Lactobacillus, Listeria, Leuconostoc, Moorella, Oceanobacillus, Rhizobium, Rickettsia, Staphylococcus, Streptococcus, Symbiobacterium, or Thermoanaerobacter.

NMPs may be produced from a whole Arthropod, Fungi, Archaea, or Bacteria (e.g., a whole insect, spider, crustacean, fungi, or single cell of archaea or bacteria) or alternatively from one or more source parts (e.g., segments, organs, eggs, spores, mycelium, tissue, membrane or cell wall). For example, NMPs can be produced from organs/structures/tissues/cell cultures (e.g., body segments, appendages, organs, eggs, exoskeleton, embryos, spores, mycelium, hyphae, thallus, suspension cultures, cell walls, inner or outer membranes, gametophytes, sporophytes, polymerases, glycerol-ether lipids, metabolic products, flagella, pili, ribosomes or organelles) or progeny of same. The source may be at any stage of development. In some embodiments, the NMP is produced from an insect or fungi, (e.g. cricket, yeast, or mushroom). In some embodiments, the NMP is produced from a bacteria or archaea (e.g. E. coli). In some embodiments, the NMP is produced from algae (e.g. kelp or chlorella). In some embodiments, the NMP is produced from an animal organ (e.g. brain or blood).

NMPs can be produced from a plant, Arthropod, Fungi, Archaea, or Bacteria, or part thereof, by a variety of methods. Any method that allows release of the EV-containing fraction of a source, or an otherwise extracellular fraction that contains NMPs comprising secreted EVs (e.g., cell culture media) is suitable in the present methods. EVs can be separated from the source or source part by either destructive (e.g., grinding or blending) or non-destructive (washing or vacuum infiltration) methods. For instance, the plant, Arthropod, Fungi, Archaea, or Bacteria, or part thereof, can be vacuum-infiltrated, ground, blended, or a combination thereof to isolate EVs from the source or source part, thereby producing NMPs. For instance, the isolating step may involve (b) isolating a crude NMP fraction from the initial sample (e.g., a plant, Arthropod, Fungi, Archaea, or Bacteria or part, or a sample derived from a plant, Arthropod, Fungi, Archaea, or Bacteria or part), wherein the crude NMP fraction has a decreased level of at least one contaminant or undesired component from the source or part thereof relative to the level in the initial sample; wherein the isolating step involves vacuum infiltrating the plant, Arthropod, Fungi, Archaea, or Bacteria (e.g., with a vesicle isolation buffer) to release and collect the desired fraction. Alternatively, the isolating step may involve (b) grinding or blending the source to release the EVs, thereby producing NMPs.

Upon isolating the plant, Arthropod, Fungi, Archaea, or Bacteria EVs, thereby producing NMPs, the NMPs can be separated or collected into a crude NMP fraction. For instance, the separating step may involve separating the plurality of NMPs into a crude NMP fraction using centrifugation (e.g., differential centrifugation or ultracentrifugation) and/or filtration to separate the NMP-containing fraction from large contaminants, including tissue debris, cells, or cell organelles. As such, the crude NMP fraction will have a decreased number of large contaminants, including, for example, tissue debris, cells, or cell organelles (e.g., nuclei, mitochondria, etc.), as compared to the initial sample from the source or source part.

The crude NMP fraction can be further purified by additional purification methods to produce a plurality of pure NMPs. For example, the crude NMP fraction can be separated from other source components by ultracentrifugation, e.g., using a density gradient (iodixanol or sucrose), size-exclusion, and/or use of other approaches to remove aggregated components (e.g., precipitation or size-exclusion chromatography). The resulting pure NMPs may have a decreased level of contaminants or undesired components from the source (e.g., one or more non-NMP components, such as protein aggregates, nucleic acid aggregates, protein-nucleic acid aggregates, free lipoproteins, lipido-proteic structures), nuclei, cell wall components, cell organelles, or a combination thereof) relative to one or more fractions generated during the earlier separation steps, or relative to a pre-established threshold level, e.g., a commercial release specification. For example, the pure NMPs may have a decreased level (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) of source organelles or cell wall components relative to the level in the initial sample. In some instances, the pure NMPs are substantially free (e.g., have undetectable levels) of one or more non-NMP components, such as protein aggregates, nucleic acid aggregates, protein-nucleic acid aggregates, free lipoproteins, lipido-proteic structures), nuclei, cell wall components, cell organelles, or a combination thereof. Further examples of the releasing and separation steps can be found in WO 2021/041301. The NMPs may be at a concentration of, e.g., 1×10⁹, 5×10⁹, 1×10¹⁰, 5×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, or more than 1×10¹³ NMPs/mL.

For example, protein aggregates may be removed from isolated NMPs. For example, the isolated NMP solution can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the isolated NMP solution can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example, NaCl can be added to the isolated NMP solution until it is at, e.g., 1 mol/L. The solution can then be filtered to isolate the NMPs. Alternatively, aggregates are solubilized by increasing the temperature. For example, the isolated NMPs can be heated under mixing until the solution has reached a uniform temperature of, e.g., 50° C. for 5 minutes. The NMP mixture can then be filtered to isolate the NMPs. Alternatively, soluble contaminants from NMP solutions can be separated by size-exclusion chromatography column according to standard procedures, where NMPs elute in the first fractions, whereas proteins and ribonucleoproteins and some lipoproteins are eluted later. The efficiency of protein aggregate removal can be determined by measuring and comparing the protein concentration before and after removal of protein aggregates via BCA/Bradford protein quantification. In some embodiments, protein aggregates are removed before the exogenous peptide, polypeptide, or protein is encapsulated by the NMP. In other embodiments, protein aggregates are removed after the exogenous peptide, polypeptide, or protein is encapsulated by the NMP.

In some embodiments, the preparation of NMPs from natural sources is through an ethanol extraction method. In some aspects, a 3:2 ethyl acetate:ethanol solvent aids in extraction. In some embodiments, the preparation of NMPs from natural sources is through a modified Matyash extraction method. In some aspects, a 1:2 MeOH:MTBE solvent aids in extraction.

Any of the production methods described herein can be supplemented with any quantitative or qualitative methods known in the art to characterize or identify the NMPs at any step of the production process. NMPs may be characterized by a variety of analysis methods to estimate NMP yield, NMP concentration, NMP purity, NMP composition, or NMP sizes. NMPs can be evaluated by a number of methods known in the art that enable visualization, quantitation, or qualitative characterization (e.g., identification of the composition) of the NMPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify EV markers present on the NMP. To aid in analysis and characterization, of the NMP fraction, the NMPs can additionally be labelled or stained. For example, the NMPs can be stained with 3,3′-dihexyloxacarbocyanine iodide (DIOC₆), a fluorescent lipophilic dye, PKH67 (Sigma Aldrich); Alexa Fluor®488 (Thermo Fisher Scientific), or DyLight™ 800 (Thermo Fisher). In the absence of sophisticated forms of nanoparticle tracking, this relatively simple approach quantifies the total membrane content and can be used to indirectly measure the concentration of NMPs (Rutter and Innes, Plant Physiol. 173(1): 728-741, 2017; Rutter et al, Bio. Protoc. 7(17): e2533, 2017). For more precise measurements, and to assess the size distributions of NMPs, nanoparticle tracking, nano flow cytometry, or Tunable Resistive Pulse Sensing can be used.

During the production process, the NMPs can optionally be prepared such that the NMPs are at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the EV level in a control or initial sample. The isolated NMPs may make up about 0.1% to about 100% of the NMP composition, such as any one of about 0.01% to about 100%, about 1% to about 99.9%, about 0.1% to about 10%, about 1% to about 25%, about 10% to about 50%, about 50% to about 99%. In some instances, the composition described herein includes at least any of 0.1%, 0.5%, 1%, 5%, 10%, 20%, 30%, 40%, 50%, 60%, 70%, 80%, 90%, 95%, or more NMPs, e.g., as measured by wt/vol, percent NMP protein composition, and/or percent lipid composition (e.g., by measuring fluorescently labelled lipids)). In some instances, the concentrated agents are used as commercial products, e.g., the final user may use diluted agents, which have a substantially lower concentration of active ingredient. In some embodiments, the composition described herein is formulated as a NMP concentrate formulation, e.g., an ultra-low-volume concentrate formulation. In some embodiments, the NMPs in the composition are at a concentration effective to increase the fitness of an organism, e.g., a plant, an animal, an insect, a bacterium, or a fungus. In other aspects, the NMPs in the composition are at a concentration effective to decrease the fitness of an organism, e.g., a plant, an animal, an insect, a bacterium, or a fungus.

NMPs can be produced from a variety of Arthropod, Fungi, Plant, Archaea, or Bacteria, or one or more parts thereof (e.g., segments, organs, eggs, spores, mycelium, tissue, membrane or cell wall). For example, NMPs can be produced from organs/structures/tissues/cell cultures (e.g., body segments, appendages, organs, eggs, exoskeleton, embryos, spores, mycelium, hyphae, thallus, suspension cultures, cell walls, inner or outer membranes, gametophytes, sporophytes, polymerases, glycerol-ether lipids, metabolic products, flagella, pili, ribosomes or organelles) or progeny of same. The source may be at any stage of development. In some embodiments, the NMP is produced from an insect or fungi, (e.g. cricket, yeast, or mushroom). In some embodiments, the NMP is produced from a bacteria or archaea (e.g. E. coli). In some embodiments, the NMP is produced from an algae (e.g. kelp or chlorella). In some embodiments, the NMP is produced from an animal organ (e.g. brain or blood).

NMPs can be produced and purified by a variety of methods, for example, by using a density gradient (iodixanol or sucrose) in conjunction with ultracentrifugation and/or methods to remove aggregated contaminants, e.g., precipitation or size-exclusion chromatography.

In some instances, the NMPs of the present compositions and methods can be isolated from an Arthropod, Fungi, Archaea, or Bacteria, or part thereof, and used without further modification to the NMP. In other instances, the NMP can be modified prior to use, as outlined further herein. In some instances, the NMPs are PMPs. In some instances, the NMPs of the present compositions and methods can be isolated from a plant, or part thereof, and used without further modification to the NMP. In other instances, the NMP can be modified prior to use, as outlined further herein.

### Lipid Reconstructed Natural Messenger Packs (LNMPs)

A lipid reconstructed NMP (LNMP) is used herein. LNMP refers to a NMP that has been derived from a lipid structure (e.g., a lipid bilayer, unilamellar, multilamellar structure; e.g., a vesicular lipid structure) derived from (e.g., enriched, isolated or purified from) a natural source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LNMP, as is described herein. For instance, a plant LNMP can be referred to as a LPMP, a lipid reconstructed plant messenger pack, which is derived from a plant source.

The method for a lipid reconstructing NMP (e.g., LPMP) may, if desired, further comprise sonication, freeze/thaw treatment, and/or lipid extrusion, e.g., to reduce the size of the reconstituted LNMPs. Alternatively, LNMPs (e.g., LPMPs) may be produced using a microfluidic device (such as a NanoAssemblr® IGNITE™ microfluidic instrument (Precision NanoSystems)).

In some embodiments, the LNMPs (e.g., LPMPs) are produced by a process which comprises the steps of (a) providing a plurality of purified NMPs (e.g., purified PMPs); (b) processing the plurality of NMPs (e.g., PMPs) to produce a lipid film; (c) reconstituting the lipid film in an organic solvent or solvent combination, thereby producing a lipid solution; and (d) processing the lipid solution of step (c) in a microfluidics device comprising an aqueous phase, thereby producing the LNMPs (e.g., LPMPs).

In some instances, processing the plurality of NMPs (e.g., PMPs) to produce a lipid film includes extracting lipids from the plurality of NMPs, e.g., extracting lipids using the Bligh-Dyer method (Bligh and Dyer, J Biolchem Physiol, 37: 911-917, 1959). The extracted lipids may be provided as a stock solution, e.g., a solution in chloroform:methanol. Producing the lipid film may comprise, e.g., evaporation of the solvent with a stream of inert gas (e.g., nitrogen).

### Natural Lipids

A LNMP may comprise between 10% and 100% lipids derived from the lipid structure from the natural source (e.g., lemon or algae), e.g., may contain at least 10%, at least 20%, at least 30%, at least 40%, at least 50%, at least 60%, at least 70%, at least 80%, at least 90%, at least 95%, or 100% lipids derived from the lipid structure from the natural source. A LNMP may comprise all or a fraction of the lipid species present in the lipid structure from the natural source (e.g., lemon or algae), e.g., it may contain at least 10%, at least 20%, at least 30%, at least 40%, at least 50%, at least 60%, at least 70%, at least 80%, at least 90%, or 100% of the lipid species present in the lipid structure from the natural source. A LNMP may comprise none, a fraction, or all of the protein species present in the lipid structure from the natural source (e.g., lemon or algae), e.g., may contain 0%, less than 1%, less than 5%, less than 10%, less than 15%, less than 20%, less than 30%, less than 40%, less than 50%, less than 60%, less than 70%, less than 80%, less than 90%, less than 100%, or 100% of the protein species present in the lipid structure from the natural source (e.g., lemon or algae). In some instances, the lipid bilayer of the LNMP does not contain proteins. In some instances, the lipid structure of the LNMP contains a reduced number of proteins relative to the lipid structure from the natural source.

In some embodiments, the natural lipids of the LNMPs are extracted from a plant source, such as lemon or algae. In some embodiments, the natural lipids of the LNMPs are extracted from a bacteria source, such as Escherichia or Salmonella.

In some embodiments, the natural lipids of the LNMPs are extracted from lemon or algae.

### Exogenous Lipids

The LNMPs may be modified to contain a heterologous agent (e.g., a cell-penetrating agent) that is capable of increasing cell uptake (e.g., animal cell uptake (e.g., mammalian cell uptake, e.g., human cell uptake), plant cell uptake, bacterial cell uptake, or fungal cell uptake) relative to an unmodified LNMP. For example, the modified LNMPs may include (e.g., be loaded with, e.g., encapsulate or be conjugated to) or be formulated with (e.g., be suspended or resuspended in a solution comprising) a cell-penetrating agent, such as an ionizable lipid. Each of the modified LNMPs may comprise at least 1%, 5%, 10%, 20%, 30%, 40%, 50%, 60%, 70%, 80%, 90%, or more than 90% ionizable lipid.

LNMPs may include one or more exogenous lipids, e.g., lipids that are exogenous to the natural source (e.g., originating from a source that is not the source or source part from which the LNMP is produced). The lipid composition of the LNMP may include 0%, less than 1%, or at least 1%, 2%, 5%, 10%, 15%, 20%, 25%, 30%, 40%, 50%, 60%, 70%, 80%, 90%, 95%, or more than 95% exogenous lipid. In some examples, the exogenous lipid (e.g., ionizable lipid) is added to amount to 25% or 40% (w/w) of total lipids in the preparation. In some examples, the exogenous lipid is added to the preparation prior to step (b), e.g., mixed with extracted NMP lipids prior to step (b).

Exemplary exogenous lipids include ionizable lipids. The ionizable lipids in the LNMP compositions herein include one or more from the compounds of groups i)-iv) as described herein.

Exogenous lipids may also include cationic lipids.

In some instances, the exogenous lipid may also include an ionizable lipid or cationic lipid chosen from 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), DLin-MC3-DMA (MC3), dioleoyl-3-trimethylammonium propane (DODAP), DC-cholesterol, DOTAP, Ethyl PC, GL67, DLin-KC2-DMA (KC2), MD1 (cKK-E12), OF2, EPC, ZA3-Ep10, TT3, LP01, 5A2-SC8, Lipid 5 (Moderna), a cationic sulfonamide amino lipid, an amphiphilic zwitterionic amino lipid, DODAC, DOBAQ, YSK05, DOBAT, DOBAQ, DOPAT, DOMPAQ, DOAAQ, DMAP-BLP, DLinDMA, DODMA, DOTMA, DSDMA, DOSPA, DODAC, DOBAQ, DMRIE, DOTAP-cholesterol, GL67A, and 98N12-5 or a combination thereof.

In some embodiments, the exogenous lipid may also include an ionizable lipid or cationic lipid chosen from C12-200, MC3, DODAP, DC-cholesterol, DOTAP, Ethyl PC, GL67, KC2, MD1, OF2, EPC, ZA3-Ep10, TT3, LP01, 5A2-SC8, Lipid 5 (Moderna), a cationic sulfonamide amino lipid, and an amphiphilic zwitterionic amino lipid or a combination thereof. In some embodiments, the ionizable lipid is chosen from C12-200, MC3, DODAP, and DC-cholesterol or combinations thereof. In some instances, the ionizable lipid is an ionizable lipid. In some embodiments, the ionizable lipid is 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200) or (6Z,9Z,28Z,31Z)-Heptatriaconta-6,9,28,31-tetraen-19-yl 4-(dimethylamino)butanoate, DLin-MC3-DMA (MC3). In some instances, the exogenous lipid is a cationic lipid. In some embodiments, the cationic lipid is DC-cholesterol or dioleoyl-3-trimethylammonium propane (DOTAP).

In some instances, the LNMPs comprise at least 1%, 5%, 10%, 20%, 30%, 40%, 50%, 60%, 70%, 80%, 90%, or more than 90% ionizable lipid.

In some instances, the LNMPs comprise a molar ratio of least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 30%-75% ionizable lipid (e.g., about 30%-75% ionizable lipid). In some embodiments, the LNMP comprises 25% C12-200. In some embodiments, the LNMP comprises a molar ratio of 35% C12-200. In some embodiments, the LNMP comprises a molar ratio of 50% C12-200. In some embodiments, the LNMP comprises 40% MC3. In some embodiments, the LNMP comprises a molar ratio of 50% C12-200. In some embodiments, the LNMP comprises 20% or 40% DC-cholesterol. In some embodiments, the LNMP comprises 25% or 40% DOTAP.

The agent may increase uptake of the LNMP as a whole or may increase uptake of a portion or component of the LNMP (e.g., the mRNA or circRNA therapeutic) carried by the LNMP. The degree to which cell uptake is increased may vary depending on the natural source or source part to which the composition is delivered, the LNMP formulation, and other modifications made to the LNMP, For example, the modified LNMPs may have an increased cell uptake (e.g., animal cell uptake, plant cell uptake, bacterial cell uptake, or fungal cell uptake) of at least 1%, 2%, 5%, 10%, 15%, 20%, 30%, 40%, 50%, 60%, 70%, 80%, 90%, or 100% relative to an unmodified LNMP. In some instances, the increased cell uptake is an increased cell uptake of at least 2×-fold, 4×-fold, 5×-fold, 10×-fold, 100×-fold, or 1000×-fold relative to an unmodified LNMP.

In some embodiments, a LNMP that has been modified with an ionizable lipid more efficiently encapsulates a negatively charged a polynucleotide than a LNMP that has not been modified with an ionizable lipid. In some aspects, a LNMP that has been modified with an ionizable lipid has altered biodistribution relative to a LNMP that has not been modified with an ionizable lipid. In some aspects, a LNMP that has been modified with an ionizable lipid has altered (e.g., increased) fusion with an endosomal membrane of a target cell relative to a LNMP that has not been modified with an ionizable lipid.

### Ionizable Lipids

In some embodiments, the ionizable lipid has at least one (e.g., one, two, three, four or all five) of the characteristics listed below:
(i) at least 2 ionizable amines (e.g., at least 2, at least 3, at least 4, at least 5, at least 6, or more than 6 ionizable amines, e.g., 2, 3, 4, 5, 6, 7, 8, 9, 10, 11, 12, or more than 12 ionizable amines);
(ii) at least 3 lipid tails (e.g., at least 3, at least 4, at least 5, at least 6, or more than 6 lipid tails, e.g., 2, 3, 4, 5, 6, 7, 8, 9, 10, 11, 12, or more than 12 lipid tails), wherein each of the lipid tails is independently at least 6 carbon atoms in length (e.g., at least 6, at least 7, at least 8, at least 9, at least 10, at least 11, at least 12, at least 13, at least 14, at least 15, at least 16, at least 17, at least 18, or more than 18 carbon atoms in length, e.g., 6, 7, 8, 9, 10, 11, 12, 13, 14, 15, 16, 17, 18, 19, 20, 21, 22, 23, 24, 25, or more than 25 carbon atoms in length);
(iii) an acid dissociation constant (pKa) of from about 4.5 to about 7.5 (e.g., a pKa of about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 (e.g., a pKa of from about 6.5 and about 7.5 (e.g., a pKa of about 6.5, 6.6, 6.7, 6.8, 6.9, 7.0, 7.1, 7.2, 7.3, 7.4, or 7.5));
(iv) an ionizable amine and a heterorganic group; and
(v) an N:P (amines of ionizable lipid:phosphates of mRNA or circRNA) ratio of at least 3 (or at least 4);

In some embodiments, the ionizable lipid is an ionizable amine and a heterorganic group. In some embodiments, the heterorganic group is hydroxyl. In some embodiments, the heterorganic group comprises a hydrogen bond donor. In some embodiments, the heterorganic group comprises a hydrogen bond acceptor. In some embodiments, the heterorganic group is —OH, —SH, —(CO)H, —CO₂H, —NH₂, —CONH₂, optionally substituted C₁-C₆ alkoxy, or fluorine.

In some embodiments, the ionizable lipid is an ionizable amine and a heterorganic group separated by a chain of at least two atoms.

The ionizable lipid in the LNMP compositions included one of the compounds from group i) to group iv) as discussed below.

### Ionizable Lipid Compounds i)

In some embodiments, the ionizable lipid is represented by the following formula I:

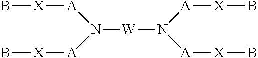

a pharmaceutically acceptable salt thereof, or a stereoisomer of any of the foregoing,
wherein
each A is independently C₁-C₁₆ branched or unbranched alkyl or C₁-C₁₆ branched or unbranched alkenyl, optionally substituted with heteroatom or substituted with OH, SH, or halogen;
each B is independently C₁-C₁₆ branched or unbranched alkyl or C₁-C₁₆ branched or unbranched alkenyl, optionally substituted with heteroatom or substituted with OH, SH, or halogen;
each X is independently a biodegradable moiety; and
W is

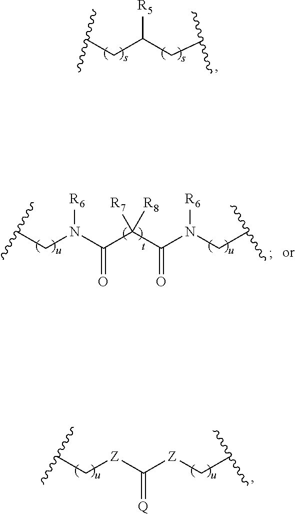

wherein:
   R₅ is OH, SH, or NR₁₀R₁₁;
   each R₆ is independently H, C₁-C₃ branched or unbranched alkyl, C₂-C₃ branched or unbranched alkenyl, or cycloalkyl;
   each R₇ and each R₈ is independently H, C₁-C₃ branched or unbranched alkyl, C₂-C₃ branched or unbranched alkenyl, halogen, OH, SH, or NR₁₀R₁₁, wherein each R₁₀ and R₁₁ is independently H, C₁-C₃ alkyl, or R₁₀ and R₁₁ are taken together to form a heterocyclic ring; R₇ and R₈ are taken together to form a ring;
   each s is independently 1, 2, 3, 4, or 5;
   each u is independently 1, 2, 3, 4, or 5;
   t is 1, 2, 3, 4 or 5;
   each Z is independently absent, O, S, or NR₁₂, wherein R₁₂ is H, C₁-C₇ branched or unbranched alkyl, or C₂-C₇ branched or unbranched alkenyl, and
   Q is O, S, or NR₁₃, wherein each R₁₃ is H, or C₁-C₅ alkyl.

In some embodiments, B is C₃-C₂₀ alkyl.

In some embodiments, W in formula (I) may alternatively be

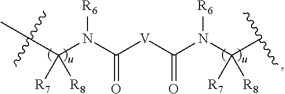

wherein:
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each R₁₀ and R₁₁ is independently H, C₁-C₃ alkyl, or R₁₀ and R₁₁ are taken together to form a heterocyclic ring;
each v is independently 0, 1, 2, 3, 4, or 5;
R₁₇ is OH, SH, or N(CH₃)₂; and
each u is independently 1, 2, 3, 4, or 5.

In some embodiments, W in formula (I) may alternatively be

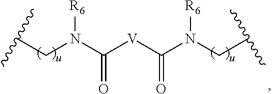

wherein:
V is C₂-C₁₀ alkenylene, C₂-C₁₀ alkynylene, or C₂-C₁₀ heteroalkylene;
each R₆ is independently H, C₁-C₃ branched or unbranched alkyl, C₂-C₃ branched or unbranched alkenyl, or cycloalkyl; and
each u is independently 1, 2, 3, 4, or 5.

In some embodiments, W in formula (I) may alternatively be

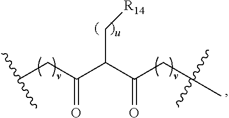

wherein:
R₁₄ is a heterocyclic;
each v is independently 0, 1, 2, 3, 4, or 5; and
each u is independently 1, 2, 3, 4, or 5.

In some embodiments, W in formula (I) may alternatively be

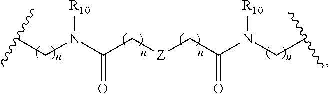

wherein:
Z is O, S, —C((CH₂)ᵥN(R₁₅)₂)—, or N(R₁₅), wherein R₁₅ is H, C₁-C₄ branched or unbranched alkyl, and v is 0, 1, 2, 3, 4, or 5;
each R₁₀ is independently H, or C₁-C₃ alkyl; and
each u is independently 0, 1, 2, 3, 4, or 5.

In some embodiments, W in formula (I) may alternatively be

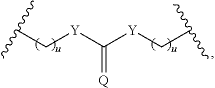

wherein:
each Y is a divalent heterocyclic;
Q is O, S, or NH; and
each u is independently 1, 2, 3, 4, or 5.

In some embodiments, W in formula (I) may alternatively be

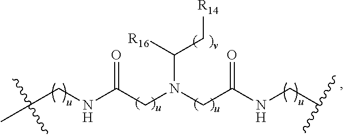

wherein:
R₁₄ is a heterocyclic, NR₁₀R₁₁, C(O)NR₁₀R₁₁, or C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
R₁₆ is H, ═O, ═S, or CN;
each v is independently 0, 1, 2, 3, 4, or 5; and
each u is independently 1, 2, 3, 4, or 5.

In some embodiments, W in formula (I) may alternatively be

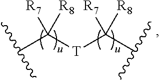

wherein:
T is —NHC(O)O—, —OC(O)NH—, or a divalent heterocyclic optionally substituted with one or more —(CH₂)ᵥOH, —(CH₂)ᵥSH, and/or —(CH₂)ᵥ-halogen groups;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each R₁₀ and R₁₁ is independently H, C₁-C₃ alkyl, or R₁₀ and R₁₁ are taken together to form a heterocyclic ring;
R₁₇ is OH, SH, or N(CH₃)₂;
each v is independently 0, 1, 2, 3, 4, or 5; and
each u is independently 1, 2, 3, 4, or 5.

In some embodiments, W in formula (I) may alternatively be

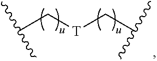

wherein:
T is —NHC(O)O—, —OC(O)NH—, or a divalent heterocyclic; and
each u is independently 1, 2, 3, 4, or 5.

In some embodiments, when Z is not absent, the adjacent R₁ and R₂ cannot be OH, NR₁₀R₁₁, or SH.

In some embodiments, the heterocyclic is a piperazine, piperazine dione, piperazine-2,5-dione, piperidine, pyrrolidine, piperidinol, dioxopiperazine, bis-piperazine, aromatic or heteroaromatic.

In some embodiments, the ionizable lipid is represented by formula (IX):

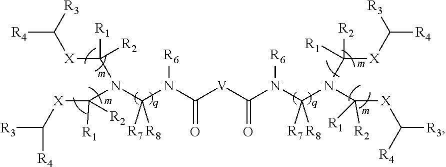

pharmaceutically acceptable salts thereof, and stereoisomers of any of the foregoing, wherein
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each q is independently 2, 3, 4, or 5;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each v is independently 0, 1, 2, 3, 4, or 5, and R₁₇ is OH, SH, or N(CH₃)₂; and
each m is independently 1, 2, 3, 4, 5, 6, 7, 8, 9 or 10.

In some embodiments, V is a branched or unbranched C₂-C₃ alkylene. In some embodiments, V is a C₂-C₃ alkylene substituted with OH. In some embodiments, V is a branched or unbranched C₂-C₃ alkenylene. In some embodiments, each R₆ is independently H or methyl.

In some embodiments, the ionizable lipid is represented by one of the following formulas

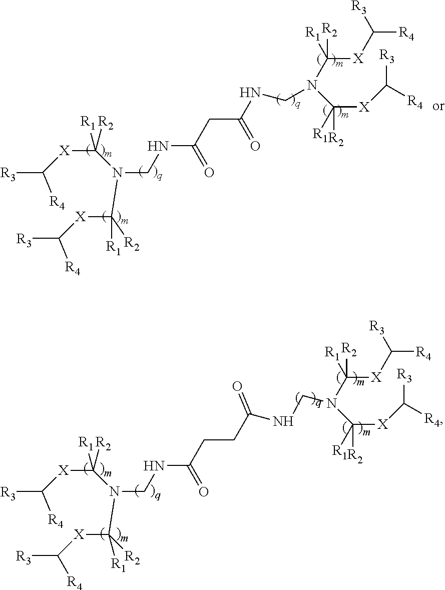

wherein the definition for the variables are the same as those in formula (X).

In some embodiments, the disclosure relates to ionizable lipids of Formula (XI):

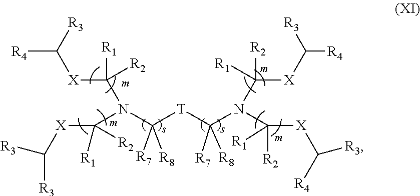

pharmaceutically acceptable salts thereof, and stereoisomers of any of the foregoing, wherein
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each s is independently 1, 2, 3, 4, or 5;
T is —NHC(O)O—, —OC(O)NH—, or a divalent heterocyclic optionally substituted with one or more —(CH₂)ᵥOH, —(CH₂)ᵥSH, —(CH₂)ᵥ-halogen groups,
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein R₁₇ is OH, SH, or N(CH₃)₂;
each v is independently 0, 1, 2, 3, 4, or 5; and
each m is independently 1, 2, 3, 4, 5, 6, 7, 8, 9 or 10.

In some embodiments, T is a divalent heterocyclic (e.g., a divalent piperazine, or a divalent dioxopiperazine) optionally substituted with —(CH₂)ᵥOH, wherein v is independently 0, 1, or 2.

In some embodiments, in each of the above formulas, X is —OC(O)—, —C(O)O—, —SS—, —N(R¹⁸)C(O), —C(O)N(R¹⁸), —C(O—R₁₃)—O—, —C(O)O(CH₂)ₐ—, —OC(O)(CH₂)ₐ—, —C(O)N(R¹⁸)(CH₂)ₐ—, —N(R¹⁸)C(O)(CH₂)ₐ—,

—C(O—R₁₃)—O—(CH₂)ₐ—, wherein each R¹⁸ is independently H, alkyl, alkenyl, cycloalkyl, hydroxyalkyl, or aminoalkyl, each R₁₃ is independently C₃-C₁₀ alkyl, and each a is independently 0-16. In one embodiment, each X is independently —OCO—, —COO—, —NHCO—, or —CONH—. In one embodiment, at least one X is —SS—.

More embodiments of the ionizable lipid of formula (I), in the Ionizable lipid compounds group i), may be found in POT Application No. PCT/US22/50725, filed on Nov. 22, 2022, the content of which is incorporated herein by reference in its entirety. In particular, all the ionizable lipids of formulas (I)-(XII) of PCT Application No. PCT/US22/50725 are suitable for use as the ionizable lipids in this disclosure, and are incorporated herein by reference in its entirety.

Certain exemplary ionizable lipid compounds disclosed herein are set forth in Table I below.

TABLE I
Exemplary ionizable lipid compounds.

| Lipid | |
|---|---|
| No. | Structure |
| 2303 | 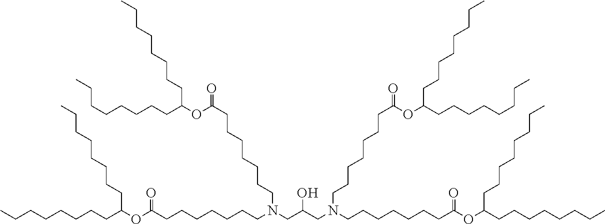 [SMILES: CCCCCCCCC(CCCCCCCC)OC(=O)CCCCCCCN(CCCCCCCC(=O)OC(CCCCCCCC)CCCCCCCC)CC(O)CN(CCCCCCCC(=O)OC(CCCCCCCC)CCCCCCCC)CCCCCCCC(=O)OC(CCCCCCCC)CCCCCCCC] |
| 2302 | 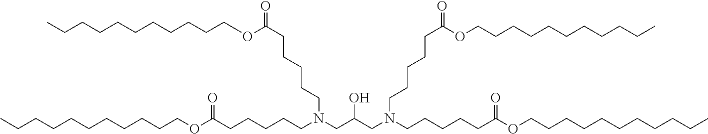 |
| 2299 | 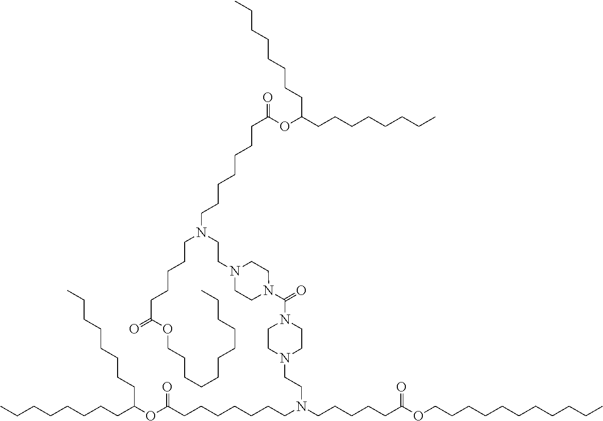 [SMILES: CCCCCCCCCCCOC(=O)CCCCCN(CCCCCCCC(=O)OC(CCCCCCCC)CCCCCCCC)CCN1CCN(C(=O)N2CCN(CCN(CCCCCCCC(=O)OC(CCCCCCCC)CCCCCCCC)CCCCCC(=O)OCCCCCCCCCCC)CC2)CC1] |
| 2289 | 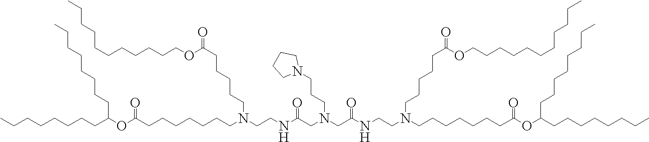 [SMILES: CCCCCCCCCCCOC(=O)CCCCCN(CCCCCCCC(=O)OC(CCCCCCCC)CCCCCCCC)CCNC(=O)CN(CCCN1CCCC1)CC(=O)NCCN(CCCCCCCC(=O)OC(CCCCCCCC)CCCCCCCC)CCCCCC(=O)OCCCCCCCCCCC] |
| 2288 | 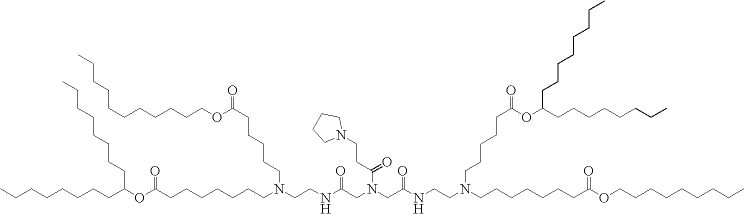 [SMILES: CCCCCCCCCCCOC(=O)CCCCCN(CCCCCCCC(=O)OC(CCCCCCCC)CCCCCCCC)CCNC(=O)CN(CC(=O)NCCN(CCCCCCCC(=O)OCCCCCCCCC)CCCCCC(=O)OC(CCCCCCCC)CCCCCCCC)C(=O)CCN1CCCC1] |
| 2286 | 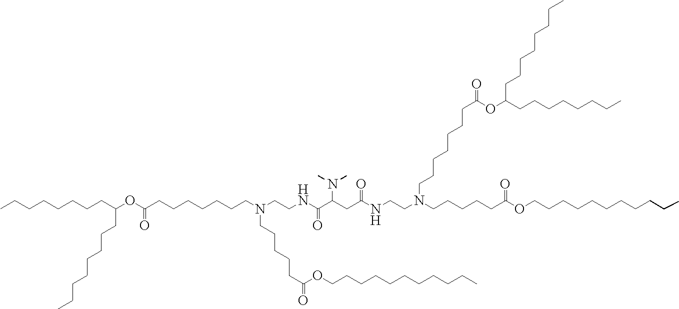 [SMILES: CCCCCCCCCCCOC(=O)CCCCCN(CCCCCCCC(=O)OC(CCCCCCCC)CCCCCCCC)CCNC(=O)CC(C(=O)NCCN(CCCCCCCC(=O)OC(CCCCCCCC)CCCCCCCC)CCCCCC(=O)OCCCCCCCCCCC)N(C)C] |
| 2285 | 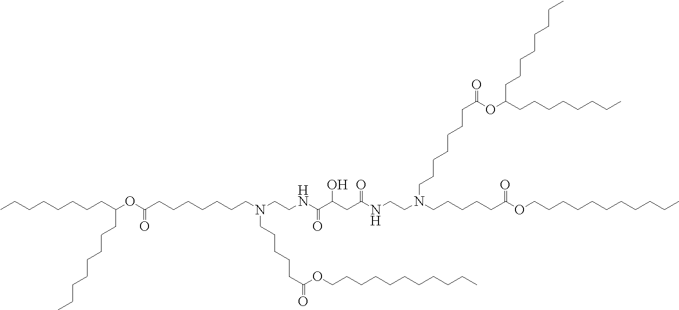 [SMILES: CCCCCCCCCCCOC(=O)CCCCCN(CCCCCCCC(=O)OC(CCCCCCCC)CCCCCCCC)CCNC(=O)CC(O)C(=O)NCCN(CCCCCCCC(=O)OC(CCCCCCCC)CCCCCCCC)CCCCCC(=O)OCCCCCCCCCCC] |
| 2284 | 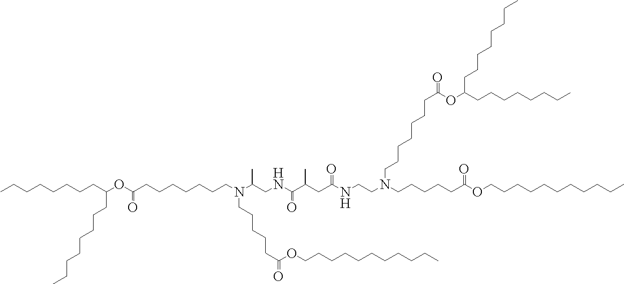 [SMILES: CCCCCCCCCCCOC(=O)CCCCCN(CCCCCCCC(=O)OC(CCCCCCCC)CCCCCCCC)CCNC(=O)CC(C)C(=O)NCC(C)N(CCCCCCCC(=O)OC(CCCCCCCC)CCCCCCCC)CCCCCC(=O)OCCCCCCCCCCC] |
| 2283 | 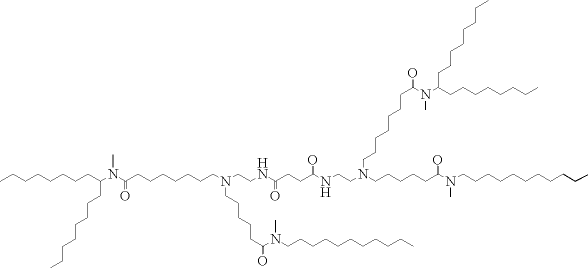 [SMILES: CCCCCCCCCCCN(C)C(=O)CCCCCN(CCCCCCCC(=O)N(C)C(CCCCCCCC)CCCCCCCC)CCNC(=O)CCC(=O)NCCN(CCCCCCCC(=O)N(C)C(CCCCCCCC)CCCCCCCC)CCCCCC(=O)N(C)CCCCCCCCCCC] |
| 2282 | 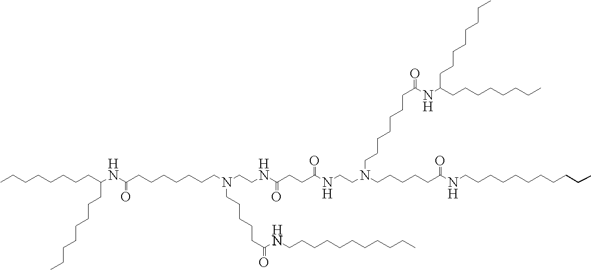 |
| 2281 | 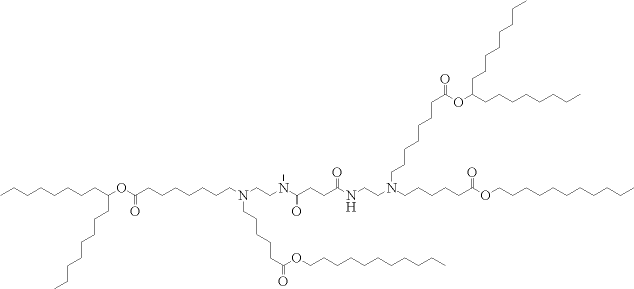 [SMILES: CCCCCCCCCCCOC(=O)CCCCCN(CCCCCCCC(=O)OC(CCCCCCCC)CCCCCCCC)CCNC(=O)CCC(=O)N(C)CCN(CCCCCCCC(=O)OC(CCCCCCCC)CCCCCCCC)CCCCCC(=O)OCCCCCCCCCCC] |
| 2280 | 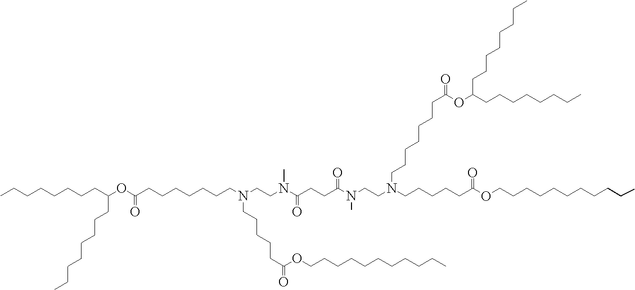 [SMILES: CCCCCCCCCCCOC(=O)CCCCCN(CCCCCCCC(=O)OC(CCCCCCCC)CCCCCCCC)CCN(C)C(=O)CCC(=O)N(C)CCN(CCCCCCCC(=O)OC(CCCCCCCC)CCCCCCCC)CCCCCC(=O)OCCCCCCCCCCC] |
| 2279 | 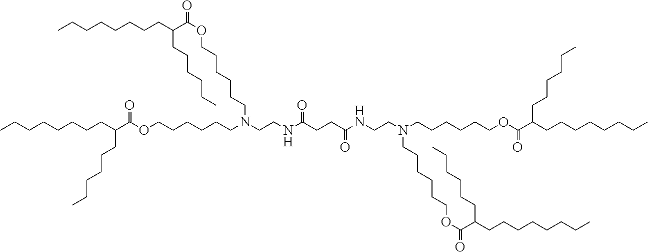 [SMILES: CCCCCCCCC(CCCCCC)C(=O)OCCCCCCN(CCCCCCOC(=O)C(CCCCCC)CCCCCCCC)CCNC(=O)CCC(=O)NCCN(CCCCCCOC(=O)C(CCCCCC)CCCCCCCC)CCCCCCOC(=O)C(CCCCCC)CCCCCCCC] |
| 2278 | 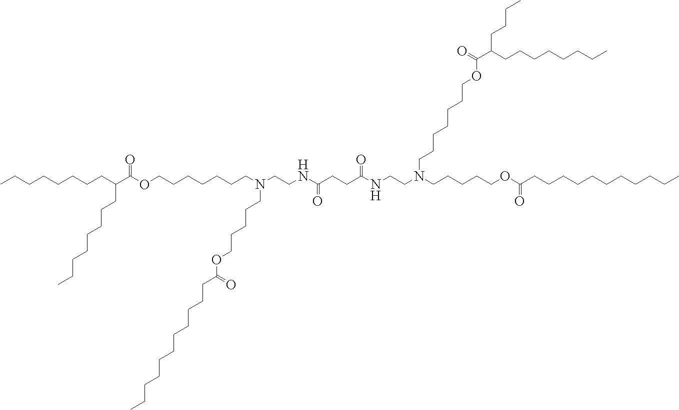 [SMILES: CCCCCCCCCCCC(=O)OCCCCCN(CCCCCCCOC(=O)C(CCCC)CCCCCCCC)CCNC(=O)CCC(=O)NCCN(CCCCCCCOC(=O)C(CCCCCCCC)CCCCCCCC)CCCCCOC(=O)CCCCCCCCCCC] |
| 2275 | 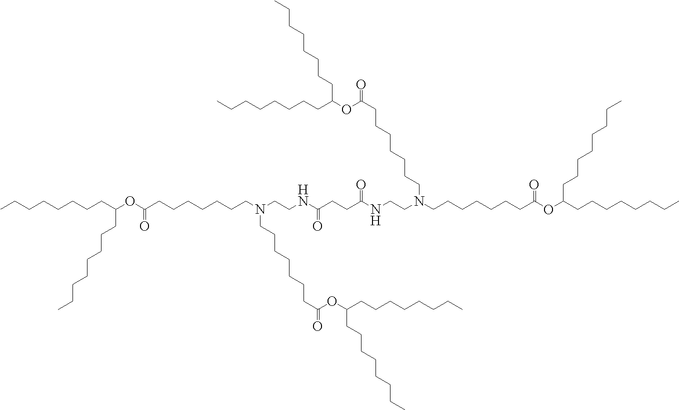 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCCCC(=O)OC(CCCCCCCC)CCCCCCCC] |
| 2274 | 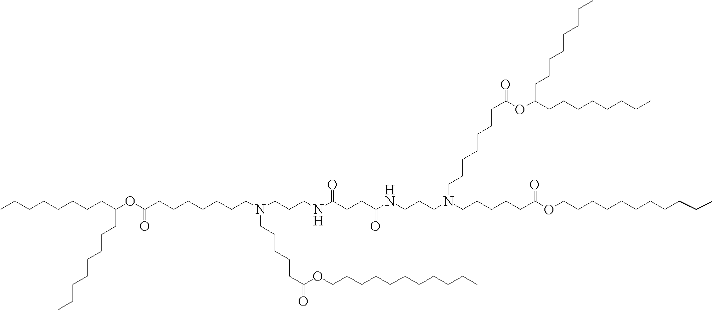 [SMILES: CCCCCCCCCCCOC(=O)CCCCCN(CCCCCCCC(=O)OC(CCCCCCCC)CCCCCCCC)CCCNC(=O)CCC(=O)NCCCN(CCCCCCCC(=O)OC(CCCCCCCC)CCCCCCCC)CCCCCC(=O)OCCCCCCCCCCC] |
| 2273 | 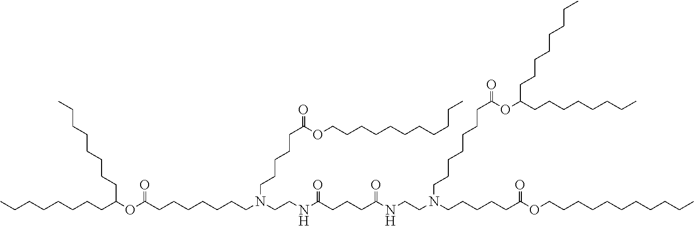 [SMILES: CCCCCCCCCCCOC(=O)CCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCC(=O)OCCCCCCCCCCC] |
| 2272 | 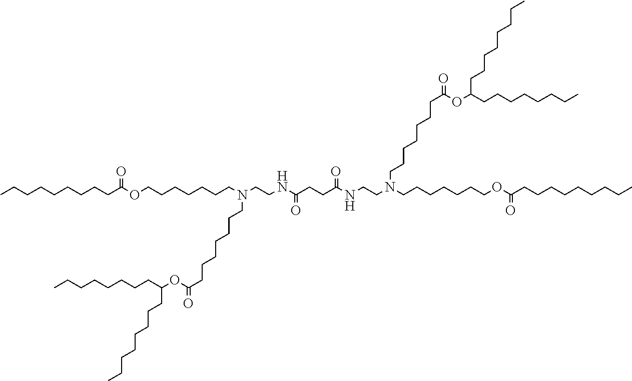 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)CCCCCCCCC)CCCCCCCC(=O)OC(CCCCCCCC)CCCCCCCC] |
| 2271 | 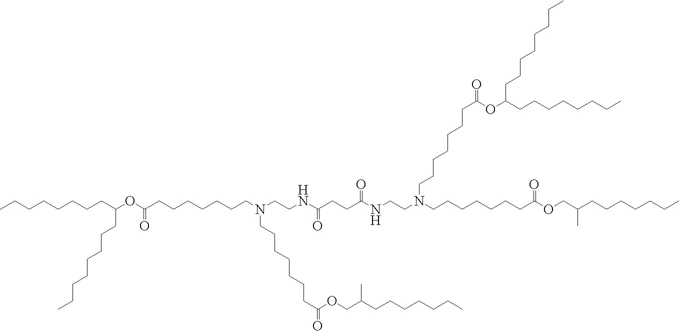 [SMILES: CCCCCCCCC(CCCCCCCC)OC(=O)CCCCCCCN(CCCCCCCC(=O)OCC(C)CCCCCCC)CCNC(=O)CCC(=O)NCCN(CCCCCCCC(=O)OCC(C)CCCCCCC)CCCCCCCC(=O)OC(CCCCCCCC)CCCCCCCC] |
| 2253 | 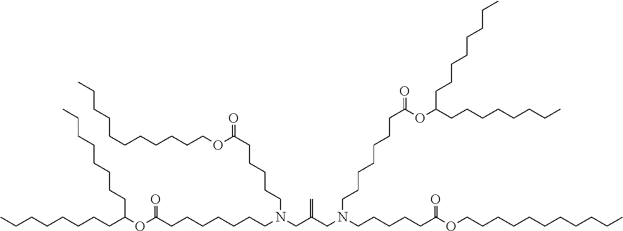 [SMILES: C=C(CN(CCCCCCCC(=O)OC(CCCCCCCC)CCCCCCCC)CCCCCC(=O)OCCCCCCCCCCC)CN(CCCCCCCC(=O)OC(CCCCCCCC)CCCCCCCC)CCCCCC(=O)OCCCCCCCCCCC] |
| 2252 | 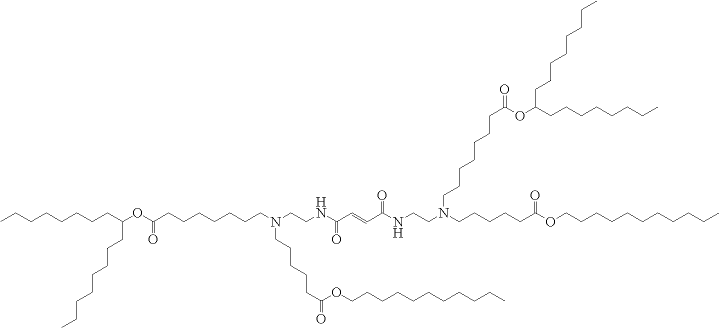 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCC(=O)OCCCCCCCCCCC] |
| 2251 | 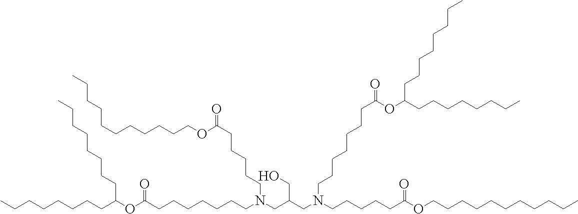 [SMILES: CCCCCCCCCCCOC(=O)CCCCCN(CCCCCCCC(=O)OC(CCCCCCCC)CCCCCCCC)CC(CO)CN(CCCCCCCC(=O)OC(CCCCCCCC)CCCCCCCC)CCCCCC(=O)OCCCCCCCCCCC] |
| 2247 | 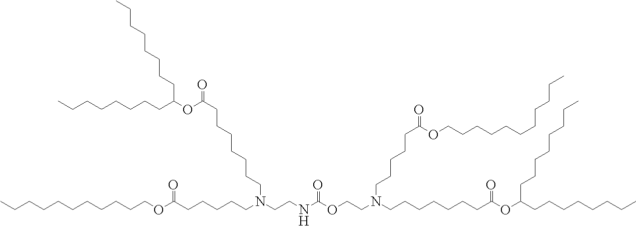 [SMILES: CCCCCCCCCCCOC(=O)CCCCCN(CCCCCCCC(=O)OC(CCCCCCCC)CCCCCCCC)CCNC(=O)OCCN(CCCCCCCC(=O)OC(CCCCCCCC)CCCCCCCC)CCCCCC(=O)OCCCCCCCCCCC] |
| 2246 | 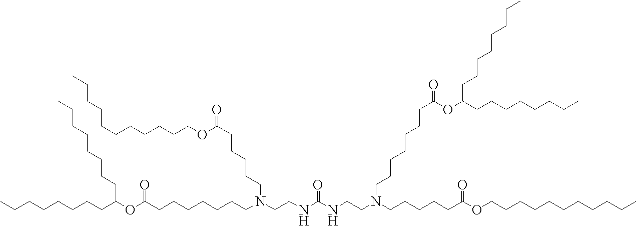 [SMILES: CCCCCCCCCCCOC(=O)CCCCCN(CCCCCCCC(=O)OC(CCCCCCCC)CCCCCCCC)CCNC(=O)NCCN(CCCCCCCC(=O)OC(CCCCCCCC)CCCCCCCC)CCCCCC(=O)OCCCCCCCCCCC] |
| 2221 | 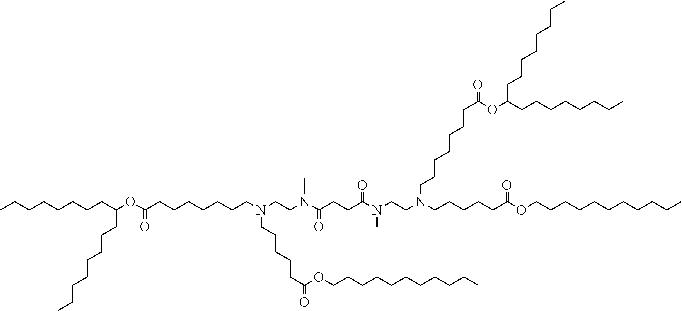 [SMILES: CCCCCCCCCCCOC(=O)CCCCCN(CCCCCCCC(=O)OC(CCCCCCCC)CCCCCCCC)CCN(C)C(=O)CCC(=O)N(C)CCN(CCCCCCCC(=O)OC(CCCCCCCC)CCCCCCCC)CCCCCC(=O)OCCCCCCCCCCC] |
| 2220 | 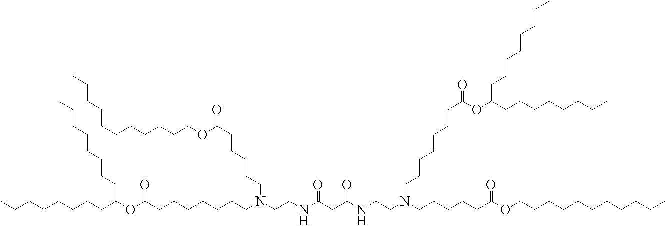 [SMILES: CCCCCCCCCCCOC(=O)CCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCC(=O)OCCCCCCCCCCC] |
| 2219 | 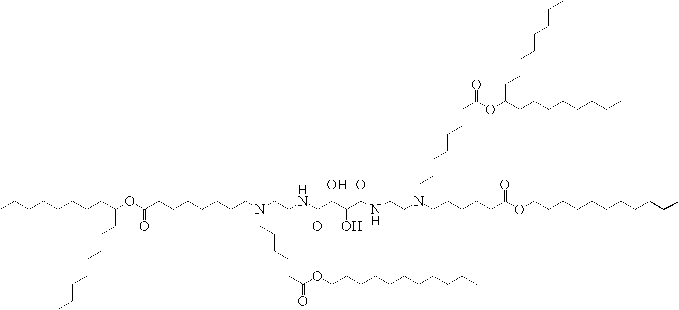 [SMILES: CCCCCCCCCCCOC(=O)CCCCCN(CCCCCCCC(=O)OC(CCCCCCCC)CCCCCCCC)CCNC(=O)C(O)C(O)C(=O)NCCN(CCCCCCCC(=O)OC(CCCCCCCC)CCCCCCCC)CCCCCC(=O)OCCCCCCCCCCC] |
| 2218 | 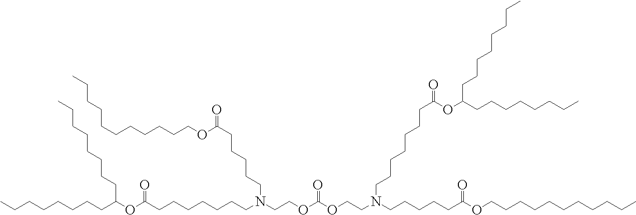 [SMILES: CCCCCCCCCCCOC(=O)CCCCCN(CCCCCCCC(=O)OC(CCCCCCCC)CCCCCCCC)CCOC(=O)OCCN(CCCCCCCC(=O)OC(CCCCCCCC)CCCCCCCC)CCCCCC(=O)OCCCCCCCCCCC] |
| 2217 | 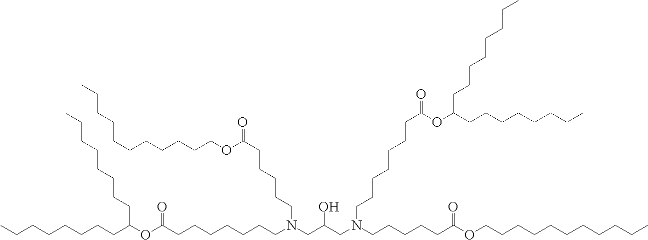 [SMILES: CCCCCCCCCCCOC(=O)CCCCCN(CCCCCCCC(=O)OC(CCCCCCCC)CCCCCCCC)CC(O)CN(CCCCCCCC(=O)OC(CCCCCCCC)CCCCCCCC)CCCCCC(=O)OCCCCCCCCCCC] |
| 2213 | 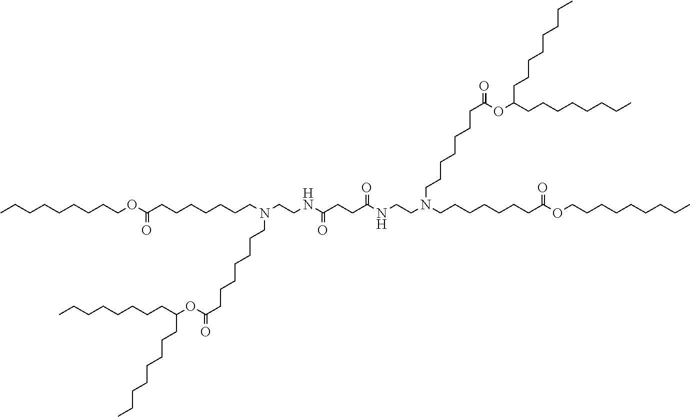 [SMILES: CCCCCCCCCOC(=O)CCCCCCCN(CCCCCCCC(=O)OC(CCCCCCCC)CCCCCCCC)CCNC(=O)CCC(=O)NCCN(CCCCCCCC(=O)OCCCCCCCCC)CCCCCCCC(=O)OC(CCCCCCCC)CCCCCCCC] |
| 2212 | 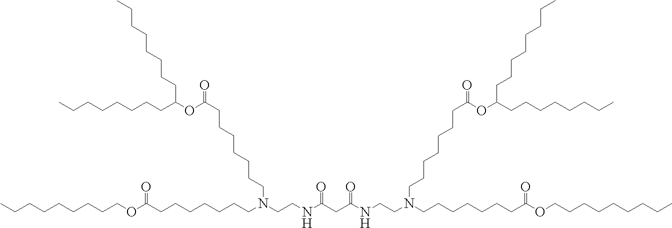 [SMILES: CCCCCCCCCOC(=O)CCCCCCCN(CCCCCCCC(=O)OC(CCCCCCCC)CCCCCCCC)CCNC(=O)CC(=O)NCCN(CCCCCCCC(=O)OCCCCCCCCC)CCCCCCCC(=O)OC(CCCCCCCC)CCCCCCCC] |
| 2211 | 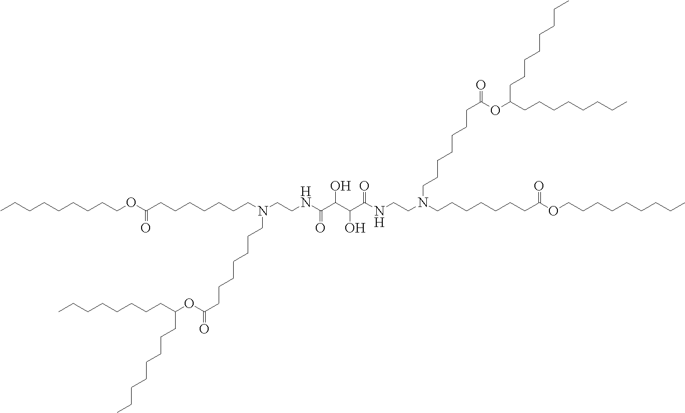 [SMILES: CCCCCCCCCOC(=O)CCCCCCCN(CCCCCCCC(=O)OC(CCCCCCCC)CCCCCCCC)CCNC(=O)C(O)C(O)C(=O)NCCN(CCCCCCCC(=O)OCCCCCCCCC)CCCCCCCC(=O)OC(CCCCCCCC)CCCCCCCC] |
| 2210 | 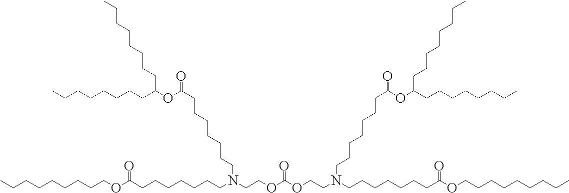 [SMILES: CCCCCCCCCOC(=O)CCCCCCCN(CCCCCCCC(=O)OC(CCCCCCCC)CCCCCCCC)CCOC(=O)OCCN(CCCCCCCC(=O)OCCCCCCCCC)CCCCCCCC(=O)OC(CCCCCCCC)CCCCCCCC] |
| 2209 | 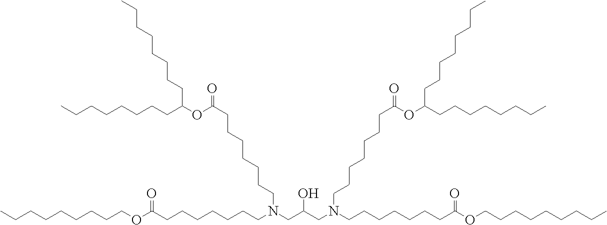 [SMILES: CCCCCCCCCOC(=O)CCCCCCCN(CCCCCCCC(=O)OC(CCCCCCCC)CCCCCCCC)CC(O)CN(CCCCCCCC(=O)OCCCCCCCCC)CCCCCCCC(=O)OC(CCCCCCCC)CCCCCCCC] |
| 2513 | 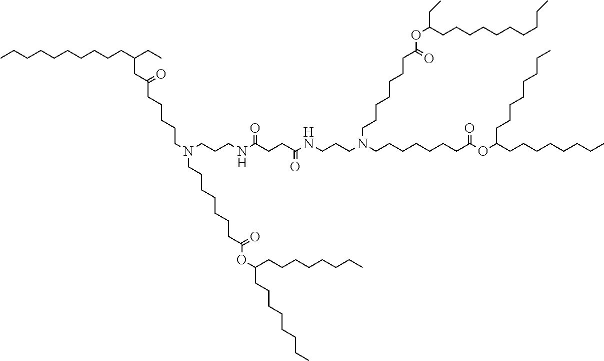 [SMILES: CCCCCCCCCCC(CC)CC(=O)CCCCCN(CCCCCCCC(=O)OC(CCCCCCCC)CCCCCCCC)CCCNC(=O)CCC(=O)NCCCN(CCCCCCCC(=O)OC(CC)CCCCCCCCCC)CCCCCCCC(=O)OC(CCCCCCCC)CCCCCCCC] |
| | 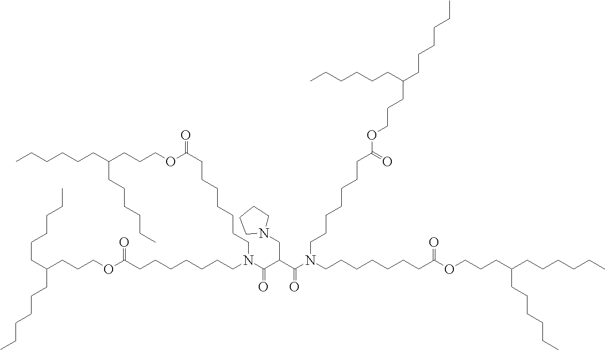 [SMILES: CCCCCCC(CCCCCC)CCCOC(=O)CCCCCCCN(CCCCCCCC(=O)OCCCC(CCCCCC)CCCCCC)C(=O)C(CN1CCCC1)C(=O)N(CCCCCCCC(=O)OCCCC(CCCCCC)CCCCCC)CCCCCCCC(=O)OCCCC(CCCCCC)CCCCCC] |
| | 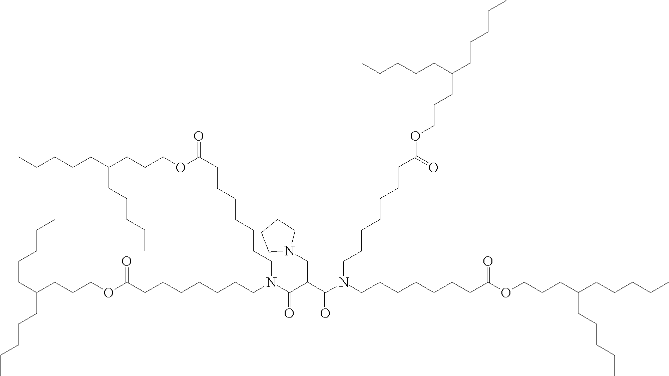 [SMILES: CCCCCC(CCCCC)CCCOC(=O)CCCCCCCN(CCCCCCCC(=O)OCCCC(CCCCC)CCCCC)C(=O)C(CN1CCCC1)C(=O)N(CCCCCCCC(=O)OCCCC(CCCCC)CCCCC)CCCCCCCC(=O)OCCCC(CCCCC)CCCCC] |
| | 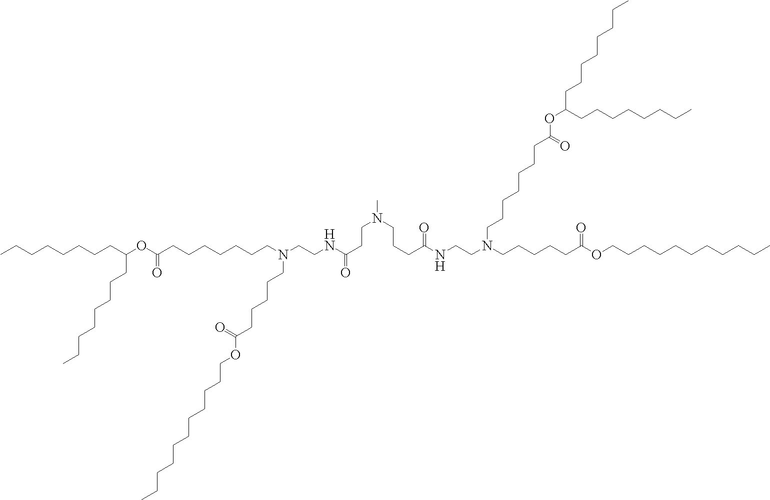 [SMILES: CCCCCCCCCCCOC(=O)CCCCCN(CCCCCCCC(=O)OC(CCCCCCCC)CCCCCCCC)CCNC(=O)CCCN(C)CCC(=O)NCCN(CCCCCCCC(=O)OC(CCCCCCCC)CCCCCCCC)CCCCCC(=O)OCCCCCCCCCCC] |
| | 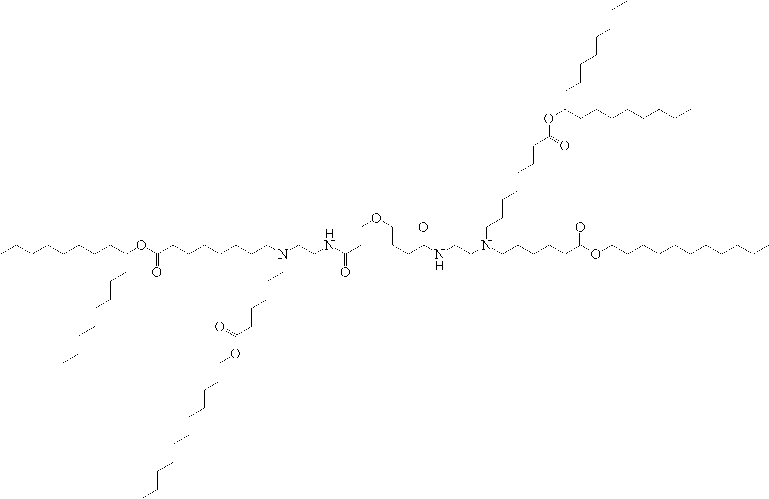 [SMILES: CCCCCCCCCCCOC(=O)CCCCCN(CCCCCCCC(=O)OC(CCCCCCCC)CCCCCCCC)CCNC(=O)CCCOCCC(=O)NCCN(CCCCCCCC(=O)OC(CCCCCCCC)CCCCCCCC)CCCCCC(=O)OCCCCCCCCCCC] |
| 2248 | 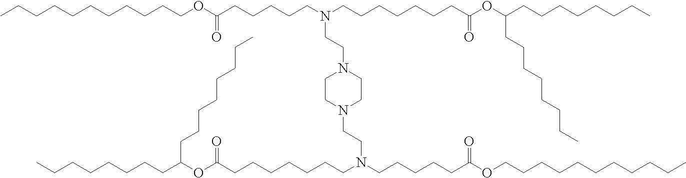 [SMILES: CCCCCCCCCCCOC(=O)CCCCCN(CCCCCCCC(=O)OC(CCCCCCCC)CCCCCCCC)CCN1CCN(CCN(CCCCCCCC(=O)OC(CCCCCCCC)CCCCCCCC)CCCCCC(=O)OCCCCCCCCCCC)CC1] |
| 2190 | 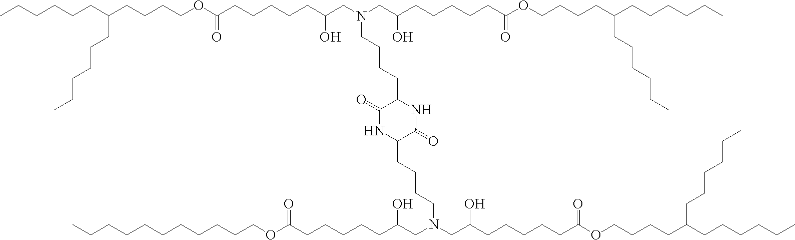 [SMILES: CCCCCCCCCCCOC(=O)CCCCCC(O)CN(CCCCC1NC(=O)C(CCCCN(CC(O)CCCCCC(=O)OCCCCC(CCCCCC)CCCCCC)CC(O)CCCCCC(=O)OCCCCC(CCCCCC)CCCCCC)NC1=O)CC(O)CCCCCC(=O)OCCCCC(CCCCCC)CCCCCC] |
| 2189 | 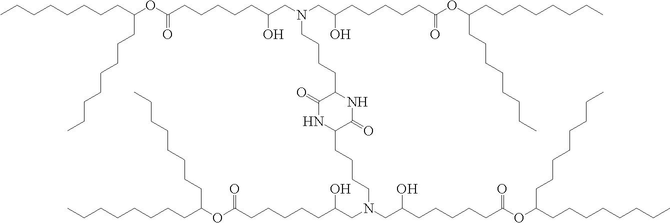 |
| 2287 | 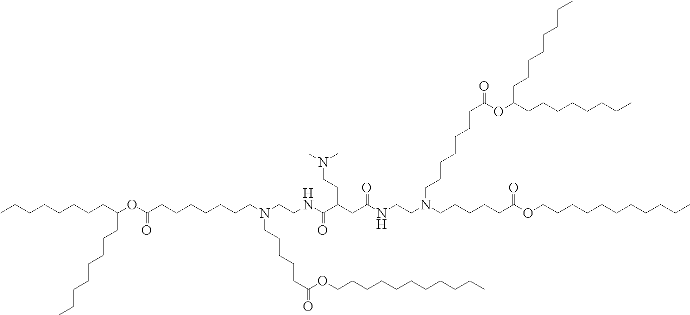 [SMILES: CCCCCCCCCCCOC(=O)CCCCCN(CCCCCCCC(=O)OC(CCCCCCCC)CCCCCCCC)CCNC(=O)CC(CCN(C)C)C(=O)NCCN(CCCCCCCC(=O)OC(CCCCCCCC)CCCCCCCC)CCCCCC(=O)OCCCCCCCCCCC] |
| 2315 | 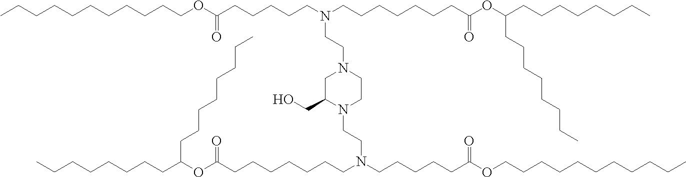 [SMILES: CCCCCCCCCCCOC(=O)CCCCCN(CCCCCCCC(=O)OC(CCCCCCCC)CCCCCCCC)CCN1CCN(CCN(CCCCCCCC(=O)OC(CCCCCCCC)CCCCCCCC)CCCCCC(=O)OCCCCCCCCCCC)[C@@H](CO)C1] |
| 2316 | 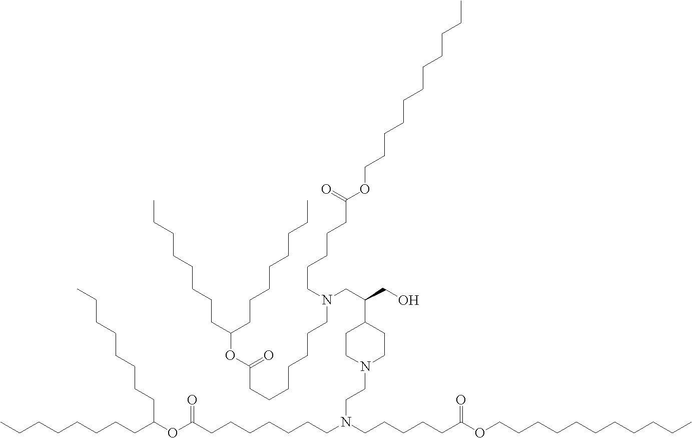 [SMILES: CCCCCCCCCCCOC(=O)CCCCCN(CCCCCCCC(=O)OC(CCCCCCCC)CCCCCCCC)CCN1CCC([C@H](CO)CN(CCCCCCCC(=O)OC(CCCCCCCC)CCCCCCCC)CCCCCC(=O)OCCCCCCCCCCC)CC1] |
| 2317 | 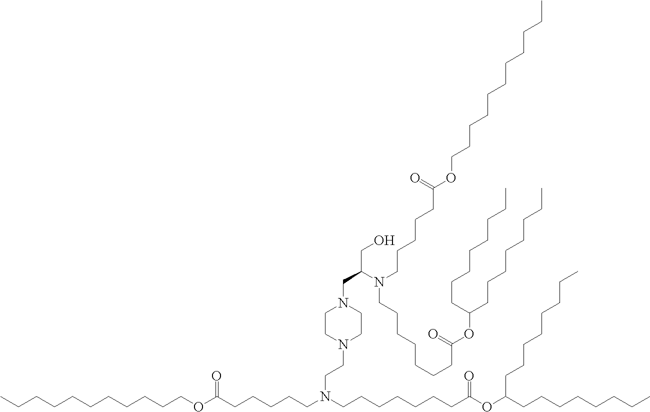 [SMILES: CCCCCCCCCCCOC(=O)CCCCCN(CCCCCCCC(=O)OC(CCCCCCCC)CCCCCCCC)CCN1CCN(C[C@@H](CO)N(CCCCCCCC(=O)OC(CCCCCCCC)CCCCCCCC)CCCCCC(=O)OCCCCCCCCCCC)CC1] |
| 2318 | 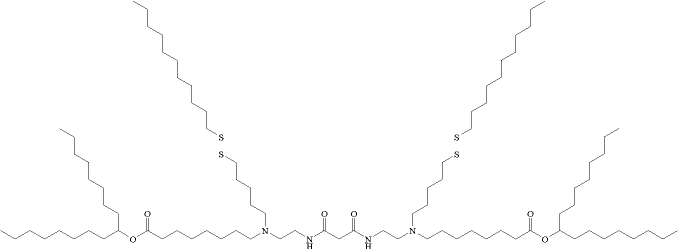 |
| 2439 | 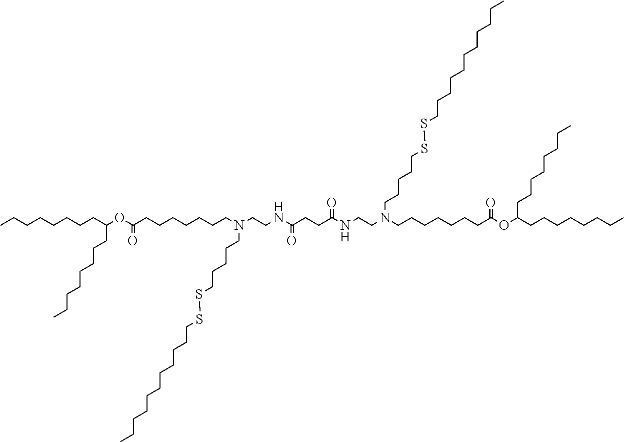 |
| 2319 | 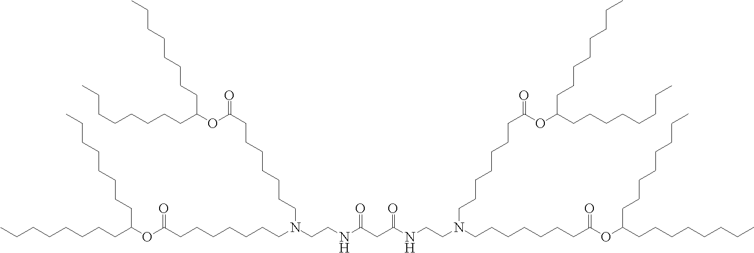 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCCCC(=O)OC(CCCCCCCC)CCCCCCCC] |
| 2320 | 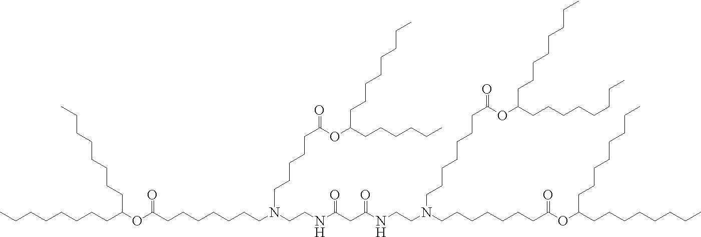 |
| 2321 | 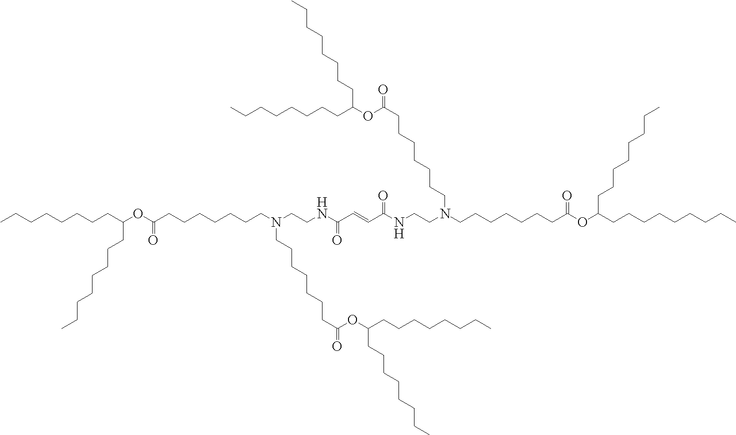 [SMILES: CCCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCCCC(=O)OC(CCCCCCCC)CCCCCCCC] |
| 2322 | 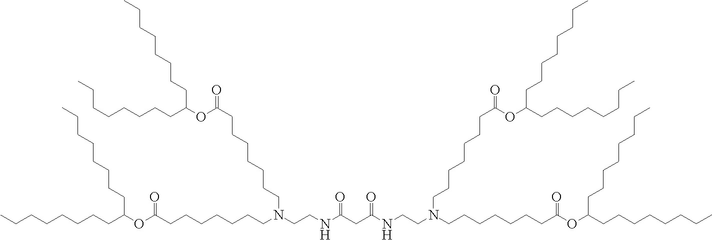 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCCCC(=O)OC(CCCCCCCC)CCCCCCCC] |
| 2323 | 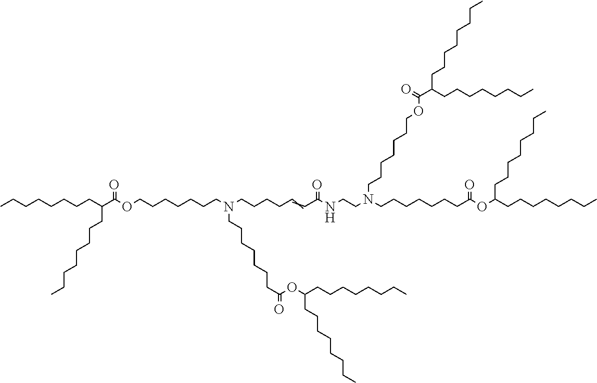 [SMILES: CCCCCCCCC(CCCCCCCC)OC(=O)CCCCCCCN(CCCCC=CC(=O)NCCN(CCCCCCCOC(=O)C(CCCCCCCC)CCCCCCCC)CCCCCCCC(=O)OC(CCCCCCCC)CCCCCCCC)CCCCCCCOC(=O)C(CCCCCCCC)CCCCCCCC] |
| 2324 | 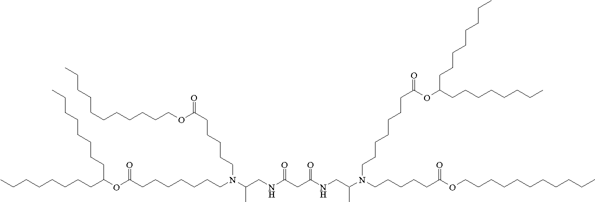 [SMILES: CCCCCCCCCCCOC(=O)CCCCCN(CCCCCCCC(=O)OC(CCCCCCCC)CCCCCCCC)C(C)CNC(=O)CC(=O)NCC(C)N(CCCCCCCC(=O)OC(CCCCCCCC)CCCCCCCC)CCCCCC(=O)OCCCCCCCCCCC] |
| 2325 | 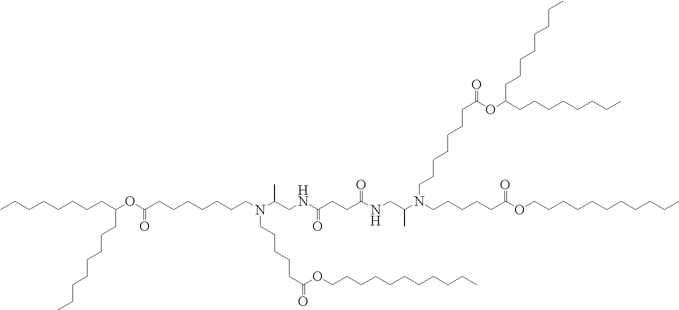 [SMILES: CCCCCCCCCCCOC(=O)CCCCCN(CCCCCCCC(=O)OC(CCCCCCCC)CCCCCCCC)C(C)CNC(=O)CCC(=O)NCC(C)N(CCCCCCCC(=O)OC(CCCCCCCC)CCCCCCCC)CCCCCC(=O)OCCCCCCCCCCC] |
| 2326 | 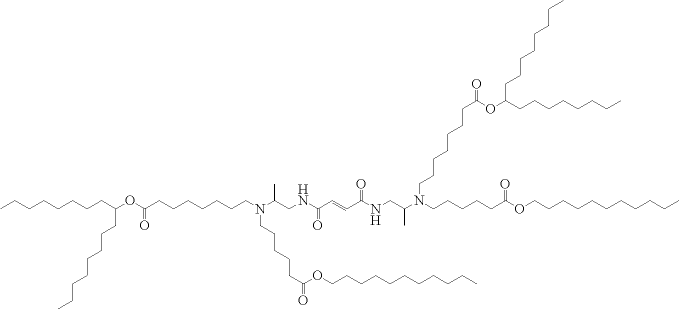 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCCC(=O)OCCCCCCCCCCC] |
| 2327 | 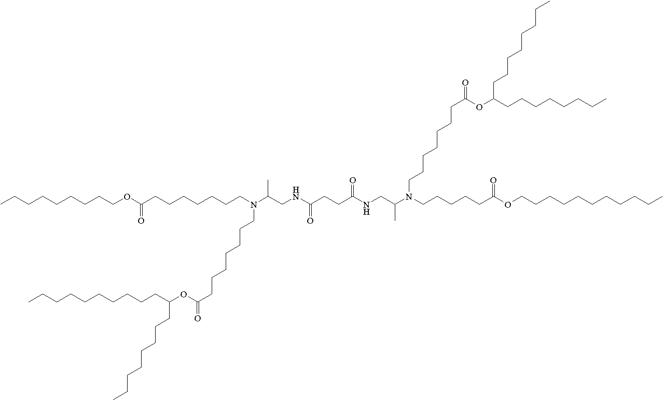 [SMILES: CCCCCCCCCCCOC(=O)CCCCCN(CCCCCCCC(=O)OC(CCCCCCCC)CCCCCCCC)C(C)CNC(=O)CCC(=O)NCC(C)N(CCCCCCCC(=O)OCCCCCCCCC)CCCCCCCC(=O)OC(CCCCCCCC)CCCCCCCCCC] |
| 2328 | 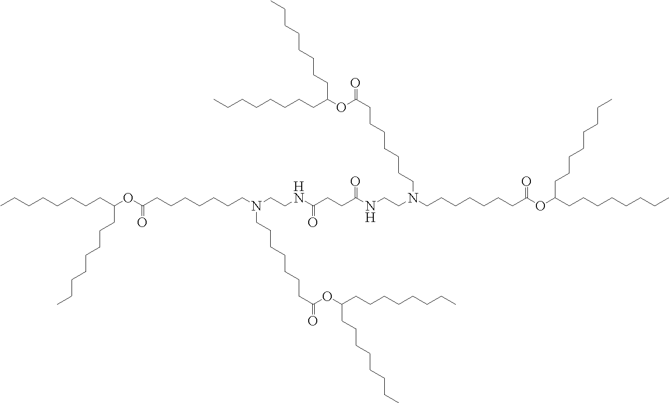 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCCCC(=O)OC(CCCCCCCC)CCCCCCCC] |
| 2332 | 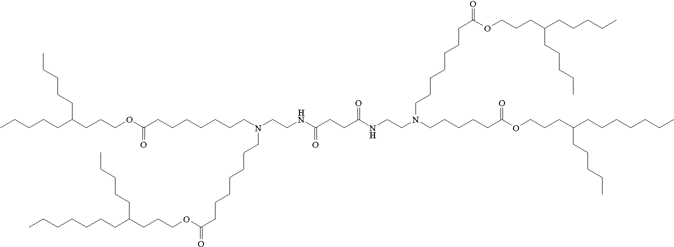 [SMILES: CCCCCCCC(CCCCC)CCCOC(=O)CCCCCCCN(CCCCCCCC(=O)OCCCC(CCCCC)CCCCC)CCNC(=O)CCC(=O)NCCN(CCCCCCCC(=O)OCCCC(CCCCC)CCCCC)CCCCCC(=O)OCCCC(CCCCC)CCCCCCC] |
| 2334 | 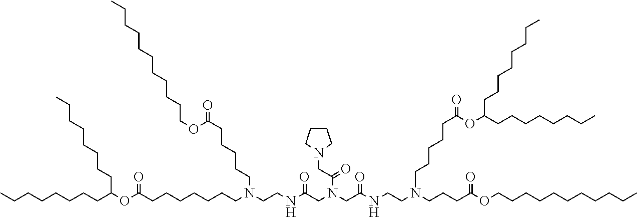 |
| 2353 | 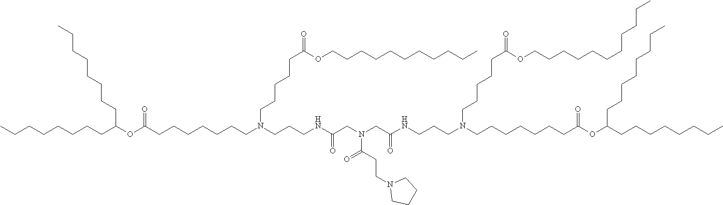 [SMILES: CCCCCCCCCCCOC(=O)CCCCCN(CCCCCCCC(=O)OC(CCCCCCCC)CCCCCCCC)CCCNC(=O)CN(CC(=O)NCCCN(CCCCCCCC(=O)OC(CCCCCCCC)CCCCCCCC)CCCCCC(=O)OCCCCCCCCCCC)C(=O)CCN1CCCC1] |
| 2354 | 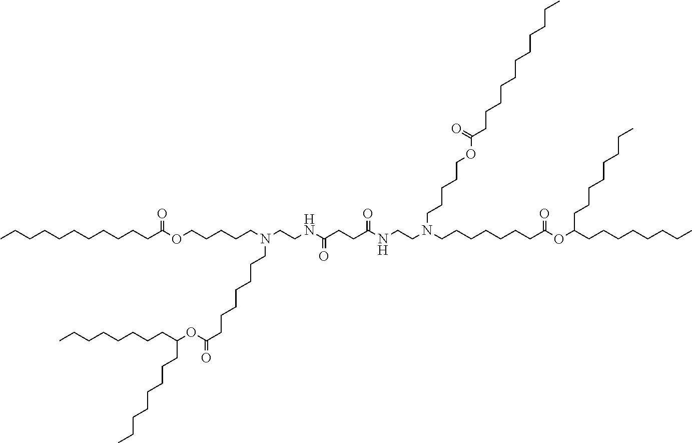 [SMILES: CCCCCCCCCCCC(=O)OCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCOC(=O)CCCCCCCCCCC] |
| 2355 | 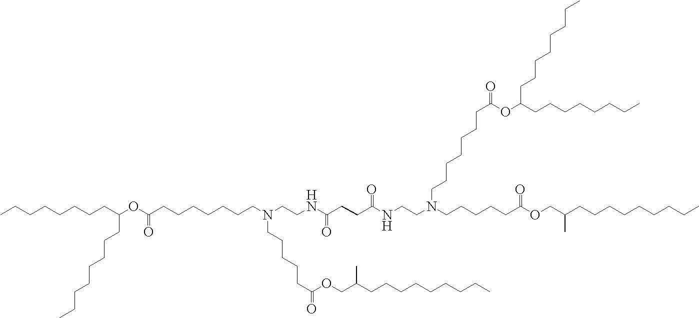 [SMILES: CCCCCCCCCC(C)COC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCC(=O)OCC(C)CCCCCCCCC] |
| 2356 | 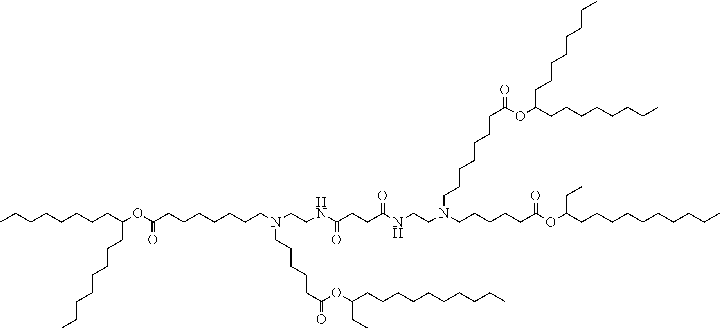 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCC(=O)OC(CC)CCCCCCCCCC] |
| 2357 | 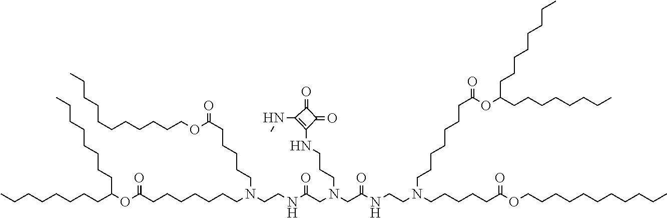 [SMILES: CCCCCCCCCCCOC(=O)CCCCCN(CCCCCCCC(=O)OC(CCCCCCCC)CCCCCCCC)CCNC(=O)CN(CCCNc1c(NC)c(=O)c1=O)CC(=O)NCCN(CCCCCCCC(=O)OC(CCCCCCCC)CCCCCCCC)CCCCCC(=O)OCCCCCCCCCCC] |
| 2358 | 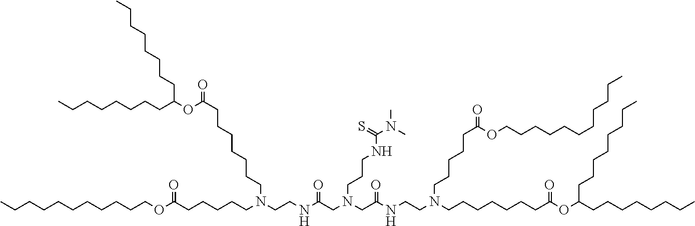 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCCC(=O)OCCCCCCCCCCC] |
| 2361 | 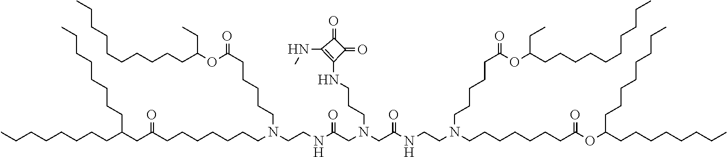 [SMILES: CCCCCCCCCCC(CC)OC(=O)CCCCCN(CCCCCCCC(=O)CC(CCCCCCCC)CCCCCCCC)CCNC(=O)CN(CCCNc1c(NC)c(=O)c1=O)CC(=O)NCCN(CCCCCCCC(=O)OC(CCCCCCCC)CCCCCCCC)CCCCCC(=O)OC(CC)CCCCCCCCCC] |
| 2362 | 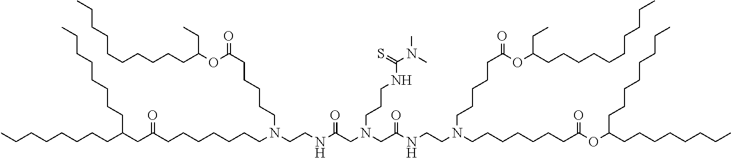 [SMILES: CCCCCCCCCCC(CC)OC(=O)CCCCCN(CCCCCCCC(=O)CC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCCC(=O)OC(CC)CCCCCCCCCC] |
| 2378 | 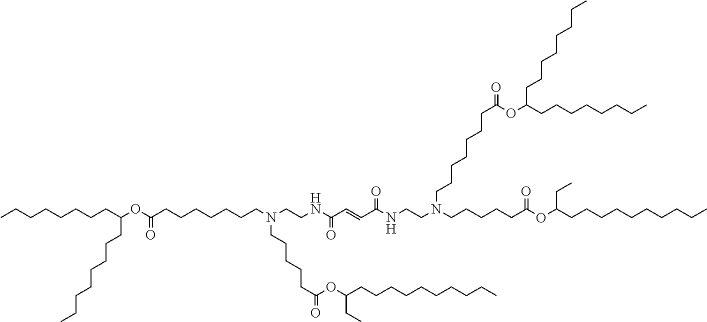 [SMILES: CCCCCCCCCCC(CC)OC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCC(=O)OC(CC)CCCCCCCCCC] |
| 2379 | 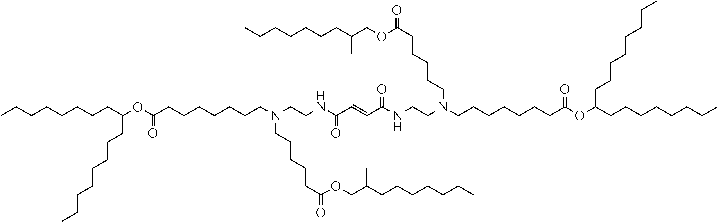 [SMILES: CCCCCCCCC(CCCCCCCC)OC(=O)CCCCCCCN(CCCCCC(=O)OCC(C)CCCCCCC)CCNC(=O)/C=C/C(=O)NCCN(CCCCCCCC(=O)OC(CCCCCCCC)CCCCCCCC)CCCCCC(=O)OCC(C)CCCCCCC] |
| 2380 | 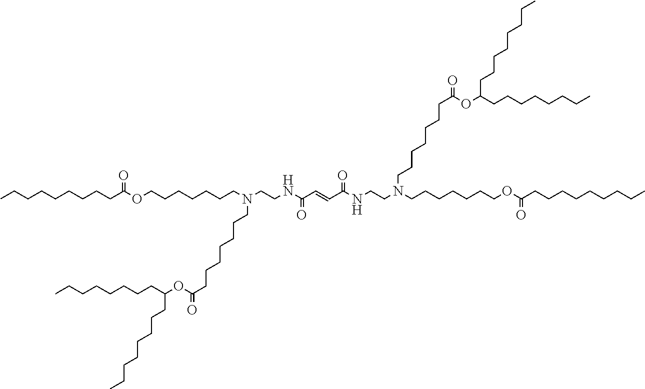 [SMILES: CCCCCCCCCC(=O)OCCCCCCCN(CCCCCCCC(=O)OC(CCCCCCCC)CCCCCCCC)CCNC(=O)/C=C/C(=O)NCCN(CCCCCCCOC(=O)CCCCCCCCC)CCCCCCCC(=O)OC(CCCCCCCC)CCCCCCCC] |
| 2381 | 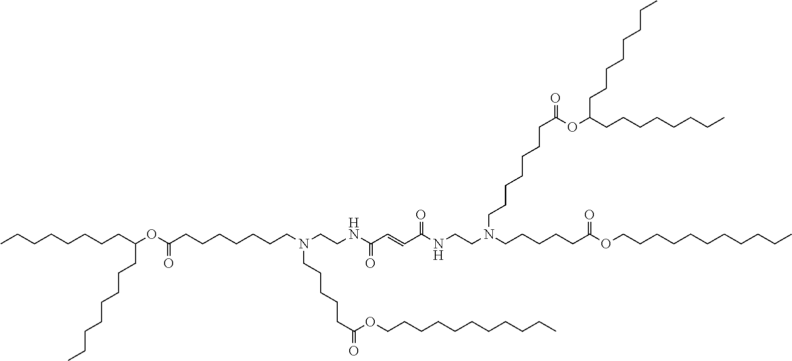 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCC(=O)OCCCCCCCCCCC] |
| 2382 | 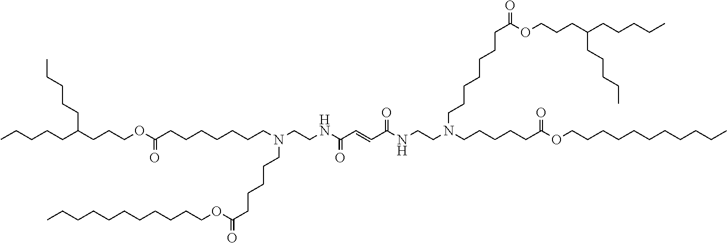 [SMILES: CCCCCCCCCCCOC(=O)CCCCCN(CCCCCCCC(=O)OCCCC(CCCCC)CCCCC)CCNC(=O)/C=C/C(=O)NCCN(CCCCCCCC(=O)OCCCC(CCCCC)CCCCC)CCCCCC(=O)OCCCCCCCCCCC] |
| 2384 | 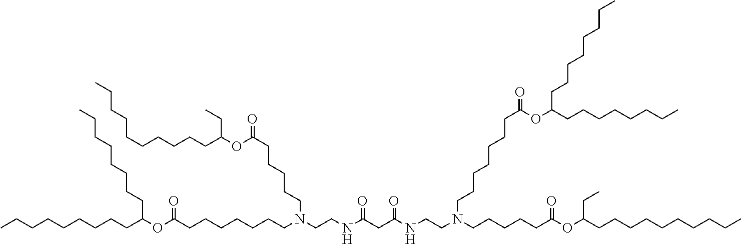 [SMILES: CCCCCCCCCCC(CC)OC(=O)CCCCCN(CCCCCCCC(=O)OC(CCCCCCCC)CCCCCCCC)CCNC(=O)CC(=O)NCCN(CCCCCCCC(=O)OC(CCCCCCCC)CCCCCCCCC)CCCCCC(=O)OC(CC)CCCCCCCCCC] |
| 2385 | 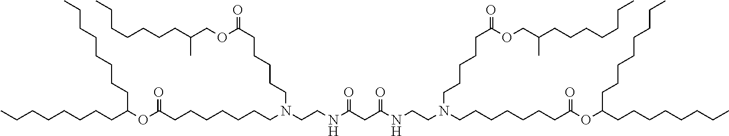 [SMILES: CCCCCCCCC(CCCCCCCC)OC(=O)CCCCCCCN(CCCCCC(=O)OCC(C)CCCCCCC)CCNC(=O)CC(=O)NCCN(CCCCCCCC(=O)OC(CCCCCCCC)CCCCCCCC)CCCCCC(=O)OCC(C)CCCCCCC] |
| 2386 | 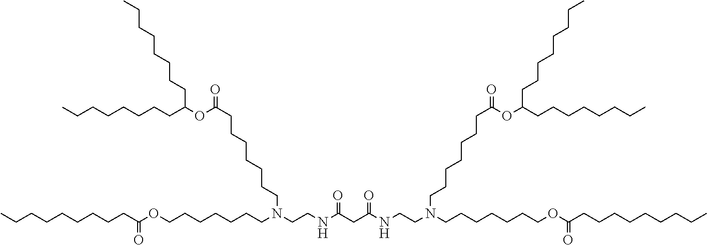 [SMILES: CCCCCCCCCC(=O)OCCCCCCCN(CCCCCCCC(=O)OC(CCCCCCCC)CCCCCCCC)CCNC(=O)CC(=O)NCCN(CCCCCCCOC(=O)CCCCCCCCC)CCCCCCCC(=O)OC(CCCCCCCC)CCCCCCCC] |
| 2387 | 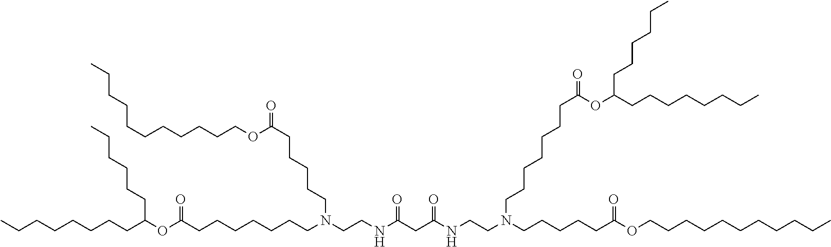 [SMILES: CCCCCCCCCCCOC(=O)CCCCCN(CCCCCCCC(=O)OC(CCCCCC)CCCCCCCC)CCNC(=O)CC(=O)NCCN(CCCCCCCC(=O)OC(CCCCCC)CCCCCCCC)CCCCCC(=O)OCCCCCCCCCCC] |
| 2388 | 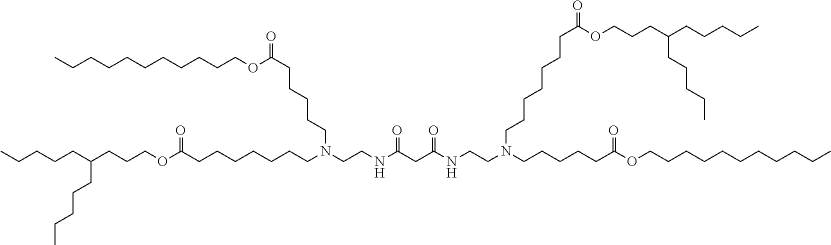 [SMILES: CCCCCCCCCCCOC(=O)CCCCCN(CCCCCCCC(=O)OCCCC(CCCCC)CCCCC)CCNC(=O)CC(=O)NCCN(CCCCCCCC(=O)OCCCC(CCCCC)CCCCC)CCCCCC(=O)OCCCCCCCCCCC] |
| 2389 | 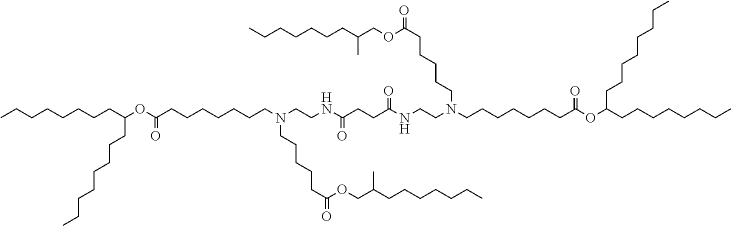 [SMILES: CCCCCCCCC(CCCCCCCC)OC(=O)CCCCCCCN(CCCCCC(=O)OCC(C)CCCCCCC)CCNC(=O)CCC(=O)NCCN(CCCCCCCC(=O)OC(CCCCCCCC)CCCCCCCC)CCCCCC(=O)OCC(C)CCCCCCC] |
| 2390 | 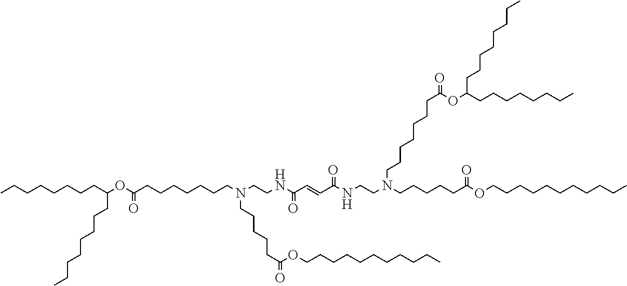 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCC(=O)OCCCCCCCCCCC] |
| 2391 | 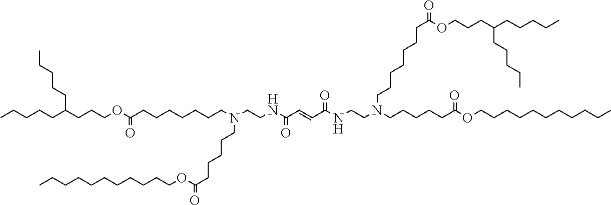 [SMILES: CCCCCCCCCCCOC(=O)CCCCCN(CCCCCCCC(=O)OCCCC(CCCCC)CCCCC)CCNC(=O)/C=C/C(=O)NCCN(CCCCCCCC(=O)OCCCC(CCCCC)CCCCC)CCCCCC(=O)OCCCCCCCCCCC] |
| 2393 | 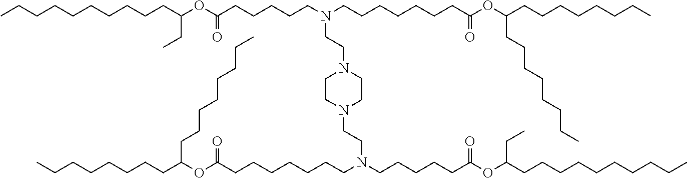 |
| 2394 | 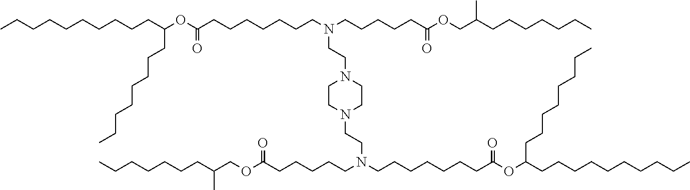 |
| 2395 | 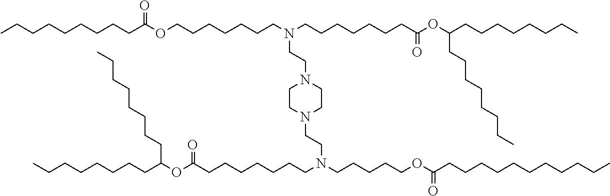 |
| 2396 | 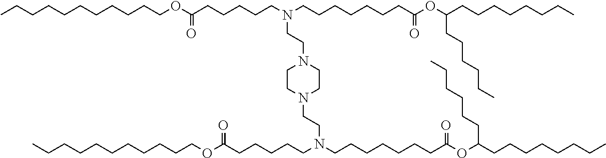 |
| 2397 | 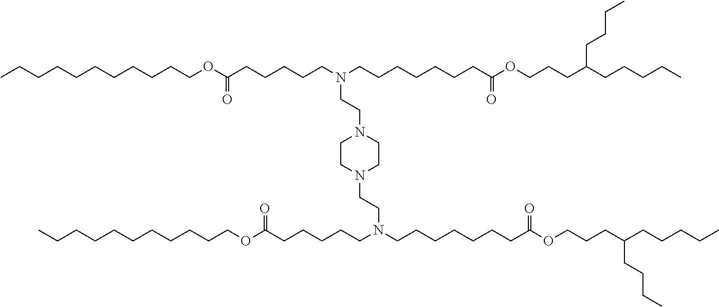 |
| 2399 | 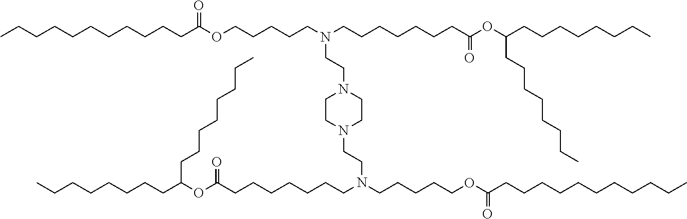 |
| 2400 | 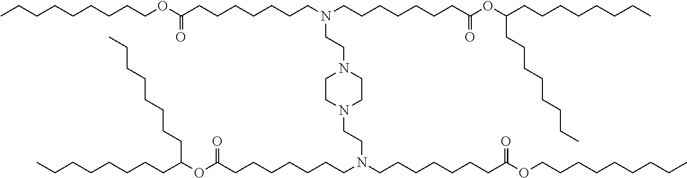 |
| 2401 | 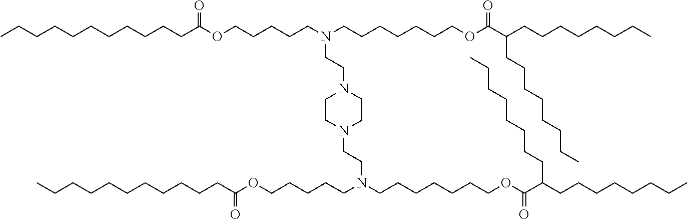 |
| 2403 | 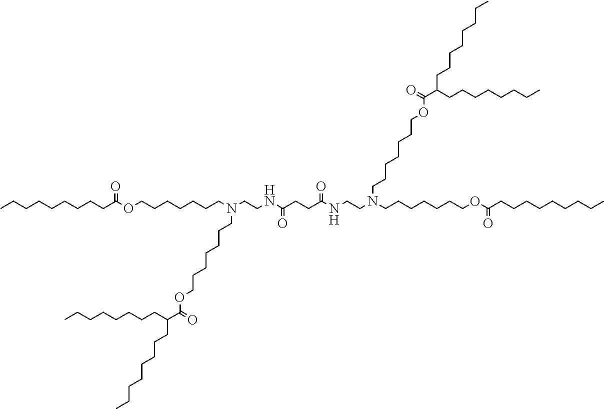 [SMILES: CCCCCCCCCC(=O)OCCCCCCCN(CCCCCCCOC(=O)C(CCCCCCCC)CCCCCCCC)CCNC(=O)CCC(=O)NCCN(CCCCCCCOC(=O)CCCCCCCCC)CCCCCCCOC(=O)C(CCCCCCCC)CCCCCCCC] |
| 2404 | 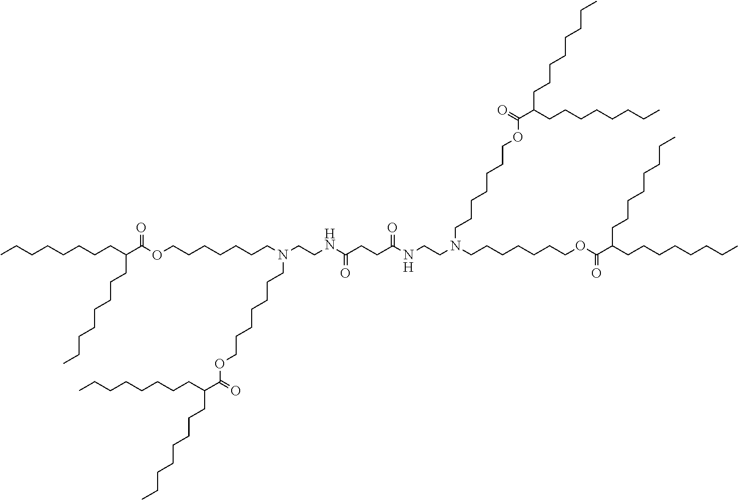 [SMILES: CCCCCCCCC(CCCCCCCC)C(=O)OCCCCCCCN(CCCCCCCOC(=O)C(CCCCCCCC)CCCCCCCC)CCNC(=O)CCC(=O)NCCN(CCCCCCCOC(=O)C(CCCCCCCC)CCCCCCCC)CCCCCCCOC(=O)C(CCCCCCCC)CCCCCCCC] |
| 2405 | 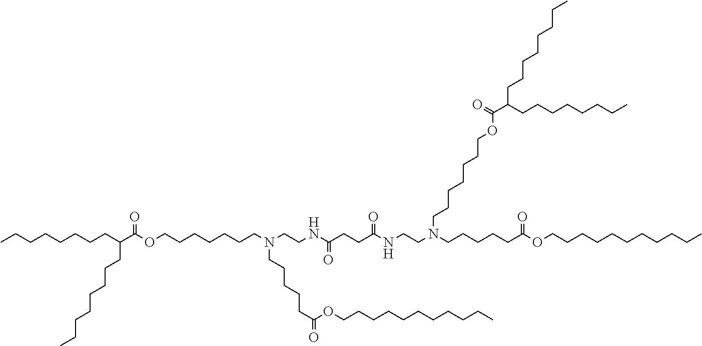 [SMILES: CCCCCCCCCCCOC(=O)CCCCCN(CCCCCCCOC(=O)C(CCCCCCCC)CCCCCCCC)CCNC(=O)CCC(=O)NCCN(CCCCCCCOC(=O)C(CCCCCCCC)CCCCCCCC)CCCCCC(=O)OCCCCCCCCCCC] |
| 2421 | 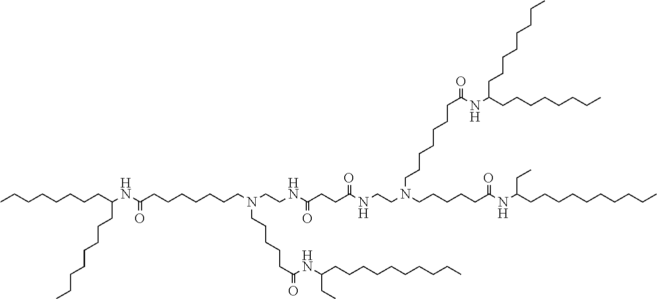 [SMILES: CCCCCCCCCCC(CC)NC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCCC(=O)NC(CC)CCCCCCCCCC] |
| 2426 | 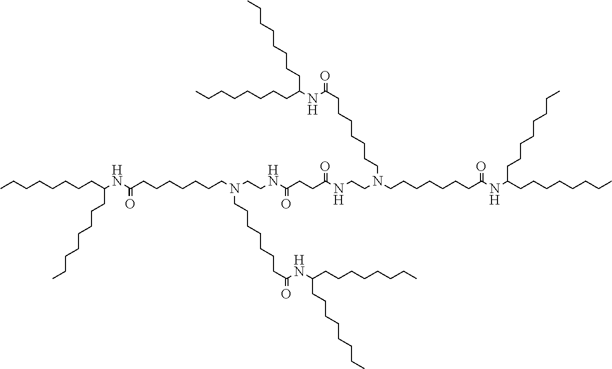 [SMILES: CCCCCCCCC(CCCCCCCC)NC(=O)CCCCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCCCCC(=O)NC(CCCCCCCC)CCCCCCCC] |
| 2427 | 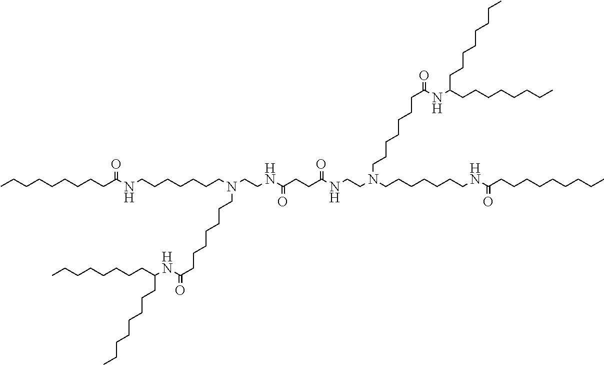 [SMILES: CCCCCCCCCC(=O)NCCCCCCCN(CCCCCCCC(=O)NC(CCCCCCCC)CCCCCCCC)CCNC(=O)CCC(=O)NCCN(CCCCCCCNC(=O)CCCCCCCCC)CCCCCCCC(=O)NC(CCCCCCCC)CCCCCCCC] |
| 2428 | 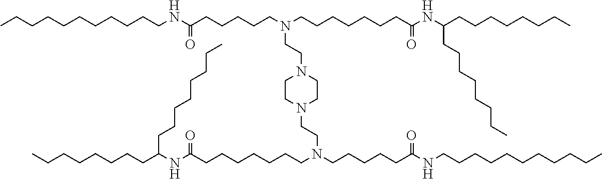 |
| | 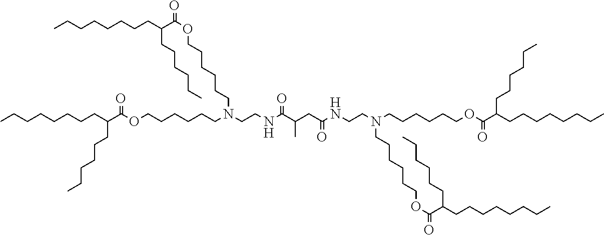 [SMILES: CCCCCCCCC(CCCCCC)C(=O)OCCCCCCN(CCCCCCOC(=O)C(CCCCCC)CCCCCCCC)CCNC(=O)CC(C)C(=O)NCCN(CCCCCCOC(=O)C(CCCCCC)CCCCCCCC)CCCCCCOC(=O)C(CCCCCC)CCCCCCCC] |
| | 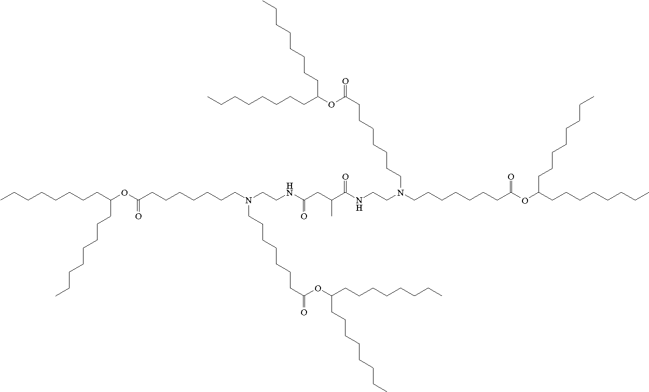 [SMILES: CCCCCCCCC(CCCCCCCC)OC(=O)CCCCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCCCC(=O)OC(CCCCCCCC)CCCCCCCC] |
| | 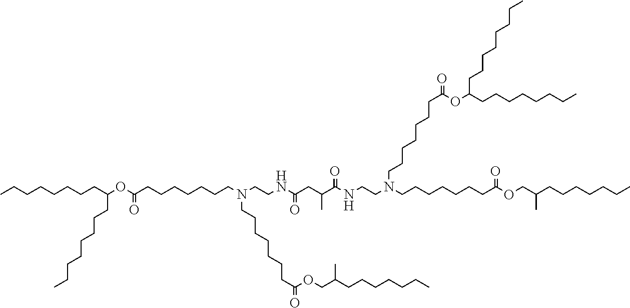 [SMILES: CCCCCCCCC(CCCCCCCC)OC(=O)CCCCCCCN(CCCCCCCC(=O)OCC(C)CCCCCCC)CCNC(=O)CC(C)C(=O)NCCN(CCCCCCCC(=O)OCC(C)CCCCCCC)CCCCCCCC(=O)OC(CCCCCCCC)CCCCCCCC] |
| | 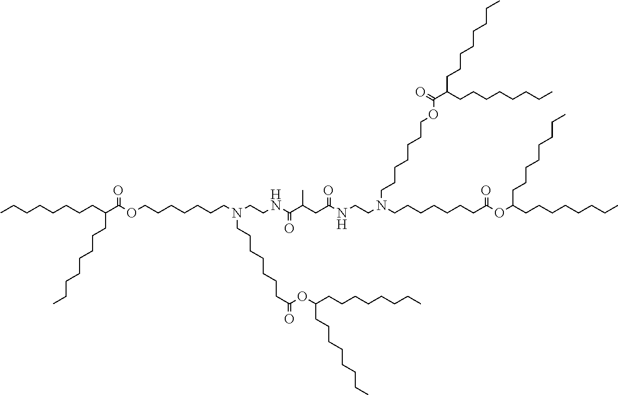 [SMILES: CCCCCCCCC(CCCCCCCC)OC(=O)CCCCCCCN(CCCCCCCOC(=O)C(CCCCCCCC)CCCCCCCC)CCNC(=O)CC(C)C(=O)NCCN(CCCCCCCOC(=O)C(CCCCCCCC)CCCCCCCC)CCCCCCCC(=O)OC(CCCCCCCC)CCCCCCCC] |
| | 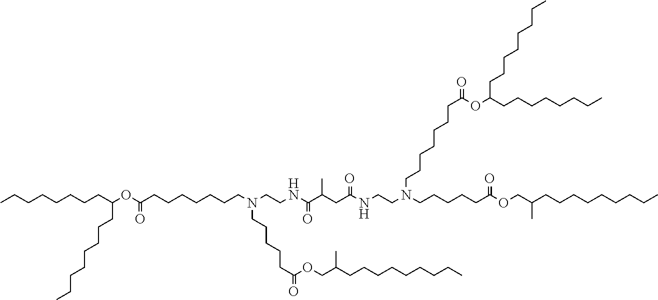 [SMILES: CCCCCCCCCC(C)COC(=O)CCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCC(=O)OCC(C)CCCCCCCCC] |
| | 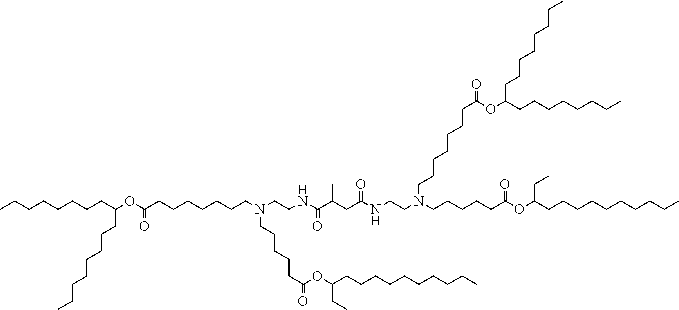 [SMILES: CCCCCCCCCCC(CC)OC(=O)CCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCC(=O)OC(CC)CCCCCCCCCC] |
| | 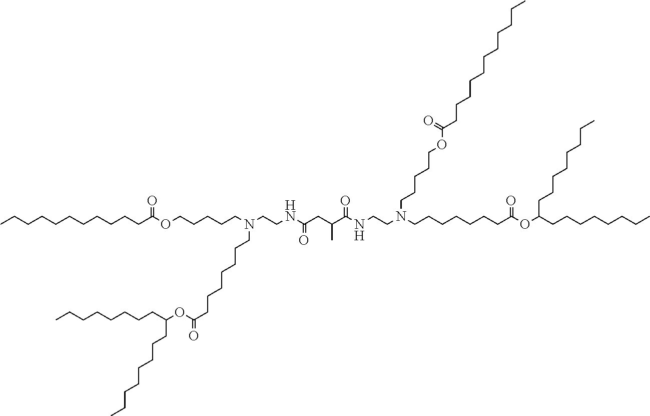 [SMILES: CCCCCCCCCCCC(=O)OCCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCOC(=O)CCCCCCCCCCC] |
| | 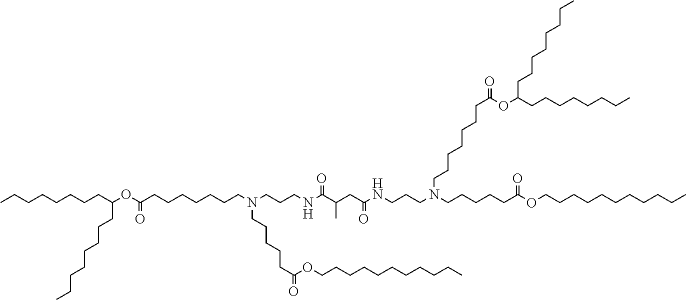 [SMILES: CCCCCCCCCCCOC(=O)CCCCCN(CCCCCCCC(=O)OC(CCCCCCCC)CCCCCCCC)CCCNC(=O)CC(C)C(=O)NCCCN(CCCCCCCC(=O)OC(CCCCCCCC)CCCCCCCC)CCCCCC(=O)OCCCCCCCCCCC] |
| | 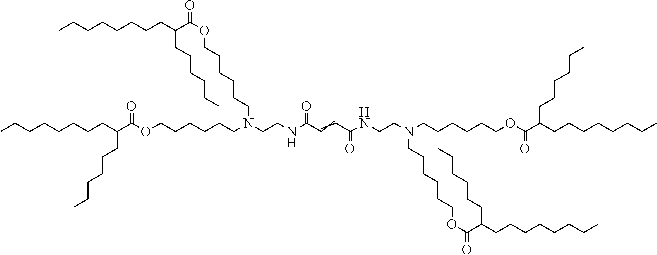 [SMILES: CCCCCCCCC(CCCCCC)C(=O)OCCCCCCN(CCCCCCOC(=O)C(CCCCCC)CCCCCCCC)CCNC(=O)C=CC(=O)NCCN(CCCCCCOC(=O)C(CCCCCC)CCCCCCCC)CCCCCCOC(=O)C(CCCCCC)CCCCCCCC] |
| | 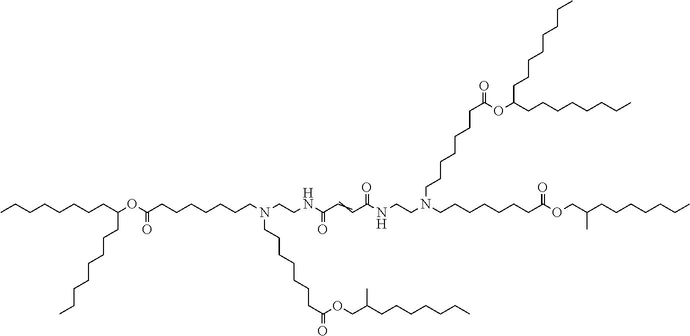 [SMILES: CCCCCCCCC(CCCCCCCC)OC(=O)CCCCCCCN(CCCCCCCC(=O)OCC(C)CCCCCCC)CCNC(=O)C=CC(=O)NCCN(CCCCCCCC(=O)OCC(C)CCCCCCC)CCCCCCCC(=O)OC(CCCCCCCC)CCCCCCCC] |
| | 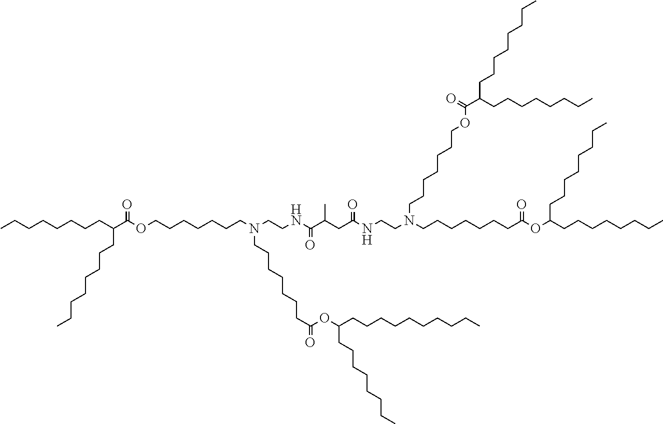 [SMILES: CCCCCCCCCCC(CCCCCCCC)OC(=O)CCCCCCCN(CCCCCCCOC(=O)C(CCCCCCCC)CCCCCCCC)CCNC(=O)C(C)CC(=O)NCCN(CCCCCCCOC(=O)C(CCCCCCCC)CCCCCCCC)CCCCCCCC(=O)OC(CCCCCCCC)CCCCCCCC] |
| | 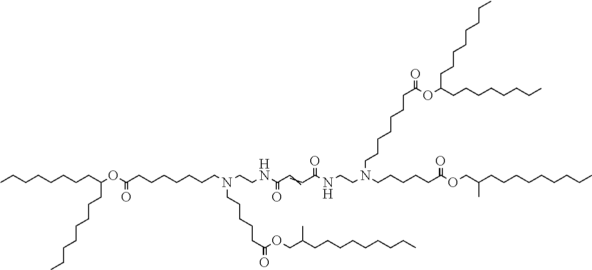 [SMILES: CCCCCCCCCC(C)COC(=O)CCCCCN(CCCCCCCC(=O)OC(CCCCCCCC)CCCCCCCC)CCNC(=O)C=CC(=O)NCCN(CCCCCCCC(=O)OC(CCCCCCCC)CCCCCCCC)CCCCCC(=O)OCC(C)CCCCCCCCC] |
| | 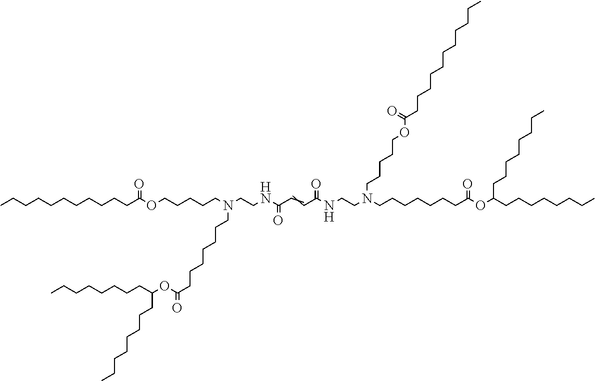 [SMILES: CCCCCCCCCCCC(=O)OCCCCCN(CCCCCCCC(=O)OC(CCCCCCCC)CCCCCCCC)CCNC(=O)C=CC(=O)NCCN(CCCCCCCC(=O)OC(CCCCCCCC)CCCCCCCC)CCCCCOC(=O)CCCCCCCCCCC] |
| | 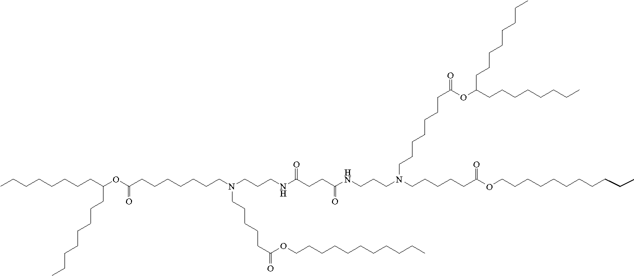 [SMILES: CCCCCCCCCCCOC(=O)CCCCCN(CCCCCCCC(=O)OC(CCCCCCCC)CCCCCCCC)CCCNC(=O)CCC(=O)NCCCN(CCCCCCCC(=O)OC(CCCCCCCC)CCCCCCCC)CCCCCC(=O)OCCCCCCCCCCC] |
| | 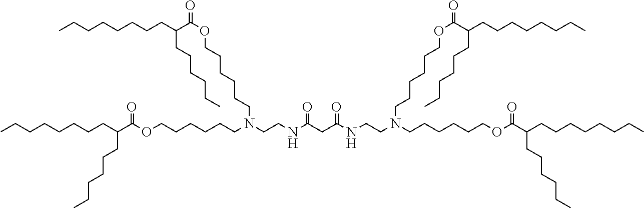 [SMILES: CCCCCCCCC(CCCCCC)C(=O)OCCCCCCN(CCCCCCOC(=O)C(CCCCCC)CCCCCCCC)CCNC(=O)CC(=O)NCCN(CCCCCCOC(=O)C(CCCCCC)CCCCCCCC)CCCCCCOC(=O)C(CCCCCC)CCCCCCCC] |
| | 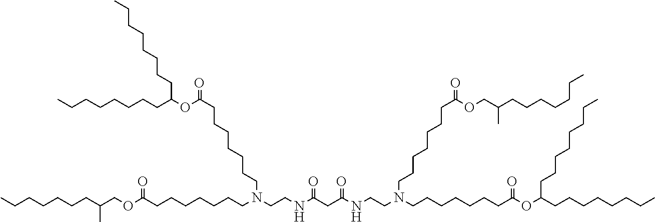 [SMILES: CCCCCCCCC(CCCCCCCC)OC(=O)CCCCCCCN(CCCCCCCC(=O)OCC(C)CCCCCCC)CCNC(=O)CC(=O)NCCN(CCCCCCCC(=O)OCC(C)CCCCCCC)CCCCCCCC(=O)OC(CCCCCCCC)CCCCCCCC] |
| | 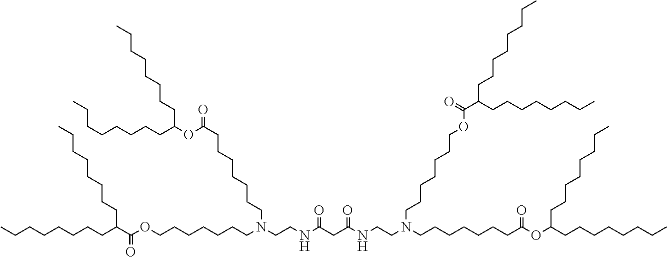 [SMILES: CCCCCCCCC(CCCCCCCC)OC(=O)CCCCCCCN(CCCCCCCOC(=O)C(CCCCCCCC)CCCCCCCC)CCNC(=O)CC(=O)NCCN(CCCCCCCOC(=O)C(CCCCCCCC)CCCCCCCC)CCCCCCCC(=O)OC(CCCCCCCC)CCCCCCCC] |
| | 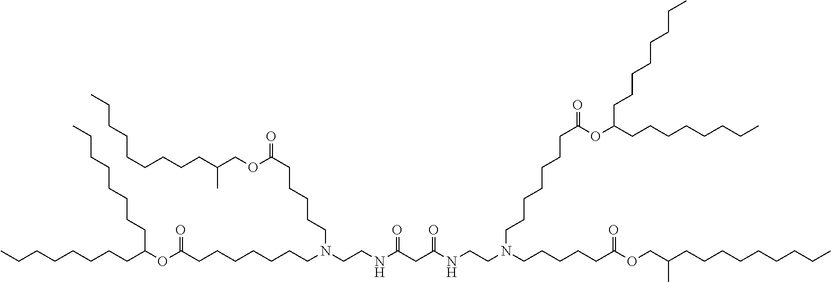 [SMILES: CCCCCCCCCC(C)COC(=O)CCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCC(=O)OCC(C)CCCCCCCCC] |
| | 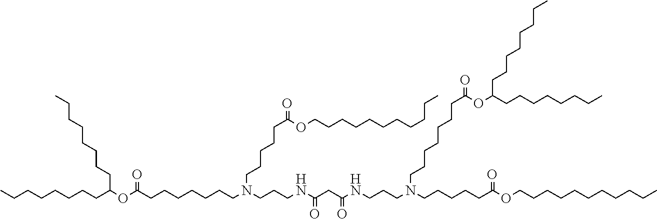 [SMILES: CCCCCCCCCCCOC(=O)CCCCCN(CCCCCCCC(=O)OC(CCCCCCCC)CCCCCCCC)CCCNC(=O)CC(=O)NCCCN(CCCCCCCC(=O)OC(CCCCCCCC)CCCCCCCC)CCCCCC(=O)OCCCCCCCCCCC] |
| | 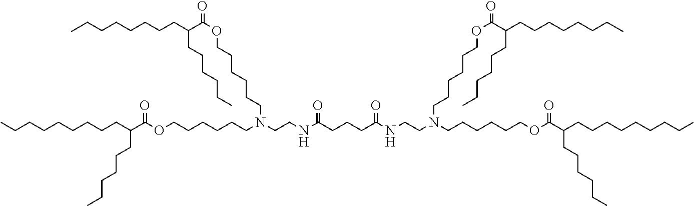 [SMILES: CCCCCCCCCC(CCCCCC)C(=O)OCCCCCCN(CCCCCCOC(=O)C(CCCCCC)CCCCCCCC)CCNC(=O)CCCC(=O)NCCN(CCCCCCOC(=O)C(CCCCCC)CCCCCCCC)CCCCCCOC(=O)C(CCCCCC)CCCCCCCCC] |
| | 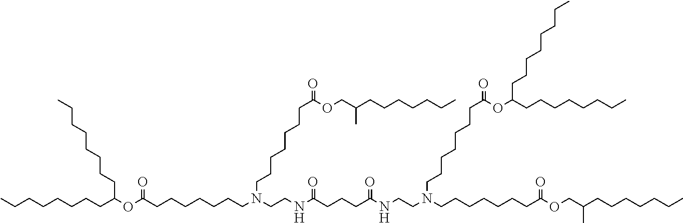 [SMILES: CCCCCCCCC(CCCCCCCC)OC(=O)CCCCCCCN(CCCCCCCC(=O)OCC(C)CCCCCCC)CCNC(=O)CCCC(=O)NCCN(CCCCCCCC(=O)OCC(C)CCCCCCC)CCCCCCCC(=O)OC(CCCCCCCC)CCCCCCCC] |
| | 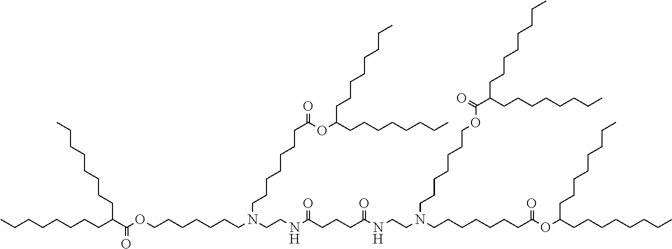 [SMILES: CCCCCCCCC(CCCCCCCC)OC(=O)CCCCCCCN(CCCCCCCOC(=O)C(CCCCCCCC)CCCCCCCC)CCNC(=O)CCCC(=O)NCCN(CCCCCCCOC(=O)C(CCCCCCCC)CCCCCCCC)CCCCCCCC(=O)OC(CCCCCCCC)CCCCCCCC] |
| | 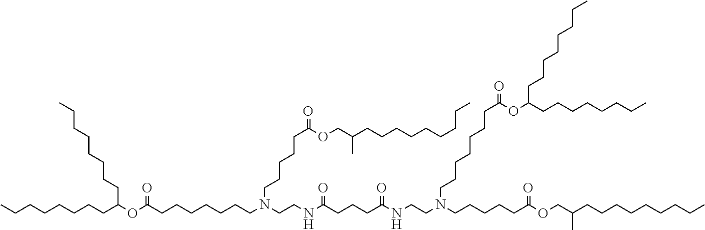 [SMILES: CCCCCCCCCC(C)COC(=O)CCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCC(=O)OCC(C)CCCCCCCCC] |
| | 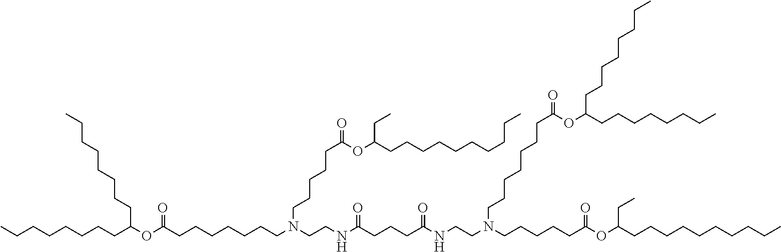 [SMILES: CCCCCCCCCCC(CC)OC(=O)CCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCC(=O)OC(CC)CCCCCCCCCC] |
| | 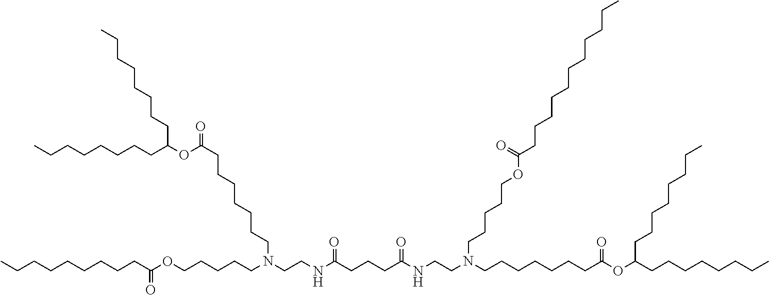 |
| | 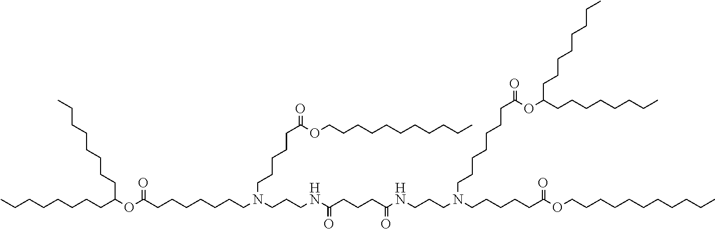 [SMILES: CCCCCCCCCCCOC(=O)CCCCCN(CCCCCCCC(=O)OC(CCCCCCCC)CCCCCCCC)CCCNC(=O)CCCC(=O)NCCCN(CCCCCCCC(=O)OC(CCCCCCCC)CCCCCCCC)CCCCCC(=O)OCCCCCCCCCCC] |
| | 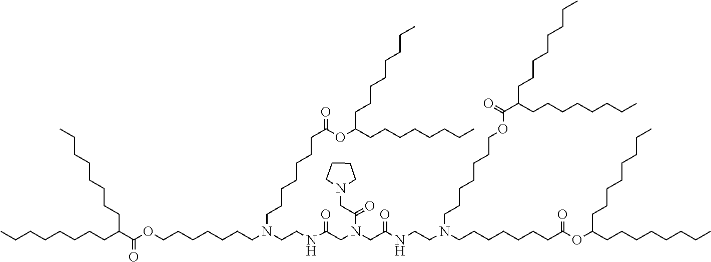 [SMILES: CCCCCCCCC(CCCCCCCC)OC(=O)CCCCCCCN(CCCCCCCOC(=O)C(CCCCCCCC)CCCCCCCC)CCNC(=O)CN(CC(=O)NCCN(CCCCCCCOC(=O)C(CCCCCCCC)CCCCCCCC)CCCCCCCC(=O)OC(CCCCCCCC)CCCCCCCC)C(=O)CN1CCCC1] |
| | 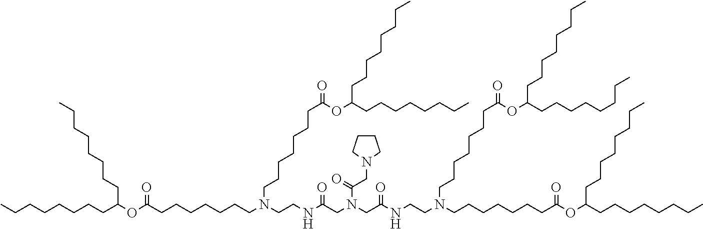 [SMILES: CCCCCCCCC(CCCCCCCC)OC(=O)CCCCCCCN(CCCCCCCC(=O)OC(CCCCCCCC)CCCCCCCC)CCNC(=O)CN(CC(=O)NCCN(CCCCCCCC(=O)OC(CCCCCCCC)CCCCCCCC)CCCCCCCC(=O)OC(CCCCCCCC)CCCCCCCC)C(=O)CN1CCCC1] |
| | 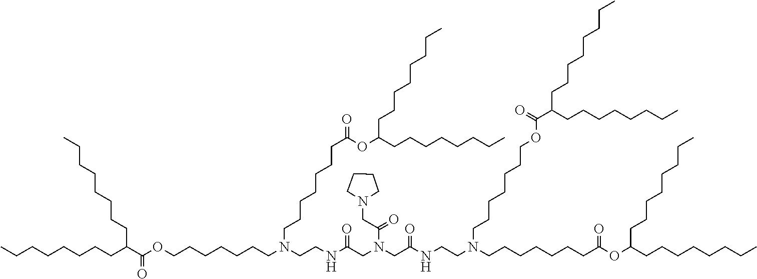 [SMILES: CCCCCCCCC(CCCCCCCC)OC(=O)CCCCCCCN(CCCCCCCOC(=O)C(CCCCCCCC)CCCCCCCC)CCNC(=O)CN(CC(=O)NCCN(CCCCCCCOC(=O)C(CCCCCCCC)CCCCCCCC)CCCCCCCC(=O)OC(CCCCCCCC)CCCCCCCC)C(=O)CN1CCCC1] |
| | 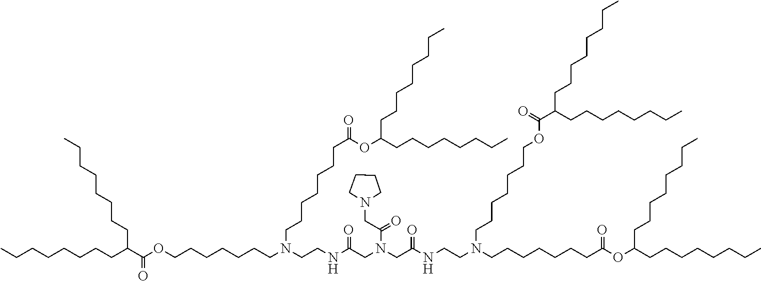 [SMILES: CCCCCCCCC(CCCCCCCC)OC(=O)CCCCCCCN(CCCCCCCOC(=O)C(CCCCCCCC)CCCCCCCC)CCNC(=O)CN(CC(=O)NCCN(CCCCCCCOC(=O)C(CCCCCCCC)CCCCCCCC)CCCCCCCC(=O)OC(CCCCCCCC)CCCCCCCC)C(=O)CN1CCCC1] |
| | 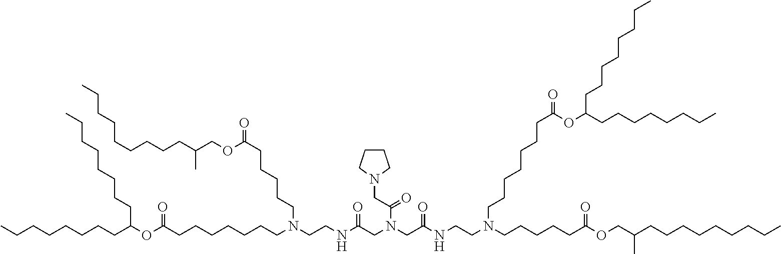 [SMILES: CCCCCCCCCC(C)COC(=O)CCCCCN(CCCCCCCC(=O)OC(CCCCCCCC)CCCCCCCC)CCNC(=O)CN(CC(=O)NCCN(CCCCCCCC(=O)OC(CCCCCCCC)CCCCCCCC)CCCCCC(=O)OCC(C)CCCCCCCCC)C(=O)CN1CCCC1] |
| | 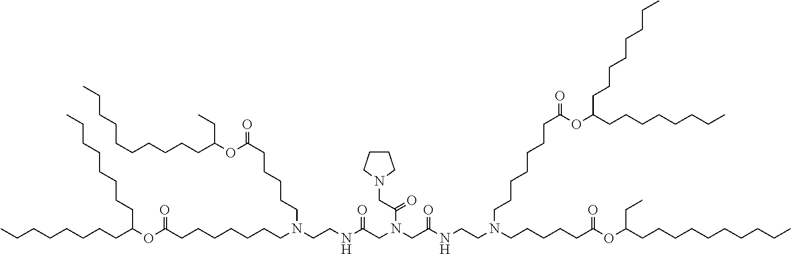 [SMILES: CCCCCCCCCCC(CC)OC(=O)CCCCCN(CCCCCCCC(=O)OC(CCCCCCCC)CCCCCCCC)CCNC(=O)CN(CC(=O)NCCN(CCCCCCCC(=O)OC(CCCCCCCC)CCCCCCCC)CCCCCC(=O)OC(CC)CCCCCCCCCC)C(=O)CN1CCCC1] |
| | 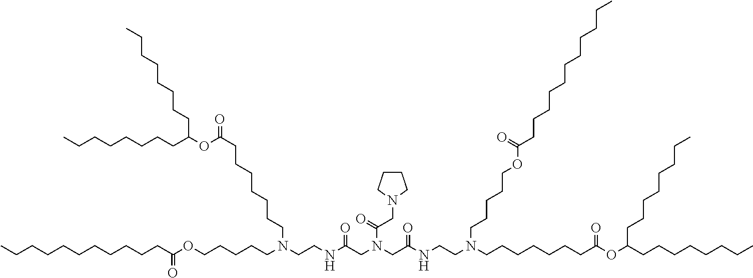 |
| | 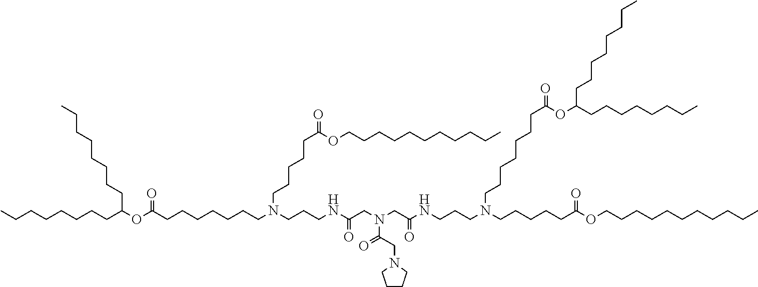 [SMILES: CCCCCCCCCCCOC(=O)CCCCCN(CCCCCCCC(=O)OC(CCCCCCCC)CCCCCCCC)CCCNC(=O)CN(CC(=O)NCCCN(CCCCCCCC(=O)OC(CCCCCCCC)CCCCCCCC)CCCCCC(=O)OCCCCCCCCCCC)C(=O)CN1CCCC1] |
| | 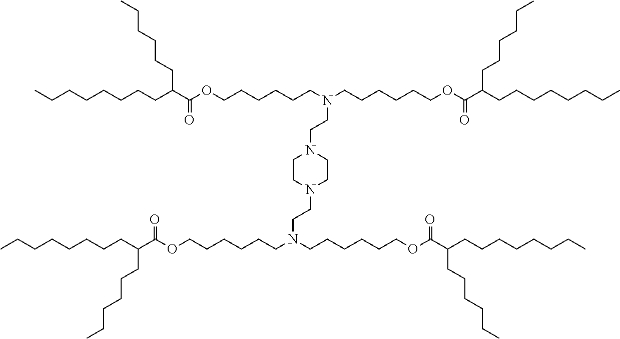 |
| | 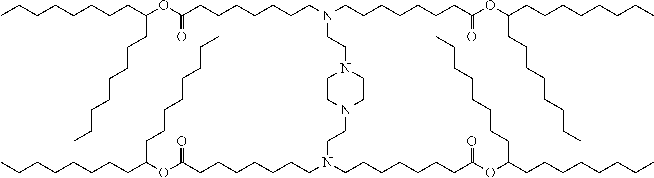 [SMILES: CCCCCCCCC(CCCCCCCC)OC(=O)CCCCCCCN(CCCCCCCC(=O)OC(CCCCCCCC)CCCCCCCC)CCN1CCN(CCN(CCCCCCCC(=O)OC(CCCCCCCC)CCCCCCCC)CCCCCCCC(=O)OC(CCCCCCCC)CCCCCCCC)CC1] |
| | 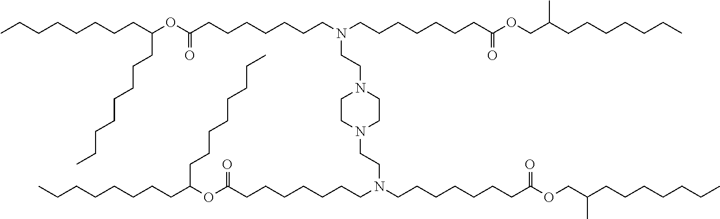 |
| | 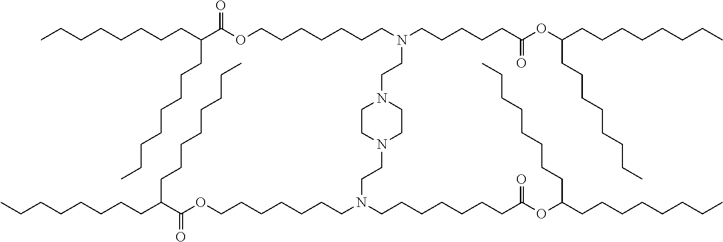 |
| | 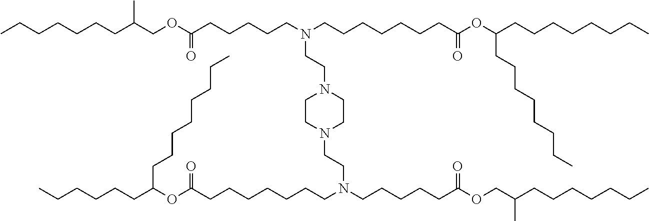 |
| | 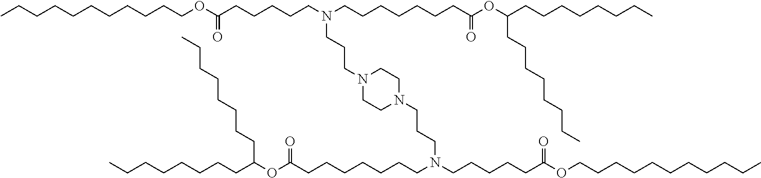 [SMILES: CCCCCCCCCCCOC(=O)CCCCCN(CCCCCCCC(=O)OC(CCCCCCCC)CCCCCCCC)CCCN1CCN(CCCN(CCCCCCCC(=O)OC(CCCCCCCC)CCCCCCCC)CCCCCC(=O)OCCCCCCCCCCC)CC1] |

Exemplary ionizable lipid compounds.

| Lipid | |
|---|---|
| No. | Name |
| 2303 | heptadecan-9-yl 8-[(3- |
| | {bis[8-(heptadecan-9- |
| | yloxy)-8-oxooctyl]amino}- |
| | 2-hydroxypropyl)[8- |
| | (heptadecan-9-yloxy)-8- |
| | oxooctyl]amino]octanoate |
| 2302 | undecyl 6-[(3-{bis[6-oxo- |
| | 6- |
| | (undecyloxy)hexyl]amino}- |
| | 2-hydroxypropyl)[6-oxo- |
| | 6- |
| | (undecyloxy)hexyl]amino] |
| | hexanoate |
| 2299 | heptadecan-9-yl 8-[(2-{4- |
| | [4-(2-{[8-(heptadecan-9- |
| | yloxy)-8-oxooctyl][6-oxo- |
| | 6- |
| | (undecyloxy)hexyl]amino} |
| | ethyl)piperazine-1- |
| | carbonyl]piperazin-1- |
| | yl}ethyl)[6-oxo-6- |
| | (undecyloxy)hexyl]amino] |
| | octanoate |
| 2289 | heptadecan-9-yl 8-({2-[2- |
| | ({[(2-{[8-(heptadecan-9- |
| | yloxy)-8-oxooctyl][6-oxo- |
| | 6- |
| | (undecyloxy)hexyl]amino} |
| | ethyl)carbamoyl]methyl}[3- |
| | (pyrrolidin-1- |
| | yl)propyl]amino)acetamido] |
| | ethyl}[6-oxo-6- |
| 2288 | heptadecan-9-yl 8-({2-[2- |
| | (N-{[(2-{[8-(heptadecan-9- |
| | yloxy)-8-oxooctyl][6-oxo- |
| | 6- |
| | (undecyloxy)hexyl]amino} |
| | ethyl)carbamoyl]methyl}- |
| | 3-(pyrrolidin-1- |
| | yl)propanamido)acetamido] |
| | ethyl}[6-oxo-6- |
| 2286 | heptadecan-9-yl 8-({2-[2- |
| | (dimethylamino)-3-[(2-{[8- |
| | (heptadecan-9-yloxy)-8- |
| | oxooctyl][6-oxo-6- |
| | (undecyloxy)hexyl]amino} |
| | ethyl)carbamoyl] |
| | propanamido]ethyl}[6-oxo-6- |
| | (undecyloxy)hexyl]amino) |
| | octanoate |
| 2285 | heptadecan-9-yl 8-[(2-{3- |
| | [(2-{[8-(heptadecan-9- |
| | yloxy)-8-oxooctyl][6-oxo- |
| | 6- |
| | (undecyloxy)hexyl]amino} |
| | ethyl)carbamoyl]-2- |
| | hydroxypropanamido} |
| | ethyl)[6-oxo-6- |
| | (undecyloxy)hexyl]amino] |
| | octanoate |
| 2284 | heptadecan-9-yl 8-[(2-{3- |
| | [(2-{[8-(heptadecan-9- |
| | yloxy)-8-oxooctyl][6-oxo- |
| | 6- |
| | (undecyloxy)hexyl]amino} |
| | ethyl)carbamoyl]-2- |
| | methylpropanamido}ethyl) |
| | [6-oxo-6- |
| | (undecyloxy)hexyl]amino] |
| | octanoate |
| 2283 | N,N′-bis[2-({7- |
| | [(heptadecan-9- |
| | yl)(methyl)carbamoyl] |
| | heptyl}({5- |
| | [methyl(undecyl)carbamoyl] |
| | pentyl})amino)ethyl] |
| | butanediamide |
| 2282 | N, N′-bis [2-({7- |
| | [(heptadecan-9- |
| | yl)carbamoyl]heptyl}[5- |
| | (undecylcarbamoyl)pentyl] |
| | amino)ethyl]butanediamide |
| 2281 | heptadecan-9-yl 8-[(2-{3- |
| | [(2-{[8-(heptadecan-9- |
| | yloxy)-8-oxooctyl][6-oxo- |
| | 6- |
| | (undecyloxy)hexyl]amino} |
| | ethyl)carbamoyl]-N- |
| | methylpropanamido} |
| | ethyl)[6-oxo-6- |
| | (undecyloxy)hexyl]amino] |
| | octanoate |
| 2280 | heptadecan-9-yl 8-[(2-{3- |
| | [(2-{[8-(heptadecan-9- |
| | yloxy)-8-oxooctyl][6-oxo- |
| | 6- |
| | (undecyloxy)hexyl]amino} |
| | ethyl)(methyl)carbamoyl]- |
| | N- |
| | methylpropanamido}ethyl) |
| | [6-oxo-6- |
| | (undecyloxy)hexyl]amino] |
| | octanoate |
| 2279 | 6-({2-[3-({2-[bis({6-[(2- |
| | hexyldecanoyl)oxy]hexyl}) |
| | amino]ethyl}carbamoyl) |
| | propanamido]ethyl}({6-[(2- |
| | hexyldecanoyl)oxy]hexyl}) |
| | amino)hexyl 2- |
| | hexyldecanoate |
| 2278 | 5-[(2-{3-[(2-{[5- |
| | (dodecanoyloxy)pentyl]({7- |
| | [(2- |
| | octyldecanoyl)oxy]heptyl}) |
| | amino}ethyl)carbamoyl] |
| | propanamido}ethyl)({7-[(2- |
| | octyldecanoyl)oxy]heptyl}) |
| | amino]pentyl |
| | dodecanoate |
| 2275 | heptadecan-9-yl 8-[(2-{3- |
| | [(2-{bis[8-(heptadecan-9- |
| | yloxy)-8- |
| | oxooctyl]amino}ethyl) |
| | carbamoyl]propanamido}ethyl) |
| | [8-(heptadecan-9-yloxy)-8- |
| | oxooctyl]amino]octanoate |
| 2274 | heptadecan-9-yl 8-[(3-{3- |
| | [(3-{[8-(heptadecan-9- |
| | yloxy)-8-oxooctyl][6-oxo- |
| | 6- |
| | (undecyloxy)hexyl]amino} |
| | propyl)carbamoyl] |
| | propanamido}propyl)[6-oxo- |
| | 6-(undecyloxy)hexyl]amino] |
| | octanoate |
| 2273 | heptadecan-9-yl 8-[(2-{4- |
| | [(2-{[8-(heptadecan-9- |
| | yloxy)-8-oxooctyl][6-oxo- |
| | 6- |
| | (undecyloxy)hexyl]amino} |
| | ethyl)carbamoyl] |
| | butanamido}ethyl)[6-oxo-6- |
| | (undecyloxy)hexyl]amino] |
| | octanoate |
| 2272 | 7-[(2-{3-[(2-{[7- |
| | (decanoyloxy)heptyl][8- |
| | (heptadecan-9-yloxy)-8- |
| | oxooctyl]amino}ethyl) |
| | carbamoyl]propanamido}ethyl) |
| | [8-(heptadecan-9-yloxy)- |
| | 8-oxooctyl]amino]heptyl |
| | decanoate |
| 2271 | heptadecan-9-yl 8-[(2-{3- |
| | [(2-{[8-(heptadecan-9- |
| | yloxy)-8-oxooctyl] ({8-[(2- |
| | methylnonyl)oxy]-8- |
| | oxooctyl})amino}ethyl) |
| | carbamoyl]propanamido} |
| | ethyl)({8-[(2- |
| | methylnonyl)oxy]-8- |
| | oxooctyl})amino]octanoate |
| 2253 | heptadecan-9-yl 8-{[2- |
| | ({[8-(heptadecan-9- |
| | yloxy)-8-oxooctyl][6-oxo- |
| | 6- |
| | (undecyloxy)hexyl]amino} |
| | methyl)prop-2-en-1-yl][6- |
| | oxo-6- |
| | (undecyloxy)hexyl]amino} |
| | octanoate |
| 2252 | heptadecan-9-yl 8-({2- |
| | [(2E)-3-[(2-{[8- |
| | (heptadecan-9-yloxy)-8- |
| | oxooctyl][6-oxo-6- |
| | (undecyloxy)hexyl]amino} |
| | ethyl)carbamoyl]prop-2- |
| | enamido]ethyl}[6-oxo-6- |
| | (undecyloxy)hexyl]amino) |
| | octanoate |
| 2251 | heptadecan-9-yl 8-{[2- |
| | ({[8-(heptadecan-9- |
| | yloxy)-8-oxooctyl][6-oxo- |
| | 6- |
| | (undecyloxy)hexyl]amino} |
| | methyl)-3- |
| | hydroxypropyl][6-oxo-6- |
| | (undecyloxy)hexyl]amino} |
| | octanoate |
| 2247 | heptadecan-9-yl 8-[(2- |
| | {[(2-{[8-(heptadecan-9- |
| | yloxy)-8-oxooctyl][6-oxo- |
| | 6- |
| | (undecyloxy)hexyl]amino} |
| | ethoxy)carbonyl]amino} |
| | ethyl)[6-oxo-6- |
| | (undecyloxy)hexyl]amino] |
| | octanoate |
| 2246 | heptadecan-9-yl 8-[(2- |
| | {[(2-{[8-(heptadecan-9- |
| | yloxy)-8-oxooctyl][6-oxo- |
| | 6- |
| | (undecyloxy)hexyl]amino} |
| | ethyl)carbamoyl]amino} |
| | ethyl)[6-oxo-6- |
| | (undecyloxy)hexyl]amino] |
| | octanoate |
| 2221 | heptadecan-9-yl 8-[(2-{3- |
| | [(2-{[8-(heptadecan-9- |
| | yloxy)-8-oxooctyl][6-oxo- |
| | 6- |
| | (undecyloxy)hexyl]amino} |
| | ethyl)carbamoyl]propanamido} |
| | ethyl)[6-oxo-6- |
| | (undecyloxy)hexyl]amino] |
| | octanoate |
| 2220 | heptadecan-9-yl 8-[(2-{2- |
| | [(2-{[8-(heptadecan-9- |
| | yloxy)-8-oxooctyl][6-oxo- |
| | 6- |
| | (undecyloxy)hexyl]amino} |
| | ethyl)carbamoyl]acetamido} |
| | ethyl)[6-oxo-6- |
| | (undecyloxy)hexyl]amino] |
| | octanoate |
| 2219 | heptadecan-9-yl 8-[(2-{3- |
| | [(2-{[8-(heptadecan-9- |
| | yloxy)-8-oxooctyl][6-oxo- |
| | 6- |
| | (undecyloxy)hexyl]amino} |
| | ethyl)carbamoyl]-2,3- |
| | dihydroxypropanamido} |
| | ethyl)[6-oxo-6- |
| | (undecyloxy)hexyl]amino] |
| | octanoate |
| 2218 | heptadecan-9-yl 8-[(2- |
| | {[(2-{[8-(heptadecan-9- |
| | yloxy)-8-oxooctyl][6-oxo- |
| | 6- |
| | (undecyloxy)hexyl]amino} |
| | ethoxy)carbonyl]oxy}ethyl) |
| | [6-oxo-6- |
| | (undecyloxy)hexyl]amino] |
| | octanoate |
| 2217 | heptadecan-9-yl 8-[(3-{[8- |
| | (heptadecan-9-yloxy)-8- |
| | oxooctyl][6-oxo-6- |
| | (undecyloxy)hexyl]amino}- |
| | 2-hydroxypropyl)[6-oxo- |
| | 6- |
| | (undecyloxy)hexyl]amino] |
| | octanoate |
| 2213 | heptadecan-9-yl 8-[(2-{3- |
| | [(2-{[8-(heptadecan-9- |
| | yloxy)-8-oxooctyl][8- |
| | (nonyloxy)-8- |
| | oxooctyl]amino}ethyl) |
| | carbamoyl]propanamido} |
| | ethyl)[8-(nonyloxy)-8- |
| | oxooctyl]amino]octanoate |
| 2212 | heptadecan-9-yl 8-[(2-{2- |
| | [(2-{[8-(heptadecan-9- |
| | yloxy)-8-oxooctyl][8- |
| | (nonyloxy)-8- |
| | oxooctyl]amino}ethyl)carb |
| | amoyl]acetamido}ethyl)[8- |
| | (nonyloxy)-8- |
| | oxooctyl]amino]octanoate |
| 2211 | heptadecan-9-yl 8-[(2-{3- |
| | [(2-{[8-(heptadecan-9- |
| | yloxy)-8-oxooctyl][8- |
| | (nonyloxy)-8- |
| | oxooctyl]amino}ethyl) |
| | carbamoyl]-2,3- |
| | dihydroxypropanamido} |
| | ethyl)[8-(nonyloxy)-8- |
| | oxooctyl]amino]octanoate |
| 2210 | heptadecan-9-yl 8-[(2- |
| | {[(2-{[8-(heptadecan-9- |
| | yloxy)-8-oxooctyl][8- |
| | (nonyloxy)-8- |
| | oxooctyl]amino}ethoxy)ca |
| | rbonyl]oxy}ethyl)[8- |
| | (nonyloxy)-8- |
| | oxooctyl]amino]octanoate |
| 2209 | heptadecan-9-yl 8-[(3-{[8- |
| | (heptadecan-9-yloxy)-8- |
| | oxooctyl][8-(nonyloxy)-8- |
| | oxooctyl]amino}-2- |
| | hydroxypropyl)[8- |
| | (nonyloxy)-8- |
| | oxooctyl]amino]octanoate |
| 2513 | di(heptadecan-9-yl) |
| | 14,17-dioxo-9,22-bis(6- |
| | oxo-6-(tridecan-3- |
| | yloxy)hexyl)-9,13,18,22- |
| | tetraazatriacontanedioate |
| | tetrakis(4-pentylnonyl) |
| | 8,8′,8″,8′′′-((2-(pyrrolidin- |
| | 1- |
| | ylmethyl)malonyl)bis |
| | (azanetriyl)) tetraoctanoate |
| | di(heptadecan-9-yl) 16- |
| | methyl-13,20-dioxo-9,24- |
| | bis(6-oxo-6- |
| | (undecyloxy)hexyl)- |
| | 9,12,16,21,24- |
| | pentaazadotriacontanedioate |
| | di(heptadecan-9-yl) |
| | 13,20-dioxo-9,24-bis(6- |
| | oxo-6-(undecyloxy)hexyl)- |
| | 16-oxa-9,12,21,24- |
| | tetraazadotriacontanedioate |
| 2248 | heptadecan-9-yl 8-({2-[4- |
| | (2-{[8-(heptadecan-9- |
| | yloxy)-8-oxooctyl][6-oxo- |
| | 6- |
| | (undecyloxy)hexyl]amino} |
| | ethyl)piperazin-1- |
| | yl]ethyl}[6-oxo-6- |
| | (undecyloxy)hexyl]amino) |
| | octanoate |
| 2190 | 5-hexylundecyl 8-({4- |
| | [(2S,5S)-5-{4-[bis({8-[(5- |
| | hexylundecyl)oxy]-2- |
| | hydroxy-8- |
| | oxooctyl})amino]butyl}- |
| | 3,6-dioxopiperazin-2- |
| | yl]butyl} {{8-[(5- |
| | hexylundecyl)oxy]-2- |
| | hydroxy-8- |
| | oxooctyl})amino)-7- |
| | hydroxyoctanoate |
| 2189 | heptadecan-9-yl 8-({4- |
| | [(2S,5S)-5-(4-{bis[8- |
| | (heptadecan-9-yloxy)-2- |
| | hydroxy-8- |
| | oxooctyl]amino}butyl)-3,6- |
| | dioxopiperazin-2- |
| | yl]butyl}[8-(heptadecan-9- |
| | yloxy)-2-hydroxy-8- |
| | oxooctyl]amino)-7- |
| | hydroxyoctanoate |
| 2287 | di(heptadecan-9-yl) 14- |
| | (2-(dimethylamino)ethyl)- |
| | 13,16-dioxo-9,20-bis(6- |
| | oxo-6-(undecyloxy)hexyl)- |
| | 9,12,17,20- |
| | tetraazaoctacosanedioate |
| 2315 | di(heptadecan-9-yl) 8,8′- |
| | (((2- |
| | (hydroxymethyl)piperazine- |
| | 1,4-diyl)bis(ethane-2,1- |
| | diyl)) bis((6-oxo-6- |
| | (undecyloxy)hexyl) |
| | azanediyl)) (R)-dioctanoate |
| 2316 | heptadecan-9-yl (S)-8- |
| | ((2-(4-(1-((8-(heptadecan- |
| | 9-yloxy)-8-oxooctyl)(6- |
| | oxo-6- |
| | (undecyloxy)hexyl)amino)- |
| | 3-hydroxypropan-2- |
| | yl)piperazin-1-yl)ethyl)(6- |
| | 0x0-6- |
| | (undecyloxy)hexyl)amino) |
| | octanoate |
| 2317 | heptadecan-9-yl (S)-8- |
| | ((2-(4-(2-((8-(heptadecan- |
| | 9-yloxy)-8-oxooctyl)(6- |
| | ox0-6- |
| | (undecyloxy)hexyl) |
| | amino)-3- |
| | hydroxypropyl)piperazin- |
| | 1-yl)ethyl)(6-oxo-6- |
| | (undecyloxy)hexyl)amino) |
| | octanoate |
| 2318 | di(heptadecan-9-yl) |
| | 13,15-dioxo-9, 19-bis(5- |
| | (undecyldisulfaneyl)pentyl)- |
| | 9,12,16,19- |
| | tetraazaheptacosanedioate |
| 2439 | di(heptadecan-9-yl) |
| | 13,16-dioxo-9,20-bis(5- |
| | (undecyldisulfaneyl)pentyl)- |
| | 9,12,17,20- |
| | tetraazaoctacosanedioate |
| 2319 | di(heptadecan-9-yl) 9,19- |
| | bis(8-(heptadecan-9- |
| | yloxy)-8-oxooctyl)-13,15- |
| | dioxo-9,12,16,19- |
| | tetraazaheptacosanedioate |
| 2320 | di(heptadecan-9-yl) 9,21- |
| | bis(8-(heptadecan-9- |
| | yloxy)-8-oxooctyl)-13,17- |
| | dioxo-9,12,18,21- |
| | tetraazanonacosanedioate |
| 2321 | di(heptadecan-9-yl) (E)- |
| | 9,20-bis(8-(heptadecan- |
| | 9-yloxy)-8-oxooctyl)- |
| | 13,16-dioxo-9,12,17,20- |
| | tetraazaoctacos-14- |
| | enedioate |
| 2322 | di(heptadecan-9-yl) 9,17- |
| | bis(8-(heptadecan-9- |
| | yloxy)-8-oxooctyl)-13- |
| | oxo-9,12,14,17- |
| | tetraazapentacosanedioate |
| 2323 | di(heptadecan-9-yl) 9,20- |
| | bis(7-((2- |
| | octyldecanoyl)oxy)heptyl) |
| | -13,16-dioxo-9,12,17,20- |
| | tetraazaoctacosanedioate |
| 2324 | di(heptadecan-9-yl) |
| | 10,18-dimethyl-13,15- |
| | dioxo-9, 19-bis(6-oxo-6- |
| | (undecyloxy)hexyl)- |
| | 9,12,16,19- |
| | tetraazaheptacosanedioate |
| 2325 | di(heptadecan-9-yl) |
| | 10,19-dimethyl-13,16- |
| | dioxo-9,20-bis(6-oxo-6- |
| | (undecyloxy)hexyl)- |
| | 9,12,17,20- |
| | tetraazaoctacosanedioate |
| 2326 | di(heptadecan-9-yl) (E)- |
| | 10,19-dimethyl-13,16- |
| | dioxo-9,20-bis(6-oxo-6- |
| | (undecyloxy)hexyl)- |
| | 9,12,17,20- |
| | tetraazaoctacos-14- |
| | enedioate |
| 2327 | di(heptadecan-9-yl) |
| | 10,19-dimethyl-9,20- |
| | bis(8-(nonyloxy)-8- |
| | oxooctyl)-13,16-dioxo- |
| | 9,12,17,20- |
| | tetraazaoctacosanedioate |
| 2328 | di(heptadecan-9-yl) 9,20- |
| | bis(8-(heptadecan-9- |
| | yloxy)-8-oxooctyl)-10,19- |
| | dimethyl-13,16-dioxo- |
| | 9,12,17,20- |
| | tetraazaoctacosanedioate |
| 2332 | bis(4-pentylnonyl) 13,16- |
| | dioxo-9,20-bis(8-oxo-8- |
| | ((4- |
| | pentylnonyl)oxy)octyl)- |
| | 9,12,17,20- |
| | tetraazaoctacosanedioate |
| 2334 | di(heptadecan-9-yl) |
| | 13,17-dioxo-9,21-bis(6- |
| | ox0-6-(undecyloxy)hexyl)- |
| | 15-(2-(pyrrolidin-1- |
| | yl)acetyl)-9,12,15,18,21- |
| | pentaazanonacosanedioate |
| 2353 | di(heptadecan-9-yl) |
| | 14,18-dioxo-9,23-bis(6- |
| | oxo-6-(undecyloxy)hexyl)- |
| | 16-(3-(pyrrolidin-1- |
| | yl)propanoyl)- |
| | 9,13,16,19,23- |
| | pentaazahentriacontanedioate |
| 2354 | di(heptadecan-9-yl) 9,20- |
| | bis(5- |
| | (dodecanoyloxy)pentyl)- |
| | 13,16-dioxo-9,12,17,20- |
| | tetraazaoctacosanedioate |
| 2355 | di(heptadecan-9-yl) 9,20- |
| | bis (6-((2- |
| | methylundecyl)oxy)-6- |
| | oxohexyl)-13, 16-dioxo- |
| | 9,12,17,20- |
| | tetraazaoctacosanedioate |
| 2356 | di(heptadecan-9-yl) |
| | 13,16-dioxo-9,20-bis(6- |
| | oxo-6-(tridecan-3- |
| | yloxy)hexyl)-9,12,17,20- |
| | tetraazaoctacosanedioate |
| 2357 | di(heptadecan-9-yl) 15- |
| | (3-((2-(methylamino)-3,4- |
| | dioxocyclobut-1-en-1- |
| | yl)amino)propyl)-13,17- |
| | dioxo-9,21-bis(6-oxo-6- |
| | (undecyloxy)hexyl)- |
| | 9,12,15,18,21- |
| | pentaazanonacosanedioate |
| 2358 | di(heptadecan-9-yl) 15- |
| | (3-(3,3- |
| | dimethylthioureido)propyl)- |
| | 13,17-dioxo-9,21-bis(6- |
| | oxo-6-(undecyloxy)hexyl)- |
| | 9,12,15,18,21- |
| | pentaazanonacosanedioate |
| 2361 | di(heptadecan-9-yl) 15- |
| | (3-((2-(methylamino)-3,4- |
| | dioxocyclobut-1-en-1- |
| | yl)amino)propyl)-13,17- |
| | dioxo-9,21-bis(6-oxo-6- |
| | (tridecan-3-yloxy)hexyl)- |
| | 9,12,15,18,21- |
| | pentaazanonacosanedioate |
| 2362 | di(heptadecan-9-yl) 15- |
| | (3-(3,3- |
| | dimethylthioureido)propyl)- |
| | 13,17-dioxo-9,21-bis(6- |
| | oxo-6-(tridecan-3- |
| | yloxy)hexyl)- |
| | 9,12,15,18,21- |
| | pentaazanonacosanedioate |
| 2378 | di(heptadecan-9-yl) (E)- |
| | 13,16-dioxo-9,20-bis(6- |
| | oxo-6-(tridecan-3- |
| | yloxy)hexyl)-9,12,17,20- |
| | tetraazaoctacos-14- |
| | enedioate |
| 2379 | di(heptadecan-9-yl) (E)- |
| | 9,20-bis (6-((2- |
| | methylnonyl)oxy)-6- |
| | oxohexyl)-13,16-dioxo- |
| | 9,12,17,20- |
| | tetraazaoctacos-14- |
| | enedioate |
| 2380 | di(heptadecan-9-yl) (E)- |
| | 9,20-bis(7- |
| | (decanoyloxy)heptyl)- |
| | 13,16-dioxo-9,12,17,20- |
| | tetraazaoctacos-14- |
| | enedioate |
| 2381 | di(pentadecan-7-yl) (E)- |
| | 13,16-dioxo-9,20-bis(6- |
| | oxo-6-(undecyloxy)hexyl)- |
| | 9,12,17,20- |
| | tetraazaoctacos-14- |
| | enedioate |
| 2382 | bis(4-pentylnonyl) (E)- |
| | 13,16-dioxo-9,20-bis(6- |
| | oxo-6-(undecyloxy)hexyl)- |
| | 9,12,17,20- |
| | tetraazaoctacos-14- |
| | enedioate |
| 2384 | di(heptadecan-9-yl) |
| | 13,15-dioxo-9,19-bis(6- |
| | oxo-6-(tridecan-3- |
| | yloxy)hexyl)-9,12,16,19- |
| | tetraazaheptacosanedioate |
| 2385 | di(heptadecan-9-yl) 9,19- |
| | bis (6-((2- |
| | methylnonyl)oxy)-6- |
| | oxohexyl)-13, 15-dioxo- |
| | 9,12,16,19- |
| | tetraazaheptacosanedioate |
| 2386 | di(heptadecan-9-yl) 9,19- |
| | bis(7- |
| | (decanoyloxy)heptyl)- |
| | 13,15-dioxo-9,12,16,19- |
| | tetraazaheptacosanedioate |
| 2387 | di(pentadecan-7-yl) |
| | 13,15-dioxo-9,19-bis(6- |
| | oxo-6-(undecyloxy)hexyl)- |
| | 9,12,16,19- |
| | tetraazaheptacosanedioate |
| 2388 | bis(4-pentylnonyl) 13,15- |
| | dioxo-9,19-bis(6-oxo-6- |
| | (undecyloxy)hexyl)- |
| | 9,12,16,19- |
| | tetraazaheptacosanedioate |
| 2389 | di(heptadecan-9-yl) 9,20- |
| | bis(6-((2- |
| | methylnonyl)oxy)-6- |
| | oxohexyl)-13,16-dioxo- |
| | 9,12,17,20- |
| | tetraazaoctacosanedioate |
| 2390 | di(pentadecan-7-yl) |
| | 13,16-dioxo-9,20-bis(6- |
| | oxo-6-(undecyloxy)hexyl)- |
| | 9,12,17,20- |
| | tetraazaoctacosanedioate |
| 2391 | bis(4-pentylnonyl) 13,16- |
| | dioxo-9,20-bis(6-oxo-6- |
| | (undecyloxy)hexyl)- |
| | 9,12,17,20- |
| | tetraazaoctacosanedioate |
| 2393 | di(heptadecan-9-yl) 8,8′- |
| | ((piperazine-1,4- |
| | diylbis(ethane-2,1- |
| | diyl))bis((6-oxo-6- |
| | (tridecan-3- |
| | yloxy)hexyl)azanediyl)) |
| | dioctanoate |
| 2394 | di(heptadecan-9-yl) 8,8′- |
| | ((piperazine-1,4- |
| | diylbis(ethane-2,1- |
| | diyl)) bis ((6-((2- |
| | methylnonyl)oxy)-6- |
| | oxohexyl)azanediyl)) |
| | dioctanoate |
| 2395 | ((piperazine-1,4- |
| | diylbis(ethane-2,1- |
| | diyl)) bis((8-(heptadecan- |
| | 9-yloxy)-8- |
| | oxooctyl)azanediyl))bis |
| | (heptane-7,1-diyl) |
| | bis(decanoate) |
| 2396 | di(pentadecan-7-yl) 8,8′- |
| | ((piperazine-1,4- |
| | diylbis(ethane-2,1- |
| | diyl))bis((6-oxo-6- |
| | (undecyloxy)hexyl)azane |
| | diyl))dioctanoate |
| 2397 | bis(4-pentylnonyl) 8,8′- |
| | ((piperazine-1,4- |
| | diylbis(ethane-2,1- |
| | diyl)) bis((6-oxo-6- |
| | (undecyloxy)hexyl)azane |
| | diyl))dioctanoate |
| 2399 | ((piperazine-1,4- |
| | diylbis(ethane-2,1- |
| | diyl)) bis((8-(heptadecan- |
| | 9-yloxy)-8- |
| | oxooctyl)azanediyl))bis |
| | (pentane-5,1-diyl) |
| | didodecanoate |
| 2400 | di(heptadecan-9-yl) 8,8′- |
| | ((piperazine-1,4- |
| | diylbis(ethane-2,1- |
| | diyl)) bis((8-(nonyloxy)-8- |
| | oxooctyl)azanediyl)) |
| | dioctanoate |
| 2401 | ((piperazine-1,4- |
| | diylbis(ethane-2,1- |
| | diyl)) bis((7-((2- |
| | octyldecanoyl)oxy)heptyl) |
| | azanediyl)) bis (pentane- |
| | 5,1-diyl) didodecanoate |
| 2403 | 8,19-bis(7- |
| | (decanoyloxy)heptyl)- |
| | 12,15-dioxo-8,11,16,19- |
| | tetraazahexacosane- |
| | 1,26-diyl bis(2- |
| | octyldecanoate) |
| 2404 | 7-[2-[4-[2-[bis[7-(2- |
| | octyldecanoyloxy)heptyl] |
| | amino]ethylamino]-4-oxo- |
| | butanoyl]amino]ethyl-[7- |
| | (2- |
| | octyldecanoyloxy)heptyl] |
| | amino]heptyl 2- |
| | octyldecanoate |
| 2405 | diundecyl 7,18-bis(7-((2- |
| | octyldecanoyl)oxy)heptyl)- |
| | 11,14-dioxo-7,10,15,18- |
| | tetraazatetracosanedioate |
| 2421 | N1, N4-bis(2-((8- |
| | (heptadecan-9-ylamino)- |
| | 8-oxooctyl)(6-oxo-6- |
| | (tridecan-3- |
| | ylamino)hexyl)amino) |
| | ethyl)succinamide |
| 2426 | N1, N4-bis(2-(bis(8- |
| | (heptadecan-9-ylamino)- |
| | 8- |
| | oxooctyl)amino)ethyl) |
| | succinamide |
| 2427 | N1, N4-bis(2-((7- |
| | decanamidoheptyl)(8- |
| | (heptadecan-9-ylamino)- |
| | 8- |
| | oxooctyl)amino)ethyl) |
| | succinamide |
| 2428 | 8,8′-((piperazine-1,4- |
| | diylbis(ethane-2,1- |
| | diyl)) bis((6-oxo-6- |
| | (undecylamino)hexyl) |
| | azanediyl)) bis(N- |
| | (heptadecan-9- |
| | yl)octanamide) |
| | 6-[2-[4-[2-[bis[6-(2- |
| | hexyldecanoyloxy)hexyl] |
| | amino]ethylamino]-3- |
| | methyl-4-oxo- |
| | butanoyl]amino]ethyl-[6- |
| | (2- |
| | hexyldecanoyloxy)hexyl] |
| | amino]hexyl 2- |
| | hexyldecanoate |
| | di(heptadecan-9-yl) 9,20- |
| | bis(8-(heptadecan-9- |
| | yloxy)-8-oxooctyl)-14- |
| | methyl-13,16-dioxo- |
| | 9,12,17,20- |
| | tetraazaoctacosanedioate |
| | di(heptadecan-9-yl) 14- |
| | methyl-9,20-bis(8-((2- |
| | methylnonyl)oxy)-8- |
| | oxooctyl)-13, 16-dioxo- |
| | 9,12,17,20- |
| | tetraazaoctacosanedioate |
| | di(heptadecan-9-yl) 14- |
| | methyl-9,20-bis(7-((2- |
| | octyldecanoyl)oxy)heptyl)- |
| | 13,16-dioxo-9,12,17,20- |
| | tetraazaoctacosanedioate |
| | di(heptadecan-9-yl) 14- |
| | methyl-9,20-bis(6-((2- |
| | methylundecyl)oxy)-6- |
| | oxohexyl)-13, 16-dioxo- |
| | 9,12,17,20- |
| | tetraazaoctacosanedioate |
| | di(heptadecan-9-yl) 14- |
| | methyl-13, 16-dioxo-9,20- |
| | bis(6-oxo-6-(tridecan-3- |
| | yloxy)hexyl)-9,12,17,20- |
| | tetraazaoctacosanedioate |
| | di(heptadecan-9-yl) 9,20- |
| | bis(5- |
| | (dodecanoyloxy)pentyl)- |
| | 14-methyl-13,16-dioxo- |
| | 9,12,17,20- |
| | tetraazaoctacosanedioate |
| | di(heptadecan-9-yl) 15- |
| | methyl-14, 17-dioxo-9,22- |
| | bis(6-oxo-6- |
| | (undecyloxy)hexyl)- |
| | 9,13,18,22- |
| | tetraazatriacontanedioate |
| | 6-[2-[4-[2-[bis [6-(2- |
| | hexyldecanoyloxy)hexyl] |
| | amino]ethylamino]-4-oxo- |
| | but-2-enoyl]amino]ethyl- |
| | [6-(2- |
| | hexyldecanoyloxy)hexyl] |
| | aminolhexyl 2- |
| | hexyldecanoate |
| | di(heptadecan-9-yl) 9,20- |
| | bis(8-((2- |
| | methylnonyl)oxy)-8- |
| | oxooctyl)-13,16-dioxo- |
| | 9,12,17,20- |
| | tetraazaoctacos-14- |
| | enedioate |
| | di(heptadecan-9-yl) 9,20- |
| | bis(7-((2- |
| | octyldecanoyl)oxy)heptyl)- |
| | 13,16-dioxo-9,12,17,20- |
| | tetraazaoctacos-14- |
| | enedioate |
| | di(heptadecan-9-yl) 9,20- |
| | bis (6-((2- |
| | methylundecyl)oxy)-6- |
| | oxohexyl)-13,16-dioxo- |
| | 9,12,17,20- |
| | tetraazaoctacos-14- |
| | enedioate |
| | di(heptadecan-9-yl) 9,20- |
| | bis(5- |
| | (dodecanoyloxy)pentyl)- |
| | 13,16-dioxo-9,12,17,20- |
| | tetraazaoctacos-14- |
| | enedioate |
| | di(heptadecan-9-yl) |
| | 14,17-dioxo-9,22-bis(6- |
| | oxo-6-(undecyloxy)hexyl)- |
| | 9,13,18,22- |
| | tetraazatriacont-15- |
| | enedioate |
| | 6-[2-[4-[2-[bis[6-(2- |
| | hexyldecanoyloxy)hexylJa |
| | mino]ethylamino]-4-oxo- |
| | but-2-enoyl]amino]ethyl- |
| | [6-(2- |
| | hexyldecanoyloxy)hexyl]a |
| | mino]hexyl 2- |
| | hexyldecanoate |
| | di(heptadecan-9-yl) 9,19- |
| | bis (8-((2- |
| | methylnonyl)oxy)-8- |
| | oxooctyl)-13, 15-dioxo- |
| | 9,12,16,19- |
| | tetraazaheptacosanedioate |
| | di(heptadecan-9-yl) 9,19- |
| | bis (7-((2- |
| | octyldecanoyl)oxy)heptyl)- |
| | 13,15-dioxo-9,12,16,19- |
| | tetraazaheptacosanedioate |
| | di(heptadecan-9-yl) 9,19- |
| | bis (6-((2- |
| | methylundecyl)oxy)-6- |
| | oxohexyl)-13, 15-dioxo- |
| | 9,12,16,19- |
| | tetraazaheptacosanedioate |
| | di(heptadecan-9-yl) |
| | 14,16-dioxo-9,21-bis(6- |
| | oxo-6-(undecyloxy)hexyl)- |
| | 9,13,17,21- |
| | tetraazanonacosanedioate |
| | 6-[2-[5-[2-[bis[6-(2- |
| | hexyldecanoyloxy)hexyl] |
| | amino]ethylamino]-5-oxo- |
| | pentanoyl]amino]ethyl-[6- |
| | (2- |
| | hexyldecanoyloxy)hexyl] |
| | amino]hexyl 2- |
| | hexyldecanoate |
| | di(heptadecan-9-yl) 9,21- |
| | bis (8-((2- |
| | methylnonyl)oxy)-8- |
| | oxooctyl)-13, 17-dioxo- |
| | 9,12,18,21- |
| | tetraazanonacosanedioaet |
| | di(heptadecan-9-yl) 9,21- |
| | bis(7-((2- |
| | octyldecanoyl)oxy)heptyl)- |
| | 13,17-dioxo-9,12,18,21- |
| | tetraazanonacosanedioate |
| | di(heptadecan-9-yl) 9,21- |
| | bis (6-((2- |
| | methylundecyl)oxy)-6- |
| | oxohexyl)-13,17-dioxo- |
| | 9,12,18,21- |
| | tetraazanonacosanedioate |
| | di(heptadecan-9-yl) |
| | 13,17-dioxo-9,21-bis(6- |
| | oxo-6-(tridecan-3- |
| | yloxy)hexyl)-9,12,18,21- |
| | tetraazanonacosanedioate |
| | di(heptadecan-9-yl) 9,21- |
| | bis(5- |
| | (dodecanoyloxy)pentyl)- |
| | 13,17-dioxo-9,12,18,21- |
| | tetraazanonacosanedioat |
| | e |
| | di(heptadecan-9-yl) |
| | 14,18-dioxo-9,23-bis(6- |
| | oxo-6-(undecyloxy)hexyl)- |
| | 9,13,19,23- |
| | tetraazahentriacontanedioate |
| | 6-[2-[2-[2-[2-[bis [6-(2- |
| | hexyldecanoyloxy)hexyl] |
| | amino]ethylamino]-2-oxo- |
| | ethyl]-(2-pyrrolidin-1- |
| | ylacetyl)amino]acetyl] |
| | amino]ethyl-[6-(2- |
| | hexyldecanoyloxy)hexyl] |
| | amino]hexyl 2- |
| | hexyldecanoate |
| | di(heptadecan-9-yl) 9,21- |
| | bis(8-(heptadecan-9- |
| | yloxy)-8-oxooctyl)-13,17- |
| | dioxo-15-(2-(pyrrolidin-1- |
| | yl)acetyl)-9,12,15,18,21- |
| | pentaazanonacosanedioate |
| | di(heptadecan-9-yl) 9,21- |
| | bis(8-((2- |
| | methylnonyl)oxy)-8- |
| | oxooctyl)-13, 17-dioxo-15- |
| | (2-(pyrrolidin-1-yl)acetyl)- |
| | 9,12,15,18,21- |
| | pentaazanonacosanedioate |
| | di(heptadecan-9-yl) 9,21- |
| | bis(7-((2- |
| | octyldecanoyl)oxy)heptyl)- |
| | 13,17-dioxo-15-(2- |
| | (pyrrolidin-1-yl)acetyl)- |
| | 9,12,15,18,21- |
| | pentaazanonacosanedioate |
| | di(heptadecan-9-yl) 9,21- |
| | bis (6-((2- |
| | methylundecyl)oxy)-6- |
| | oxohexyl)-13,17-dioxo- |
| | 15-(2-(pyrrolidin-1- |
| | yl)acetyl)-9,12,15,18,21- |
| | pentaazanonacosanedioate |
| | di(heptadecan-9-yl) |
| | 13,17-dioxo-9,21-bis(6- |
| | oxo-6-(tridecan-3- |
| | yloxy)hexyl)-15-(2- |
| | (pyrrolidin-1-yl)acetyl)- |
| | 9,12,15,18,21- |
| | pentaazanonacosanedioate |
| | di(heptadecan-9-yl) 9,21- |
| | bis(5- |
| | (dodecanoyloxy)pentyl)- |
| | 13,17-dioxo-15-(2- |
| | (pyrrolidin-1-yl)acetyl)- |
| | 9,12,15,18,21- |
| | pentaazanonacosanedioate |
| | di(heptadecan-9-yl) |
| | 14,18-dioxo-9,23-bis(6- |
| | oxo-6-(undecyloxy)hexyl)- |
| | 16-(2-(pyrrolidin-1- |
| | yl)acetyl)-9,13,16,19,23- |
| | pentaazahentriacontanedioate |
| | ((piperazine-1,4- |
| | diylbis(ethane-2,1- |
| | diyl)) bis(azanetriyl)) |
| | tetrakis(hexane-6,1-diyl) |
| | tetrakis(2- |
| | hexyldecanoate) |
| | tetra(heptadecan-9-yl) |
| | 8,8′,8″,8″-((piperazine- |
| | 1,4-diylbis(ethane-2,1- |
| | diyl)) bis(azanetriyl)) |
| | tetraoctanoate |
| | di(heptadecan-9-yl) 8,8′- |
| | ((piperazine-1,4- |
| | diylbis(ethane-2,1- |
| | diyl)) bis ((8-((2- |
| | methylnonyl)oxy)-8- |
| | oxooctyl)azanediyl)) |
| | dioctanoate |
| | ((piperazine-1,4- |
| | diylbis(ethane-2,1- |
| | diyl)) bis((8-(heptadecan- |
| | 9-yloxy)-8- |
| | oxooctyl)azanediyl))bis |
| | (heptane-7,1-diyl) bis(2- |
| | octyldecanoate) |
| | di(heptadecan-9-yl) 8,8′- |
| | ((piperazine-1,4- |
| | diylbis(ethane-2,1- |
| | diyl)) bis ((6-((2- |
| | methylundecyl)oxy)-6- |
| | oxohexyl)azanediyl)) |
| | dioctanoate |
| | di(heptadecan-9-yl) 8,8′- |
| | ((piperazine-1,4- |
| | diylbis(propane-3,1- |
| | diyl))bis((6-oxo-6- |
| | (undecyloxy)hexyl) |
| | azanediyl))dioctanoate |

### Ionizable Lipid Compounds ii)

In some embodiments, the ionizable lipid is represented by the following formula II:

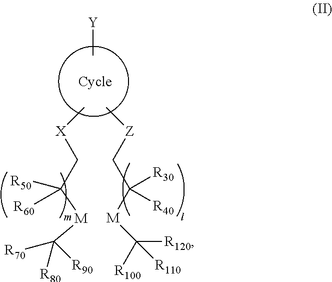

a pharmaceutically acceptable salt thereof, or a stereoisomer of any of the foregoing, wherein:

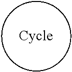

is cyclic or heterocyclic moiety;
Y is alkyl, hydroxy, hydroxyalkyl or

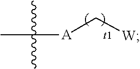

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl.

In some embodiments, Y is hydroxyl or

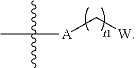

In some embodiments,

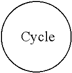

is selected from pyrrolidine, piperidine, piperazine, cyclohexane, cyclopentane, tetrahydrofuran, tetrahydropyran, morpholine, and dioxane. In some embodiments,

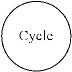

is selected from the group consisting of

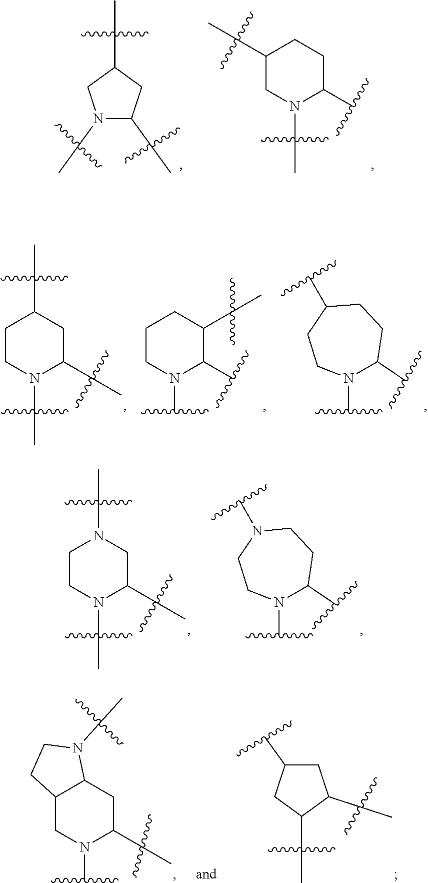

In some embodiments, the ionizable lipid is represented by formula:

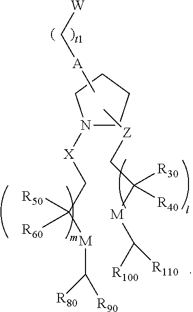

All the variables in this formula have been defined and exemplified as those described in the above embodiments.

In some embodiments, the ionizable lipid is represented by formula:

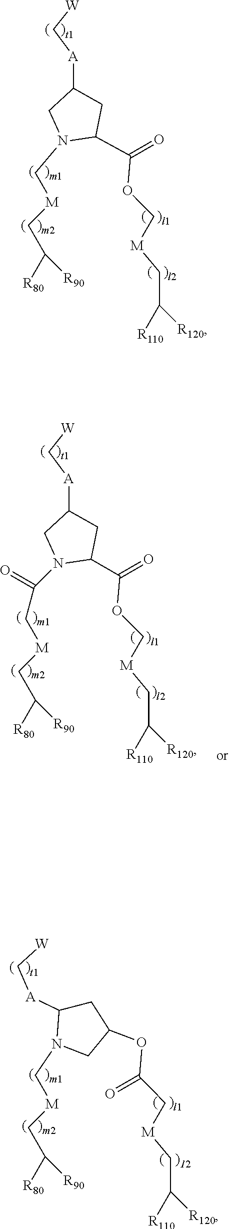

wherein:
A is absent, —O—, —N(R⁷)—, —O-alkylene-, -alkylene-O—, —OC(O)—, —C(O)O—,
—N(R⁷)C(O)—, —C(O)N(R⁷)—, —N(R⁷)C(O)N(R⁷)—, —S—, —S—S—, or a bivalent heterocycle;
each R⁷ is independently H, alkyl, alkenyl, cycloalkyl, hydroxy, hydroxyalkyl, or aminoalkyl;
t1 is an integer from 0 to 10;
W is hydroxyl, substituted or unsubstituted hydroxyalkyl, substituted or unsubstituted amino, substituted or unsubstituted aminocarbonyl, or substituted or unsubstituted heterocyclyl or heteroaryl;
each M is independently a biodegradable moiety;
each m1 is independently an integer from 3 to 6,
each l1 is independently an integer from 4 to 8,
m2 and l2 are each independently an integer from 0 to 3,
R₈₀ and R₉₀ are each independently unsubstituted C₅-C₈ alkyl; or R₈₀ is H or unsubstituted C₁-C₄ alkyl, and R₉₀ is unsubstituted C₅-C₁₁ alkyl; and
R₁₁₀ and R₁₂₀ are each independently unsubstituted C₅-C₈ alkyl; or R₁₁₀ is H or unsubstituted C₁-C₄ alkyl, and R₁₂₀ is unsubstituted C₅-C₁₁ alkyl. All the other variables in these formulas have been defined and exemplified as those described in the above embodiments. In some embodiments, in these formulas, R₈₀ is H or unsubstituted C₁-C₂ alkyl, and R₉₀ is unsubstituted C₆-C₁₀ alkyl; and R₁₁₀ and R₁₂₀ are each independently unsubstituted C₅-C₈ alkyl. In some embodiments, R₈₀, R₉₀, R₁₁₀, and R₁₂₀ are each independently unsubstituted C₅-C₈ alkyl.

In some embodiments, in the above formulas, A is absent, —O—, —N(R⁷)—, N(R⁷)C(O)—,

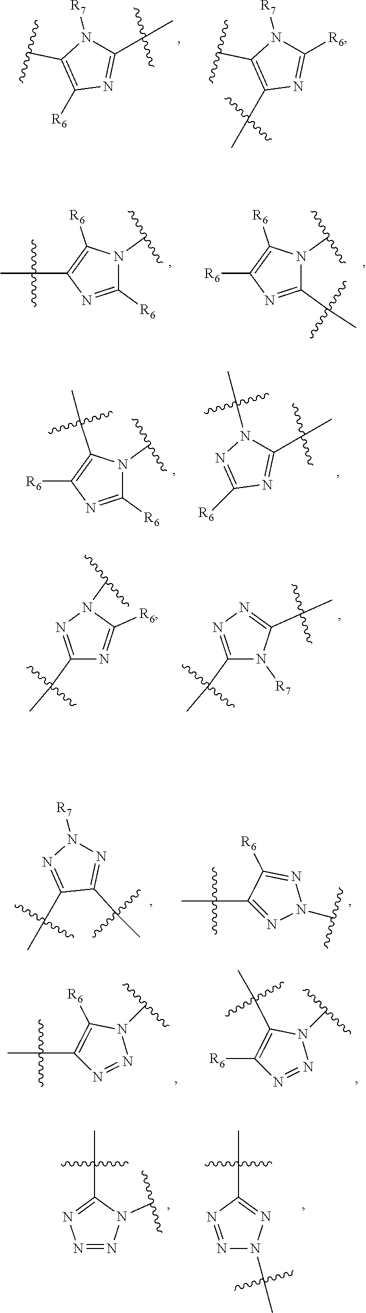

—OC(O)—, or —C(O)O—, wherein R⁶ is independently H, alkyl, hydroxyl, hydroxyalkyl, amino, aminoalkyl, thiol, thiolalkyl, or N⁺(R⁷)₃-alkylene-Q-; and R⁷ is H or C₁-C₃ alkyl.

In some embodiments, in the above formulas, t1 is 0, 1, 2, 3 or 4; and t is 0, 1, or 2.

In some embodiments, in the above formulas, W is hydroxyl, hydroxyalkyl, or one of the following:

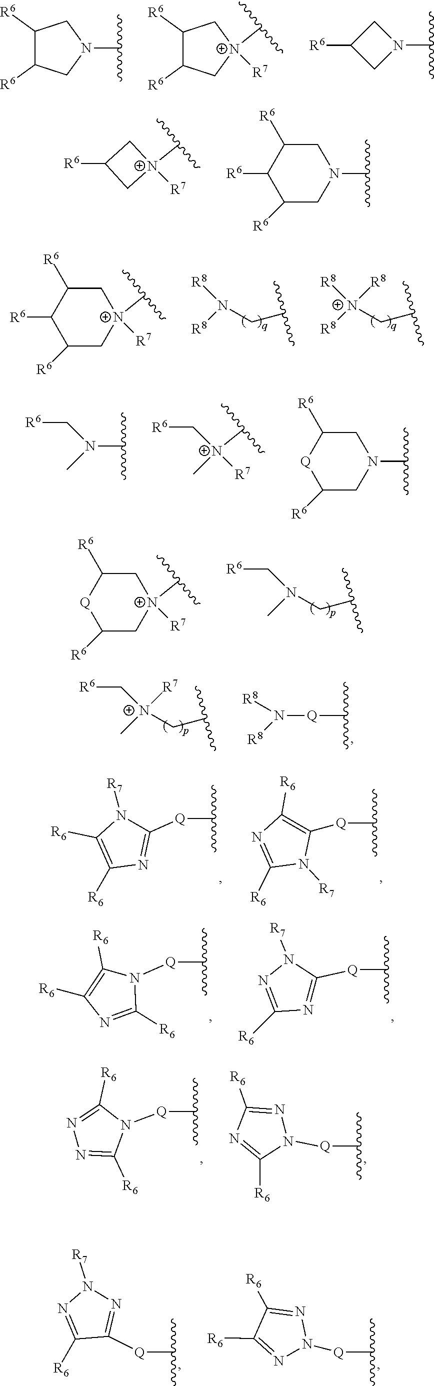

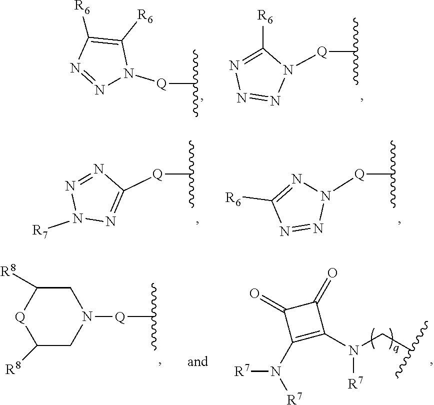

wherein:
each Q is independently absent, —O—, —C(O)—, —C(S)—, —C(O)O—, —C(R⁷)₂—, —C(O)N(R⁷)—, —C(S)N(R⁷)—, or —N(R⁷)—;
each R⁶ is independently H, alkyl, hydroxyl, hydroxyalkyl, alkoxy, amino, aminoalkyl, alkylamino, thiol, thiolalkyl, or N⁺(R⁷)₃-alkylene-Q-;
each R⁸ is independently H, alkyl, hydroxyalkyl, amino, aminoalkyl, thiol, or thiolalkyl, or two R⁸ together with the nitrogen atom may form a ring;
each q is independently 0, 1, 2, 3, 4, or 5; and
each p is independently 0, 1, 2, 3, 3, 4, or 5.

In some embodiments, in the above formulas,
X is absent, —O—, or —C(O)—;
Z is —O—, —C(O)O—, or —OC(O)—;
M is —OC(O)— or —C(O)O—;
Y or

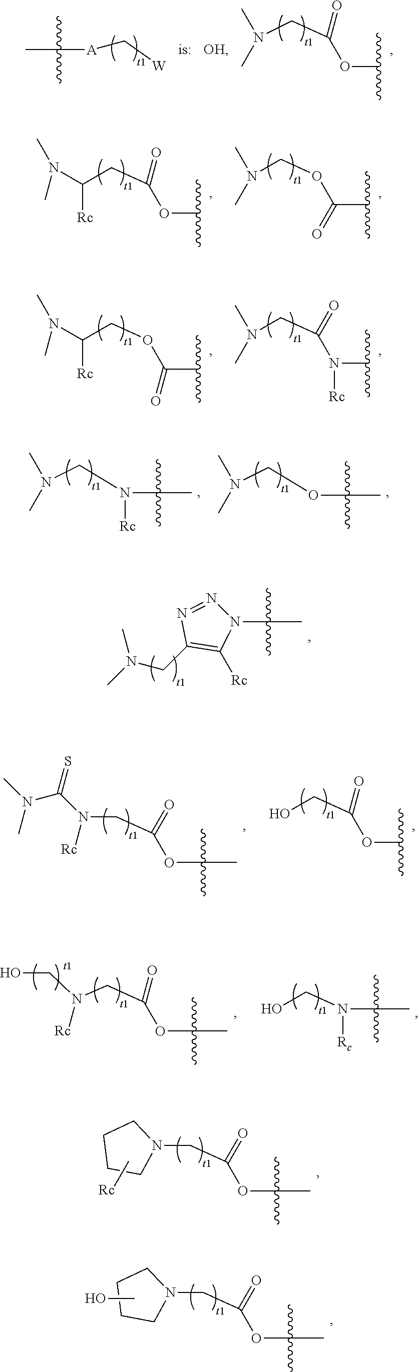

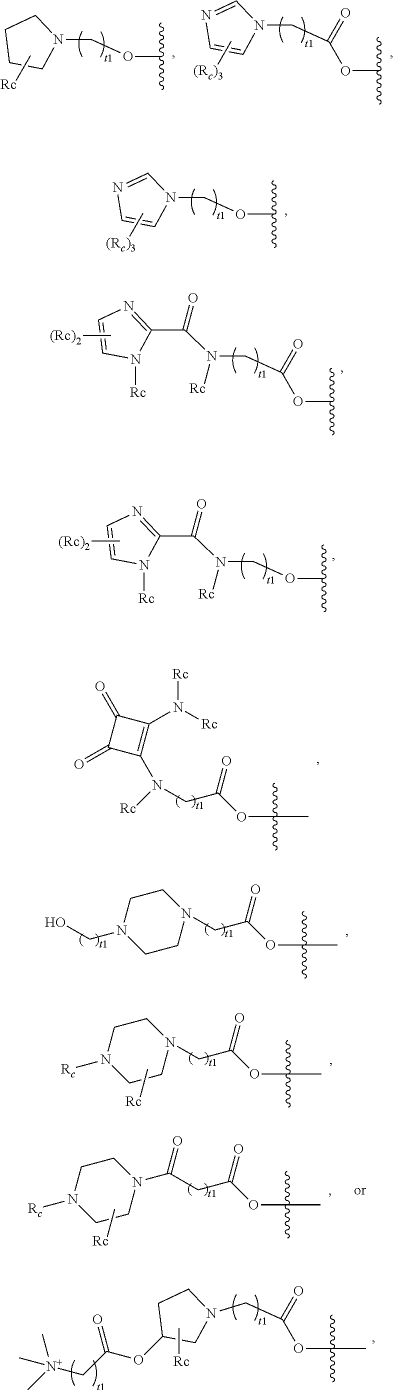

each R^{c} is independently H or C₁-C₃ alkyl;
each t1 is independently 1, 2, 3, or 4;
each of R₃₀, R₄₀, R₅₀, and R₆₀ is H or C₁-C₄ branched or unbranched alkyl;
R₇₀ is H; and each of R₈₀ and R₉₀ is independently H or C₁-C₁₂ branched or unbranched alkyl;
R₁₀₀ is H; and each of R₁₁₀ and R₁₂₀ is independently H or C₁-C₁₂ branched or unbranched alkyl, provided that at least one of R₈₀ and R₉₀ is not H, and at least one of R₁₁₀ and R₁₂₀ is not H;
l is from 3 to 7; and
m is from 1 to 5.

In some embodiments, in the above formulas, Y or

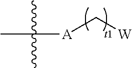

### is: OH,

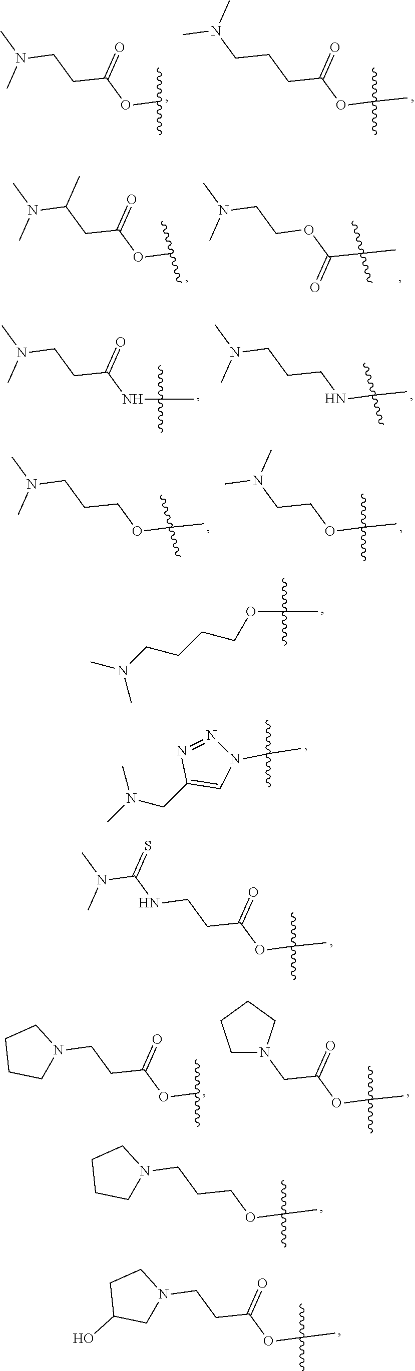

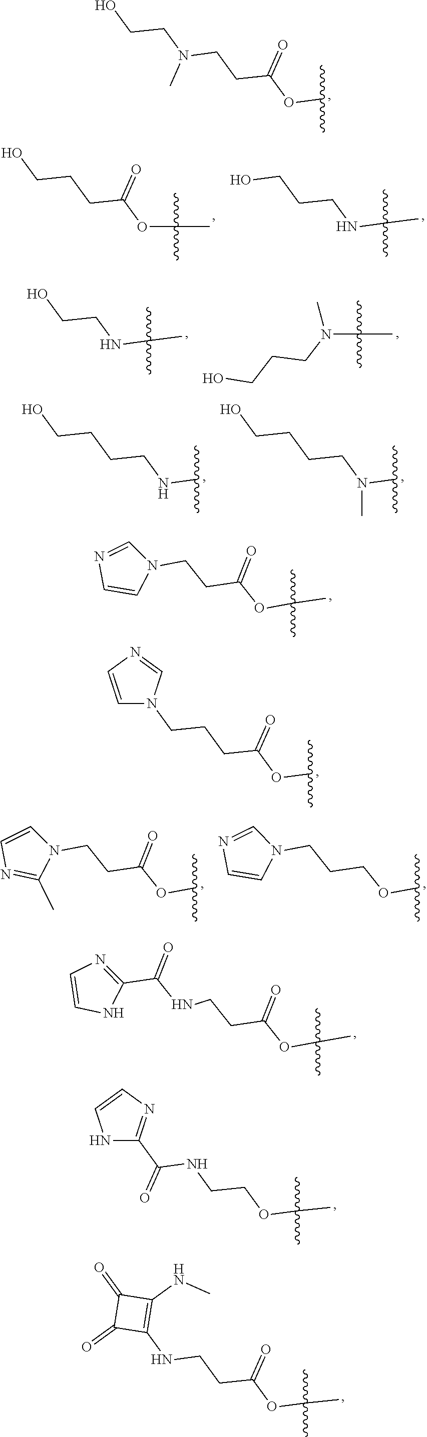

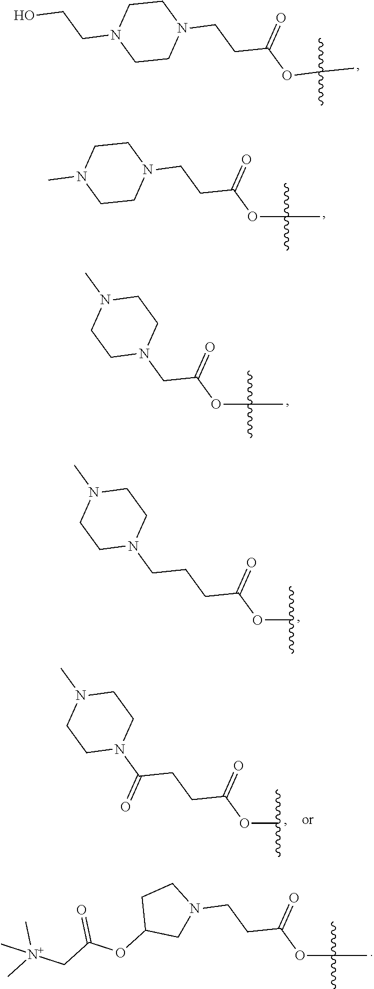 [SMILES: *OC(=O)CCN1CCN(CCO)CC1 ; *OC(=O)CCN1CCN(C)CC1 ; *OC(=O)CN1CCN(C)CC1 ; *OC(=O)CCCN1CCN(C)CC1 ; *OC(=O)CCC(=O)N1CCN(C)CC1 ; *OC(=O)CCN1CCC(OC(=O)C[N+](C)(C)C)C1]

More embodiments of the ionizable lipid of formula (II) in the Ionizable lipid compounds group ii), may be found in PCT Application No. PCT/US23/16300, filed on Mar. 24, 2023, the content of which is incorporated herein by reference in its entirety. In particular, all the ionizable lipids of formulas (I), (IA-1), (IA-2), (IIA)-(IIC), (IIA-1), (IIIA)-(IIIIE), (IIIC-1), (IVA-1)-(IVA-3), (IVC-1)-(IVC-2), (VA-1)-(VA-9), (VC-1)-(VC-6) of PCT Application No. PCT/US23/16300 are suitable for use as the ionizable lipids in this disclosure, and are incorporated herein by reference in its entirety.

Certain exemplary ionizable lipid compounds disclosed herein are set forth in Table II below.

TABLE II
Exemplary ionizable lipid compounds.

| Lipid | |
|---|---|
| No. | |
| 2310 | 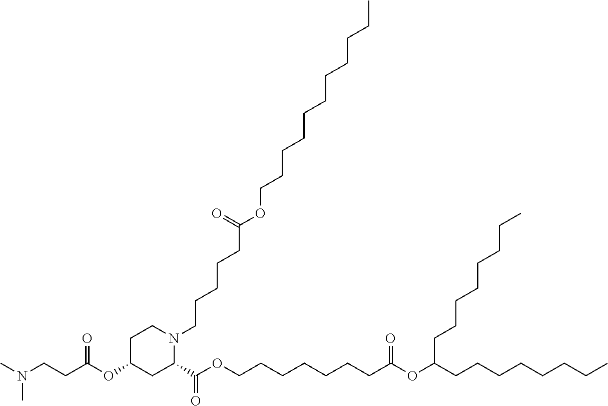 [SMILES: CCCCCCCCCCCOC(=O)CCCCCN1CC[C@@H](OC(=O)CCN(C)C)C[C@H]1C(=O)OCCCCCCCC(=O)OC(CCCCCCCC)CCCCCCCC] |
| 2309 | 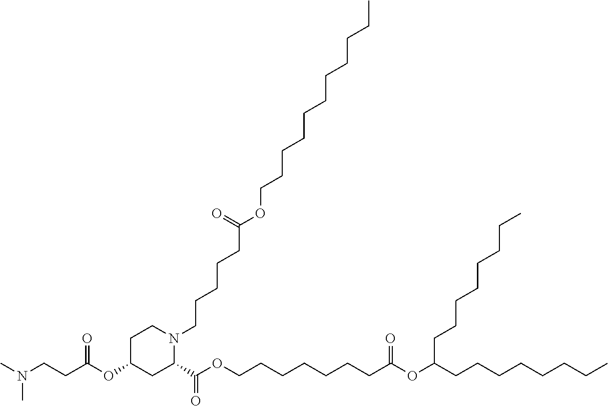 [SMILES: CCCCCCCCCCCOC(=O)CCCCCN1CC[C@@H](OC(=O)CCN(C)C)C[C@H]1C(=O)OCCCCCCCC(=O)OC(CCCCCCCC)CCCCCCCC] |
| 2308 | 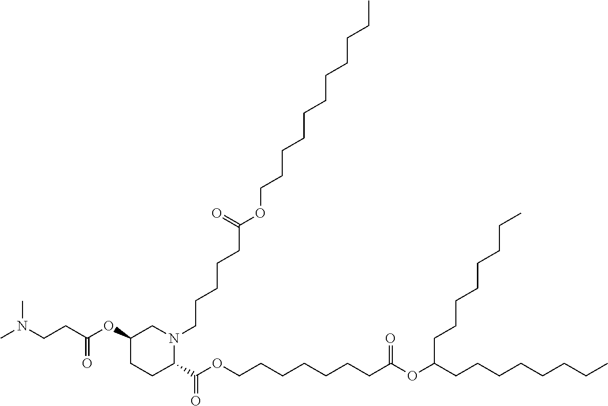 [SMILES: CCCCCCCCCCCOC(=O)CCCCCN1C[C@H](OC(=O)CCN(C)C)CC[C@H]1C(=O)OCCCCCCCC(=O)OC(CCCCCCCC)CCCCCCCC] |
| 2307 | 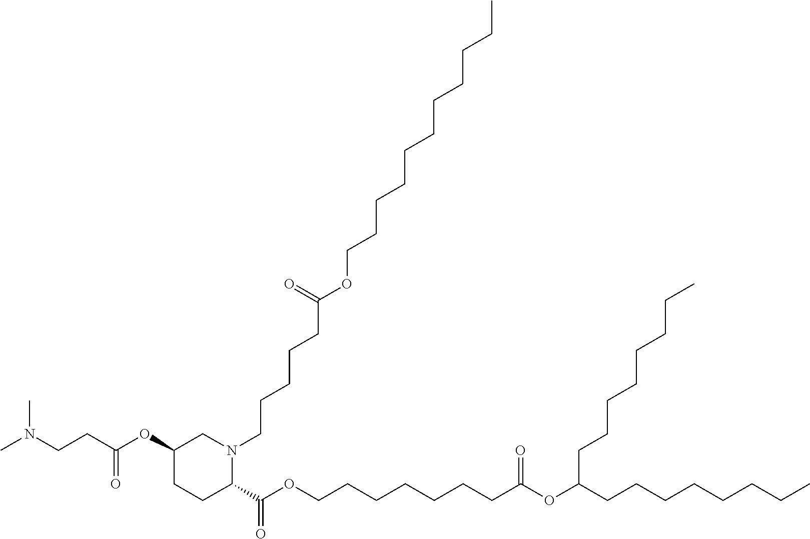 [SMILES: CCCCCCCCCCCOC(=O)CCCCCN1C[C@H](OC(=O)CCN(C)C)CC[C@H]1C(=O)OCCCCCCCC(=O)OC(CCCCCCCC)CCCCCCCC] |
| 2306 | 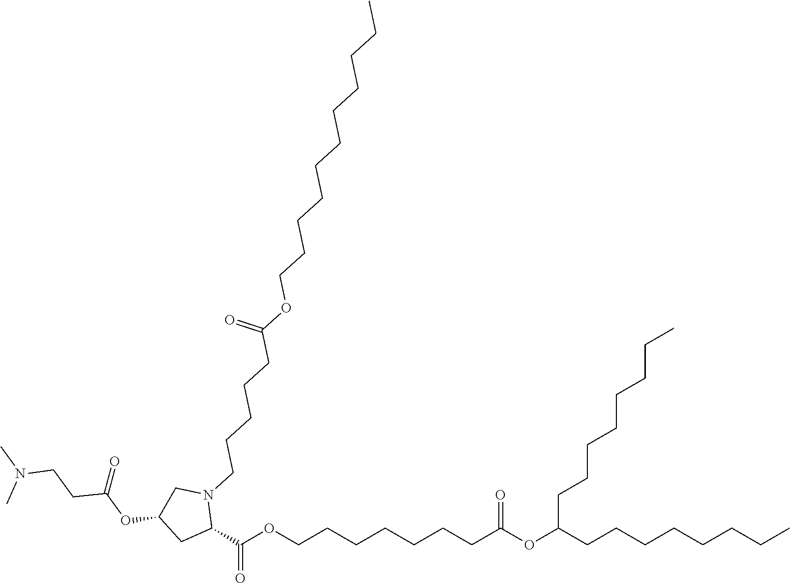 [SMILES: CCCCCCCCCCCOC(=O)CCCCCN1C[C@@H](OC(=O)CCN(C)C)C[C@H]1C(=O)OCCCCCCCC(=O)OC(CCCCCCCC)CCCCCCCC] |
| 2305 | 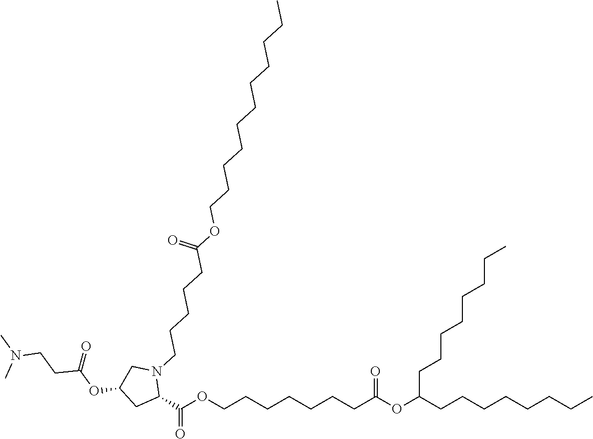 [SMILES: CCCCCCCCCCCOC(=O)CCCCCN1C[C@@H](OC(=O)CCN(C)C)C[C@H]1C(=O)OCCCCCCCC(=O)OC(CCCCCCCC)CCCCCCCC] |
| 2304 | 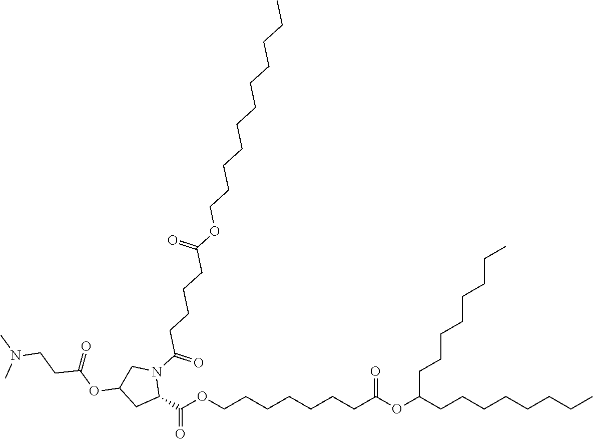 [SMILES: CCCCCCCCCCCOC(=O)CCCCC(=O)N1CC(OC(=O)CCN(C)C)C[C@H]1C(=O)OCCCCCCCC(=O)OC(CCCCCCCC)CCCCCCCC] |
| 2298 | 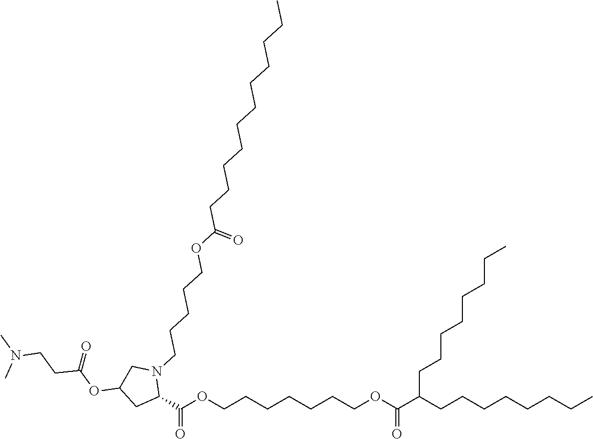 [SMILES: CCCCCCCCCCCC(=O)OCCCCCN1CC(OC(=O)CCN(C)C)C[C@H]1C(=O)OCCCCCCCOC(=O)C(CCCCCCCC)CCCCCCCC] |
| 2297 | 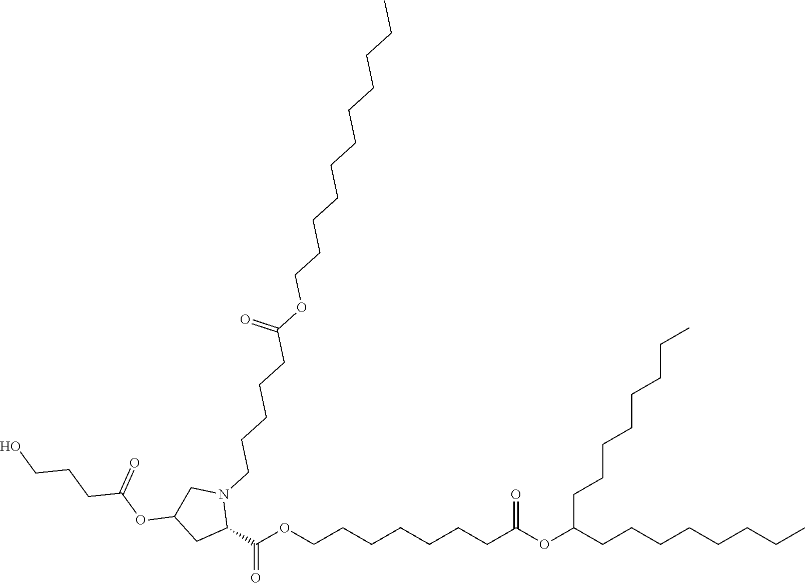 [SMILES: CCCCCCCCCCCOC(=O)CCCCCN1CC(OC(=O)CCCO)C[C@H]1C(=O)OCCCCCCCC(=O)OC(CCCCCCCC)CCCCCCCC] |
| 2296 | 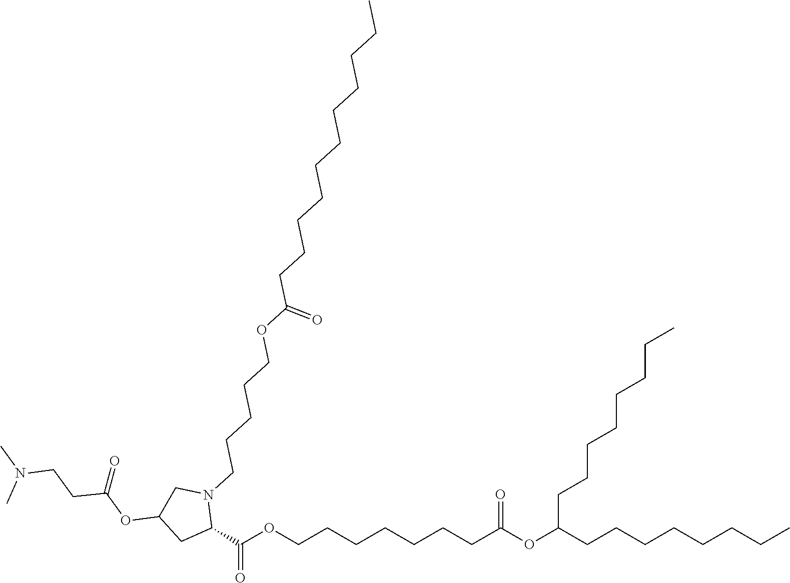 [SMILES: CCCCCCCCCCCC(=O)OCCCCCN1CC(OC(=O)CCN(C)C)C[C@H]1C(=O)OCCCCCCCC(=O)OC(CCCCCCCC)CCCCCCCC] |
| 2295 | 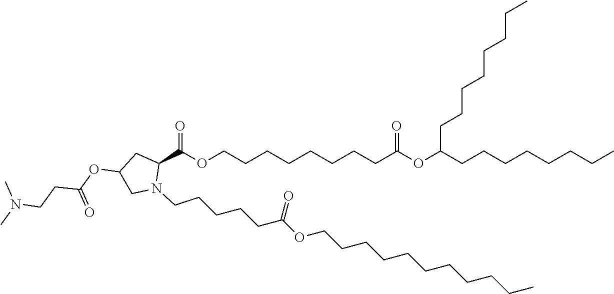 [SMILES: CCCCCCCCCCCOC(=O)CCCCCN1CC(OC(=O)CCN(C)C)C[C@H]1C(=O)OCCCCCCCCC(=O)OC(CCCCCCCC)CCCCCCCC] |
| 2294 | 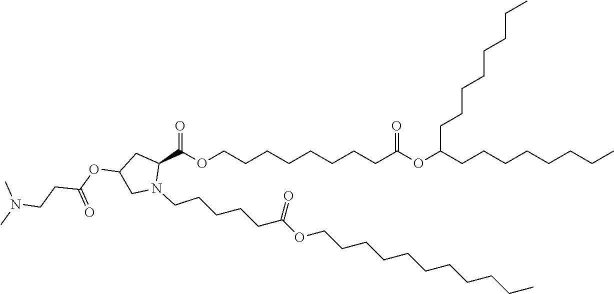 [SMILES: CCCCCCCCCCCOC(=O)CCCCCN1CC(OC(=O)CCN(C)C)C[C@H]1C(=O)OCCCCCCCCC(=O)OC(CCCCCCCC)CCCCCCCC] |
| 2293 | 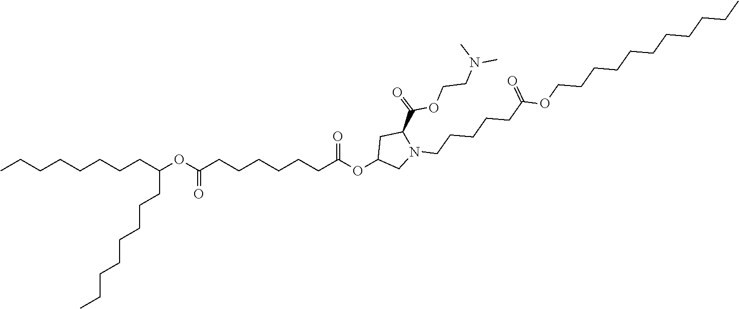 [SMILES: CCCCCCCCCCCOC(=O)CCCCCN1CC(OC(=O)CCCCCCC(=O)OC(CCCCCCCC)CCCCCCCC)C[C@H]1C(=O)OCCN(C)C] |
| 2292 | 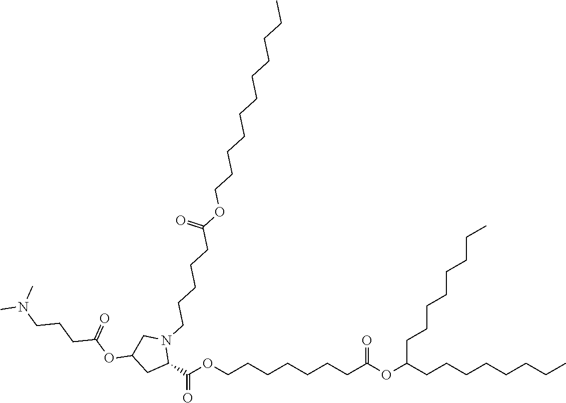 [SMILES: CCCCCCCCCCCOC(=O)CCCCCN1CC(OC(=O)CCCN(C)C)C[C@H]1C(=O)OCCCCCCCC(=O)OC(CCCCCCCC)CCCCCCCC] |
| 2291 | 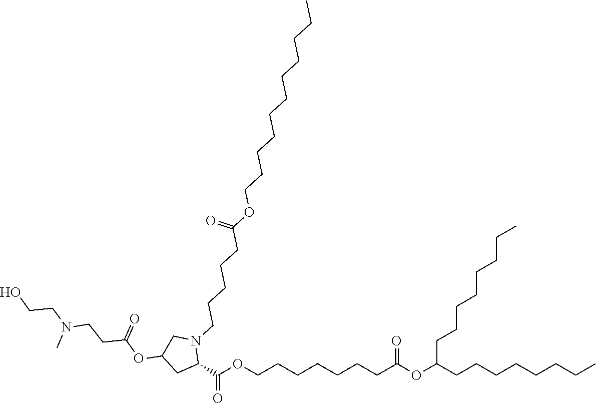 [SMILES: CCCCCCCCCCCOC(=O)CCCCCN1CC(OC(=O)CCN(C)CCO)C[C@H]1C(=O)OCCCCCCCC(=O)OC(CCCCCCCC)CCCCCCCC] |
| 2290 | 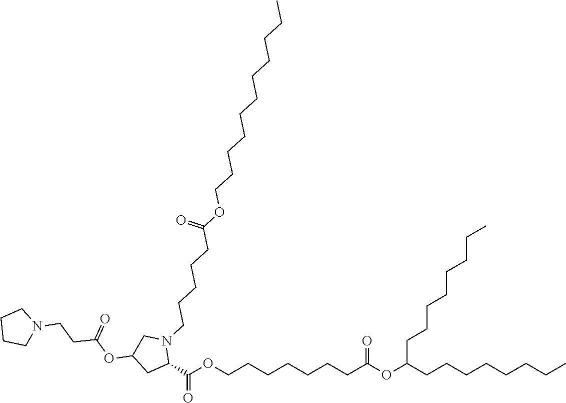 [SMILES: CCCCCCCCCCCOC(=O)CCCCCN1CC(OC(=O)CCN2CCCC2)C[C@H]1C(=O)OCCCCCCCC(=O)OC(CCCCCCCC)CCCCCCCC] |
| 2270 | 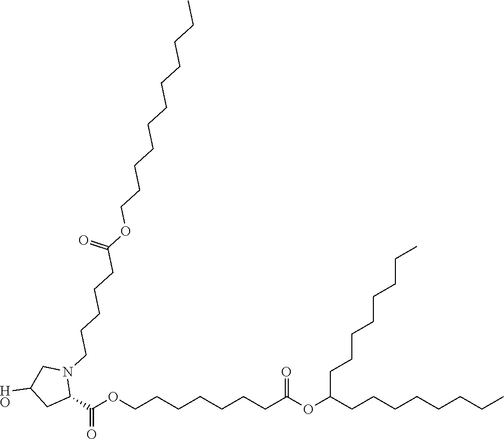 [SMILES: CCCCCCCCCCCOC(=O)CCCCCN1CC(O)C[C@H]1C(=O)OCCCCCCCC(=O)OC(CCCCCCCC)CCCCCCCC] |
| 2260 | 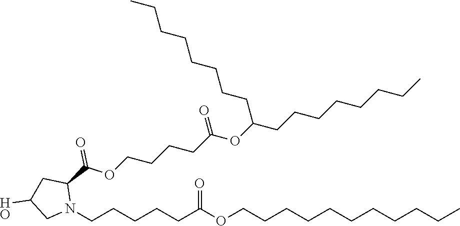 [SMILES: CCCCCCCCCCCOC(=O)CCCCCN1CC(O)C[C@H]1C(=O)OCCCCC(=O)OC(CCCCCCCC)CCCCCCCC] |
| 2240 | 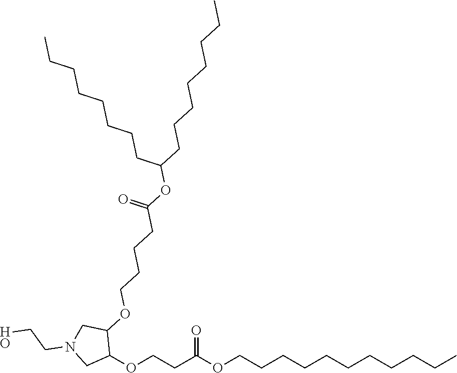 [SMILES: CCCCCCCCCCCOC(=O)CCOC1CN(CCO)CC1OCCCCC(=O)OC(CCCCCCCC)CCCCCCCC] |
| 2231 | 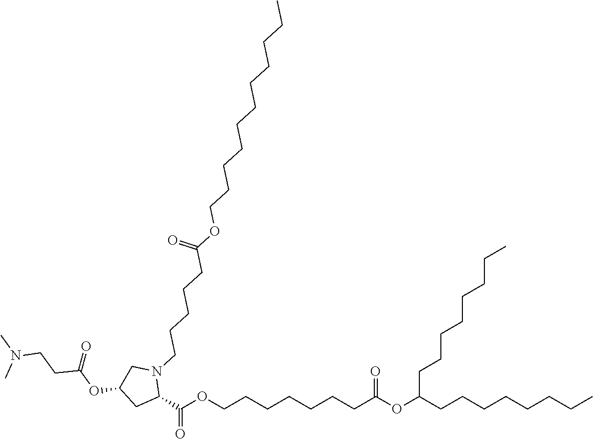 [SMILES: CCCCCCCCCCCOC(=O)CCCCCN1C[C@@H](OC(=O)CCN(C)C)C[C@H]1C(=O)OCCCCCCCC(=O)OC(CCCCCCCC)CCCCCCCC] |
| 2230 | 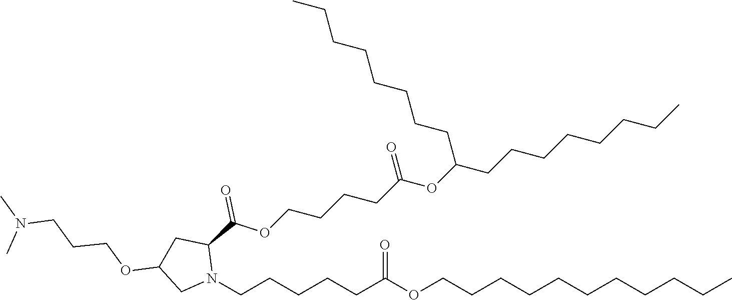 [SMILES: CCCCCCCCCCCOC(=O)CCCCCN1CC(OCCCN(C)C)C[C@H]1C(=O)OCCCCC(=O)OC(CCCCCCCC)CCCCCCCC] |
| 2329 | 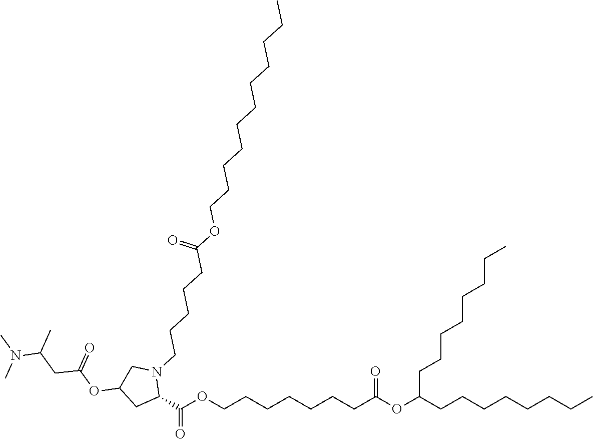 [SMILES: CCCCCCCCCCCOC(=O)CCCCCN1CC(OC(=O)CC(C)N(C)C)C[C@H]1C(=O)OCCCCCCCC(=O)OC(CCCCCCCC)CCCCCCCC] |
| 2336 | 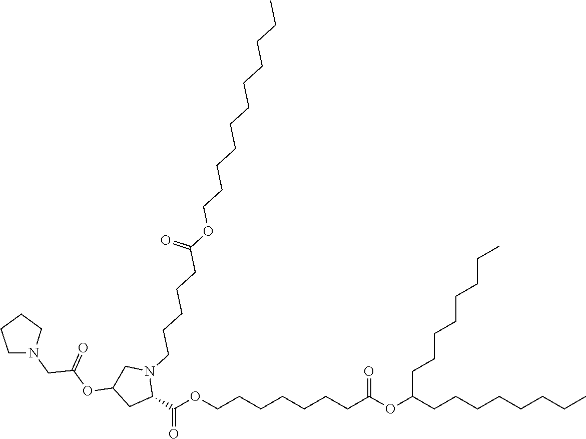 [SMILES: CCCCCCCCCCCOC(=O)CCCCCN1CC(OC(=O)CN2CCCC2)C[C@H]1C(=O)OCCCCCCCC(=O)OC(CCCCCCCC)CCCCCCCC] |
| 2337 | 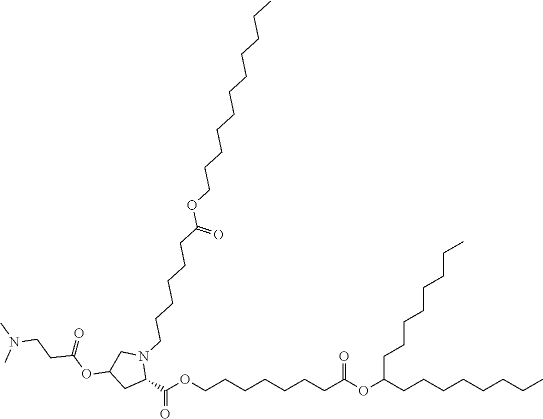 [SMILES: CCCCCCCCCCCOC(=O)CCCCCCN1CC(OC(=O)CCN(C)C)C[C@H]1C(=O)OCCCCCCCC(=O)OC(CCCCCCCC)CCCCCCCC] |
| 2338 | 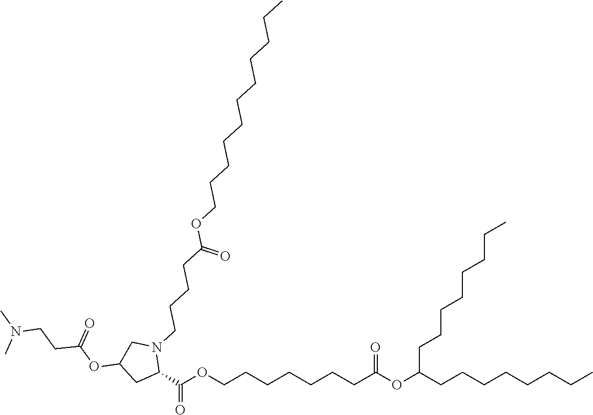 [SMILES: CCCCCCCCCCCOC(=O)CCCCN1CC(OC(=O)CCN(C)C)C[C@H]1C(=O)OCCCCCCCC(=O)OC(CCCCCCCC)CCCCCCCC] |
| 2339 | 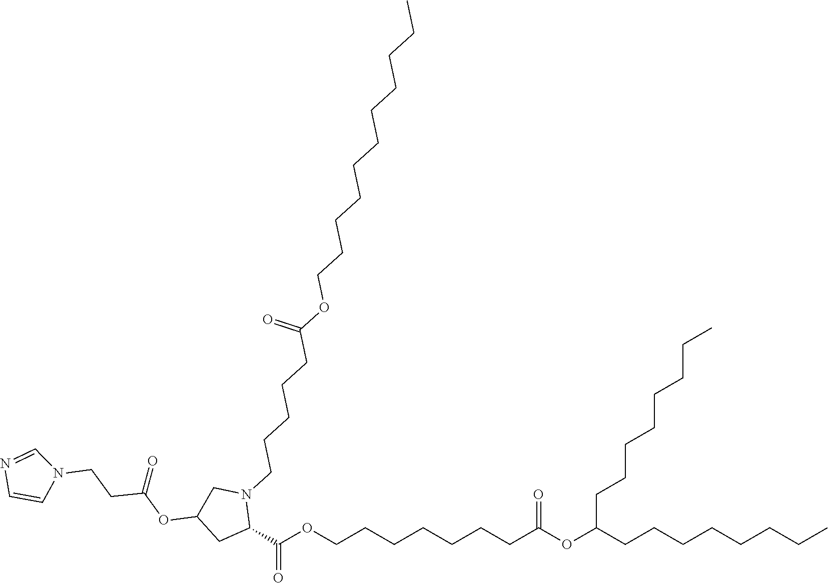 [SMILES: CCCCCCCCCCCOC(=O)CCCCCN1CC(OC(=O)CCn2ccnc2)C[C@H]1C(=O)OCCCCCCCC(=O)OC(CCCCCCCC)CCCCCCCC] |
| 2340 | 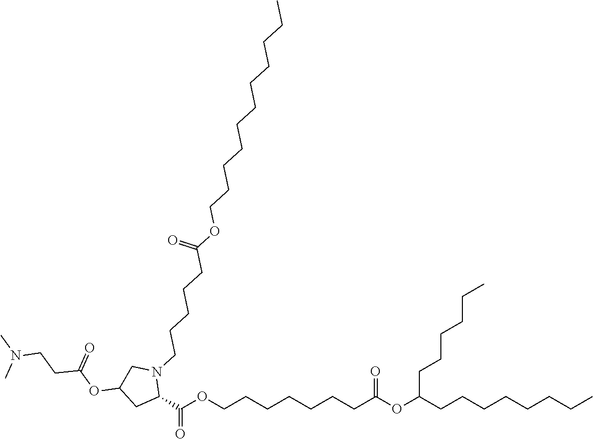 [SMILES: CCCCCCCCCCCOC(=O)CCCCCN1CC(OC(=O)CCN(C)C)C[C@H]1C(=O)OCCCCCCCC(=O)OC(CCCCCC)CCCCCCCC] |
| 2341 | 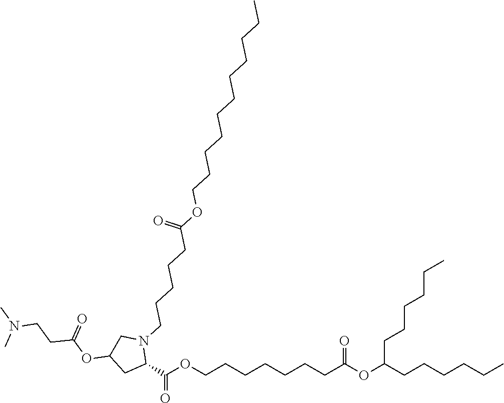 [SMILES: CCCCCCCCCCCOC(=O)CCCCCN1CC(OC(=O)CCN(C)C)C[C@H]1C(=O)OCCCCCCCC(=O)OC(CCCCCC)CCCCCC] |
| 2342 | 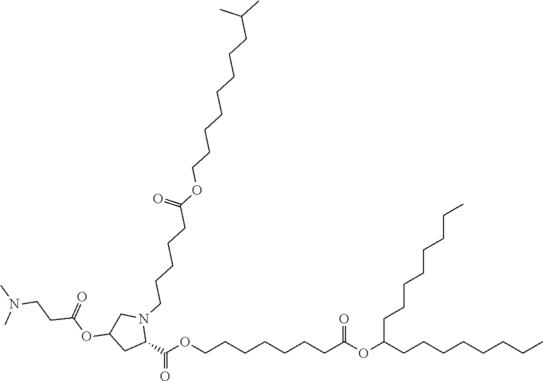 [SMILES: CCCCCCCCC(CCCCCCCC)OC(=O)CCCCCCCOC(=O)[C@@H]1CC(OC(=O)CCN(C)C)CN1CCCCCC(=O)OCCCCCCCCC(C)C] |
| 2343 | 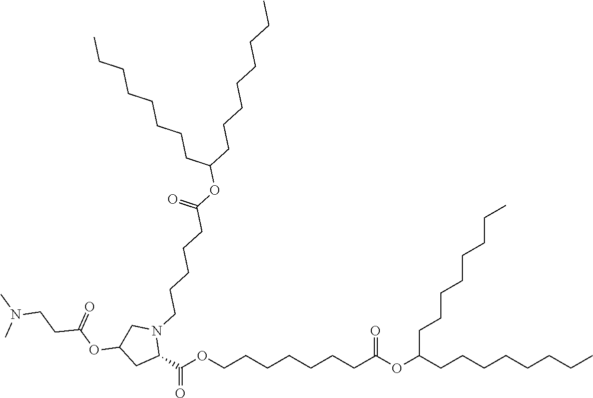 [SMILES: CCCCCCCCC(CCCCCCCC)OC(=O)CCCCCCCOC(=O)[C@@H]1CC(OC(=O)CCN(C)C)CN1CCCCCC(=O)OC(CCCCCCCC)CCCCCCCC] |
| 2344 | 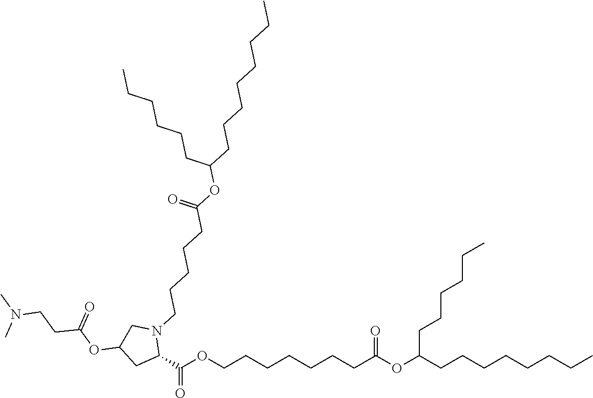 [SMILES: CCCCCCCCC(CCCCCC)OC(=O)CCCCCCCOC(=O)[C@@H]1CC(OC(=O)CCN(C)C)CN1CCCCCC(=O)OC(CCCCCC)CCCCCCCC] |
| 2345 | 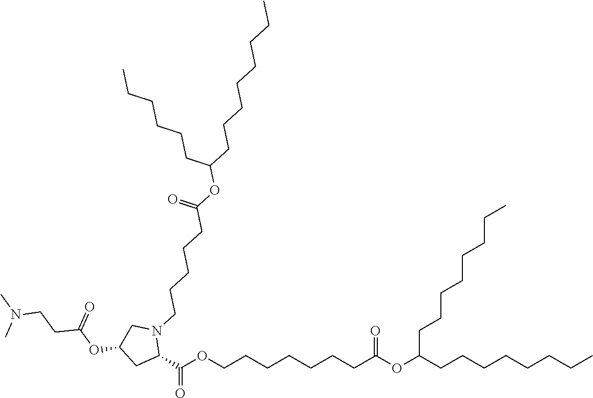 [SMILES: CCCCCCCCC(CCCCCC)OC(=O)CCCCCN1C[C@@H](OC(=O)CCN(C)C)C[C@H]1C(=O)OCCCCCCCC(=O)OC(CCCCCCCC)CCCCCCCC] |
| 2348 | 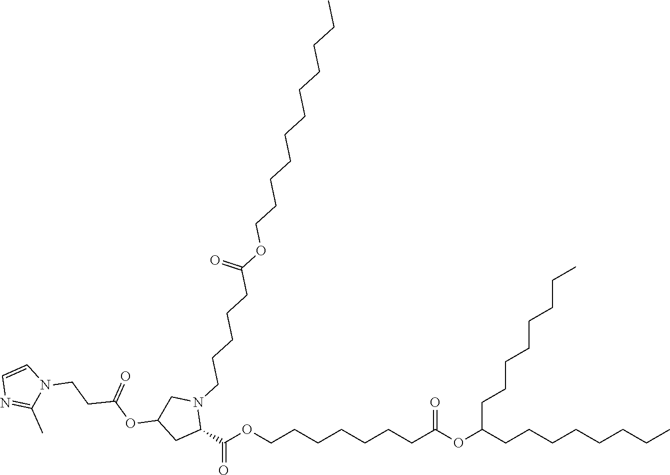 [SMILES: CCCCCCCCCCCOC(=O)CCCCCN1CC(OC(=O)CCn2ccnc2C)C[C@H]1C(=O)OCCCCCCCC(=O)OC(CCCCCCCC)CCCCCCCC] |
| 2349 | 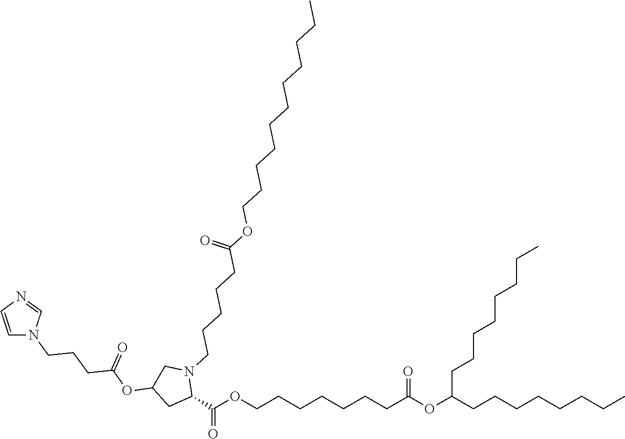 [SMILES: CCCCCCCCCCCOC(=O)CCCCCN1CC(OC(=O)CCCn2ccnc2)C[C@H]1C(=O)OCCCCCCCC(=O)OC(CCCCCCCC)CCCCCCCC] |
| 2352 | 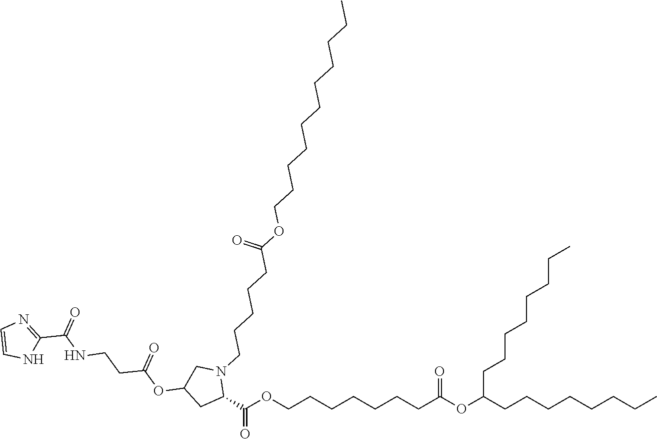 [SMILES: CCCCCCCCCCCOC(=O)CCCCCN1CC(OC(=O)CCNC(=O)c2ncc[nH]2)C[C@H]1C(=O)OCCCCCCCC(=O)OC(CCCCCCCC)CCCCCCCC] |
| 2371 | 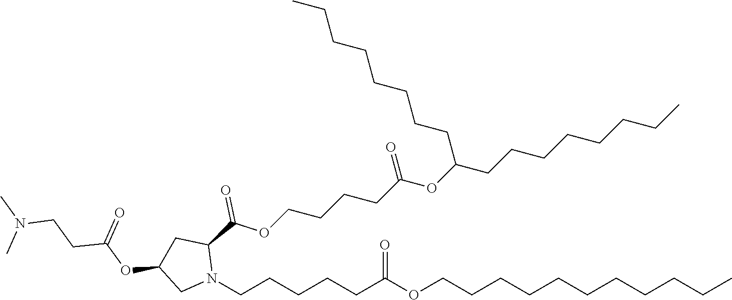 [SMILES: CCCCCCCCCCCOC(=O)CCCCCN1C[C@@H](OC(=O)CCN(C)C)C[C@H]1C(=O)OCCCCC(=O)OC(CCCCCCCC)CCCCCCCC] |
| 2372 | 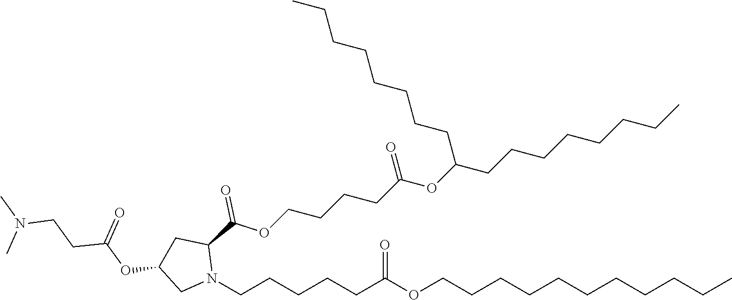 [SMILES: CCCCCCCCCCCOC(=O)CCCCCN1C[C@H](OC(=O)CCN(C)C)C[C@H]1C(=O)OCCCCC(=O)OC(CCCCCCCC)CCCCCCCC] |
| 2373 | 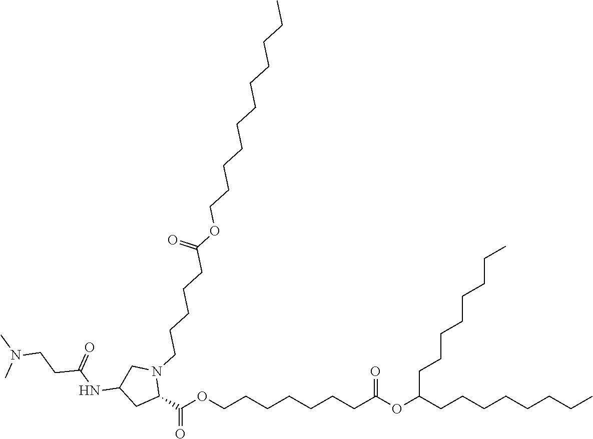 [SMILES: CCCCCCCCCCCOC(=O)CCCCCN1CC(NC(=O)CCN(C)C)C[C@H]1C(=O)OCCCCCCCC(=O)OC(CCCCCCCC)CCCCCCCC] |
| 2375 | 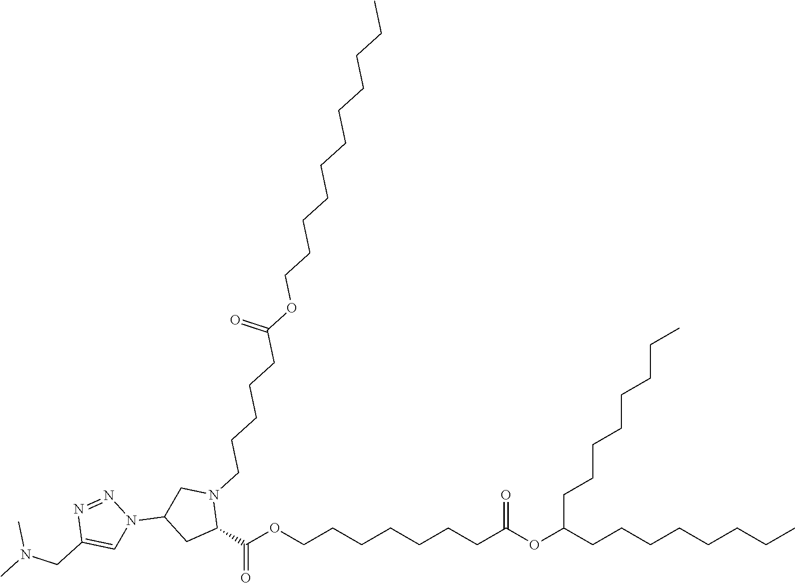 [SMILES: CCCCCCCCCCCOC(=O)CCCCCN1CC(n2cc(CN(C)C)nn2)C[C@H]1C(=O)OCCCCCCCC(=O)OC(CCCCCCCC)CCCCCCCC] |
| 2376 | 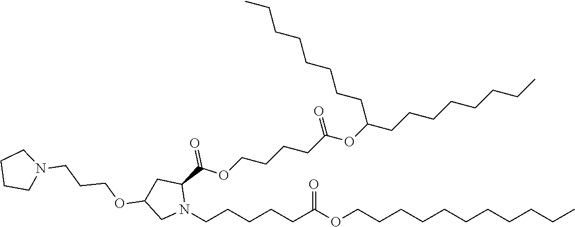 [SMILES: CCCCCCCCCCCOC(=O)CCCCCN1CC(OCCCN2CCCC2)C[C@H]1C(=O)OCCCCC(=O)OC(CCCCCCCC)CCCCCCCC] |
| 2377 | 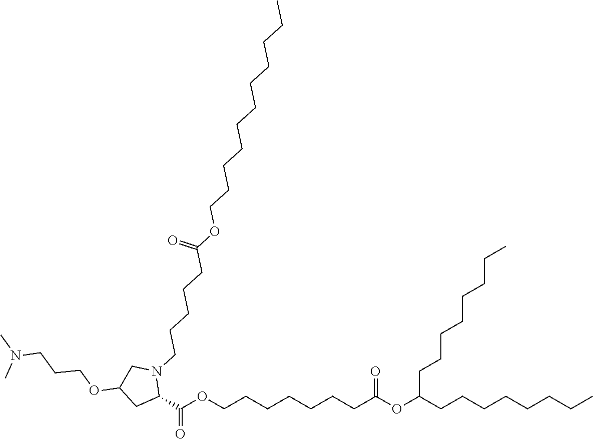 [SMILES: CCCCCCCCCCCOC(=O)CCCCCN1CC(OCCCN(C)C)C[C@H]1C(=O)OCCCCCCCC(=O)OC(CCCCCCCC)CCCCCCCC] |
| 2436 | 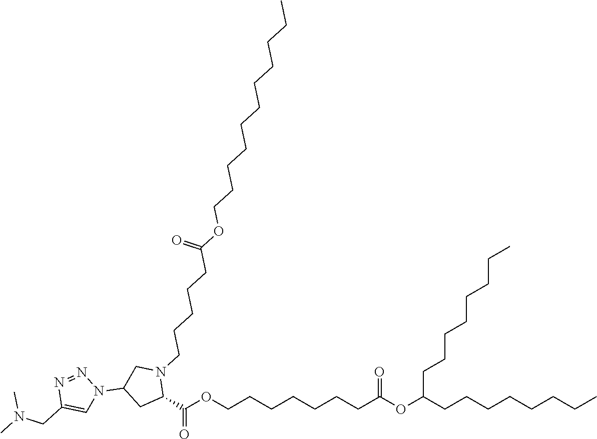 [SMILES: CCCCCCCCCCCOC(=O)CCCCCN1CC(n2cc(CN(C)C)nn2)C[C@H]1C(=O)OCCCCCCCC(=O)OC(CCCCCCCC)CCCCCCCC] |
| 2437 | 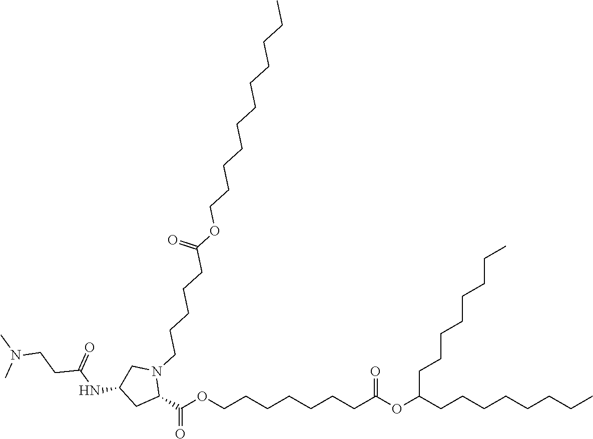 [SMILES: CCCCCCCCCCCOC(=O)CCCCCN1C[C@@H](NC(=O)CCN(C)C)C[C@H]1C(=O)OCCCCCCCC(=O)OC(CCCCCCCC)CCCCCCCC] |
| 2440 | 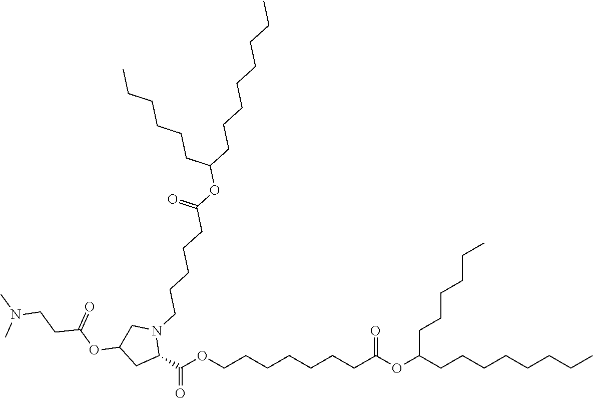 [SMILES: CCCCCCCCC(CCCCCC)OC(=O)CCCCCCCOC(=O)[C@@H]1CC(OC(=O)CCN(C)C)CN1CCCCCC(=O)OC(CCCCCC)CCCCCCCC] |
| 2452 | 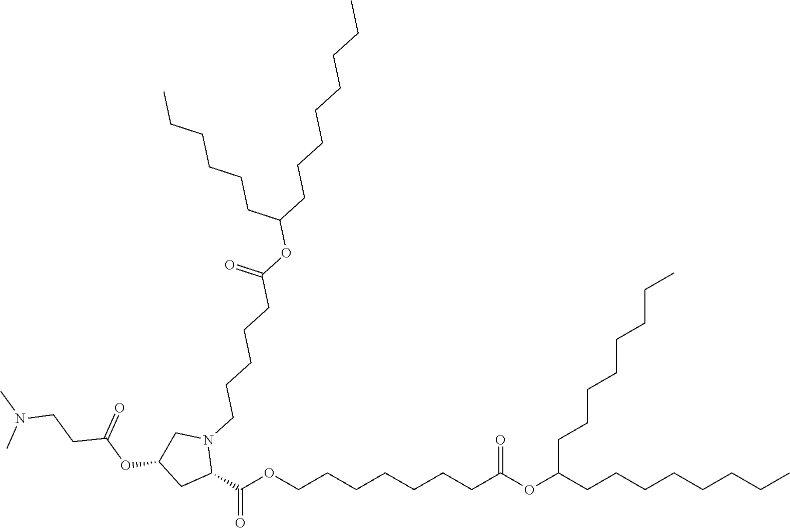 [SMILES: CCCCCCCCC(CCCCCC)OC(=O)CCCCCN1C[C@@H](OC(=O)CCN(C)C)C[C@H]1C(=O)OCCCCCCCC(=O)OC(CCCCCCCC)CCCCCCCC] |
| 2232 | 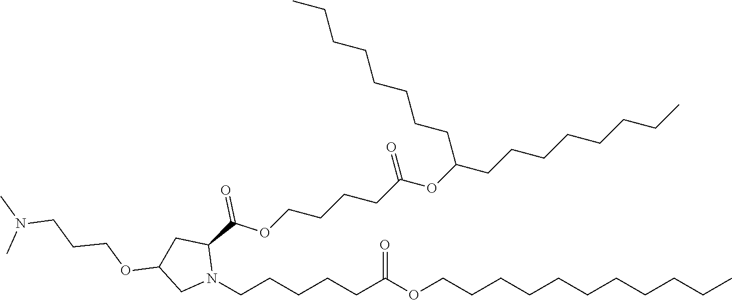 [SMILES: CCCCCCCCCCCOC(=O)CCCCCN1CC(OCCCN(C)C)C[C@H]1C(=O)OCCCCC(=O)OC(CCCCCCCC)CCCCCCCC] |
| 2350 | 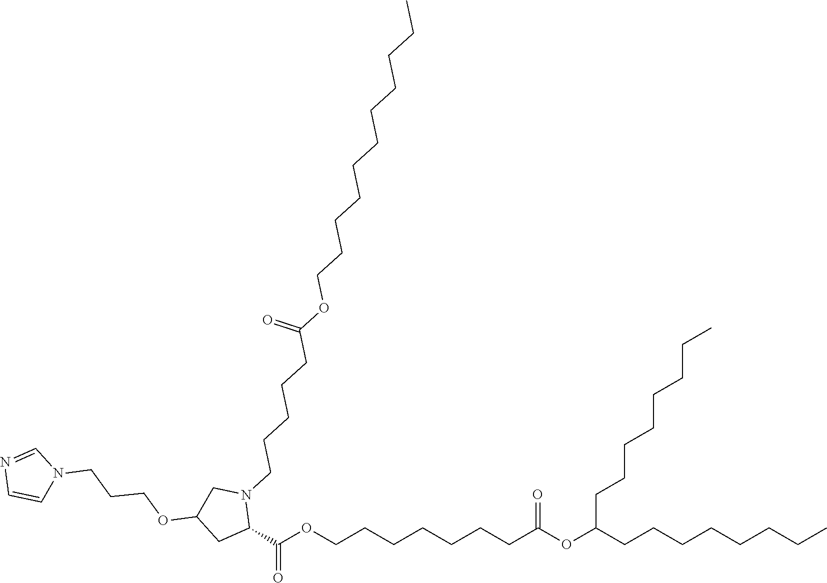 [SMILES: CCCCCCCCCCCOC(=O)CCCCCN1CC(OCCCn2ccnc2)C[C@H]1C(=O)OCCCCCCCC(=O)OC(CCCCCCCC)CCCCCCCC] |
| 2351 | 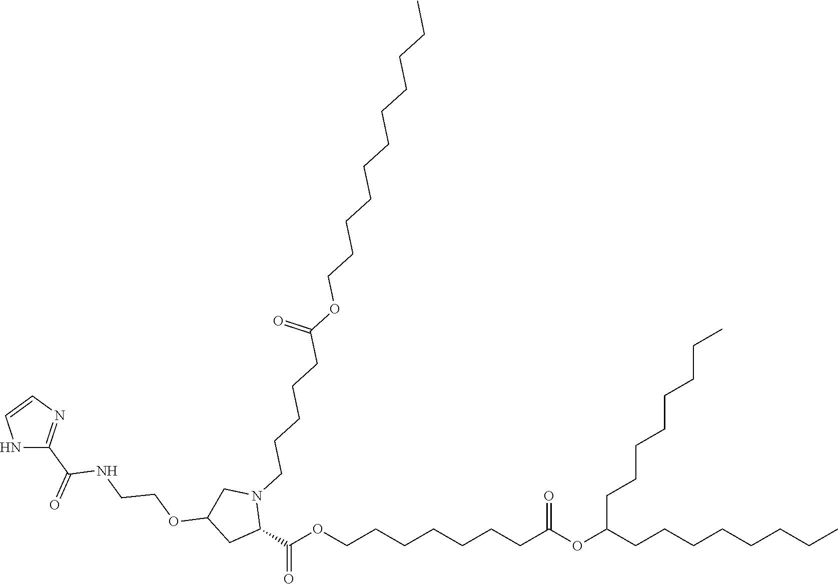 [SMILES: CCCCCCCCCCCOC(=O)CCCCCN1CC(OCCNC(=O)c2ncc[nH]2)C[C@H]1C(=O)OCCCCCCCC(=O)OC(CCCCCCCC)CCCCCCCC] |
| 2359 | 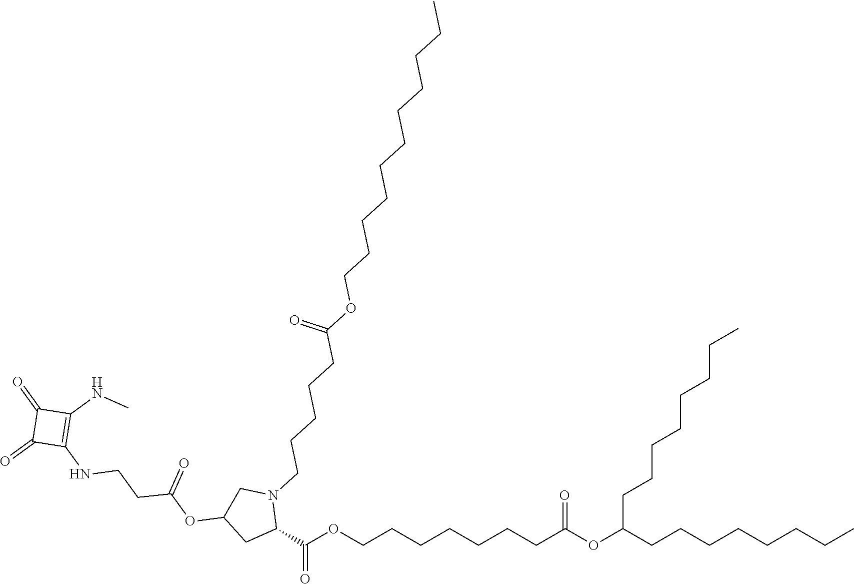 [SMILES: CCCCCCCCCCCOC(=O)CCCCCN1CC(OC(=O)CCNc2c(NC)c(=O)c2=O)C[C@H]1C(=O)OCCCCCCCC(=O)OC(CCCCCCCC)CCCCCCCC] |
| 2360 | 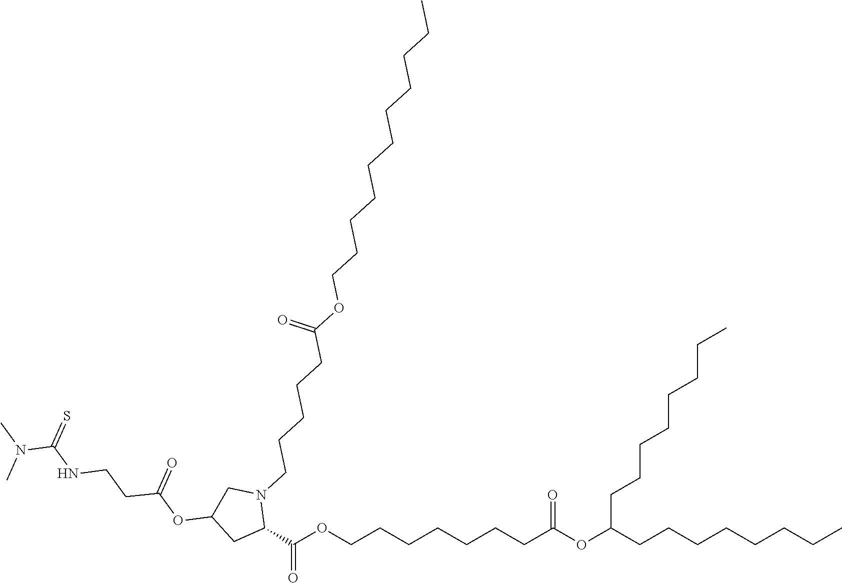 [SMILES: CCCCCCCCCCCOC(=O)CCCCCN1CC(OC(=O)CCNC(=S)N(C)C)C[C@H]1C(=O)OCCCCCCCC(=O)OC(CCCCCCCC)CCCCCCCC] |
| 2363 | 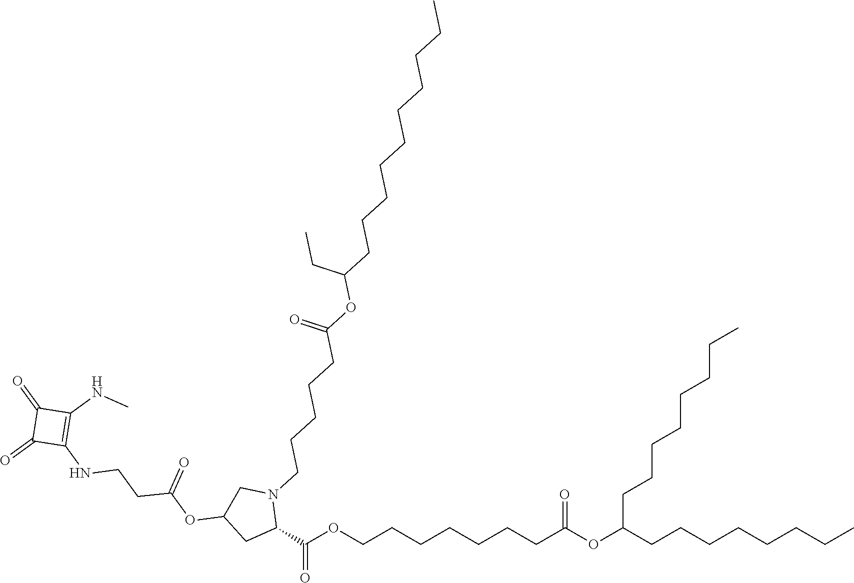 [SMILES: CCCCCCCCCCC(CC)OC(=O)CCCCCN1CC(OC(=O)CCNc2c(NC)c(=O)c2=O)C[C@H]1C(=O)OCCCCCCCC(=O)OC(CCCCCCCC)CCCCCCCC] |
| 2364 | 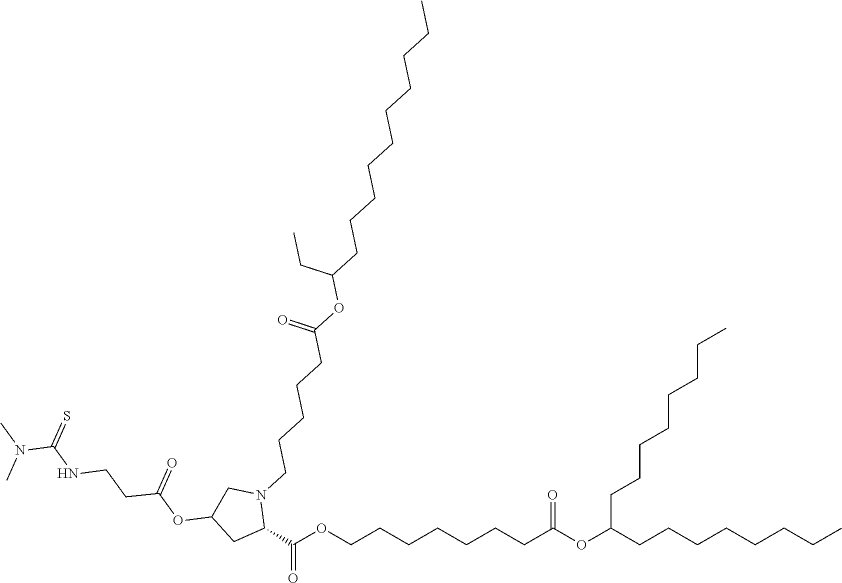 [SMILES: CCCCCCCCCCC(CC)OC(=O)CCCCCN1CC(OC(=O)CCNC(=S)N(C)C)C[C@H]1C(=O)OCCCCCCCC(=O)OC(CCCCCCCC)CCCCCCCC] |
| 2374 | 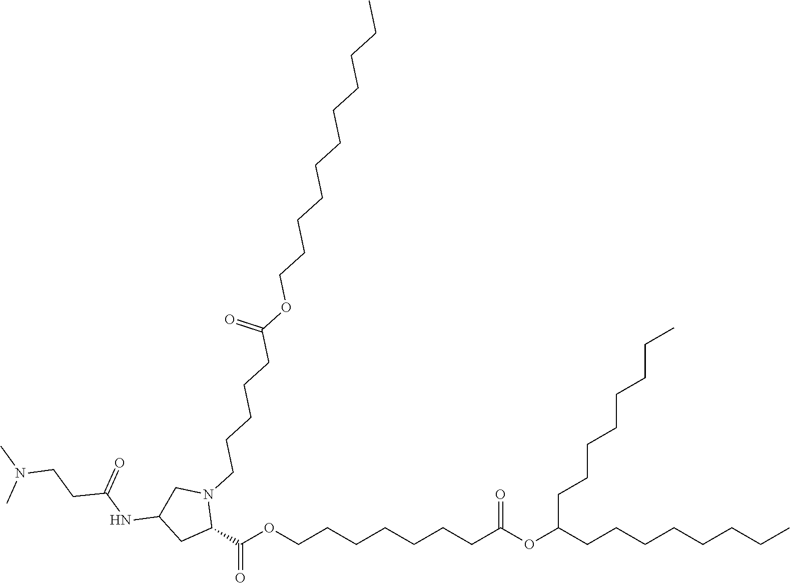 [SMILES: CCCCCCCCCCCOC(=O)CCCCCN1CC(NC(=O)CCN(C)C)C[C@H]1C(=O)OCCCCCCCC(=O)OC(CCCCCCCC)CCCCCCCC] |
| 2422 | 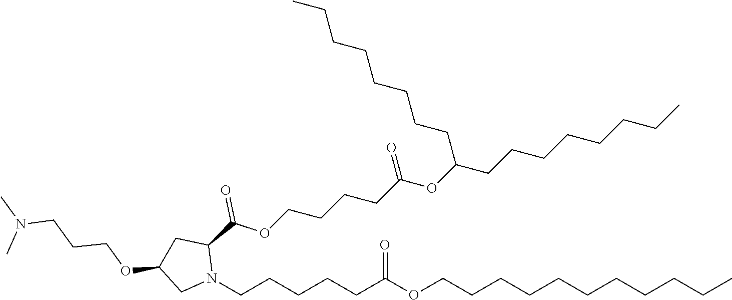 [SMILES: CCCCCCCCCCCOC(=O)CCCCCN1C[C@@H](OCCCN(C)C)C[C@H]1C(=O)OCCCCC(=O)OC(CCCCCCCC)CCCCCCCC] |
| 2423 | 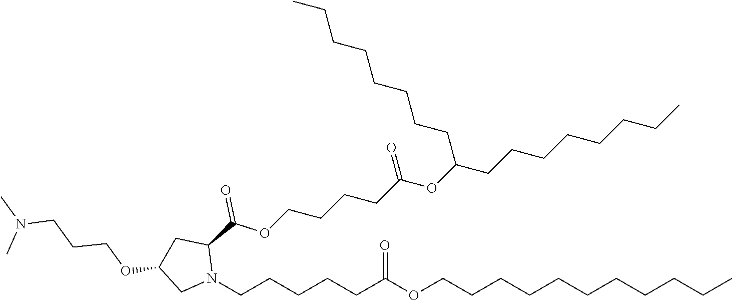 [SMILES: CCCCCCCCCCCOC(=O)CCCCCN1C[C@H](OCCCN(C)C)C[C@H]1C(=O)OCCCCC(=O)OC(CCCCCCCC)CCCCCCCC] |
| 2429 | 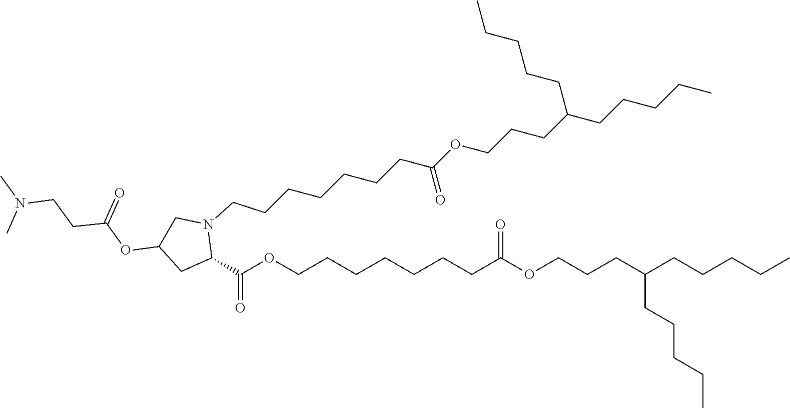 [SMILES: CCCCCC(CCCCC)CCCOC(=O)CCCCCCCOC(=O)[C@@H]1CC(OC(=O)CCN(C)C)CN1CCCCCCCC(=O)OCCCC(CCCCC)CCCCC] |
| 2430 | 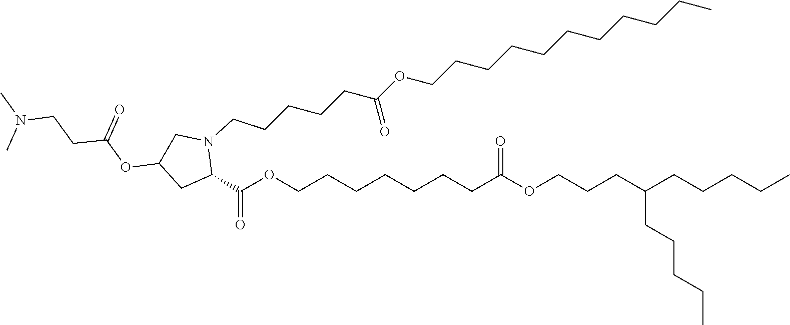 [SMILES: CCCCCCCCCCCOC(=O)CCCCCN1CC(OC(=O)CCN(C)C)C[C@H]1C(=O)OCCCCCCCC(=O)OCCCC(CCCCC)CCCCC] |
| 2431 | 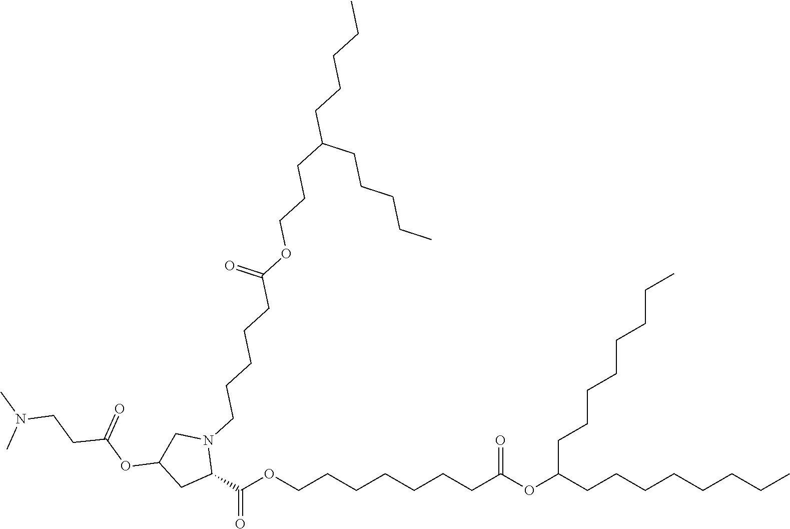 [SMILES: CCCCCCCCC(CCCCCCCC)OC(=O)CCCCCCCOC(=O)[C@@H]1CC(OC(=O)CCN(C)C)CN1CCCCCC(=O)OCCCC(CCCCC)CCCCC] |
| 2438 | 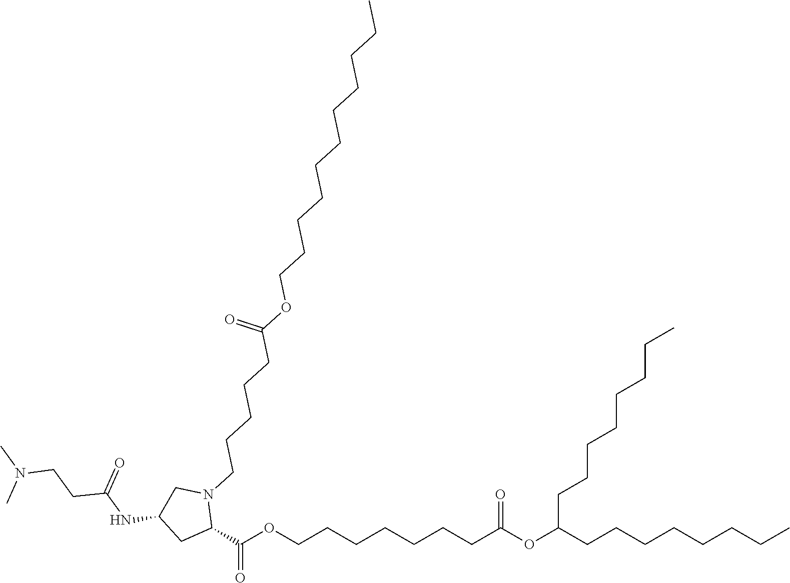 [SMILES: CCCCCCCCCCCOC(=O)CCCCCN1C[C@@H](NC(=O)CCN(C)C)C[C@H]1C(=O)OCCCCCCCC(=O)OC(CCCCCCCC)CCCCCCCC] |
| 2451 | 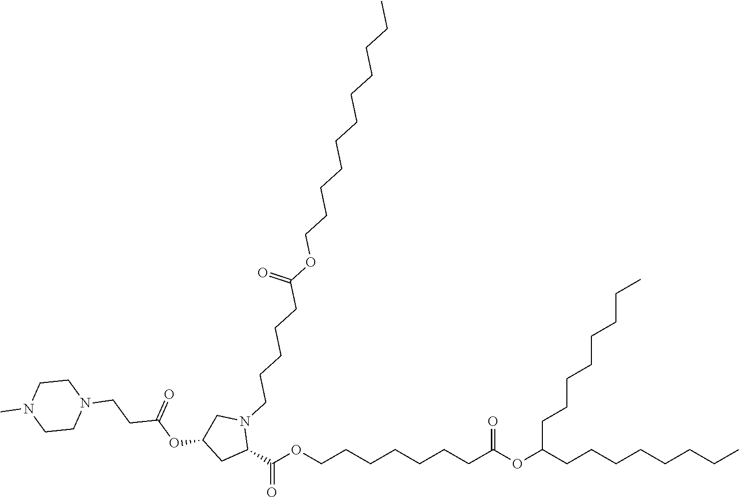 [SMILES: CCCCCCCCCCCOC(=O)CCCCCN1C[C@@H](OC(=O)CCN2CCN(C)CC2)C[C@H]1C(=O)OCCCCCCCC(=O)OC(CCCCCCCC)CCCCCCCC] |
| 2455 | 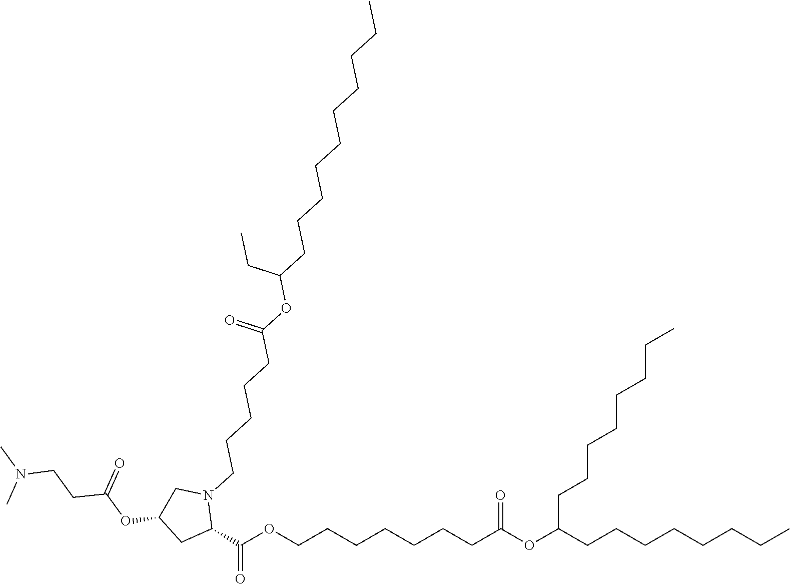 [SMILES: CCCCCCCCCCC(CC)OC(=O)CCCCCN1C[C@@H](OC(=O)CCN(C)C)C[C@H]1C(=O)OCCCCCCCC(=O)OC(CCCCCCCC)CCCCCCCC] |
| 1 | 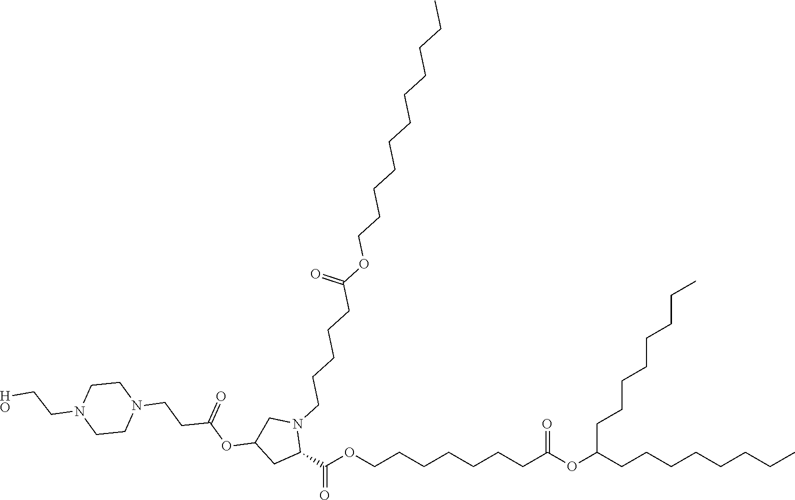 [SMILES: CCCCCCCCCCCOC(=O)CCCCCN1CC(OC(=O)CCN2CCN(CCO)CC2)C[C@H]1C(=O)OCCCCCCCC(=O)OC(CCCCCCCC)CCCCCCCC] |
| 2 | 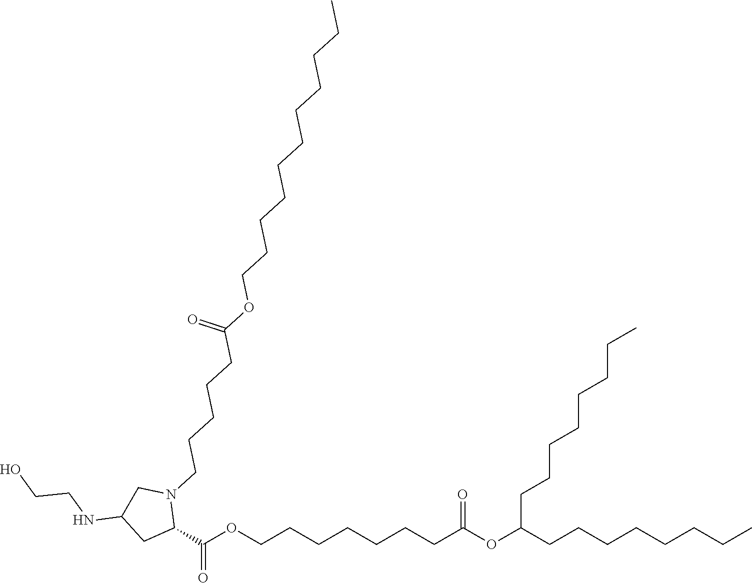 [SMILES: CCCCCCCCCCCOC(=O)CCCCCN1CC(NCCO)C[C@H]1C(=O)OCCCCCCCC(=O)OC(CCCCCCCC)CCCCCCCC] |
| 3 | 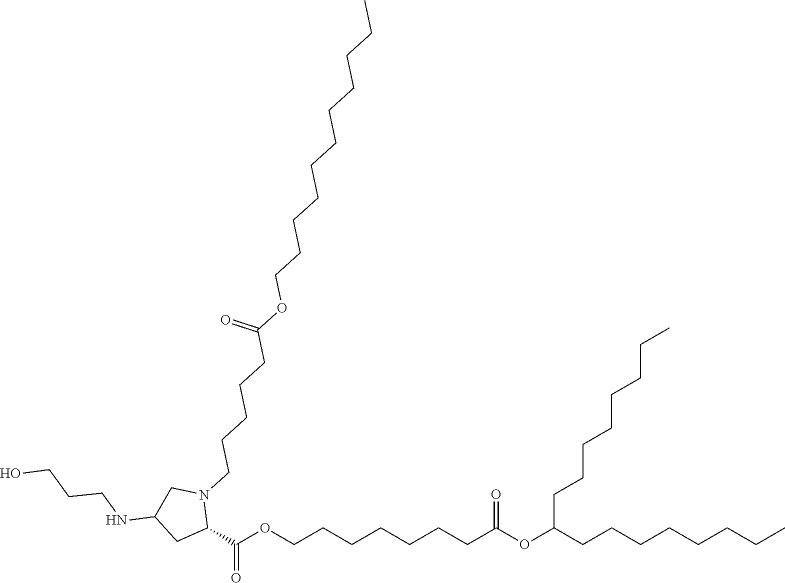 [SMILES: CCCCCCCCCCCOC(=O)CCCCCN1CC(NCCCO)C[C@H]1C(=O)OCCCCCCCC(=O)OC(CCCCCCCC)CCCCCCCC] |
| 4 | 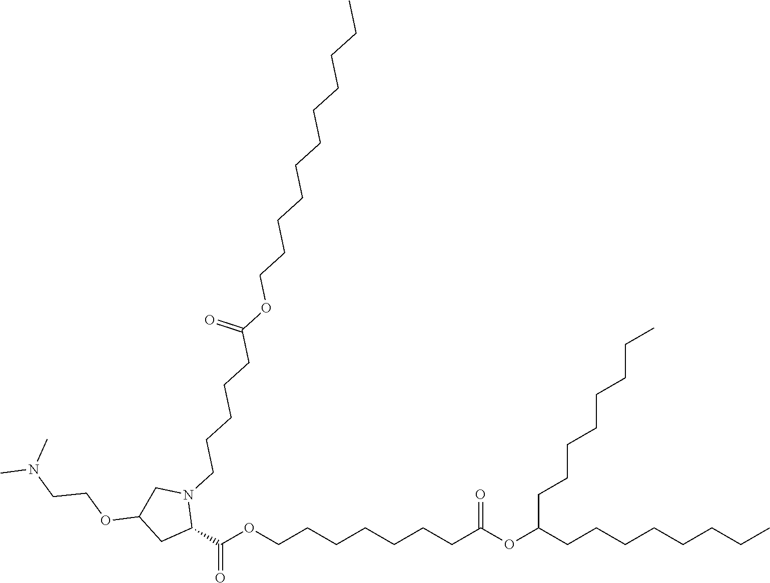 [SMILES: CCCCCCCCCCCOC(=O)CCCCCN1CC(OCCN(C)C)C[C@H]1C(=O)OCCCCCCCC(=O)OC(CCCCCCCC)CCCCCCCC] |
| 5 | 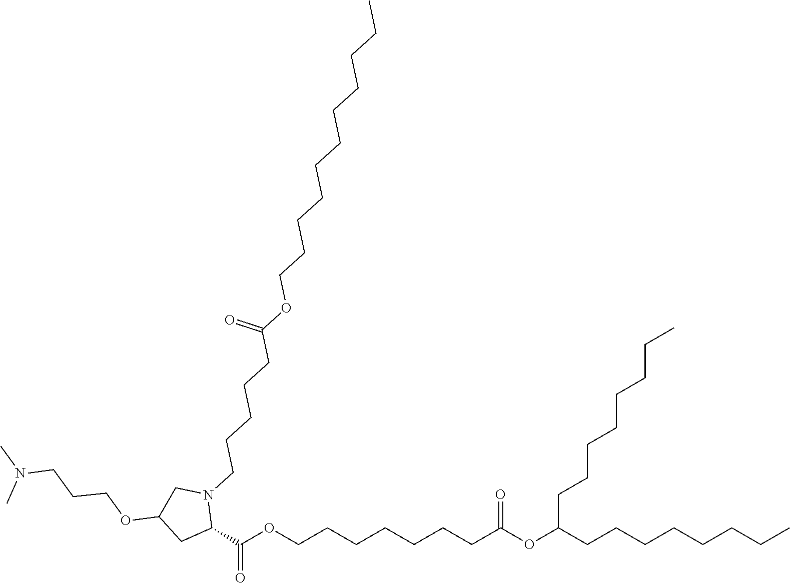 [SMILES: CCCCCCCCCCCOC(=O)CCCCCN1CC(OCCCN(C)C)C[C@H]1C(=O)OCCCCCCCC(=O)OC(CCCCCCCC)CCCCCCCC] |
| 6 | 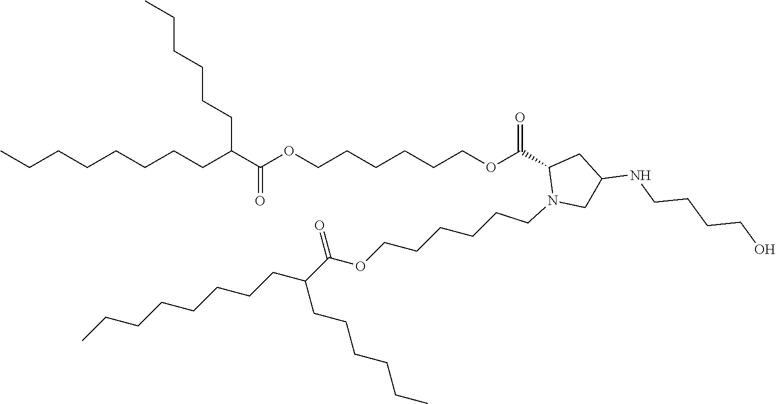 [SMILES: CCCCCCCCC(CCCCCC)C(=O)OCCCCCCOC(=O)[C@@H]1CC(NCCCCO)CN1CCCCCCOC(=O)C(CCCCCC)CCCCCCCC] |
| 7 | 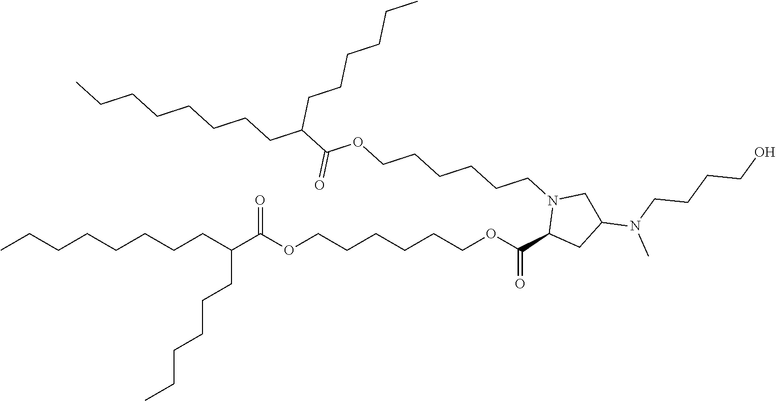 [SMILES: CCCCCCCCC(CCCCCC)C(=O)OCCCCCCOC(=O)[C@@H]1CC(N(C)CCCCO)CN1CCCCCCOC(=O)C(CCCCCC)CCCCCCCC] |
| 8 | 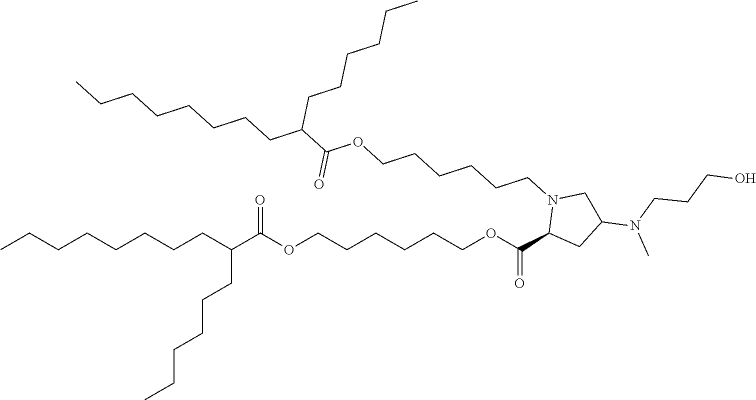 [SMILES: CCCCCCCCC(CCCCCC)C(=O)OCCCCCCOC(=O)[C@@H]1CC(N(C)CCCO)CN1CCCCCCOC(=O)C(CCCCCC)CCCCCCCC] |
| 9 | 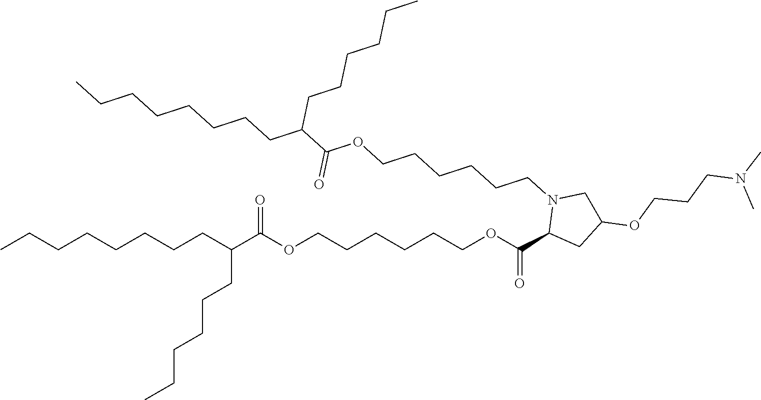 [SMILES: CCCCCCCCC(CCCCCC)C(=O)OCCCCCCOC(=O)[C@@H]1CC(OCCCN(C)C)CN1CCCCCCOC(=O)C(CCCCCC)CCCCCCCC] |
| 10 | 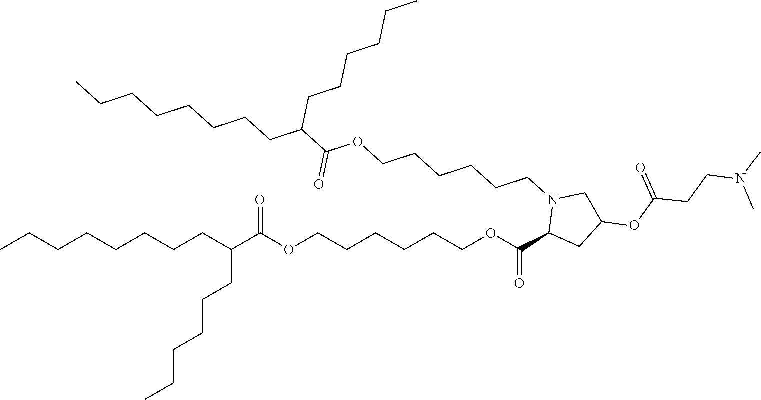 [SMILES: CCCCCCCCC(CCCCCC)C(=O)OCCCCCCOC(=O)[C@@H]1CC(OC(=O)CCN(C)C)CN1CCCCCCOC(=O)C(CCCCCC)CCCCCCCC] |
| 11 | 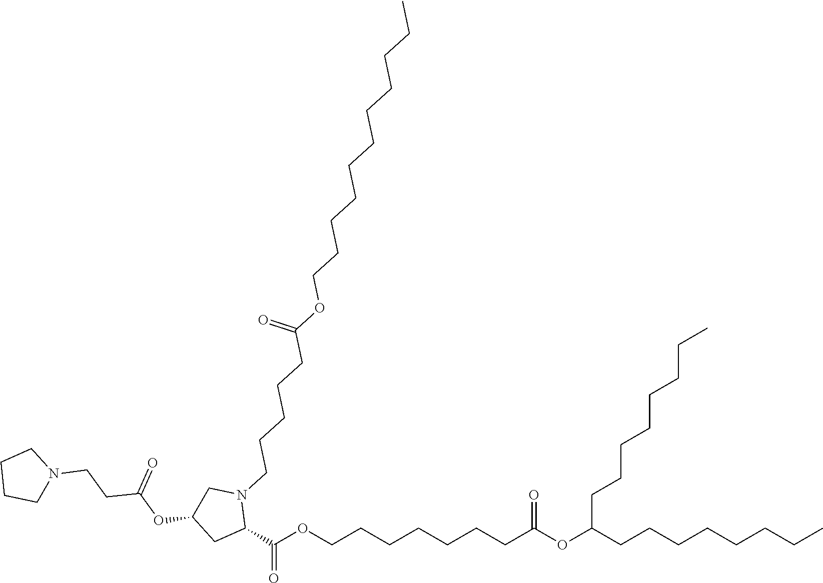 [SMILES: CCCCCCCCCCCOC(=O)CCCCCN1C[C@@H](OC(=O)CCN2CCCC2)C[C@H]1C(=O)OCCCCCCCC(=O)OC(CCCCCCCC)CCCCCCCC] |
| 12. | 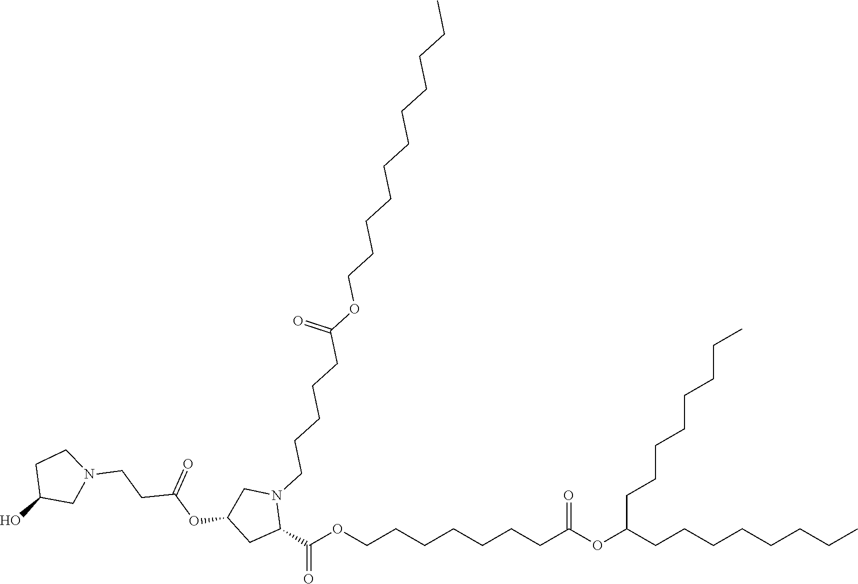 [SMILES: CCCCCCCCCCCOC(=O)CCCCCN1C[C@@H](OC(=O)CCN2CC[C@H](O)C2)C[C@H]1C(=O)OCCCCCCCC(=O)OC(CCCCCCCC)CCCCCCCC] |
| 13. | 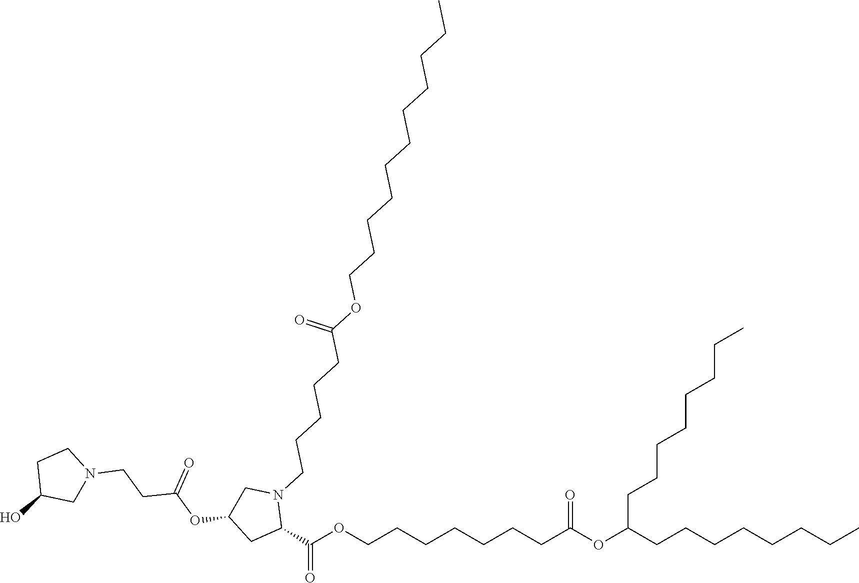 [SMILES: CCCCCCCCCCCOC(=O)CCCCCN1C[C@@H](OC(=O)CCN2CC[C@H](O)C2)C[C@H]1C(=O)OCCCCCCCC(=O)OC(CCCCCCCC)CCCCCCCC] |
| 14. | 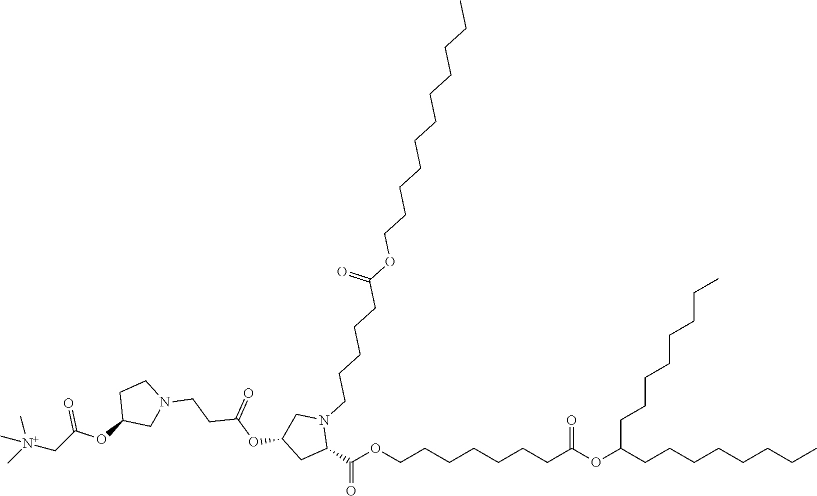 [SMILES: CCCCCCCCCCCOC(=O)CCCCCN1C[C@@H](OC(=O)CCN2CC[C@H](OC(=O)C[N+](C)(C)C)C2)C[C@H]1C(=O)OCCCCCCCC(=O)OC(CCCCCCCC)CCCCCCCC] |
| 15. | 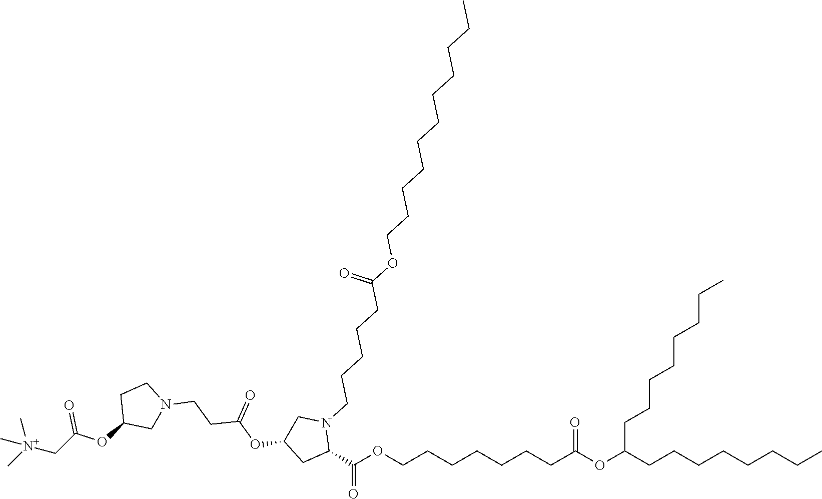 [SMILES: CCCCCCCCCCCOC(=O)CCCCCN1C[C@@H](OC(=O)CCN2CC[C@H](OC(=O)C[N+](C)(C)C)C2)C[C@H]1C(=O)OCCCCCCCC(=O)OC(CCCCCCCC)CCCCCCCC] |
| 16. | 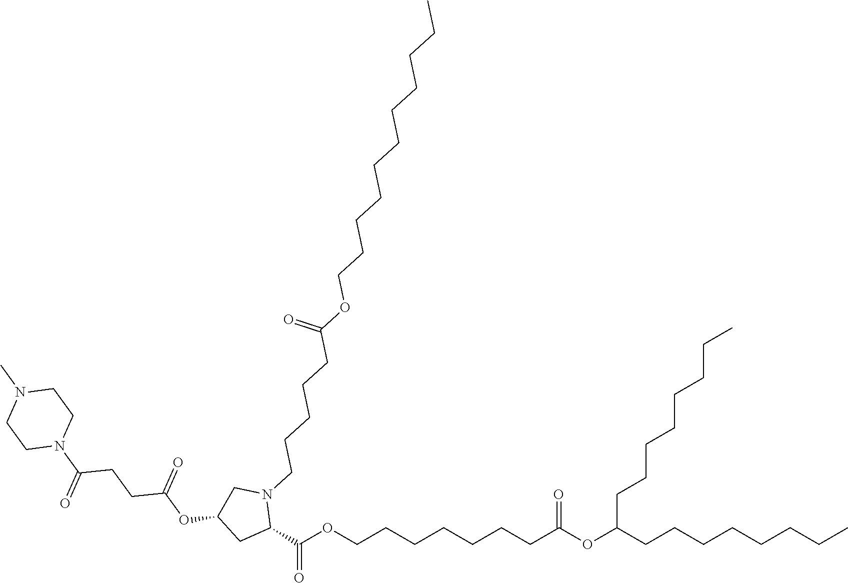 [SMILES: CCCCCCCCCCCOC(=O)CCCCCN1C[C@@H](OC(=O)CCC(=O)N2CCN(C)CC2)C[C@H]1C(=O)OCCCCCCCC(=O)OC(CCCCCCCC)CCCCCCCC] |
| 17. | 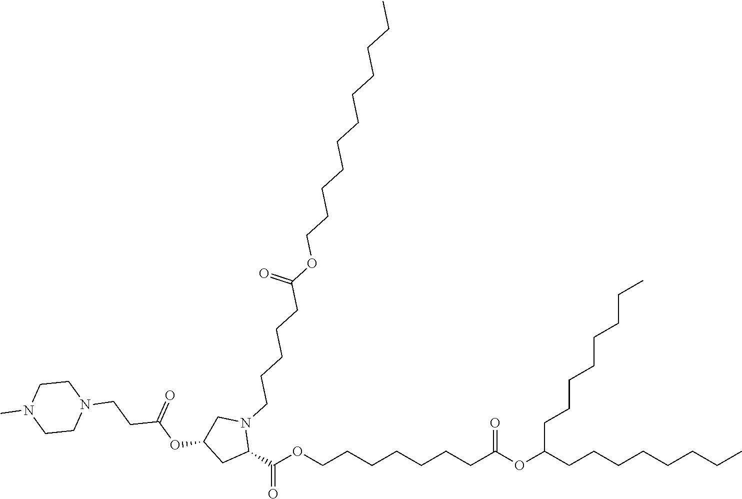 [SMILES: CCCCCCCCCCCOC(=O)CCCCCN1C[C@@H](OC(=O)CCN2CCN(C)CC2)C[C@H]1C(=O)OCCCCCCCC(=O)OC(CCCCCCCC)CCCCCCCC] |
| 18. | 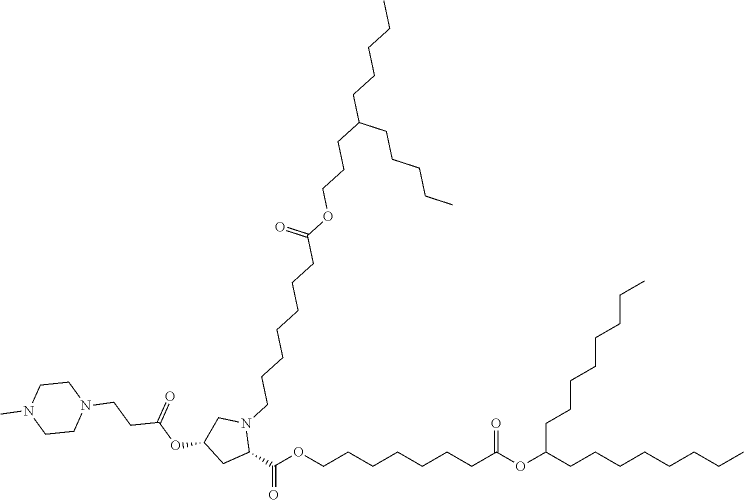 [SMILES: CCCCCCCCC(CCCCCCCC)OC(=O)CCCCCCCOC(=O)[C@@H]1C[C@H](OC(=O)CCN2CCN(C)CC2)CN1CCCCCCCC(=O)OCCCC(CCCCC)CCCCC] |
| 19. | 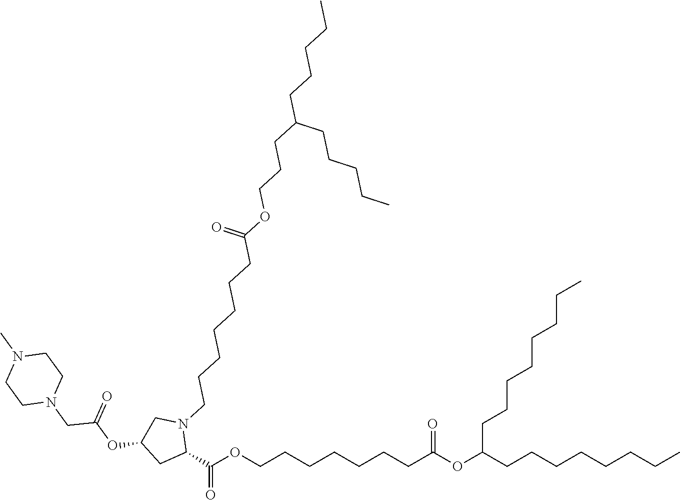 [SMILES: CCCCCCCCC(CCCCCCCC)OC(=O)CCCCCCCOC(=O)[C@@H]1C[C@H](OC(=O)CN2CCN(C)CC2)CN1CCCCCCCC(=O)OCCCC(CCCCC)CCCCC] |
| 20. | 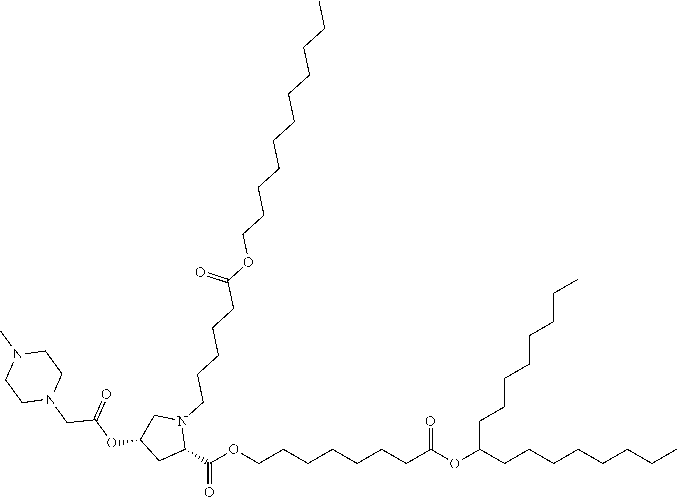 [SMILES: CCCCCCCCCCCOC(=O)CCCCCN1C[C@@H](OC(=O)CN2CCN(C)CC2)C[C@H]1C(=O)OCCCCCCCC(=O)OC(CCCCCCCC)CCCCCCCC] |
| 21. | 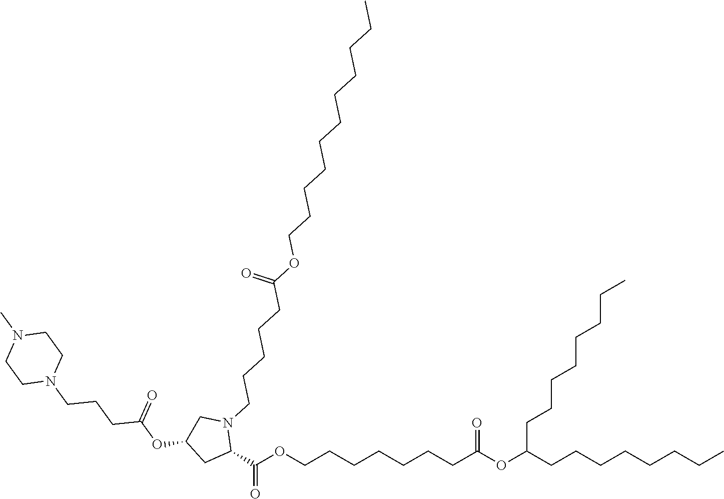 [SMILES: CCCCCCCCCCCOC(=O)CCCCCN1C[C@@H](OC(=O)CCCN2CCN(C)CC2)C[C@H]1C(=O)OCCCCCCCC(=O)OC(CCCCCCCC)CCCCCCCC] |
| 22. | 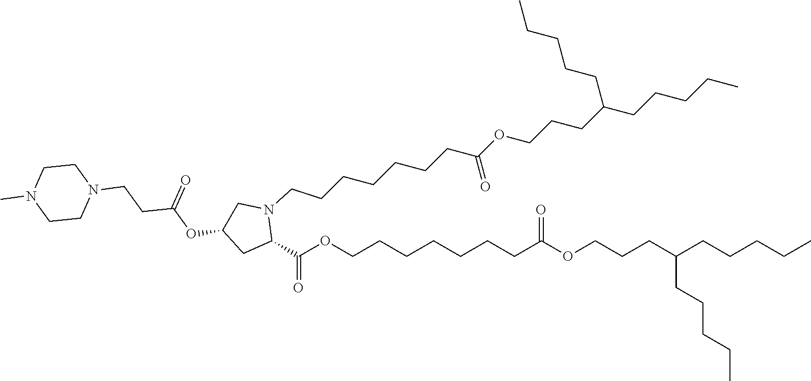 [SMILES: CCCCCC(CCCCC)CCCOC(=O)CCCCCCCOC(=O)[C@@H]1C[C@H](OC(=O)CCN2CCN(C)CC2)CN1CCCCCCCC(=O)OCCCC(CCCCC)CCCCC] |
| 23. | 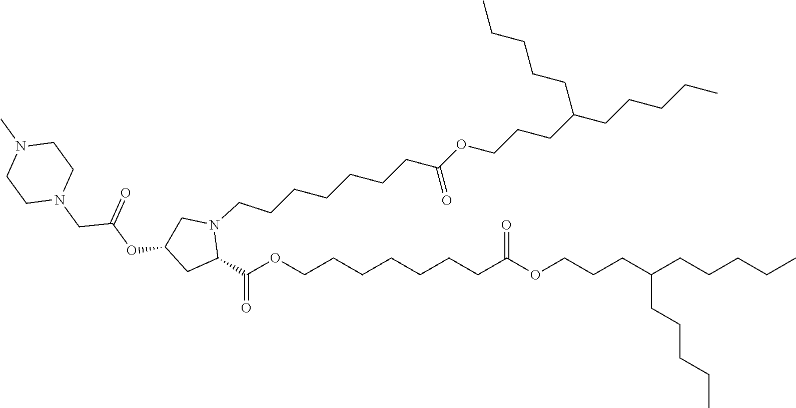 [SMILES: CCCCCC(CCCCC)CCCOC(=O)CCCCCCCOC(=O)[C@@H]1C[C@H](OC(=O)CN2CCN(C)CC2)CN1CCCCCCCC(=O)OCCCC(CCCCC)CCCCC] |
| 24. | 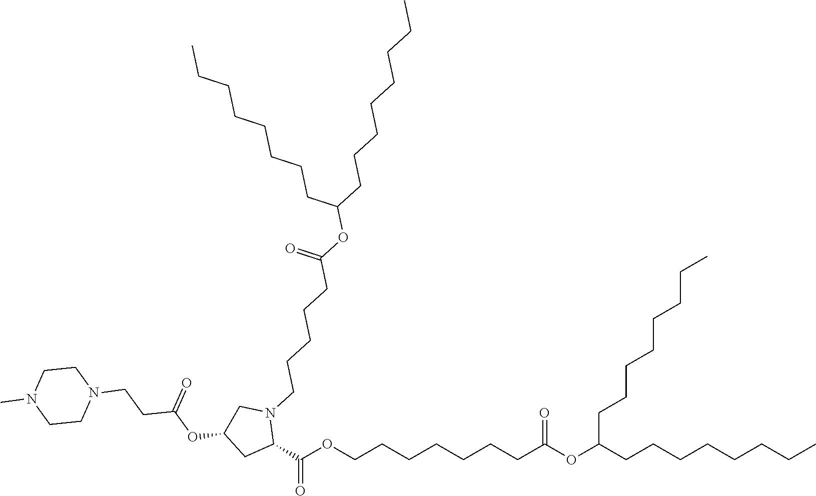 [SMILES: CCCCCCCCC(CCCCCCCC)OC(=O)CCCCCCCOC(=O)[C@@H]1C[C@H](OC(=O)CCN2CCN(C)CC2)CN1CCCCCC(=O)OC(CCCCCCCC)CCCCCCCC] |
| 25. | 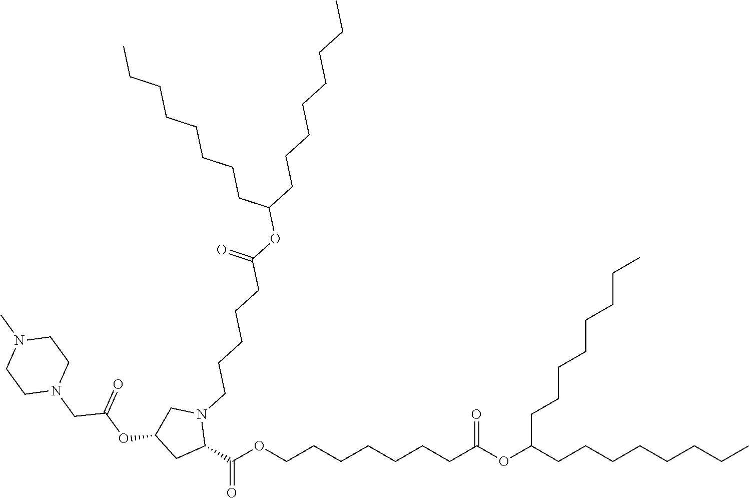 [SMILES: CCCCCCCCC(CCCCCCCC)OC(=O)CCCCCCCOC(=O)[C@@H]1C[C@H](OC(=O)CN2CCN(C)CC2)CN1CCCCCC(=O)OC(CCCCCCCC)CCCCCCCC] |
| 26. | 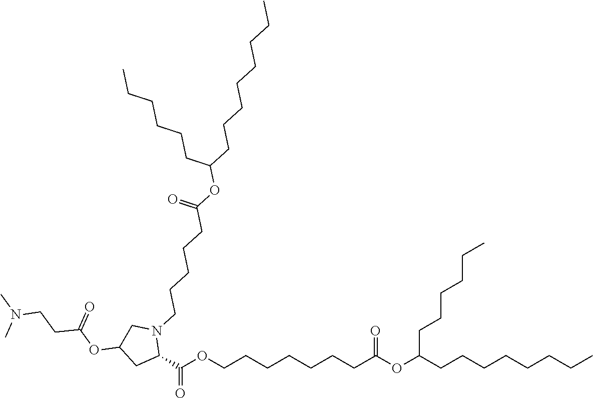 [SMILES: CCCCCCCCC(CCCCCC)OC(=O)CCCCCCCOC(=O)[C@@H]1CC(OC(=O)CCN(C)C)CN1CCCCCC(=O)OC(CCCCCC)CCCCCCCC] |
| 27. | 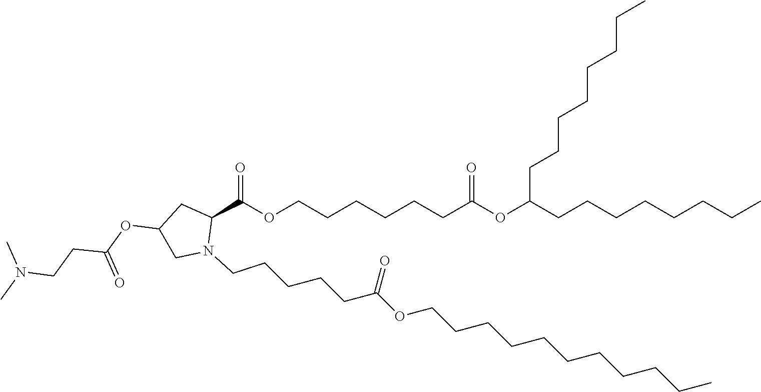 [SMILES: CCCCCCCCCCCOC(=O)CCCCCN1CC(OC(=O)CCN(C)C)C[C@H]1C(=O)OCCCCCCC(=O)OC(CCCCCCCC)CCCCCCCC] |
| 28. | 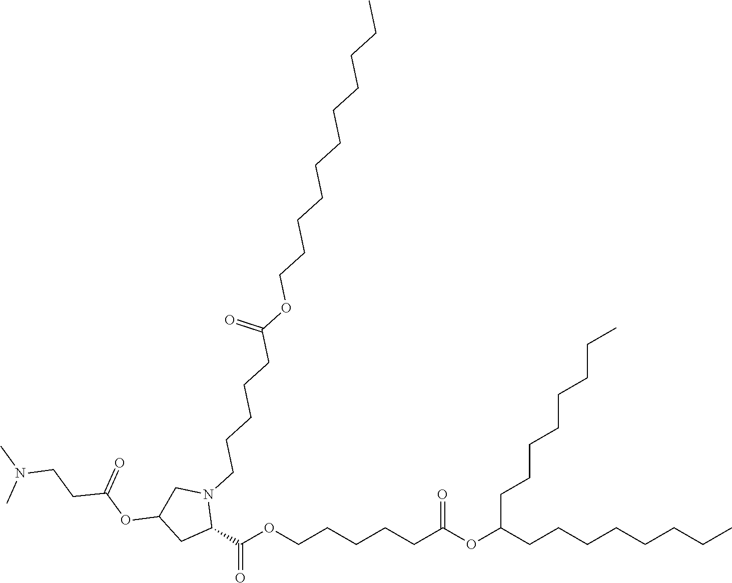 [SMILES: CCCCCCCCCCCOC(=O)CCCCCN1CC(OC(=O)CCN(C)C)C[C@H]1C(=O)OCCCCCC(=O)OC(CCCCCCCC)CCCCCCCC] |
| 29. | 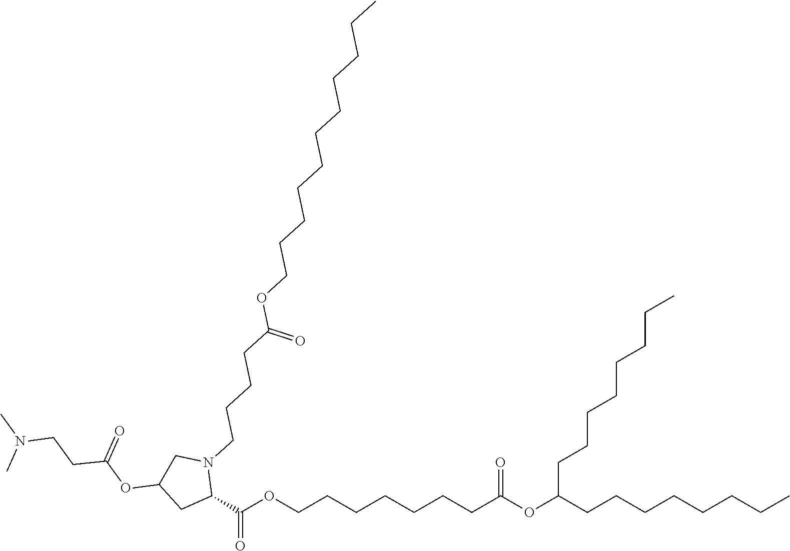 [SMILES: CCCCCCCCCCCOC(=O)CCCCN1CC(OC(=O)CCN(C)C)C[C@H]1C(=O)OCCCCCCCC(=O)OC(CCCCCCCC)CCCCCCCC] |
| 30. | 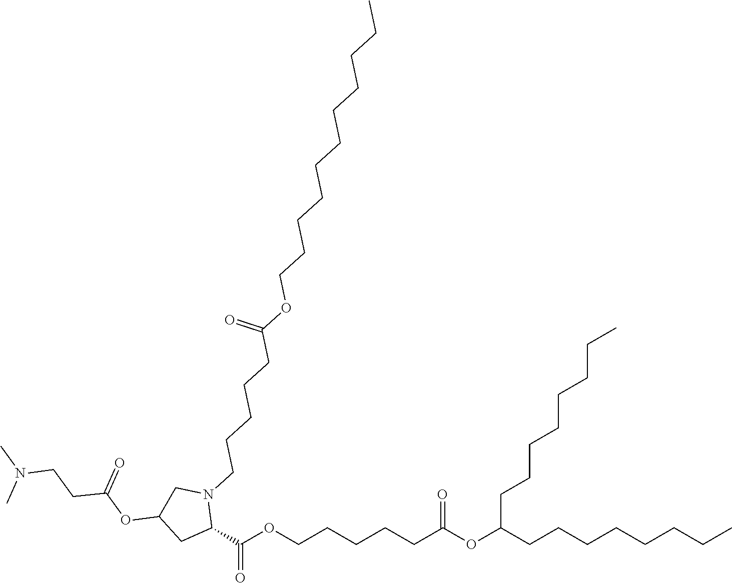 [SMILES: CCCCCCCCCCCOC(=O)CCCCCN1CC(OC(=O)CCN(C)C)C[C@H]1C(=O)OCCCCCC(=O)OC(CCCCCCCC)CCCCCCCC] |
| 31. | 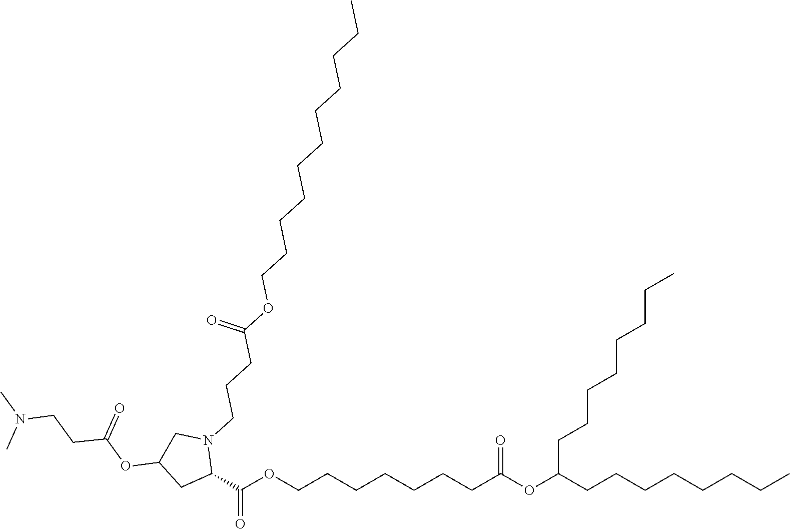 [SMILES: CCCCCCCCCCCOC(=O)CCCN1CC(OC(=O)CCN(C)C)C[C@H]1C(=O)OCCCCCCCC(=O)OC(CCCCCCCC)CCCCCCCC] |
| 32. | 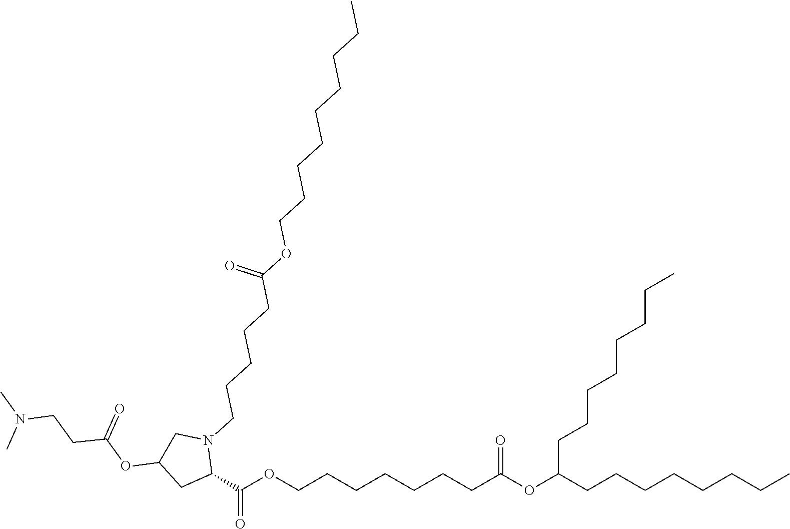 [SMILES: CCCCCCCCCOC(=O)CCCCCN1CC(OC(=O)CCN(C)C)C[C@H]1C(=O)OCCCCCCCC(=O)OC(CCCCCCCC)CCCCCCCC] |
| 33. | 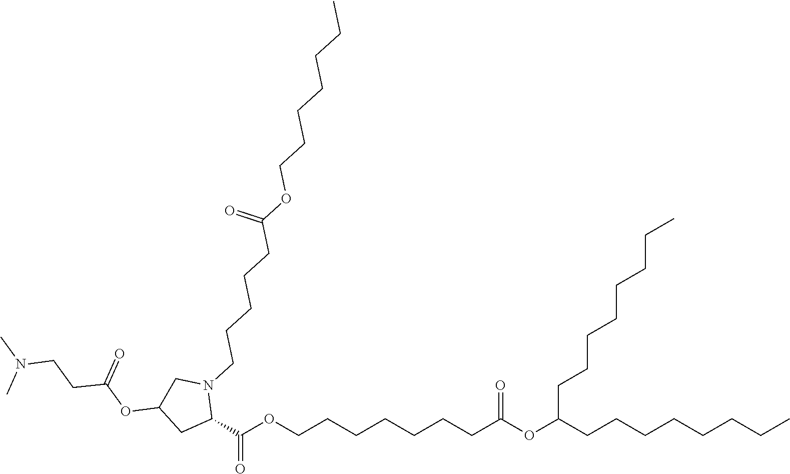 [SMILES: CCCCCCCCC(CCCCCCCC)OC(=O)CCCCCCCOC(=O)[C@@H]1CC(OC(=O)CCN(C)C)CN1CCCCCC(=O)OCCCCCCC] |
| 2310 | heptadecan-9-yl 8-[(2S,4R)-4-{[3-(dimethylamino)propanoyl]oxy}-1-[6-oxo-6-(undecyloxy)hexyl]piperidine-2-carbonyloxy]octanoate |
| 2309 | heptadecan-9-yl 8-[(2S,4S)-4-{[3-(dimethylamino)propanoyl]oxy}-1-[6-oxo-6-(undecyloxy)hexyl]piperidine-2-carbonyloxy]octanoate |
| 2308 | heptadecan-9-yl 8-[(2S,5S)-5-{[3-(dimethylamino)propanoyl]oxy}-1-[6-oxo-6-(undecyloxy)hexyl]piperidine-2-carbonyloxy]octanoate |
| 2307 | heptadecan-9-yl 8-[(2S,5R)-5-{[3-(dimethylamino)propanoyl]oxy}-1-[6-oxo-6-(undecyloxy)hexyl]piperidine-2-carbonyloxy]octanoate |
| 2306 | heptadecan-9-yl 8-[(2S,4S)-4-{[3-(dimethylamino)propanoyl]oxy}-1-[6-oxo-6-(undecyloxy)hexyl]pyrrolidine-2-carbonyloxy]octanoate |
| 2305 | heptadecan-9-yl 8-[(2S,4R)-4-{[3-(dimethylamino)propanoyl]oxy}-1-[6-oxo-6-(undecyloxy)hexyl]pyrrolidine-2-carbonyloxy]octanoate |
| 2304 | heptadecan-9-yl 8-[(2S)-4-{[3-(dimethylamino)propanoyl]oxy}-1-[6-oxo-6-(undecyloxy)hexanoyl]pyrrolidine-2-carbonyloxy]octanoate |
| 2298 | 5-[(2S)-4-{[3-(dimethylamino)propanoyl]oxy}-2-[({7-[(2-octyldecanoyl)oxy]heptyl}oxy)carbonyl]pyrrolidin-1-yl]pentyl dodecanoate |
| 2297 | heptadecan-9-yl 8-[(2S)-4-[(4-hydroxybutanoyl)oxy]-1-[6-oxo-6-(undecyloxy)hexyl]pyrrolidine-2-carbonyloxy]octanoate |
| 2296 | 5-[(2S)-4-{[3-(dimethylamino)propanoyl]oxy}-2-({[8-(heptadecan-9-yloxy)-8-oxooctyl]oxy}carbonyl)pyrrolidin-1-yl]pentyl dodecanoate |
| 2295 | heptadecan-9-yl 9-[(2S)-4-{[3-(dimethylamino)propanoyl]oxy}-1-[6-oxo-6-(undecyloxy)hexyl]pyrrolidine-2-carbonyloxy]nonanoate |
| 2294 | heptadecan-9-yl 7-[(2S)-4-{[3-(dimethylamino)propanoyl]oxy}-1-[6-oxo-6-(undecyloxy)hexyl]pyrrolidine-2-carbonyloxy]heptanoate |
| 2293 | (5S)-5-{[2-(dimethylamino)ethoxy]carbonyl}-1-[6-oxo-6-(undecyloxy)hexyl]pyrrolidin-3-yl 1-heptadecan-9-yl octanoate |
| 2292 | heptadecan-9-yl 8-[(2S)-4-{[4-(dimethylamino)butanoyl]oxy}-1-[6-oxo-6-(undecyloxy)hexyl]pyrrolidine-2-carbonyloxy]octanoate |
| 2291 | heptadecan-9-yl 8-[(2S)-4-({3-[(2-hydroxyethyl)(methyl)amino]propanoyl}oxy)-1-[6-oxo-6-(undecyloxy)hexyl]pyrrolidine-2-carbonyloxy] |
| | octanoate |
| 2290 | heptadecan-9-yl 8-[(2S)-1-[6-oxo-6-(undecyloxy)hexyl}-4-{[3-(pyrrolidin-1-yl)propanoyl]oxy}pyrrolidine-2-carbonyloxy]octanoate |
| 2270 | heptadecan-9-yl 8-[(2S)-4-hydroxy-1-[6-oxo-6-(undecyloxy)hexyl]pyrrolidine-2-carbonyloxy]octanoate |
| 2260 | undecyl 6-[(2S)-2-({[5-(heptadecan-9-yloxy)-5-oxopentyl]oxy}carbonyl)-4-hydroxypyrrolidin-1-yl]hexanoate |
| 2240 | heptadecan-9-yl 5-{[1-(2-hydroxyethyl)-4-[3-oxo-3-(undecyloxy)propoxy]pyrrolidin-3-yl]oxy}pentanoate |
| 2231 | heptadecan-9-yl 8-[(2S)-4-{[3-(dimethylamino)propanoyl]oxy}-1-[6-oxo-6-(undecyloxy)hexyl]pyrrolidine-2-carbonyloxy]octanoate |
| 2230 | undecyl 6-[(2S)-4-{[3-(dimethylamino)propanoyl]oxy}-2-({[5-(heptadecan-9-yloxy)-5-oxopentyl]oxy}carbonyl)pyrrolidin-1-yl]hexanoate |
| 2329 | 8-(heptadecan-9-yloxy)-8-oxooctyl (2S)-4-((3-(dimethylamino)butanoyl)oxy)-1-(6-oxo-6-(undecyloxy)hexyl)pyrrolidine-2-carboxylate |
| 2336 | 8-(heptadecan-9-yloxy)-8-oxooctyl (2S)-1-(6-oxo-6-(undecyloxy)hexyl)-4-(2-(pyrrolidin-1-yl)acetoxy)pyrrolidine-2-carboxylate |
| 2337 | 8-(heptadecan-9-yloxy)-8-oxooctyl (2S)-4-((3-(dimethylamino)propanoyl)oxy)-1-(7-oxo-7-(undecyloxy)heptyl)pyrrolidine-2-carboxylate |
| 2338 | 8-(heptadecan-9-yloxy)-8-oxooctyl (2S)-4-((3-(dimethylamino)propanoyl)oxy)-1-(5-oxo-5-(undecyloxy)pentyl)pyrrolidine-2-carboxylate |
| 2339 | 8-(heptadecan-9-yloxy)-8-oxooctyl (2S)-4-((3-(1H-imidazol-1-yl)propanoyl)oxy)-1-(6-oxo-6-(undecyloxy)hexyl)pyrrolidine-2-carboxylate |
| 2340 | 8-oxo-8-(pentadecan-7-yloxy)octyl (2S)-4-((3-(dimethylamino)propanoyl)oxy)-1-(6-oxo-6-(undecyloxy)hexyl)pyrrolidine-2-carboxylate |
| 2341 | 8-oxo-8-(tridecan-7-yloxy)octyl (2S)-4-((3-(dimethylamino)propanoyl)oxy)-1-(6-oxo-6-(undecyloxy)hexyl)pyrrolidine-2-carboxylate |
| 2342 | 8-(heptadecan-9-yloxy)-8-oxooctyl (2S)-4-((3-(dimethylamino)propanoyl)oxy)-1-(6-((9-methyldecyl)oxy)-6-oxohexyl)pyrrolidine-2- |
| | carboxylate |
| 2343 | 8-(heptadecan-9-yloxy)-8-oxooctyl (2S)-4-((3-(dimethylamino)propanoyl)oxy)-1-(6-(heptadecan-9-yloxy)-6-oxohexyl)pyrrolidine-2- |
| | carboxylate |
| 2344 | 8-(heptadecan-9-yloxy)-8-oxooctyl (2S)-4-((3-(dimethylamino)propanoyl)oxy)-1-(6-oxo-6-(pentadecan-7-yloxy)hexyl)pyrrolidine-2-carboxylate |
| 2345 | 8-(heptadecan-9-yloxy)-8-oxooctyl (2S)-4-((3-(dimethylamino)propanoyl)oxy)-1-(6-oxo-6-(tridecan-7-yloxy)hexyl)pyrrolidine-2-carboxylate |
| 2348 | 8-(heptadecan-9-yloxy)-8-oxooctyl (2S)-4-((3-(2-methyl-1H-imidazol-1-yl)propanoyl)oxy)-1-(6-oxo-6-(undecyloxy)hexyl)pyrrolidine-2- |
| | carboxylate |
| 2349 | 8-(heptadecan-9-yloxy)-8-oxooctyl (2S)-4-((4-(1H-imidazol-1-yl)butanoyl)oxy)-1-(6-oxo-6-(undecyloxy)hexyl)pyrrolidine-2-carboxylate |
| 2352 | 8-(heptadecan-9-yloxy)-8-oxooctyl (2S)-4-((3-(1H-imidazole-2-carboxamido)propanoyl)oxy)-1-(6-oxo-6-(undexyloxy)hexyl)pyrrolidine-2- |
| | carboxylate |
| 2371 | 5-(heptadecan-9-yloxy)-5-oxopentyl (2S,4S)-4-((3-(dimethylamino)propanoyl)oxy)-1-(6-oxo-6-(undecyloxy)hexyl)pyrrolidine-2-carboxylate |
| 2372 | 5-(heptadecan-9-yloxy)-5-oxopentyl (2S,4R)-4-((3-(dimethylamino)propanoyl)oxy)-1-(6-oxo-6-(undecyloxy)hexyl)pyrrolidine-2-carboxylate |
| 2373 | 8-(heptadecan-9-yloxy)-8-oxooctyl (2S)-4-(3-(dimethylamino)propanamido)-1-(6-oxo-6-(undecyloxy)hexyl)pyrrolidine-1-carboxylate |
| 2375 | 8-(heptadecan-9-yloxy)-8-oxooctyl (2S)-4-(4-((dimethylamino)methyl)-1H-1,2,3-triazol-1-yl)-1-(6-oxo-6-(undecyloxy)hexyl)pyrrolidine-2- |
| | carboxylate |
| 2376 | 5-(heptadecan-9-yloxy)-5-oxopentyl (2S)-1-(6-oxo-6-(undecyloxy)hexyl)-4-(3-(pyrrolidin-1-yl)propoxy)pyrrolidine-2-carboxylate |
| 2377 | 8-(heptadecan-9-yloxy)-8-oxooctyl (2S)-4-(3-(dimethylamino)propoxy)-1-(6-oxo-6-(undecyloxy)hexyl)pyrrolidine-2-carboxylate |
| 2436 | 8-(heptadecan-9-yloxy)-8-oxooctyl (2S,4S)-4-(4-((dimethylamino)methyl)-1H-1,2,3-triazol-1-yl)-1-(6-oxo-6-(undecyloxy)hexyl)pyrrolidine- |
| | 2-carboxylate |
| 2436 | 8-(heptadecan-9-yloxy)-8-oxooctyl (2S,4S)-4-(3-(dimethylamino)propanamido)-1-(6-oxo-6-(undecyloxy)hexyl)pyrrolidine-2-carboxylate |
| 2440 | 8-oxo-8-(pentadecan-7-yloxy)octyl (2S)-4-((3-(dimethylamino)propanoyl)oxy)-1-(6-oxo-6-(pentadecan-7-yloxy)hexyl)pyrrolidine-2-carboxylate |
| 2452 | 8-(heptadecan-9-yloxy)-8-oxooctyl (2S,4S)-4-((3-(dimethylamino)propanoyl)oxy)-1-(6-oxo-6-(pentadecan-7-yloxy)hexyl)pyrrolidine-2- |
| | carboxylate |
| 2232 | undecyl 6-[(2S)-4-[3-(dimethylamino)propoxy]-2-({[5-(heptadecan-9-yloxy)-5-oxopentyl]oxy}carbonyl)pyrrolidin-1-yl]hexanoate |
| 2350 | 8-(heptadecan-9-yloxy)-8-oxooctyl (2S)-4-(3-(1H-imidazol-1-yl)propoxy)-1-(6-oxo-6-(undecyloxy)hexyl)pyrrolidine-2-carboxylate |
| 2351 | 8-(heptadecan-9-yloxy)-8-oxooctyl (2S)-4-(2-(1H-imidazole-2-carboxamido)ethoxy)-1-(6-oxo-6-(undecyloxy)hexyl)pyrrolidine-2-carboxylate |
| 2359 | 8-(heptadecan-9-yloxy)-8-oxooctyl (2S)-4-((3-((2-(methylamino)-3,4-dioxocyclobut-1-en-1-yl)amino)propanoyl)oxy)-1-(6-oxo-6-(undecyloxy) |
| | hexyl)pyrrolidine-2-carboxylate |
| 2360 | 8-(heptadecan-9-yloxy)-8-oxooctyl (2S)-4-((3-(3,3-dimethylthioureido)propanoyl)oxy)-1-(6-oxo-6-(undecyloxy)hexyl)pyrrolidine-2-carboxylate |
| 2363 | 8-(heptadecan-9-yloxy)-8-oxooctyl (2S)-4-((3-((2-(methylamino)-3,4-dioxocyclobut-1-en-1-yl)amino)propanoyl)oxy)-1-(6-oxo-6-(tridecan-3- |
| | yloxy)hexyl)pyrrolidine-2-carboxylate |
| 2364 | 8-(heptadecan-9-yloxy)-8-oxooctyl (2S)-4-((3-(3,3-dimethylthioureido)propanoyl)oxy)-1-(6-oxo-6-(tridecan-3-yloxy)hexyl)pyrrolidine- |
| | 2-carboxylate |
| 2374 | 8-(heptadecan-9-yloxy)-8-oxooctyl (2S)-4-((3-(dimethylamino)propyl)amino)-1-(6-oxo-6-(undecyloxy)hexyl)pyrrolidine-2-carboxylate |
| 2422 | 5-(heptadecan-9-yloxy)-5-oxopentyl (2S,4S)-4-(3-(dimethylamino)propoxy)-1-(6-oxo-6-(undecyloxy)hexyl)pyrrolidine-2-carboxylate |
| 2423 | 5-(heptadecan-9-yloxy)-5-oxopentyl (2S,4R)-4-(3-(dimethylamino)propoxy)-1-(6-oxo-6-(undecyloxy)hexyl)pyrrolidine-2-carboxylate |
| 2429 | 8-oxo-8-((4-pentylnonyl)oxy)octyl (2S)-4-((3-(dimethylamino)propanoyl)oxy)-1-(8-oxo-8-((4-pentylnonyl)oxy)octyl)pyrrolidine-2-carboxylate |
| 2430 | 8-oxo-8-((4-pentylnonyl)oxy)octyl (2S)-4-((3-(dimethylamino)propanoyl)oxy)-1-(6-oxo-6-(undecyloxy)hexyl)pyrrolidine-2-carboxylate |
| 2431 | 8-(heptadecan-9-yloxy)-8-oxooctyl (2S)-4-((3-(dimethylamino)propanoyl)oxy)-1-(6-oxo-6-((4-pentylnonyl)oxy)hexyl)pyrrolidine-2-carboxylate |
| 2438 | 8-(heptadecan-9-yloxy)-8-oxooctyl (2S,4S)-4-((3-(dimethylamino)propyl)amino)-1-(6-oxo-6-(undecyloxy)hexyl)pyrrolidine-2-carboxylate |
| 2451 | 8-(heptadecan-9-yloxy)-8-oxooctyl (2S,4S)-4-((3-(4-methylpiperazin-1-yl)propanoyl)oxy)-1-(6-oxo-6-(undecyloxy)hexyl)pyrrolidine-2- |
| | carboxylate |
| 2455 | 8-(heptadecan-9-yloxy)-8-oxooctyl (2S,4S)-4-((3-(dimethylamino)propanoyl)oxy)-1-(6-oxo-6-(tridecan-3-yloxy)hexyl)pyrrolidine-2-carboxylate |
| 1 | 8-(heptadecan-9-yloxy)-8-oxooctyl (2S)-4-((3-(4-(2-hydroxyethyl)piperazin-1-yl)propanoyl)oxy)-1-(6-oxo-6-(undecyloxy)hexyl) |
| | pyrrolidine-2-carboxylate |
| 2 | 8-(heptadecan-9-yloxy)-8-oxooctyl (2S)-4-((2-hydroxyethyl)amino)-1-(6-oxo-6-(undecyloxy)hexyl)pyrrolidine-2-carboxylate |
| 3 | 8-(heptadecan-9-yloxy)-8-oxooctyl (2S)-4-((3-hydroxypropyl)amino)-1-(6-oxo-6-(undecyloxy)hexyl)pyrrolidine-2-carboxylate |
| 4 | 8-(heptadecan-9-yloxy)-8-oxooctyl (2S)-4-(2-(dimethylamino)ethoxy)-1-(6-oxo-6-(undecyloxy)hexyl)pyrrolidine-2-carboxylate |
| 5 | 8-(heptadecan-9-yloxy)-8-oxooctyl (2S)-4-(3-(dimethylamino)propoxy)-1-(6-oxo-6-(undecyloxy)hexyl)pyrrolidine-2-carboxylate |
| 6 | 6-((2-hexyldecanoyl)oxy)hexyl (2S)-1-(6-((2-hexyldecanoyl)oxy)hexyl)-4-((4-hydroxybutyl)amino)pyrrolidine-2-carboxylate |
| 7 | 6-((2-hexyldecanoyl)oxy)hexyl (2S)-1-(6-((2-hexyldecanoyl)oxy)hexyl)-4-((4-hydroxybutyl)(methyl)amino)pyrrolidine-2-carboxylate |
| 8 | 6-((2-hexyldecanoyl)oxy)hexyl (2S)-1-(6-((2-hexyldecanoyl)oxy)hexyl)-4-((3-hydroxypropyl)(methyl)amino)pyrrolidine-2-carboxylate |
| 9 | 6-((2-hexyldecanoyl)oxy)hexyl (2S)-4-(3-(dimethylamino)propoxy)-1-(6-((2-hexyldecanoyl)oxy)hexyl)pyrrolidine-2-carboxylate |
| 10 | 6-((2-hexyldecanoyl)oxy)hexyl (2S)-4-((3-(dimethylamino)propanoyl)oxy)-1-(6-((2-hexyldecanoyl)oxy)hexyl)pyrrolidine-2-carboxylate |
| 11 | 8-(heptadecan-9-yloxy)-8-oxooctyl (2S,4S)-1-(6-oxo-6-(undecyloxy)hexyl)-4-((3-(pyrrolidin-1-yl)propanoyl)oxy)pyrrolidine-2-carboxylate |
| 12. | 8-(heptadecan-9-yloxy)-8-oxooctyl (2S,4S)-4-((3-((S)-3-hydroxypyrrolidin-1-yl)propanoyl)oxy)-1-(6-oxo-6-(undecyloxy)hexyl) |
| | pyrrolidine-2-carboxylate |
| 13. | 8-(heptadecan-9-yloxy)-8-oxooctyl (2S,4S)-4-((3-((R)-3-hydroxypyrrolidin-1-yl)propanoyl)oxy)-1-(6-oxo-6-(undecyloxy)hexyl) |
| | pyrrolidine-2-carboxylate |
| 14. | 2-(((S)-1-(3-(((3S,5S)-5-(((8-(heptadecan-9-yloxy)-8-oxooctyl)oxy)carbonyl)-1-(6-oxo-6-(undecyloxy)hexyl)pyrrolidin-3-yl)oxy)-3- |
| | oxopropyl)pyrrolidin-3-yl)oxy)-N,N,N-trimethyl-2-oxoethan-1-aminium |
| 15. | 2-(((R)-1-(3-(((3S,5S)-5-(((8-(heptadecan-9-yloxy)-8-oxooctyl)oxy)carbonyl)-1-(6-oxo-6-(undecyloxy)hexyl)pyrrolidin-3-yl)oxy)-3- |
| | oxopropyl)pyrrolidin-3-yl)oxy)-N,N,N-trimethyl-2-oxoethan-1-aminium |
| 16. | 8-(heptadecan-9-yloxy)-8-oxooctyl (2S,4S)-4-((4-(4-methylpiperazin-1-yl)-4-oxobutanoyl)oxy)-1-(6-oxo-6-(undecyloxy)hexyl) |
| | pyrrolidine-2-carboxylate |
| 17. | 8-oxo-8-(pentadecan-7-yloxy)octyl (2S,4S)-4-((3-(4-methylpiperazin-1-yl)propanoyl)oxy)-1-(6-oxo-6-(undecyloxy)hexyl)pyrrolidine- |
| | 2-carboxylate |
| 18. | 8-(heptadecan-9-yloxy)-8-oxooctyl (2S,4S)-4-((3-(4-methylpiperazin-1-yl)propanoyl)oxy)-1-(8-oxo-8-((4-pentylnonyl)oxy)octyl) |
| | pyrrolidine-2-carboxylate |
| 19. | 8-(heptadecan-9-yloxy)-8-oxooctyl (2S,4S)-4-(2-(4-methylpiperazin-1-yl)acetoxy)-1-(8-oxo-8-((4-pentylnonyl)oxy)octyl)pyrrolidine-2- |
| | carboxylate |
| 20. | 8-(heptadecan-9-yloxy)-8-oxooctyl (2S,4S)-4-(2-(4-methylpiperazin-1-yl)acetoxy)-1-(6-oxo-6-(undecyloxy)hexyl)pyrrolidine- |
| | 2-carboxylate |
| 21. | 8-(heptadecan-9-yloxy)-8-oxooctyl (2S,4S)-4-((4-(4-methylpiperazin-1-yl)butanoyl)oxy)-1-(6-oxo-6-(undecyloxy)hexyl)pyrrolidine- |
| | 2-carboxylate |
| 22. | 8-oxo-8-((4-pentylnonyl)oxy)octyl (2S,4S)-4-((3-(4-methylpiperazin-1-yl)propanoyl)oxy)-1-(8-oxo-8-((4-pentylnonyl)oxy)octyl) |
| | pyrrolidine-2-carboxylate |
| 23. | 8-oxo-8-((4-pentylnonyl)oxy)octyl (2S,4S)-4-(2-(4-methylpiperazin-1-yl)acetoxy)-1-(8-oxo-8-((4-pentylnonyl)oxy)octyl) |
| | pyrrolidine-2-carboxylate |
| 24. | 8-(heptadecan-9-yloxy)-8-oxooctyl (2S,4S)-1-(6-(heptadecan-9-yloxy)-6-oxohexyl)-4-((3-(4-methylpiperazin-1-yl)propanoyl)oxy) |
| | pyrrolidine-2-carboxylate |
| 25. | 8-(heptadecan-9-yloxy)-8-oxooctyl (2S,4S)-1-(6-(heptadecan-9-yloxy)-6-oxohexyl)-4-(2-(4-methylpiperazin-1-yl)acetoxy) |
| | pyrrolidine-2-carboxylate |
| 26. | 8-oxo-8-(tridecan-7-yloxy)octyl (2S)-4-((3-(dimethylamino)propanoyl)oxy)-1-(6-oxo-6-(tridecan-7-yloxy)hexyl)pyrrolidine-2-carboxylate |
| 27. | 7-(heptadecan-9-yloxy)-7-oxoheptyl (2S)-4-((3-(dimethylamino)propanoyl)oxy)-1-(6-oxo-6-(undecyloxy)hexyl)pyrrolidine-2-carboxylate |
| 28. | 6-(heptadecan-9-yloxy)-6-oxohexyl (2S)-4-((3-(dimethylamino)propanoyl)oxy)-1-(6-oxo-6-(undecyloxy)hexyl)pyrrolidine-2-carboxylate |
| 29. | 8-(heptadecan-9-yloxy)-8-oxooctyl (2S)-4-((3-(dimethylamino)propanoyl)oxy)-1-(5-oxo-5-(undecyloxy)pentyl)pyrrolidine-2-carboxylate |
| 30. | 6-(heptadecan-9-yloxy)-6-oxohexyl (2S)-4-((3-(dimethylamino)propanoyl)oxy)-1-(6-oxo-6-(undecyloxy)hexyl)pyrrolidine-2-carboxylate |
| 31. | 8-(heptadecan-9-yloxy)-8-oxooctyl (2S)-4-((3-(dimethylamino)propanoyl)oxy)-1-(4-oxo-4-(undecyloxy)butyl)pyrrolidine-2-carboxylate |
| 32. | 8-(heptadecan-9-yloxy)-8-oxooctyl (2S)-4-((3-(dimethylamino)propanoyl)oxy)-1-(6-(nonyloxy)-6-oxohexyl)pyrrolidine-2-carboxylate |
| 33. | 8-(heptadecan-9-yloxy)-8-oxooctyl (2S)-4-((3-(dimethylamino)propanoyl)oxy)-1-(6-(heptyloxy)-6-oxohexyl)pyrrolidine-2-carboxylate |

Ionizable Lipid Compounds iii)

In some embodiments, the ionizable lipid is represented by formula

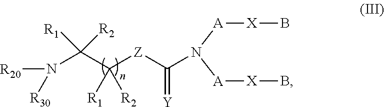

pharmaceutically acceptable salts thereof, and stereoisomers of any of the foregoing, wherein:
R₂₀ and R₃₀ are each independently H, C₁-C₅ branched or unbranched alkyl, or 02-05 branched or unbranched alkenyl, or
R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or
R₁₀ and R₁ᵢ are taken together to form a heterocyclic ring;
n is 0, 1, 2, 3 or 4;
Y is O or S;
Z is absent, O, S, or N(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen;
each X is independently a biodegradable moiety.

In some embodiments, R₂₀ and R₃₀ are each independently H or C₁-C₃ branched or unbranched alkyl. In some embodiments, R₂₀ and R₃₀ together with the adjacent N atom form a 3 to 7 membered cyclic ring, optionally substituted with R^{a}. In some embodiments, R^{a} is H, C₁-C₃ branched or unbranched alkyl or OH. In one embodiment, R^{a} is H or OH.

In some embodiments, Z is absent, S, O, or NH. In some embodiments, n is 0, 1, or 2.

In some embodiments, the ionizable lipid is represented by formula (V):

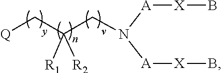

pharmaceutically acceptable salts thereof, and stereoisomers of any of the foregoing, wherein:
R₁ is H, C₁-C₃ branched or unbranched alkyl, C₂-C₃ branched or unbranched alkenyl, OH, halogen, SH, or NR₁₀R₁₁, and
R₂ is H, OH, halogen, SH, or NR₁₀R₁₁, or
R₁ and R₂ are taken together to form a cyclic ring;
R₁₀ and R₁₁ are each independently H or C₁-C₃ alkyl, or R₁₀ and R₁₁ are taken together to form a heterocyclic ring;
Q is OH or —(OCH₂CH₂)ᵤNR₂₀R₃₀,
R₂₀ and R₃₀ are each independently H, C₁-C₅ branched or unbranched alkyl, or C₂-C₅ branched or unbranched alkenyl, or
R₂₀ and R₃₀ together with the adjacent N atom form a 3 to 7 membered cyclic ring optionally substituted with R^{a}
R^{a} is H, C₁-C₃ branched or unbranched alkyl, C₂-C₃ branched or unbranched alkenyl, halogen, OH, or SH;
v is 0, 1, 2, 3, or 4;
y is 0, 1, 2, 3, or 4;
each A is independently C₁-C₁₆ branched or unbranched alkyl or C₁-C₁₆ branched or unbranched alkenyl, optionally interrupted with one or more heteroatoms or optionally substituted with OH, SH, or halogen;
each B is each independently C₁-C₁₆ branched or unbranched alkyl or C₂-C₁₆ branched or unbranched alkenyl, optionally interrupted with one or more heteroatoms or optionally substituted with OH, SH, or halogen; and
each X is independently a biodegradable moiety.

In some embodiments, the disclosure relates to ionizable lipids of one of the following formulas:

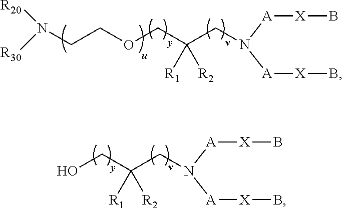

wherein: u is 0, 1, 2, 3, 4, 5, 6, 7, or 8; v is 0, 1, 2, 3, or 4; and y is 0, 1, 2, 3, or 4. Other variables are defined as in formulas III) and V) above.

In some embodiments, in the above formulas, X is —OC(O)—, —C(O)O—, —N(R⁷)C(O)—, —C(O)N(R₇)—,

—C(O—R₁₃)—O—, —C(O)O(CH₂)ₛ—, —OC(O)(CH₂)ₛ—, —C(O)N(R⁷)(CH₂)ₛ—, —N(R⁷)C(O)(CH₂)ₛ—, —C(O—R₁₃)—O—(CH₂)ₛ—, wherein each R⁷ is independently H, alkyl, alkenyl, cycloalkyl, hydroxyalkyl, or aminoalkyl, each R₁₃ is independently C₃-C₁₀ alkyl, and each s is independently 0-16. In some embodiments, X is —OC(O)—, —C(O)O—, —C(O)O(CH₂)ₛ—, or —OC(O)(CH₂)ₛ—. In some embodiments, s is 0, 1, 2, 3, 4, 5, 6, 7, 8, 9, or 10.

More embodiments of the ionizable lipid of formula (III) or (V), in the Ionizable lipid compounds group iii), may be found in PCT Application No. PCT/US22/50111, filed on Nov. 16, 2022, the content of which is incorporated herein by reference in its entirety. In particular, all the ionizable lipids of formulas (IO)—(VIIO) and formulas (I)—(VIID) of PCT Application No. PCT/US22/50111 are suitable for use as the ionizable lipids in this disclosure, and are incorporated herein by reference in its entirety.

Certain exemplary ionizable lipid compounds disclosed herein are set forth in Table III below.

TABLE III
Exemplary ionizable lipid compounds.

| Lipid | | |
|---|---|---|
| No. | Structure | Name |
| 2140 | 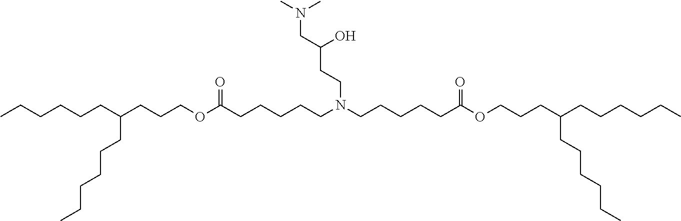 [SMILES: CCCCCCC(CCCCCC)CCCOC(=O)CCCCCN(CCCCCC(=O)OCCCC(CCCCCC)CCCCCC)CCC(O)CN(C)C] | bis(4-hexyldecyl) 6,6′- ((4-(dimethylamino)- 3-hydroxybutyl) azanediyl) dihexanoate |
| 2139 | 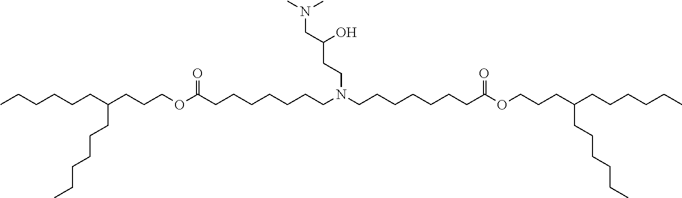 [SMILES: CCCCCCC(CCCCCC)CCCOC(=O)CCCCCCCN(CCCCCCCC(=O)OCCCC(CCCCCC)CCCCCC)CCC(O)CN(C)C] | bis(4-hexyldecyl) 8,8′- ((4-(dimethylamino)- 3-hydroxybutyl) azanediyl)dioctanoate |
| 2138 | 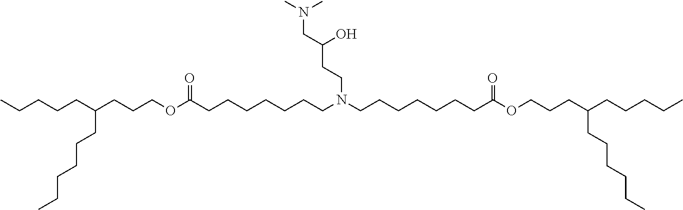 [SMILES: CCCCCCC(CCCCC)CCCOC(=O)CCCCCCCN(CCCCCCCC(=O)OCCCC(CCCCC)CCCCCC)CCC(O)CN(C)C] | bis(4-pentylnonyl) 8,8′-((4- (dimethylamino)-3- hydroxybutyl) azanediyl)dioctanoate |
| 2142 | 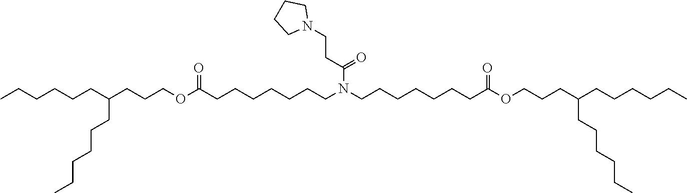 [SMILES: CCCCCCC(CCCCCC)CCCOC(=O)CCCCCCCN(CCCCCCCC(=O)OCCCC(CCCCCC)CCCCCC)C(=O)CCN1CCCC1] | bis(4-hexyldecyl) 8,8′- ((3-(pyrrolidin-1-yl) propanoyl)azanediyl) dioctanoate |
| 2146 | 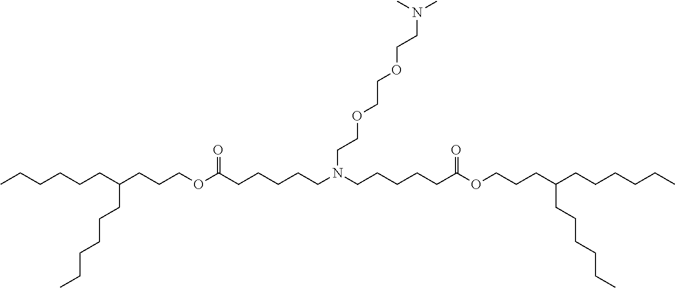 [SMILES: CCCCCCC(CCCCCC)CCCOC(=O)CCCCCN(CCCCCC(=O)OCCCC(CCCCCC)CCCCCC)CCOCCOCCN(C)C] | 4-hexyldecyl 11-(6- ((4-hexyldecyl)oxy)- 6-oxohexyl)-2- methyl-5,8-dioxa- 2,11-diazaheptadecan- 17-oate |
| 2145 | 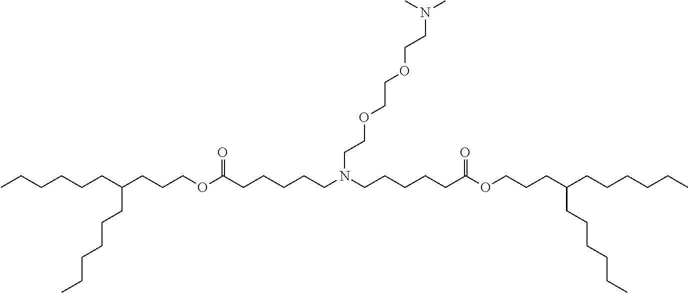 [SMILES: CCCCCCC(CCCCCC)CCCOC(=O)CCCCCN(CCCCCC(=O)OCCCC(CCCCCC)CCCCCC)CCOCCOCCN(C)C] | 4-hexyldecyl 11-(8- ((4-hexyldecyl)oxy)- 8-oxooctyl)-2-methyl- 5,8-dioxa-2,11- diazanonadecan-19- oate |
| 2133 | 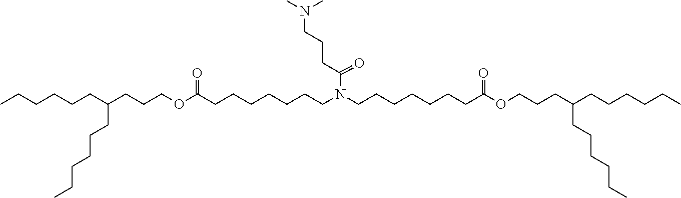 [SMILES: CCCCCCC(CCCCCC)CCCOC(=O)CCCCCCCN(CCCCCCCC(=O)OCCCC(CCCCCC)CCCCCC)C(=O)CCCN(C)C] | bis(4-hexyldecyl) 8,8′-((4- (dimethylamino) butanoyl)azanediyl) dioctanoate |
| 2137 | 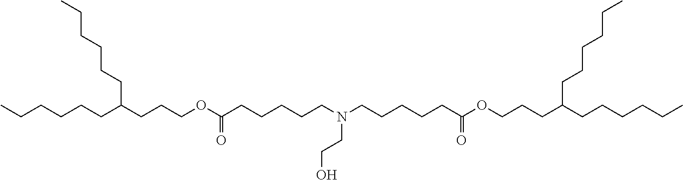 [SMILES: CCCCCCC(CCCCCC)CCCOC(=O)CCCCCN(CCO)CCCCCC(=O)OCCCC(CCCCCC)CCCCCC] | bis(4-hexyldecyl) 6,6′-((2-hydroxy- ethyl)azanediyl) dihexanoate |
| 2131 | 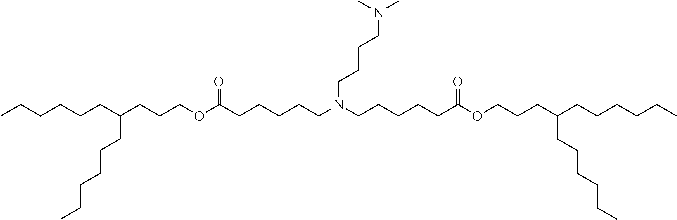 [SMILES: CCCCCCC(CCCCCC)CCCOC(=O)CCCCCN(CCCCCC(=O)OCCCC(CCCCCC)CCCCCC)CCCCN(C)C] | bis(4-hexyldecyl) 6,6′- ((4-(dimethylamino) butyl)azanediyl) dihexanoate |
| 2130 | 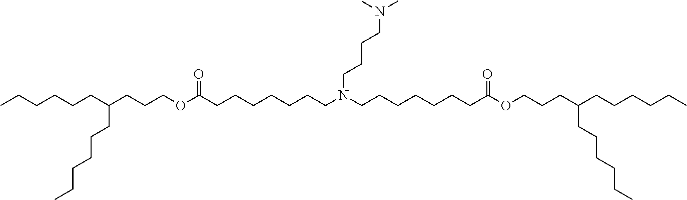 [SMILES: CCCCCCC(CCCCCC)CCCOC(=O)CCCCCCCN(CCCCCCCC(=O)OCCCC(CCCCCC)CCCCCC)CCCCN(C)C] | bis(4-hexyldecyl) 8,8′- ((4-(dimethylamino) butyl)azanediyl) dioctanoate |
| 2129 | 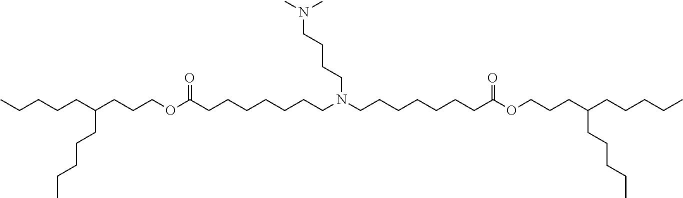 [SMILES: CCCCCC(CCCCC)CCCOC(=O)CCCCCCCN(CCCCCCCC(=O)OCCCC(CCCCC)CCCCC)CCCCN(C)C] | bis(4-pentylnonyl) 8,8′-((4- (dimethylamino) butyl)azanediyl) dioctanoate |
| 2144 | 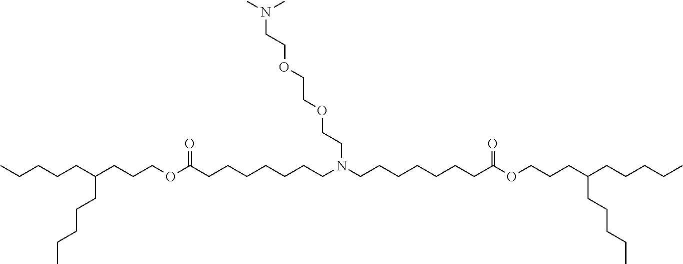 [SMILES: CCCCCC(CCCCC)CCCOC(=O)CCCCCCCN(CCCCCCCC(=O)OCCCC(CCCCC)CCCCC)CCOCCOCCN(C)C] | 4-pentylnonyl 2- methyl-11-(8-oxo-8- ((4-pentylnonyl)oxy) octyl)-5,8-dioxa-2,11- diazanonadecan-19- oate |
| 2143 | 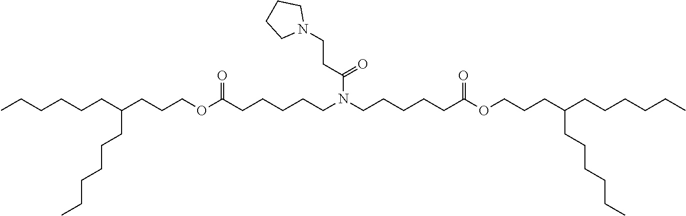 [SMILES: CCCCCCC(CCCCCC)CCCOC(=O)CCCCCN(CCCCCC(=O)OCCCC(CCCCCC)CCCCCC)C(=O)CCN1CCCC1] | bis(4-hexyldecyl) 6,6′-((3-(pyrrolidin-1- yl)propanoyl) azanediyl) dihexanoate |
| 2134 | 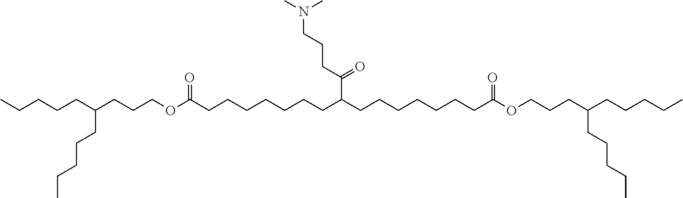 [SMILES: CCCCCC(CCCCC)CCCOC(=O)CCCCCCCC(CCCCCCCC(=O)OCCCC(CCCCC)CCCCC)C(=O)CCCN(C)C] | bis(4-hexyldecyl) 6,6′-((4- (dimethylamino) butanoyl)azanediyl) dihexanoate |
| 2135 | 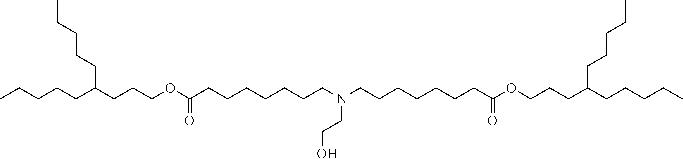 [SMILES: CCCCCC(CCCCC)CCCOC(=O)CCCCCCCN(CCO)CCCCCCCC(=O)OCCCC(CCCCC)CCCCC] | bis(4-pentylnonyl) 8,8′-((2-hydroxy- ethyl)azanediyl) dioctanoate |
| 2136 | 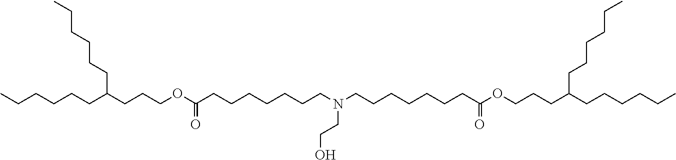 [SMILES: CCCCCCC(CCCCCC)CCCOC(=O)CCCCCCCN(CCO)CCCCCCCC(=O)OCCCC(CCCCCC)CCCCCC] | bis(4-hexyldecyl) 8,8′-((2-hydroxyethyl) azanediyl) dioctanoate |
| 2141 | 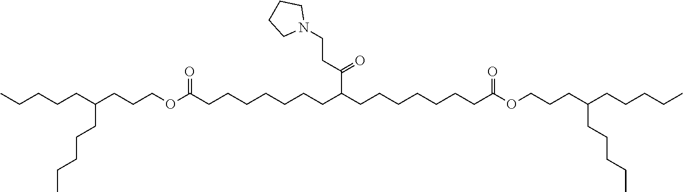 [SMILES: CCCCCC(CCCCC)CCCOC(=O)CCCCCCCC(CCCCCCCC(=O)OCCCC(CCCCC)CCCCC)C(=O)CCN1CCCC1] | bis(4-pentylnonyl) 8,8′-((3-(pyrrolidin-1- yl)propanoyl) azanediyl)dioctanoate |
| 2132 | 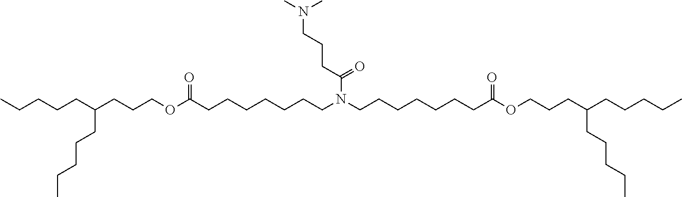 [SMILES: CCCCCC(CCCCC)CCCOC(=O)CCCCCCCN(CCCCCCCC(=O)OCCCC(CCCCC)CCCCC)C(=O)CCCN(C)C] | bis(4-pentylnonyl) 8,8′-((4- (dimethylamino) butanoyl)azanediyl) dioctanoate |
| 2229 | 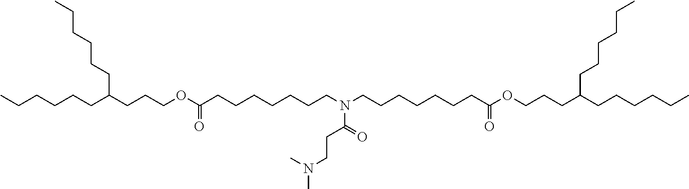 [SMILES: CCCCCCC(CCCCCC)CCCOC(=O)CCCCCCCN(CCCCCCCC(=O)OCCCC(CCCCCC)CCCCCC)C(=O)CCN(C)C] | bis(4-hexyldecyl) 8,8′-((3-(dimethyl- amino)propanoyl) azanediyl)dioctanoate |
| 2228 | 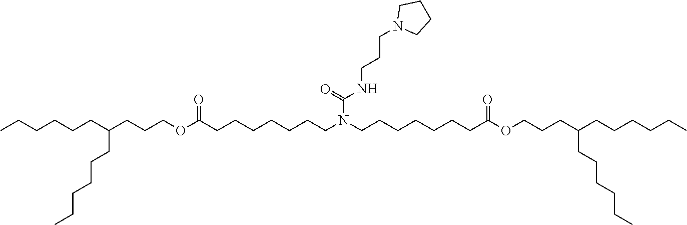 [SMILES: CCCCCCC(CCCCCC)CCCOC(=O)CCCCCCCN(CCCCCCCC(=O)OCCCC(CCCCCC)CCCCCC)C(=O)NCCCN1CCCC1] | bis(4-hexyldecyl) 8,8′-(((3-(pyrrolidin- 1-yl)propyl) carbamoyl) azanediyl)dioctanoate |
| 2227 | 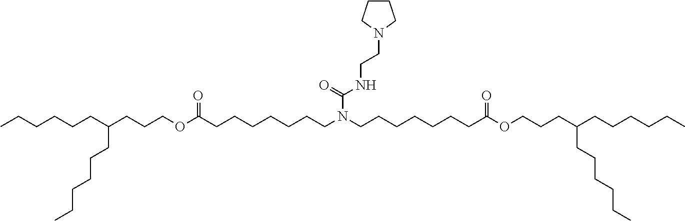 [SMILES: CCCCCCC(CCCCCC)CCCOC(=O)CCCCCCCN(CCCCCCCC(=O)OCCCC(CCCCCC)CCCCCC)C(=O)NCCN1CCCC1] | bis(4-hexyldecyl) 8,8′-(((2-(pyrrolidin- 1-yl)ethyl)carbamoyl) azanediyl)dioctanoate |
| 2226 | 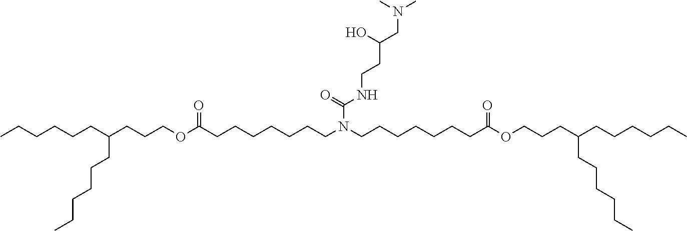 [SMILES: CCCCCCC(CCCCCC)CCCOC(=O)CCCCCCCN(CCCCCCCC(=O)OCCCC(CCCCCC)CCCCCC)C(=O)NCCC(O)CN(C)C] | bis(4-hexyldecyl) 8,8′-(((4- (dimethylamino)-3- hydroxybutyl) carbamoyl) azanediyl)dioctanoate |
| 2225 | 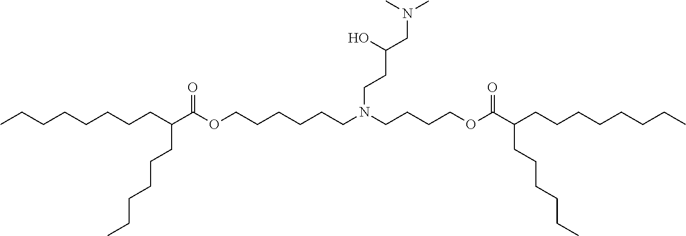 [SMILES: CCCCCCCCC(CCCCCC)C(=O)OCCCCCCN(CCCCOC(=O)C(CCCCCC)CCCCCCCC)CCC(O)CN(C)C] | ((4-(dimethylamino)- 3-hydroxybutyl) azanediyl)bis(hexane- 6,1-diyl) bis(2- hexyldecanoate |
| 2216 | 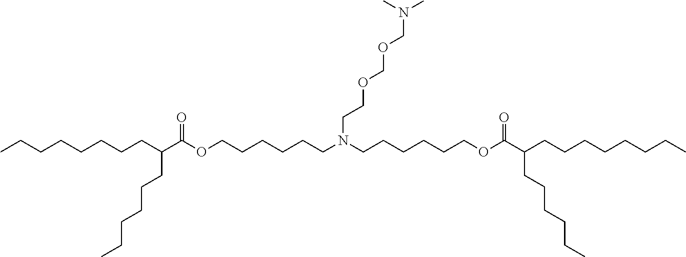 [SMILES: CCCCCCCCC(CCCCCC)C(=O)OCCCCCCN(CCCCCCOC(=O)C(CCCCCC)CCCCCCCC)CCOCOCN(C)C] | 11-(6-((2- hexyldecanoyl)oxy) hexyl)-2-methyl-5,8- dioxa-2,11-diaza- heptadecan-17-yl 2- hexyldecanoate |
| 2215 | 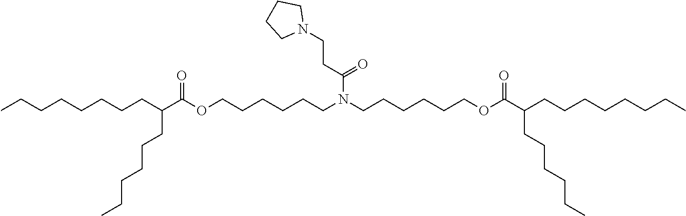 [SMILES: CCCCCCCCC(CCCCCC)C(=O)OCCCCCCN(CCCCCCOC(=O)C(CCCCCC)CCCCCCCC)C(=O)CCN1CCCC1] | ((3-(pyrrolidin-1- yl)propanoyl) azanediyl)bis(hexane- 6,1-diyl) bis(2- hexyldecanoate) |
| | 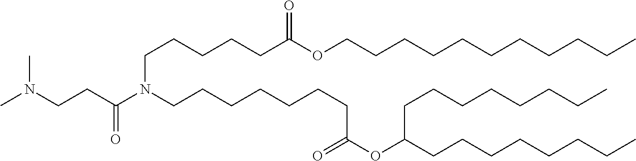 [SMILES: CCCCCCCCCCCOC(=O)CCCCCN(CCCCCCCC(=O)OC(CCCCCCCC)CCCCCCCC)C(=O)CCN(C)C] | heptadecan-9-yl 8-(3- (dimethylamino)-N- (6-oxo-6- (undecyloxy) hexyl)propanamido) octanoate |
| | 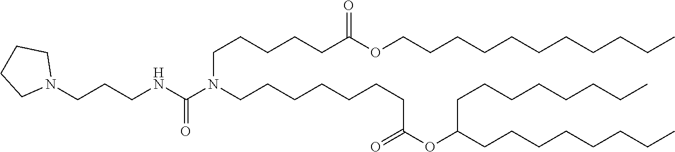 [SMILES: CCCCCCCCCCCOC(=O)CCCCCN(CCCCCCCC(=O)OC(CCCCCCCC)CCCCCCCC)C(=O)NCCCN1CCCC1] | heptadecan-9-yl 8-(1- (6-oxo-6- (undecyloxy)hexyl)-3- (3-(pyrrolidin-1- yl)propyl)ureido) octanoate |
| | 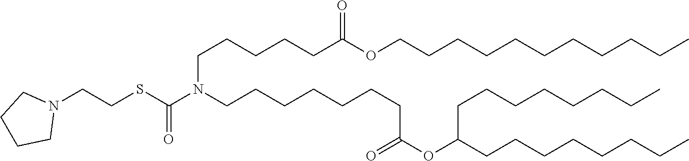 [SMILES: CCCCCCCCCCCOC(=O)CCCCCN(CCCCCCCC(=O)OC(CCCCCCCC)CCCCCCCC)C(=O)SCCN1CCCC1] | heptadecan-9-yl 8- ((6-oxo-6- (undecyloxy)hexyl) (((2-(pyrrolidin-1- yl)ethyl)thio) carbonyl)amino) octanoate |
| | 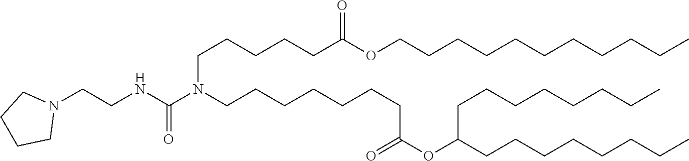 [SMILES: CCCCCCCCCCCOC(=O)CCCCCN(CCCCCCCC(=O)OC(CCCCCCCC)CCCCCCCC)C(=O)NCCN1CCCC1] | heptadecan-9-yl 8-(1- (6-oxo-6- (undecyloxy)hexyl)-3- (2-(pyrrolidin-1- yl)ethyl)ureido) octanoate |
| | 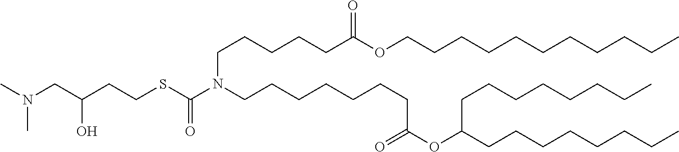 [SMILES: CCCCCCCCCCCOC(=O)CCCCCN(CCCCCCCC(=O)OC(CCCCCCCC)CCCCCCCC)C(=O)SCCC(O)CN(C)C] | heptadecan-9-yl 8- ((((4-(dimethyl- amino)-3-hydroxy- butyl)thio)carbonyl) (6-oxo-6- (undecyloxy)hexyl) amino)octanoate |
| | 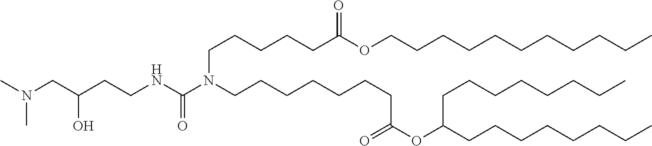 [SMILES: CCCCCCCCCCCOC(=O)CCCCCN(CCCCCCCC(=O)OC(CCCCCCCC)CCCCCCCC)C(=O)NCCC(O)CN(C)C] | heptadecan-9-yl 8-(3- (4-(dimethylamino)-3- hydroxybutyl)-1-(6- oxo-6-(undecyloxy) hexyl)ureido) octanoate |
| | 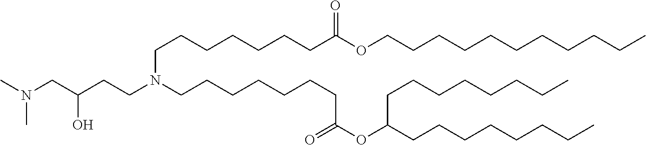 [SMILES: CCCCCCCCCCCOC(=O)CCCCCCCN(CCCCCCCC(=O)OC(CCCCCCCC)CCCCCCCC)CCC(O)CN(C)C] | heptadecan-9-yl 8- ((4-(dimethylamino)- 3-hydroxybutyl) (6-oxo-6- (undecyloxy)hexyl) amino)octanoate |
| | 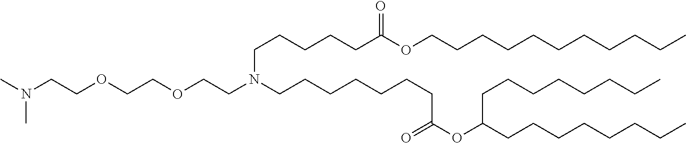 [SMILES: CCCCCCCCCCCOC(=O)CCCCCN(CCCCCCCC(=O)OC(CCCCCCCC)CCCCCCCC)CCOCCOCCN(C)C] | heptadecan-9-yl 2- methyl-11-(6-oxo-6- (undecyloxy)hexyl)- 5,8-dioxa-2,11- diazanonadecan-19- oate |
| | 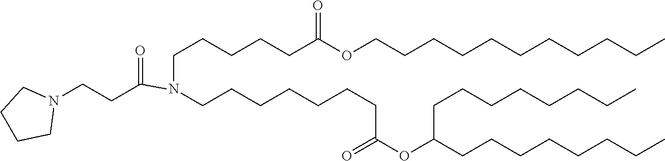 [SMILES: CCCCCCCCCCCOC(=O)CCCCCN(CCCCCCCC(=O)OC(CCCCCCCC)CCCCCCCC)C(=O)CCN1CCCC1] | heptadecan-9-yl 8- (N-(6-oxo-6- (undecyloxy)hexyl)- 3-(pyrrolidin-1- yl)propanamido) octanoate |
| | 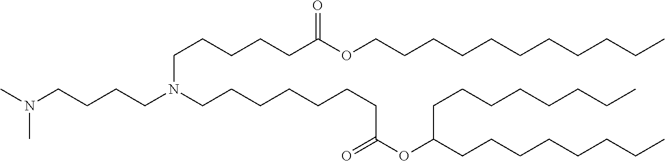 [SMILES: CCCCCCCCCCCOC(=O)CCCCCN(CCCCCCCC(=O)OC(CCCCCCCC)CCCCCCCC)CCCCN(C)C] | heptadecan-9-yl 8- ((4-(dimethylamino) butyl)(6-oxo-6- (undecyloxy)hexyl) amino)octanoate |
| | 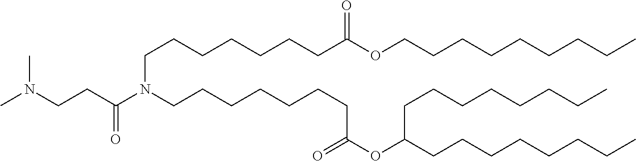 [SMILES: CCCCCCCCCOC(=O)CCCCCCCN(CCCCCCCC(=O)OC(CCCCCCCC)CCCCCCCC)C(=O)CCN(C)C] | heptadecan-9-yl 8-(3- (dimethylamino)-N- (8-(nonyloxy)-8- oxooctyl) propanamido) octanoate |
| | 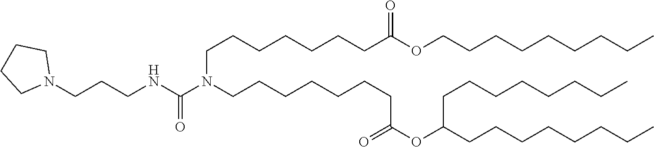 [SMILES: CCCCCCCCCOC(=O)CCCCCCCN(CCCCCCCC(=O)OC(CCCCCCCC)CCCCCCCC)C(=O)NCCCN1CCCC1] | heptadecan-9-yl 8-(1- (8-(nonyloxy)-8- oxooctyl)-3-(3- (pyrrolidin-1-yl) propyl)ureido) octanoate |
| | 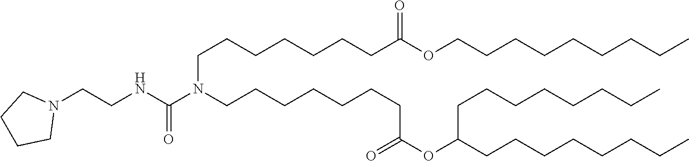 [SMILES: CCCCCCCCCOC(=O)CCCCCCCN(CCCCCCCC(=O)OC(CCCCCCCC)CCCCCCCC)C(=O)NCCN1CCCC1] | heptadecan-9-yl 8-(1- (8-(nonyloxy)-8- oxooctyl)-3-(2- (pyrrolidin-1-yl) ethyl)ureido)octanoate |
| | 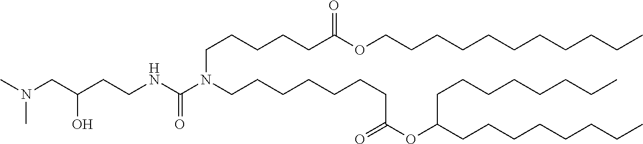 [SMILES: CCCCCCCCCCCOC(=O)CCCCCN(CCCCCCCC(=O)OC(CCCCCCCC)CCCCCCCC)C(=O)NCCC(O)CN(C)C] | heptadecan-9-yl 8-(3- (4-(dimethylamino)- 3-hydroxybutyl)-1-(8- (nonyloxy)-8- oxooctyl)ureido) octanoate |
| | 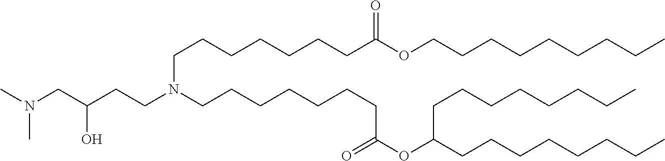 [SMILES: CCCCCCCCCOC(=O)CCCCCCCN(CCCCCCCC(=O)OC(CCCCCCCC)CCCCCCCC)CCC(O)CN(C)C] | heptadecan-9-yl 8- ((4-(dimethylamino)- 3-hydroxybutyl)(8- (nonyloxy)-8- oxooctyl)amino) octanoate |
| | 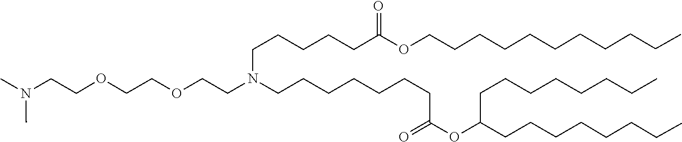 [SMILES: CCCCCCCCCCCOC(=O)CCCCCN(CCCCCCCC(=O)OC(CCCCCCCC)CCCCCCCC)CCOCCOCCN(C)C] | heptadecan-9-yl 2- methyl-11-(8- (nonyloxy)-8- oxooctyl)-5,8-dioxa- 2,11-diazanonadecan- 19-oate |
| | 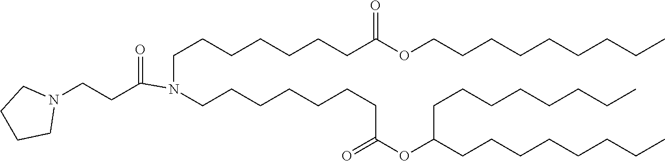 [SMILES: CCCCCCCCCOC(=O)CCCCCCCN(CCCCCCCC(=O)OC(CCCCCCCC)CCCCCCCC)C(=O)CCN1CCCC1] | heptadecan-9-yl 8- (N-(8-(nonyloxy)- 8-oxooctyl)-3- (pyrrolidin-1-yl) propanamido) octanoate |
| | 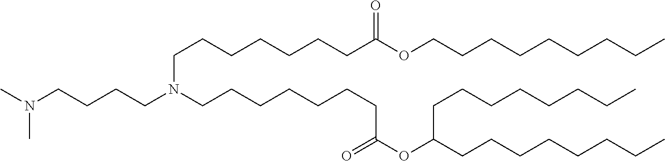 [SMILES: CCCCCCCCCOC(=O)CCCCCCCN(CCCCCCCC(=O)OC(CCCCCCCC)CCCCCCCC)CCCCN(C)C] | heptadecan-9-yl 8- ((4-(dimethylamino) butyl)(8-(nonyloxy)- 8-oxooctyl)amino) octanoate |
| | 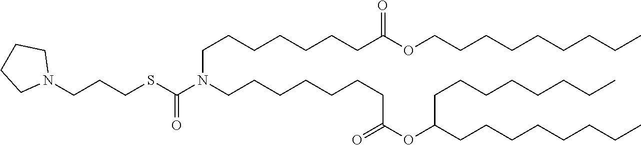 [SMILES: CCCCCCCCCOC(=O)CCCCCCCN(CCCCCCCC(=O)OC(CCCCCCCC)CCCCCCCC)C(=O)SCCCN1CCCC1] | heptadecan-9-yl 8- ((8-(nonyloxy)-8- oxooctyl)(((3- (pyrrolidin-1-yl) propyl)thio) carbonyl)amino) octanoate |
| | 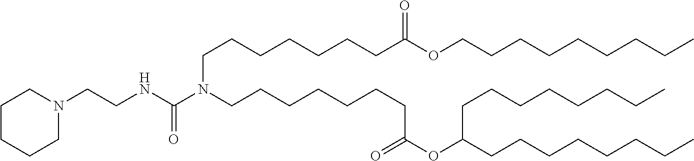 [SMILES: CCCCCCCCCOC(=O)CCCCCCCN(CCCCCCCC(=O)OC(CCCCCCCC)CCCCCCCC)C(=O)NCCN1CCCCC1] | heptadecan-9-yl 8- (1-(8-(nonyloxy)-8- oxooctyl)-3-(2- (piperidin-1- yl)ethyl)ureido) octanoate |
| | 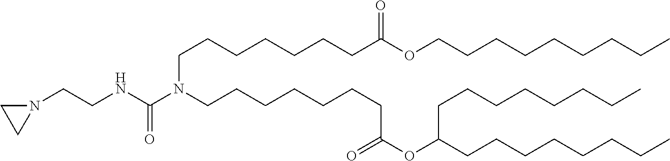 [SMILES: CCCCCCCCCOC(=O)CCCCCCCN(CCCCCCCC(=O)OC(CCCCCCCC)CCCCCCCC)C(=O)NCCN1CC1] | heptadecan-9-yl 8-(3- (2-(aziridin-1-yl) ethyl)-1-(8- (nonyloxy)-8- oxooctyl)ureido) octanoate |
| | 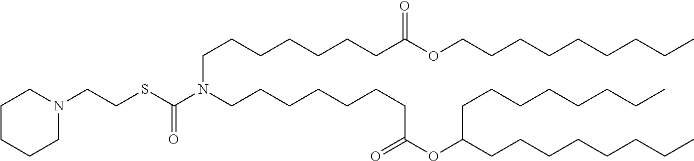 [SMILES: CCCCCCCCCOC(=O)CCCCCCCN(CCCCCCCC(=O)OC(CCCCCCCC)CCCCCCCC)C(=O)SCCN1CCCCC1] | heptadecan-9-yl 8- ((8-(nonyloxy)-8- oxooctyl)(((2- (piperidin-1-yl)ethyl) thio)carbonyl) amino)octanoate |
| | 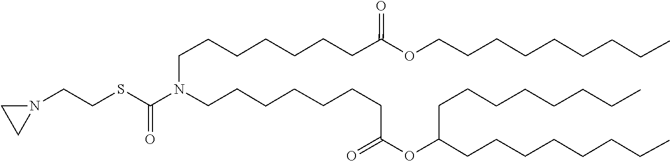 [SMILES: CCCCCCCCCOC(=O)CCCCCCCN(CCCCCCCC(=O)OC(CCCCCCCC)CCCCCCCC)C(=O)SCCN1CC1] | heptadecan-9-yl 8- ((((2-(aziridin-1- yl)ethyl)thio) carbonyl)(8- (nonyloxy)-8- oxooctyl)amino) octanoate |
| | 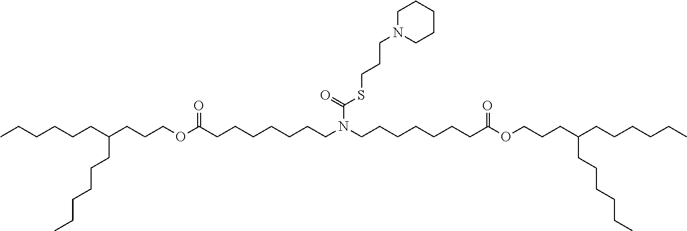 [SMILES: CCCCCCC(CCCCCC)CCCOC(=O)CCCCCCCN(CCCCCCCC(=O)OCCCC(CCCCCC)CCCCCC)C(=O)SCCCN1CCCCC1] | bis(4-hexyldecyl) 8,8′- ((((3-(pyrrolidin-1- yl)propyl)thio) carbonyl)azanediyl) dioctanoate |
| | 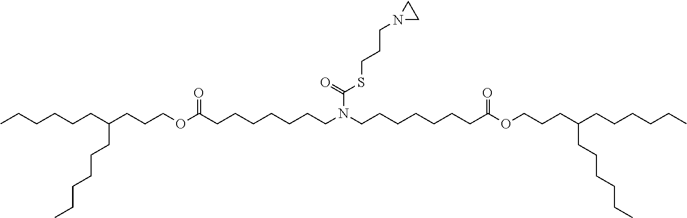 [SMILES: CCCCCCC(CCCCCC)CCCOC(=O)CCCCCCCN(CCCCCCCC(=O)OCCCC(CCCCCC)CCCCCC)C(=O)SCCCN1CC1] | bis(4-hexyldecyl) 8,8′- ((((3-(aziridin-1-yl) propyl)thio) carbonyl)azanediyl) dioctanoate |
| | 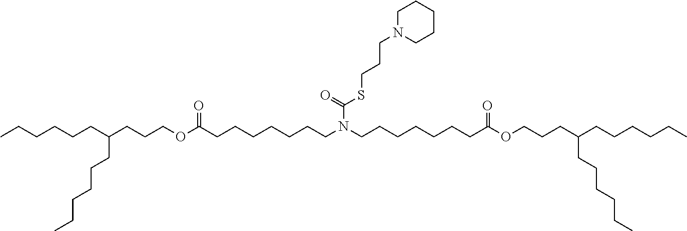 [SMILES: CCCCCCC(CCCCCC)CCCOC(=O)CCCCCCCN(CCCCCCCC(=O)OCCCC(CCCCCC)CCCCCC)C(=O)SCCCN1CCCCC1] | bis(4-hexyldecyl) 8,8′- ((((3-(piperidin-1- yl)propyl)thio) carbonyl)azanediyl) dioctanoate |
| | 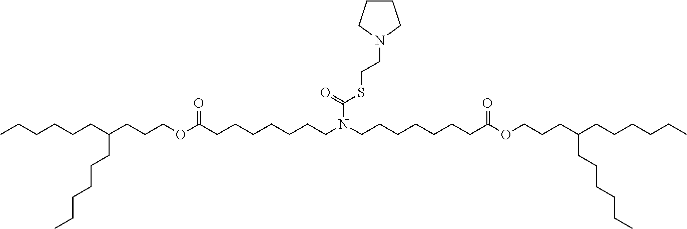 [SMILES: CCCCCCC(CCCCCC)CCCOC(=O)CCCCCCCN(CCCCCCCC(=O)OCCCC(CCCCCC)CCCCCC)C(=O)SCCN1CCCC1] | bis(4-hexyldecyl) 8,8′- ((((2-(pyrrolidin-1- yl)ethyl)thio) carbonyl)azanediyl) dioctanoate |
| | 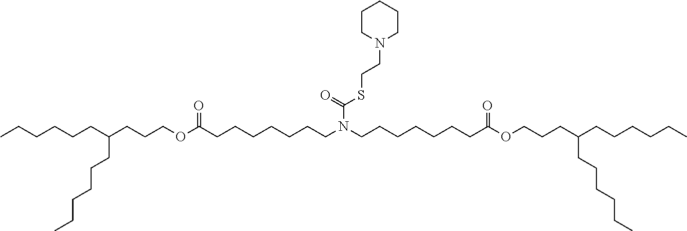 [SMILES: CCCCCCC(CCCCCC)CCCOC(=O)CCCCCCCN(CCCCCCCC(=O)OCCCC(CCCCCC)CCCCCC)C(=O)SCCN1CCCCC1] | bis(4-hexyldecyl) 8,8′- ((((2-(piperidin-1- yl)ethyl)thio) carbonyl)azanediyl) dioctanoate |
| | 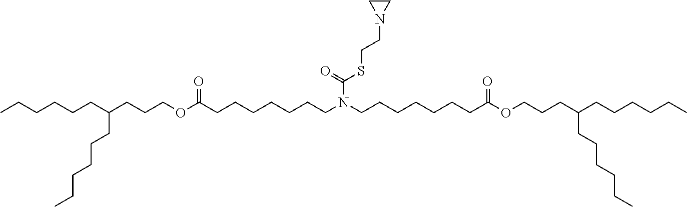 [SMILES: CCCCCCC(CCCCCC)CCCOC(=O)CCCCCCCN(CCCCCCCC(=O)OCCCC(CCCCCC)CCCCCC)C(=O)SCCN1CC1] | bis(4-hexyldecyl) 8,8′- ((((2-(aziridin-1- yl)ethyl)thio) carbonyl)azanediyl) dioctanoate |
| 2233 | 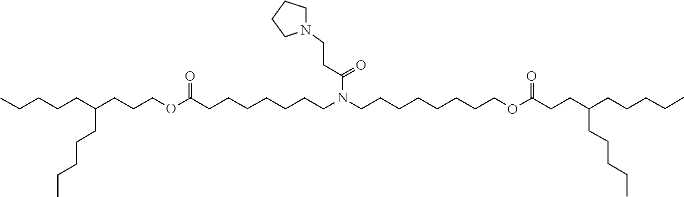 [SMILES: CCCCCC(CCCCC)CCCOC(=O)CCCCCCCN(CCCCCCCCOC(=O)CCC(CCCCC)CCCCC)C(=O)CCN1CCCC1] | bis(4-pentylnonyl) 8,8′-((2-(pyrrolidin-1- yl)acetyl)azanediyl) dioctanoate |
| 2234 | 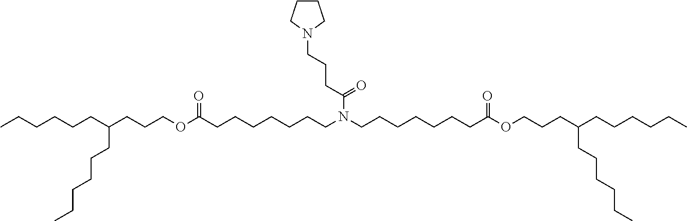 [SMILES: CCCCCCC(CCCCCC)CCCOC(=O)CCCCCCCN(CCCCCCCC(=O)OCCCC(CCCCCC)CCCCCC)C(=O)CCCN1CCCC1] | bis(4-pentylnonyl) 8,8′-((4-(pyrrolidin-1- yl)butanoyl) azanediyl) dioctanoate |
| 2235 | 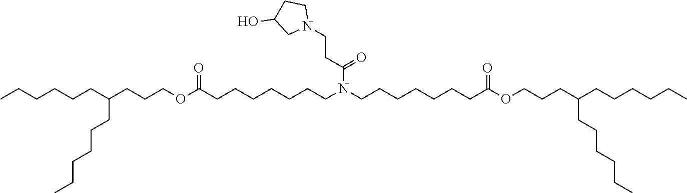 [SMILES: CCCCCCC(CCCCCC)CCCOC(=O)CCCCCCCN(CCCCCCCC(=O)OCCCC(CCCCCC)CCCCCC)C(=O)CCN1CCC(O)C1] | bis(4-pentylnonyl) 8,8′-((3-(3-hydroxy- pyrrolidin-1-yl) propanoyl)azanediyl) dioctanoate |
| 2236 | 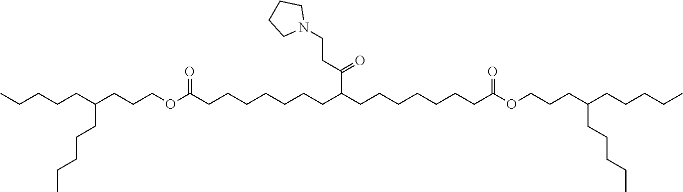 [SMILES: CCCCCC(CCCCC)CCCOC(=O)CCCCCCCC(CCCCCCCC(=O)OCCCC(CCCCC)CCCCC)C(=O)CCN1CCCC1] | bis(4-pentylnonyl) 8,8′-((3-(pyrrolidin-1- yl)propanethioyl) azanediyl)dioctanoate |
| 2237 | 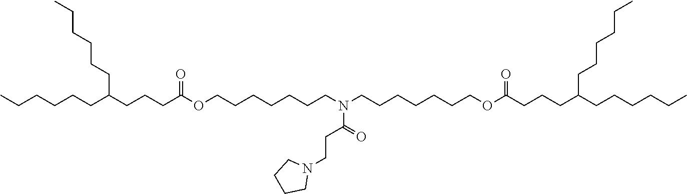 [SMILES: CCCCCCC(CCCCCC)CCCC(=O)OCCCCCCCN(CCCCCCCOC(=O)CCCC(CCCCCC)CCCCCC)C(=O)CCN1CCCC1] | ((3-(pyrrolidin-1-yl) propanoyl)azanediyl) bis(heptane-7,1-diyl) bis(5- pentyldecanoate) |
| 2238 | 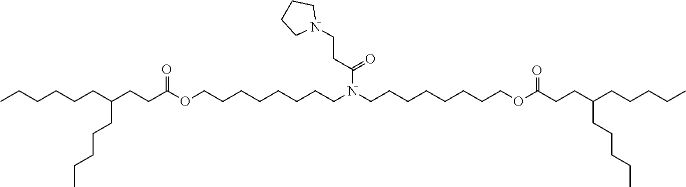 [SMILES: CCCCCCC(CCCCC)CCC(=O)OCCCCCCCCN(CCCCCCCCOC(=O)CCC(CCCCC)CCCCC)C(=O)CCN1CCCC1] | ((3-(pyrrolidin-1-yl) propanoyl)azanediyl) bis(octane-8,1-diyl) bis(4- pentylnonanoate) |
| 2239 | 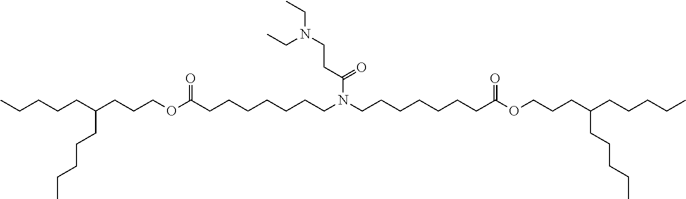 [SMILES: CCCCCC(CCCCC)CCCOC(=O)CCCCCCCN(CCCCCCCC(=O)OCCCC(CCCCC)CCCCC)C(=O)CCN(CC)CC] | bis(4-pentylnonyl) 8,8′-((3- (diethylamino) propanoyl)azanediyl) dioctanoate |
| 2241 | 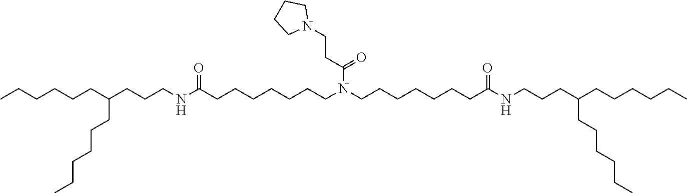 [SMILES: CCCCCCC(CCCCCC)CCCNC(=O)CCCCCCCN(CCCCCCCC(=O)NCCCC(CCCCCC)CCCCCC)C(=O)CCN1CCCC1] | 8-(N-(8-oxo-8-((4- pentylnonyl)amino) octyl)-3-(pyrrolidin- 1-yl)propanamido)-N- (4-pentylnonyl) octanamide |
| 2242 | 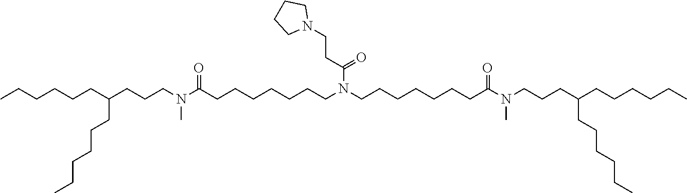 [SMILES: CCCCCCC(CCCCCC)CCCN(C)C(=O)CCCCCCCN(CCCCCCCC(=O)N(C)CCCC(CCCCCC)CCCCCC)C(=O)CCN1CCCC1] | N-methyl-8-(N-(8- (methyl(4- pentylnonyl)amino)- 8-oxooctyl)-3- (pyrrolidin-1-yl) propanamido)-N-(4- pentylnonyl) octanamide |
| 2244 | 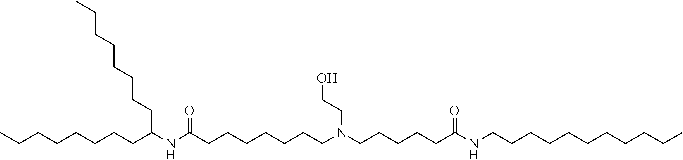 [SMILES: CCCCCCCCCCCNC(=O)CCCCCN(CCO)CCCCCCCC(=O)NC(CCCCCCCC)CCCCCCCC] | N-(heptadecan-9-yl)- 8-((2-hydroxyethyl) (6-oxo-6- (undecylamino) hexyl)amino) octanamide |
| 2245 | 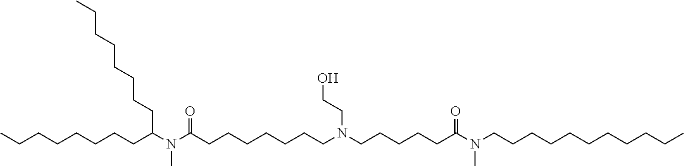 [SMILES: CCCCCCCCCCCN(C)C(=O)CCCCCN(CCO)CCCCCCCC(=O)N(C)C(CCCCCCCC)CCCCCCCC] | N-(heptadecan-9-yl)- 8-((2-hydroxyethyl) (6-(methyl(undecyl) amino)-6-oxohexyl) amino)-N-methyl- octanamide |
| 2249 | 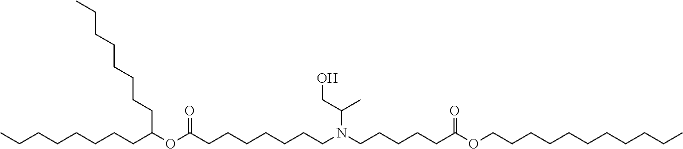 [SMILES: CCCCCCCCCCCOC(=O)CCCCCN(CCCCCCCC(=O)OC(CCCCCCCC)CCCCCCCC)C(C)CO] | heptadecan-9-yl 8-((1- hydroxypropan-2-yl) (6-oxo-6-(undecyl- oxy)hexyl)amino) octanoate |
| 2250 | 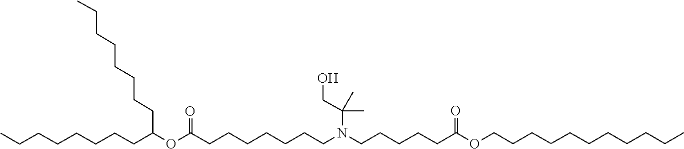 [SMILES: CCCCCCCCCCCOC(=O)CCCCCN(CCCCCCCC(=O)OC(CCCCCCCC)CCCCCCCC)C(C)(C)CO] | heptadecan-9-yl 8-((1- hydroxy-2-methyl- propan-2-yl)(6-oxo-6- (undecyloxy)hexyl) amino)octanoate |
| 2276 | 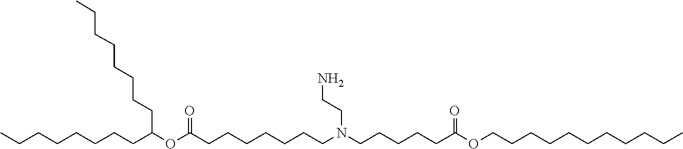 [SMILES: CCCCCCCCCCCOC(=O)CCCCCN(CCN)CCCCCCCC(=O)OC(CCCCCCCC)CCCCCCCC] | heptadecan-9-yl 8-((2- aminoethyl)(6-oxo-6- (undecyloxy)hexyl) amino)octanoate |
| 2277 | 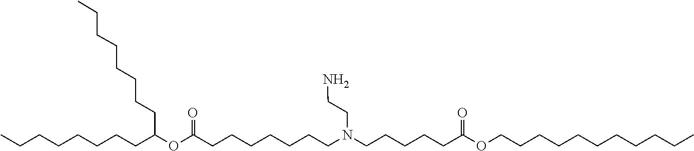 [SMILES: CCCCCCCCCCCOC(=O)CCCCCN(CCN)CCCCCCCC(=O)OC(CCCCCCCC)CCCCCCCC] | heptadecan-9-yl 8-((2- aminoethyl)(8- (nonyloxy)-8- oxooctyl)amino) octanoate |
| 2300 | 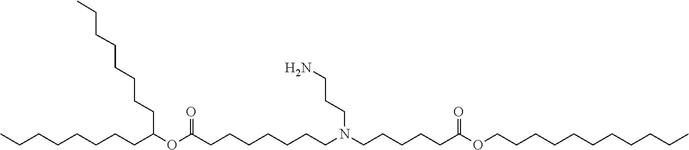 [SMILES: CCCCCCCCCCCOC(=O)CCCCCN(CCCN)CCCCCCCC(=O)OC(CCCCCCCC)CCCCCCCC] | heptadecan-9-yl 8- ((3-aminopropyl)(6- oxo-6-(undecyloxy) hexyl)amino) octanoate |
| 2301 | 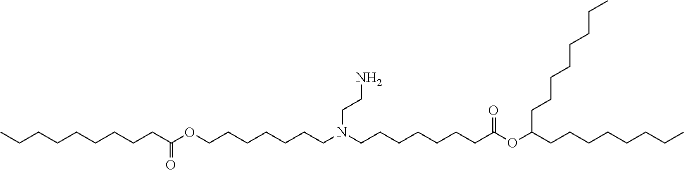 [SMILES: CCCCCCCCCC(=O)OCCCCCCCN(CCN)CCCCCCCC(=O)OC(CCCCCCCC)CCCCCCCC] | 7-((2-aminoethyl)(8- (heptadecan-9-yloxy)- 8-oxooctyl)amino) heptyl decanoate |
| 2312 | 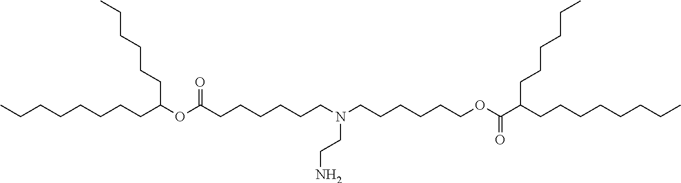 [SMILES: CCCCCCCCC(CCCCCC)OC(=O)CCCCCCN(CCN)CCCCCCOC(=O)C(CCCCCC)CCCCCCCC] | ((2-aminoethyl) azanediyl)bis (hexane-6,1-diyl) bis(2-hexyldecanoate) |
| 2313 | 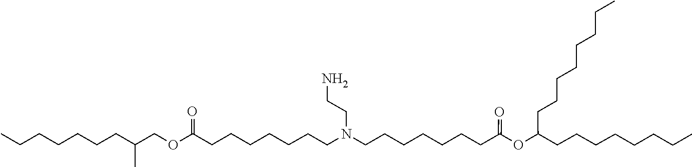 [SMILES: CCCCCCCCC(CCCCCCCC)OC(=O)CCCCCCCN(CCN)CCCCCCCC(=O)OCC(C)CCCCCCC] | heptadecan-9-yl 8-((2- aminoethyl)(8-((2- methylnonyl)oxy)-8- oxooctyl)amino) octanoate |
| 2314 | 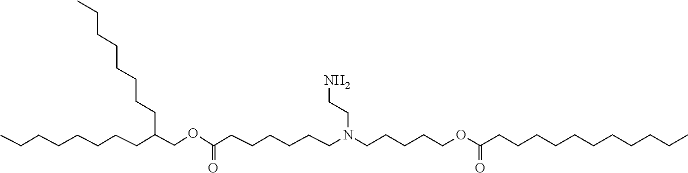 [SMILES: CCCCCCCCCCCC(=O)OCCCCCN(CCN)CCCCCCC(=O)OCC(CCCCCCCC)CCCCCCCC] | 5-((2-aminoethyl)(7- ((2-octyldecanoyl) oxy)heptyl)amino) pentyl dodecanoate |
| | 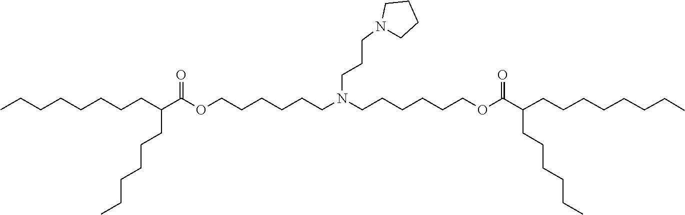 [SMILES: CCCCCCCCC(CCCCCC)C(=O)OCCCCCCN(CCCCCCOC(=O)C(CCCCCC)CCCCCCCC)CCCN1CCCC1] | ((3-(pyrrolidin-1- yl)propyl)azanediyl) bis(hexane-6,1-diyl) bis(2-hexyldecanoate) |
| | 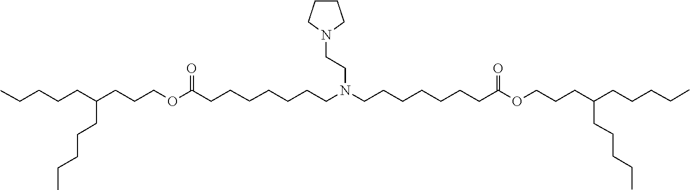 [SMILES: CCCCCC(CCCCC)CCCOC(=O)CCCCCCCN(CCCCCCCC(=O)OCCCC(CCCCC)CCCCC)CCN1CCCC1] | bis(4-pentylnonyl) 8,8′-((2-(pyrrolidin-1- yl)ethyl)azanediyl) dioctanoate |
| | 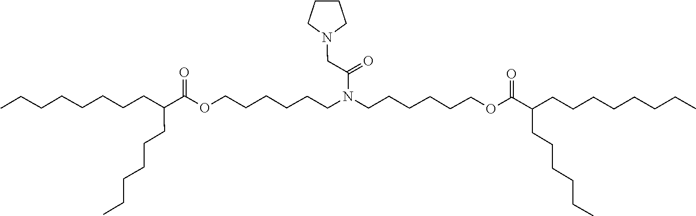 [SMILES: CCCCCCCCC(CCCCCC)C(=O)OCCCCCCN(CCCCCCOC(=O)C(CCCCCC)CCCCCCCC)C(=O)CN1CCCC1] | ((2-(pyrrolidin-1- yl)acetyl)azanediyl) bis(hexane-6,1-diyl) bis(2-hexyldecanoate) |
| | 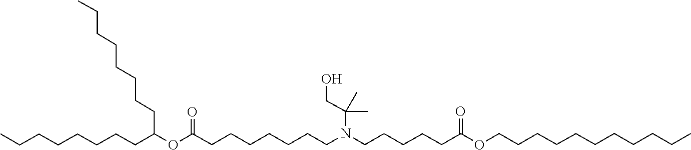 [SMILES: CCCCCCCCCCCOC(=O)CCCCCN(CCCCCCCC(=O)OC(CCCCCCCC)CCCCCCCC)C(C)(C)CO] | heptadecan-9-yl 8-((1- hydroxy-2-methyl- propan-2-yl)(6-oxo- 6-(undecyloxy)hexyl) amino)octanoate |
| | 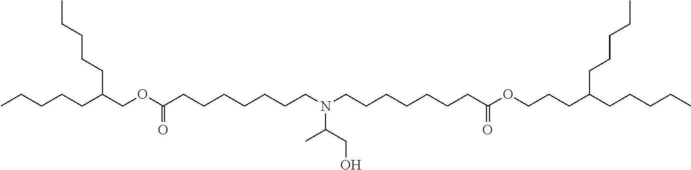 [SMILES: CCCCCC(CCCCC)CCCOC(=O)CCCCCCCN(CCCCCCCC(=O)OCC(CCCCC)CCCCC)C(C)CO] | bis(4-pentylnonyl) 8,8′-((1-hydroxy- propan-2-yl) azanediyl)dioctanoate |
| | 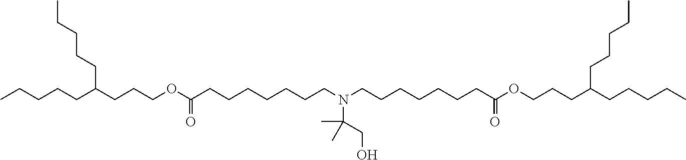 [SMILES: CCCCCC(CCCCC)CCCOC(=O)CCCCCCCN(CCCCCCCC(=O)OCCCC(CCCCC)CCCCC)C(C)(C)CO] | bis(4-pentylnonyl) 8,8′-((1-hydroxy-2- methylpropan-2- yl)azanediyl) dioctanoate |
| | 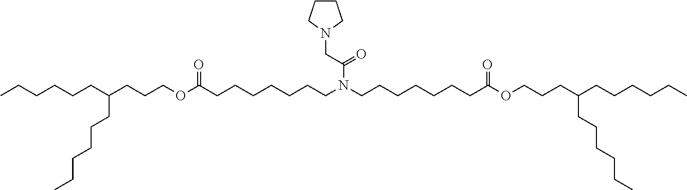 [SMILES: CCCCCCC(CCCCCC)CCCOC(=O)CCCCCCCN(CCCCCCCC(=O)OCCCC(CCCCCC)CCCCCC)C(=O)CN1CCCC1] | bis(4-hexyldecyl) 8,8′- ((2-(pyrrolidin-1-yl) acetyl)azanediyl) dioctanoate |
| | 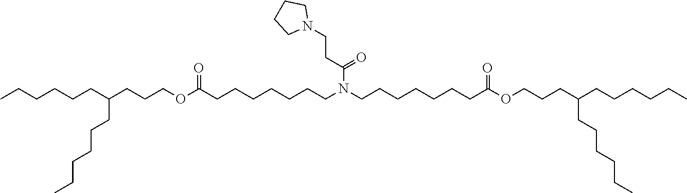 [SMILES: CCCCCCC(CCCCCC)CCCOC(=O)CCCCCCCN(CCCCCCCC(=O)OCCCC(CCCCCC)CCCCCC)C(=O)CCN1CCCC1] | bis(4-hexyldecyl) 8,8′-((3-(pyrrolidin- 1-yl)propanoyl) azanediyl)dioctanoate |
| | 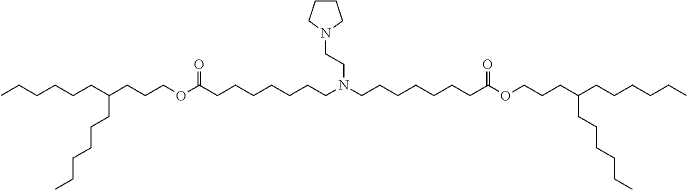 [SMILES: CCCCCCC(CCCCCC)CCCOC(=O)CCCCCCCN(CCCCCCCC(=O)OCCCC(CCCCCC)CCCCCC)CCN1CCCC1] | bis(4-hexyldecyl) 8,8′- ((2-(pyrrolidin-1-yl) ethyl)azanediyl) dioctanoate |
| | 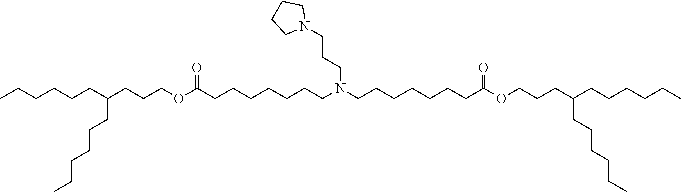 [SMILES: CCCCCCC(CCCCCC)CCCOC(=O)CCCCCCCN(CCCCCCCC(=O)OCCCC(CCCCCC)CCCCCC)CCCN1CCCC1] | bis(4-hexyldecyl) 8,8′-((3-(pyrrolidin-1- yl)propyl)azanediyl) dioctanoate |
| | 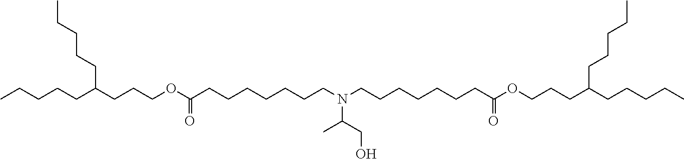 [SMILES: CCCCCC(CCCCC)CCCOC(=O)CCCCCCCN(CCCCCCCC(=O)OCCCC(CCCCC)CCCCC)C(C)CO] | bis(4-hexyldecyl) 8,8′-((1-hydroxy- propan-2-yl) azanediyl)dioctanoate |
| | 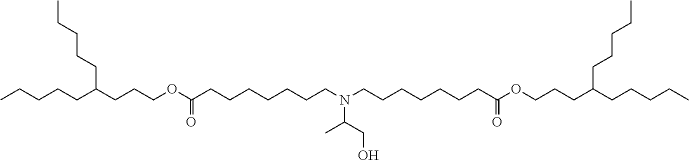 [SMILES: CCCCCC(CCCCC)CCCOC(=O)CCCCCCCN(CCCCCCCC(=O)OCCCC(CCCCC)CCCCC)C(C)CO] | bis(4-hexyldecyl) 8,8′-((1-hydroxy-2- methylpropan-2-yl) azanediyl)dioctanoate |
| | 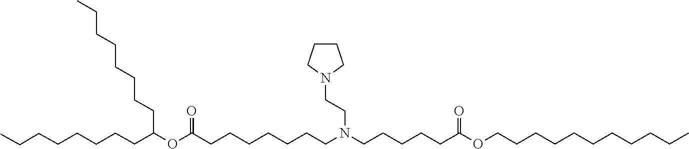 [SMILES: CCCCCCCCCCCOC(=O)CCCCCN(CCCCCCCC(=O)OC(CCCCCCCC)CCCCCCCC)CCN1CCCC1] | heptadecan-9-yl 8-((6- oxo-6-(undecyloxy) hexyl)(2-(pyrrolidin- 1-yl)ethyl)amino) octanoate |
| | 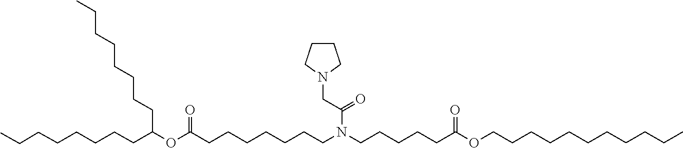 [SMILES: CCCCCCCCCCCOC(=O)CCCCCN(CCCCCCCC(=O)OC(CCCCCCCC)CCCCCCCC)C(=O)CN1CCCC1] | heptadecan-9-yl 8-(N- (6-oxo-6- (undecyloxy)hexyl)-2- (pyrrolidin-1-yl) acetamido)octanoate |
| | 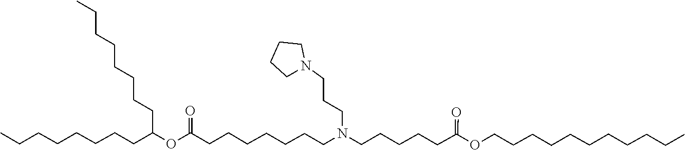 [SMILES: CCCCCCCCCCCOC(=O)CCCCCN(CCCCCCCC(=O)OC(CCCCCCCC)CCCCCCCC)CCCN1CCCC1] | heptadecan-9-yl 8-((6- oxo-6-(undecyloxy) hexyl)(3-(pyrrolidin- 1-yl)propyl)amino) octanoate |
| | 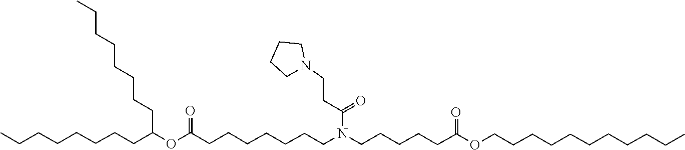 [SMILES: CCCCCCCCCCCOC(=O)CCCCCN(CCCCCCCC(=O)OC(CCCCCCCC)CCCCCCCC)C(=O)CCN1CCCC1] | heptadecan-9-yl 8- (N-(6-oxo-6- (undecyloxy)hexyl)- 3-(pyrrolidin-1-yl) propanamido) octanoate |
| | 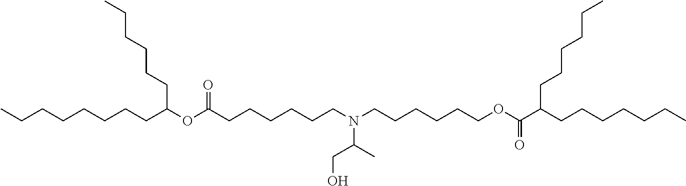 [SMILES: CCCCCCCCC(CCCCCC)OC(=O)CCCCCCN(CCCCCCOC(=O)C(CCCCCC)CCCCCCC)C(C)CO] | ((1-hydroxypropan- 2-yl)azanediyl)bis (hexane-6,1-diyl) bis(2-hexyldecanoate) |
| | 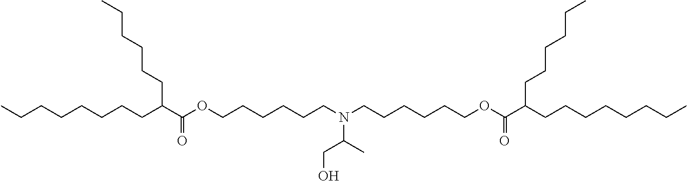 [SMILES: CCCCCCCCC(CCCCCC)C(=O)OCCCCCCN(CCCCCCOC(=O)C(CCCCCC)CCCCCCCC)C(C)CO] | ((1-hydroxy-2- methylpropan-2- yl)azanediyl)bis (hexane-6,1-diyl) bis(2-hexyldecanoate) |
| | 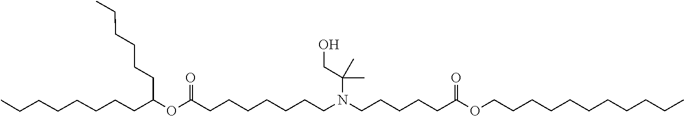 [SMILES: CCCCCCCCCCCOC(=O)CCCCCN(CCCCCCCC(=O)OC(CCCCCC)CCCCCCCC)C(C)(C)CO] | pentadecan-7-yl 8-((1- hydroxy-2-methyl- propan-2-yl)(6-oxo- 6-(undecyloxy)hexyl) amino)octanoate |
| | 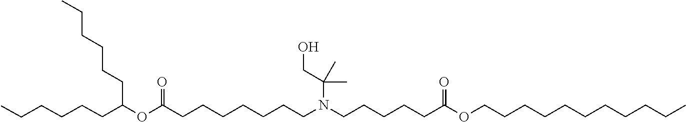 [SMILES: CCCCCCCCCCCOC(=O)CCCCCN(CCCCCCCC(=O)OC(CCCCCC)CCCCCC)C(C)(C)CO] | tridecan-7-yl 8-((1- hydroxy-2-methyl- propan-2-yl)(6-oxo- 6-(undecyloxy) hexyl)amino) octanoate |
| | 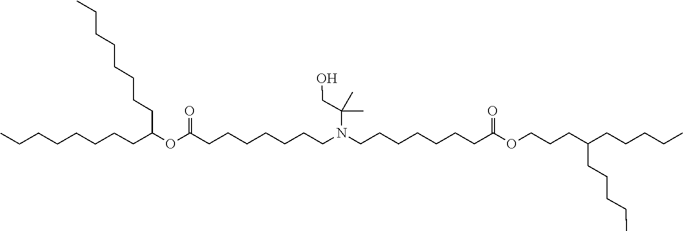 [SMILES: CCCCCCCCC(CCCCCCCC)OC(=O)CCCCCCCN(CCCCCCCC(=O)OCCCC(CCCCC)CCCCC)C(C)(C)CO] | heptadecan-9-yl 8- ((1-hydroxy-2- methylpropan-2-yl) (8-oxo-8-((4- pentylnonyl)oxy) octyl)amino)octanoate |
| | 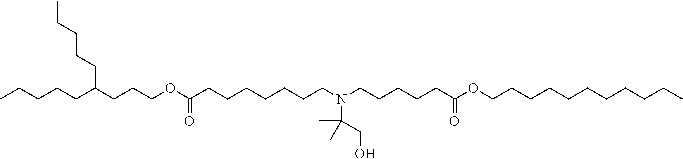 [SMILES: CCCCCCCCCCCOC(=O)CCCCCN(CCCCCCCC(=O)OCCCC(CCCCC)CCCCC)C(C)(C)CO] | 4-pentylnonyl 8-((1- hydroxy-2-methyl- propan-2-yl)(6-oxo- 6-(undecyloxy)hexyl) amino)octanoate |
| | 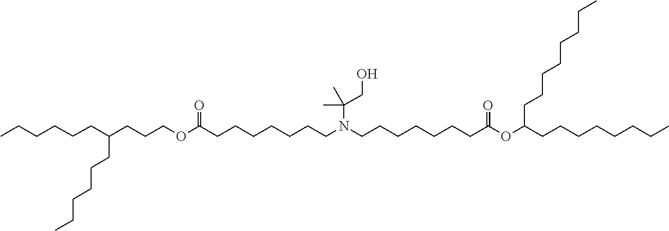 [SMILES: CCCCCCCCC(CCCCCCCC)OC(=O)CCCCCCCN(CCCCCCCC(=O)OCCCC(CCCCCC)CCCCCC)C(C)(C)CO] | heptadecan-9-yl 8- ((8-((4-hexyldecyl) oxy)-8-oxooctyl)(1- hydroxy-2-methyl- propan-2-yl)amino) octanoate |
| | 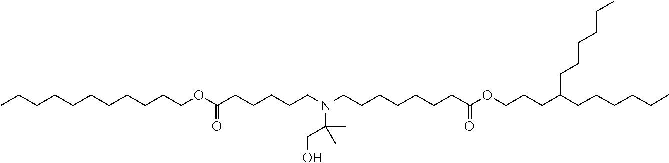 [SMILES: CCCCCCCCCCCOC(=O)CCCCCN(CCCCCCCC(=O)OCCCC(CCCCCC)CCCCCC)C(C)(C)CO] | 4-hexyldecyl 8-((1- hydroxy-2-methyl- propan-2-yl)(6-oxo-6- (undecyloxy)hexyl) amino)octanoate |
| | 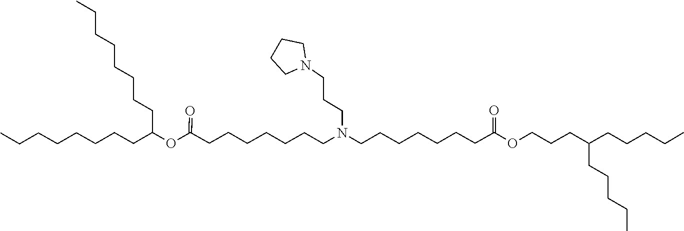 [SMILES: CCCCCCCCC(CCCCCCCC)OC(=O)CCCCCCCN(CCCCCCCC(=O)OCCCC(CCCCC)CCCCC)CCCN1CCCC1] | heptadecan-9-yl 8-((8- oxo-8-((4- pentylnonyl)oxy) octyl)(3-(pyrrolidin- 1-yl)propyl)amino) octanoate |
| | 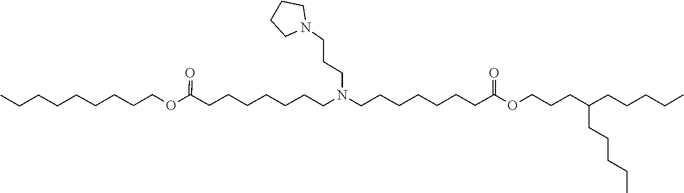 [SMILES: CCCCCCCCCOC(=O)CCCCCCCN(CCCCCCCC(=O)OCCCC(CCCCC)CCCCC)CCCN1CCCC1] | 4-pentylnonyl 8-((6- oxo-6-(undecyloxy) hexyl)(3-(pyrrolidin- 1-yl)propyl)amino) octanoate |
| | 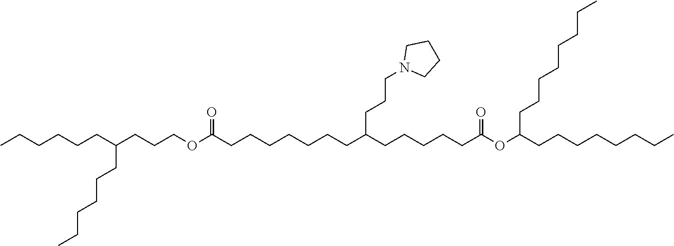 [SMILES: CCCCCCCCC(CCCCCCCC)OC(=O)CCCCCC(CCCCCCCC(=O)OCCCC(CCCCCC)CCCCCC)CCCN1CCCC1] | heptadecan-9-yl 8-((8- ((4-hexyldecyl)oxy)- 8-oxooctyl)(3- (pyrrolidin-1-yl) propyl)amino) octanoate |
| | 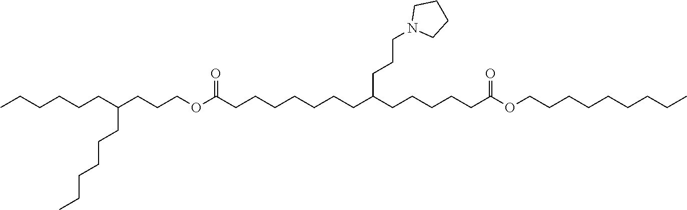 [SMILES: CCCCCCCCCOC(=O)CCCCCC(CCCCCCCC(=O)OCCCC(CCCCCC)CCCCCC)CCCN1CCCC1] | 4-hexyldecyl 8-((6- oxo-6-(undecyloxy) hexyl)(3-(pyrrolidin- 1-yl)propyl)amino) octanoate |
| | 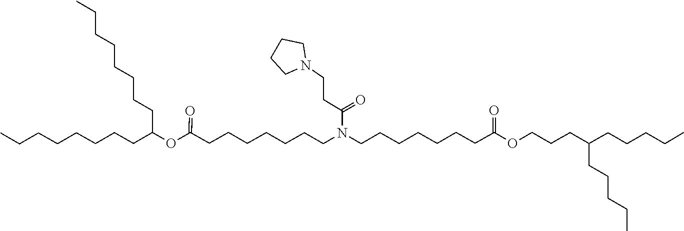 [SMILES: CCCCCCCCC(CCCCCCCC)OC(=O)CCCCCCCN(CCCCCCCC(=O)OCCCC(CCCCC)CCCCC)C(=O)CCN1CCCC1] | heptadecan-9-yl 8- (N-(8-oxo-8-((4- pentylnonyl)oxy) octyl)-3-(pyrrolidin-1- yl)propanamido) octanoate |
| | 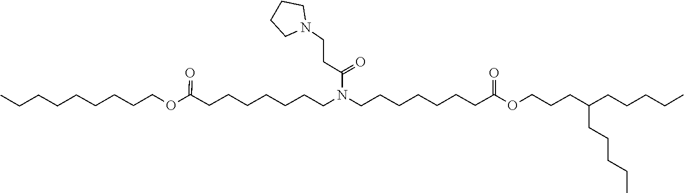 [SMILES: CCCCCCCCCOC(=O)CCCCCCCN(CCCCCCCC(=O)OCCCC(CCCCC)CCCCC)C(=O)CCN1CCCC1] | 4-pentylnonyl 8-(N- (6-oxo-6- (undecyloxy)hexyl)- 3-(pyrrolidin-1-yl) propanamido) octanoate |
| | 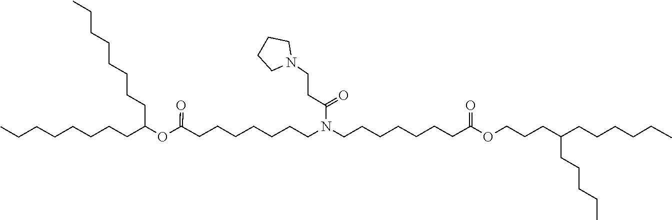 [SMILES: CCCCCCCCC(CCCCCCCC)OC(=O)CCCCCCCN(CCCCCCCC(=O)OCCCC(CCCCC)CCCCCC)C(=O)CCN1CCCC1] | heptadecan-9-yl 8- (N-(8-((4-hexyldecyl) oxy)-8-oxooctyl)-3- (pyrrolidin-1-yl) propanamido) octanoate |
| | 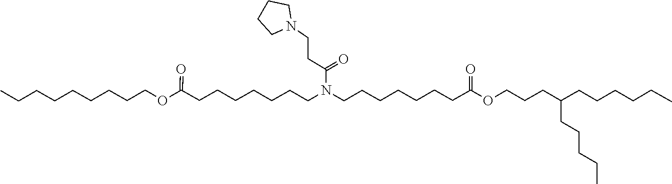 [SMILES: CCCCCCCCCOC(=O)CCCCCCCN(CCCCCCCC(=O)OCCCC(CCCCC)CCCCCC)C(=O)CCN1CCCC1] | 4-hexyldecyl 8-(N- (6-oxo-6- (undecyloxy)hexyl)- 3-(pyrrolidin-1- yl)propanamido) octanoate |
| | 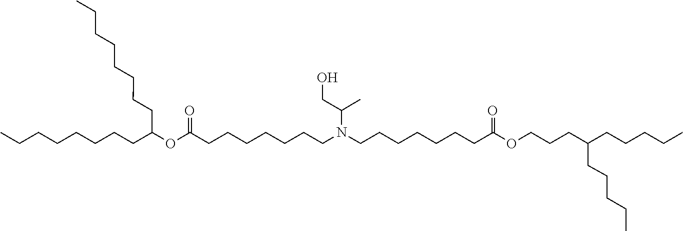 [SMILES: CCCCCCCCC(CCCCCCCC)OC(=O)CCCCCCCN(CCCCCCCC(=O)OCCCC(CCCCC)CCCCC)C(C)CO] | heptadecan-9-yl 8- ((1-hydroxypropan- 2-yl)(8-oxo-8-((4- pentylnonyl)oxy) octyl)amino) octanoate |
| | 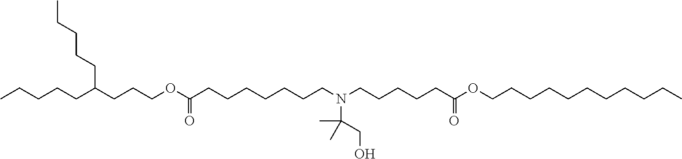 [SMILES: CCCCCCCCCCCOC(=O)CCCCCN(CCCCCCCC(=O)OCCCC(CCCCC)CCCCC)C(C)(C)CO] | 4-pentylnonyl 8-((1- hydroxypropan-2-yl) (6-oxo-6- (undecyloxy)hexyl) amino)octanoate |
| | 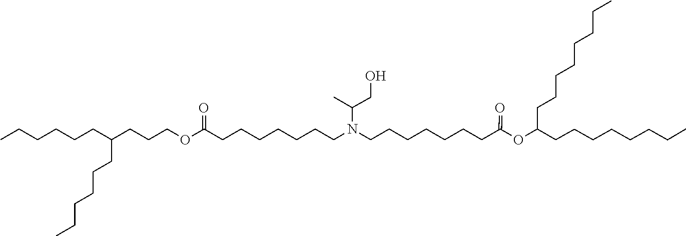 [SMILES: CCCCCCCCC(CCCCCCCC)OC(=O)CCCCCCCN(CCCCCCCC(=O)OCCCC(CCCCCC)CCCCCC)C(C)CO] | heptadecan-9-yl 8- ((8-((4-hexyldecyl) oxy)-8-oxooctyl)(1- hydroxypropan-2- yl)amino)octanoate |
| | 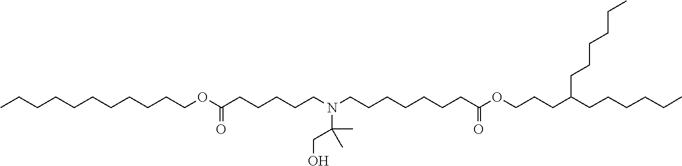 [SMILES: CCCCCCCCCCCOC(=O)CCCCCN(CCCCCCCC(=O)OCCCC(CCCCCC)CCCCCC)C(C)(C)CO] | 4-hexyldecyl 8-((1- hydroxypropan-2- yl)(6-oxo-6- (undecyloxy)hexyl) amino)octanoate |
| | 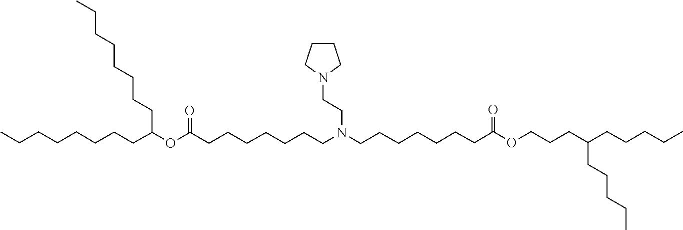 [SMILES: CCCCCCCCC(CCCCCCCC)OC(=O)CCCCCCCN(CCCCCCCC(=O)OCCCC(CCCCC)CCCCC)CCN1CCCC1] | heptadecan-9-yl 8- ((8-oxo-8-((4- pentylnonyl)oxy) octyl)(2-(pyrrolidin- 1-yl)ethyl)amino) octanoate |
| | 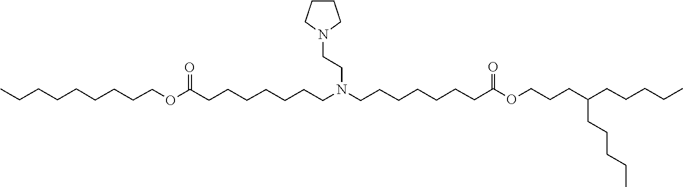 [SMILES: CCCCCCCCCOC(=O)CCCCCCCN(CCCCCCCC(=O)OCCCC(CCCCC)CCCCC)CCN1CCCC1] | 4-pentylnonyl 8-((6- oxo-6-(undecyloxy) hexyl)(2-(pyrrolidin- 1-yl)ethyl)amino) octanoate |
| | 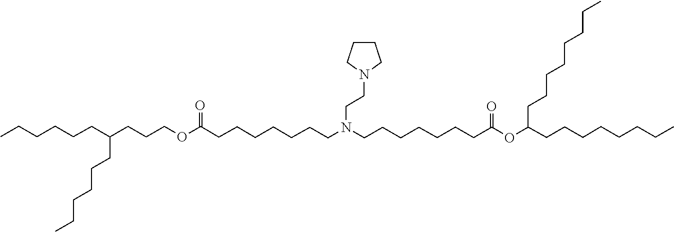 [SMILES: CCCCCCCCC(CCCCCCCC)OC(=O)CCCCCCCN(CCCCCCCC(=O)OCCCC(CCCCCC)CCCCCC)CCN1CCCC1] | heptadecan-9-yl 8- ((8-((4-hexyldecyl) oxy)-8-oxooctyl)(2- (pyrrolidin-1-yl) ethyl)amino) octanoate |
| | 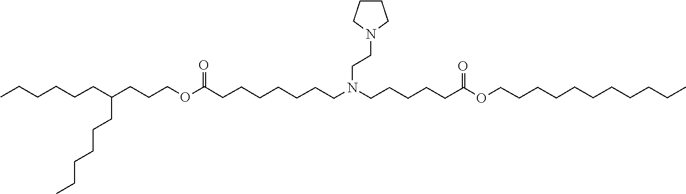 [SMILES: CCCCCCCCCCCOC(=O)CCCCCN(CCCCCCCC(=O)OCCCC(CCCCCC)CCCCCC)CCN1CCCC1] | 4-hexyldecyl 8-((6- oxo-6-(undecyloxy) hexyl)(2-(pyrrolidin- 1-yl)ethyl)amino) octanoate |
| | 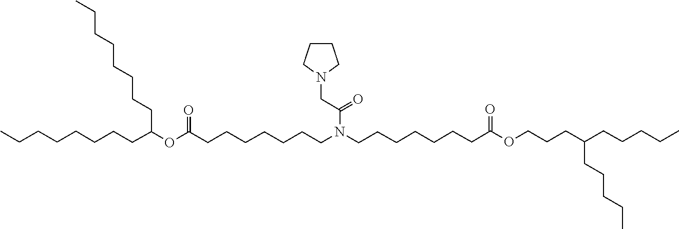 [SMILES: CCCCCCCCC(CCCCCCCC)OC(=O)CCCCCCCN(CCCCCCCC(=O)OCCCC(CCCCC)CCCCC)C(=O)CN1CCCC1] | heptadecan-9-yl 8- (N-(8-oxo-8-((4- pentylnonyl)oxy) octyl)-2-(pyrrolidin-1- yl)acetamido) octanoate |
| | 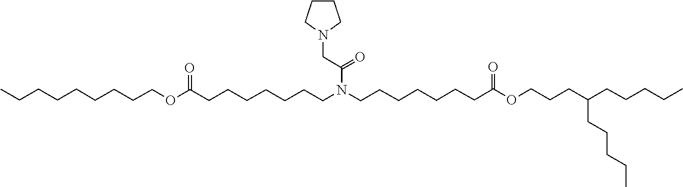 [SMILES: CCCCCCCCCOC(=O)CCCCCCCN(CCCCCCCC(=O)OCCCC(CCCCC)CCCCC)C(=O)CN1CCCC1] | 4-pentylnonyl 8-(N- (6-oxo-6- (undecyloxy)hexyl)- 2-(pyrrolidin-1-yl) acetamido)octanoate |
| | 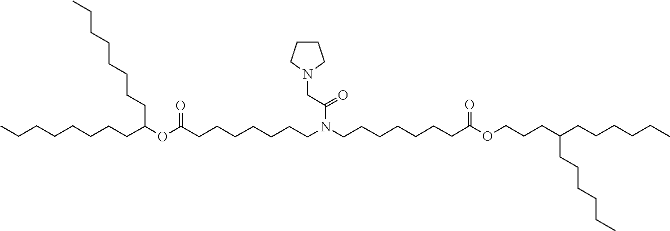 [SMILES: CCCCCCCCC(CCCCCCCC)OC(=O)CCCCCCCN(CCCCCCCC(=O)OCCCC(CCCCCC)CCCCCC)C(=O)CN1CCCC1] | heptadecan-9-yl 8- (N-(8-((4-hexyldecyl) oxy)-8-oxooctyl)-2- (pyrrolidin-1-yl) acetamido)octanoate |
| | 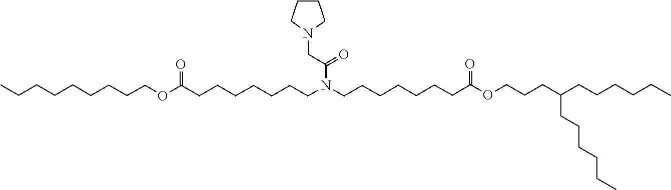 [SMILES: CCCCCCCCCOC(=O)CCCCCCCN(CCCCCCCC(=O)OCCCC(CCCCCC)CCCCCC)C(=O)CN1CCCC1] | 4-hexyldecyl 8-(N- (6-oxo-6- (undecyloxy)hexyl)- 2-(pyrrolidin-1-yl) acetamido)octanoate |

### Ionizable Lipid Compounds iv)

In some embodiments, the ionizable lipid is a lipid comprising at least one head group and at least one tail group of formula (TI) or (TI′):

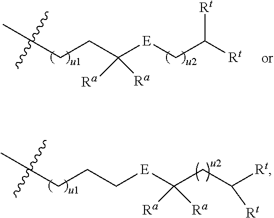

a pharmaceutically acceptable salt thereof, or a stereoisomer of any of the foregoing, wherein:
E is each independently a biodegradable group;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;

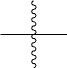

represents the bond connecting the tail group to the head group; and
wherein the lipid has a pKa from about 4 to about 8.

In some embodiments, E is —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, or —S—S—, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl; and r is 1, 2, 3, 4, or 5. In some embodiments, E is each independently —OC(O)—, —C(O)O—, —N(R₇)C(O)—, or

—C(O)N(R⁷)—, wherein R⁷ is independently H, alkyl, alkenyl, cycloalkyl, hydroxyalkyl, or aminoalkyl.

In some embodiments, the lipid comprises at least one head group and at least one tail group of formula (TII):

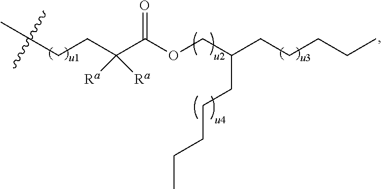

wherein u3 and u4 are each independently 0, 1, 2, 3, or 4. The definitions of other variables in (TII) are the same as those defined above in (TI).

In some embodiments, the lipid comprises at least one head group and at least one tail group of formula (TIII):

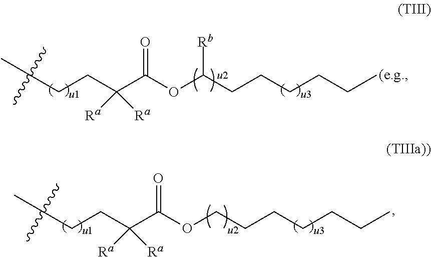

wherein u3 is 0, 1, 2, 3, 4, 5, 6, or 7; and R^{b} is in each occasion independently H or C₁-C₄ alkyl. The definitions of other variables in (TIII) are the same as those defined above in (TI).

In some embodiments, the lipid comprises at least one head group and at least one tail group of formula (TIV):

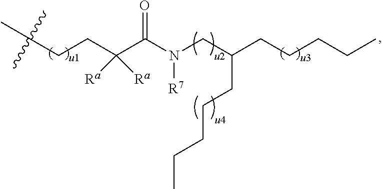

wherein u3 and u4 are each independently 0, 1, 2, 3, or 4. The definitions of other variables in (TIV) are the same as those defined above in (TI).

In some embodiments, the lipid comprises at least one head group and at least one tail group of formula (TV)

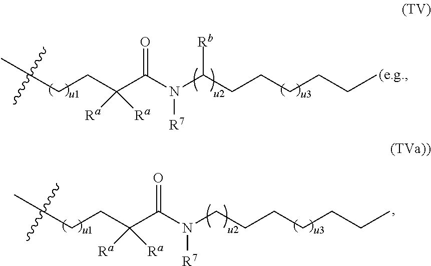

wherein u3 is 0, 1, 2, 3, 4, 5, 6, or 7; R⁷ is each independently H or methyl; and R^{b} is in each occasion independently H or C₁-C₄ alkyl. The definitions of other variables in (TV) are the same as those defined above in (TI).

In some embodiments, the lipid comprises at least one head group and at least one tail group of formula (TII′)

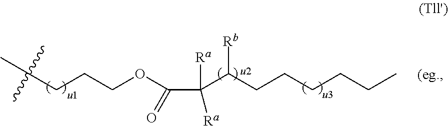

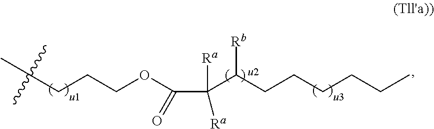

wherein u3 is 0, 1, 2, 3, 4, 5, 6, or 7; and R^{b} is in each occasion independently H or C₁-C₄ alkyl. The definitions of other variables in (TII′) are the same as those defined above in (TI′).

In some embodiments, the lipid comprises at least one head group and at least one tail group of formula (TII′)

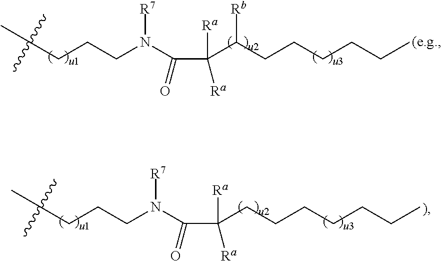

wherein u3 is 0, 1, 2, 3, 4, 5, 6, or 7; R⁷ is each independently H or methyl; and R^{b} is in each occasion independently H or C₁-C₄ alkyl. The definitions of other variables in (TIII′) are the same as those defined above in (TI′).

In some embodiments, the lipid comprises at least one tail group of the formulas (TII), (TIII), (TIV), (TV), (TII′), and (TIII′), wherein
R⁷ is each independently H or methyl;
R^{b} is in each occasion independently H or C₁-C₄ alkyl;
u1 and u2 are each independently 0, 1, 2, 3, 4, 5, 6, or 7;
u3 and u4 are each independently 0, 1, 2, 3, 4, 5, 6, or 7; and
wherein the lipid has a pKa from about 4 to about 8.

In some embodiments, the lipid comprises two, three, four, o or more tail groups that have a formula of (T), (TI), (TII), (TIII), (TIV), (TV), (TII′), and/or (TIII′), and each tail group may be the same or different.

In some embodiments, in any of the above formulas (T), (TI), (TII), and (TIII), (TIV), (TV), (TI′), (TII′), and/or (TIII′), R^{a} is each independently C₁-C₅ branched or unbranched alkyl, C₂-C₅ branched or unbranched alkenyl, or C₂-C₅ branched or unbranched alkynyl. in some embodiments, R^{a} is each independently C₁-C₃ branched or unbranched alkyl. in one embodiment, each R^{a} is methyl.

In some embodiments, in any of the above formulas (T), (TI), (TII), and (TIII), (TIV), (TV), (TI′), (TII′), and/or (TIII′), u1 is 3, 4, or 5. in some embodiments, in any of the above formulas (T), (TI), (TII), and (TIII), (TIV), (TV), (TI′), (TII′), and/or (TIII′), u2 is 0, 1, 2, or 3. in some embodiments, in any of the above formulas (T), (TI), (TII), and (TIII), (TIV), (TV), (TI′), (TII′), and/or (TIII′), u3 and u4 are each independently 1-7, for instance, u3 and u4 are each independently 1, 2, 3, or 4.

In some embodiments, the lipid comprises at least one tail of formula (TIII), wherein each R^{a} is methyl; R^{b} is in each occasion independently H, ethyl, or butyl; u1 is 3-5, u2 is 0-3, and u3 is 1-7 (e.g., 1-4). In some embodiments, the lipid comprises at least one two tails of formula (TIII), wherein the two tails of formula (TIII) are the same or different. in some embodiments, the lipid comprises at least three tails of formula (TIII), wherein each tail may be the same or different. in some embodiments, the lipid has four tails of formula (TIII), wherein each tail may be the same or different. In some embodiments, in each tail of formula (TIII), each R^{a} is methyl, and u1 is 3, u2 is 2, and u3 is 4.

In some embodiments, the lipid comprises at least one tail of formula (TII), wherein each R^{a} is methyl, u1 is 3-5, u2 is 0-3, u3 is 1-4, and u4 is 1-4. In some embodiments, the lipid has at least two tails of formula (TII), wherein the two tails of formula (TII) are the same. In some embodiments, the lipid has at least two tails of formula (TII), wherein the two tails of formula (TII) are or different. In some embodiments, the lipid comprises at least three tails of formula (TII), wherein each tail may be the same or different. In some embodiments, the lipid has four tails of formula (TII), wherein each tail may be the same or different.

In some embodiments, the lipid comprises at least one tail of formula (TIV), wherein each R^{a} is methyl, u1 is 3-5, u2 is 0-3, u3 is 1-4, and u4 is 1-4. In some embodiments, the lipid comprises at least two tails of formula (TIV), wherein each tail may be the same or different. In some embodiments, the lipid comprises at least three tails of formula (TIV), wherein each tail may be the same or different. In some embodiments, the lipid comprises at least four tails of formula (TIV), wherein each tail may be the same or different.

In some embodiments, the lipid comprises at least two tails of formula (TV), wherein each tail may be the same or different. In some embodiments, the lipid comprises at least three tails of formula (TV), wherein each tail may be the same or different. In some embodiments, the lipid comprises at least four tails of formula (TV), wherein each tail may be the same or different.

In some embodiments, the lipid has at least two tails of formula (TII′), wherein each tail may be the same or different. In some embodiments, the lipid has at least three tails of formula (TII′), wherein each tail may be the same or different. In some embodiments, the lipid has at least four tails of formula (TII′), wherein each tail may be the same or different.

In some embodiments, the lipid has at least two tails of formula (TIII′), wherein each tail may be the same or different. In some embodiments, the lipid has at least three tails of formula (TIII′), wherein each tail may be the same or different. In some embodiments, the lipid has at least four tails of formula (TIII′), wherein each tail may be the same or different.

In some embodiments, the lipid has at least one tail of formula (TII) and/or at least one tail of formula (TIII); the lipid further comprises at least one tail that does not have a formula (T), (TI), (TII), (TIII), (TIV), (TV), (TII′), and/or (TIII′). That is to say, the lipid further comprises at least one tail that does not contain a gem-di functional groups bonded to the same carbon next to E (e.g., —C(O)O—).

In some embodiments, the lipid further comprises at least one tail that does not have a formula (T), (TI), (TII), (TIII), (TIV), (TV), (TI′), (TII′), and/or (TIII′). That is to say, the lipid further comprises at least one tail that does not contain a gem-di functional groups bonded to the same carbon next to E.

In some embodiments, the lipid further comprises at least one tail that does not have a formula (T), (TI), (TII), (TIII), (TIV), (TV), (TI′), (TII′), and/or (TIII′). That is to say, the lipid further comprises at least one tail that does not contain a gem-di functional groups bonded to the same carbon next to E.

In some embodiments, the lipid further comprises at least one tail of formula (TNG-1):

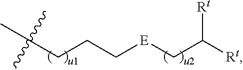

wherein
E is each independently a biodegradable group as described herein (e.g., —OC(O)—, —C(O)O—, —N(R⁷)C(O)—, —S—S—, or —C(O)N(R₇)—)
u1 and u2 are each independently 0, 1, 2, 3, 4, 5, 6, or 7; and
R⁷ is independently H, alkyl, alkenyl, cycloalkyl, hydroxyalkyl, or aminoalkyl.

In some embodiments, the at least one tail of formula (TNG-1) can be represented by

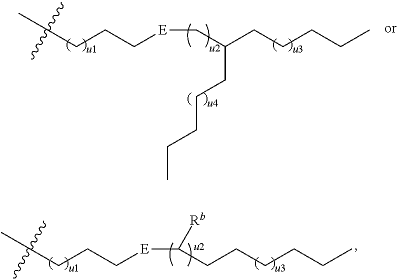

wherein
u3 and u4 are each independently 0, 1, 2, 3, 4, 5, 6, or 7; and
R^{b} is in each occasion independently H or C₁-C₄ alkyl.

All the embodiments above regarding the definitions of E, R^{b}, R^{t}, u1, u2, u3 and u4, as described above relating to the tail group containing a gem-di functional group bonded to the same carbon next to E, having a formula (T), (TI), (TII), (TIII), (TIV), (TV), (TII′), or (TIII′), are also applicable to the tail group that does not contain a gem-di functional groups bonded to the same carbon next to E, having a formula (TNG-I), (TNG-II), or (TNG-III).

In some embodiments, the lipid further comprises at least two tails that do not have a formula (T), (TI), (TII), (TIII), (TIV), (TV), (TI′), (TII′), and/or (TIII′). In some embodiments, the lipid comprises two tail groups of formula (TNG-II) or (TNG-III), and wherein each tail group may be the same or different,

In some embodiments, the lipid further comprises at least three tails that do not have a formula (T), (TI), (TII), (TIII), (TIV), (TV), (TI′), (TII′), and/or (TIII′). In some embodiments, the lipid comprises three tail groups of formula (TNG-II) or (TNG-III), and wherein each tail group may be the same or different,

In some embodiments, the head group of the lipid has a structure of formula (HA-I):

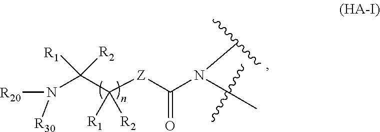

wherein:
R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
R₂₀ and R₃₀, together with the adjacent N atom, form a 3 to 7 membered heterocyclic or heteroaromatic ring containing one or more heteroatoms, optionally substituted with one or more OH, SH, halogen, alkyl, or cycloalkyl groups;
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
each of R₁₀ and R₁₁ is independently H, C₁-C₃ branched or unbranched alkyl, C₂-C₃ branched or unbranched alkenyl; or R₁₀ and R₁₁ together form a heterocyclic ring;
n is 0, 1, 2, 3 or 4;
Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, SH.

In some embodiments, R₂₀ and R₃₀ together with the adjacent N atom form a 3 to 7 membered heterocyclic or heteroaromatic ring containing one or more heteroatoms, optionally substituted with one or more OH, SH, halogen, alkyl, or cycloalkyl groups.

In some embodiments, the head group of the ionizable lipid has a structure of formula (HA-IA):

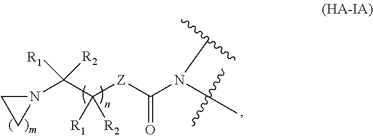

wherein:
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ are taken together to form a cyclic ring;
each of R₁₀ and R₁₁ is independently H, C₁-C₃ branched or unbranched alkyl, C₂-C₃ branched or unbranched alkenyl; or R₁₀ and R₁₁ are taken together to form a heterocyclic ring;
m is 1, 2, 3, 4, 5, 6, 7 or 8;
n is 0, 1, 2, 3 or 4;
Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, or SH; and

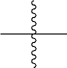

represents the bond connecting the head group to the tail group.

In some embodiments, the head group of the ionizable lipid has a structure of formula (HA-III):

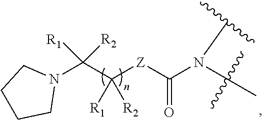

wherein Z is absent, O, S, or NR₁₂; and R₁₂ is H or C₁-C₇ branched or unbranched alkyl. The definitions of other variables in (HA-III) are the same as those defined above in (HA-IA).

In some embodiments, the head group has a structure of:

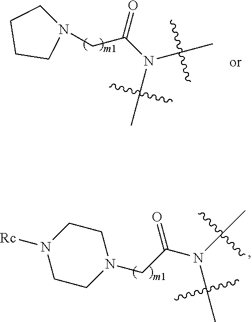

wherein:

Rc is H or alkyl, optionally substituted with OH; and
m1 is 1, 2, or 3.

In some embodiments, the head group of the ionizable lipid has a structure of formula (HA-V):

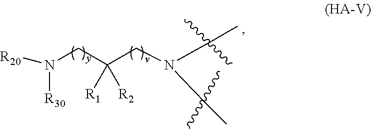

wherein:
R₁ is H, C₁-C₃ alkyl, OH, halogen, SH, or NR₁₀R₁₁;
R₂ is OH, halogen, SH, or NR₁₀R₁₁; or R₁ and R₂ can be taken together to form a cyclic ring;
R₁₀ and R₁₁ are each independently H or C₁-C₃ alkyl; or R₁₀ and R₁₁ can be taken together to form a heterocyclic ring;
R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
each of v and y is independently 1, 2, 3, or 4.

In some embodiments, the head group of the ionizable lipid has a structure of formula (HA-VI):

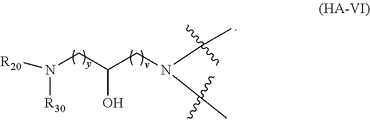

The definitions of all variables in (HA-VI) are the same as those defined above in (HA-V).

In some embodiments, in any of the above formulas (HA-V) or (HA-VI), each R₂₀ and R₃₀ are independently C₁-C₃ alkyl. In one embodiment, each R₂₀ and R₃₀ are independently methyl.

In some embodiments, the head group of the ionizable lipid has a structure of formula (HB—I):

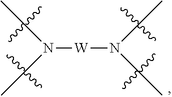

wherein W is

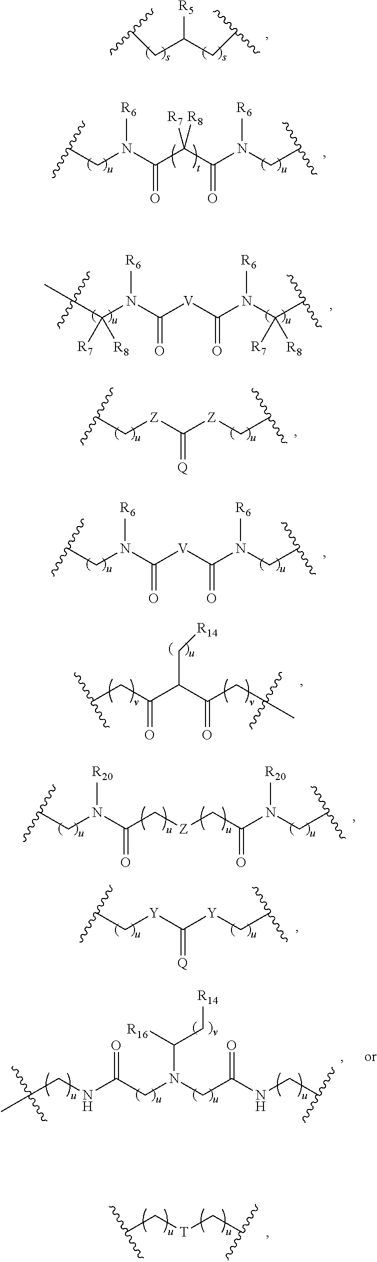

wherein
R₅ is OH, SH, (CH₂)ₛOH, or NR₁₀R₁₁;
each R₆ is independently H, C₁-C₃ branched or unbranched alkyl, C₂-C₃ branched or unbranched alkenyl, or cycloalkyl;
each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
each R₂₀ is independently H, or C₁-C₃ branched or unbranched alkyl;
R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
R₁₆ is H, ═O, ═S, or CN;
each of s, u, and t is independently 1, 2, 3, 4, or 5;
each v is independently 0, 1, 2, 3, 4, or 5;
each Y is a divalent heterocyclic;
each Z is independently absent, O, S, or NR₁₂, wherein R₁₂ is H, C₁-C₇ branched or unbranched alkyl, or C₂-C₇ branched or unbranched alkenyl;
Q is O, S, CH₂, or NR₁₃, wherein each R₁₃ is H, or C₁-C₅ alkyl;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
T is —NHC(O)O—, —OC(O)NH—, or a divalent heterocyclic.

In some embodiments, in formula (HB—I), W is

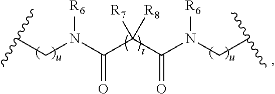

wherein:
each R₆, R₇, and R₈ are independently H or methyl; and
each of u and t is independently 1, 2, or 3.

In some embodiments, in formula (HB—I), W is

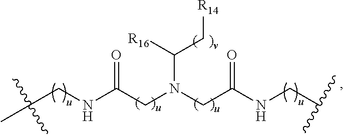

wherein:
R₁₆ is H or ═O;
R₁₄ is a nitrogen-containing 5- or 6-membered heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H or C₁-C₃ alkyl; and
each of u and v is independently 1, 2, or 3.

In some embodiments, in formula (HB—I), W is

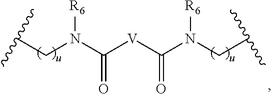

wherein:
each R₆ is independently H or methyl;
each u is independently 1, 2, or 3; and
V is C₂-C₆ alkylene or C₂-C₆ alkenylene.

In some embodiments, in formula (HB—I), W is

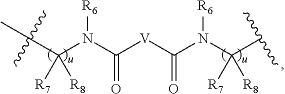

wherein:
each R₆ is independently H or methyl;
each R₇ is independently H;
each R₈ is methyl;
each u is independently 1, 2, or 3; and
V is C₂-C₆ alkylene or C₂-C₆ alkenylene.

In some embodiments, in formula (HB—I), W is

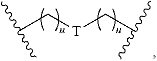

wherein:
each u is independently 1, 2, or 3; and
T is a divalent nitrogen-containing 5- or 6-membered heterocyclic.

In some embodiments, in formula (HB—I), W is

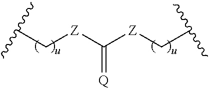

wherein:
each u is independently 1, 2, or 3;
Q is O;
each Z is independently NR₁₂, and
R₁₂ is H or C₁-C₃ alkyl.

In some embodiments, the head group has the structure of:

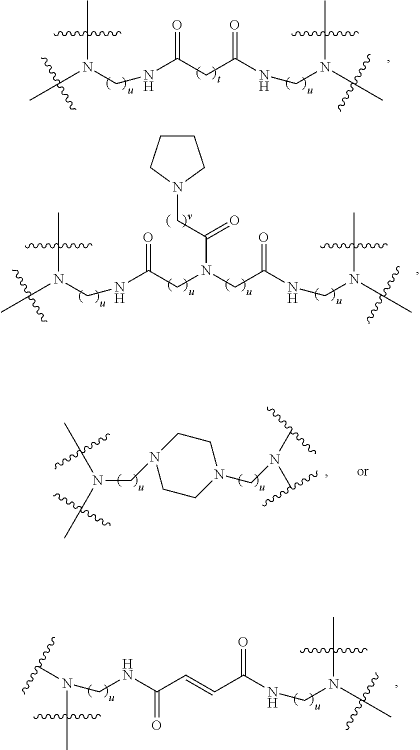

wherein each of u and t is independently 1 or 2.

In some embodiments, the head group of the ionizable lipid has a structure of formula (HC—I):

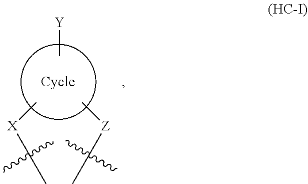

wherein

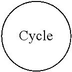

is cyclic or heterocyclic moiety;
Y is alkyl, hydroxy, hydroxyalkyl,

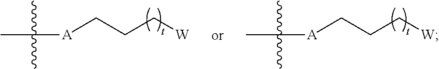

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t is 0, 1, 2, or 3;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl.

In some embodiments, the head group has a structure of formula

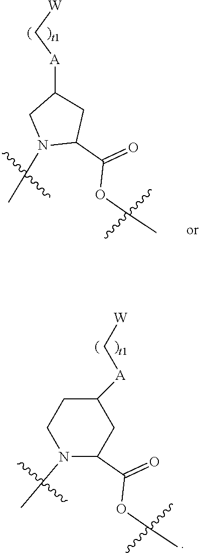

In some embodiments, in the above formulas,
A is absent, —O—, —N(R⁷)—, —OC(O)—, or —C(O)O—;
X is absent, —O—, or —C(O)—; and
Z is —O—, —C(O)O—, or —OC(O)—.

In some embodiments, the head group has a structure of formula

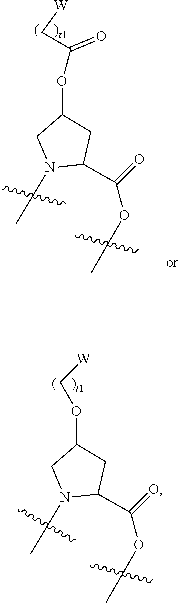

wherein t1 is 0, 1, 2, or 3.

In some embodiments, W is hydroxyl, substituted or unsubstituted hydroxyalkyl, or one of the following moieties:

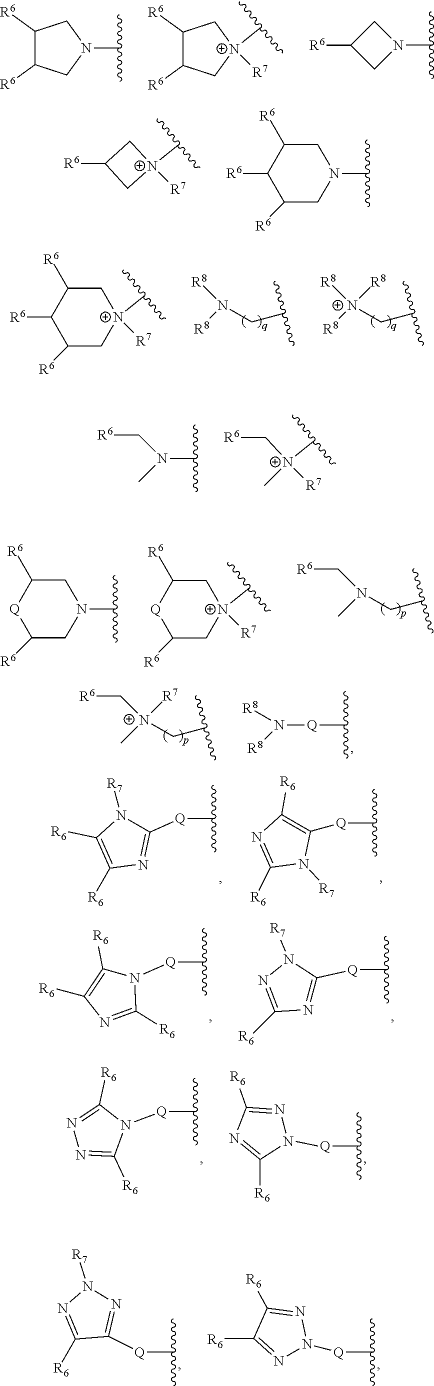

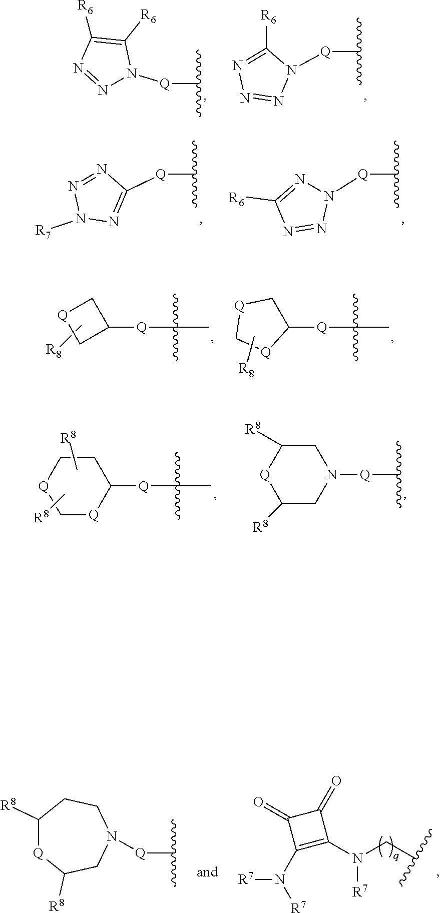

wherein
each Q is independently absent, —O—, —C(O)—, —C(S)—, —C(O)O—, —(CH₂)_{q}—C(R⁷)₂, —C(O)N(R⁷)—, —C(S)N(R⁷)—, or —N(R⁷);
R⁶ is independently H, alkyl, hydroxyl, hydroxyalkyl, alkoxy, —O-alkylene-O-alkyl, —O— alkylene-N(R⁷)₂, amino, alkylamino, aminoalkyl, thiol, thiolalkyl, or N⁺(R⁷)₃-alkylene-Q-;
each R⁸ is independently H, alkyl, hydroxyalkyl, amino, aminoalkyl, alkylamino, thiol, or thiolalkyl, heterocyclyl, heteroaryl, or two R⁸ together with the nitrogen atom may form a ring, optionally substituted with one or more alkyl, hydroxy, hydroxyalkyl, alkoxy, alkylaminoalkyl, alkylamino, aminoalkyl;
q is 0, 1, 2, 3, 4, or 5; and
p is 0, 1, 2, 3, 4, or 5.

In some embodiments, W is one of the following: OH,

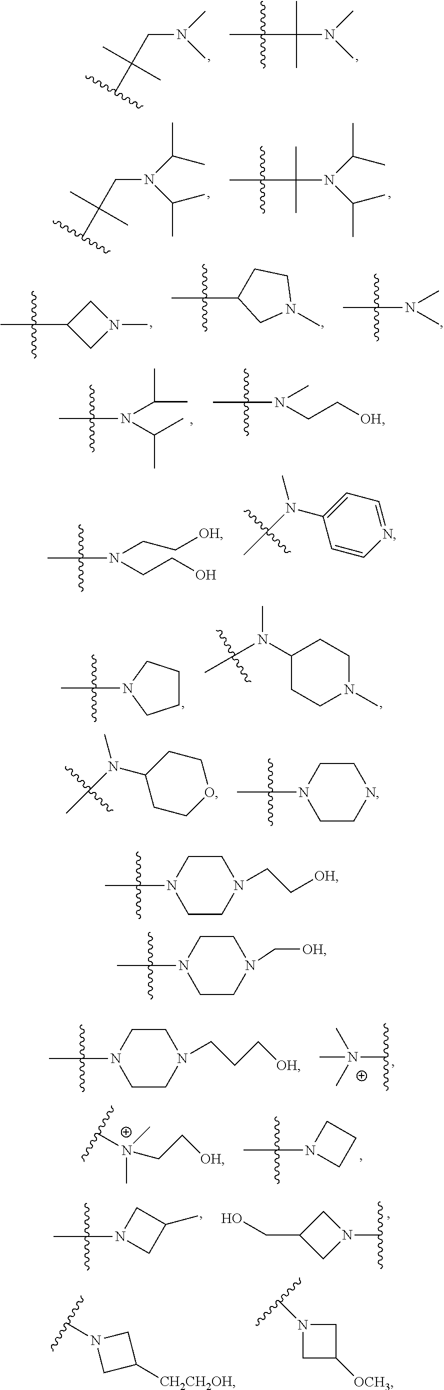

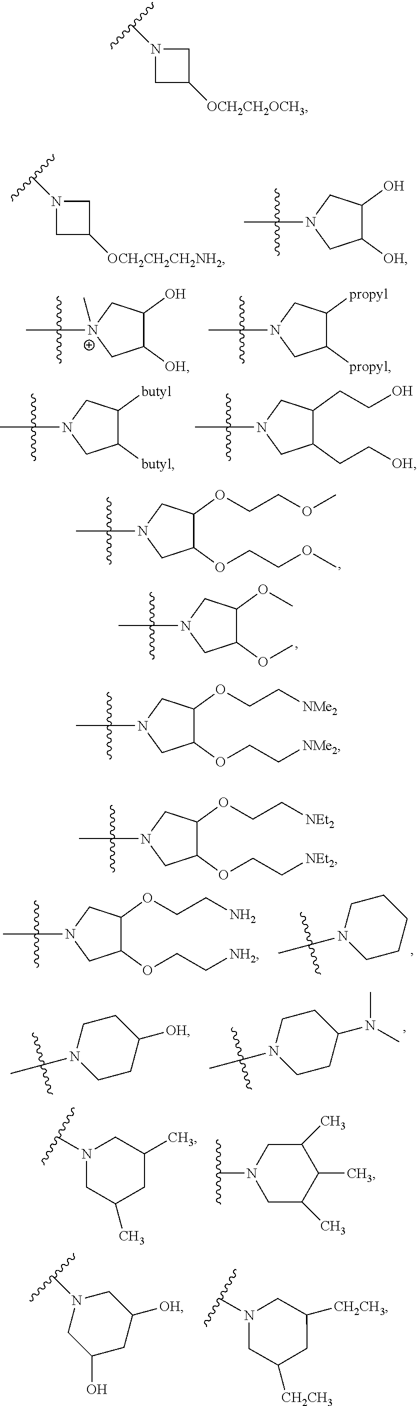

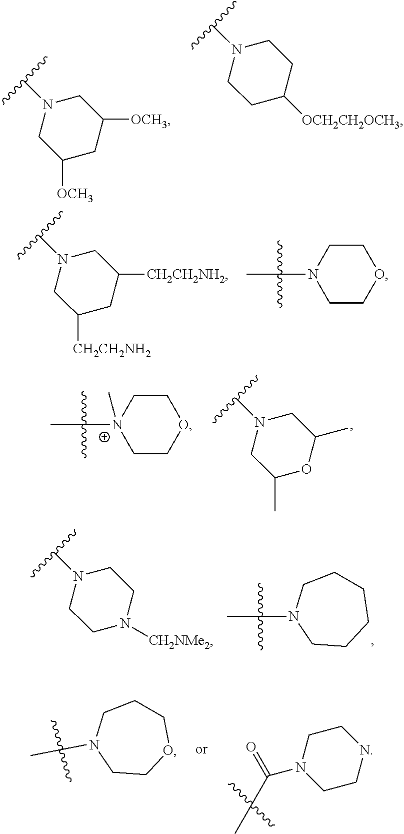

In some embodiments, the ionizable lipid is represented by formula of

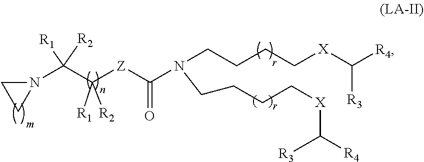

pharmaceutically acceptable salts thereof, and stereoisomers of any of the foregoing, wherein
R₁ is each independently H, C₁-C₃ alkyl, OH, halogen, SH, or NR₁₀R₁₁; R₁ and R₂ can be taken together to form a cyclic ring; R₁₀ and R₁₁ are each independently H, C₁-C₃ alkyl, and R₁₀ and R₁₁ can be taken together to form a heterocyclic ring;
R₂ is each independently H, C₁-C₃ alkyl, OH, halogen, SH, or NR₁₀R₁₁; R₁ and R₂ can be taken together to form a cyclic ring; R₁₀ and R₁₁ are each independently H, C₁-C₃ alkyl, and R₁₀ and R₁₁ can be taken together to form a heterocyclic ring;
m is 1, 2, 3, 4, 5, 6, 7 or 8;
n is 0, 1, 2, 3 or 4;
r is each independently 0, 1, 2, 3, 4, 5, 6, 7 or 8;
R₃ is each independently H, or C₃-C₁₀ alkyl;
R₄ is each independently H, or C₃-C₁₀ alkyl; provided that at least one of R₃ and R₄ is not H;
Z is absent, O, S, or NR₁₂; wherein R₁₂ is C₁-C₇ alkyl;
each X is independently X′,

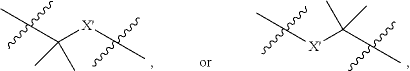

provided that at least one X in the formula is

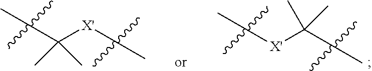

and
X′ is a biodegradable moiety.

In some embodiments, each X is

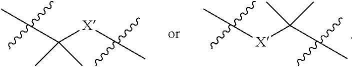

In some embodiments, X′ is —OCO—, —COO—, —NR⁷CO—, —CONR⁷—, —C(O—R₁₃)—O-(acetal), —COO(CH₂)ₛ—, —CONH(CH₂)ₛ—, —C(O—R₁₃)—O—(CH₂)ₛ—; wherein R⁷ is H or C₁-C₃ alkyl; and R₁₃ is C₃-C₁₀ alkyl.
In some embodiments, at least one X in the formula is

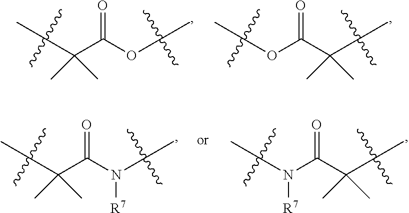

wherein R⁷ is H or methyl. In one embodiment, each X is

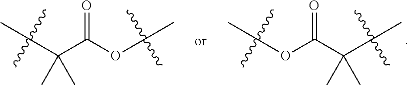

In one embodiment, each X is

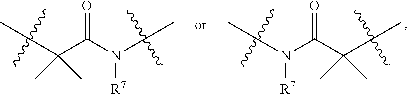

wherein R⁷ is H or methyl.

In some embodiments, m=3. In some embodiments, n=0 or 1. In some embodiments, each R R₁ and R₂ is H. In some embodiments, Z is absent.

In some embodiments, Z is S. In some embodiments, Z is O. In some embodiments, Z is NH.

In some embodiments, r is 3. In some embodiments, r is 4.

More embodiments of the above ionizable lipids comprising at least one head group (e.g., head group of formula (HA-I), (HA-III), (HA-V), (HA-VI), (HB—I), and (HC—I)), and at least one tail group of formula (TI) or (T1′) (e.g., tail group of formula (TII), (TIII), TIV, TV, TII′, or TIII′), in the Ionizable lipid compounds group iv), may be found in PCT Application No. PCT/US23/31669, filed on Aug. 31, 2023, the content of which is incorporated herein by reference in its entirety. Moreover, all the ionizable lipids of formulas (LA-I)-(LA-VII), (LB-I)-(LB-VII), (LC-IA)-(LC-IC), (LC-IIA)-(LC-IIC), and (LC-IIIA)-(LC-IIIE) of PCT Application No. PCT/US23/31669, filed on Aug. 31, 2023 are suitable for use as the ionizable lipids in this disclosure, and are incorporated herein by reference in its entirety.

Certain exemplary ionizable lipid compounds disclosed herein are set forth in Table IV below.

TABLE IV
Exemplary ionizable lipid compounds.

| Lipid | | |
|---|---|---|
| No. | Structure | Name |
| 2243 | 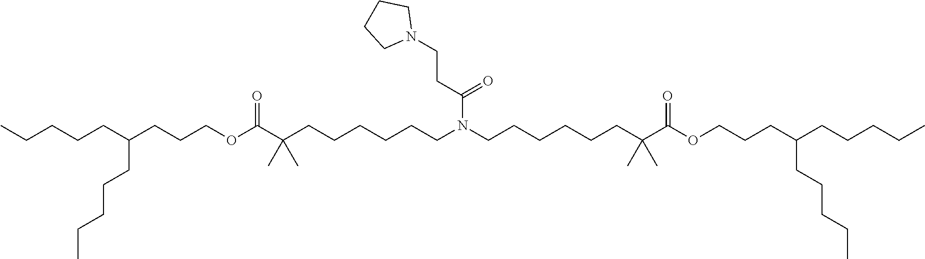 [SMILES: CCCCCC(CCCCC)CCCOC(=O)C(C)(C)CCCCCCN(CCCCCCC(C)(C)C(=O)OCCCC(CCCCC)CCCCC)C(=O)CCN1CCCC1] | bis(4-pentylnonyl) 8,8′- ((3-(pyrrolidin-1- yl)propanoyl)azanediyl) bis(2,2- dimethyloctanoate) |
| 2330 | 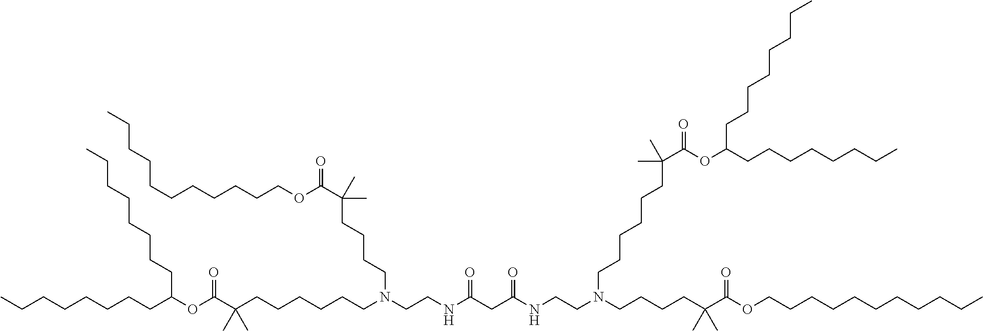 [SMILES: CCCCCCCCCCCOC(=O)C(C)(C)CCCCN(CCCCCCC(C)(C)C(=O)OC(CCCCCCCC)CCCCCCCC)CCNC(=O)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,19-bis(5,5-dimethyl- 6-oxo-6- (undecyloxy)hexyl)- 2,2,26,26-tetramethyl- 13,15-dioxo- 9,12,16,19- tetraazaheptacosanedi- oate |
| 2331 | 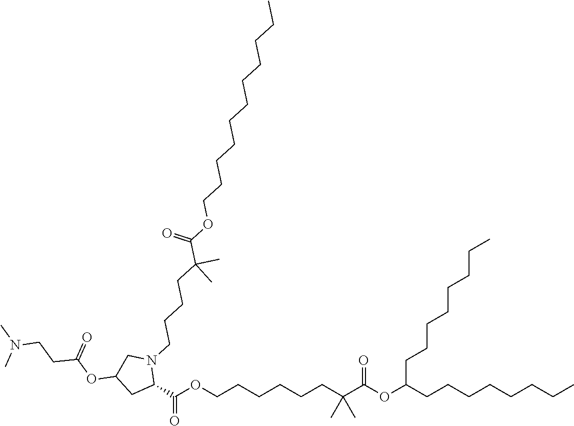 [SMILES: CCCCCCCCCCCOC(=O)C(C)(C)CCCCN1CC(OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl-8- oxooctyl (2S)-1-(5,5- dimethyl-6-oxo-6- (undecyloxy)hexyl)-4- ((3- (dimethylamino)propa- noyl)oxy)pyrrolidine-2- carboxylate |
| 2333 | 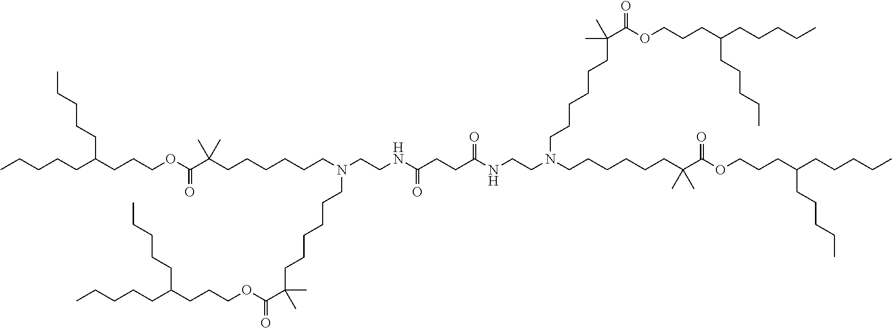 [SMILES: CCCCCC(CCCCC)CCCOC(=O)C(C)(C)CCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | bis(4-pentylnonyl) 9,20-bis(7,7-dimethyl- 8-oxo-8-((4- pentylnonyl)oxy)octyl)- 2,2,27,27-tetramethyl- 13,16-dioxo- 9,12,17,20- tetraazaoctacosanedio- ate |
| 2335 | 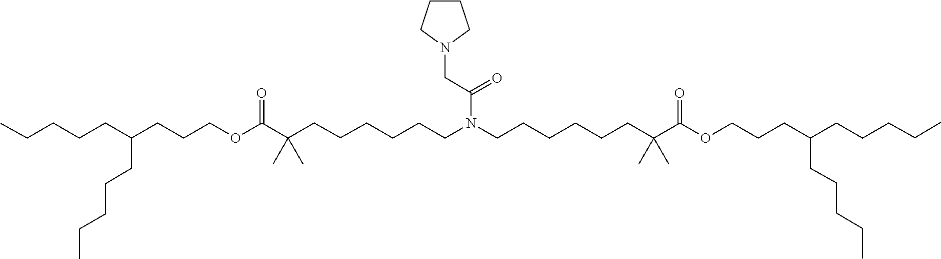 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCCC1] | bis(4-pentylnonyl) 8,8′- ((2-(pyrrolidin-1- yl)acetyl)azanediyl)bis(2,2- dimethyloctanoate) |
| 2365 | 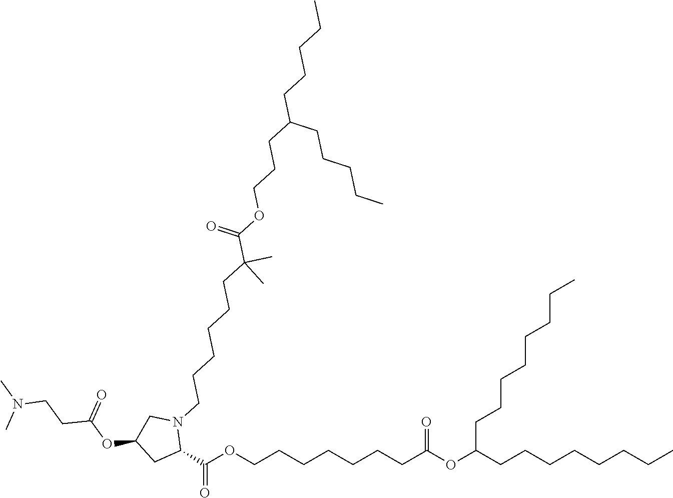 [SMILES: CCCCCCCCC(CCCCCCCC)OC(=O)CCCCCCCOC(=O)[C@@H]1C[C@@H](OC(=O)CCN(C)C)CN1CCCCCCC(C)(C)C(=O)OCCCC(CCCCC)CCCCC] | 8-(heptadecan-9- yloxy)-8-oxooctyl (2S)- 1-(7,7-dimethyl-8-oxo- 8-((4- pentylnonyl)oxy)octyl)- 4-((3- (dimethylamino)propa- noyl)oxy)pyrrolidine-2- carboxylate |
| 2366 | 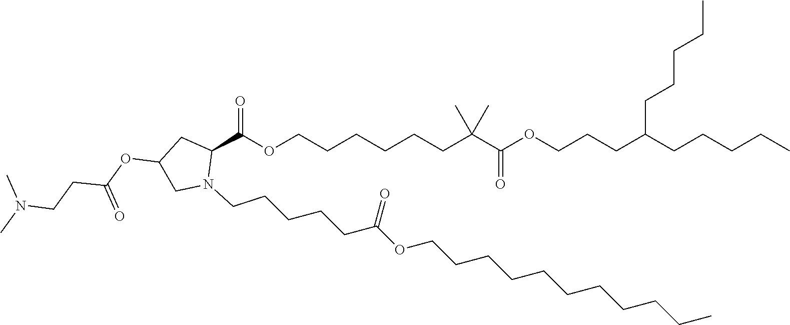 [SMILES: CCCCCCCCCCCOC(=O)CCCCCN1CC(OC(=O)CCN(C)C)C[C@H]1C(=O)OCCCCCCC(C)(C)C(=O)OCCCC(CCCCC)CCCCC] | 7,7-dimethyl-8-oxo-8- ((4- pentylnonyl)oxy)octyl (2S)-4-((3- (dimethylamino)propa- noyl)oxy)-1-(6-oxo-6- (undecyloxy)hexyl)pyrro- lidine-2-carboxylate |
| 2367 | 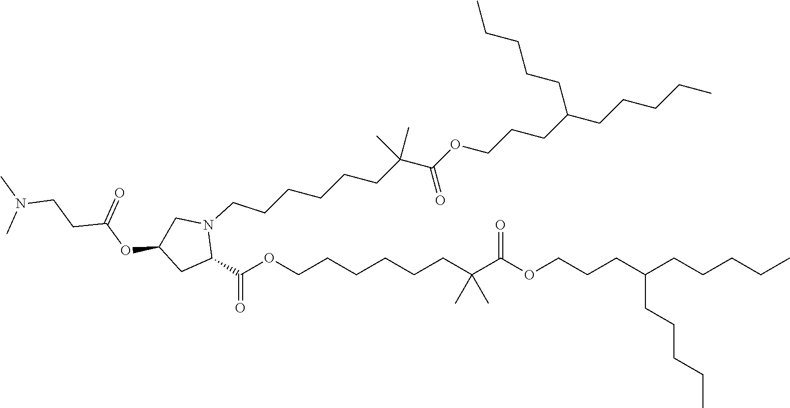 [SMILES: CCCCCC(CCCCC)CCCOC(=O)C(C)(C)CCCCCCOC(=O)[C@@H]1C[C@@H](OC(=O)CCN(C)C)CN1CCCCCCC(C)(C)C(=O)OCCCC(CCCCC)CCCCC] | 7,7-dimethyl-8-oxo-8- ((4- pentylnonyl)oxy)octyl (2S)-1-(7,7-dimethyl-8- oxo-8-((4- pentylnonyl)oxy)octyl)- 4-((3- (dimethylamino)propa- noyl)oxy)pyrrolidine-2- carboxylate |
| 2368 | 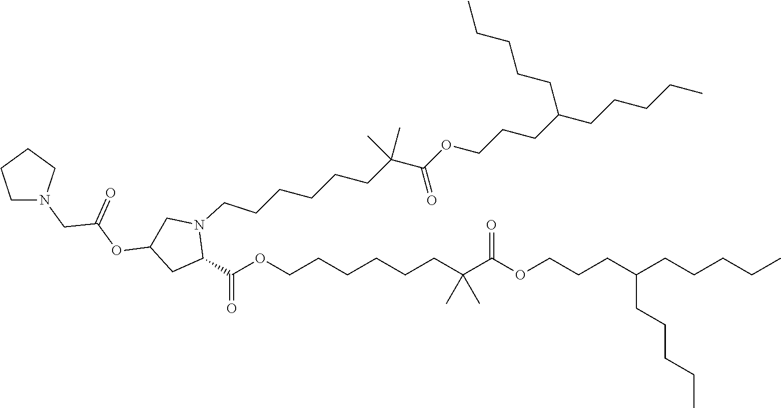 [SMILES: CCCCCC(CCCCC)CCCOC(=O)C(C)(C)CCCCCCOC(=O)[C@@H]1CC(OC(=O)CN2CCCC2)CN1CCCCCCC(C)(C)C(=O)OCCCC(CCCCC)CCCCC] | 7,7-dimethyl-8-oxo-8- ((4- pentylnonyl)oxy)octyl (2S)-1-(7,7-dimethyl-8- oxo-8-((4- pentylnonyl)oxy)octyl)- 4-(2-(pyrrolidin-1- yl)oacetoxy)pyrrolidine- 2-carboxylate |
| 2369 | 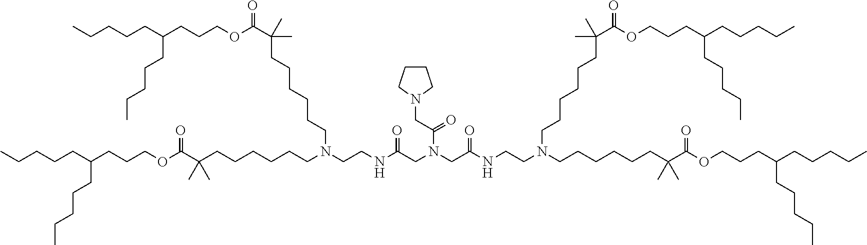 [SMILES: CCCCCC(CCCCC)CCCOC(=O)C(C)(C)CCCCCCN(CCCCCCC(C)(C)C(=O)OCCCC(CCCCC)CCCCC)CCNC(=O)CN(CC(=O)NCCN(CCCCCCC(C)(C)C(=O)OCCCC(CCCCC)CCCCC)CCCCCCC(C)(C)C(=O)OCCCC(CCCCC)CCCCC)C(=O)CN1CCCC1] | bis(4-pentylnonyl) 9,21-bis(7,7-dimethyl- 8-oxo-8-((4- pentylnonyl)oxy)octyl)- 2,2,28,28-tetramethyl- 13,17-dioxo-15-(2- (pyrrolidin-1-yl)acetyl)- 9,12,15,18,21- pentaazanonacosanedi- oate |
| 2383 | 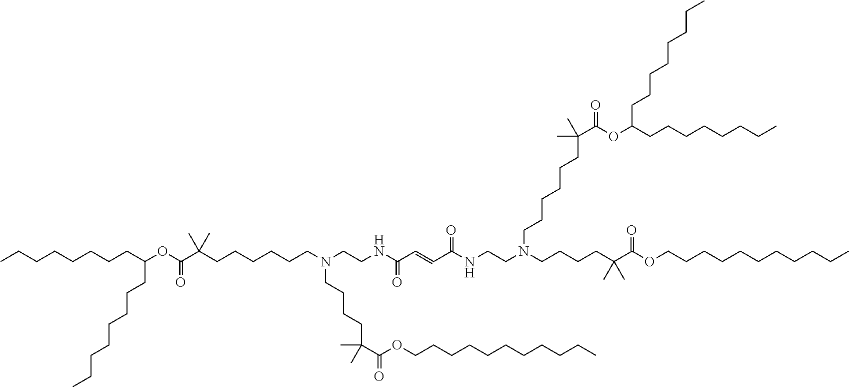 [SMILES: CCCCCCCCCCCOC(=O)C(C)(C)CCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) (E)-9,20-bis(5,5- dimethyl-6-oxo-6- (undecyloxy)hexyl)- 2,2,27,27-tetramethyl- 13,16-dioxo- 9,12,17,20- tetraazaoctacos-14- enedioate |
| 2392 | 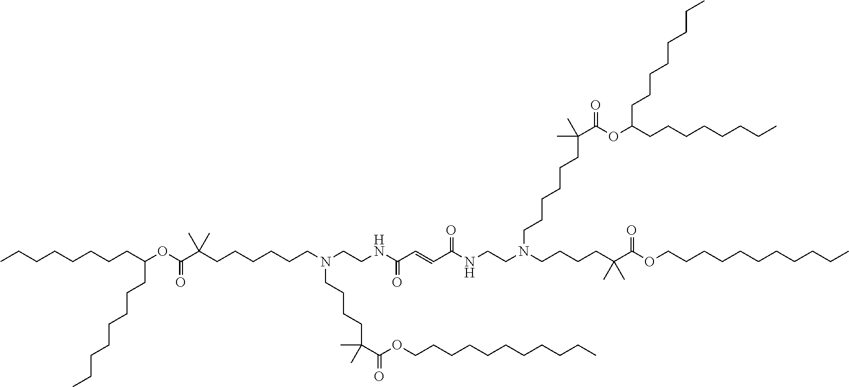 [SMILES: CCCCCCCCCCCOC(=O)C(C)(C)CCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,20-bis(5,5-dimethyl- 6-oxo-6- (undecyloxy)hexyl)- 2,2,27,27-tetramethyl- 13,16-dioxo- 9,12,17,20- tetraazaoctacosanedio- ate |
| 2398 | 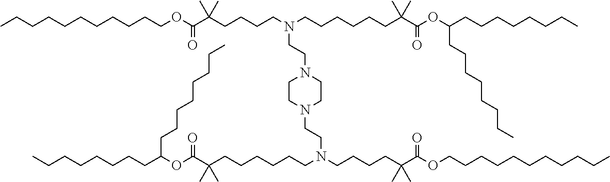 | di(heptadecan-9-yl) 8,8′-((piperazine-1,4- diylbis(ethane-2,1- diyl))bis((5,5-dimethyl- 6-oxo-6- (undecyloxy)hexyl)azane- diyl))bis(2,2- dimethyloctanoate) |
| 2424 | 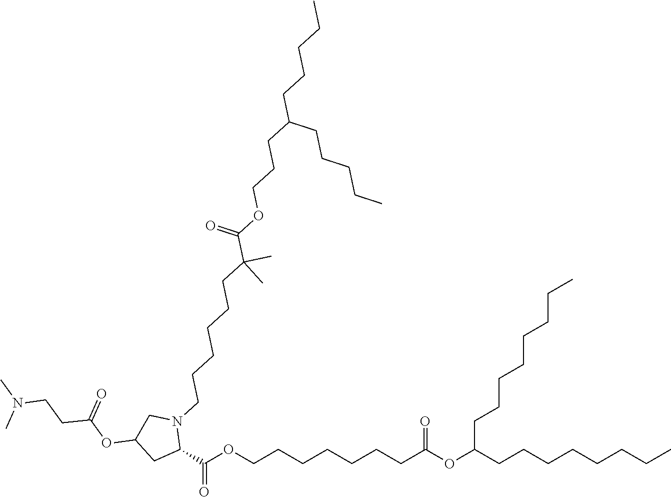 [SMILES: CCCCCCCCC(CCCCCCCC)OC(=O)CCCCCCCOC(=O)[C@@H]1CC(OC(=O)CCN(C)C)CN1CCCCCCC(C)(C)C(=O)OCCCC(CCCCC)CCCCC] | 8-(heptadecan-9- yloxy)-8-oxooctyl (2S,4R)-1-(7,7- dimethyl-8-oxo-8-((4- pentylnonyl)oxy)octyl)- 4-((3- (dimethylamino)propa- noyl)oxy)pyrrolidine-2- carboxylate |
| 2425 | 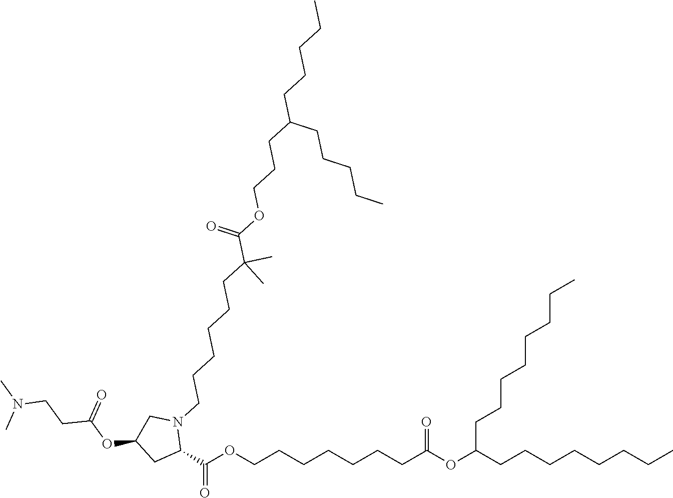 [SMILES: CCCCCCCCC(CCCCCCCC)OC(=O)CCCCCCCOC(=O)[C@@H]1C[C@@H](OC(=O)CCN(C)C)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3- (dimethylamino)propa- noyl)oxy)pyrrolidine-2- carboxylate |
| 2432 | 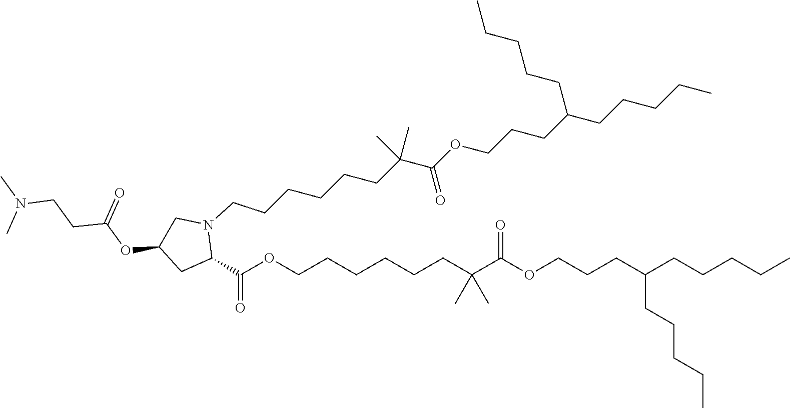 [SMILES: CCCCCC(CCCCC)CCCOC(=O)C(C)(C)CCCCCCOC(=O)[C@@H]1C[C@@H](OC(=O)CCN(C)C)CN1CCCCCCC(C)(C)C(=O)OCCCC(CCCCC)CCCCC] | 7,7-dimethyl-8-oxo-8- ((4- pentylnonyl)oxy)octyl (2S,4R)-1-(7,7- dimethyl-8-oxo-8-((4- pentylnonyl)oxy)octyl)- 4-((3- (dimethylamino)propa- noyl)oxy)pyrrolidine-2- carboxylate |
| 2433 | 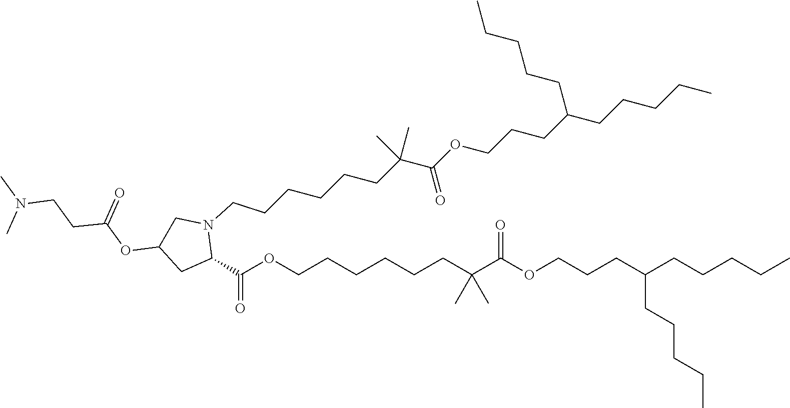 [SMILES: CCCCCC(CCCCC)CCCOC(=O)C(C)(C)CCCCCCOC(=O)[C@@H]1CC(OC(=O)CCN(C)C)CN1CCCCCCC(C)(C)C(=O)OCCCC(CCCCC)CCCCC] | 7,7-dimethyl-8-oxo-8- ((4- pentylnonyl)oxy)octyl (2S,4S)-1-(,7,7- dimethyl-8-oxo-8-((4- pentylnonyl)oxy)octyl)- 4-((3- (dimethylamino)propa- noyl)oxy)pyrrolidine-2- carboxylate |
| 2441 | 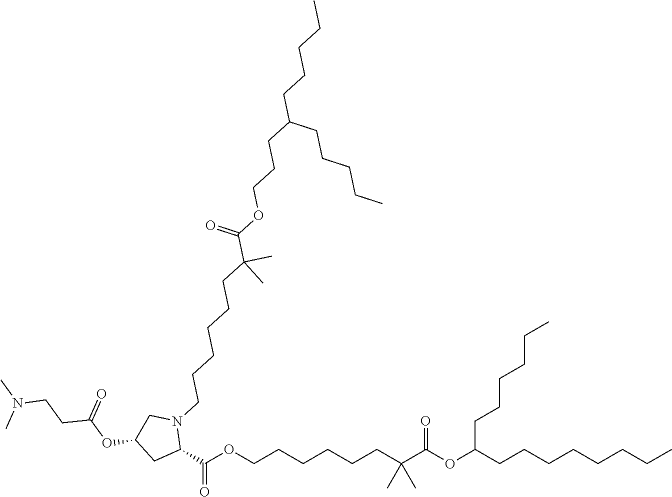 [SMILES: CCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | 7,7-dimethyl-8-oxo-8- (pentadecan-7- yloxy)octyl (2S,4S)-1- (7,7-dimethyl-8-oxo-8- ((4- pentylnonyl)oxy)octyl)- 4-((3- (dimethylamino)propa- noyl)oxy)pyrrolidine-2- carboxylate |
| 2442 | 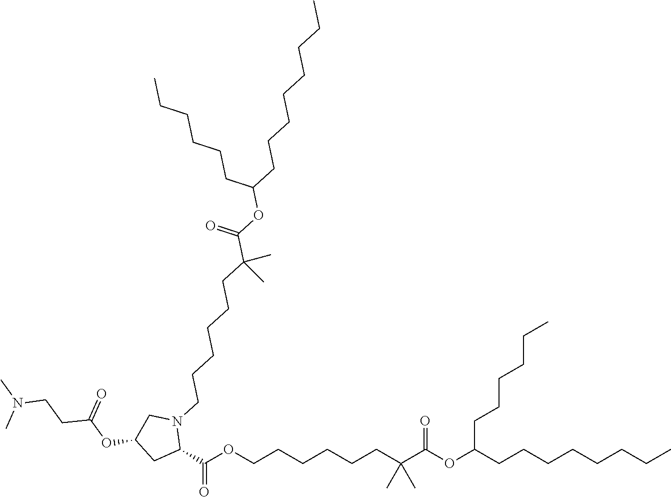 [SMILES: CCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC] | 7,7-dimethyl-8-oxo-8- (pentadecan-7- yloxy)octyl (2S,4S)-1- (7,7-dimethyl-8-oxo-8- (pentadecan-7- yloxy)octyl)-4-((3- (dimethylamino)propa- noyl)oxy)pyrrolidine-2- carboxylate |
| 2443 | 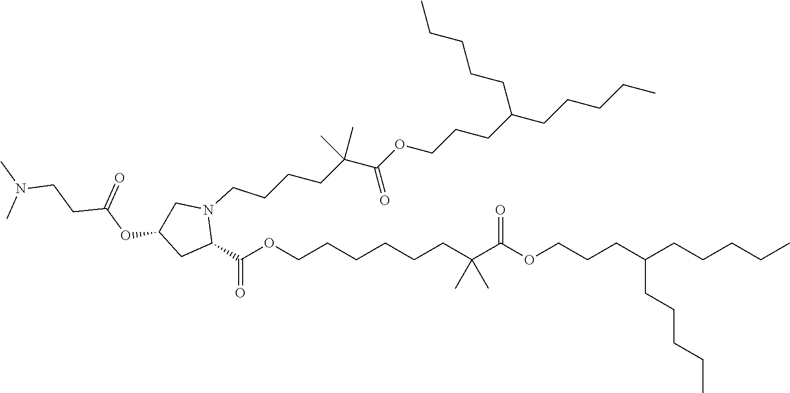 [SMILES: CCCCCC(CCCCC)CCCOC(=O)C(C)(C)CCCCCCOC(=O)[C@@H]1C[C@H](OC(=O)CCN(C)C)CN1CCCCC(C)(C)C(=O)OCCCC(CCCCC)CCCCC] | 7,7-dimethyl-8-oxo-8- ((4- pentylnonyl)oxy)octyl (2S,4S)-1-(5,5- dimethyl-6-oxo-6-((4- pentylnonyl)oxy)hexyl)- 4-((3- (dimethylamino)propa- noyl)oxy)pyrrolidine-2- carboxylate |
| 2454 | 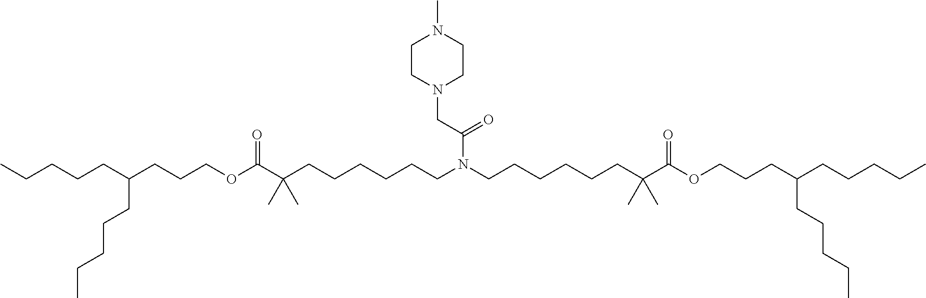 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCN(C)CC1] | bis(4-pentylnonyl) 8,8′- ((2-(4-methylpiperazin- 1- yl)acetyl)azanediyl)bis(2,2- dimethyloctanaote) |
| 2481 | 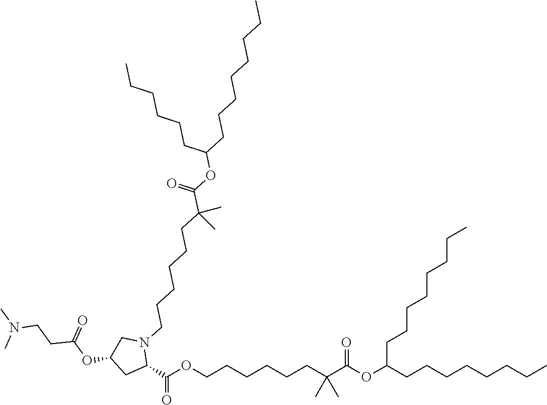 [SMILES: CCCCCCCCC(CCCCCC)OC(=O)C(C)(C)CCCCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl-8- oxooctyl (2S,4S)-1- (7,7-dimethyl-8-oxo-8- (pentadecan-7- yloxy)octyl)-4-((3- (dimethylamino)propa- noyl)oxy)pyrrolidine-2- carboxylate |
| 2483 | 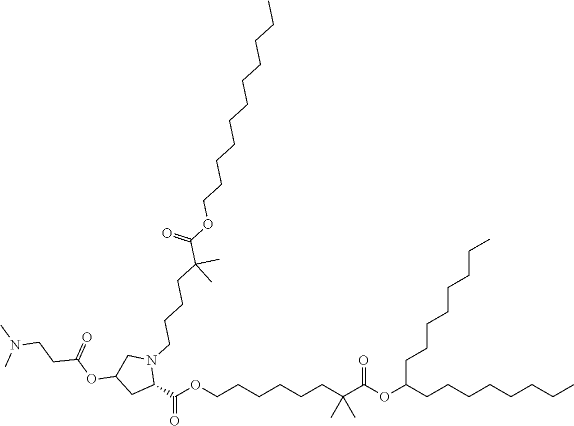 [SMILES: CCCCCCCCCCCOC(=O)C(C)(C)CCCCN1CC(OC(=O)CCN(C)C)C[C@H]1C(=O)OCCCCCCC(C)(C)C(=O)OC(CCCCCCCC)CCCCCCCC] | 7,7-dimethyl-8-oxo-8- (pentadecan-7- yloxy)octyl (2S,4S)-1- (5,5-dimethyl-6-oxo-6- (undecyloxy)hexyl)-4- ((3- (dimethylamino)propa- noyl)oxy)pyrrolidine-2- carboxylate |
| 2370 | 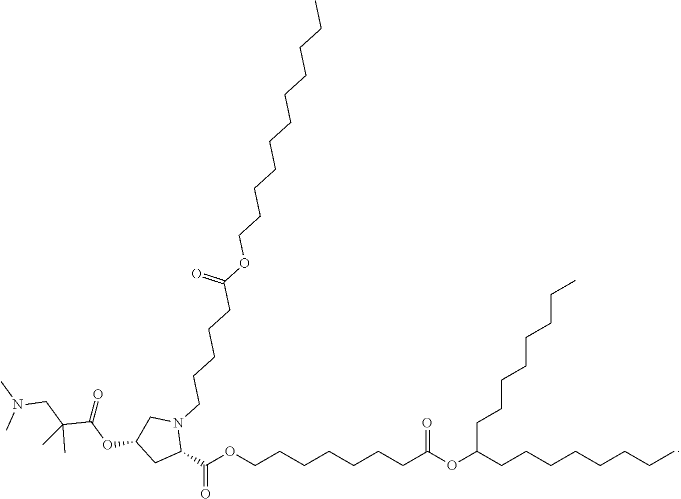 [SMILES: CCCCCCCCCCCOC(=O)CCCCCN1C[C@@H](OC(=O)C(C)(C)CN(C)C)C[C@H]1C(=O)OCCCCCCCC(=O)OC(CCCCCCCC)CCCCCCCC] | 8-(heptadecan-9- yloxy)-8-oxooctyl (2S)- 4-((3-(dimethylamino)- 2,2- dimethylpropanoyl)oxy)- 1-(6-oxo-6- (undecyloxy)hexyl)pyrro- lidine-2-carboxylate |
| 2511 | 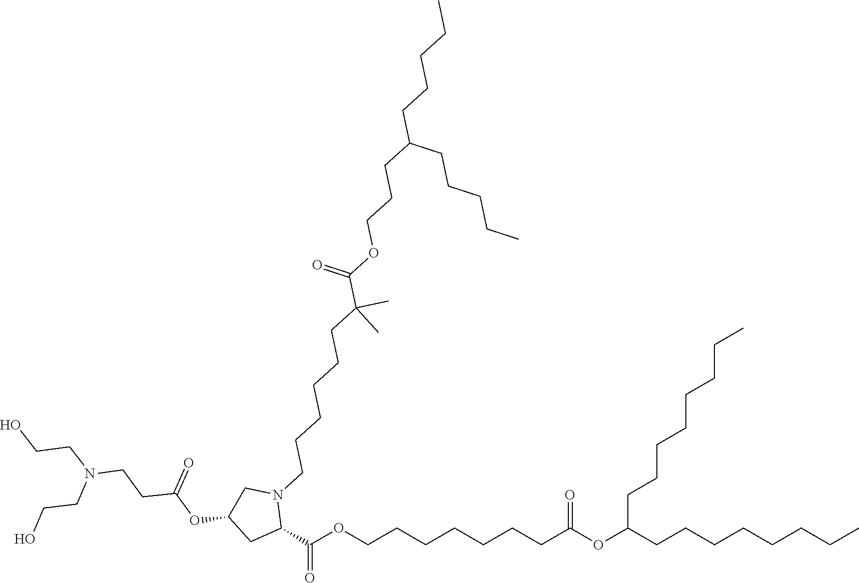 [SMILES: CCCCCCCCC(CCCCCCCC)OC(=O)CCCCCCCOC(=O)[C@@H]1C[C@H](OC(=O)CCN(CCO)CCO)CN1CCCCCCC(C)(C)C(=O)OCCCC(CCCCC)CCCCC] | 8-(heptadecan-9- yloxy)-8-oxooctyl (2S,4S)-4-((3-(bis(2- hydroxyethyl)amino)pro- panoyl)oxy)-1-(7,7- dimethyl-8-oxo-8-((4- pentylnonyl)oxy)octyl) pyrrolidine-2- carboxylate |
| 2510 | 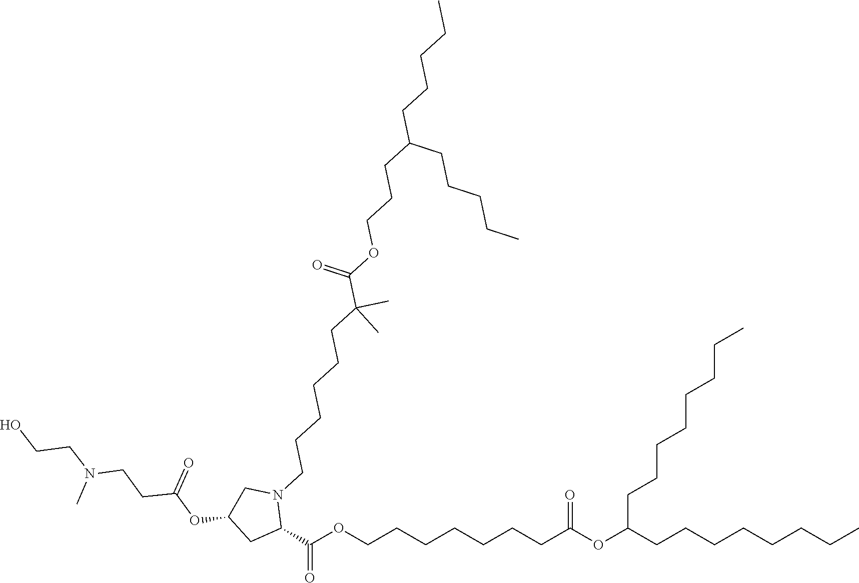 [SMILES: CCCCCCCCC(CCCCCCCC)OC(=O)CCCCCCCOC(=O)[C@@H]1C[C@H](OC(=O)CCN(C)CCO)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3-((2- hydroxyethyl)(methyl)a- mino)propanoyl)oxy)pyrro- lidine-2- carboxylate |
| 2509 | 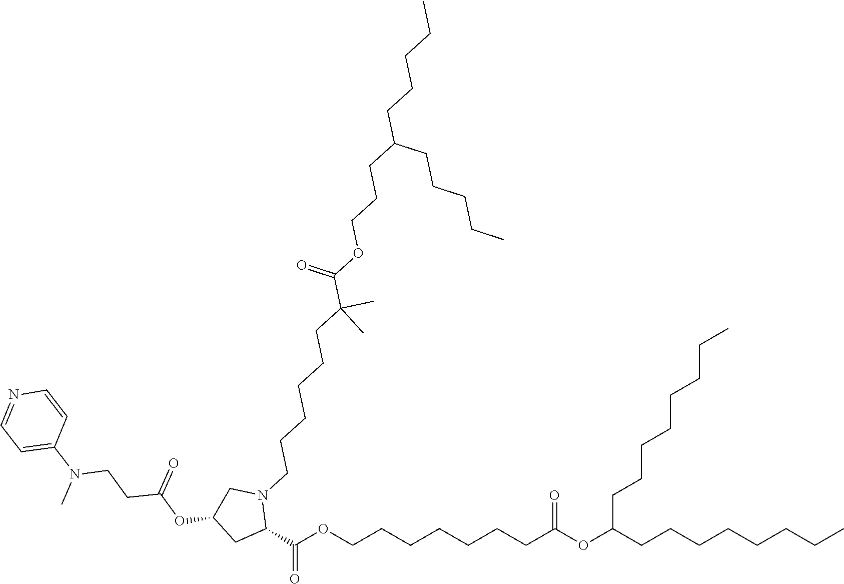 [SMILES: CCCCCCCCC(CCCCCCCC)OC(=O)CCCCCCCOC(=O)[C@@H]1C[C@H](OC(=O)CCN(C)c2ccncc2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3-(methyl(pyridin- 4- yl)amino)propanoyl)oxy) pyrrolidine-2- carboxylate |
| 2508 | 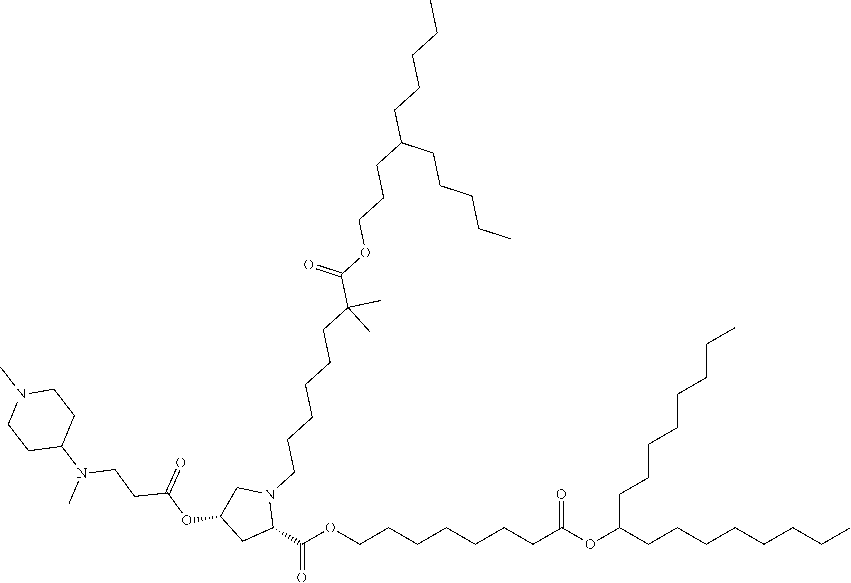 [SMILES: CCCCCCCCC(CCCCCCCC)OC(=O)CCCCCCCOC(=O)[C@@H]1C[C@H](OC(=O)CCN(C)C2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3-(methyl(1- methylpiperidin-4- yl)amino)propanoyl)oxy) pyrrolidine-2- carboxylate |
| 2507 | 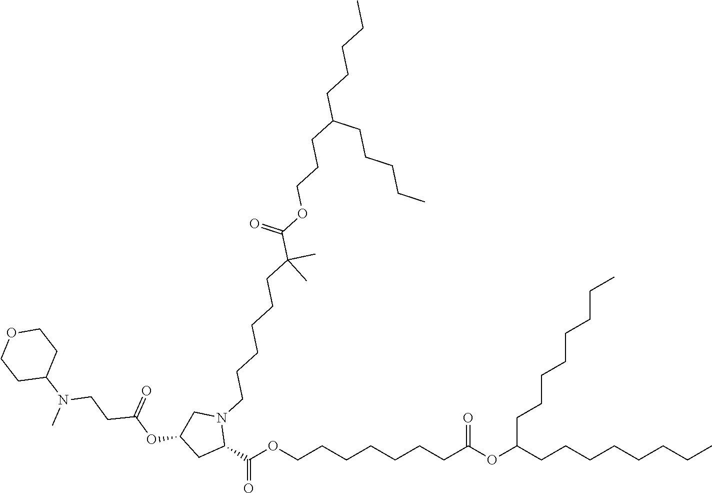 [SMILES: CCCCCCCCC(CCCCCCCC)OC(=O)CCCCCCCOC(=O)[C@@H]1C[C@H](OC(=O)CCN(C)C2CCOCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3- (methyl(tetrahydro-2H- pyran-4- y)amino)propanoyl)oxy) pyrrolidine-2- carboxylate |
| 2506 | 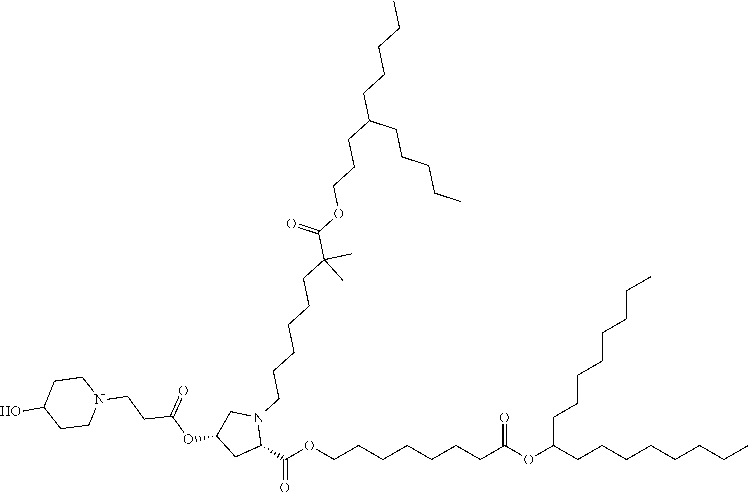 [SMILES: CCCCCCCCC(CCCCCCCC)OC(=O)CCCCCCCOC(=O)[C@@H]1C[C@H](OC(=O)CCN2CCC(O)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3-(4- hydroxypiperidin-1- yl)propanyl)oxy)pyrroli- dine-2-carboxylate |
| 2505 | 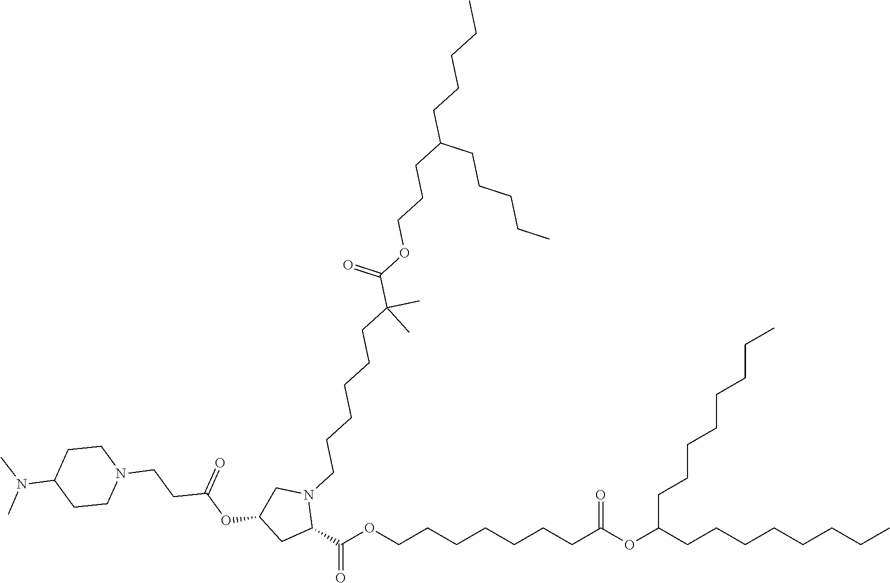 [SMILES: CCCCCCCCC(CCCCCCCC)OC(=O)CCCCCCCOC(=O)[C@@H]1C[C@H](OC(=O)CCN2CCC(N(C)C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3-(4- (dimethylamino)piperidin- 1- yl)propanoyl)oxy)pyrroli dine-2-carboxylate |
| 2504 | 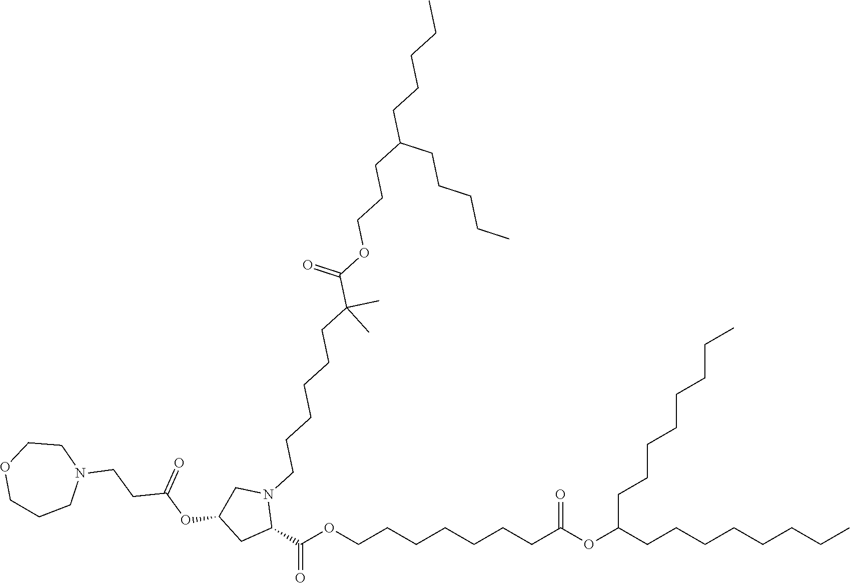 [SMILES: CCCCCCCCC(CCCCCCCC)OC(=O)CCCCCCCOC(=O)[C@@H]1C[C@H](OC(=O)CCN2CCCOCC2)CN1CCCCCCC(C)(C)C(=O)OCCCC(CCCCC)CCCCC] | 8-(heptadecan-9- ylxoy)-8-oxooctyl (2S,4S)-4-((3-(1,4- oxazepan-4- yl)propanoyl)oxy)-1- (7,7-dimethyl-8-oxo-8- ((4- pentylnonyl)oxy)octyl) pyrrolidine-2- carboxylate |
| 2503 | 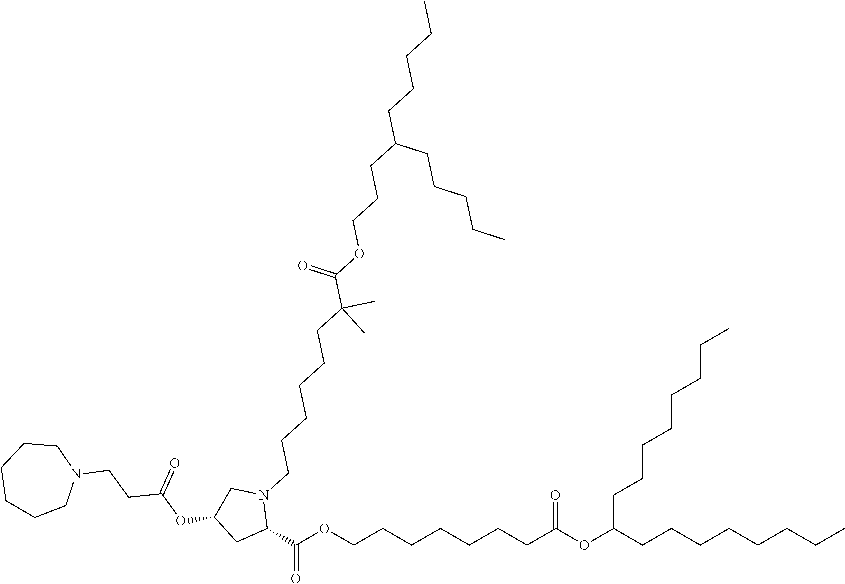 [SMILES: CCCCCCCCC(CCCCCCCC)OC(=O)CCCCCCCOC(=O)[C@@H]1C[C@H](OC(=O)CCN2CCCCCC2)CN1CCCCCCC(C)(C)C(=O)OCCCC(CCCCC)CCCCC] | 8-(heptadecan-9- yloxy)-8-oxooctyl (2S,4S)-4-((3-(azepan- 1-yl)propanoyl)oxy)-1- (7,7-dimethyl-8-oxo-8- ((4- pentylnonyl)oxy)octyl) pyrrolidine-2- carboxylate |
| 2502 | 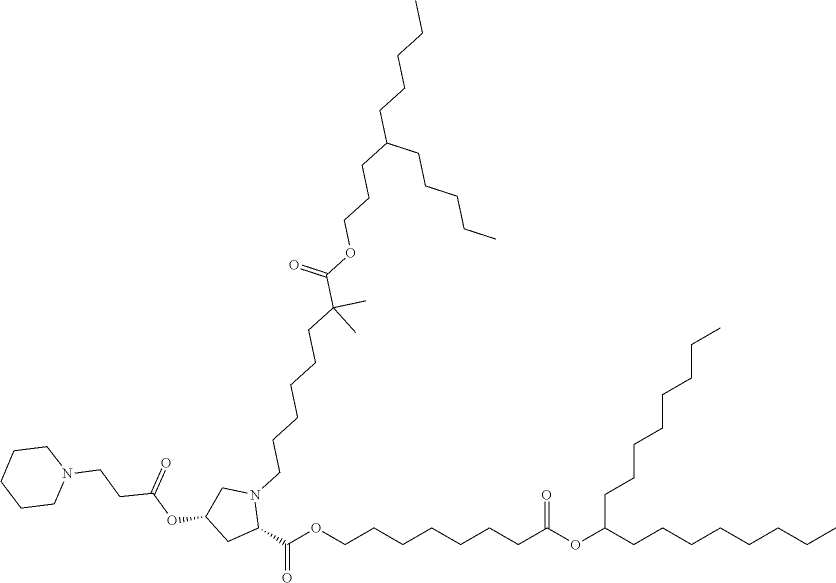 [SMILES: CCCCCCCCC(CCCCCCCC)OC(=O)CCCCCCCOC(=O)[C@@H]1C[C@H](OC(=O)CCN2CCCCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3-(piperidin-1- yl)propanoyl)oxy)pyrroli- dine-2-carboxylate |
| 2501 | 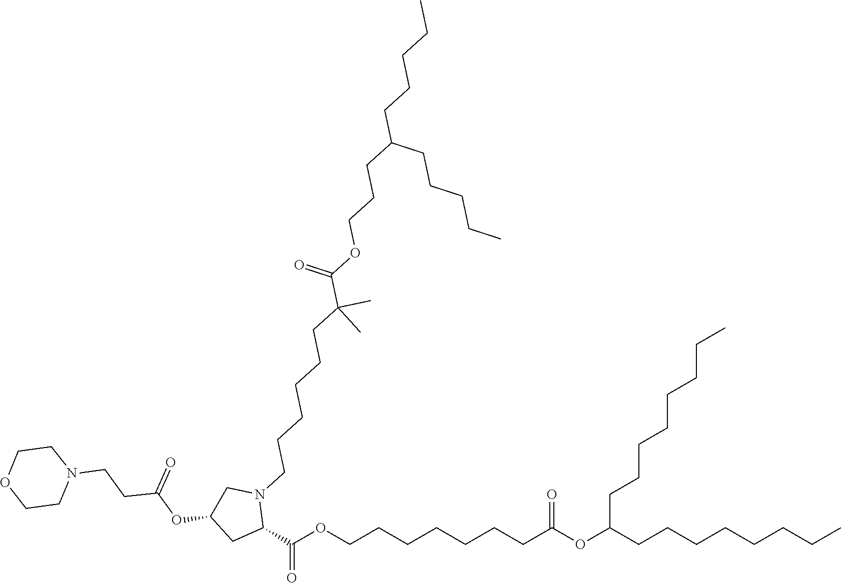 [SMILES: CCCCCCCCC(CCCCCCCC)OC(=O)CCCCCCCOC(=O)[C@@H]1C[C@H](OC(=O)CCN2CCOCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3- morpholinopropanoyl) oxy)pyrrolidine-2- carboxyalate |
| 2500 | 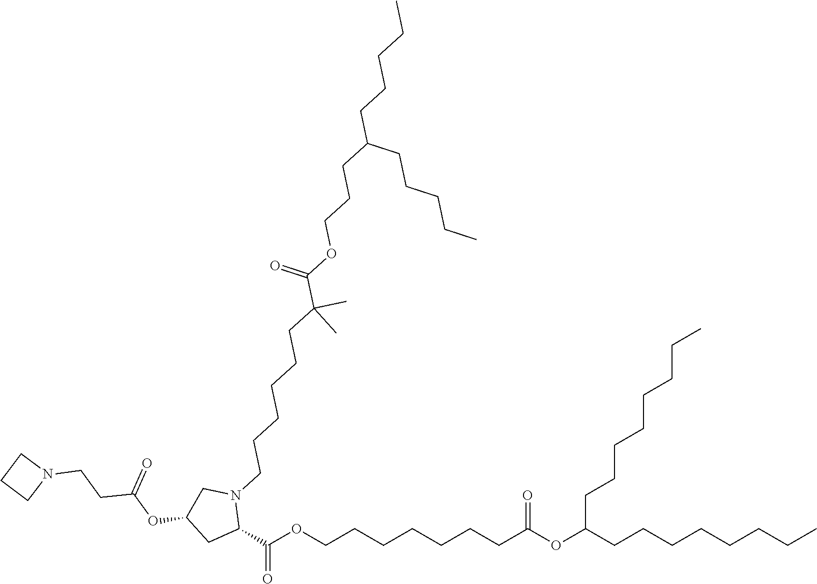 [SMILES: CCCCCCCCC(CCCCCCCC)OC(=O)CCCCCCCOC(=O)[C@@H]1C[C@H](OC(=O)CCN2CCC2)CN1CCCCCCC(C)(C)C(=O)OCCCC(CCCCC)CCCCC] | 8-(heptadecan-9- yloxy)-8-oxooctyl (2S,4S)-4-((3- (azetidin-1- yl)propanoyl)oxy)-1- (7,7-dimethyl-8-oxo-8- ((4- pentylnonyl)oxy)octyl) pyrrolidine-2- carboxylate |
| 2499 | 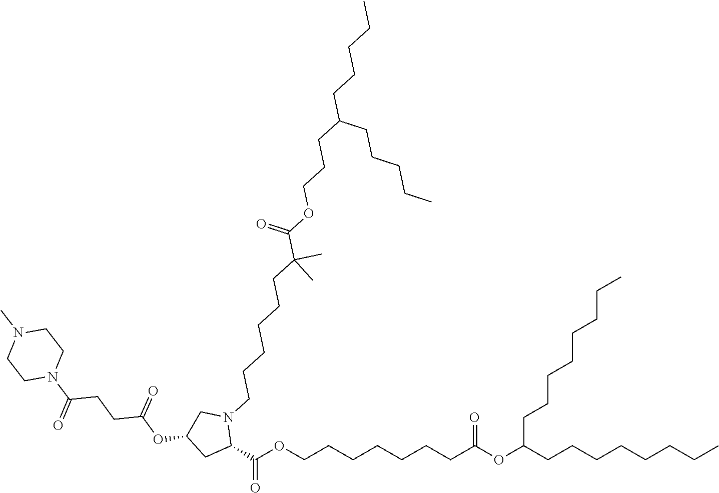 [SMILES: CCCCCCCCC(CCCCCCCC)OC(=O)CCCCCCCOC(=O)[C@@H]1C[C@H](OC(=O)CCC(=O)N2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((4-(4- methylpiperaizn-1-yl)- 4- oxobutanoyl)oxy)pyrroli- dine-2-carboxylate |
| 2498 | 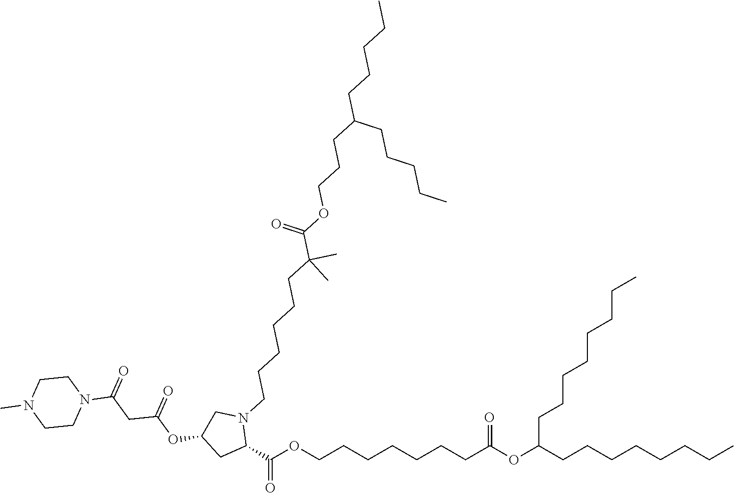 [SMILES: CCCCCCCCC(CCCCCCCC)OC(=O)CCCCCCCOC(=O)[C@@H]1C[C@H](OC(=O)CC(=O)N2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8-((4- pentylnonyl)oxy)octyl)- 4-((3-(4- methylpiperazin-1-yl)- 3- oxopropanoyl)oxy)pyrro- lidine-2-carboxylate |
| 2488 | 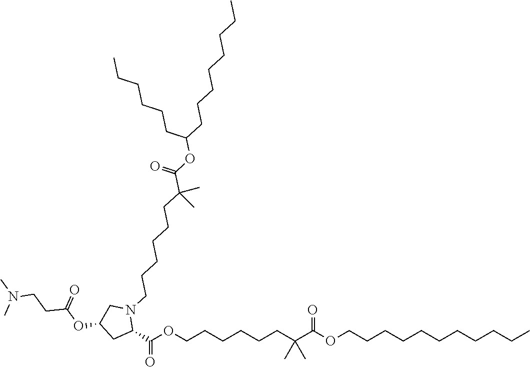 [SMILES: CCCCCCCCCCCOC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC] | 7,7-dimethyl-8-oxo-8- (undecyloxy)octyl (2S,4S)-1-(7,7- dimethyl-8-oxo-8- (pentadecan-7- yloxy)octyl)-4-((3- (dimethylamino)propa- noyl)oxy)pyrrolidine-2- carboxylate |
| 2487 | 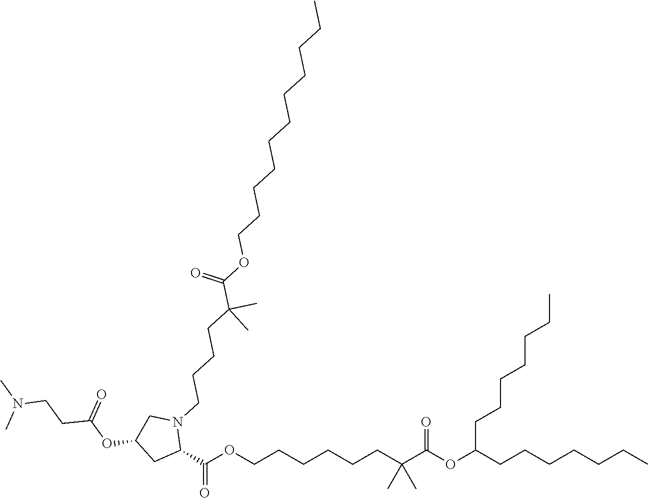 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCCC)CCCCCCC] | 7,7-dimethyl-8-oxo-8- (pentadecan-8- yloxy)octyl (2S,4S)-1- (5,5-dimethyl-6-oxo-6- (undecyloxy)hexyl)-4- ((3- (dimethylamino)propa- noyl)oxy)pyrrolidine-2- carboxylate |
| 2486 | 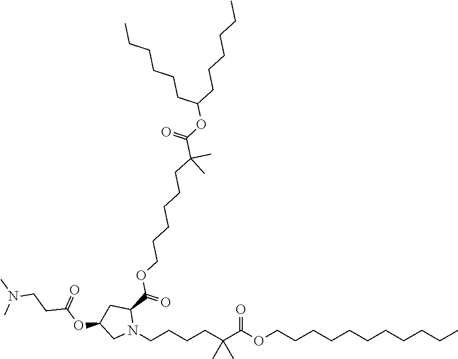 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCC)CCCCCC] | 7,7-dimethyl-8-oxo-8- (tridecan-7-yloxy)octyl (2S,4S)-1-(5,5- dimethyl-6-oxo-6- (undecyloxy)hexyl)-4- ((3- (dimethylamino)propa- noyl)oxy)pyrrolidine-2- carboxylate |
| 2485 | 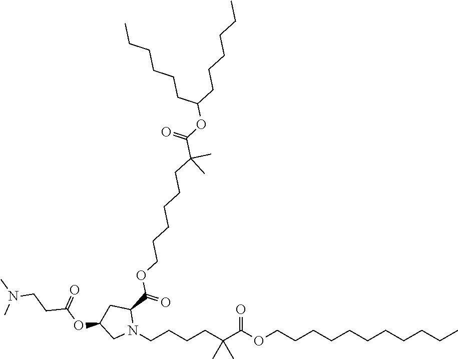 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCC)CCCCCC] | 8-(heptadecan-9- yloxy)-7,7-dimethyl-8- oxooctyl (2S,4S)-1- (7,7-dimethyl-8-oxo-8- (pentadecan-8- yloxy)octyl)-4-((3- (dimethylamino)propa- noyl)oxy)pyrrolidine-2- carboxylate |
| 2484 | 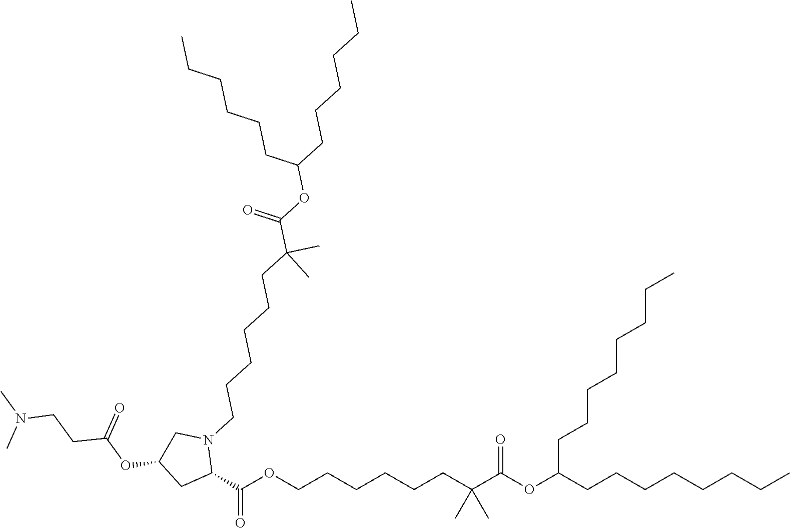 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OC(CCCCCC)CCCCCC] | 8-(heptadecan-9- yloxy)-7,7-dimethyl-8- oxooctyl (2S,4S)-1- (7,7-dimethyl-8-oxo-8- (tridecan-7- yloxy)octyl)-4-((3- (dimethylamino)propa- noyl)oxy)pyrrolidine-2- carboxylate |
| 2482 | 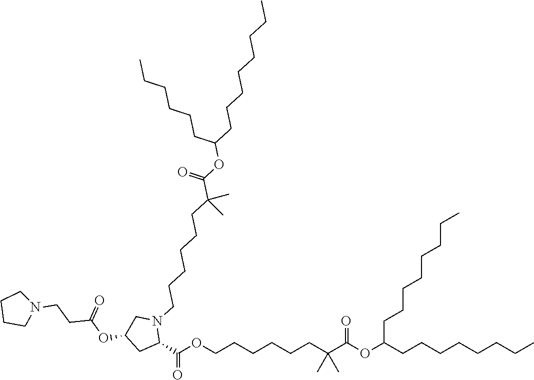 [SMILES: CCCCCCCCC(CCCCCC)OC(=O)C(C)(C)CCCCCCN1C[C@@H](OC(=O)CCN2CCCC2)C[C@H]1C(=O)OCCCCCCC(C)(C)C(=O)OC(CCCCCCCC)CCCCCCCC] | 8-(heptadecan-9- yloxy)-7,7-dimethyl-8- oxooctyl (2S,4S)-1- (7,7-dimethyl-8-oxo-8- (pentadecan-7- yloxy)octyl)-4-((3- (pyrrolidin-1- yl)propanoyl)oxy)pyrroli- dine-2-carboxylate |
| 2535 | 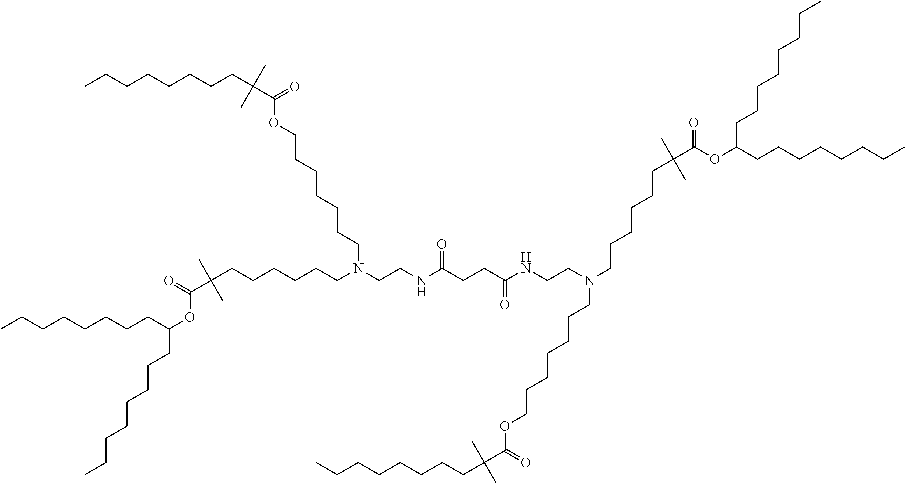 [SMILES: CCCCCCCCC(CCCCCCCC)OC(=O)C(C)(C)CCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | |
| 2536 | 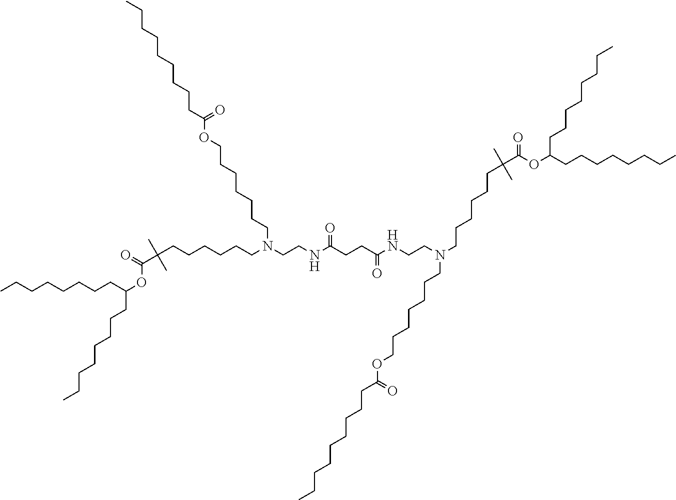 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)CCCCCCCCC)CCCCCCC(C)(C)C(=O)OC(CCCCCCCC)CCCCCCCC] | |
| 2537 | 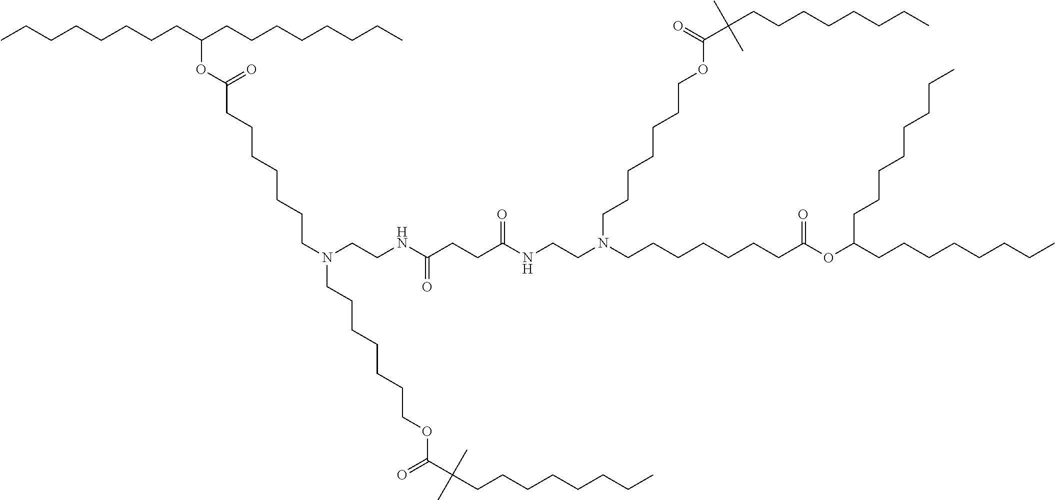 [SMILES: CCCCCCCCC(CCCCCCCC)OC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] | |
| 2544 | 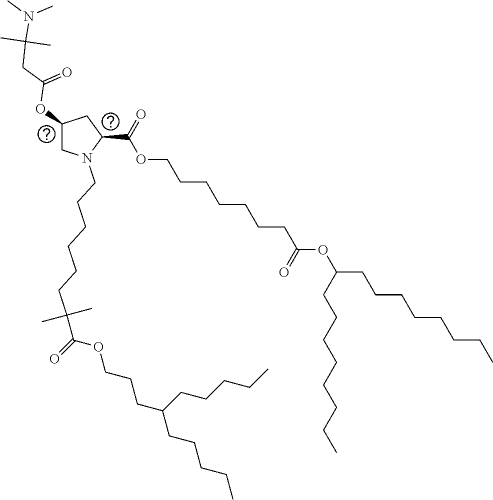 [SMILES: CCCCCCCCC(CCCCCCCC)OC(=O)CCCCCCCOC(=O)[C@@H]1C[C@H](OC(=O)CC(C)(C)N(C)C)CN1CCCCCCC(C)(C)C(=O)OCCCC(CCCCC)CCCCC] | |
| 2545 | 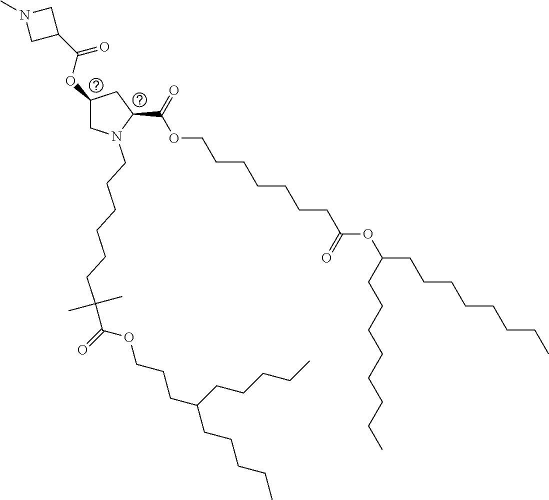 [SMILES: CCCCCCCCC(CCCCCCCC)OC(=O)CCCCCCCOC(=O)[C@@H]1C[C@H](OC(=O)C2CN(C)C2)CN1CCCCCCC(C)(C)C(=O)OCCCC(CCCCC)CCCCC] | |
| 2546 | 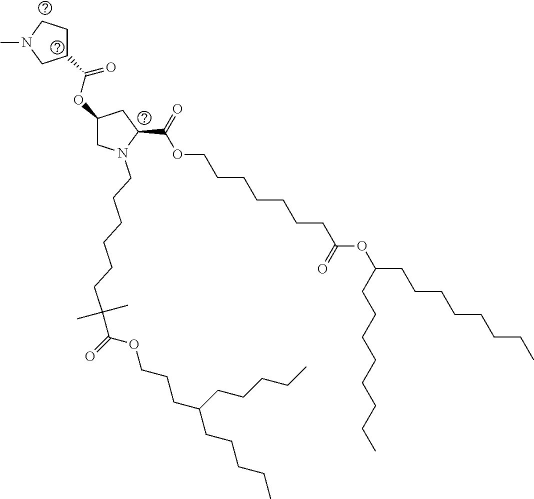 | |
| 2547 | 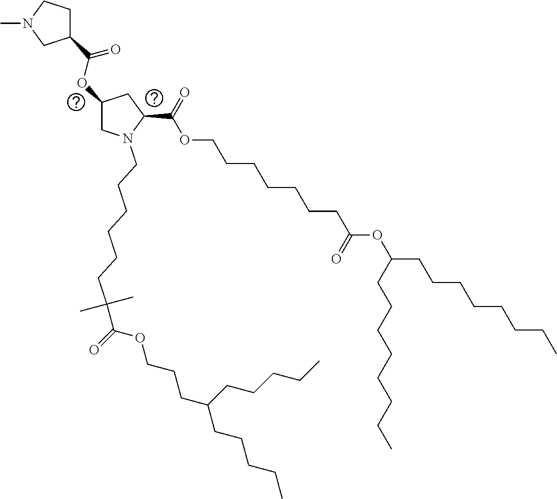 [SMILES: CCCCCCCCC(CCCCCCCC)OC(=O)CCCCCCCOC(=O)[C@@H]1C[C@H](OC(=O)[C@@H]2CCN(C)C2)CN1CCCCCCC(C)(C)C(=O)OCCCC(CCCCC)CCCCC] | |
| 2551 | 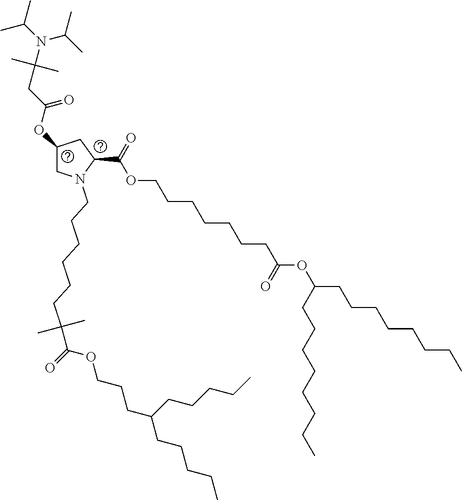 [SMILES: CCCCCCCCC(CCCCCCCC)OC(=O)CCCCCCCOC(=O)[C@@H]1C[C@H](OC(=O)CC(C)(C)N(C(C)C)C(C)C)CN1CCCCCCC(C)(C)C(=O)OCCCC(CCCCC)CCCCC] | |
| 2552 | 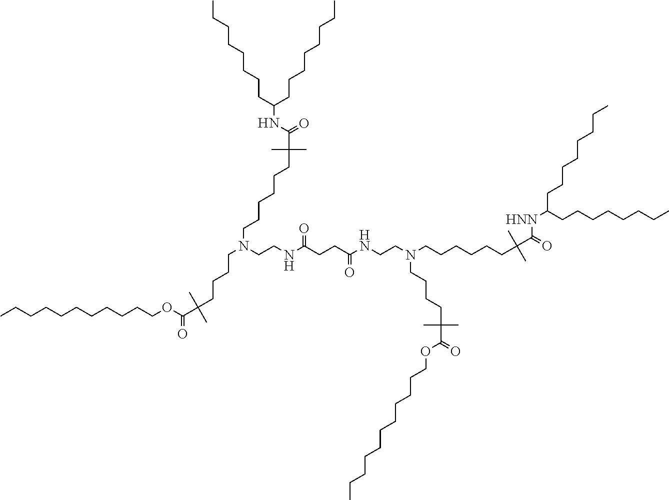 | |
| 2553 | 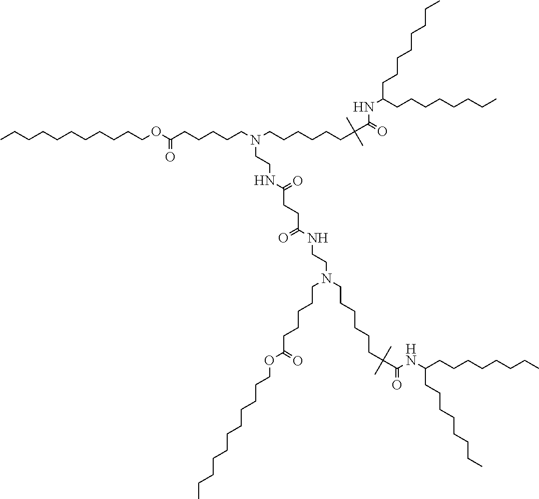 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] | |
| 2554 | 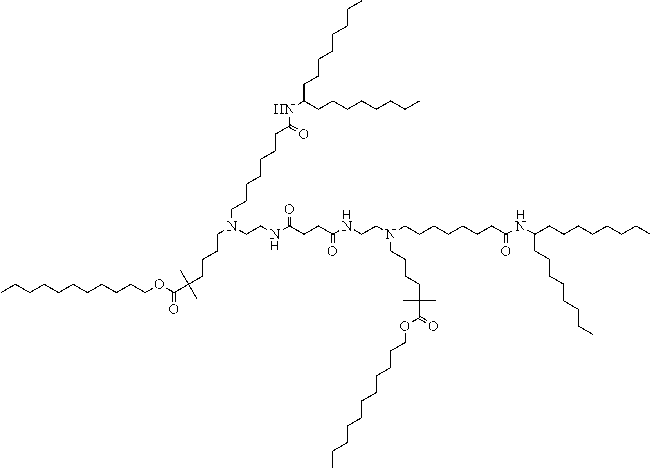 [SMILES: CCCCCCCCCCCOC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | |
| 2555 | 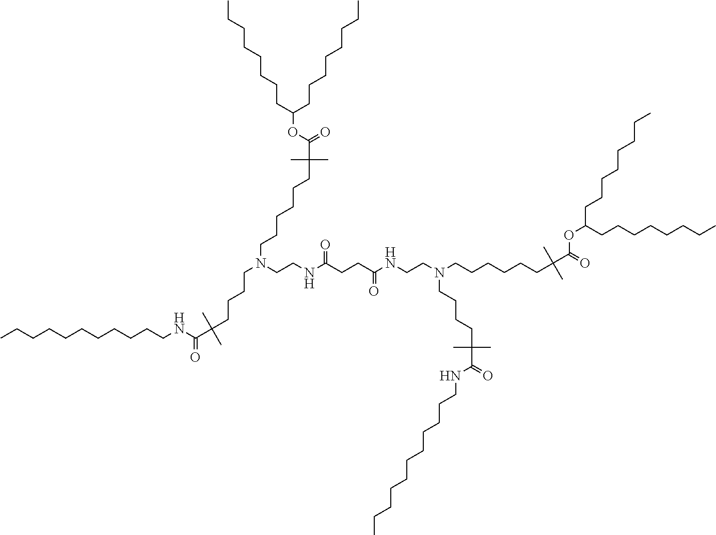 [SMILES: CCCCCCCCCCCNC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)NCCCCCCCCCCC] | |
| 2556 | 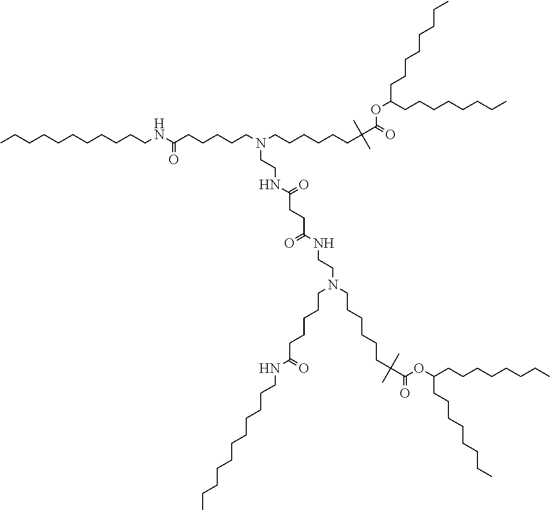 [SMILES: CCCCCCCCCCCNC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)NCCCCCCCCCCC] | |
| 2557 | 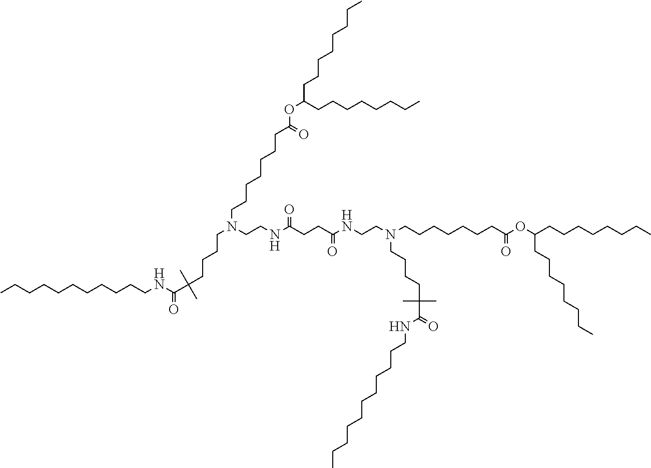 [SMILES: CCCCCCCCCCCNC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)NCCCCCCCCCCC] | |
| 2559 | 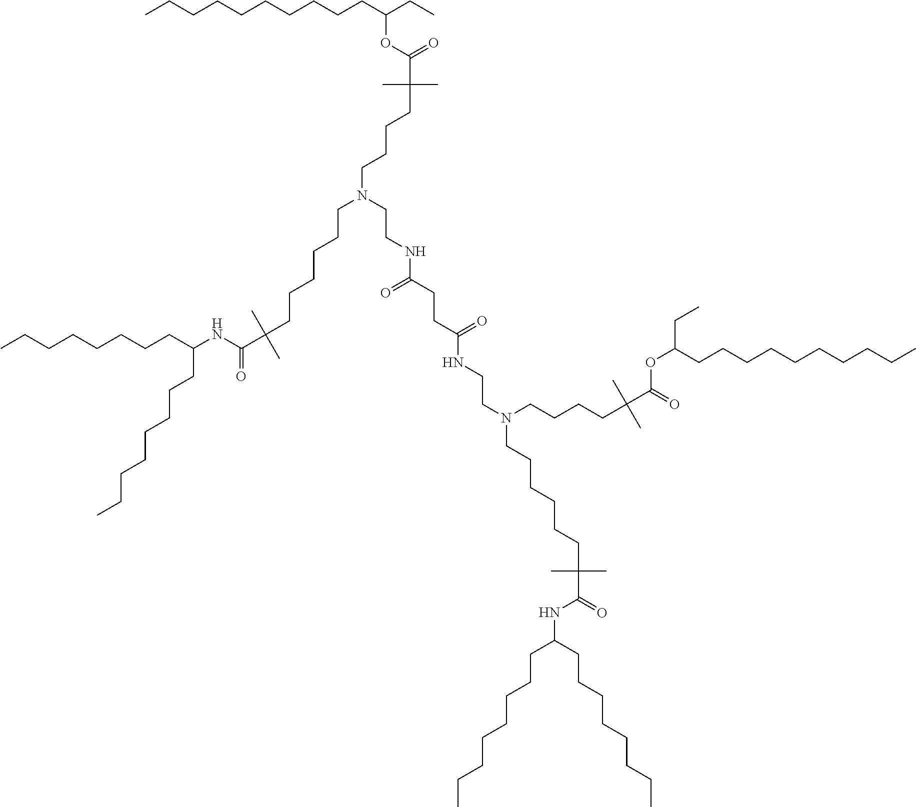 [SMILES: CCCCCCCCCCC(CC)OC(=O)C(C)(C)CCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OC(CC)CCCCCCCCCC] | |
| 2560 | 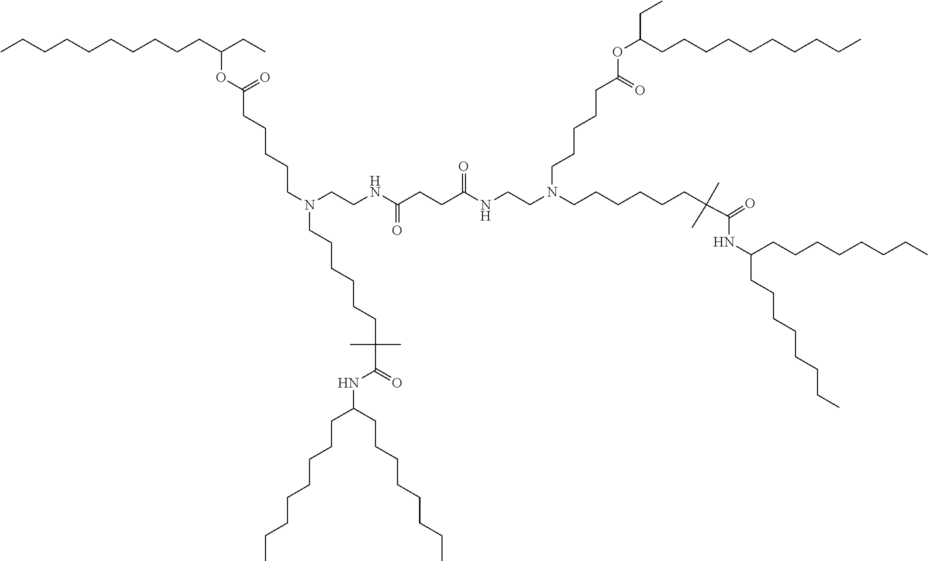 [SMILES: CCCCCCCCCCC(CC)OC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OC(CC)CCCCCCCCCC] | |
| 2561 | 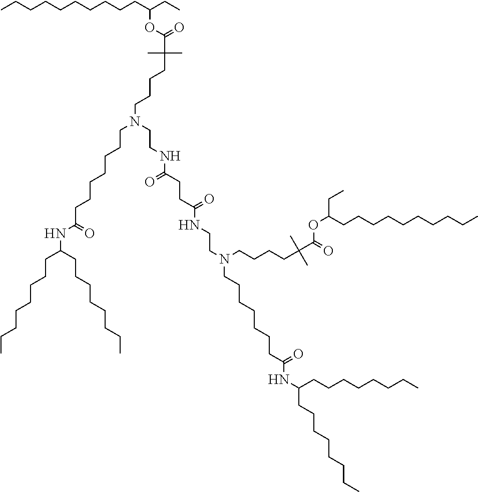 [SMILES: CCCCCCCCCCC(CC)OC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OC(CC)CCCCCCCCCC] | |
| 2563 | 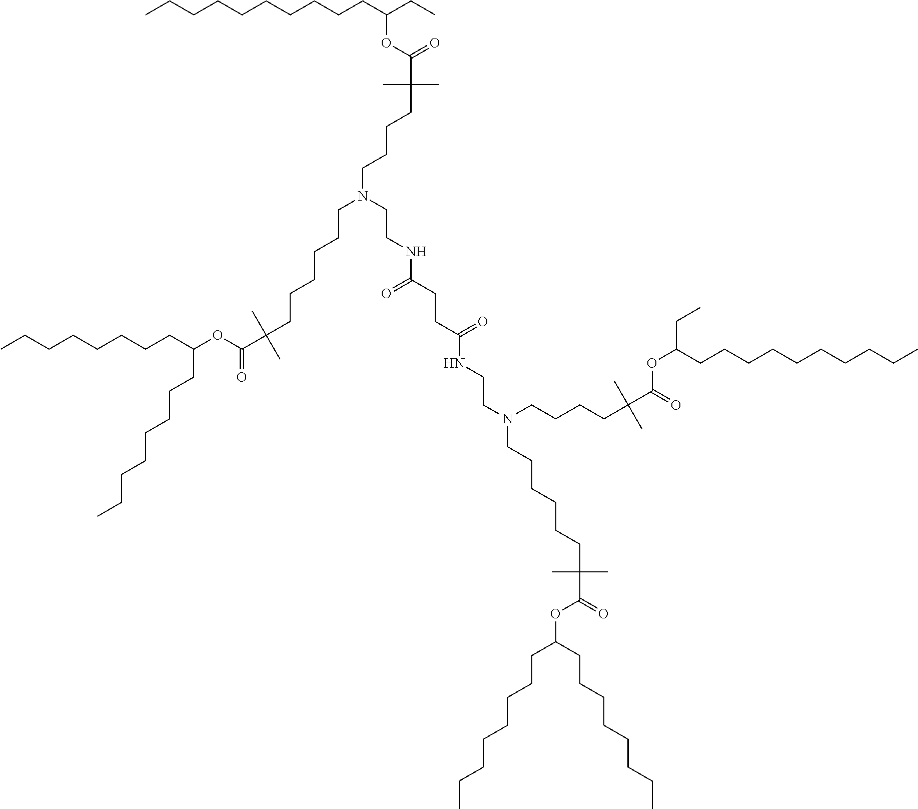 [SMILES: CCCCCCCCCCC(CC)OC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | |
| 2564 | 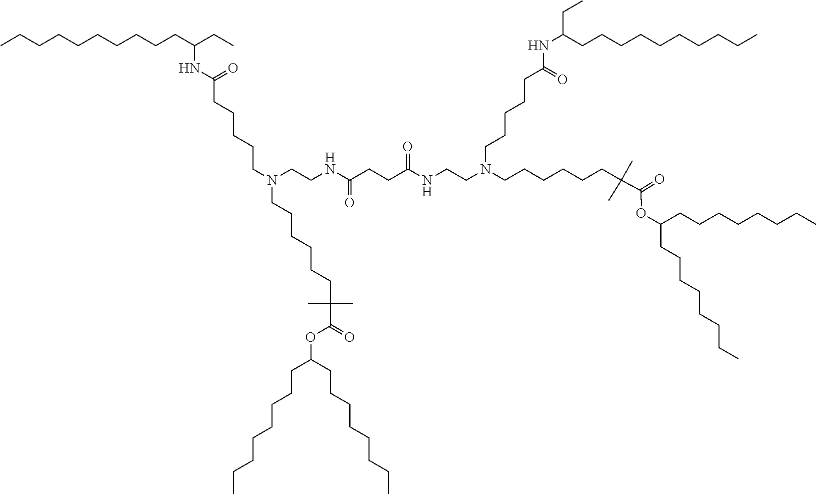 [SMILES: CCCCCCCCCCC(CC)NC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)NC(CC)CCCCCCCCCC] | |
| 2565 | 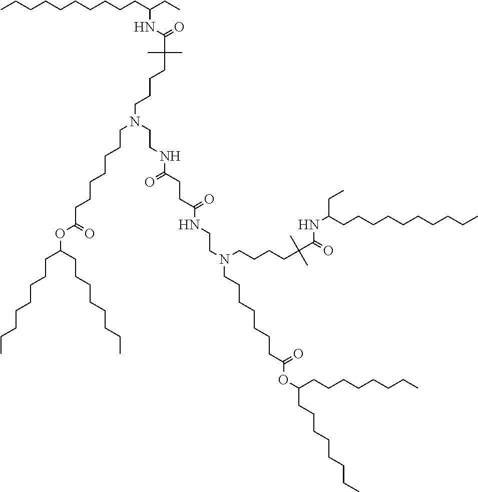 [SMILES: CCCCCCCCCCC(CC)NC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)NC(CC)CCCCCCCCCC] | |
| 2566 | 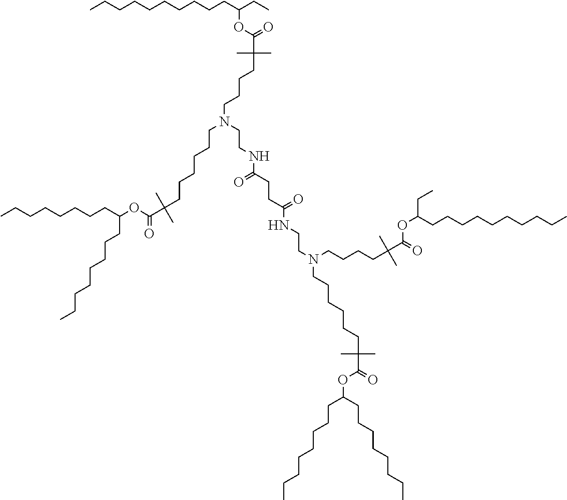 [SMILES: CCCCCCCCCCC(CC)OC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | |
| 2567 | 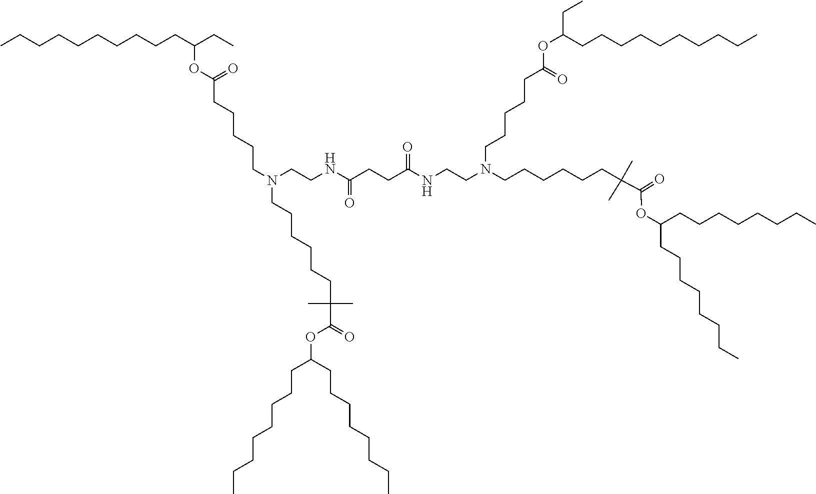 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OC(CC)CCCCCCCCCC] | |
| 2568 | 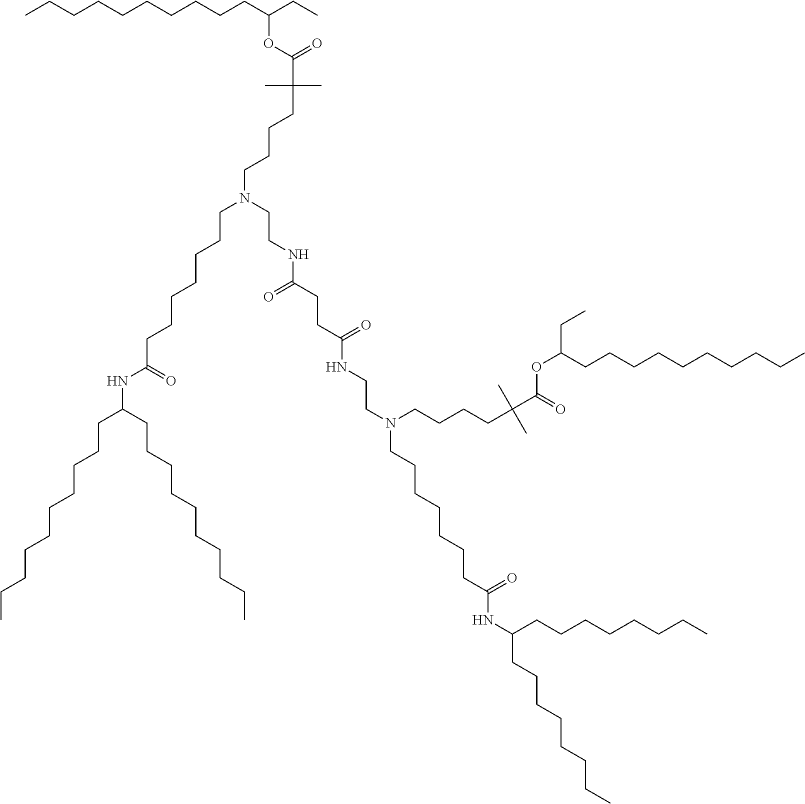 [SMILES: CCCCCCCCCCC(CCCCCCCCCC)NC(=O)CCCCCCCN(CCCCC(C)(C)C(=O)OC(CC)CCCCCCCCCC)CCNC(=O)CCC(=O)NCCN(CCCCCCCC(=O)NC(CCCCCCCC)CCCCCCCC)CCCCC(C)(C)C(=O)OC(CC)CCCCCCCCCC] | |
| 2569 | 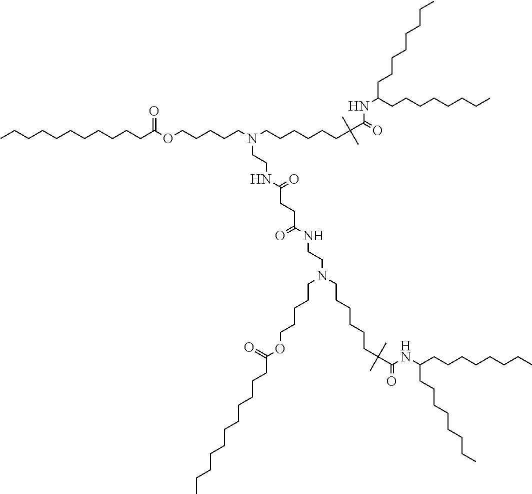 [SMILES: CCCCCCCCCCCC(=O)OCCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCOC(=O)CCCCCCCCCCC] | |
| 2570 | 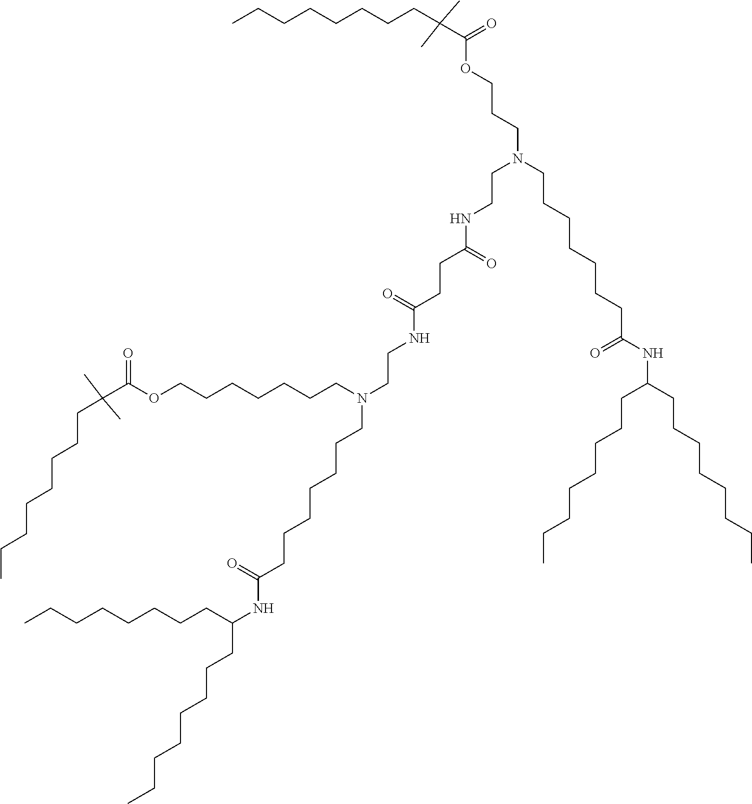 [SMILES: CCCCCCCCC(CCCCCCCC)NC(=O)CCCCCCCN(CCCCCCCOC(=O)C(C)(C)CCCCCCCC)CCNC(=O)CCC(=O)NCCN(CCCCCCCC(=O)NC(CCCCCCCC)CCCCCCCC)CCCOC(=O)C(C)(C)CCCCCCCC] | |
| 2571 | 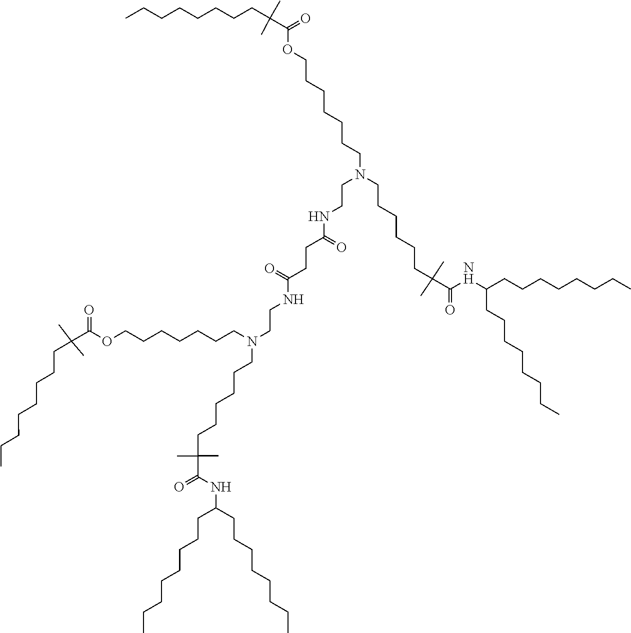 [SMILES: CCCCCCCCC(CCCCCCCC)NC(=O)C(C)(C)CCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)NC(CCCCCCCC)CCCCCCCC] | |
| 2573 | 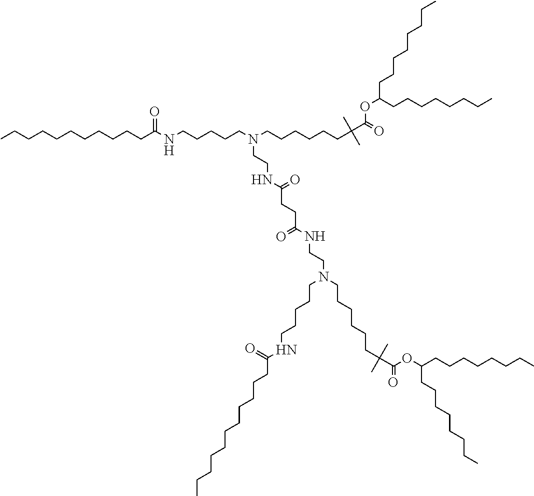 [SMILES: CCCCCCCCCCCC(=O)NCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCNC(=O)CCCCCCCCCCC] | |
| 2574 | 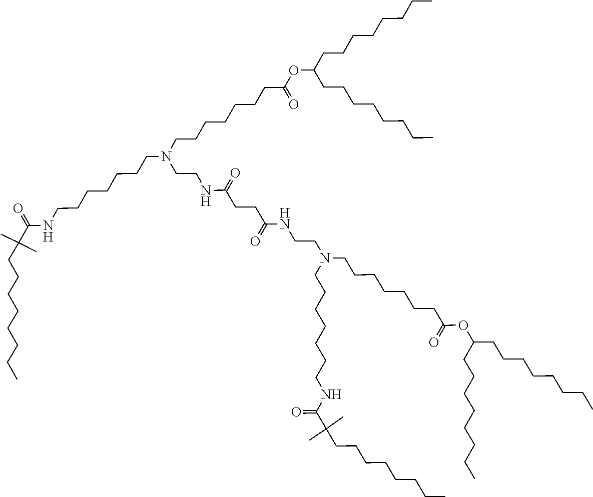 [SMILES: CCCCCCCCC(CCCCCCCC)OC(=O)CCCCCCCN(CCCCCCCNC(=O)C(C)(C)CCCCCCCC)CCNC(=O)CCC(=O)NCCN(CCCCCCCNC(=O)C(C)(C)CCCCCCCC)CCCCCCCC(=O)OC(CCCCCCCC)CCCCCCCC] | |
| 2575 | 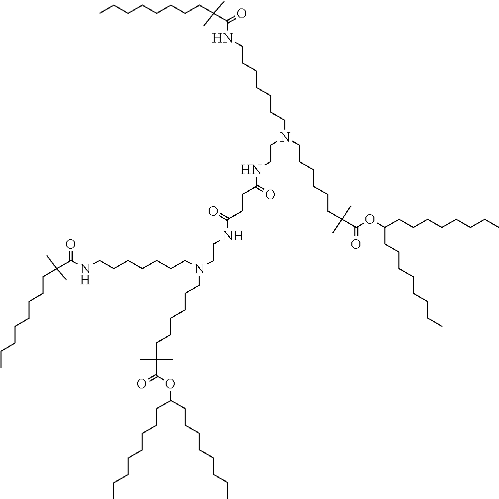 [SMILES: CCCCCCCCC(CCCCCCCC)OC(=O)C(C)(C)CCCCCCN(CCCCCCCNC(=O)C(C)(C)CCCCCCCC)CCNC(=O)CCC(=O)NCCN(CCCCCCCNC(=O)C(C)(C)CCCCCCCC)CCCCCCC(C)(C)C(=O)OC(CCCCCCCC)CCCCCCCC] | |
| 2577 | 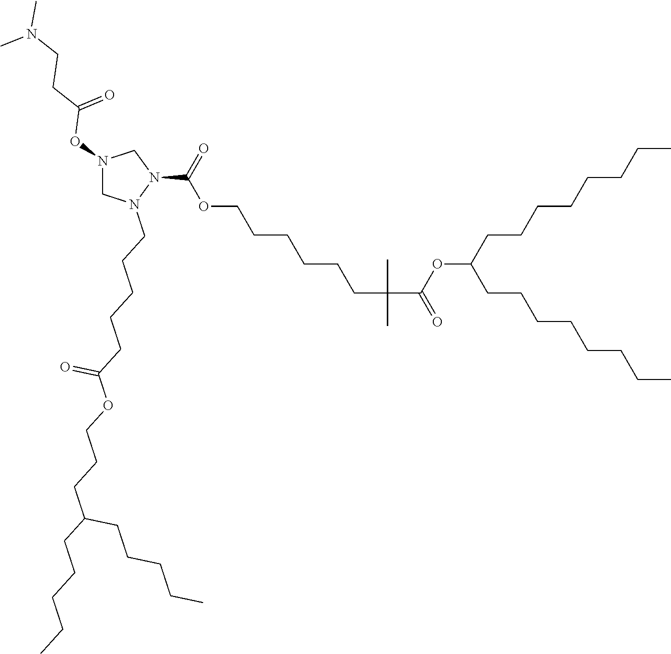 | |
| 2578 | 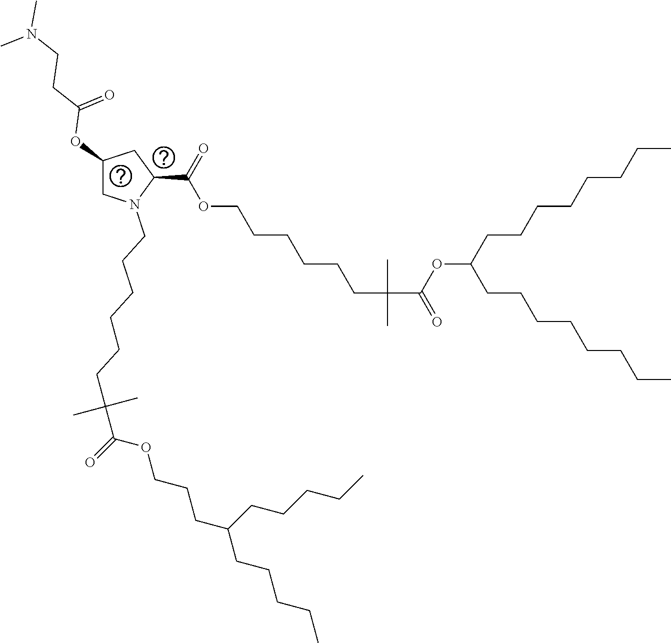 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | |
| 2580 | 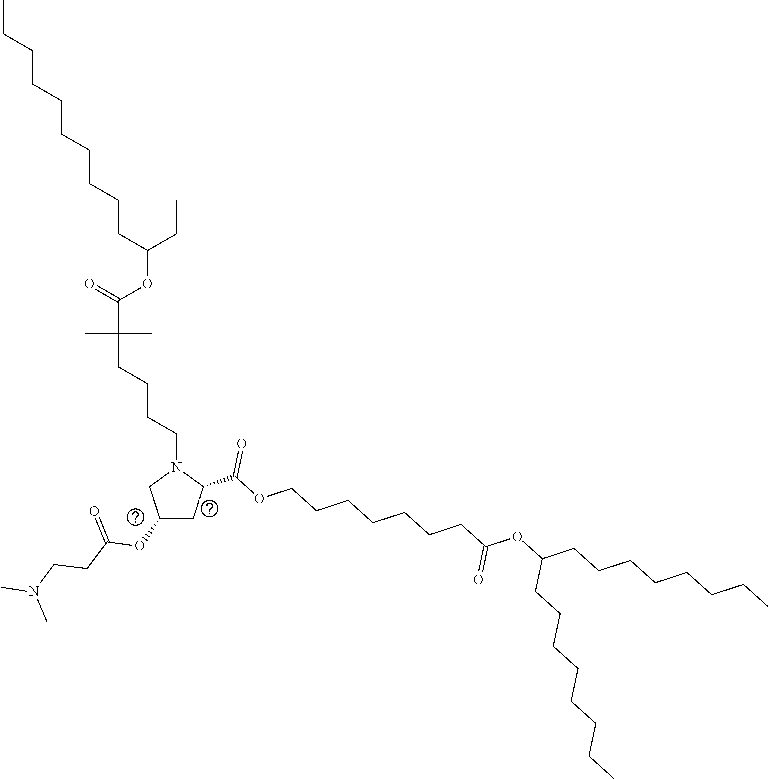 [SMILES: CCCCCCCCCCC(CC)OC(=O)C(C)(C)CCCCN1C[C@@H](OC(=O)CCN(C)C)C[C@H]1C(=O)OCCCCCCCC(=O)OC(CCCCCCCC)CCCCCCCC] | |
| 2581 | 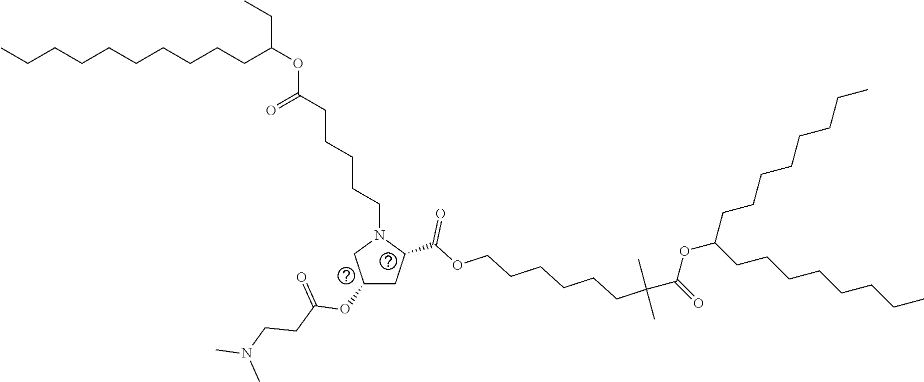 [SMILES: CCCCCCCCCCC(CC)OC(=O)CCCCCN1C[C@@H](OC(=O)CCN(C)C)C[C@H]1C(=O)OCCCCCCC(C)(C)C(=O)OC(CCCCCCCC)CCCCCCCC] | |
| 2582 | 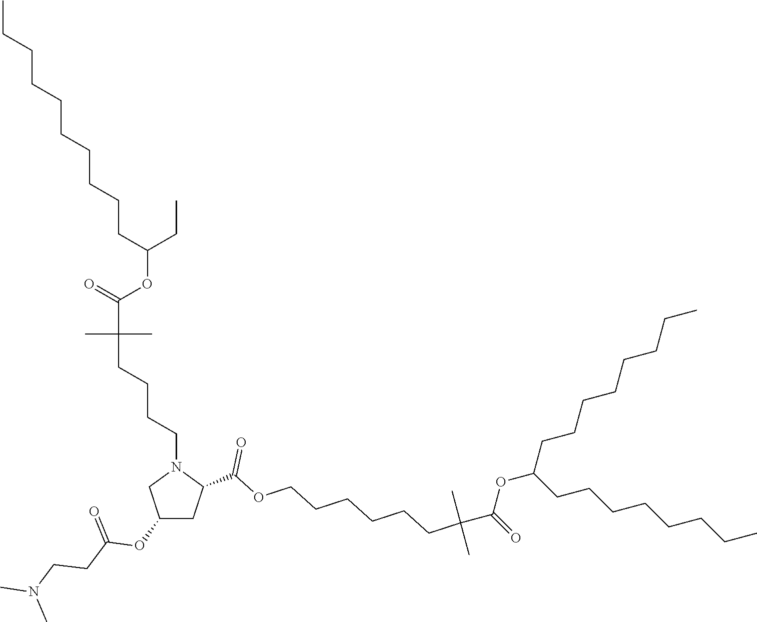 [SMILES: CCCCCCCCCCC(CC)OC(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] | |
| 2583 | 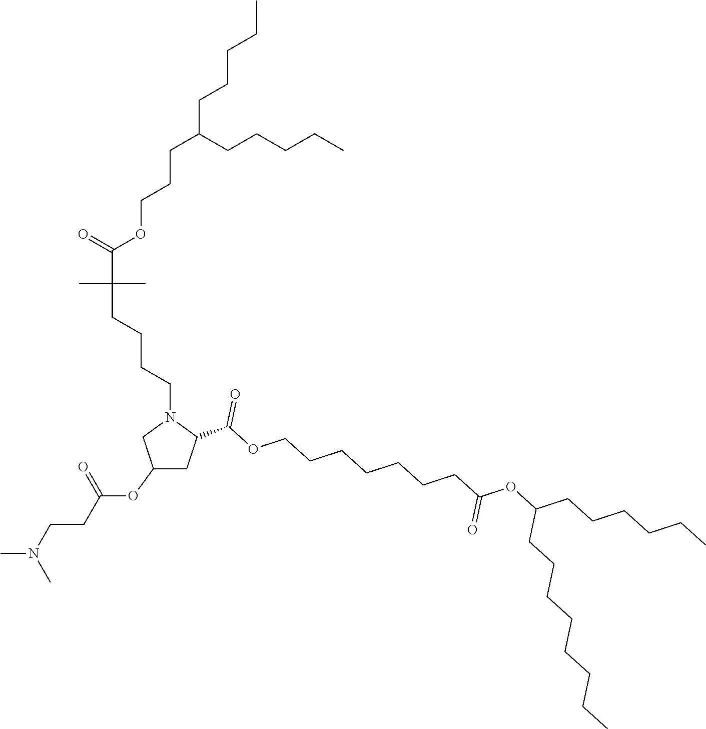 | |
| 2584 | 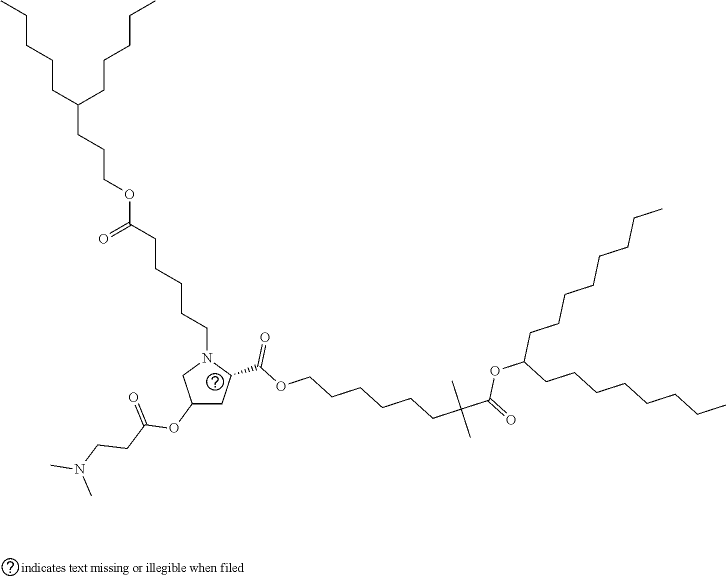 [SMILES: CCCCCCCCC(CCCCCCCC)OC(=O)C(C)(C)CCCCCCOC(=O)[C@@H]1CC(OC(=O)CCN(C)C)CN1CCCCCC(=O)OCCCC(CCCCC)CCCCC] | |
| 2585 | 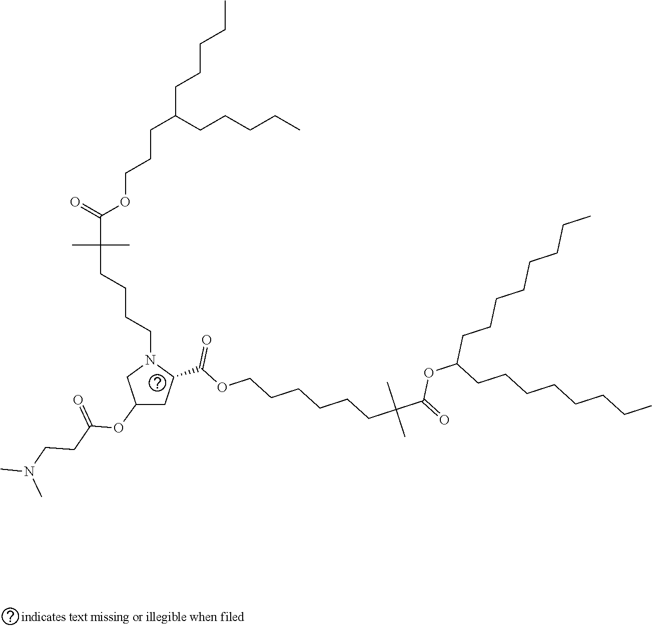 [SMILES: CCCCCCCCC(CCCCCCCC)OC(=O)C(C)(C)CCCCCCOC(=O)[C@@H]1CC(OC(=O)CCN(C)C)CN1CCCCC(C)(C)C(=O)OCCCC(CCCCC)CCCCC] | |
| 2586 | 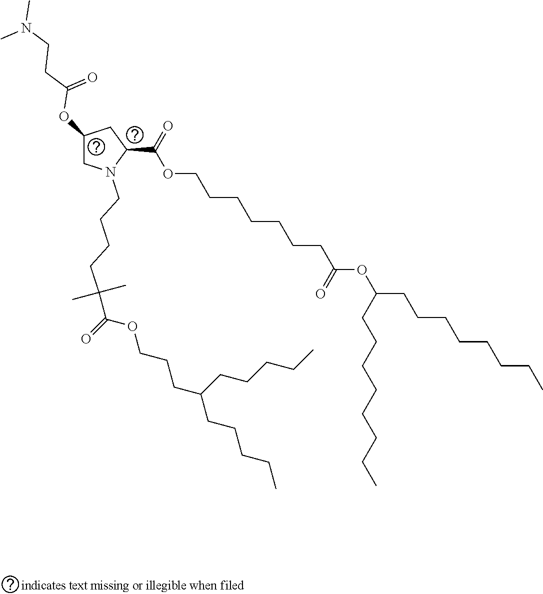 [SMILES: CCCCCCCCC(CCCCCCCC)OC(=O)CCCCCCCOC(=O)[C@@H]1C[C@H](OC(=O)CCN(C)C)CN1CCCCC(C)(C)C(=O)OCCCC(CCCCC)CCCCC] | |
| 2587 | 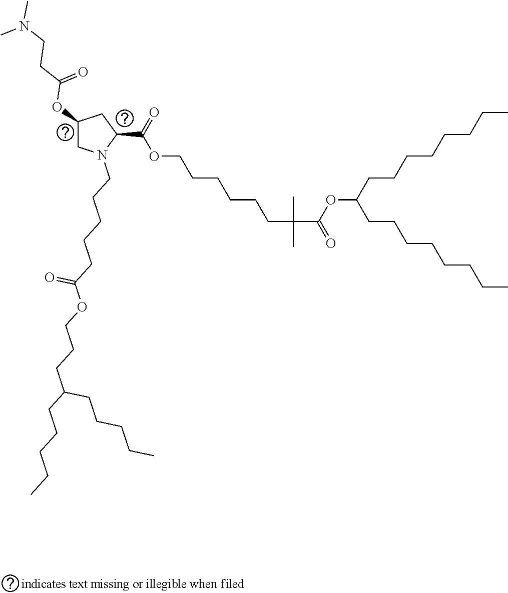 [SMILES: CCCCCCCCC(CCCCCCCC)OC(=O)C(C)(C)CCCCCCOC(=O)[C@@H]1C[C@H](OC(=O)CCN(C)C)CN1CCCCCC(=O)OCCCC(CCCCC)CCCCC] | |
| 2588 | 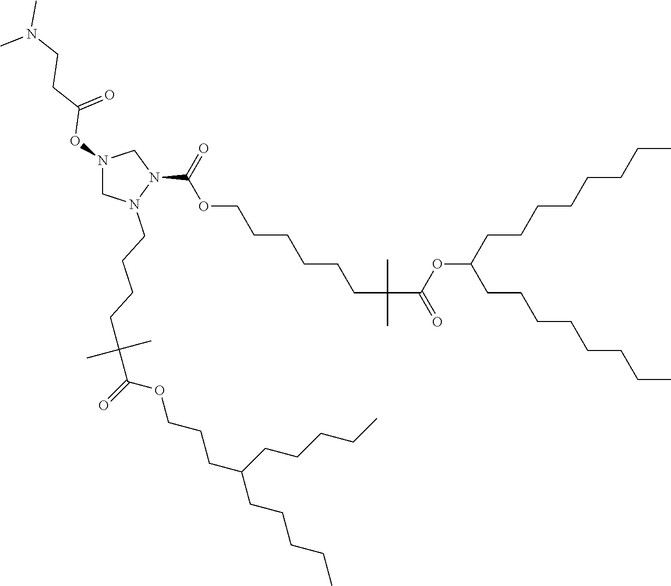 | |
| 2589 | 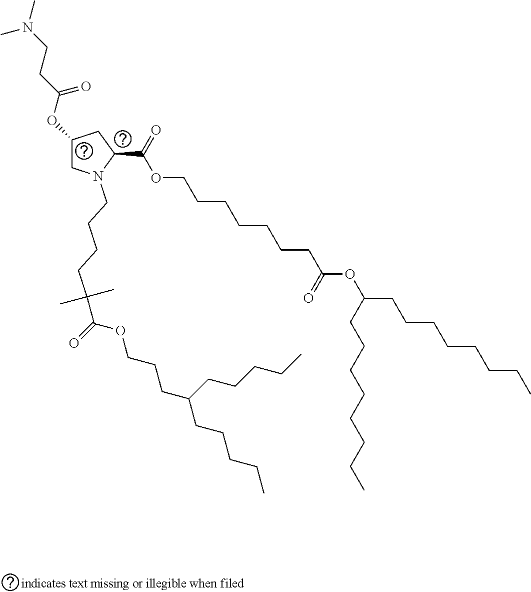 [SMILES: CCCCCCCCC(CCCCCCCC)OC(=O)CCCCCCCOC(=O)[C@@H]1C[C@@H](OC(=O)CCN(C)C)CN1CCCCC(C)(C)C(=O)OCCCC(CCCCC)CCCCC] | |
| 2590 | 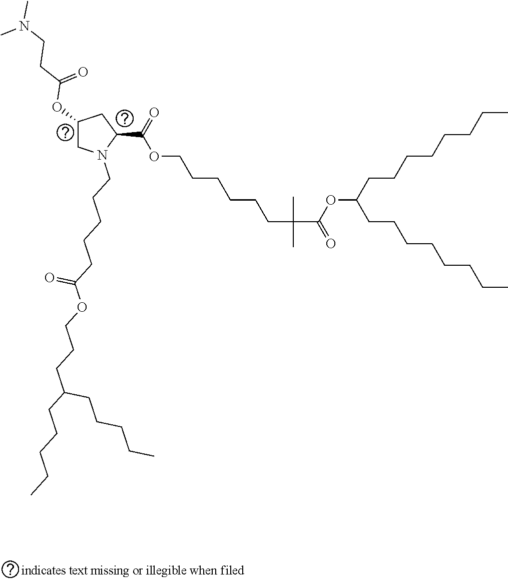 [SMILES: CCCCCCCCC(CCCCCCCC)OC(=O)C(C)(C)CCCCCCOC(=O)[C@@H]1C[C@@H](OC(=O)CCN(C)C)CN1CCCCCC(=O)OCCCC(CCCCC)CCCCC] | |
| 2591 | 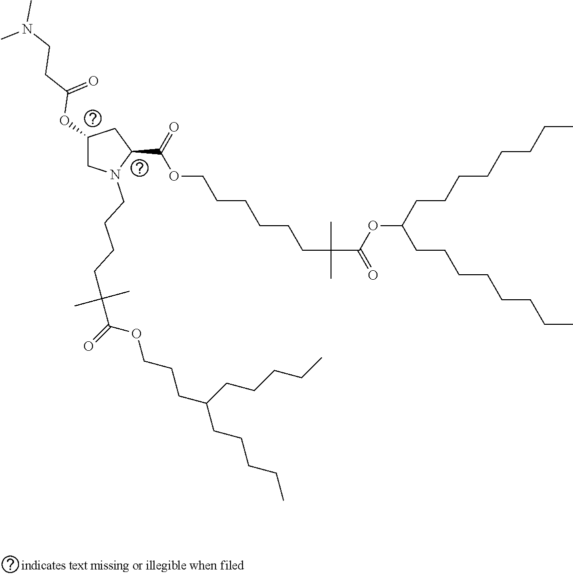 [SMILES: CCCCCCCCC(CCCCCCCC)OC(=O)C(C)(C)CCCCCCOC(=O)[C@@H]1C[C@@H](OC(=O)CCN(C)C)CN1CCCCC(C)(C)C(=O)OCCCC(CCCCC)CCCCC] | |
| 2592 | 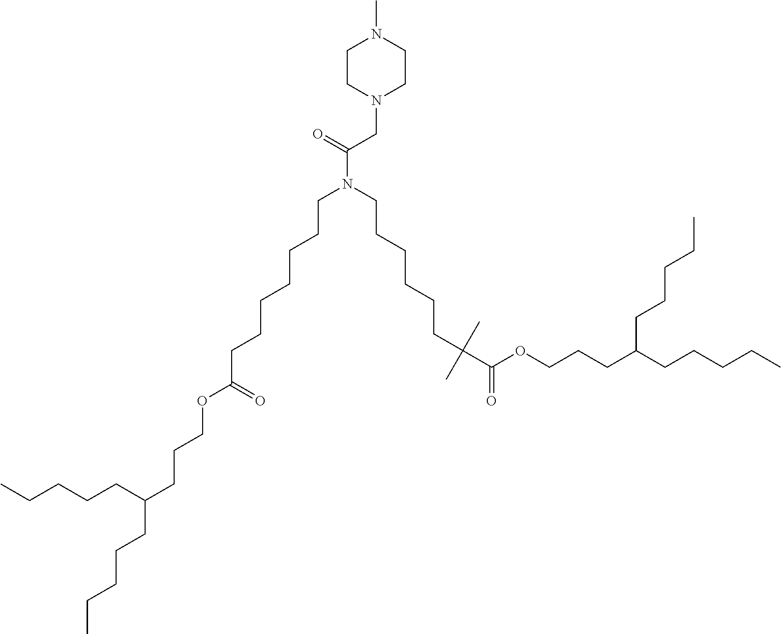 [SMILES: CCCCCC(CCCCC)CCCOC(=O)CCCCCCCN(CCCCCCC(C)(C)C(=O)OCCCC(CCCCC)CCCCC)C(=O)CN1CCN(C)CC1] | |
| 2599 | 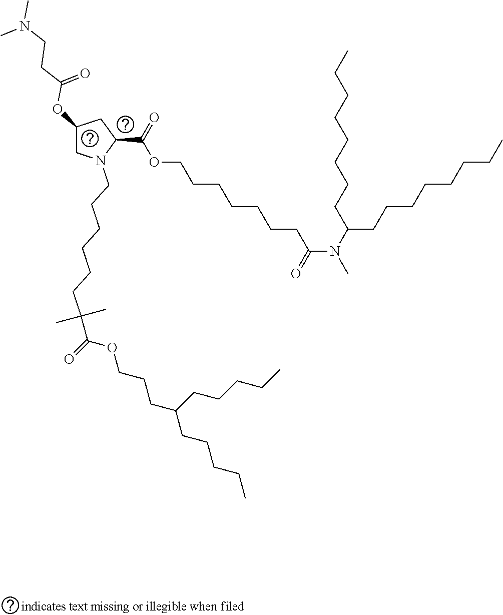 [SMILES: CCCCCCCCC(CCCCCCCC)N(C)C(=O)CCCCCCCOC(=O)[C@@H]1C[C@H](OC(=O)CCN(C)C)CN1CCCCCCC(C)(C)C(=O)OCCCC(CCCCC)CCCCC] | |
| 2600 | 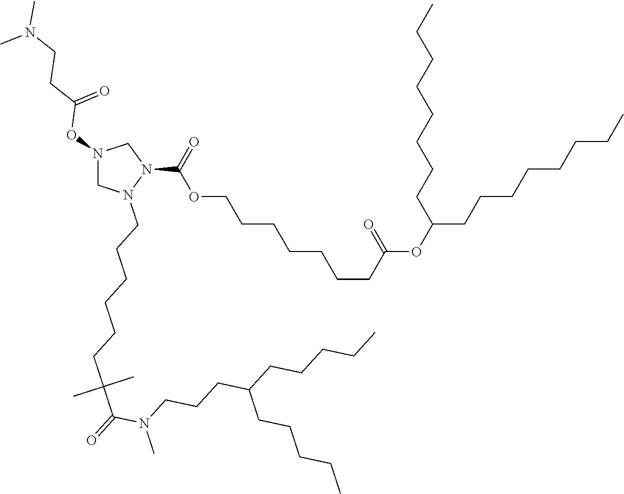 | |
| 2601 | 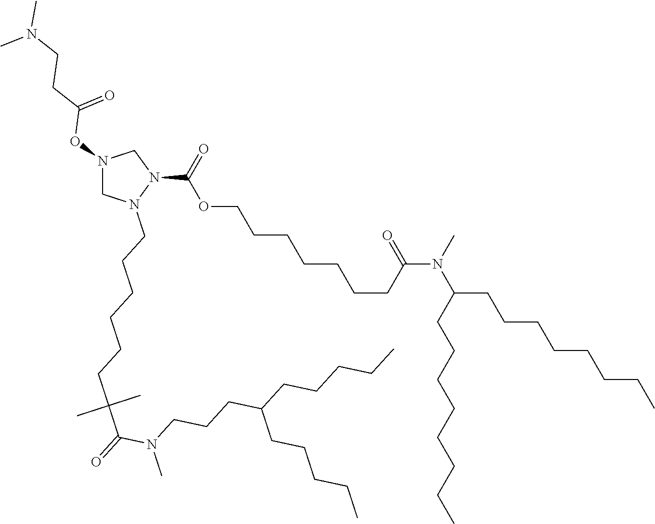 | |
| 2608 | 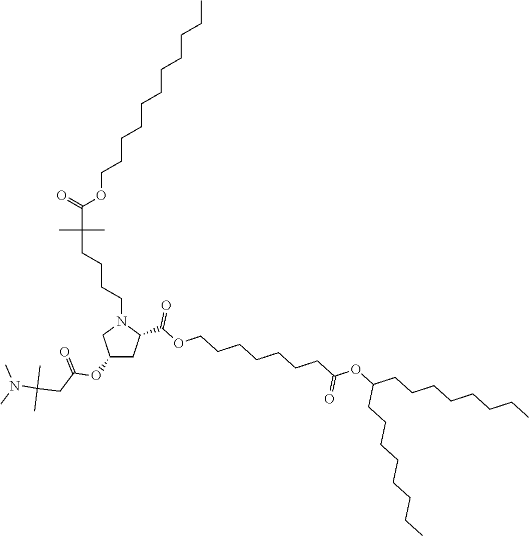 [SMILES: CCCCCCCCCCCOC(=O)C(C)(C)CCCCN1C[C@@H](OC(=O)CC(C)(C)N(C)C)C[C@H]1C(=O)OCCCCCCCC(=O)OC(CCCCCCCC)CCCCCCCC] | |
| 2609 | 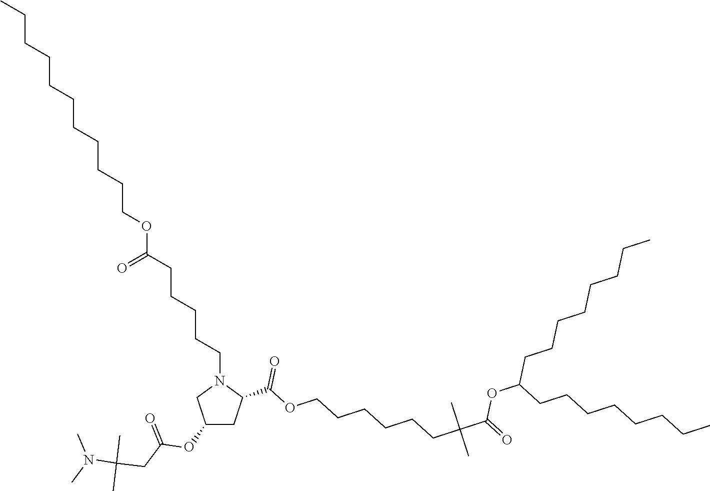 [SMILES: CCCCCCCCCCCOC(=O)CCCCCN1C[C@@H](OC(=O)CC(C)(C)N(C)C)C[C@H]1C(=O)OCCCCCCC(C)(C)C(=O)OC(CCCCCCCC)CCCCCCCC] | |
| 2610 | 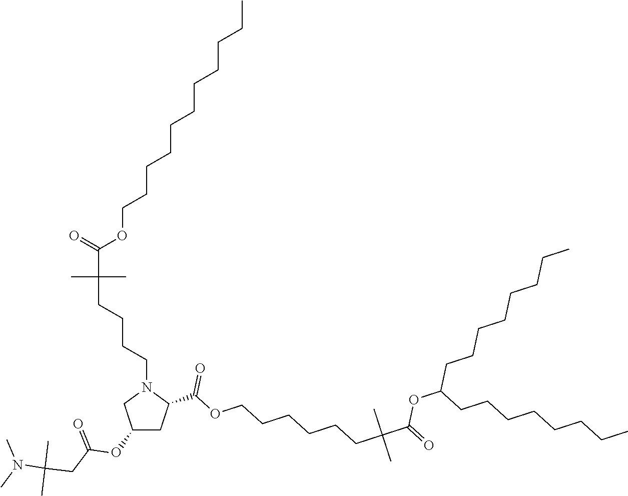 [SMILES: CCCCCCCCCCCOC(=O)C(C)(C)CCCCN1C[C@@H](OC(=O)CC(C)(C)N(C)C)C[C@H]1C(=O)OCCCCCCC(C)(C)C(=O)OC(CCCCCCCC)CCCCCCCC] | |
| 2611 | 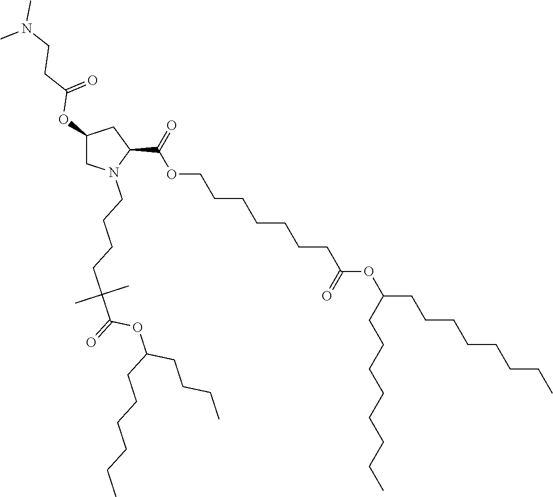 [SMILES: CCCCCCCCC(CCCCCCCC)OC(=O)CCCCCCCOC(=O)[C@@H]1C[C@H](OC(=O)CCN(C)C)CN1CCCCC(C)(C)C(=O)OC(CCCC)CCCCCC] | |
| 2612 | 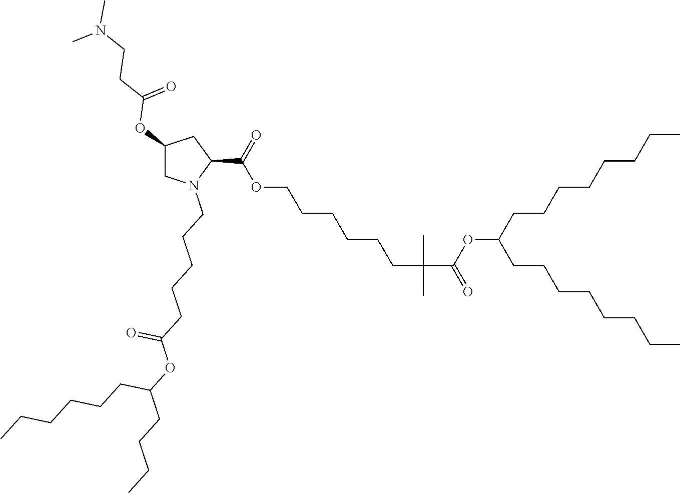 [SMILES: CCCCCCCCC(CCCCCCCC)OC(=O)C(C)(C)CCCCCCOC(=O)[C@@H]1C[C@H](OC(=O)CCN(C)C)CN1CCCCCC(=O)OC(CCCC)CCCCCC] | |
| 2613 | 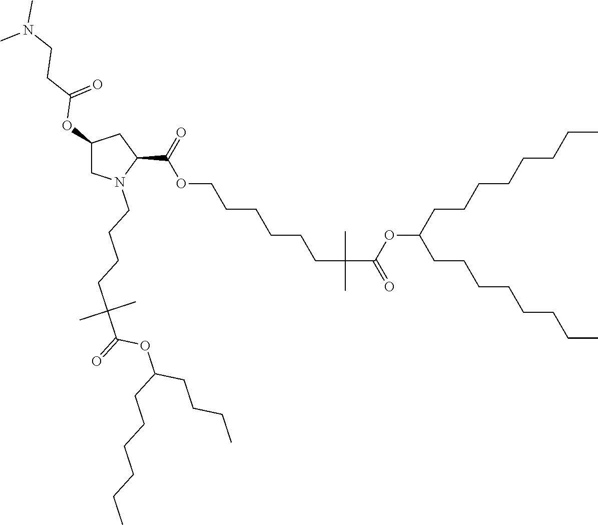 [SMILES: CCCCCCCCC(CCCCCCCC)OC(=O)C(C)(C)CCCCCCOC(=O)[C@@H]1C[C@H](OC(=O)CCN(C)C)CN1CCCCC(C)(C)C(=O)OC(CCCC)CCCCCC] | |
| A1 | 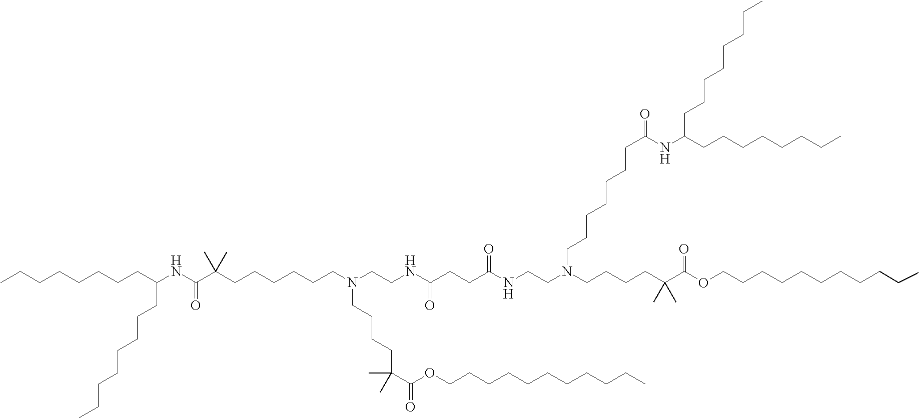 [SMILES: CCCCCCCCCCCOC(=O)C(C)(C)CCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | diundecyl 7-(8- (heptadecan-9- ylamino)-7,7-dimethyl- 8-oxooctyl)-18-(8- (heptadecan-9- ylamino)-8-oxooctyl)- 2,2,23,23-tetramethyl- 11,14-dioxo- 7,10,15,18- tetraazatetracosanedi- oate |
| A2 | 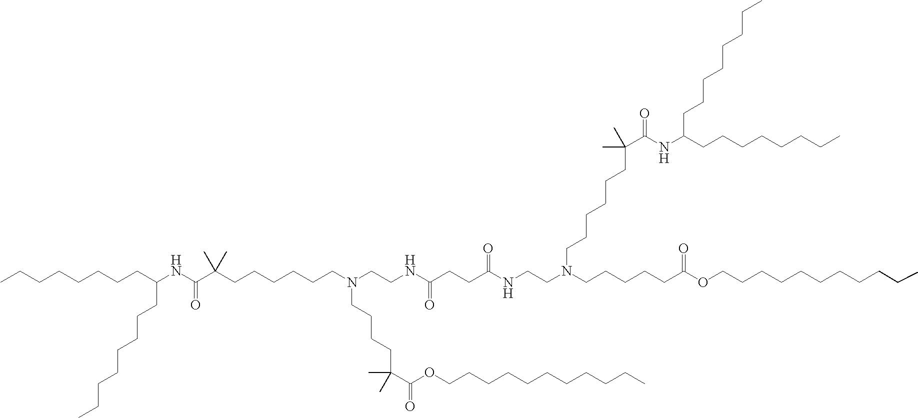 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | diundecyl 7,18-bis(8- (heptadecan-9- ylamino)-7,7-dimethyl- 8-oxooctyl)-2,2- dimethyl-11,14-dioxo- 7,101,15,18- tetraazatetracosanedi- oate |
| A3 | 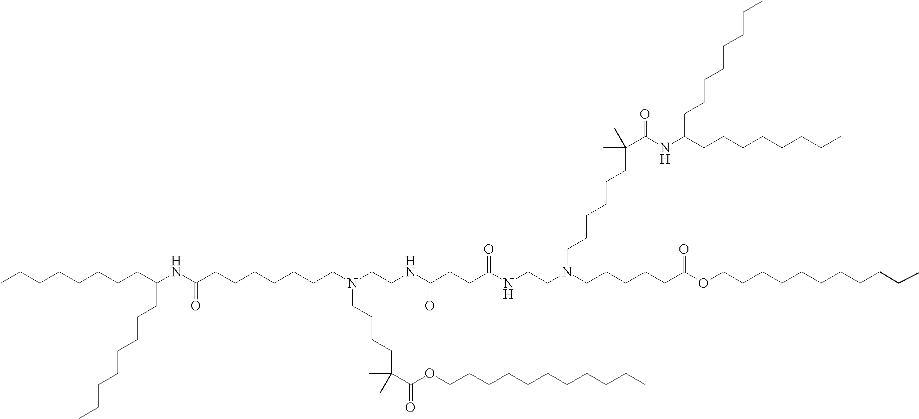 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | diundecyl 18-(8- (heptadecan-9- ylamino)-7,7-dimethyl- 8-oxooctyl)-7-(8- (heptadecan-9- ylamino)-8-oxooctyl)- 2,2-dimethyl-11,14- dioxo-7,10,15,18- tetraazatetracosanedi- oate |
| A4 | 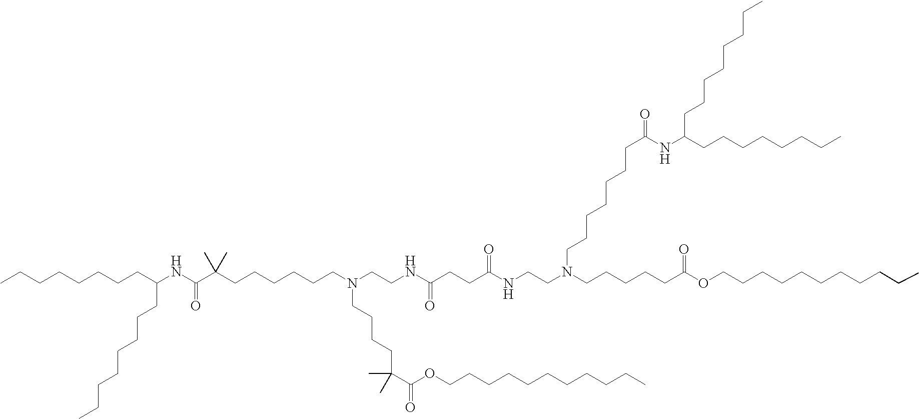 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | diundecyl 8-(8- (heptadecan-9- ylamino)-7,7-dimethyl- 8-oxooctyl)-18-(8- (heptadecan-9- ylamino)-8-oxooctyl)- 2,2-dimethyl-11,14- dioxo-7,10,15,18- tetraazatetracosanedi- oate |
| A5 | 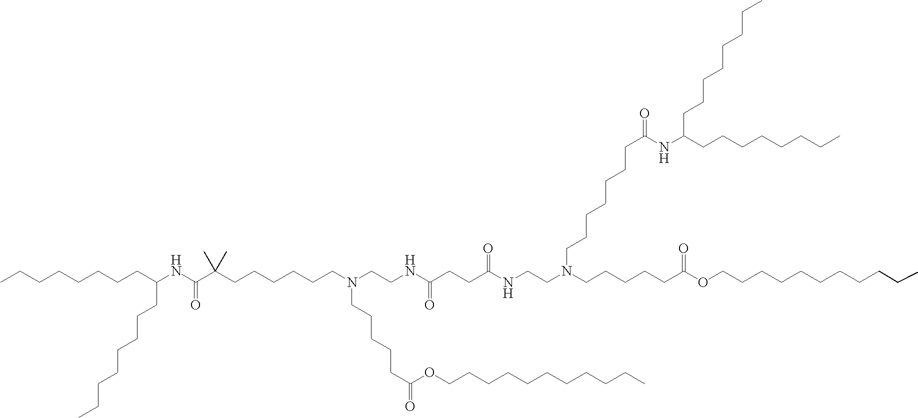 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] | diundecyl 8-(8- (heptadecan-9- ylamino)-7,7-dimethyl- 8-oxooctyl)-18-(8- (heptadecan-9- ylamino)-8-oxooctyl)- 11,14-dioxo- 7,10,15,18- tetraazatetracosanedi- oate |
| A6 | 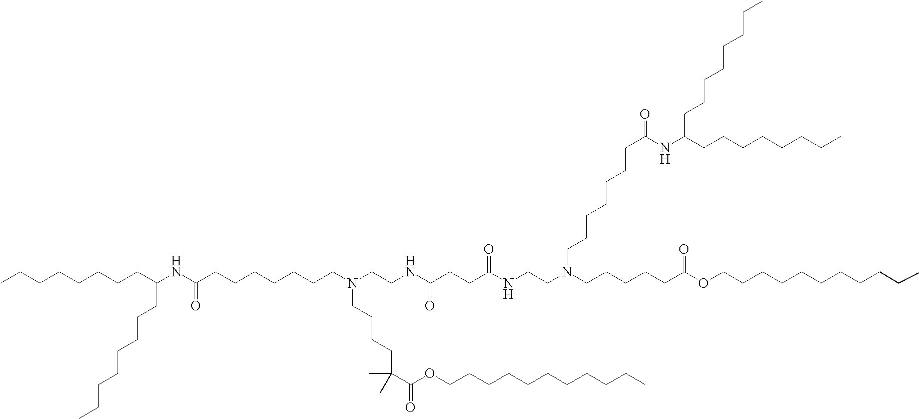 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | diundecyl 7,18-bis(8- (heptadecan-9- ylamino)-8-oxooctyl)- 2,2-dimethyl-11,14- dioxo-7,10,15,18- tetraazatetracosanedi- oate |
| A7 | 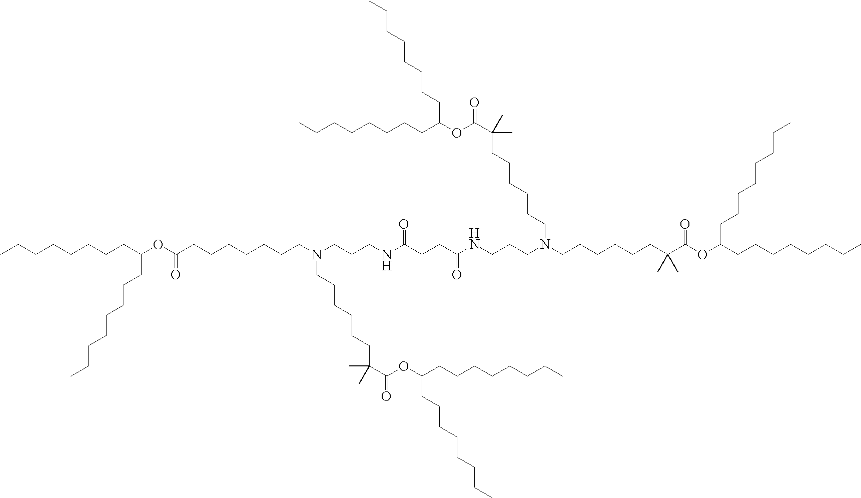 [SMILES: CCCCCCCCC(CCCCCCCC)OC(=O)CCCCCCCN(CCCCCCC(C)(C)C(=O)OC(CCCCCCCC)CCCCCCCC)CCCNC(=O)CCC(=O)NCCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- oxooctyl)-22-(8- (heptadecan-9-yloxy)- 8-oxooctyl)-2,2,29,29- tetramethyl-14,17- dioxo-9,13,18,22- tetraazatriacontanedio- ate |
| A8 | 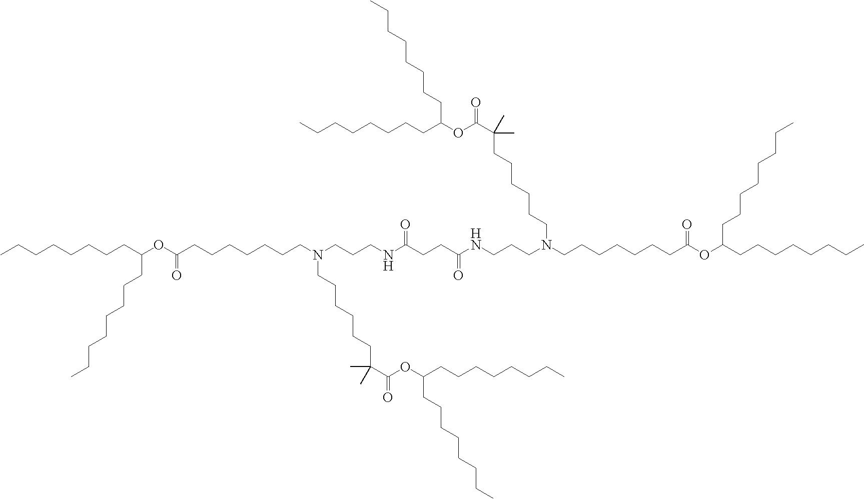 [SMILES: CCCCCCCCC(CCCCCCCC)OC(=O)CCCCCCCN(CCCCCCC(C)(C)C(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,22-bis(8- (heptadecan-9-yloxy)- 8-oxooctyl)-2,2,29,29- tetramethyl-14,17- dioxo-9,13,18,22- tetraazatriacontanedio- ate |
| A9 | 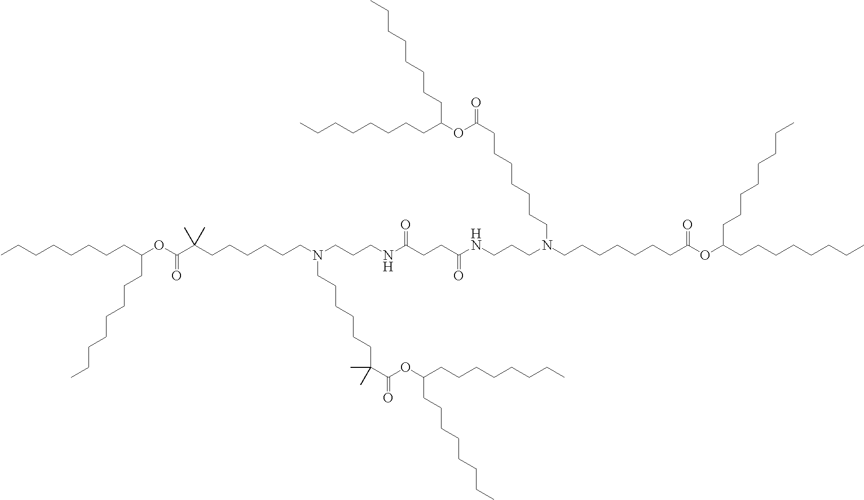 [SMILES: CCCCCCCCC(CCCCCCCC)OC(=O)CCCCCCCN(CCCCCCCC(=O)OC(CCCCCCCC)CCCCCCCC)CCCNC(=O)CCC(=O)NCCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- oxooctyl)-22-(8- (heptadecan-9-yloxy)- 8-oxooctyl)-2,2- dimethyl-14,17-dioxo- 9,13,18,22- tetraazatriacontanedio- ate |
| A10 | 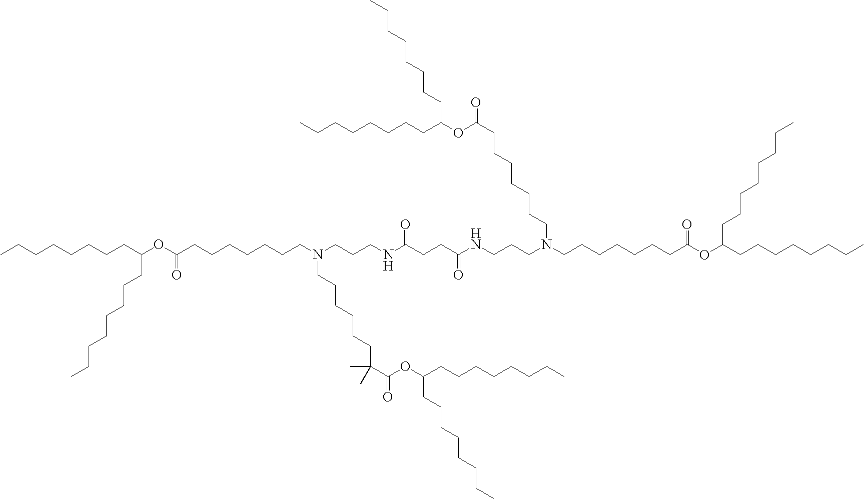 [SMILES: CCCCCCCCC(CCCCCCCC)OC(=O)CCCCCCCN(CCCCCCCC(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,22-bis(8- (heptadecan-9-yloxy)- 8-oxooctyl)-2,2- dimethyl-14,17-dioxo- 9,13,18,22- tetraazatriacontanedio- ate |
| A11 | 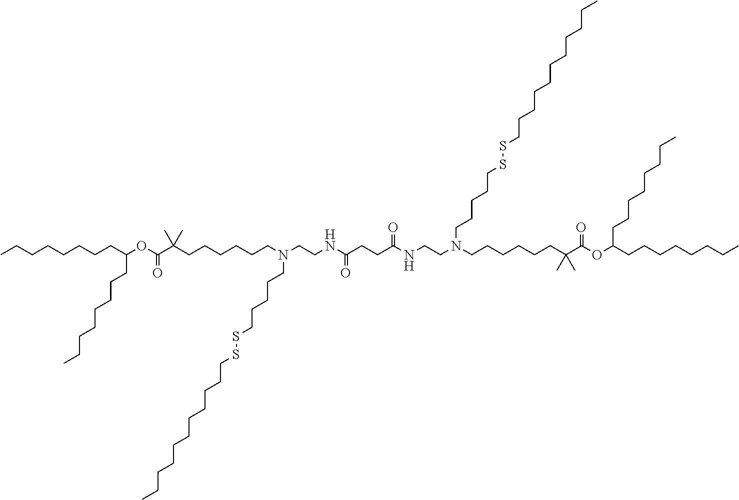 [SMILES: CCCCCCCCCCCSSCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCSSCCCCCCCCCCC] | di(heptadecan-9-yl) 2,2,27,27-tetramethyl- 13,16-dioxo-9,20- bis(5- (8ndecyldisulfaneyl)pen- tyl)-9,12,17,20- tetraazaoctacosanedio- ate |
| A12 | 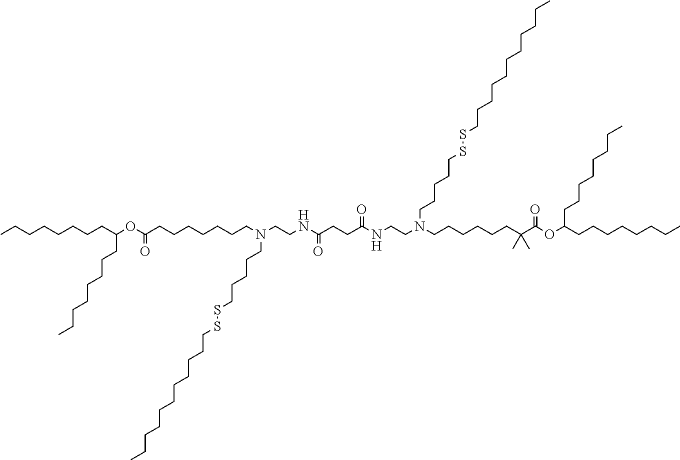 [SMILES: CCCCCCCCCCCSSCCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCSSCCCCCCCCCCC] | di(heptadecan-9-yl) 2,2-dimethyl-13,16- dioxo-9,20-bis(5- (undecyldisulfaneyl)pen- tyl)-9,12,17,20- tetraazaoctacosanedio- ate |
| A13 | 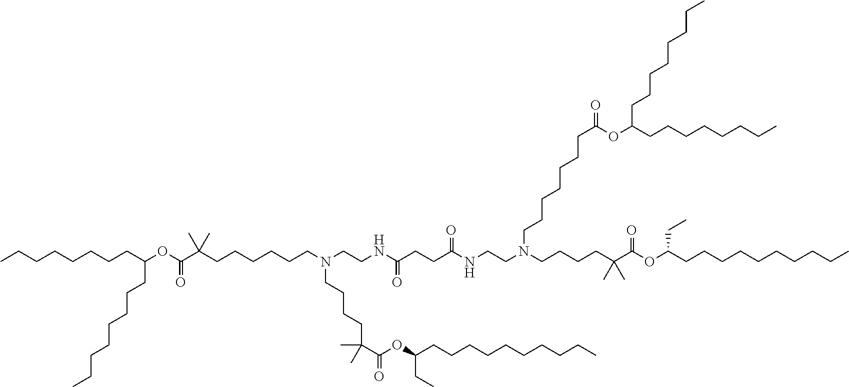 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | di(heptadecan-9-yl) 9,20-bis(5,5-dimethyl- 6-oxo-6-(((S)-tridecan- 3-yl)oxy)hexyl)-2,2- dimethyl-13,16-dioxo- 9,12,17,20- tetraazaoctacosanedio- ate |
| A14 | 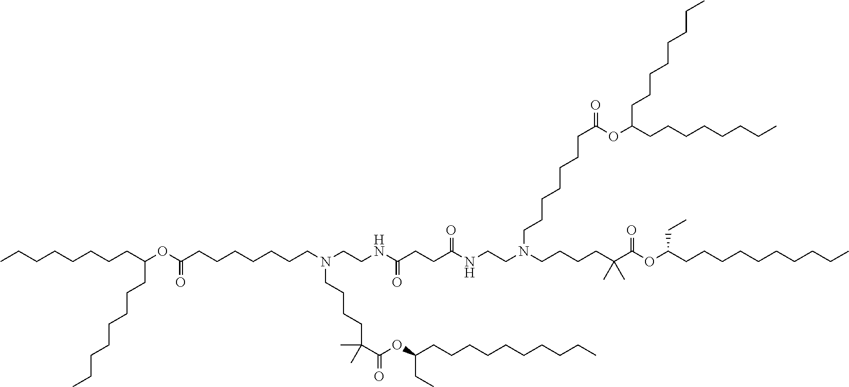 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] | di(heptadecan-9-yl) 9,20-bis(5,5-dimethyl- 6-oxo-6-(((S)-tridecan- 3-yl)oxy)hexyl)-13,16- dioxo-9,12,17,20- tetraazaoctacosanedio- ate |
| A15 | 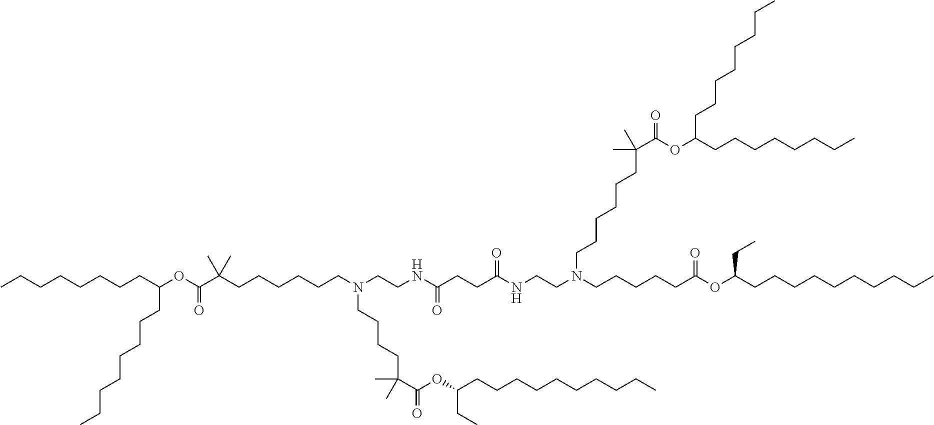 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | di(heptadecna-9-yl) 9- (5,5-dimethyl-6-oxo-6- (((S)-tridecan-3- yl)oxy)hexyl)- 2,2,27,27-tetramethyl- 13,16-dioxo-20-(6- oxo-6-(((S)-tridecan-3- yl)oxy)hexyl)- 9,12,17,20- tetraazaoctacosanedio- ate |
| A16 | 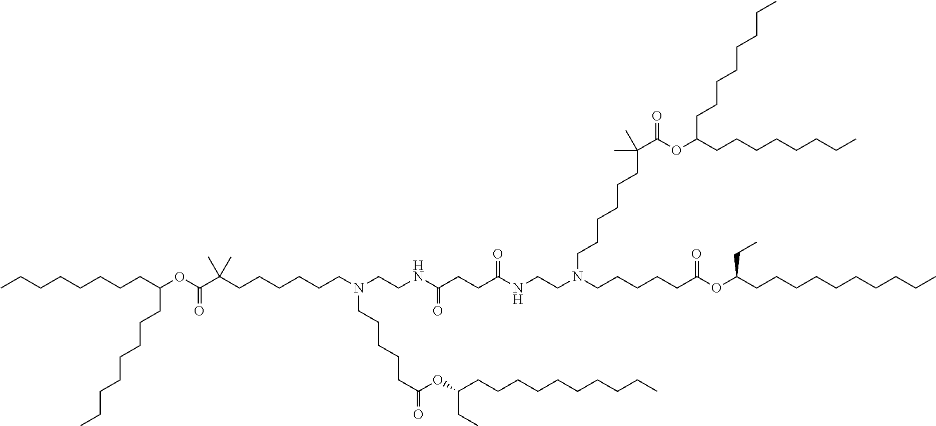 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@@H](CC)CCCCCCCCCC] | di(heptadecan-9-yl) 2,2,27,27-tetramethyl- 13,16-dioxo-9,20- bis(6-oxo-6-(((S)- tridecan-3- yl)oxy)hexyl)- 9,12,17,20- tetraazaoctacosanedio- ate |
| A17 | 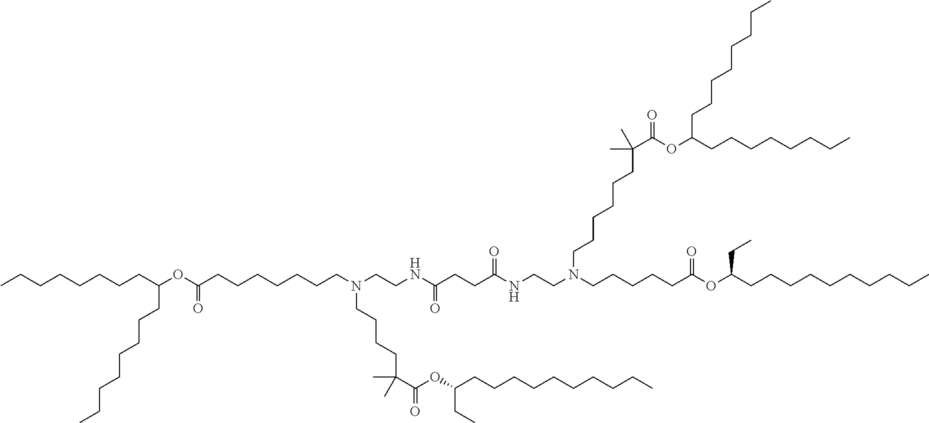 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | di(heptadecan-9-yl) 20-(5,5-dimethyl-6- oxo-6-(((S)-tridecan-3- yl)oxy)hexyl)-2,2- dimethyl-13,16-dioxo- 9-(6-oxo-6-(((S)- tridecan-3- yl)oxy)hexyl)- 9,12,17,20- tetraazaoctacosanedio- ate |
| A18 | 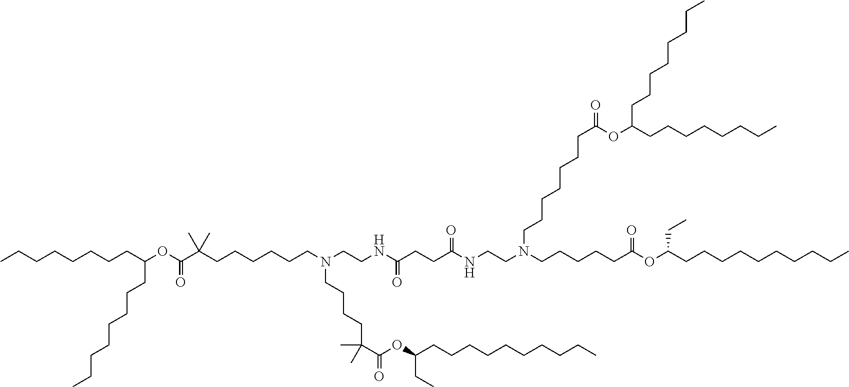 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | di(heptadecan-9-yl) 9- (5,5-dimethyl-6-oxo-6- (((S)-tridecan-3- yl)oxy)hexyl)-2,2- dimethyl-13,16-dioxo- 20-(6-oxo-6-(((S)- tridecan-3- yl)oxy)hexyl)- 9,12,17,20- tetraazaoctacosanedio- ate |
| A19 | 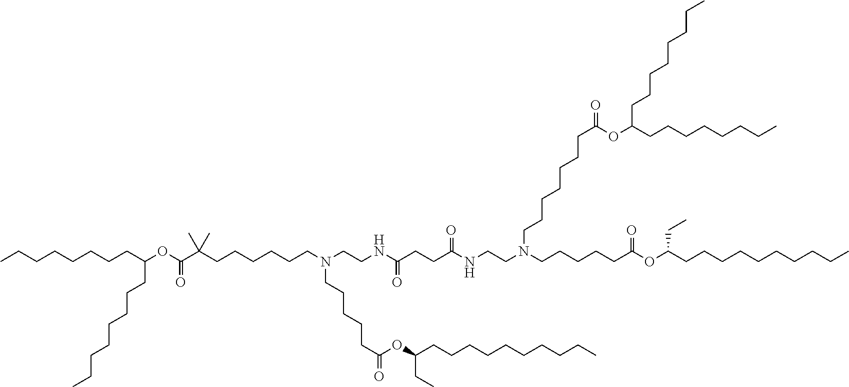 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] | di(heptadecan-9-yl) 2,2-dimethyl-13,16- dioxo-9,20-bis(6-oxo- 6-(((S)-tridecan-3- yl)oxy)hexyl)- 9,12,17,20- tetraazaoctacosanedio- ate |
| A20 | 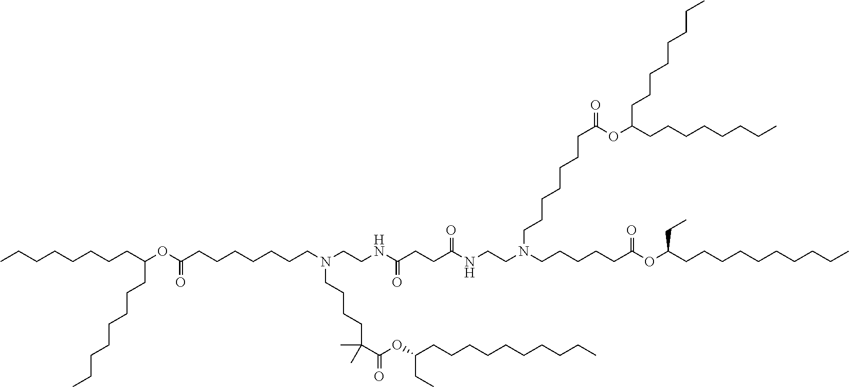 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | di(heptadecan-9-yl) 9- (5,5-dimethyl-6-oxo-6- (((S)-tridecan-3- yl)oxy)hexyl)-13,16- dioxo-20-(6-oxo-6- (((S)-tridecan-3- yl)oxy)hexyl)- 9,12,17,20- tetraazaoctacosanedio- ate |
| A21 | 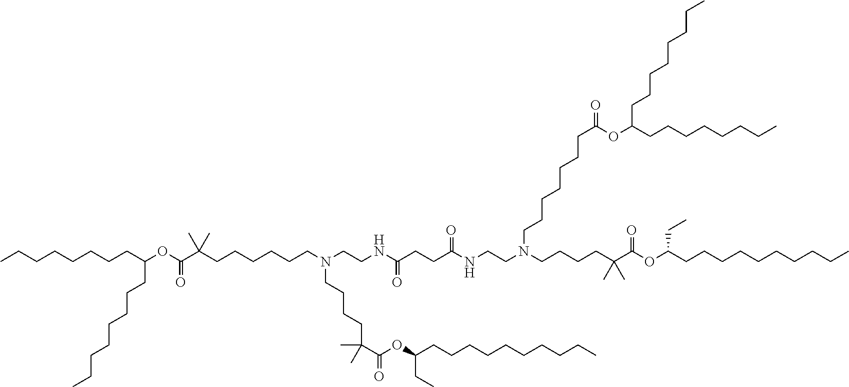 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | di(heptadecan-9-yl) 9,20-bis(5,5-dimethyl- 6-oxo-6-(((R)-tridecan- 3-yl)oxy)hexyl)-2,2- dimethyl-13,16-dioxo- 9,12,17,20- tetraazaoctacosanedio- ate |
| A22 | 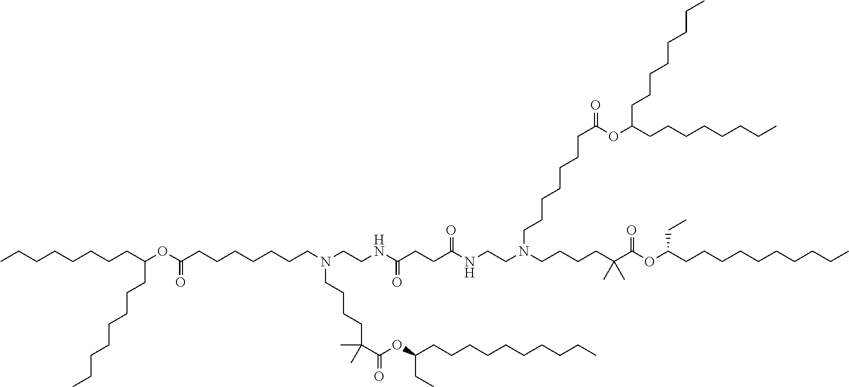 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] | di(heptadecan-9-yl) 9,20-bis(5,5-dimethyl- 6-oxo-6-(((R)-tridecan- 3-yl)oxy)hexyl)-13,16- dioxo-9,12,17,20- tetraazaoctacosanedio- ate |
| A23 | 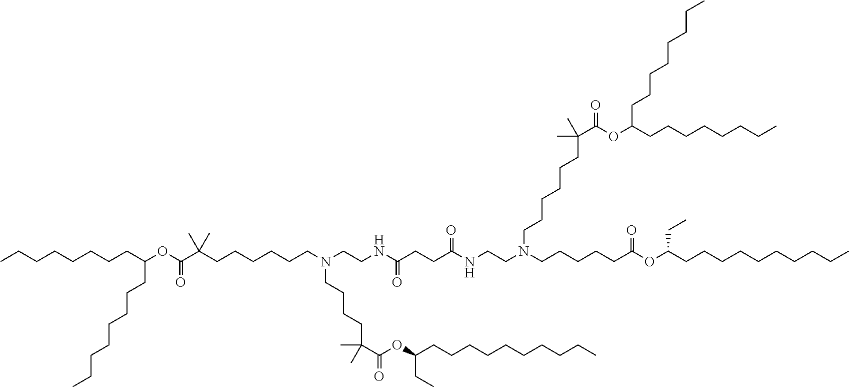 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | di(heptadecan-9-yl) 9- (5,5-dimethyl-6-oxo-6- (((R)-tridecan-3- yl)oxy)hexyl)- 2,2,27,27-tetramethyl- 13,16-dioxo-20-(6- oxo-6-(((R)-tridecan-3- yl)oxy)hexyl)- 9,12,17,20- tetraazaoctacosanedio- ate |
| A24 | 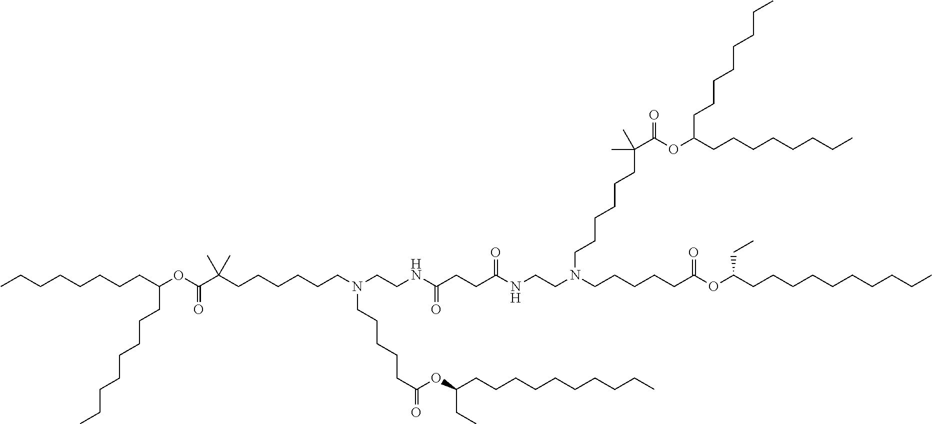 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] | di(heptadecan-9-yl) 2,2,27,27-tetramethyl- 13,16-dioxo-9,20- bis(6-oxo-6-(((R)- tridecan-3- yl)oxy)hexyl)- 9,12,17,20- tetraazaoctacosanedio- ate |
| A25 | 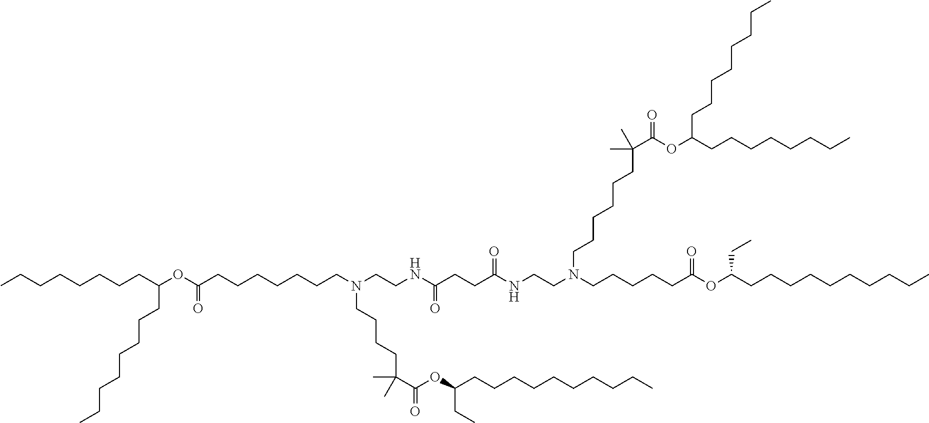 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] | di(heptadecan-9-yl) 20-(5,5-dimethyl-6- oxo-6-(((R)-tridecan-3- yl)oxy)hexyl)-2,2- dimethyl-13,16-dioxo- 9-(6-oxo-6-(((R)- tridecan-3- yl)oxy)hexyl)- 9,12,17,20- tetraazaoctacosanedio- ate |
| A26 | 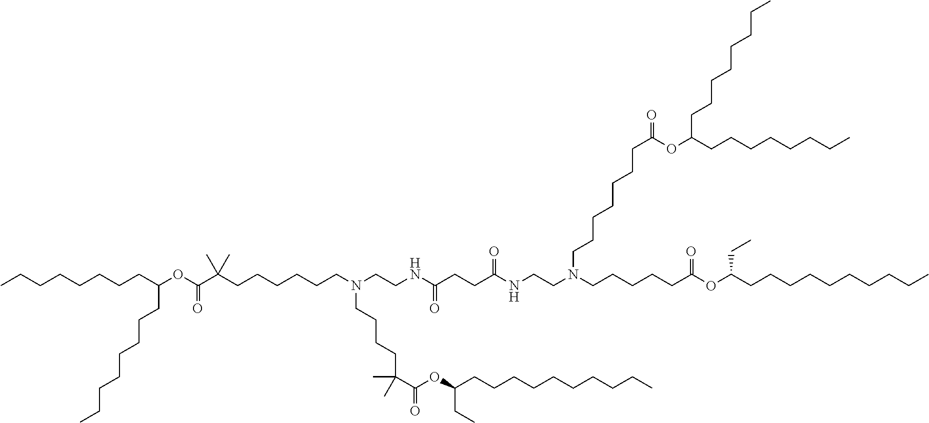 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | di(heptadecan-9-yl) 9- (5,5-dimethyl-6-oxo-6- (((R)-tridecan-3- yl)oxy)hexyl)-2,2- dimethyl-13,16-dioxo- 20-(6-oxo-6-(((R)- tridecan-3- yl)oxy)hexyl)- 9,12,17,20- tetraazaoctacosanedio- ate |
| A27 | 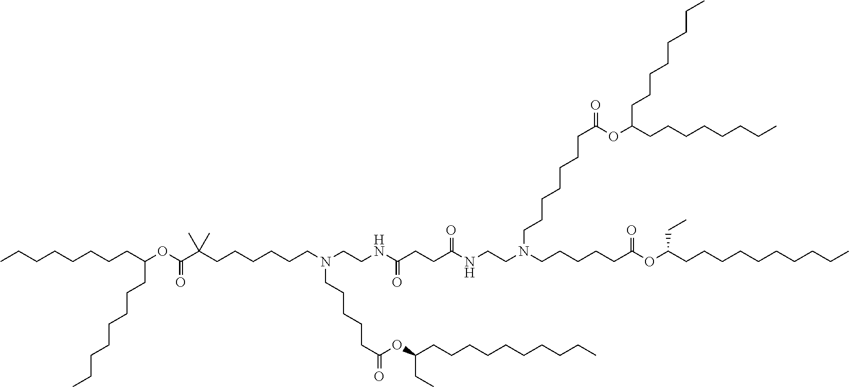 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] | di(heptadecan-9-yl) 2,2-dimethyl-13,16- dioxo-9,20-bis(6-oxo- 6-(((R)-tridecan-3- yl)oxy)hexyl)- 9,12,17,20- tetraazaoctacosanedio- ate |
| A28 | 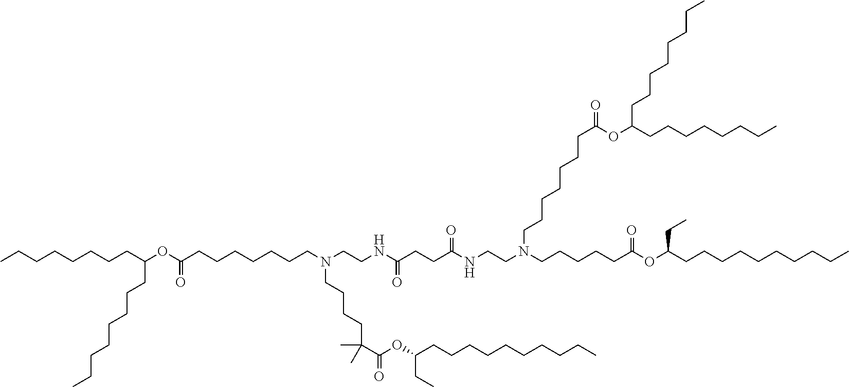 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | di(heptadecan-9-yl) 9- (5,5-dimethyl-6-oxo-6- (((R)-tridecan-3- yl)oxy)hexyl)-13,16- dioxo-20-(6-oxo-6- (((R)-tridecan-3- yl)oxy)hexyl)- 9,12,17,20- tetraazaoctacosanedio- ate |
| A29 | 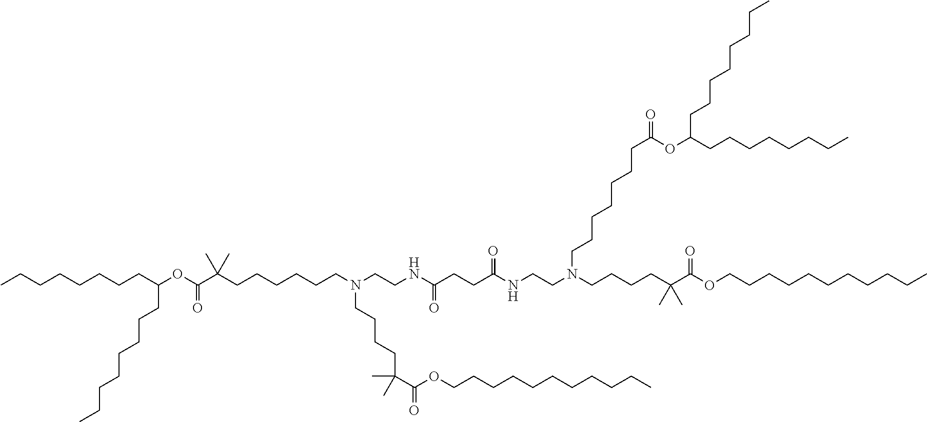 [SMILES: CCCCCCCCCCCOC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,20-bis(5,5-dimethyl- 6-oxo-6- (undecyloxy)hexyl)- 2,2-dimethyl-13,16- dioxo-9,12,17,20- tetraazaoctacosanedio- ate |
| A30 | 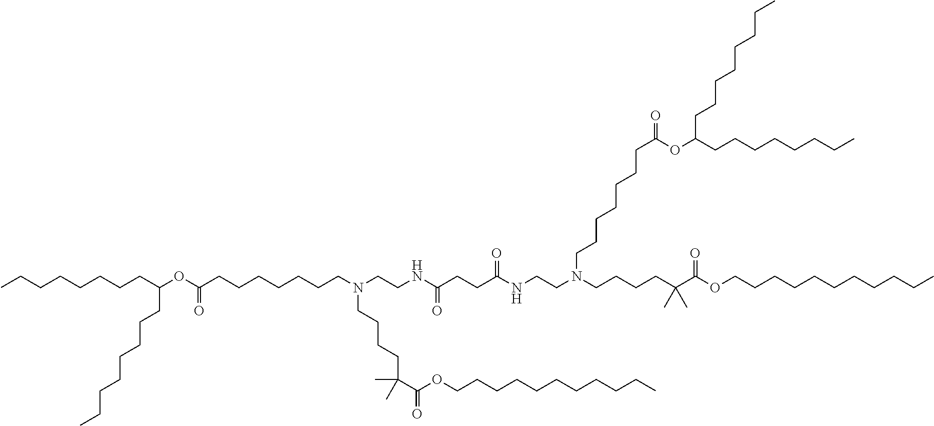 [SMILES: CCCCCCCCCCCOC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | di(heptadecna-9-yl) 9,20-bis(5,5-dimethyl- 6-oxo-6- (undecyloxy)hexyl)- 13,16-dioxo- 9,12,17,20- tetraazaoctacosanedio- ate |
| A31 | 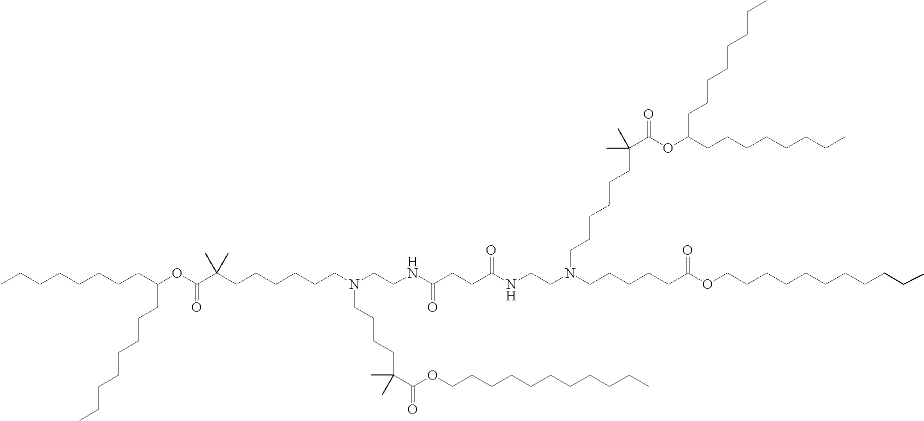 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9- (5,5-dimethyl-6-oxo-6- (undecyloxy)hexyl)- 2,2,27,27-tetramethyl- 13,16-dioxo-20-(6- oxo-6- (undecyloxy)hexyl)- 9,12,17,20- tetraazaoctacosanedio- ate |
| A32 | 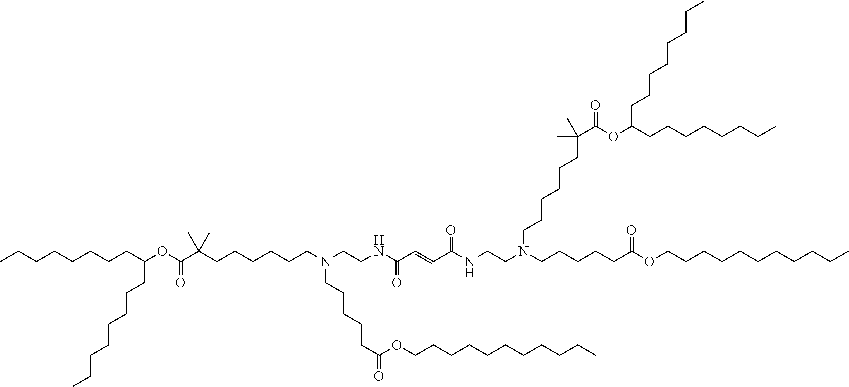 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] | di(heptadecan-9-yl) 2,2,27,27-tetramethyl- 13,16-dioxo-9,20- bis(6-oxo-6- (undecyloxy)hexyl)- 9,12,17,20- tetraazaoctacosanedio- ate |
| A33 | 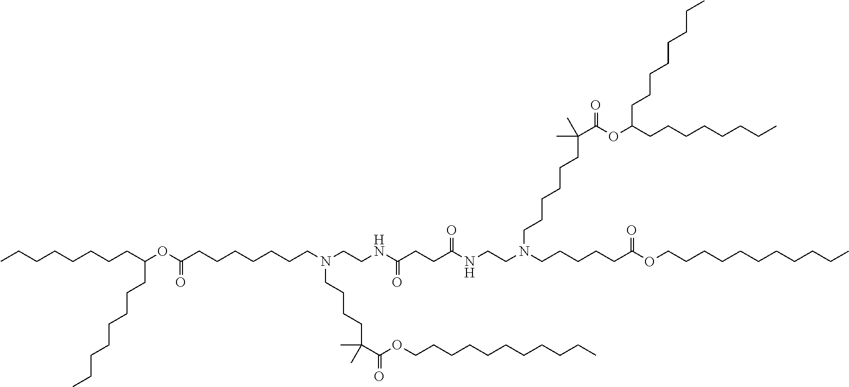 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | di(heptadecan-9-yl) 20-(5,5-dimethyl-6- oxo-6- (undecyloxy)hexyl)- 2,2-dimethyl-13,16- dioxo-9-(6-oxo-6- (undecyloxy)hexyl)- 9,12,17,20- tetraazaoctasanedio- ate |
| A34 | 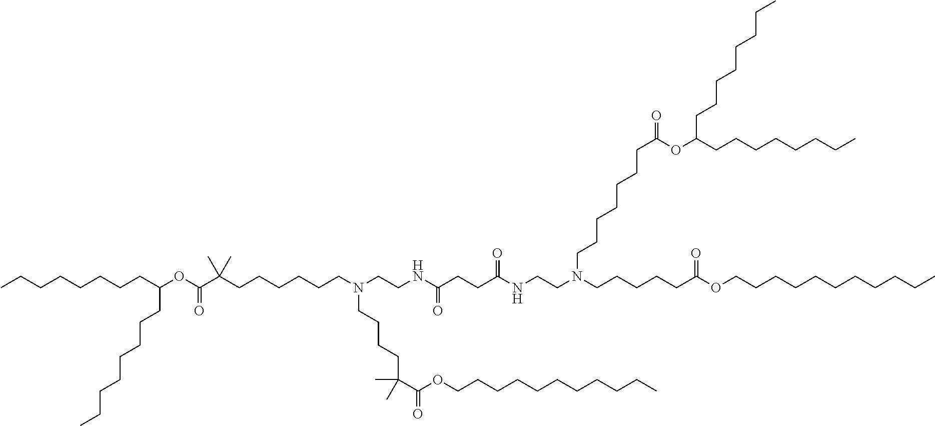 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9- (5,5-dimethyl-6-oxo-6- (undecyloxy)hexyl)- 2,2-dimethyl-13,16- dioxo-20-(6-oxo-6- (undecyloxy)hexyl)- 9,12,17,20- tetraazaoctacosanedio- ate |
| A35 | 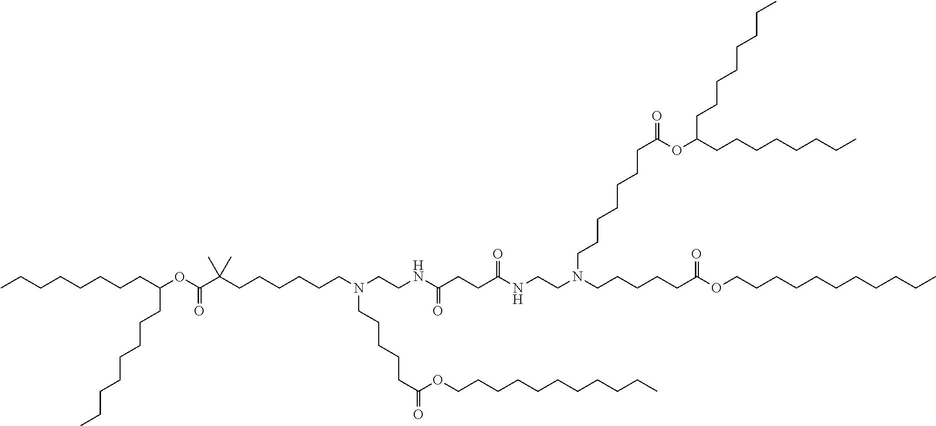 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OCCCCCCCCCCC] | di(heptadecan-9-yl) 2,2-dimethyl-13,16- dioxo-9,20-bis(6-oxo- 6-(undecyloxy)hexyl)- 9,12,17,20- tetraazaoctacosanedio- ate |
| A36 | 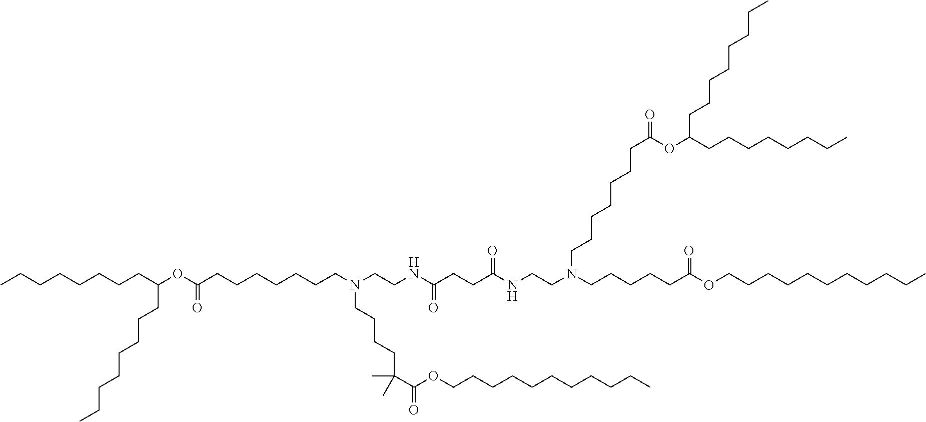 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | di(heptadecan-9-yl) 9- (5,5-dimethyl-6-oxo-6- (undecyloxy)hexyl)- 13,16-dioxo-20-(6- oxo-6- (undecyloxy)hexyl)- 9,12,17,20- tetraazaoctacosanedio- ate |
| A37 | 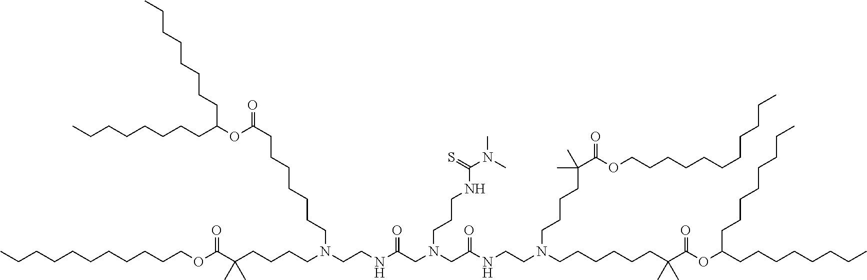 [SMILES: CCCCCCCCCCCOC(=O)C(C)(C)CCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,21-bis(5,5-dimethyl- 6-oxo-6- (undecyloxy)hexyl)-15- (3-(3,3- dimethylthioureido)pro- pyl)-2,2-dimethyl- 13,17-dioxo- 9,12,15,18,21- pentaazanonacosanedi- oate |
| A38 | 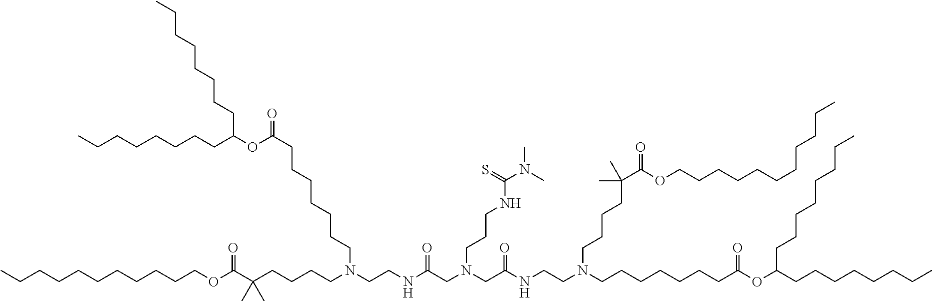 [SMILES: CCCCCCCCCCCOC(=O)C(C)(C)CCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | di(heptadecan-9-yl) 9,21-bis(5,5-dimethyl- 6-oxo-6- (undecyloxy)hexyl)-15- (3-(3,3- dimethylthiourieod)pro- pyl)-13,17-dioxo- 9,12,15,18,21- pentaazanonacosanedi- oate |
| A39 | 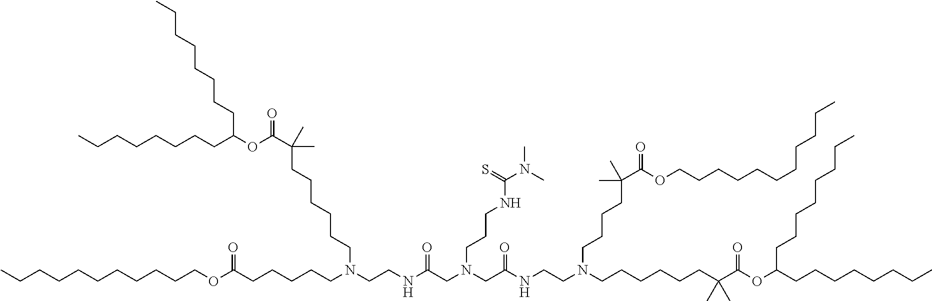 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9- (5,5-dimethyl-6-oxo-6- (undecyloxy)hexyl)-1,5- (3-(3,3- dimethylthioureido)pro- pyl)-2,2,28,28- tetramethyl-13,17- dioxo-21-(6-oxo-6- (undecyloxy)hexyl)- 9,12,15,18,21- pentaazanonacosanedi- oate |
| A40 | 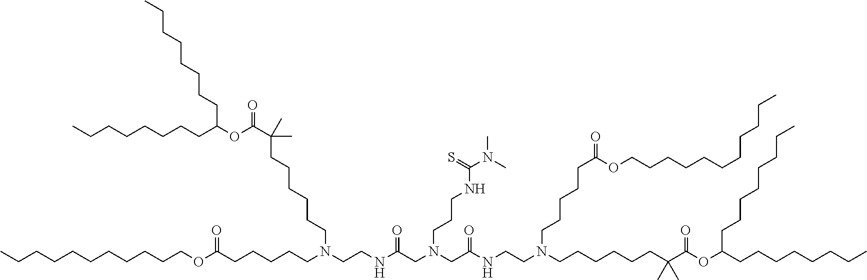 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCCC(=O)OCCCCCCCCCCC] | di(heptadecan-9-yl) 15-(3-(3,3- dimethylthioureido)pro- pyl)-2,2,28,28- tetramethyl-13,17- dioxo-9,21-bis(6-oxo- 6-(undecyloxy)hexyl)- 9,12,15,18,21- pentaaznonacosanedi- oate |
| A41 | 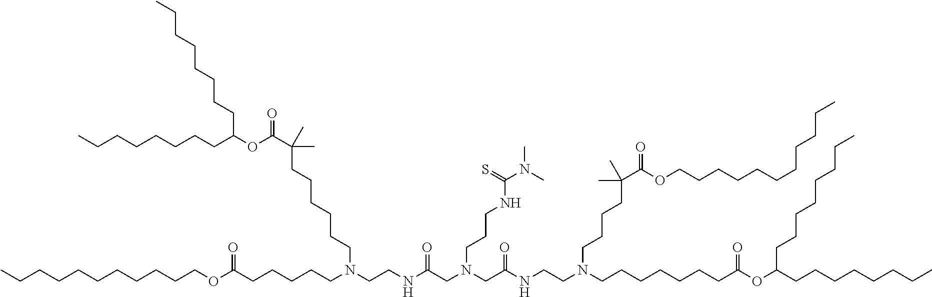 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | di(heptadecan-9-yl) 21-(5,5-dimethyl-6- oxo-6- (undecyloxy)hexyl)-15- (3-(3,3- dimethylthioureido)pro- pyl)-2,2-dimethyl- 13,17-dioxo-9-(6-oxo- 6-(undecyloxy)hexyl)- 9,12,15,18,21- pentaazanonacosanedi- oate |
| A42 | 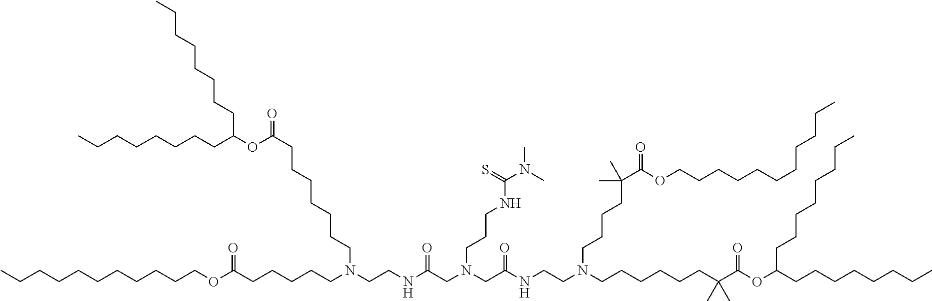 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9- (5,5-dimethyl-6-oxo-6- (undecyloxy)hexyl)-1,5- (3-(3,3- dimethylthioureido)pro- pyl)-2,2-dimethyl- 13,17-dioxo-21-(6- oxo-6- (undecyloxy)hexyl)- 9,12,15,18,21- pentaazanonacosanedi- oate |
| A43 | 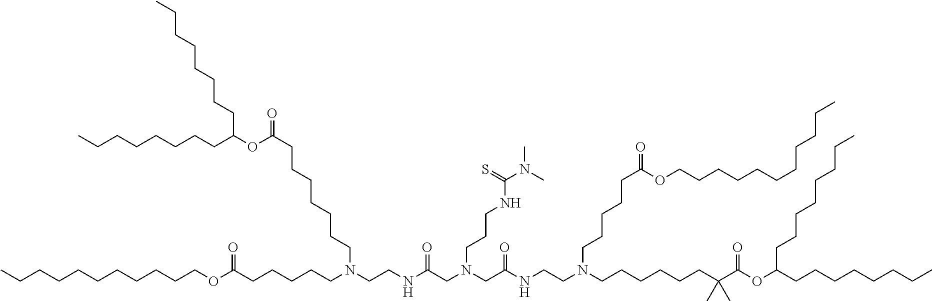 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCCC(=O)OCCCCCCCCCCC] | di(heptadecan-9-yl) 15-(3-(3,3- dimethylthioureido)pro- pyl)-2,2-dimethyl- 13,17-dioxo-9,21- bis(6-oxo-6- (undecyloxy)hexyl)- 9,12,15,8,21- pentaazanonacosanedi- oate |
| A44 | 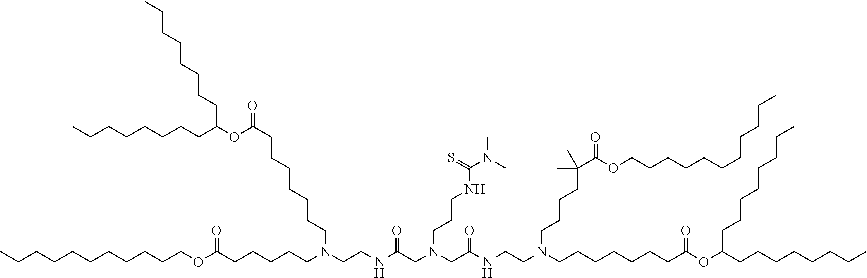 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | di(heptadecan-9-yl) 9- (5,5-dimethyl-6-oxo-6- (undecyloxy)hexyl)-15- (3-(3,3- dimethylthioureido)pro- pyl)-13,17-dioxo-21- (6-oxo-6- (undecyloxy)hexyl)- 9,12,15,18,21- pentaazanonacosanedi- oate |
| A45 | 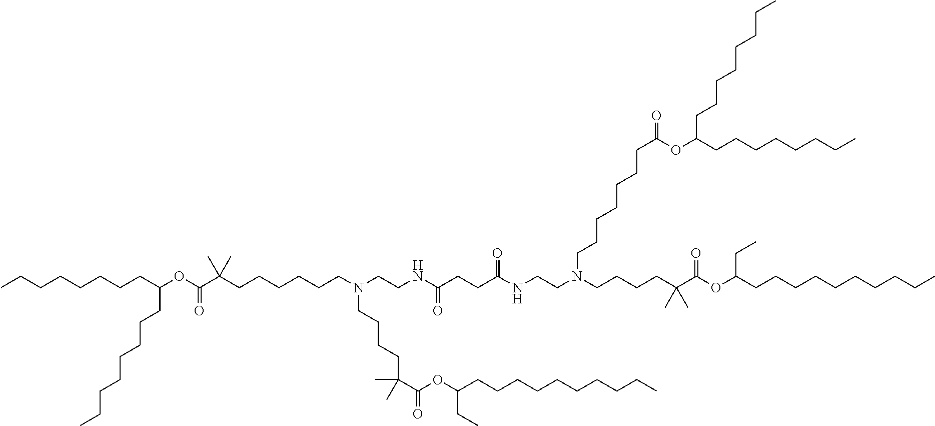 [SMILES: CCCCCCCCCCC(CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | di(heptadecan-9-yl) 9,20-bis(5,5-dimethyl- 6-oxo-6-(tridecan-3- yloxy)hexyl)-2,2- dimethyl-13,16-dioxo- 9,12,17,20- tetraazaoctacosanedio- ate |
| A46 | 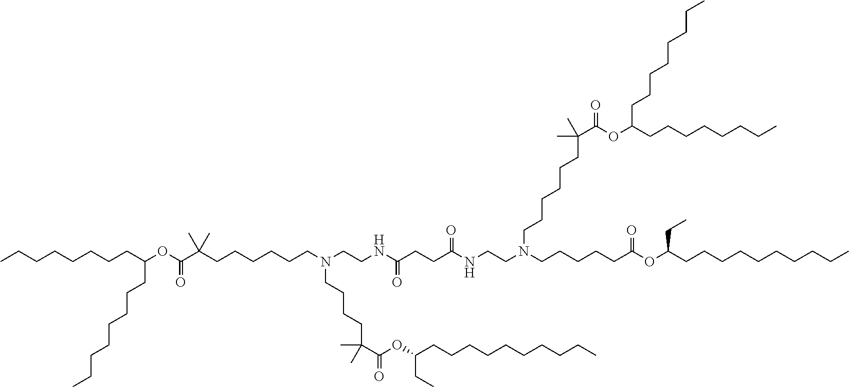 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | di(heptadecan-9-yl) 9- (5,5-dimethyl-6-oxo-6- (tridecan-3- yloxy)hexyl)-2,2,27,27- tetramethyl-13,16- dioxo-20-(6-oxo-6- (tridecan-3- yloxy)hexyl)- 9,12,17,20- tetraazaoctacosanedio- ate |
| A47 | 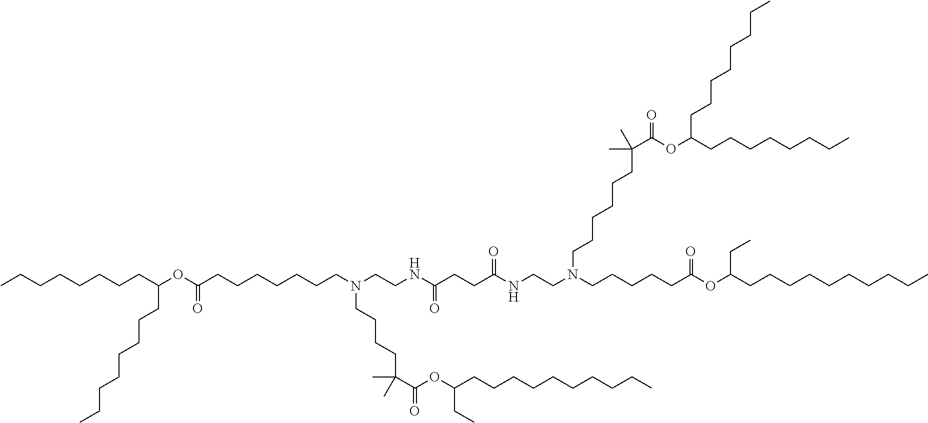 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] | di(heptadecan-9-yl) 20-(5,5-dimethyl-6- oxo-6-(tridecan-3- yloxy)hexyl)-2,2- dimethyl-13,16-dioxo- 9-(6-oxo-6-(tridecan-3- yloxy)hexyl)- 9,12,17,20- tetraazaoctacosanedio- ate |
| A48 | 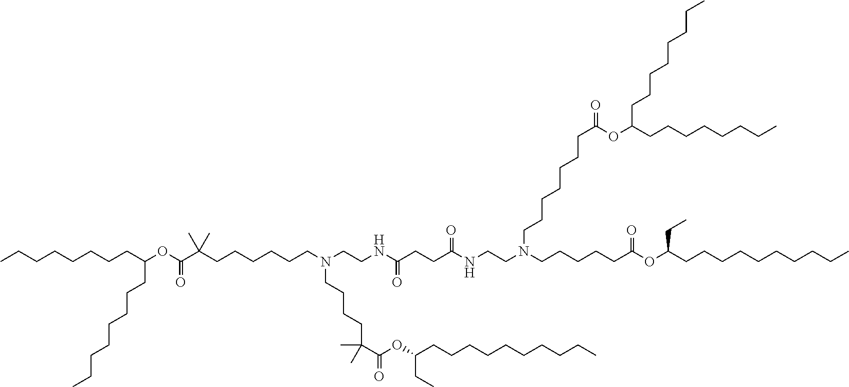 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | di(heptadecan-9-yl) 9- (5,5-dimethyl-6-oxo-6- (tridecan-3- yloxy)hexyl)-2,2- dimethyl-13,16-dioxo- 20-(6-oxo-6-(tridecan- 3-yloxy)hexyl)- 9,12,17,20- tetraazaoctacosanedio- ate |
| A49 | 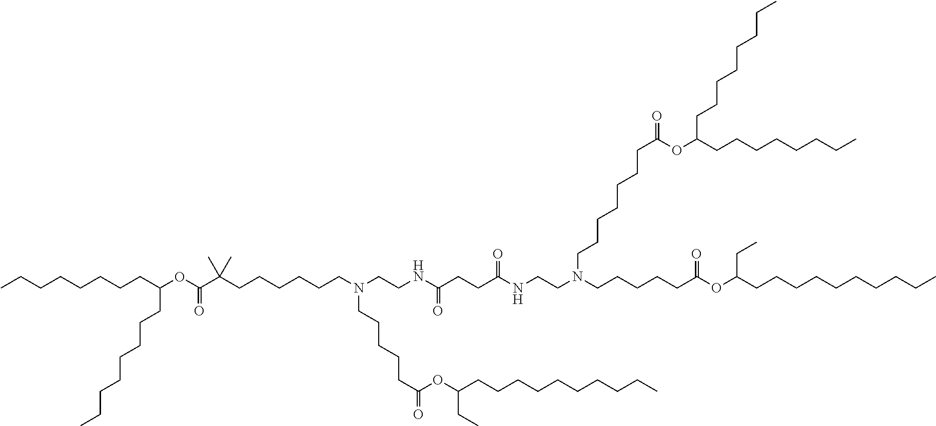 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OC(CC)CCCCCCCCCC] | di(heptadecna-9-yl) 2,2-dimethyl-13,16- dioxo-9,20-bis(6-oxo- 6-(tridecan-3- yloxy)hexyl)- 9,12,17,20- tetraazaoctacosanedio- ate |
| A50 | 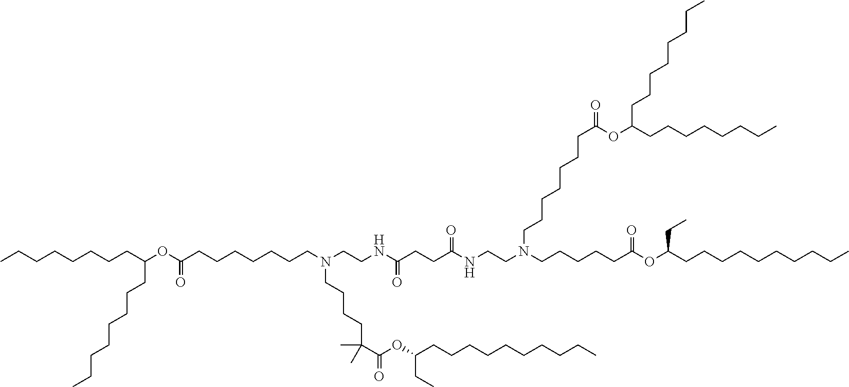 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | di(heptadecan-9-yl) 9- (5,5-dimethyl-6-oxo-6- (tridecan-3- yloxy)hexyl)-13,16- dioxo-20-(6-oxo-6- (tridecan-3- yloxy)hexyl)- 9,12,17,20- tetraazaoctacosanedio- ate |
| A51 | 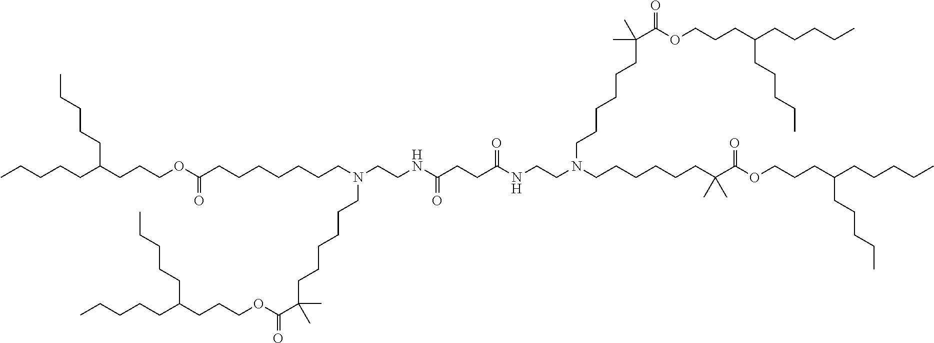 [SMILES: CCCCCC(CCCCC)CCCOC(=O)CCCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | bis(4-pentylnonyl) 9- (7,7-dimethyl-8-oxo-8- ((4- pentylnonyl)oxy)octyl)- 2,2,27,27-tetramethyl- 13,16-dioxo-20-(8- oxo-8-((4- pentylnonyl)oxy)octyl)- 9,12,17,20- tetraazaoctacosanedio- ate |
| A52 | 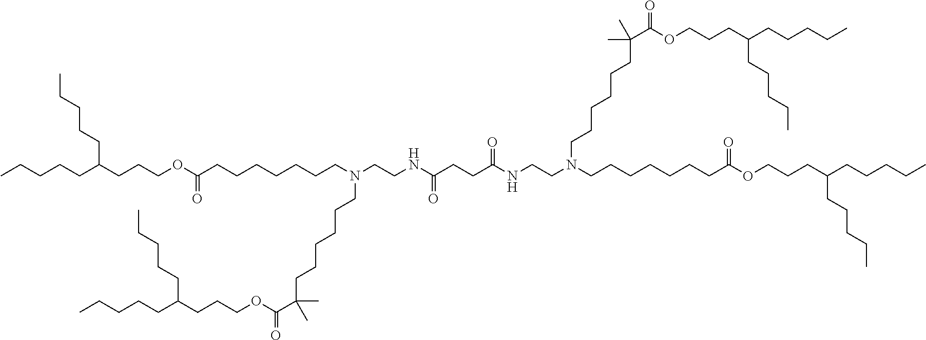 [SMILES: CCCCCC(CCCCC)CCCOC(=O)CCCCCCCN(CCCCCCC(C)(C)C(=O)OCCCC(CCCCC)CCCCC)CCNC(=O)CCC(=O)NCCN(CCCCCCCC(=O)OCCCC(CCCCC)CCCCC)CCCCCCC(C)(C)C(=O)OCCCC(CCCCC)CCCCC] | bis(4-pentylnonyl) 2,2,27,27-tetramethyl- 13,16-dioxo-9,20- bis(8-oxo-8-((4- pentylnonyl)oxy)octyl)- 9,12,17,20- tetraazaoctacosanedio- ate |
| A53 | 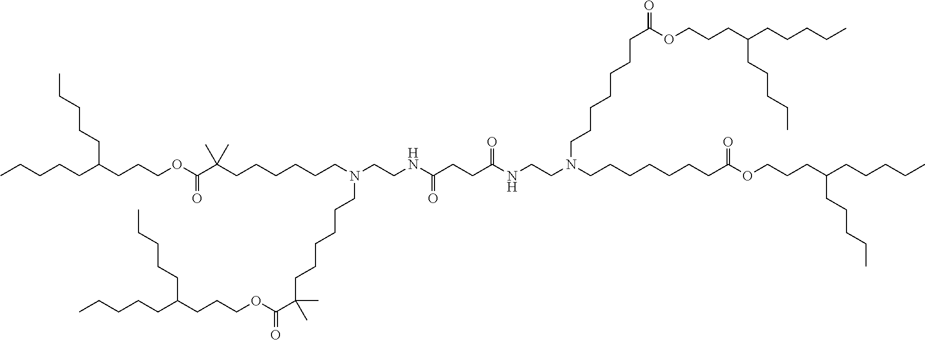 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | bis(4-pentylnonyl) 9- (7,7-dimethyl-8-oxo-8- ((4- pentylnonyl)oxy)octyl)- 2,2-dimethyl-13,16- dioxo-20-(8-oxo-8-((4- pentylnonyl)oxy)octyl)- 9,12,17,20- tetraazaoctacosanedio- ate |
| A54 | 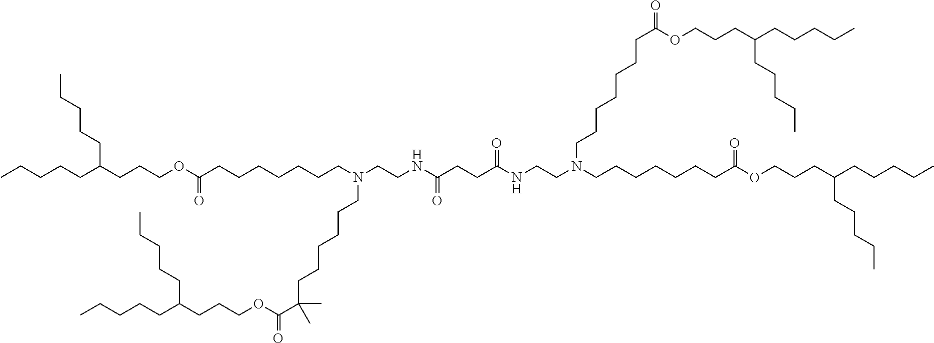 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCCC(=O)OCCCC(CCCCC)CCCCC)CCCCCCC(C)(C)C(=O)OCCCC(CCCCC)CCCCC] | bis(4-pentylnonyl) 2,2- dimethyl-13,16-dioxo- 9,20-bis(8-oxo-8-((4- pentylnonyl)oxy)octyl)- 9,12,17,20- tetraazaoctacosanedio- ate |
| A55 | 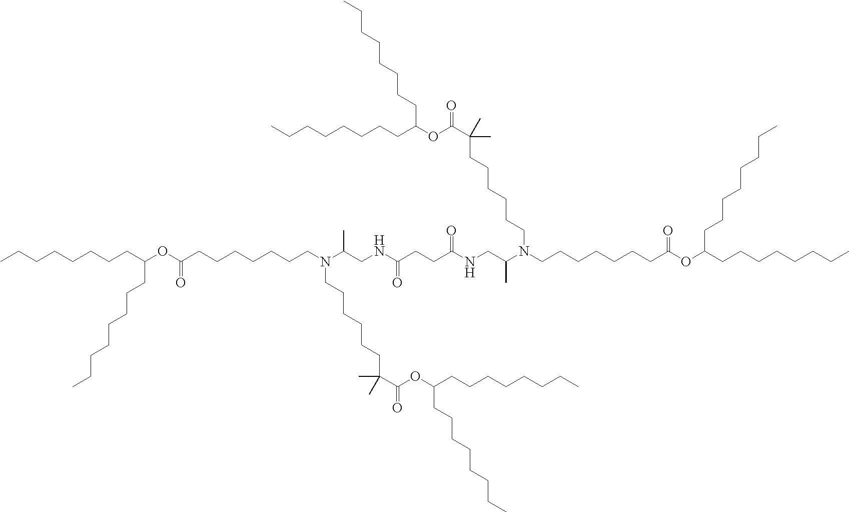 [SMILES: CCCCCCCCC(CCCCCCCC)OC(=O)CCCCCCCN(CCCCCCC(C)(C)C(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- oxooctyl)-20-(8- (heptadecan-9-yloxy)- 8-oxooctyl)- 2,2,10,19,27,27- hexamethyl-13,16- dioxo-9,12,17,20- tetraazaoctacosanedio- ate |
| A56 | 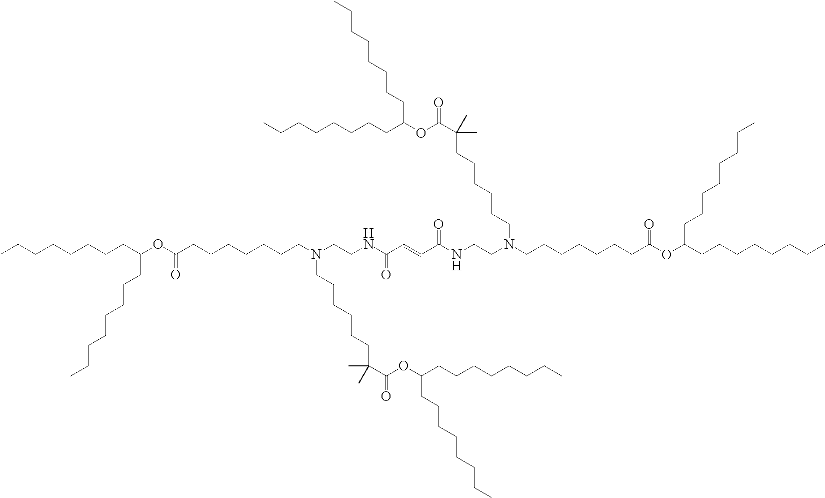 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,20-bis(8- (heptadecan-9-yloxy)- 8-oxooctyl)- 2,2,10,19,27,27- hexamethyl-13,16- dioxo-9,12,17,20- htetraazaoctacosanedio- ate |
| A57 | 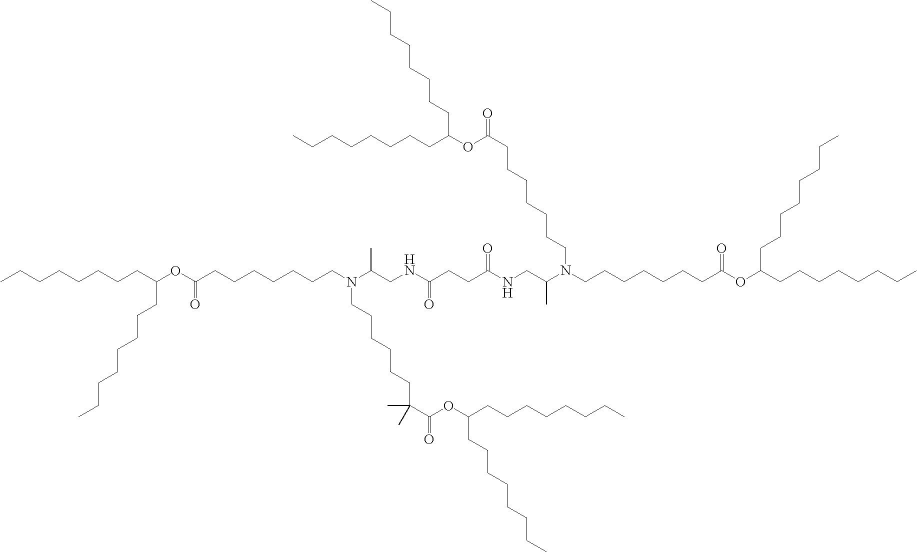 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- (heptadecan-9-yloxy)- 8-oxooctyl)-2,2,10,19- tetramethyl-13,16- dioxo-9,12,17,20- tetraazaoctacosanedio- ate |
| A58 | 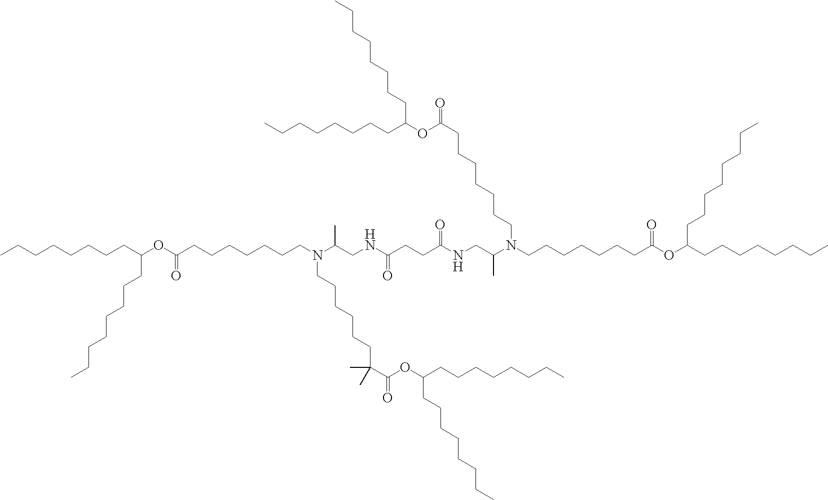 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,20-bis(8- (heptadecan-9-yloxy)- 8-oxooctyl)-2,2,10,19- tetramethyl-13,16- dioxo-9,12,17,20- tetraazaoctacosanedio- ate |
| A59 | 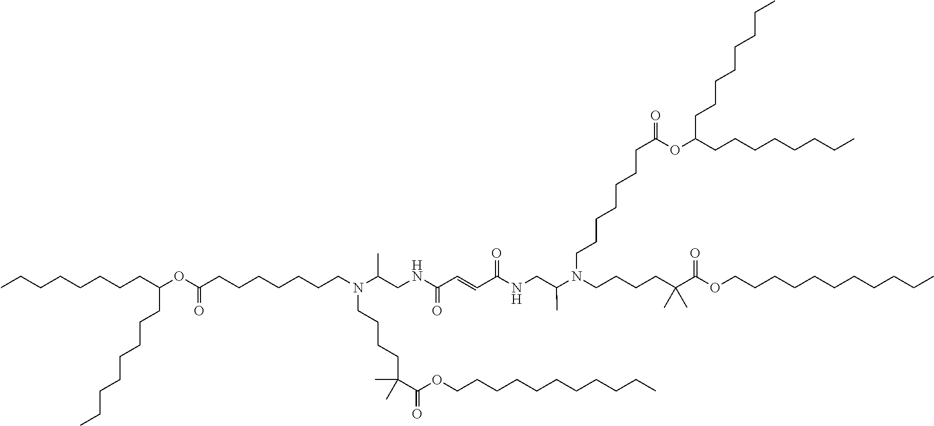 [SMILES: CCCCCCCCCCCOC(=O)C(C)(C)CCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | di(heptadecan-9-yl) (E)-9,20-bis(5,5- dimethyl-6-oxo-6- (undecyloxy)hexyl)- 10,19-dimethyl-13,16- dioxo-9,12,17,20- tetraazaoctacos-14- enedioate |
| A60 | 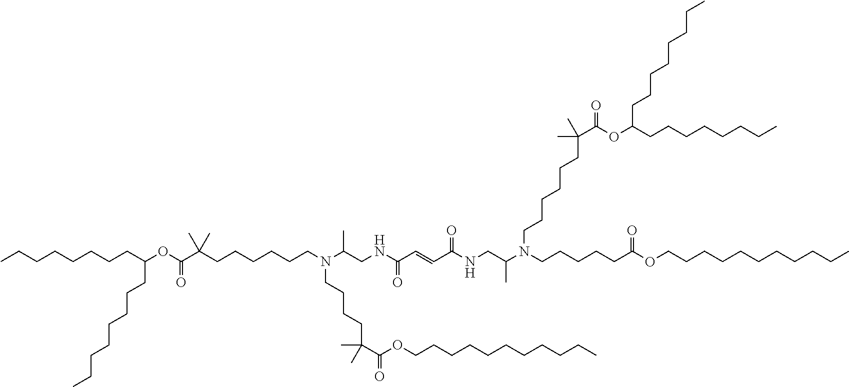 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCC(C)(C)C(=O)OCCCCCCCCCCC] | di(heptadecan-9-yl) (E)-9-(5,5-dimethyl-6- oxo-6- (undecyloxy)hexyl)- 2,2,10,19,27,27- hexamethyl-13,16- dioxo-20-(6-oxo-6- (undecyloxy)hexyl)- 9,12,17,20- tetraazaoctacos-14- enedioate |
| A61 | 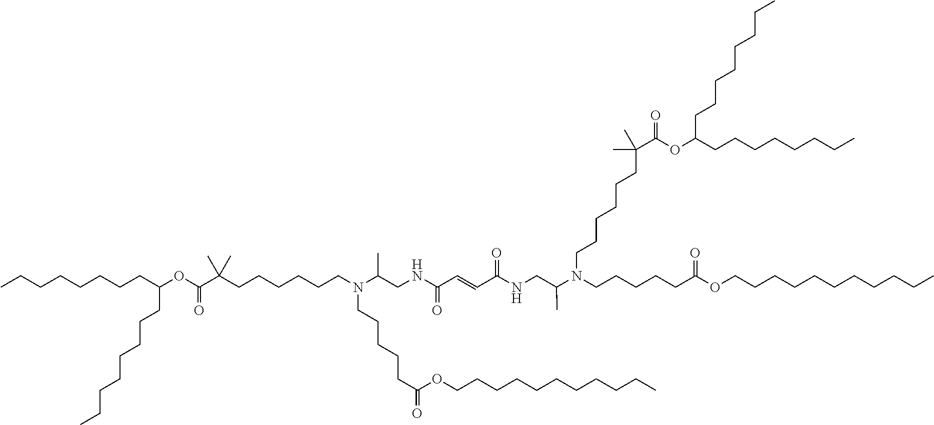 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCCC(=O)OCCCCCCCCCCC] | di(heptadecan-9-yl) (E)-2,2,10,19,27,27- hexamethyl-13,16- dioxo-9,20-bis(6-oxo- 6-(undecyloxy)hexyl)- 9,12,1,7,20- tetraazaoctacos-14- enedioate |
| A62 | 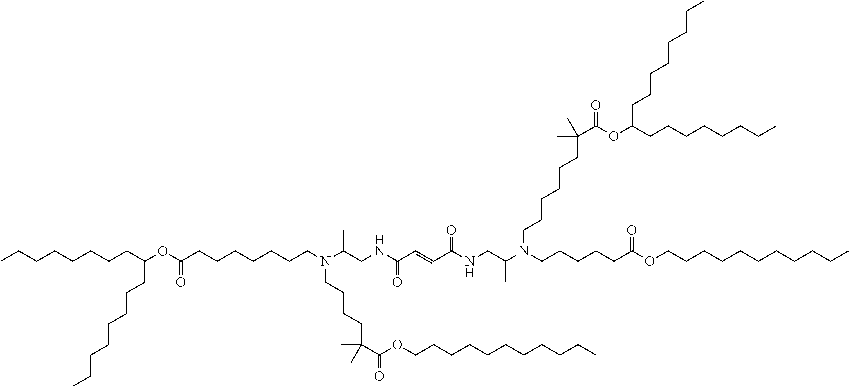 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | di(heptadecan-9-yl) (E)-20-(5,5-dimethyl-6- oxo-6- (undecyloxy)hexyl)- 2,2,10,19-tetramethyl- 13,16-dioxo-9-(6-oxo- 6-(undecyloxy)hexyl)- 9,12,17,20- tetraazaoctacos-14- enedioate |
| A63 | 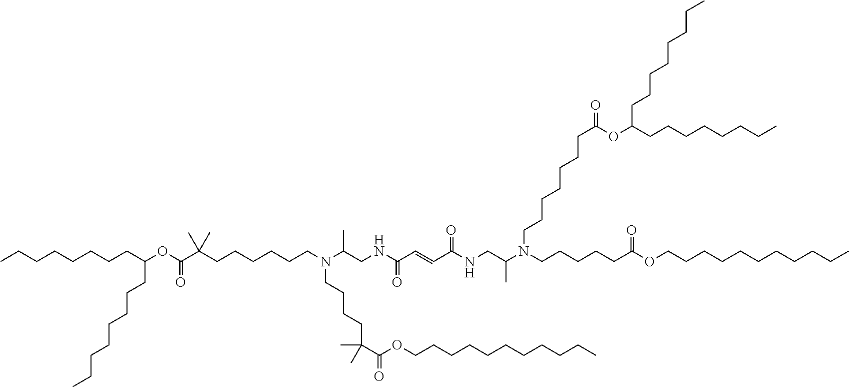 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCC(C)(C)C(=O)OCCCCCCCCCCC] | di(heptadecan-9-yl) (E)-9-(5,5-dimethyl-6- oxo-6- (undecyloxy)hexyl)- 2,2,10,19-tetramethyl- 13,16-dioxo-20-(6- oxo-6- (undecyloxy)hexyl)- 9,12,17,20- tetraazaoctacos-14- enedioate |
| A64 | 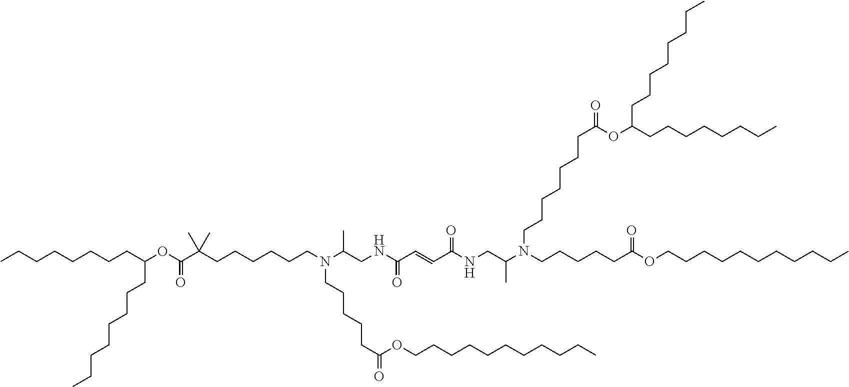 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCCC(=O)OCCCCCCCCCCC] | di(heptadecan-9-yl) (E)-2,2,10,19- tetramethyl-13,16- dioxo-9,20-bis(6-oxo- 6-(undecyloxy)hexyl)- 9,12,17,20- tetraazaoctacos-14- enedioate |
| A65 | 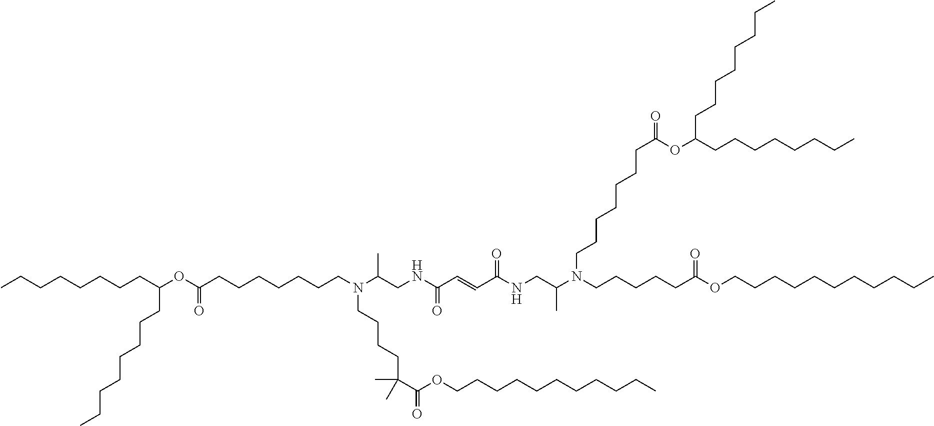 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | di(heptadecan-9-yl) (E)-9-(5,5-dimethyl-6- oxo-6- (undecyloxy)hexyl)- 10,19-dimethyl-13,16- dioxo-20-(6-oxo-6- (undecyloxy)hexyl)- 9,12,17,20- tetraazaoctacos-14- enedioate |
| A66 | 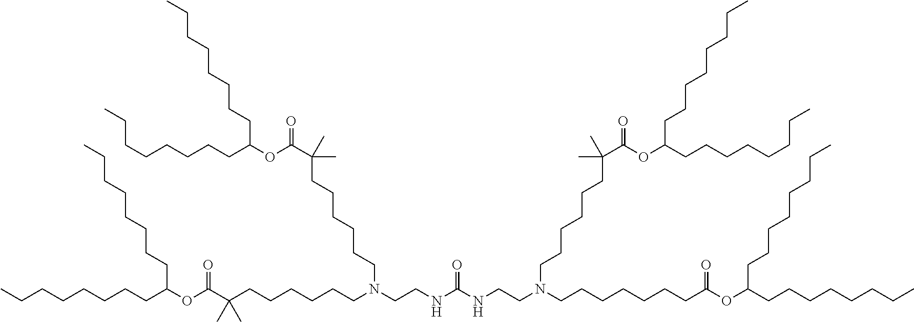 [SMILES: CCCCCCCCC(CCCCCCCC)OC(=O)CCCCCCCN(CCCCCCC(C)(C)C(=O)OC(CCCCCCCC)CCCCCCCC)CCNC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- oxooctyl)-17-(8- (heptadecan-9-yloxy)- 8-oxooctyl)-2,2,24,24- tetramethyl-13-oxo- 9,12,14,17- tetraazapentacosanedi- oate |
| A67 | 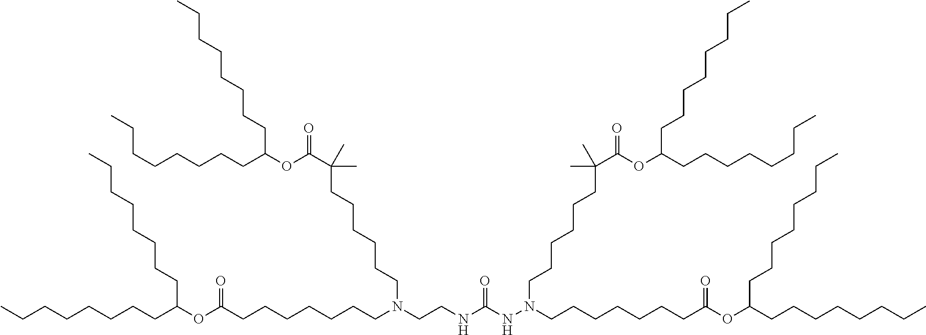 [SMILES: CCCCCCCCC(CCCCCCCC)OC(=O)CCCCCCCN(CCCCCCC(C)(C)C(=O)OC(CCCCCCCC)CCCCCCCC)CCNC(=O)NN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,17-bis(8- (heptadecan-9-yloxy)- 8-oxooctyl)-2,2,24,24- tetramethyl-13-oxo- 9,12,14,17- tetraazapentacosanedi- oate |
| A68 | 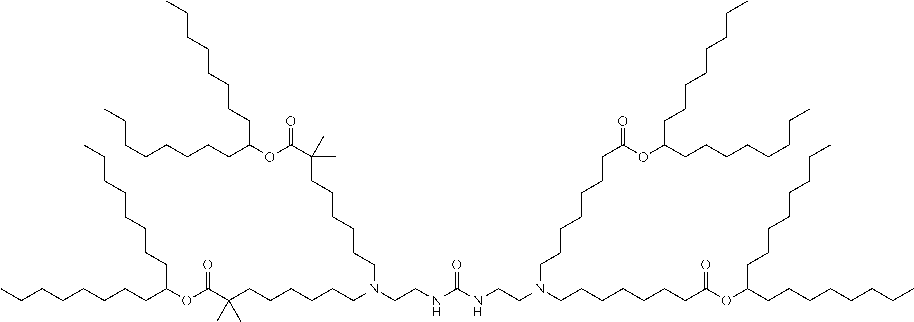 [SMILES: CCCCCCCCC(CCCCCCCC)OC(=O)CCCCCCCN(CCCCCCCC(=O)OC(CCCCCCCC)CCCCCCCC)CCNC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- oxooctyl)-17-(8- (heptadecan-9-yloxy)- 8-oxooctyl)-2,2- dimethyl-13-oxo- 9,12,14,17- tetraazapentacosanedi- oate |
| A69 | 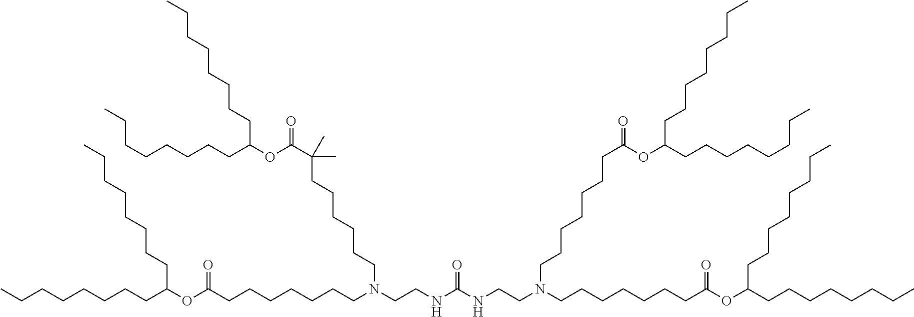 [SMILES: CCCCCCCCC(CCCCCCCC)OC(=O)CCCCCCCN(CCCCCCCC(=O)OC(CCCCCCCC)CCCCCCCC)CCNC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,17-bis(8- (heptadecan-9-yloxy)- 8-oxooctyl)-2,2- dimethyl-13-oxo- 9,12,14,17- tetraazapentacosanedi- oate |
| A70 | 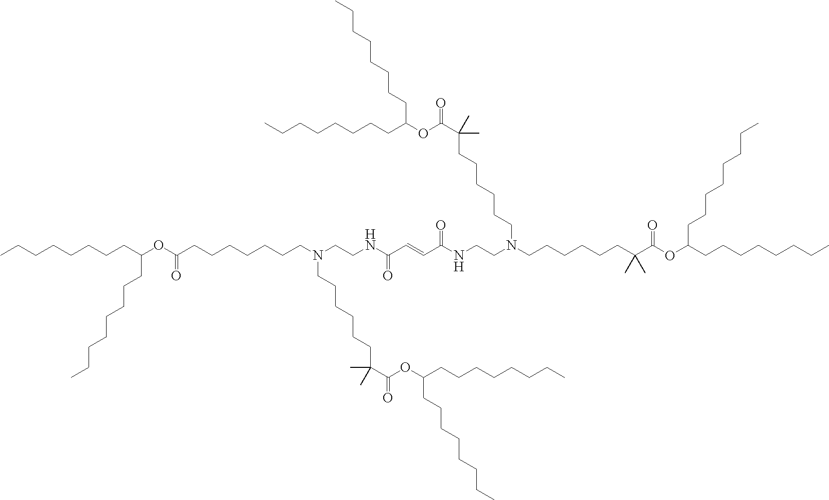 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) (E)-9-(8-(heptadecan- 9-yloxy)-7,7-dimethyl- 8-oxooctyl)-20-(8- (heptadecan-9-yloxy)- 8-oxooctyl)-2,2,27,27- tetramethyl-13,16- dioxo-9,12,17,20- tetraazaoctacos-14- enedioate |
| A71 | 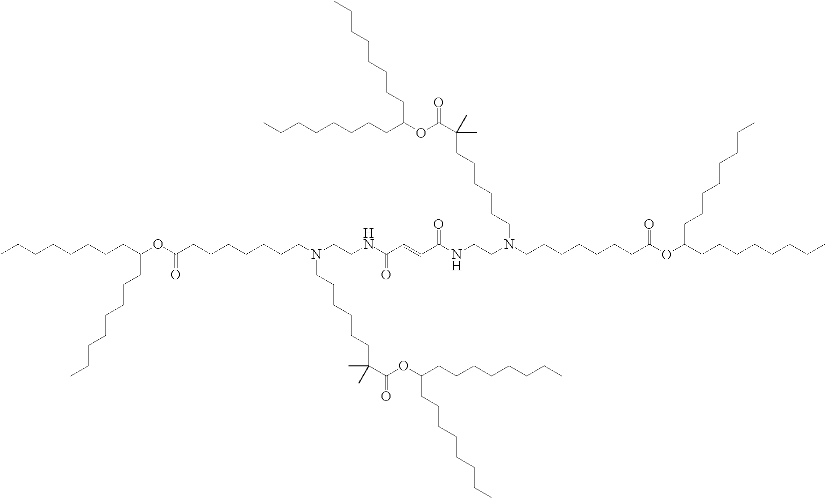 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) (E)-9,20-bis(8- (heptadecan-9-yloxy)- 8-oxooctyl)-2,2,27,27- tetramethyl-13,16- dioxo-9,12,17,20- tetraazaoctacos-14- enedioate |
| A72 | 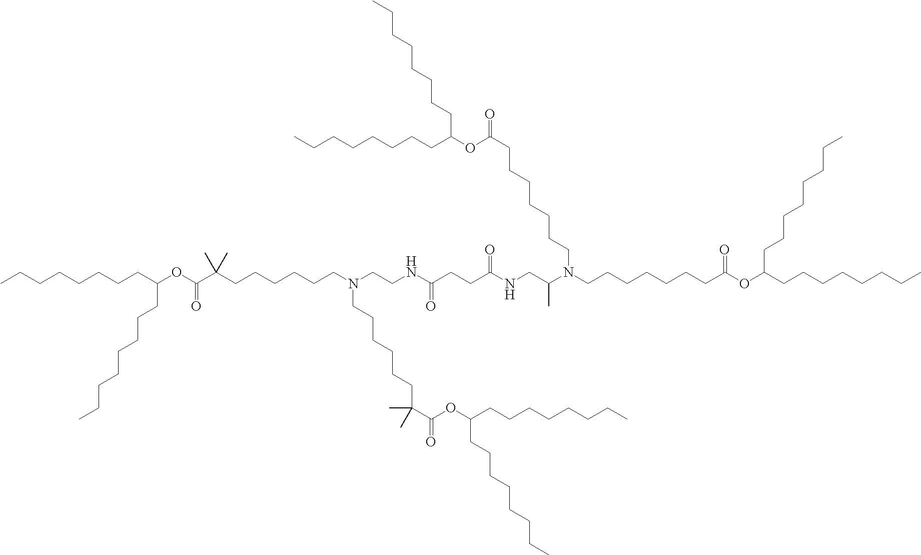 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) (E)-9-(8-(heptadecan- 9-yloxy)-7,7-dimethyl- 8-oxooctyl)-20-(8- (heptadecan-9-yloxy)- 8-oxooctyl)-2,2- dimethyl-13,16-dioxo- 9,12,17,20- tetraazaoctacos-14- enedioate |
| A73 | 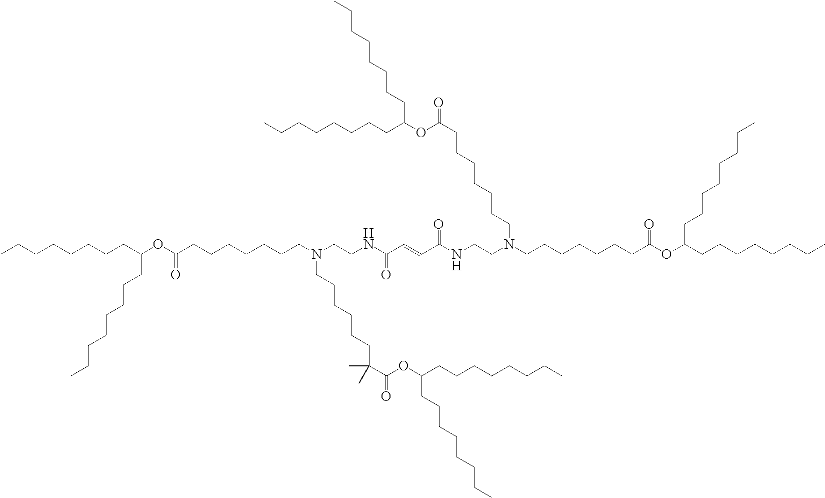 [SMILES: CCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) (E)-9,20-bis(8- (heptadecan-9-yloxy)- 8-oxooctyl)-2,2- dimethyl-13,16-dioxo- 9,12,17,20- tetraazaoctacos-14- enedioate |
| A74 | 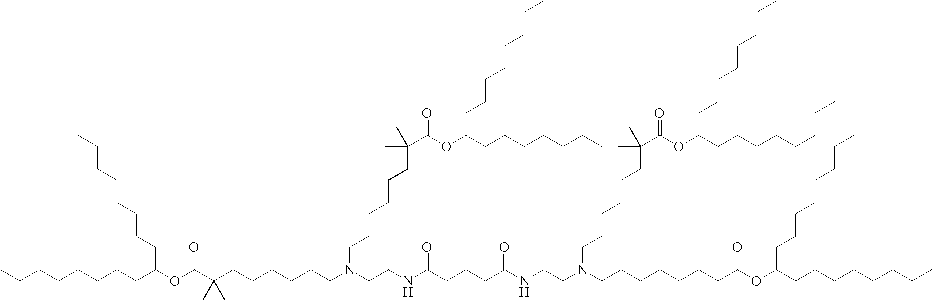 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- oxooctyl)-21-(8- (heptadecan-9-yloxy)- 8-oxooctyl)-2,2,28,28- tetramethyl-13,17- dioxo-9,12,18,21- tetraazanonacosanedi- oate |
| A75 | 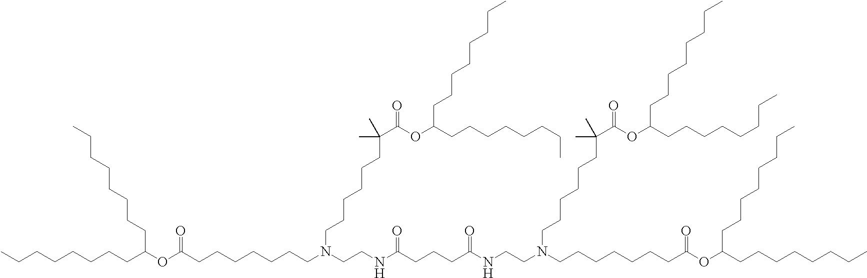 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCCC(=O)NCCN(CCCCCCCC(=O)OC(CCCCCCCC)CCCCCCCC)CCCCCCC(C)(C)C(=O)OC(CCCCCCCC)CCCCCCCC] | di(hpetadecan-9-yl) 9,21-bis(8- (heptadecan-9-yloxy)- 8-oxooctyl)-2,2,28,28- tetramethyl-13,17- dioxo-9,12,18,21- tetraazanonacosanedi- oate |
| A76 | 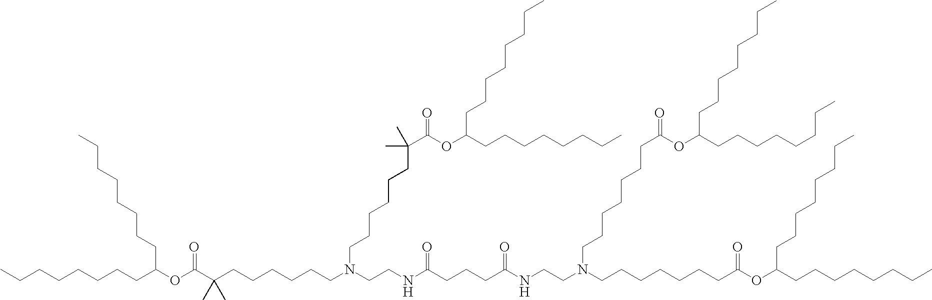 [SMILES: CCCCCCCCC(CCCCCCCC)OC(=O)CCCCCCCN(CCCCCCCC(=O)OC(CCCCCCCC)CCCCCCCC)CCNC(=O)CCCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecna-9- yloxy)-7,7-dimethyl-8- oxooctyl)-21-(8- (heptadecan-9-yloxy)- 8-oxooctyl)-2,2- dimethyl-13,17-dioxo- 9,12,18,21- tetraazanonacosanedi- oate |
| A77 | 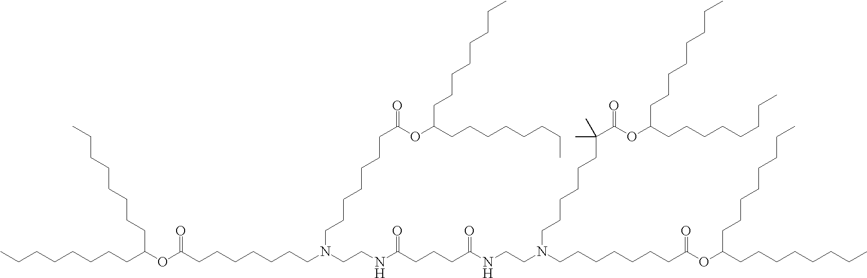 [SMILES: CCCCCCCCC(CCCCCCCC)OC(=O)CCCCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,21-bis(8- (heptadecan-9-yloxy)- 8-oxooctyl)-2,2- dimethyl-13,17-dioxo- 9,12,18,21- tetraazanonacosanedi- oate |
| A78 | 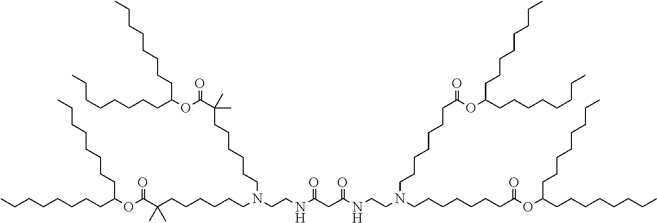 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- oxooctyl)-19-(8- (heptadecan-9-yloxy)- 8-oxooctyl)-2,2,26,26- tetramnethyl-13,15- dioxo-9,12,16,19- tetraazaheptacosanedi- oate |
| A79 | 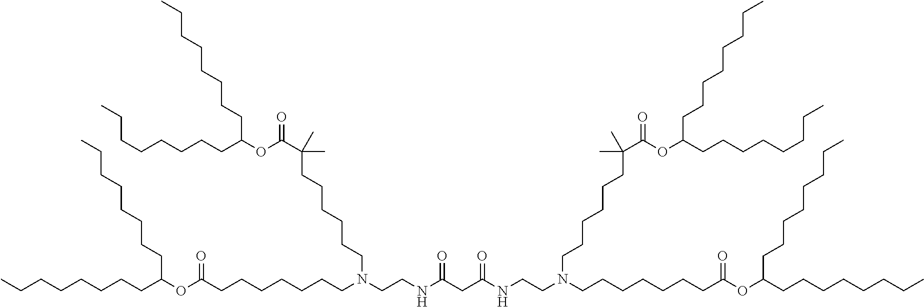 [SMILES: CCCCCCCCC(CCCCCCCC)OC(=O)CCCCCCCN(CCCCCCC(C)(C)C(=O)OC(CCCCCCCC)CCCCCCCC)CCNC(=O)CC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,19-bis(8- (heptadecan-9-yloxy)- 8-oxooctyl)-2,2,26,26- tetramethyl-13,15- dioxo-9,12,16,19- tetraazaheptacosanedi- oate |
| A80 | 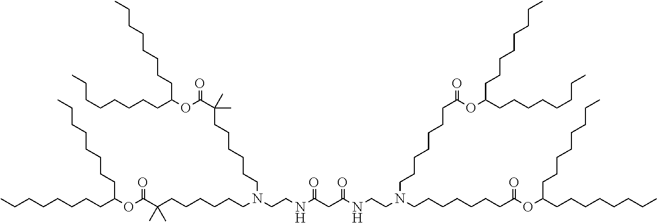 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(hpetadecan-9-yl) 9- (8-(heptadecan-9- yloxy)-7,7-dimethyl-8- oxooctyl)-19-(8- (heptadecan-9-yloxy)- 8-oxooctyl)-2,2- dimethyl-13,15-dioxo- 9,12,16,19- tetraazaheptacosanedi- oate |
| A81 | 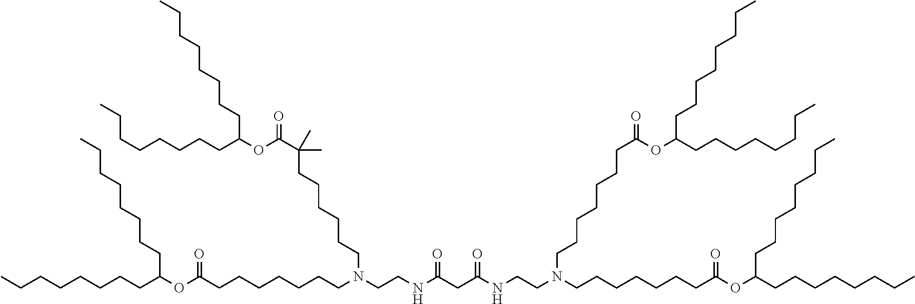 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,19-bis(8- (heptadecan-9-yloxy)- 8-oxooctyl)-2,2- dimethyl-13,15-dioxo- 9,12,16,19- tetraazaheptacosanedi- oate |
| A82 | 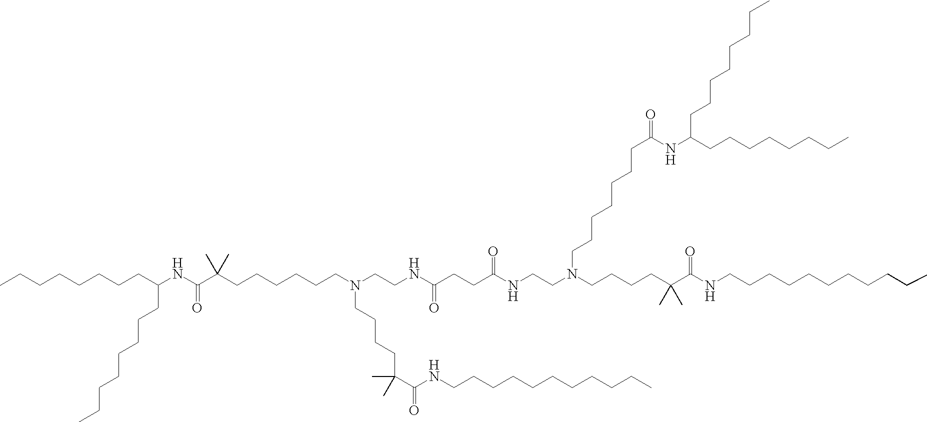 [SMILES: CCCCCCCCCCCNC(=O)C(C)(C)CCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] | N¹-(2-((5,5-dimethyl-6- oxo-6- (undecylamino)hexyl)(8- (heptadecan-9- ylamino)-7,7-dimethyl- 8- oxooctyl)amino)ethyl)- N⁴-(2-((5,5-dimethyl-6- oxo-6- (undecylamino)hexyl)(8- (heptadecan-9- ylamino)-8- oxooctyl)amino)ethyl)suc- cinamide |
| A83 | 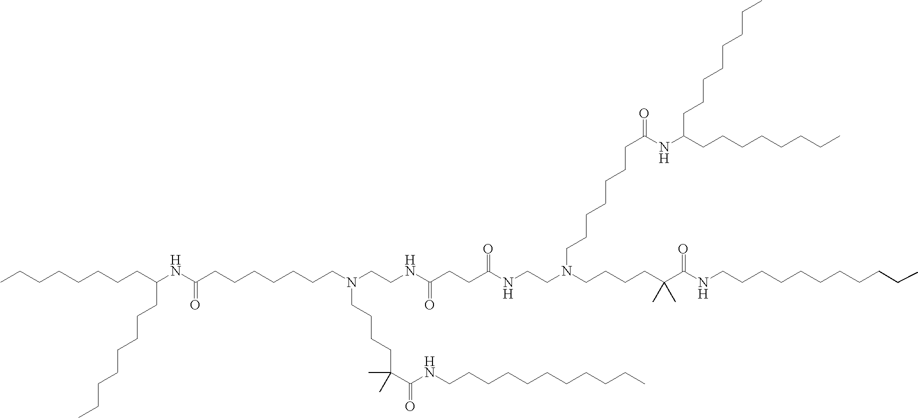 [SMILES: CCCCCCCCCCCNC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] | N¹,N⁴-bis(2-((5,5- dimethyl-6-oxo-6- (undecylamino)hexyl)(8- (heptadecan-9- ylamino)-8- oxooctyl)amino)ethyl)suc- cinamide |
| A84 | 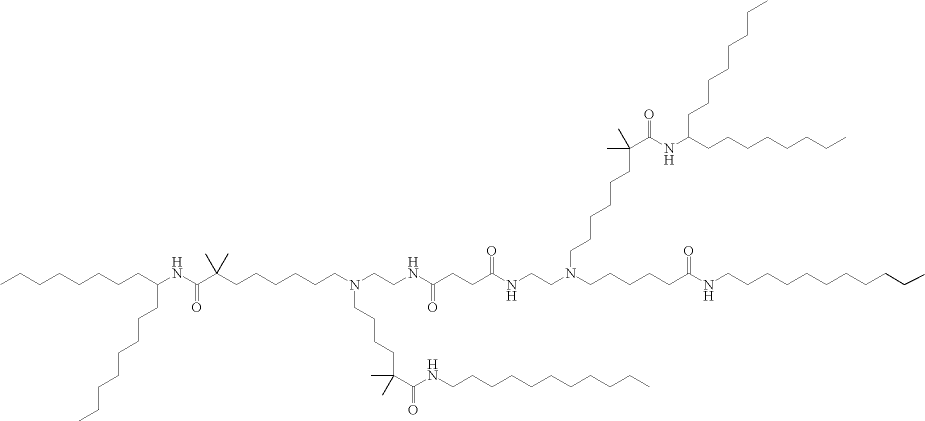 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] | N¹-(2-((5,5-dimethyl-6- oxo-6- (undecylamino)hexyl)(8- (heptadecan-9- ylamino)-7,7-dimethyl- 8- oxooctyl)amino)ethyl)- N⁴-(2-((8-(heptadecan- 9-ylamino)-7,7- dimethyl-8-oxooctyl)(6- oxo-6- (undecylamino)hexyl)a- mino)ethyl)succinamide |
| A85 | 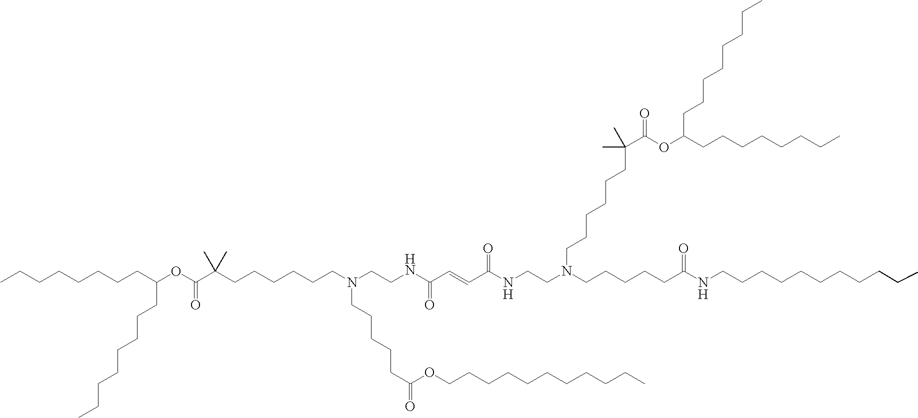 [SMILES: CCCCCCCCCCCNC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] | N¹,N⁴-bis(2-((8- (heptadecan-9- ylamino)-7,7-dimethyl- 8-oxooctyl)(6-oxo-6- (undecylamino)hexyl)a- mino)ethyl)succinamide |
| A86 | 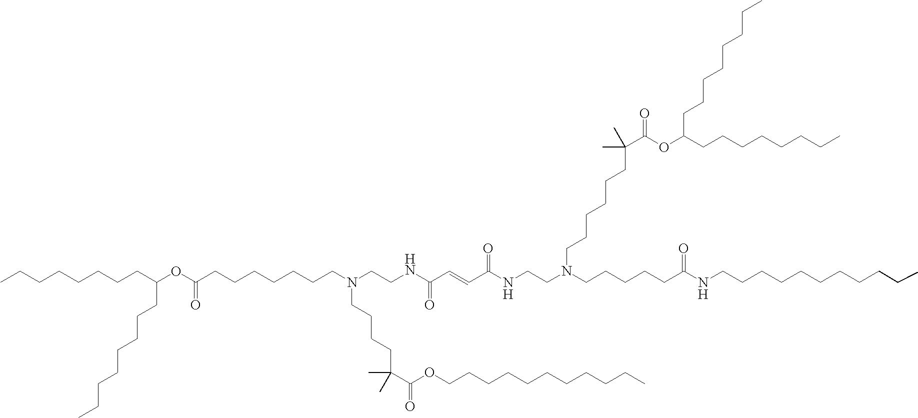 [SMILES: CCCCCCCCCCCNC(=O)CCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | N¹-(2-((5,5-dimethyl-6- oxo-6- (undecylamino)hexyl)(8- (heptadecan-9- ylamino)-8- oxooctyl)amino)ethyl)- N⁴-(2-((8-(heptadecan- 9-ylamino)-7,7- dimethyl-8-oxooctyl)(6- oxo-6- (undecylamino)hexyl)a- mino)ethyl)succinamide |
| A87 | 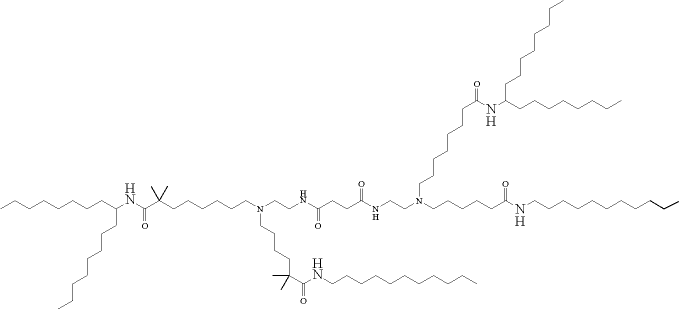 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] | N¹-(2-((5,5-dimethyl-6- oxo-6- (undecylamino)hexyl)(8- (heptadecan-9- ylamino)-7,7-dimethyl- 8- oxooctyl)amino)ethyl)- N⁴-(2-((8-(heptadecan- 9-ylamino)-8- oxooctyl)(6-oxo-6- (undecylamino)hexyl)a- mino)ethyl)succinamide |
| A88 | 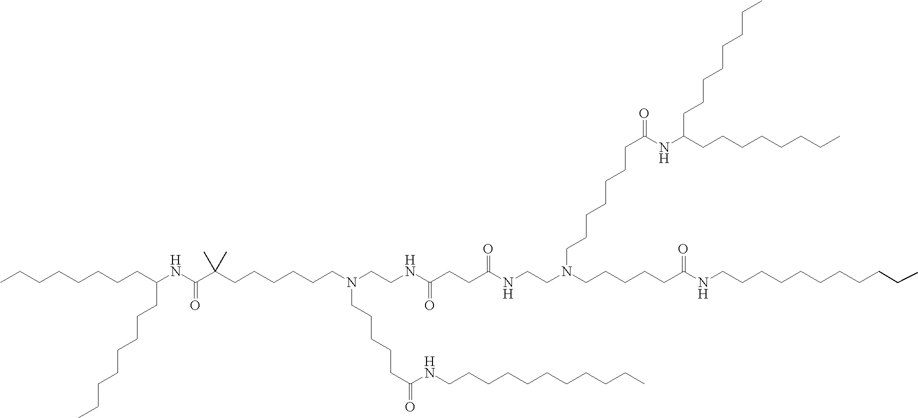 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)NCCCCCCCCCCC] | N¹-(2-((8-(heptadecan- 9-ylamino)-7,7- dimethyl-8-oxooctyl)(6- oxo-6- (undecaylamino)hexyl)a- mino)ethyl)-N⁴-(2-((8- (heptadecan-9- ylamino)-8- oxooctyl)(6-oxo-6- (undecylamino)hexyl)a- mino)ethyl)succinamide |
| A89 | 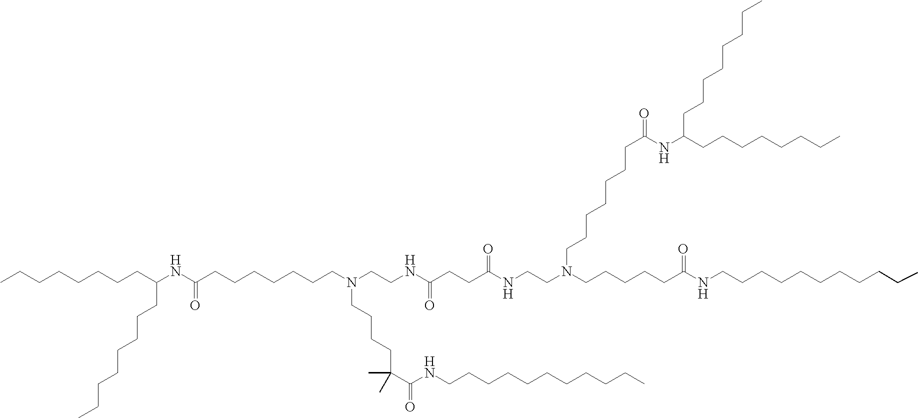 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] | N¹-(2-((5,5-dimethyl-6- oxo-6- (undecylamino)hexyl)(8- (heptadecan-9- ylamino)-8- oxooctyl)amino)ethyl)- N⁴-(2-((8-(heptadecan- 9-ylamino)-8- oxooctyl)(6-oxo-6- (undecylamino)hexyl)a- mino)ethyl)succinamide |
| A90 | 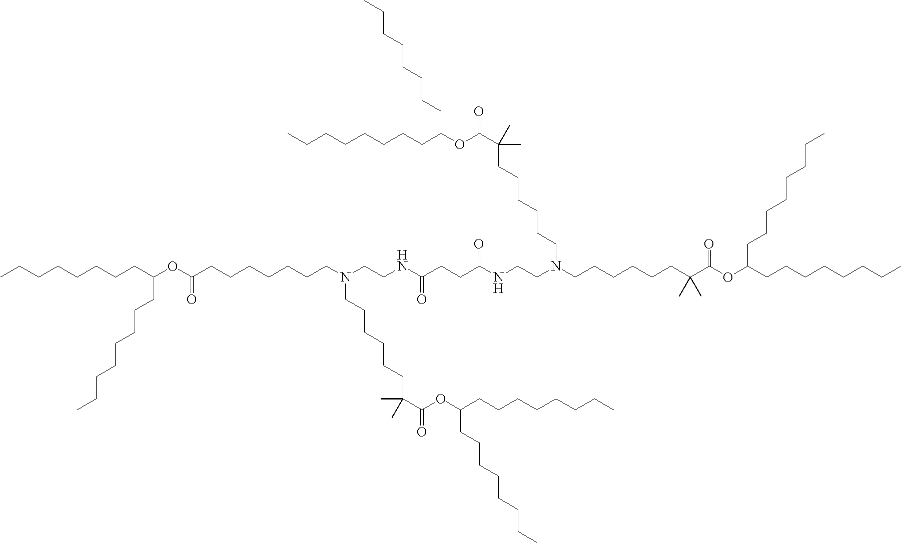 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- ylxoy)-7,7-dimethyl-8- oxooctyl)-20-(8- (heptadecan-9-yloxy)- 8-oxooctyl)-2,2,27,27- tetramethyl-13,16- dioxo-9,12,17,20- tetraazaoctacosanedio- ate |
| A91 | 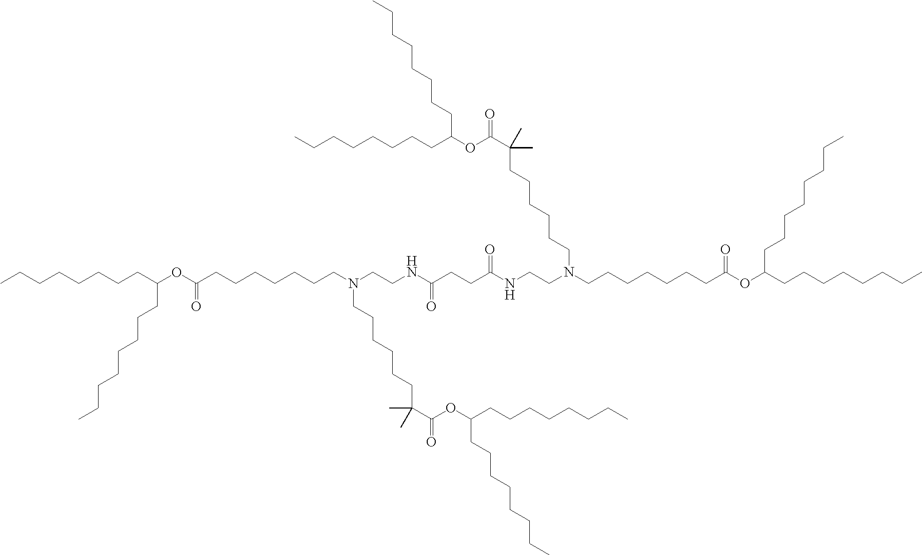 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,20-bis(8- (heptadecan-9-yloxy)- 8-oxooctyl)-2,2,27,27- tetramethyl-13,16- dioxo-9,12,17,20- tetraazaoctacosanedio- ate |
| A92 | 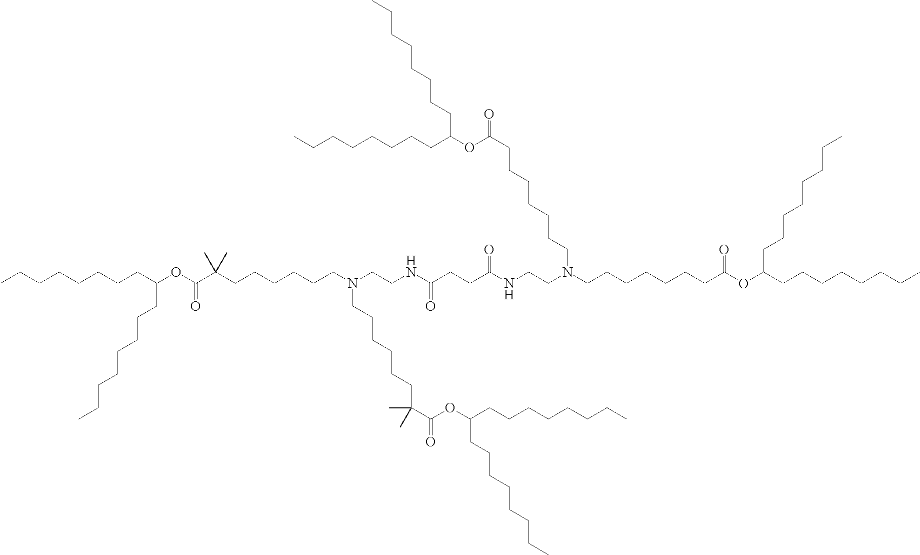 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9- (8-(heptadecan-9- yloxy)-7,7-dimethyl-8- oxooctyl)-20-(8- (heptadecan-9-yloxy)- 8-oxootyl)-2,2- dimethyl-13,16-dioxo- 9,12,17,20- tetraazaoctacosanedio- ate |
| A93 | 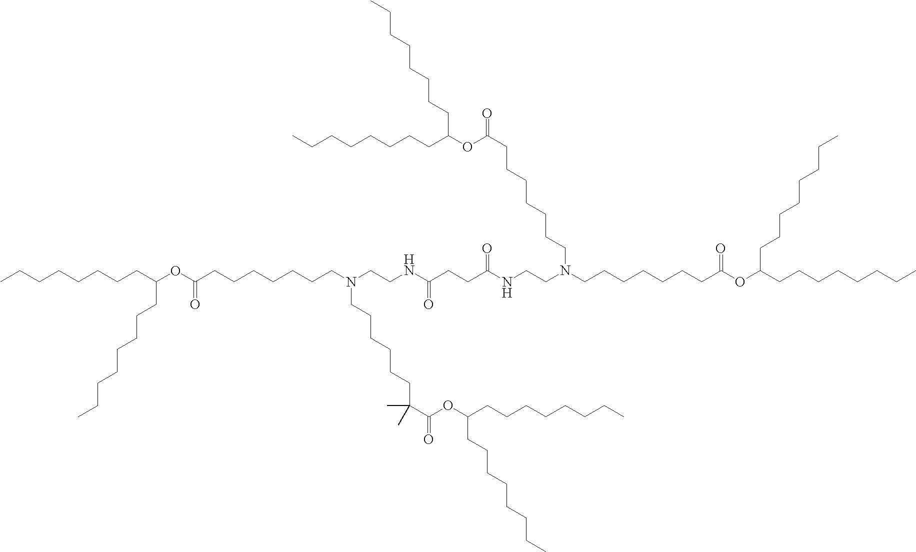 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,20-bis(8- (heptadecan-9-yloxy)- 8-oxooctyl)-2,2- dimethyl-13,16-dioxo- 9,12,17,20- tetraazaoctacosanedio- ate |
| A94 | 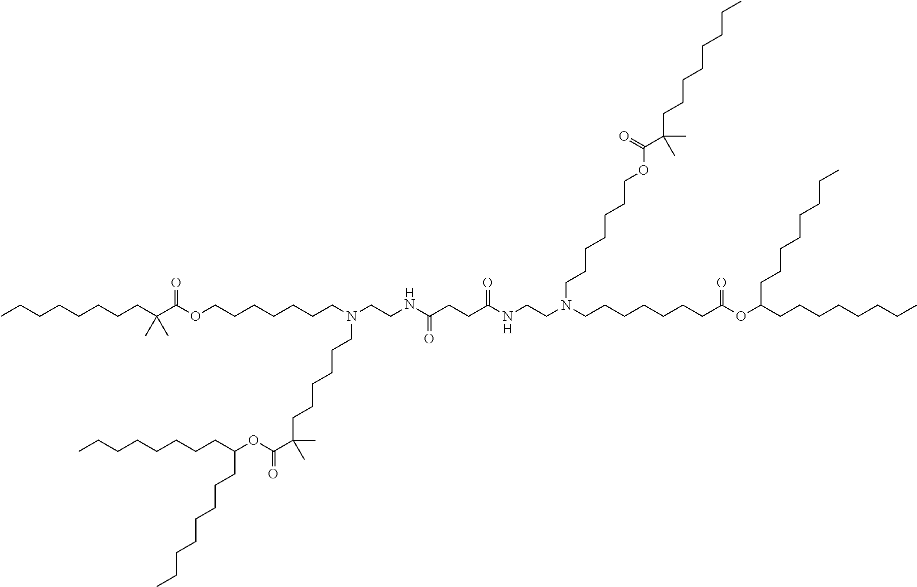 [SMILES: CCCCCCCCC(CCCCCCCC)OC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | di(heptadecan-9-yl) 9,20-bis(7-((2,2- dimethyldecanoyl)oxy) heptyl)-2,2-dimethyl- 13,16-dioxo- 9,12,17,20- tetraazaoctacosanedio- ate |
| A95 | 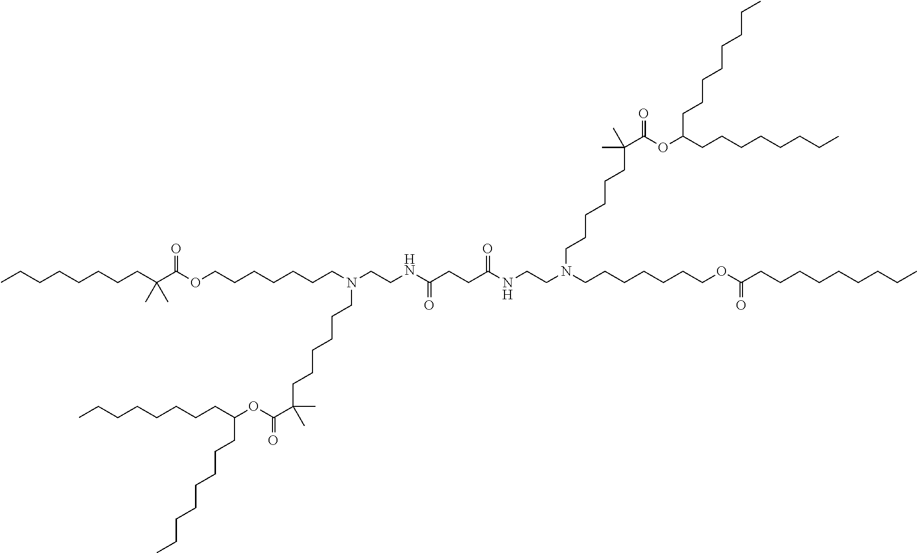 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | di(heptadecan-9-yl) 9- (7- (decanoyloxy)heptyl)- 20-(7-((2,2- dimethyldecanoyl)oxy) heptyl)-2,2,27,27- tetramethyl-13,16- dioxo-9,12,17,20- tetraazaoctacosanedio- ate |
| A96 | 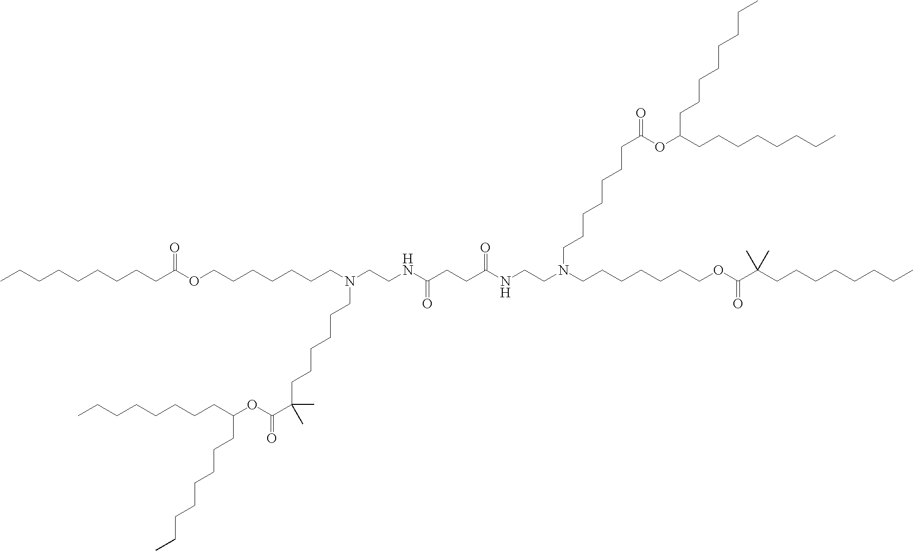 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] | di(heptadecan-9-yl) 9- (7- (decanoyloxy)heptyl)- 20-(7-((2,2- dimethyldecanoyl)oxy) heptyl)-2,2-dimethyl- 13,16-dioxo- 9,12,17,20- tetraazaoctaccosanedio- ate |
| A97 | 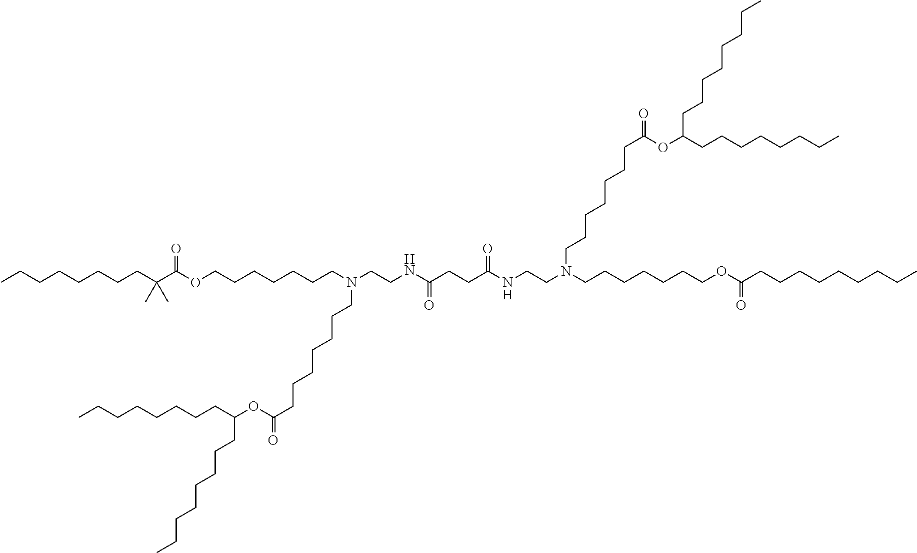 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] | di(heptadecan-9-yl) 9- (7- (decanoyloxy)heptyl)- 20-(7-((2,2- dimethyldecanoyl)oxy) heptyl)-13,16-dioxo- 9,12,17,20- tetraazaoctasanedio- ate |
| A98 | 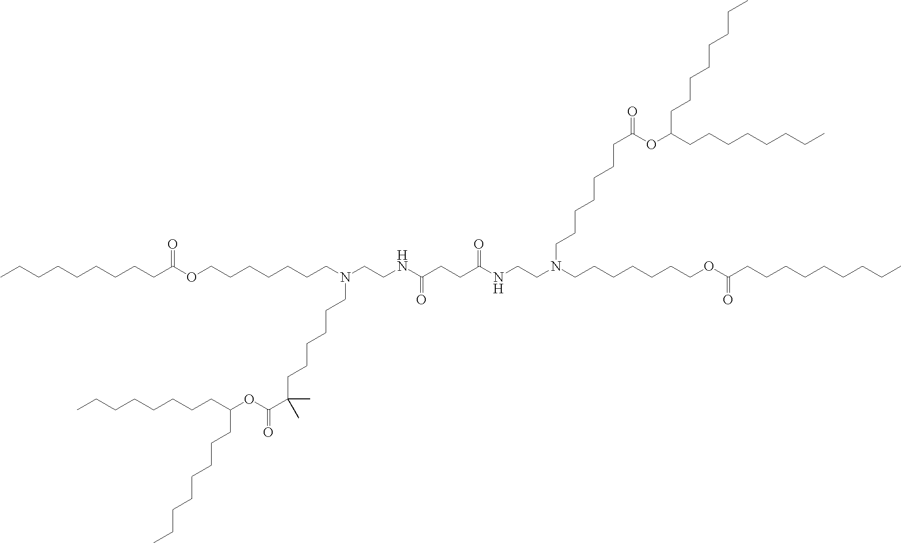 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)CCCCCCCCC)CCCCCCC(C)(C)C(=O)OC(CCCCCCCC)CCCCCCCC] | di(heptadecan-9-yl) 9,20-bis(7- (decanoyloxy)heptyl)- 2,2-dimethyl-13,16- dioxo-9,12,17,20- tetraazaoctacosanedio- ate |
| A99 | 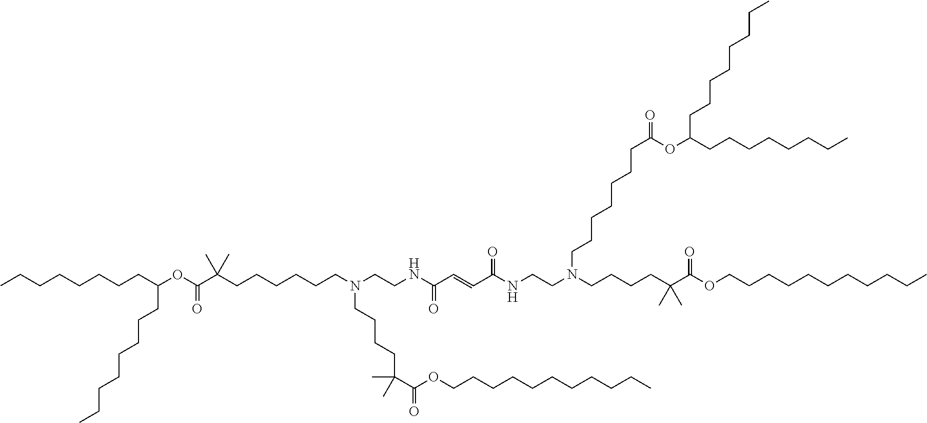 [SMILES: CCCCCCCCCCCOC(=O)C(C)(C)CCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) (E)-9,20-bis(5,5- dimethyl-6-oxo-6- (undecyloxy)hexyl)- 2,2-dimethyl-13,16- dioxo-9,12,17,20- tetraazaoctacos-14- enedioate |
| A100 | 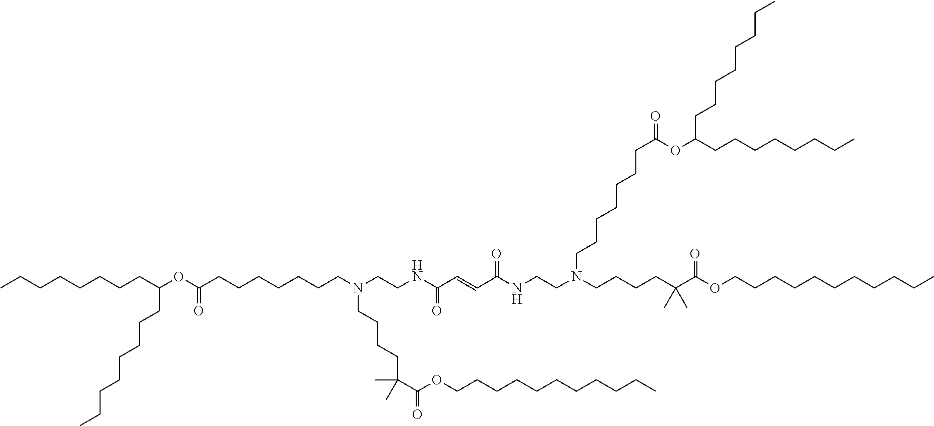 [SMILES: CCCCCCCCCCCOC(=O)C(C)(C)CCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | di(heptadecan-9-yl) (E)-9,20-bis(5,5- dimethyl-6-oxo-6- (undecyloxy)hexyl)- 13,16-dioxo- 9,12,17,20- tetraazaoctacos-14- enedioate |
| A101 | 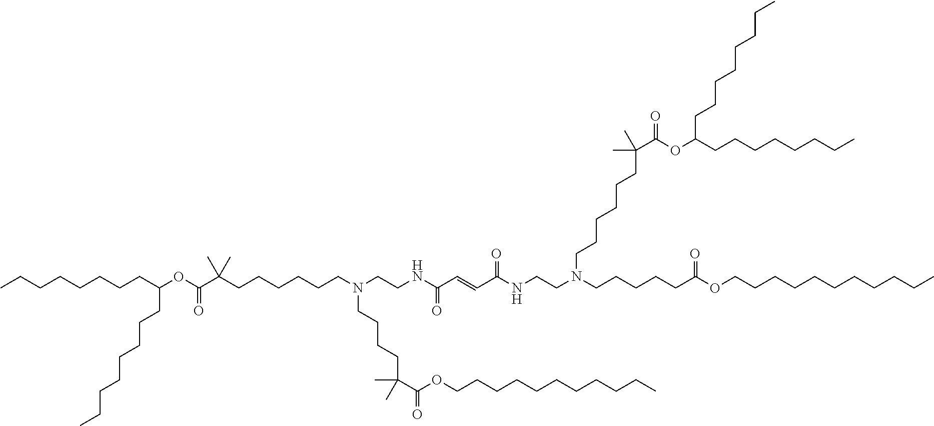 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) (E)-9-(5,5-dimethyl-6- oxo-6- (undecyloxy)hexyl)- 2,2,27,27-tetramethyl- 13,16-dioxo-20-(6- oxo-6- (undecyloxy)hexyl)- 9,12,17,20- tetraazaoctacos-14- enedioate |
| A102 | 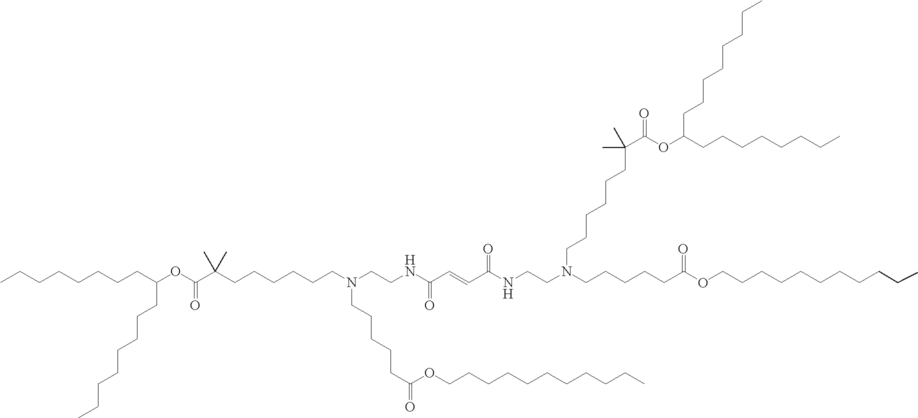 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] | di(heptadecan-9-yl) (E)-2,2,27,27- tetramethyl-13,16- dioxo-9,20-bis(6-oxo- 6-(undecyloxy)hexyl)- 9,12,17,20- tetraazaoctacos-14- enedioate |
| A103 | 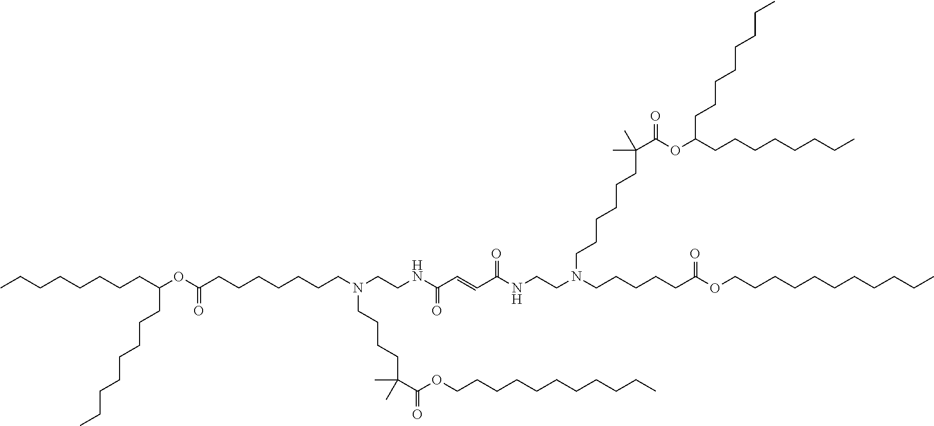 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | di(heptadecan-9-yl) (E)-20-(5,5-dimethyl-6- oxo-6- (undecyloxy)hexyl)- 2,2-dimethyl-13,16- dioxo-9-(6-oxo-6- (undecyloxy)hexyl)- 9,12,17,20- tetraazaoctacos-14- enedioate |
| A104 | 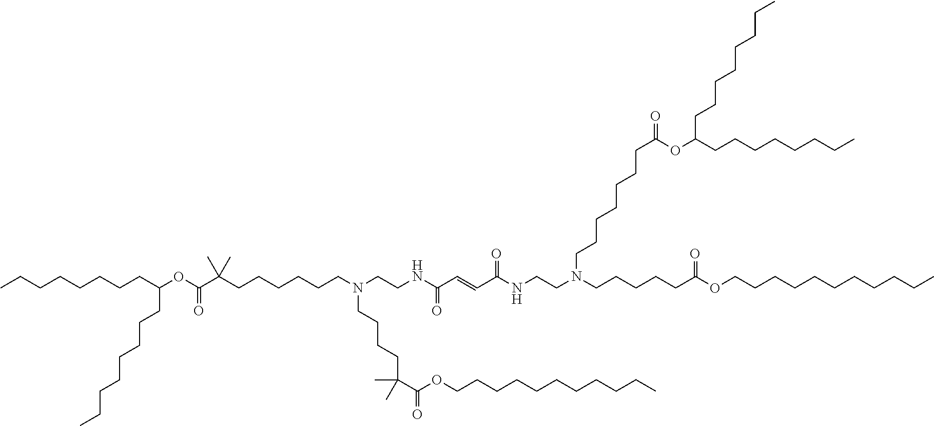 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) (E)-9-(5,5-dimethyl-6- oxo-6- (undecyloxy)hexyl)- 2,2-dimethyl-13,16- dioxo-20-(6-oxo-6- (undecyloxy)hexyl)- 9,12,17,20- tetraazaoctacos-14- enedioate |
| A105 | 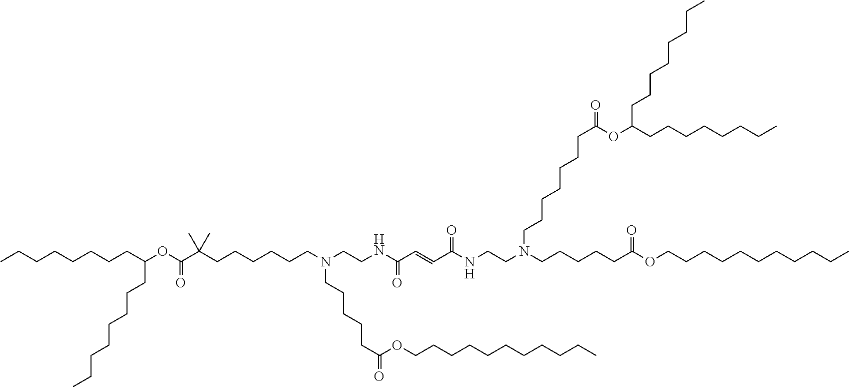 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] | di(heptadecan-9-yl) (E)-2,2-dimethyl- 13,16-dioxo-9,20- bis(6-oxo-6- (undecyloxy)hexyl)- 9,12,17,20- tetraazaoctacos-14- enedioate |
| A106 | 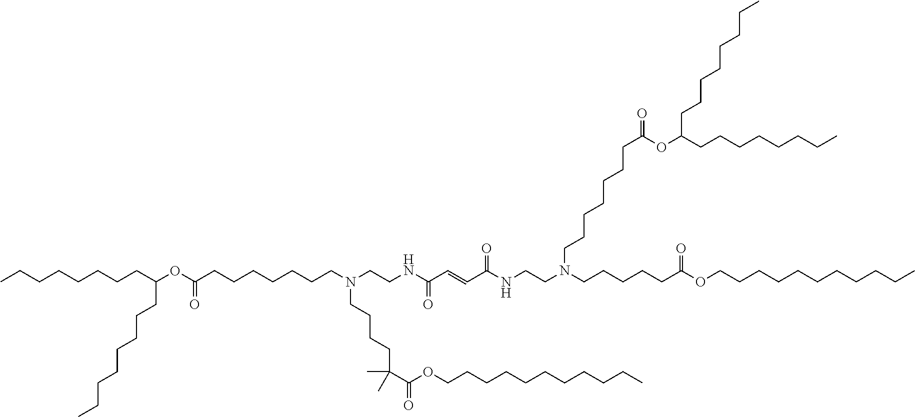 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | di(heptadecan-9-yl) (E)-9-(5,5-dimethyl-6- oxo-6- (undecyloxy)hexyl)- 13,16-dioxo-20-(6- oxo-6- (undecyloxy)hexyl)- 9,12,17,20- tetraazaoctacos-14- enedioate |
| B1 | 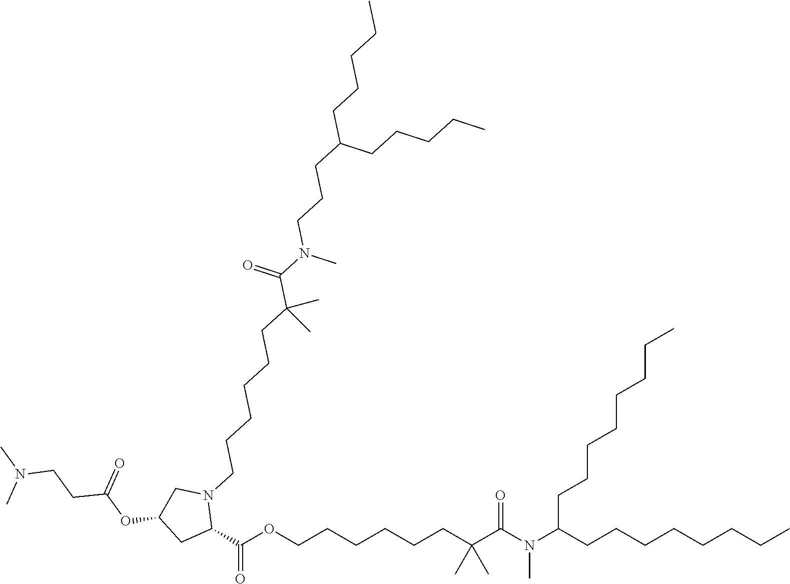 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCC(C)(C)C(=O)N(C)CCCC(CCCCC)CCCCC] | 8-(heptadecan-9- yl(methyl)amino)-7,7- dimethyl-8-oxooctyl (2S,4S)-1-(7,7- dimethyl-8-(methyl(4- pentylnonyl)amino)-8- oxooctyl)-4-((3- (dimethylamino)propa- noyl)oxy)pyrrolidine-2- carboxylate |
| B2 | 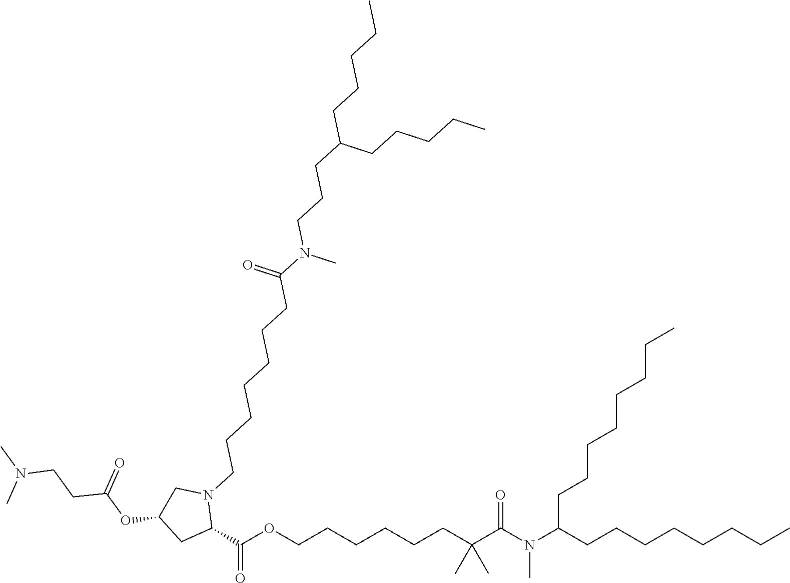 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCCC(=O)N(C)CCCC(CCCCC)CCCCC] | 8-(heptadecan-9- yl(methyl)amino)-7,7- dimethyl-8-oxooctyl (2S,4S)-4-((3- (dimethylamino)propa- noyl)oxy)-1-(8- (methyl(4- pentylnonyl)amino)-8- oxooctyl)pyrrolidine-2- carboxylate |
| B3 | 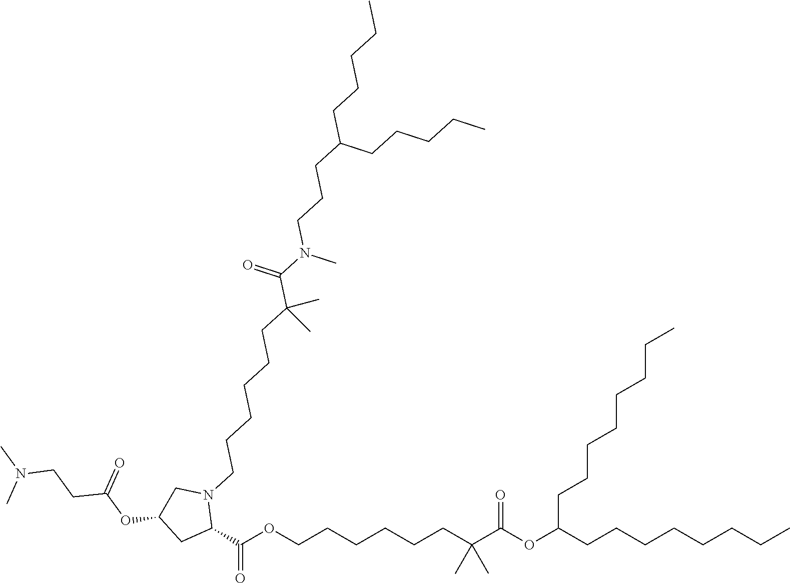 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)N(C)CCCC(CCCCC)CCCCC] | 8-(heptadecan-9- yloxy)-7,7-dimethyl-8- oxooctyl (2S,4S)-1- (7,7-dimethyl-8- (methyl(4- pentylnonyl)amino)-8- oxooctyl)-4-((3- (dimethylamino)propa- noyl)oxy)pyrrolidine-2- carboxylate |
| B4 | 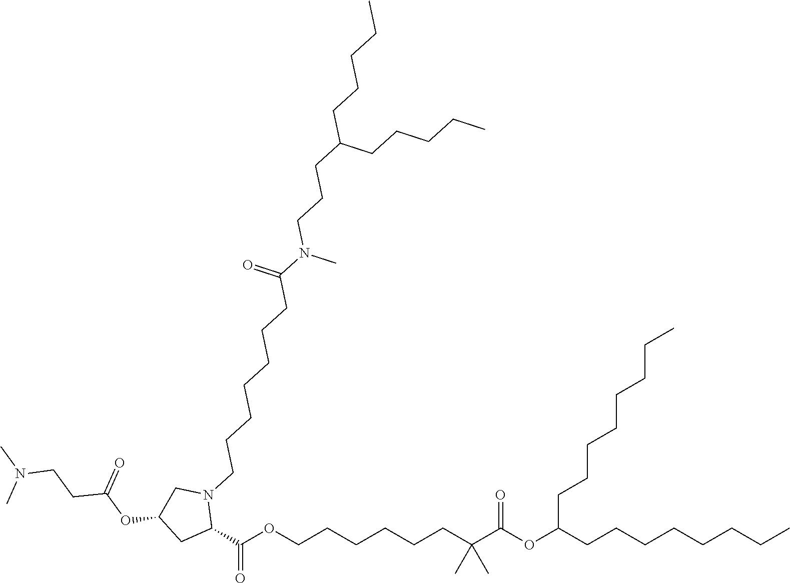 [SMILES: CCCCCCCCC(CCCCCCCC)OC(=O)C(C)(C)CCCCCCOC(=O)[C@@H]1C[C@H](OC(=O)CCN(C)C)CN1CCCCCCCC(=O)N(C)CCCC(CCCCC)CCCCC] | 8-(heptadecan-9- yloxy)-7,7-dimethyl-8- oxooctyl (2S,4S)-4-((3- (dimethylamino)propa- noyl)oxy)-1-(8- (methyl(4- pentylnonyl)amino)-8- oxooctyl)pyrrolidine-2- carboxylate |
| B5 | 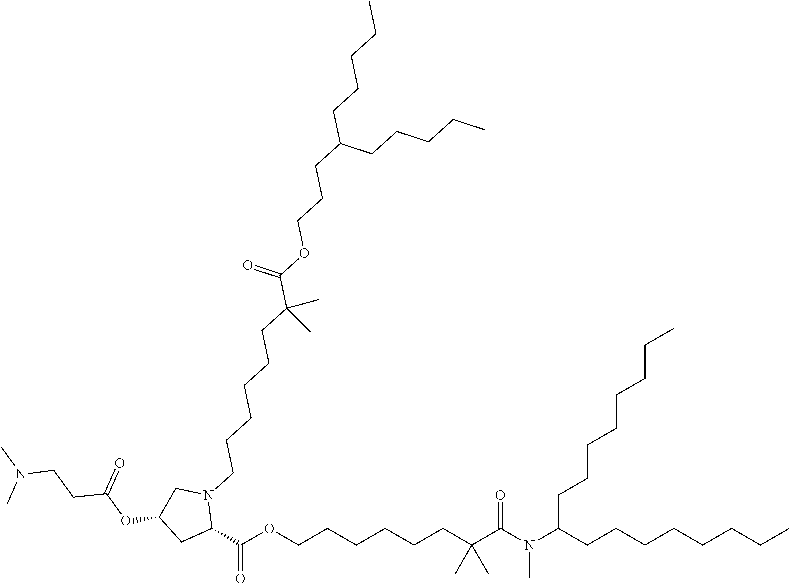 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | 8-(heptadecan-9- yl(methyl)amino)-7,7- dimethyl-8-oxooctyl (2S,4S)-1-(7,7- dimethyl-8-oxo-8-((4- pentylnonyl)oxy)octyl)- 4-((3- (dimethylamino)propa- noyl)oxy)pyrrolidine-2- carboxyalte |
| B6 | 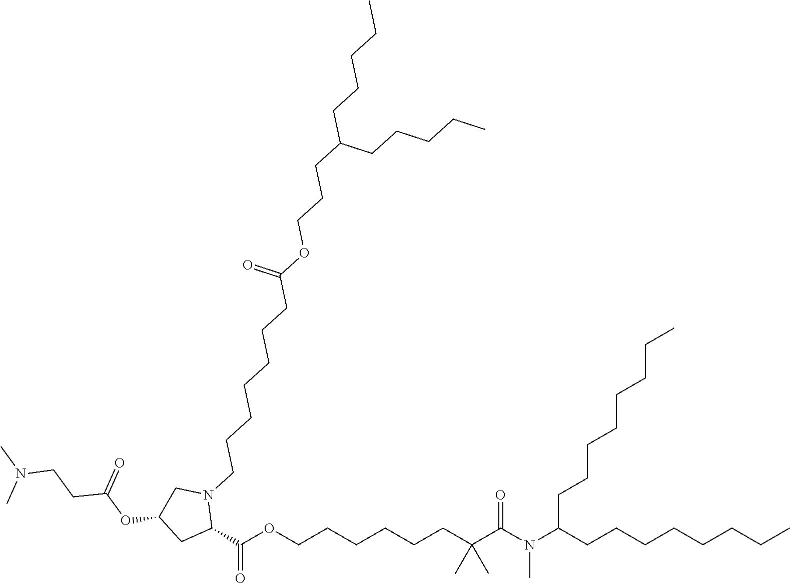 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCCC(=O)OCCCC(CCCCC)CCCCC] | 8-(heptadecan-9- yl(methyl)amino)-7,7- dimethyl-8-oxooctyl (2S,4S)-4-((3- (dimethylamino)propa- noyl)oxy)-1-(8-oxo-8- ((4- pentylnonyl)oxy)octyl) pyrrolidine-2- carboxylate |
| B7 | 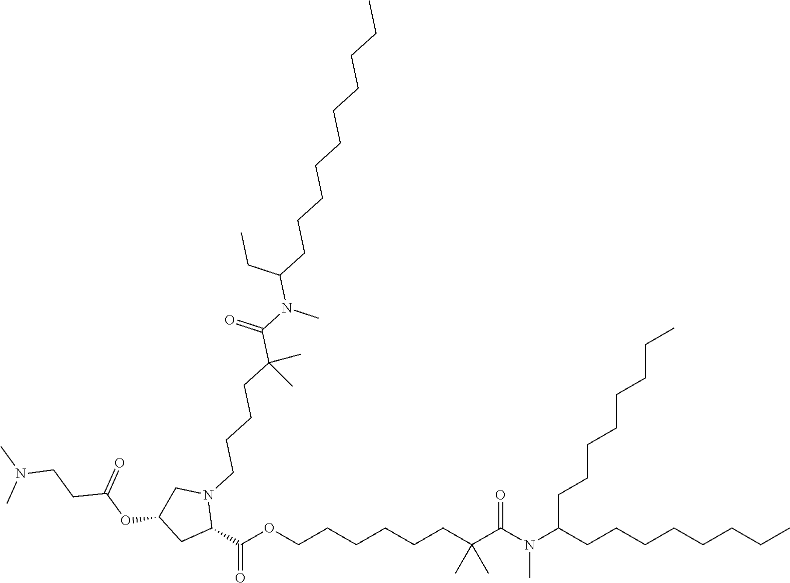 [SMILES: CCCCCCCCCCC(CC)N(C)C(=O)C(C)(C)CCCCN1C[C@@H](OC(=O)CCN(C)C)C[C@H]1C(=O)OCCCCCCC(C)(C)C(=O)N(C)C(CCCCCCCC)CCCCCCCC] | 8-(heptadecan-9- yl(methyl)amino)-7,7- dimethyl-8-oxooctyl (2S,4S)-1-(5,5- dimethyl-6- (methyl(tridecan-3- yl)amino)-6-oxohexyl)- 4-((3- (dimethylamino)propa- noyl)oxy)pyrrolidine-2- carboxylate |
| B8 | 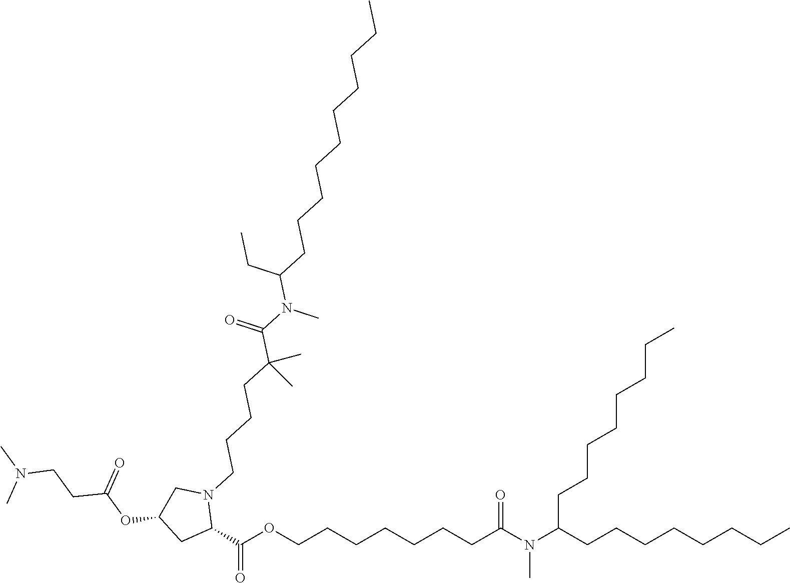 [SMILES: CCCCCCCCCCC(CC)N(C)C(=O)C(C)(C)CCCCN1C[C@@H](OC(=O)CCN(C)C)C[C@H]1C(=O)OCCCCCCCC(=O)N(C)C(CCCCCCCC)CCCCCCCC] | 8-(heptadecan-9- yl(methyl)amino)-8- oxooctyl (2S,4S)-1- (5,5-dimethyl-6- (methyl(tridecan-3- yl)amino)-6-oxohexyl)- 4-((3- (dimethylamino)propa- noyl)oxy)pyrroldiine-2- carboxylate |
| B9 | 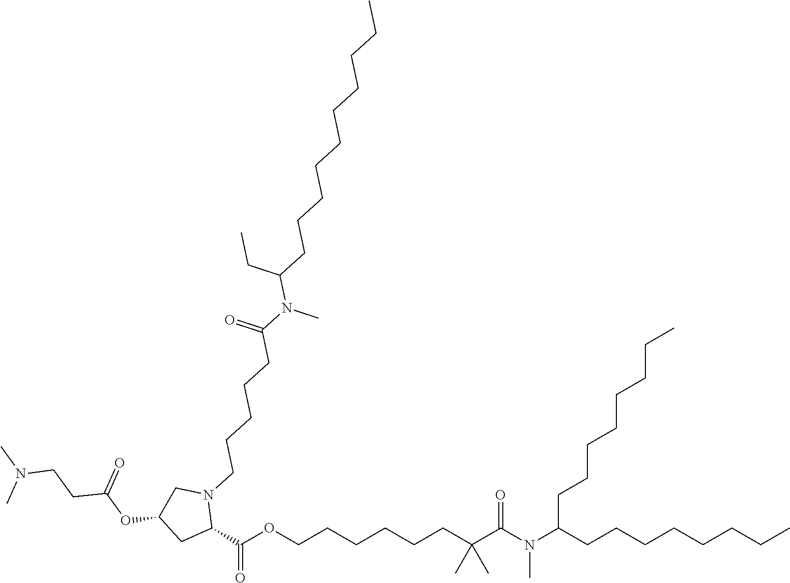 [SMILES: CCCCCCCCCCC(CC)N(C)C(=O)CCCCCN1C[C@@H](OC(=O)CCN(C)C)C[C@H]1C(=O)OCCCCCCC(C)(C)C(=O)N(C)C(CCCCCCCC)CCCCCCCC] | 8-(heptadecan-9- yl(methyl)amino)-7,7- dimethyl-8-oxooctyl (2S,4S)-4-((3- (dimethylamino)propa- noyl)oxy)-1-(6- (methyl(tridecan-3- yl)amino)-6- oxohexyl)pyrrolidine-2- carboxylate |
| B10 | 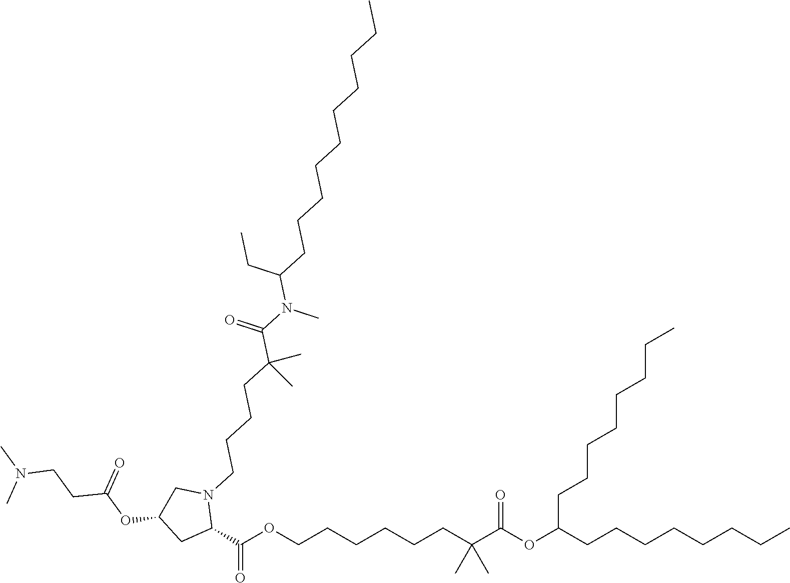 [SMILES: CCCCCCCCCCC(CC)N(C)C(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl-8- oxooctyl (2S,4S)-1- (5,5-dimethyl-6- (methyl(tridecan-3- yl)amino)-6-oxohexyl)- 4-((3- (dimethylamino)propa- noyl)oxy)pyrrolidine-2- carboxylate |
| B11 | 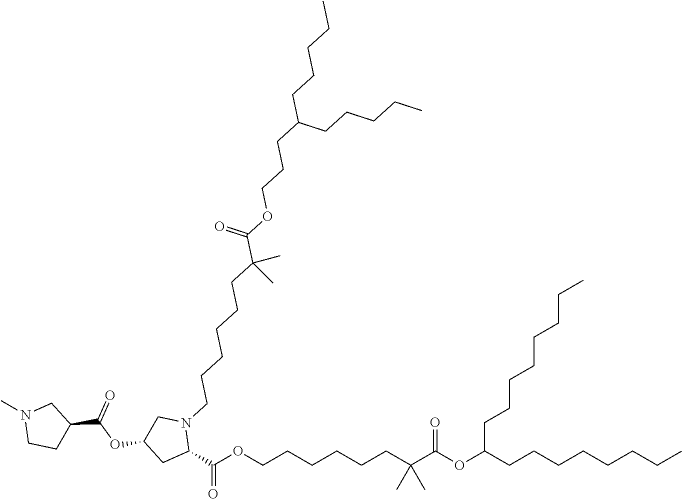 [SMILES: CCCCCCCCC(CCCCCCCC)OC(=O)C(C)(C)CCCCCCOC(=O)[C@@H]1C[C@H](OC(=O)[C@H]2CCN(C)C2)CN1CCCCCCC(C)(C)C(=O)OCCCC(CCCCC)CCCCC] | 8-(heptadecan-9- yloxy)-7,7-dimethyl-8- oxooctyl (2S,4S)-1- (7,7-dimethyl-8-oxo-8- ((4- pentylnonyl)oxy)octyl)- 4-(((R)-1- methylpyrrolidine-3- carbonyl)oxy)pyrrolidine- 2-carboxylate |
| B12 | 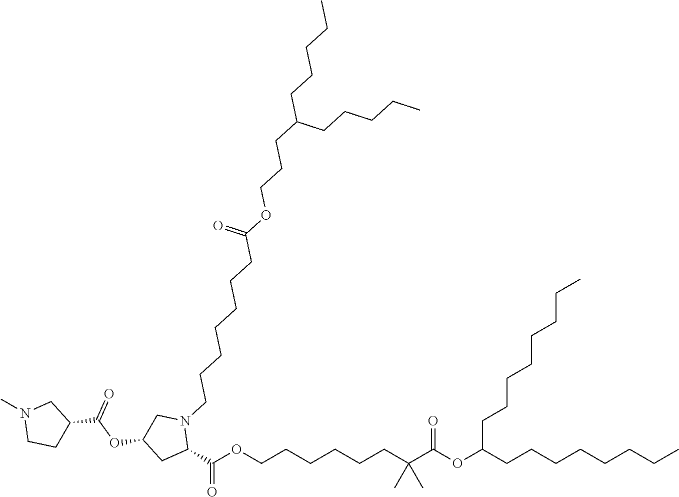 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCCC(=O)OCCCC(CCCCC)CCCCC] | 8-(heptadecan-9- yloxy)-7,7-dimethyl-8- oxooctyl (2S,4S)-4- (((R)-1- methylpyrrolidine-3- carbonyl)oxy)-1-(8- oxo-8-((4- pentylnonyl)oxy)octyl) pyrrolidine-2- carboxylate |
| B13 | 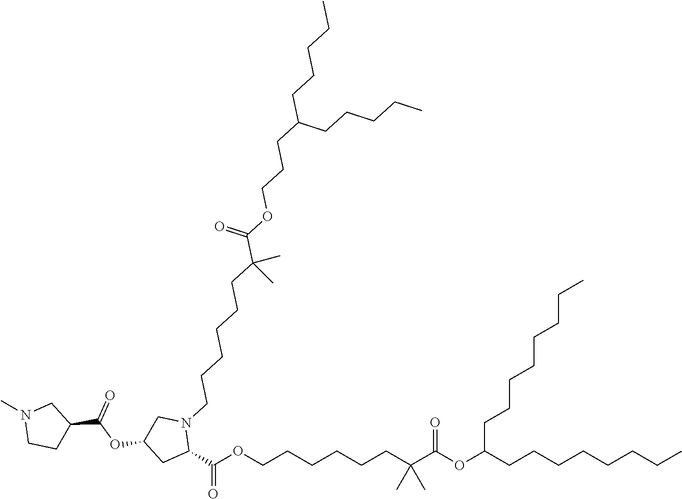 [SMILES: CCCCCCCCC(CCCCCCCC)OC(=O)C(C)(C)CCCCCCOC(=O)[C@@H]1C[C@H](OC(=O)[C@H]2CCN(C)C2)CN1CCCCCCC(C)(C)C(=O)OCCCC(CCCCC)CCCCC] | 8-(heptadecan-9- yloxy)-7,7-dimethyl-8- oxooctyl (2S,4S)-1- (7,7-dimethyl-8-oxo-8- ((4- pentylnonyl)oxy)octyl)- 4-(((S)-1- methylpyrrolidine-3- carbonyl)oxy)pyrrolidine- 2-carboxylate |
| B14 | 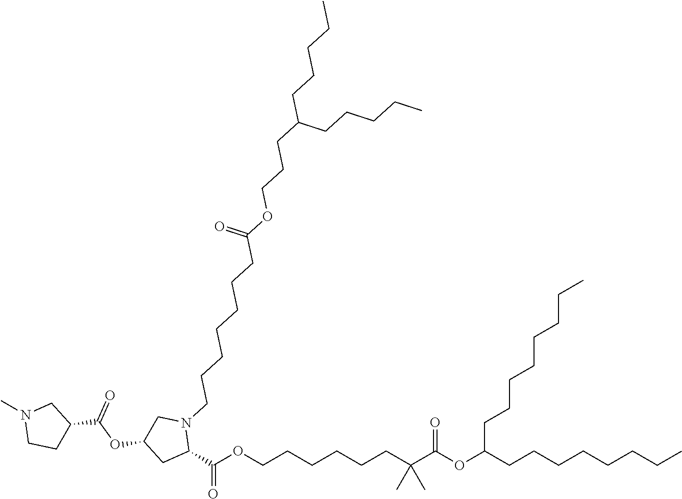 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCCC(=O)OCCCC(CCCCC)CCCCC] | 8-(heptadecan-9- yloxy)-7,7-dimethyl-8- oxooctyl (2S,4S)-4- (((S)-1- methylpyrrolidine-3- carbonyl)oxy)-1-(8- oxo-8-((4- pentylnonyl)oxy)octyl) pyrrolidine-2- carboxylate |
| B15 | 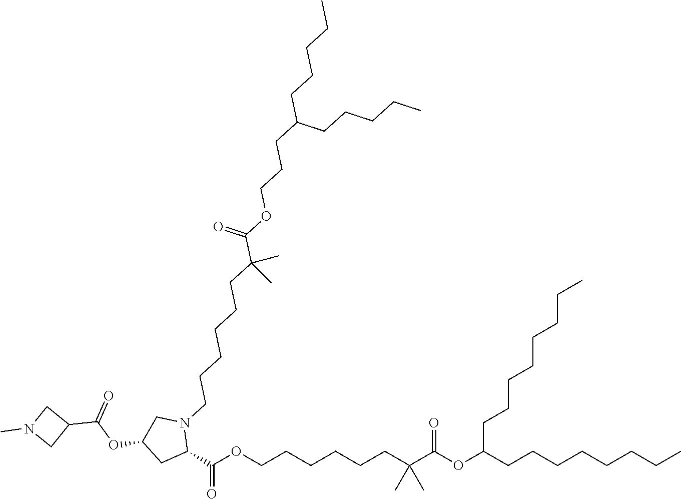 [SMILES: CCCCCCCCC(CCCCCCCC)OC(=O)C(C)(C)CCCCCCOC(=O)[C@@H]1C[C@H](OC(=O)C2CN(C)C2)CN1CCCCCCC(C)(C)C(=O)OCCCC(CCCCC)CCCCC] | 8-(heptadecan-9- yloxy)-7,7-dimethyl-8- oxooctyl (2S,4S)-1- (7,7-dimethyl-8-oxo-8- ((4- plentylnonyl)oxy)octyl)- 4-((1-methylazetidine- 3- carbonyl)oxy)pyrrolidine- 2-carboxylate |
| B16 | 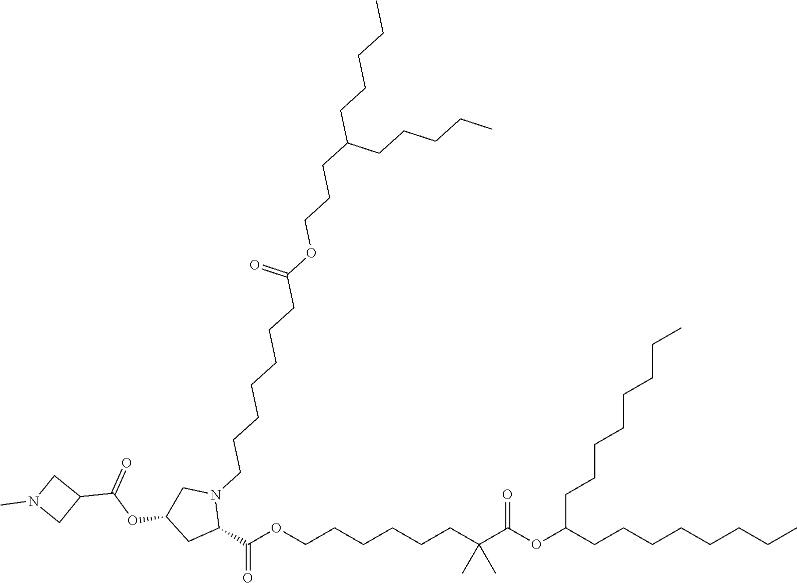 [SMILES: CCCCCCCCC(CCCCCCCC)OC(=O)C(C)(C)CCCCCCOC(=O)[C@@H]1C[C@H](OC(=O)C2CN(C)C2)CN1CCCCCCCC(=O)OCCCC(CCCCC)CCCCC] | 8-(heptadecan-9- yloxy)-7,7-dimethyl-8- oxooctyl (2S,4S)-4-((1- methylazetidine-3- carbonyl)oxy)-1-(8- oxo-8-((4- pentylnonyl)oxy)octyl) pyrrolidine-2- carboxylate |
| B17 | 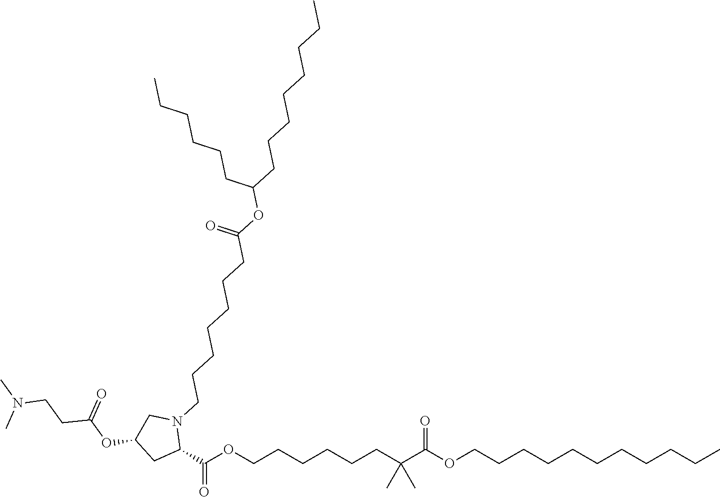 [SMILES: CCCCCCCCCCCOC(=O)C(C)(C)CCCCCCOC(=O)[C@@H]1C[C@H](OC(=O)CCN(C)C)CN1CCCCCCCC(=O)OC(CCCCCC)CCCCCCCC] | 7,7-dimethyl-8-oxo-8- (undecyloxy)octyl (2S,4)S-4-((3- (dimethylamino)propa- noyl)oxy)-1-(8-oxo-8- (pentadecan-7- yloxy)octyl)pyrrolidine- 2-carboxylate |
| B18 | 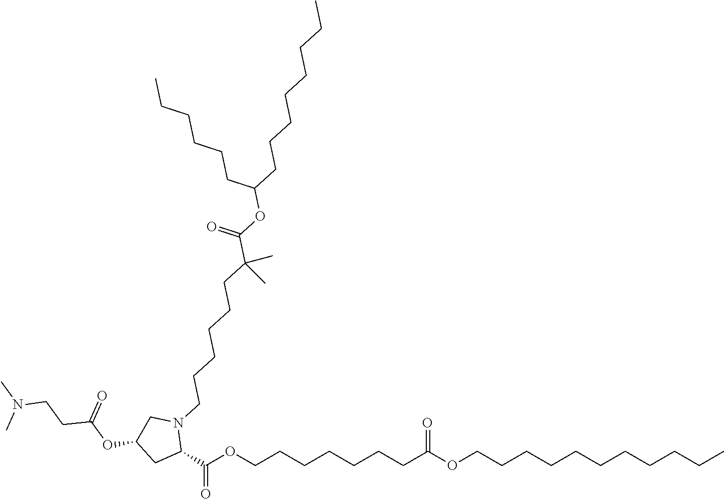 [SMILES: CCCCCCCCCCCOC(=O)CCCCCCCOC(=O)[C@@H]1C[C@H](OC(=O)CCN(C)C)CN1CCCCCCC(C)(C)C(=O)OC(CCCCCC)CCCCCCCC] | 8-oxo-8- (undecyloxy)octyl (2S,4S)-1-(7,7- dimethyl-8-oxo-8- (pentadecan-7- yloxy)octyl)-4-((3- (dimethylamino)propa- noyl)oxy)pyrrolidine-2- carboxylate |
| B19 | 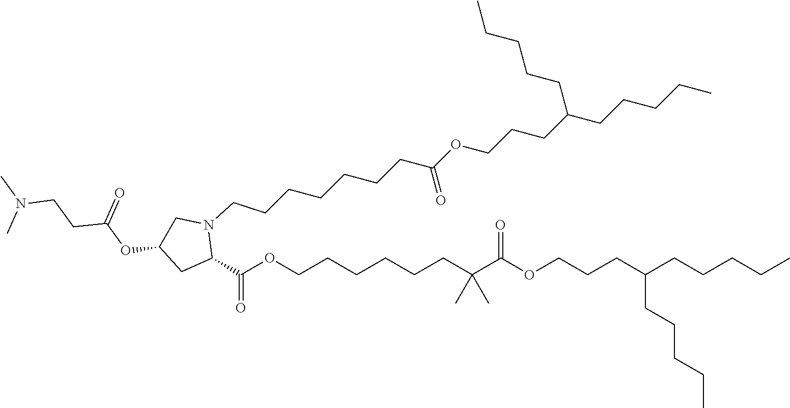 [SMILES: CCCCCC(CCCCC)CCCOC(=O)CCCCCCCN1C[C@@H](OC(=O)CCN(C)C)C[C@H]1C(=O)OCCCCCCC(C)(C)C(=O)OCCCC(CCCCC)CCCCC] | 7,7-dimethyl-8-oxo-8- ((4- pentylnonyl)oxy)octyl (2S,4)S-4-((3- (dimethylamino)propa- noyl)oxy)-1-(8-oxo-8- ((4- pentylnonyl)oxy)octyl) pyrroldiine-2- carboxylate |
| B20 | 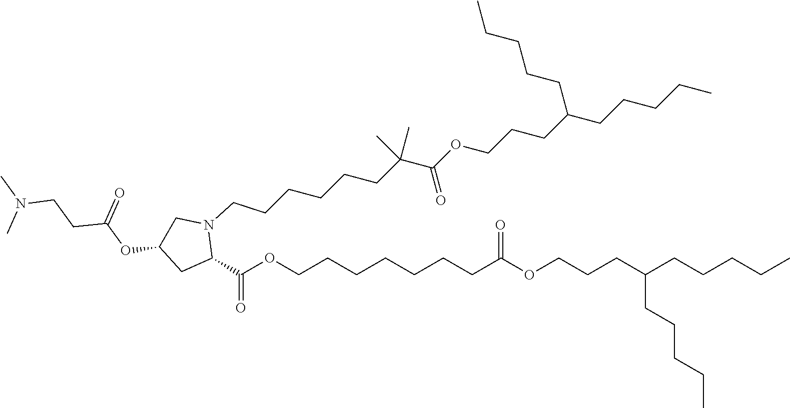 [SMILES: CCCCCC(CCCCC)CCCOC(=O)CCCCCCCOC(=O)[C@@H]1C[C@H](OC(=O)CCN(C)C)CN1CCCCCCC(C)(C)C(=O)OCCCC(CCCCC)CCCCC] | 8-oxo-8-((4- pentylnonyl)oxy)octyl (2S,4S)-1-(7,7- dimethyl-8-oxo-8-((4- pentylnonyl)oxy)octyl)- 4-((3- (dimethylamino)propa- noyl)oxy)pyrrolidine-2- carboxylate |
| B21 | 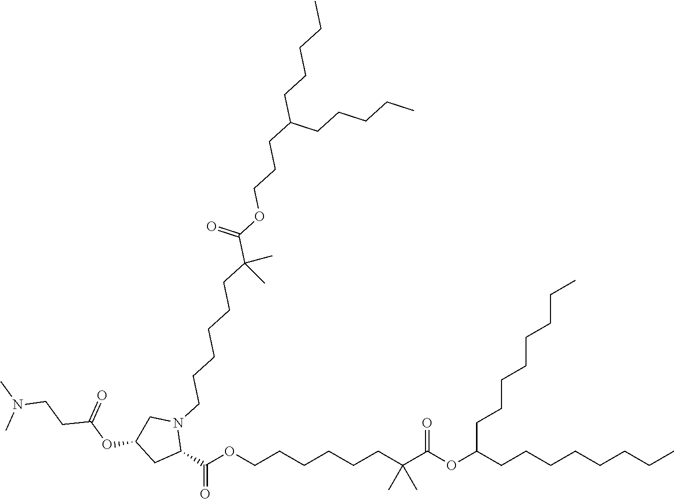 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | 8-(heptadecan-9- yloxy)-7,7-dimethyl-8- oxooctyl (2S,4R)-1- (7,7-dimethyl-8-oxo-8- ((4- pentylnonyl)oxy)octyl)- 4-((3- (dimethylamino)propa- noyl)oxy)pyrrolidine-2- carboxylate |
| B22 | 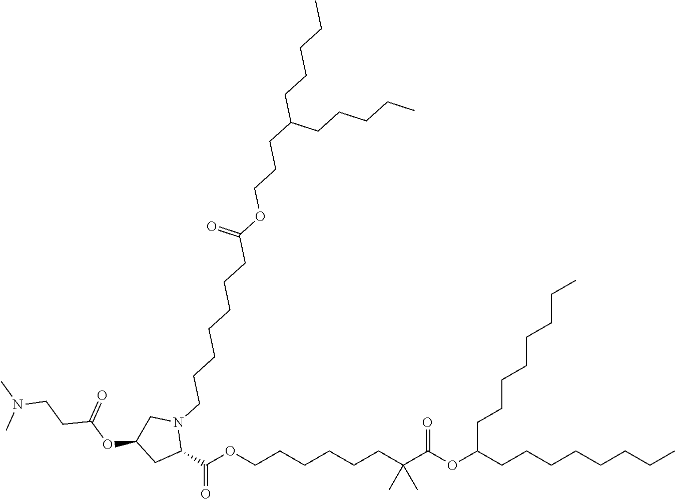 [SMILES: CCCCCCCCC(CCCCCCCC)OC(=O)C(C)(C)CCCCCCOC(=O)[C@@H]1C[C@@H](OC(=O)CCN(C)C)CN1CCCCCCCC(=O)OCCCC(CCCCC)CCCCC] | 8-(heptadecan-9- yloxy)-7,7-dimethyl-8- oxooctyl (2S,4R)-4-((3- (dimethylamino)propa- noyl)oxy)-1-(8-oxo-8- ((4- pentylnonyl)oxy)octyl) pyrrolidine-2- carboxylate |
| B23 | 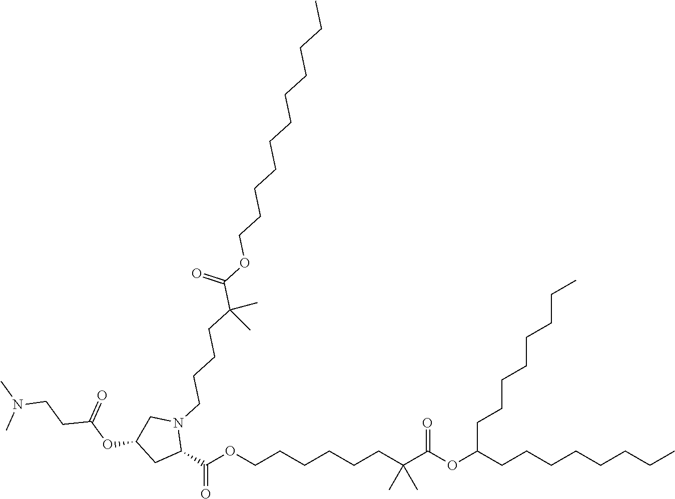 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl-8- oxooctyl (2S,4S)-1- (5,5-dimethyl-6-oxo-6- (undecyloxy)hexyl)-4- ((3- (dimethylamino)propa- noyl)oxy)pyrrolidine-2- carboxylate |
| B24 | 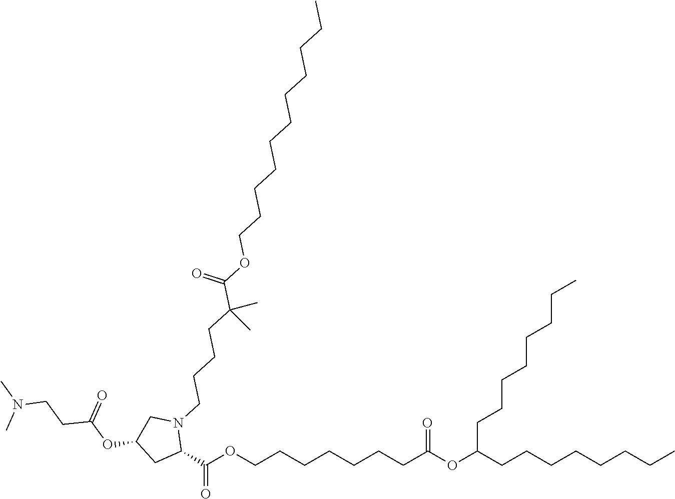 [SMILES: CCCCCCCCCCCOC(=O)C(C)(C)CCCCN1C[C@@H](OC(=O)CCN(C)C)C[C@H]1C(=O)OCCCCCCCC(=O)OC(CCCCCCCC)CCCCCCCC] | 8-(heptadecan-9- yloxy)-8-oxooctyl (2S,4S)-1-(5,5- dimethyl-6-oxo-6- (undecyloxy)hexyl)-4- ((3- (dimethylamino)propa- noyl)oxy)pyrrolidine-2- carboxylate |
| B25 | 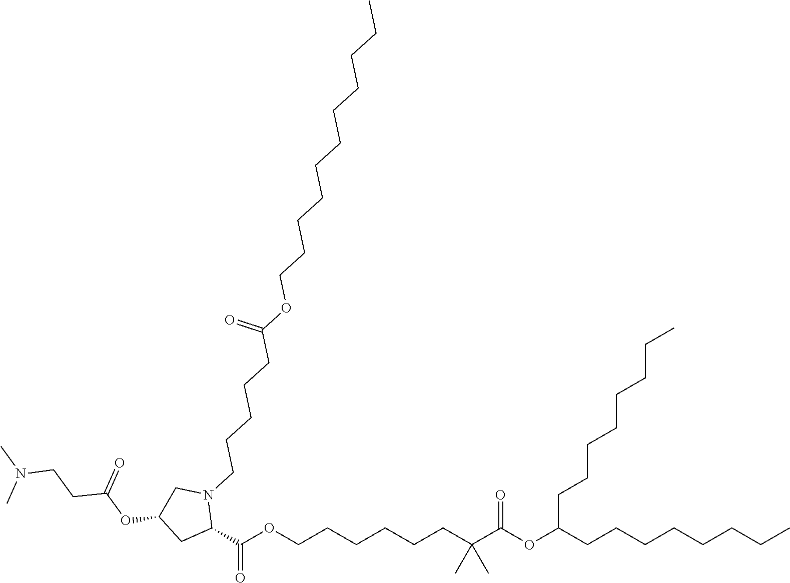 [SMILES: CCCCCCCCCCCOC(=O)CCCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl-8- oxooctyl (2S,4S)-4-((3- (dimethylamino)propa- noyl)oxy)-1-(6-oxo-6- (undecyloxy)hexyl)pyrro- lidine-2-carboxylate |

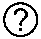

In some embodiments, a lipid membrane of the LNMPs comprises at least 35% of the lipid compound from group i), e.g., at least 40%, 45%, 50%, 55%, 60%, 65%, 70%, 75% 80%, 85%, 90%, or more than 90% of the lipid compound from group i), e.g., 35%-40%, 40%-50%, 50%-60%, 60%-70%, 70%-80%, or 80%-90% of the lipid compound from group i).

In some embodiments, a lipid membrane of the LNMPs comprises at least 35% of the lipid compound from group ii), e.g., at least 40%, 45%, 50%, 55%, 60%, 65%, 70%, 75% 80%, 85%, 90%, or more than 90% of the lipid compound from group ii), e.g., 35%-40%, 40%-50%, 50%-60%, 60%-70%, 70%-80%, or 80%-90% of the lipid compound from group ii).

In some embodiments, a lipid membrane of the LNMPs comprises at least 35% of the lipid compound from group iii), e.g., at least 40%, 45%, 50%, 55%, 60%, 65%, 70%, 75% 80%, 85%, 90%, or more than 90% of the lipid compound from group iii), e.g., 35%-40%, 40%-50%, 50%-60%, 60%-70%, 70%-80%, or 80%-90% of the lipid compound from group iii).

In some embodiments, a lipid membrane of the LNMPs comprises at least 35% of the lipid compound from group iii), e.g., at least 40%, 45%, 50%, 55%, 60%, 65%, 70%, 75% 80%, 85%, 90%, or more than 90% of the lipid compound from group iii), e.g., 35%-40%, 40%-50%, 50%-60%, 60%-70%, 70%-80%, or 80%-90% of the lipid compound from group iv).

In some instances, the LNMPs comprise at least 1%, 5%, 10%, 20%, 30%, 40%, 50%, 60%, 70%, 80%, 90%, or more than 90% ionizable lipid.

In some instances, the LNMPs comprise a molar ratio of at least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 25%-75% ionizable lipid (e.g., about 25%-75% ionizable lipid).

### Other Ionizable Lipids

In the LNMP formulations, more than one ionizable lipid can be used for the ionizable lipid component: one or more of the ionizable lipids from the compounds of formulas in groups i)-iv) can be used alone or in combination with a different ionizable lipid from the compounds of formulas in groups i)-iv).

In some embodiments, the ionizable lipid do not include 1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5 (Moderna), and 98N12-5.

In some embodiments, the additional ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

In some embodiments, the additional ionizable lipid is represented by the following formula III:

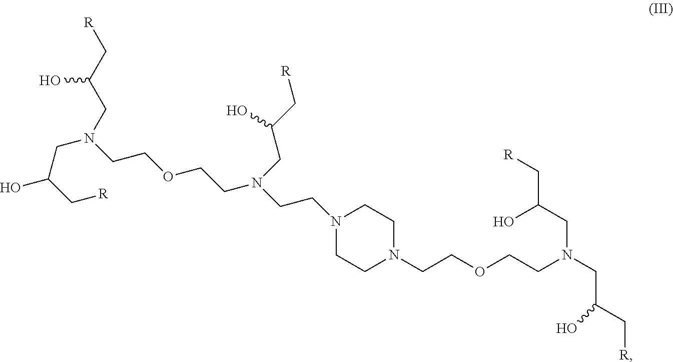

wherein R is C₈-C₁₄ alkyl group.

The ionizable lipid described herein may include an amine core described herein substituted with one or more (e.g., 1, 2, 3, 4, 5, or 6) lipid tails. In some embodiments, the ionizable lipid described herein include at least 3 lipid tails. A lipid tail may be a C₈-C₁₈ hydrocarbon (e.g., C₆-C₁₈ alkyl or C₆-C₁₈ alkanoyl). An amine core may be substituted with one or more lipid tails at a nitrogen atom (e.g., one hydrogen atom attached to the nitrogen atom may be replaced with a lipid tail).

In some embodiments, the amine core has a structure of:

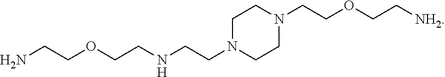 [SMILES: NCCOCCNCCN1CCN(CCOCCN)CC1]

In some embodiments, the amine core has a structure of:

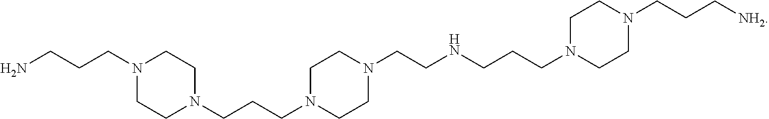 [SMILES: NCCCN1CCN(CCCNCCN2CCN(CCCN3CCN(CCCN)CC3)CC2)CC1]

In some embodiments, the amine core has a structure of:

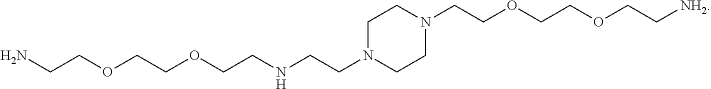 [SMILES: NCCOCCOCCNCCN1CCN(CCOCCOCCN)CC1]

In some embodiments, the amine core has a structure of:

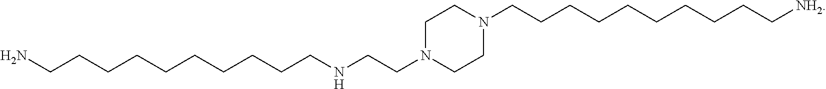 [SMILES: NCCCCCCCCCCNCCN1CCN(CCCCCCCCCCN)CC1]

In some embodiments, the amine core has a structure of:

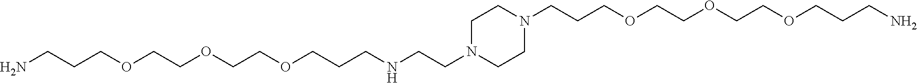 [SMILES: NCCCOCCOCCOCCCNCCN1CCN(CCCOCCOCCOCCCN)CC1]

In some embodiments, the amine core has a structure of:

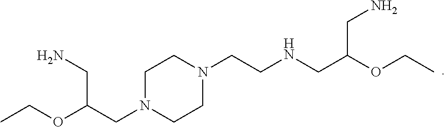 [SMILES: CCOC(CN)CNCCN1CCN(CC(CN)OCC)CC1]

In some embodiments, the amine core has a structure of:

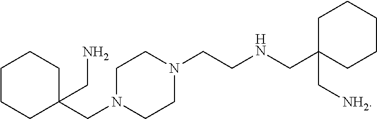 [SMILES: NCC1(CNCCN2CCN(CC3(CN)CCCCC3)CC2)CCCCC1]

In some embodiments, the amine core has a structure of:

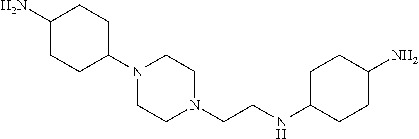 [SMILES: NC1CCC(NCCN2CCN(C3CCC(N)CC3)CC2)CC1]

Other suitable lipids for use in the RNA composition (e.g., the mRNA composition or the circRNA composition) and methods for making and using thereof include the lipids as described in International Patent Publication WO 2016/118725, which is incorporated herein by reference in its entirety.

In certain embodiments, the RNA composition (e.g., the mRNA therapeutic composition or the circRNA composition) and methods for making and using thereof include a lipid having a compound structure of:

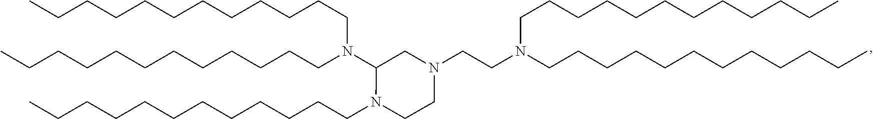 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable lipids for use in the RNA composition (e.g., the mRNA composition or the circRNA composition) and methods for making and using thereof include the lipids as described in International Patent Publication WO 2016/118724, which is incorporated herein by reference in its entirety.

In certain embodiments, the RNA composition (e.g., the mRNA composition or the circRNA composition) and methods for making and using thereof include a lipid having a compound structure of:

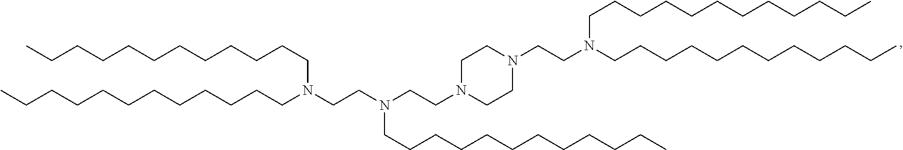 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable lipids for use in the RNA composition (e.g., the mRNA composition or the circRNA composition) and methods for making and using thereof include a lipid having the formula of 14,25-ditridecyl 15,18,21,24-tetraaza-octatriacontane, and pharmaceutically acceptable salts thereof.

Other suitable lipids for use in the RNA composition (e.g., the mRNA composition or the circRNA composition) and methods for making and using thereof include the lipids as described in International Patent Publications WO 2013/063468 and WO 2016/205691, each of which is incorporated herein by reference in its entirety.

In some embodiments, the RNA composition (e.g., the mRNA composition or the circRNA composition) and methods for making and using thereof include a lipid of the following formula:

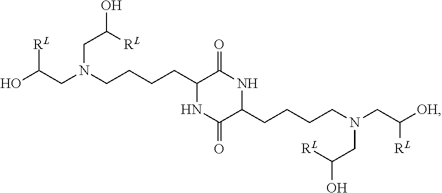

or pharmaceutically acceptable salts thereof, wherein each instance of R^{L} is independently optionally substituted C6-C40 alkenyl.

In certain embodiments, the RNA composition (e.g., the mRNA composition or the circRNA composition) and methods for making and using thereof include a lipid having a compound structure of:

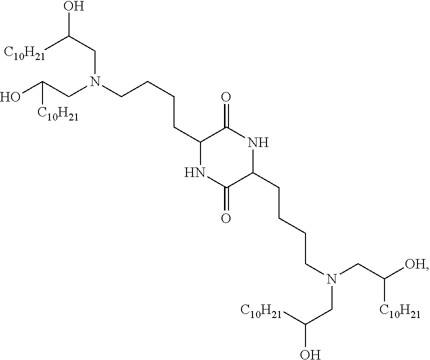

and pharmaceutically acceptable salts thereof.

In certain embodiments, the RNA composition (e.g., the mRNA composition or the circRNA composition) and methods for making and using thereof include a lipid having a compound structure of:

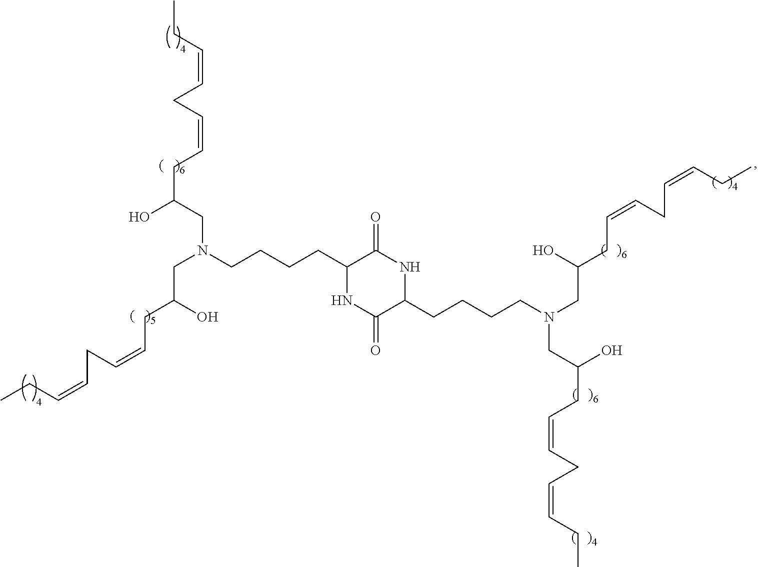

and pharmaceutically acceptable salts thereof.

In certain embodiments, the RNA composition (e.g., the mRNA composition or the circRNA composition) and methods for making and using thereof include a lipid having a compound structure of:

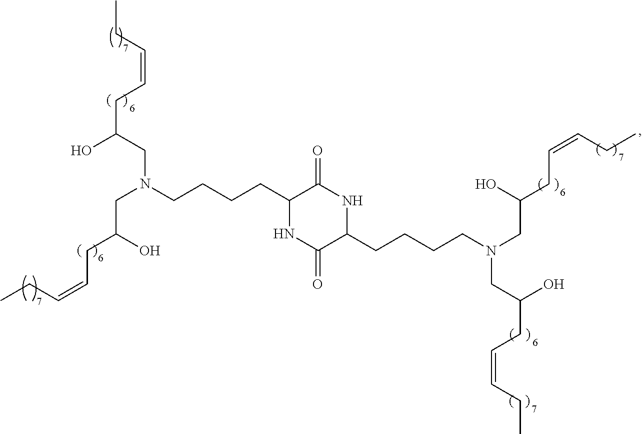

and pharmaceutically acceptable salts thereof.

In certain embodiments, the RNA composition (e.g., the mRNA composition or the circRNA composition) and methods for making and using thereof include a lipid having a compound structure of:

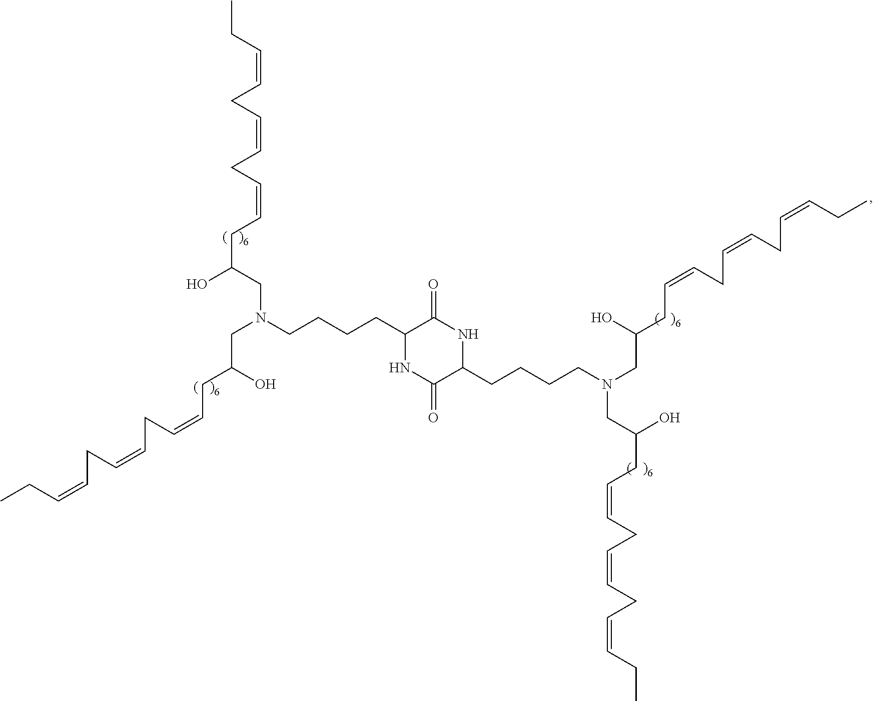

and pharmaceutically acceptable salts thereof.

Other suitable lipids for use in the RNA composition (e.g., the mRNA composition or the circRNA composition) and methods for making and using thereof include the lipids as described in International Patent Publication WO 2015/184256, which is incorporated herein by reference in its entirety. In some embodiments, the RNA composition (e.g., the mRNA composition) and methods for making and using thereof include a lipid of the following formula:

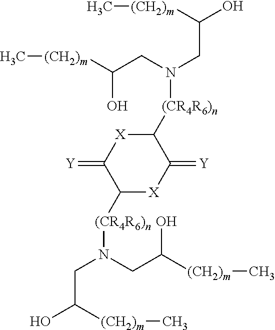

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen.

In certain embodiments, the RNA composition (e.g., the mRNA composition or the circRNA composition) and methods for making and using thereof include a lipid, “Target 23”, having a compound structure of:

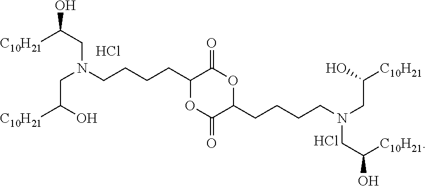

(Target 23) and pharmaceutically acceptable salts thereof.

Other suitable lipids for use in the RNA composition (e.g., the mRNA composition or the circRNA composition) and methods for making and using thereof include the lipids as described in International Patent Publication WO 2016/004202, which is incorporated herein by reference in its entirety.

In some embodiments, the RNA composition (e.g., the mRNA composition or the circRNA composition) and methods for making and using thereof include a lipid having the compound structure:

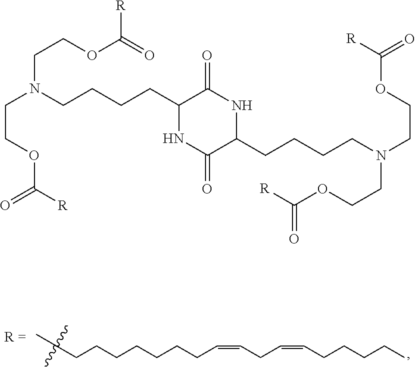

or a pharmaceutically acceptable salt thereof.

In some embodiments, the RNA composition (e.g., the mRNA composition or the circRNA composition) and methods for making and using thereof include a lipid having the compound structure:

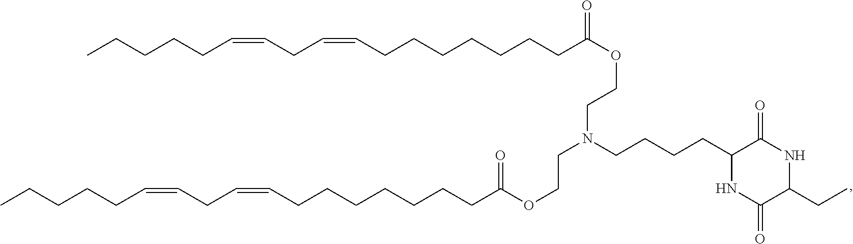 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof.

In some embodiments, the RNA composition (e.g., the mRNA composition or the circRNA composition) and methods for making and using thereof include a lipid having the compound structure:

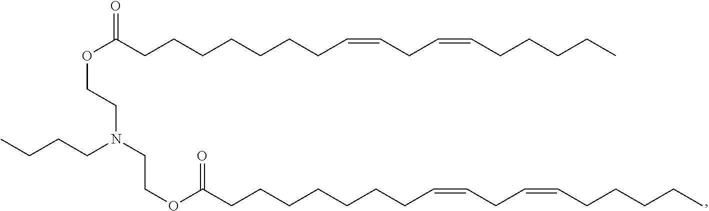 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable lipids for use in the RNA composition (e.g., the mRNA composition or the circRNA composition) and methods for making and using thereof include lipids as described in U.S. Provisional Patent Application Ser. No. 62/758,179, which is incorporated herein by reference in its entirety.

In some embodiments, the RNA composition (e.g., the mRNA composition or the circRNA composition) and methods for making and using thereof include a lipid of the following formula:

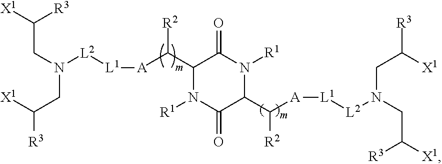

or a pharmaceutically acceptable salt thereof, wherein each R¹ and R² is independently H or C1-C6 aliphatic; each m is independently an integer having a value of 1 to 4; each A is independently a covalent bond or arylene; each L¹ is independently an ester, thioester, disulfide, or anhydride group; each L² is independently C2-C10 aliphatic; each X¹ is independently H or OH; and each R³ is independently C6-C20 aliphatic.

In some embodiments, the RNA composition (e.g., the mRNA composition or the circRNA composition) and methods for making and using thereof include a lipid of the following formula:

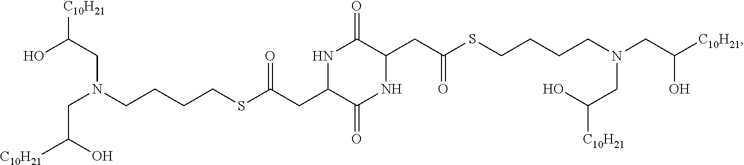

or a pharmaceutically acceptable salt thereof.

In some embodiments, the RNA composition (e.g., the mRNA composition or the circRNA composition) and methods for making and using thereof include a lipid of the following formula:

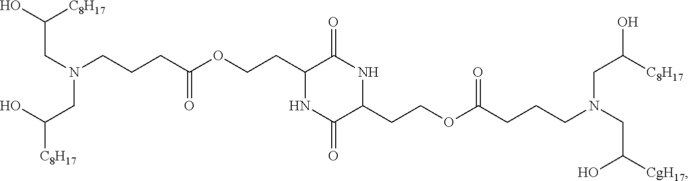

or a pharmaceutically acceptable salt thereof.

In some embodiments, the RNA composition (e.g., the mRNA composition or the circRNA composition) and methods for making and using thereof include a lipid of the following formula:

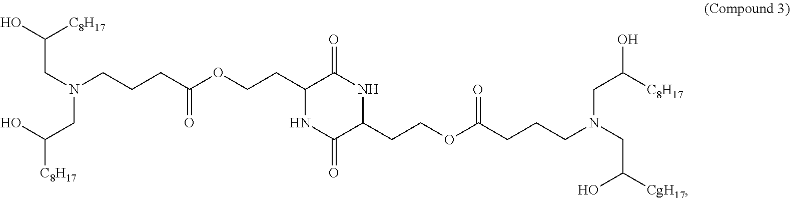

or a pharmaceutically acceptable salt thereof.

Other suitable lipids for use in the RNA composition (e.g., the mRNA composition or the circRNA composition) and methods for making and using thereof include the lipids as described in J. McClellan, M. C. King, Cell 2010, 141, 210-217 and in Whitehead et al., Nature Communications (2014) 5:4277, which is incorporated herein by reference in its entirety.

In certain embodiments, the lipids of the RNA composition (e.g., the mRNA composition or the circRNA composition) and methods for making and using thereof include a lipid having a compound structure of:

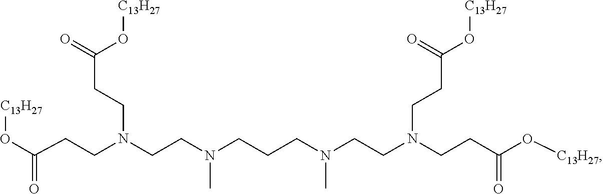

and pharmaceutically acceptable salts thereof.

Other suitable lipids for use in the RNA composition (e.g., the mRNA composition or the circRNA composition) and methods for making and using thereof include the lipids as described in International Patent Publication WO 2015/199952, which is incorporated herein by reference in its entirety.

In some embodiments, the RNA composition (e.g., the mRNA composition or the circRNA composition) and methods for making and using thereof include a lipid having the compound structure:

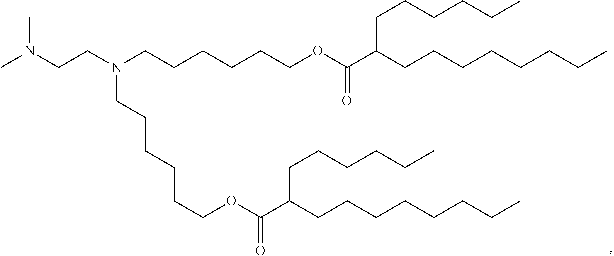 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

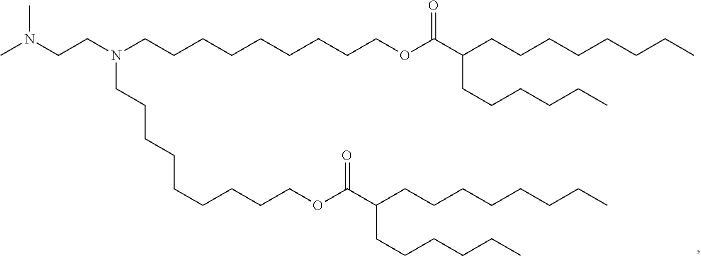 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

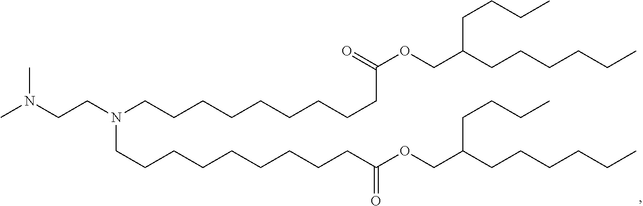 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

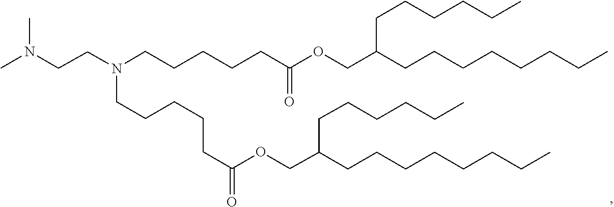 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

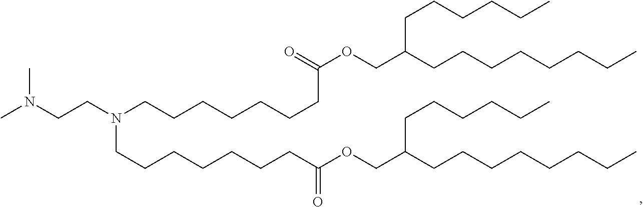 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

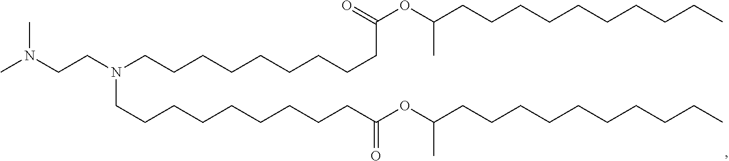 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

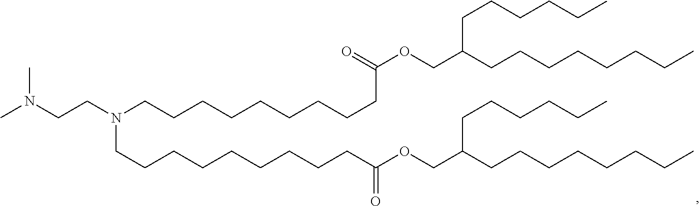 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

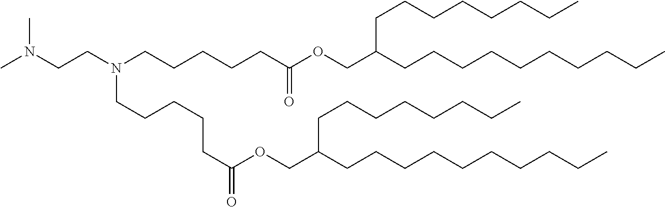 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

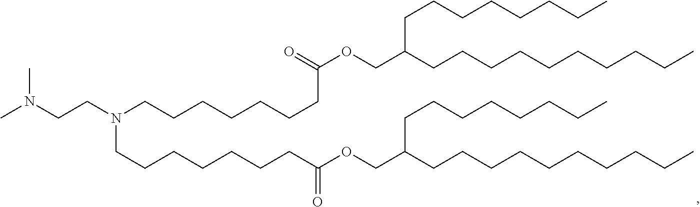 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

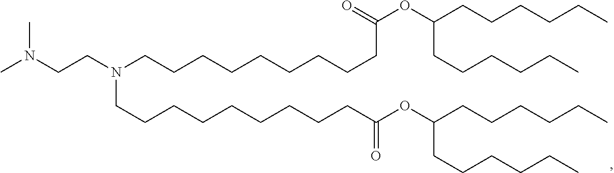 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof.

In some embodiments, the RNA composition (e.g., the mRNA composition or the circRNA composition) and methods for making and using thereof include a lipid having the compound structure:

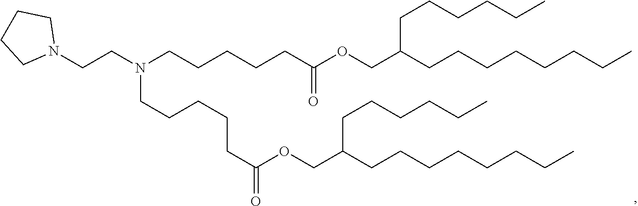 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof.

In some embodiments, the RNA composition (e.g., the mRNA composition or the circRNA composition) and methods for making and using thereof include a lipid having the compound structure:

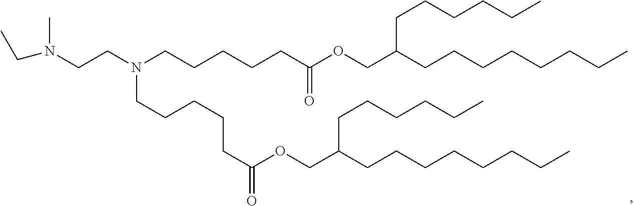 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof.

In some embodiments, the RNA composition (e.g., the mRNA composition or the circRNA composition) and methods for making and using thereof include a lipid having the compound structure:

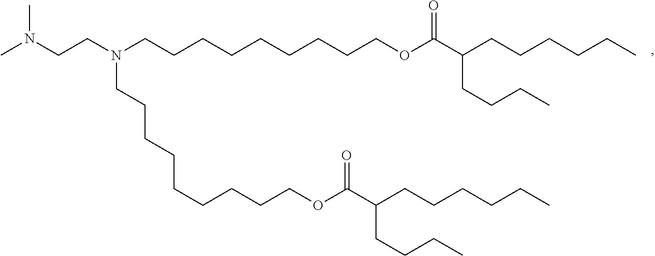 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable lipids for use in the RNA composition (e.g., the mRNA composition or the circRNA composition) and methods for making and using thereof include the lipids as described in International Patent Publication WO 2017/004143, which is incorporated herein by reference in its entirety.

In some embodiments, the RNA composition (e.g., the mRNA composition or the circRNA composition) and methods for making and using thereof include a lipid having the compound structure:

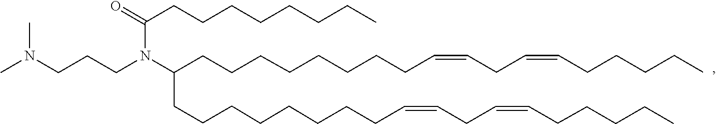 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof.

In some embodiments, the RNA composition (e.g., the mRNA composition or the circRNA composition) and methods for making and using thereof include a lipid having the compound structure:

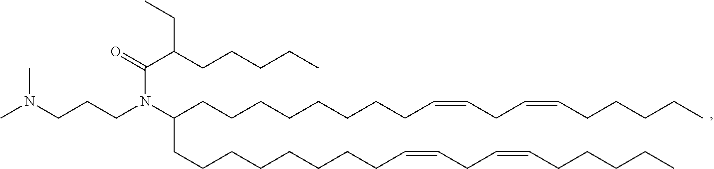 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof.

In some embodiments, the RNA composition (e.g., the mRNA composition or the circRNA composition) and methods for making and using thereof include a lipid having the compound structure:

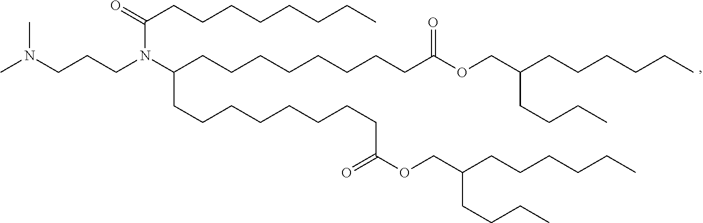 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof.

In some embodiments, the RNA composition (e.g., the mRNA composition or the circRNA composition) and methods for making and using thereof include a lipid having the compound structure:

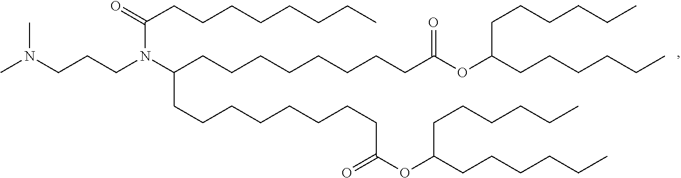 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the RNA composition (e.g., the mRNA composition or the circRNA composition) and methods for making and using thereof include a lipid having the compound structure:

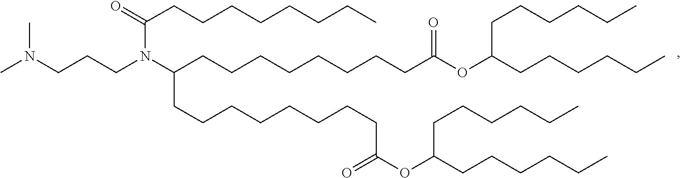 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the RNA composition (e.g., the mRNA composition or the circRNA composition) and methods for making and using thereof include a lipid having the compound structure:

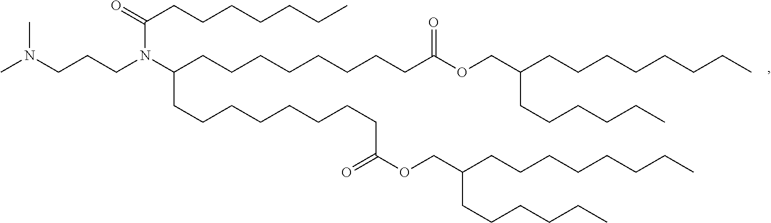 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof.

In some embodiments, the RNA composition (e.g., the mRNA composition or the circRNA composition) and methods for making and using thereof include a lipid having the compound structure:

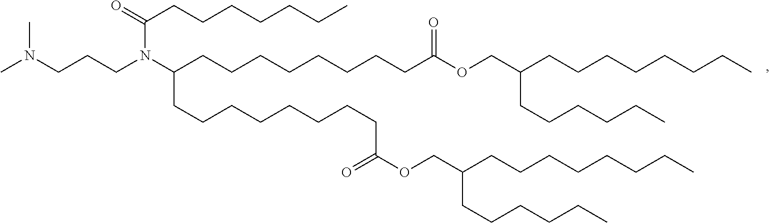 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof.

In some embodiments, the RNA composition (e.g., the mRNA composition or the circRNA composition) and methods for making and using thereof include a lipid having the compound structure:

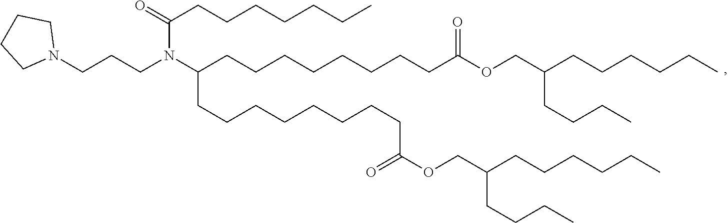 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the RNA composition (e.g., the mRNA composition or the circRNA composition) and methods for making and using thereof include a lipid having the compound structure:

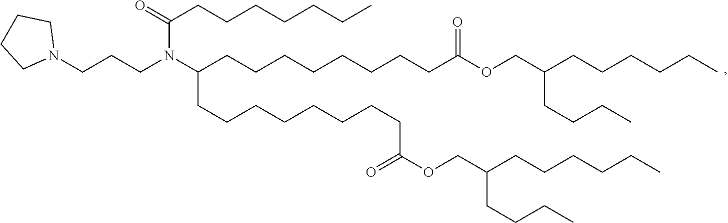 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the RNA composition (e.g., the mRNA composition or the circRNA composition) and methods for making and using thereof include a lipid having the compound structure:

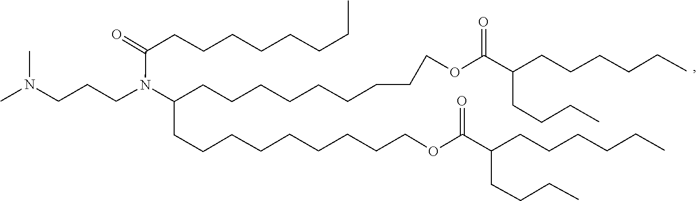 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof.

In some embodiments, the RNA composition (e.g., the mRNA composition or the circRNA composition) and methods for making and using thereof include a lipid having the compound structure:

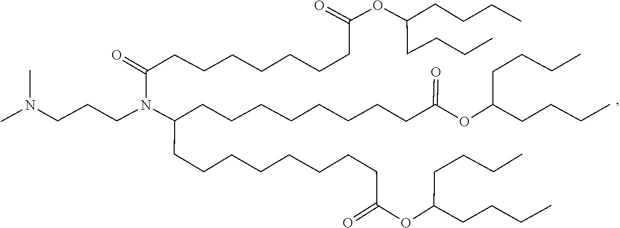 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof.

In some embodiments, the RNA composition (e.g., the mRNA composition or the circRNA composition) and methods for making and using thereof include a lipid having the compound structure:

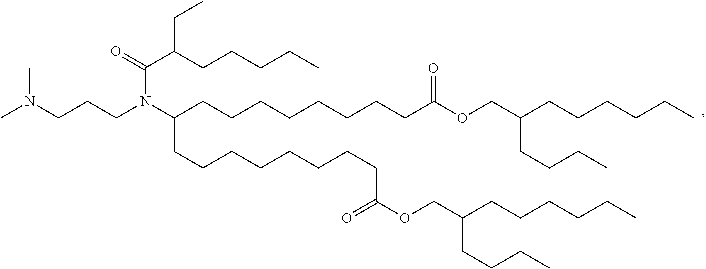 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof.

In some embodiments, the RNA composition (e.g., the mRNA composition or the circRNA composition) and methods for making and using thereof include a lipid having the compound structure:

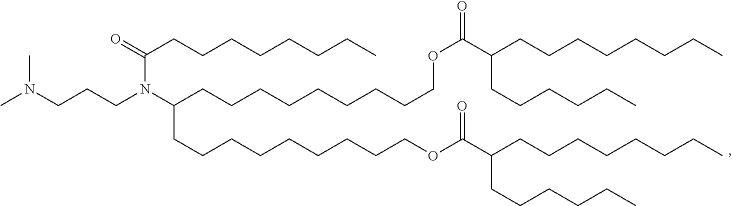 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof.

In some embodiments, the RNA composition (e.g., the mRNA composition or the circRNA composition) and methods for making and using thereof include a lipid having the compound structure:

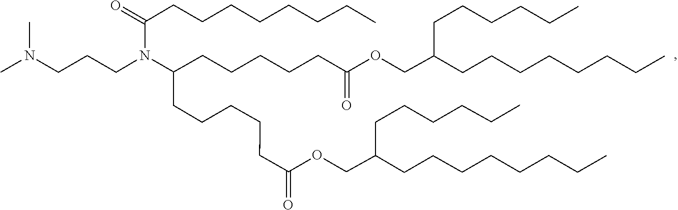 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof.

In some embodiments, the RNA composition (e.g., the mRNA composition or the circRNA composition) and methods for making and using thereof include a lipid having the compound structure:

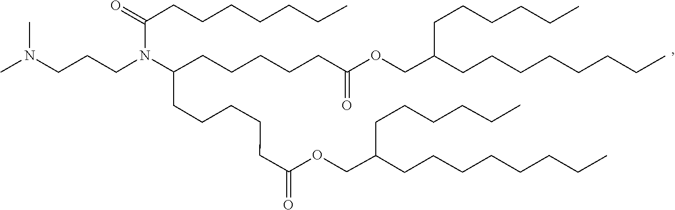 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof.

In some embodiments, the RNA composition (e.g., the mRNA composition or the circRNA composition) and methods for making and using thereof include a lipid having the compound structure:

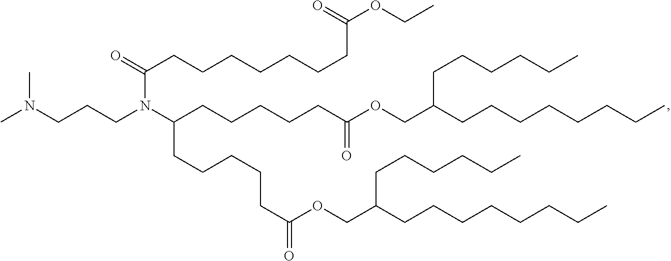 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof.

In some embodiments, the RNA composition (e.g., the mRNA composition or the circRNA composition) and methods for making and using thereof include a lipid having the compound structure:

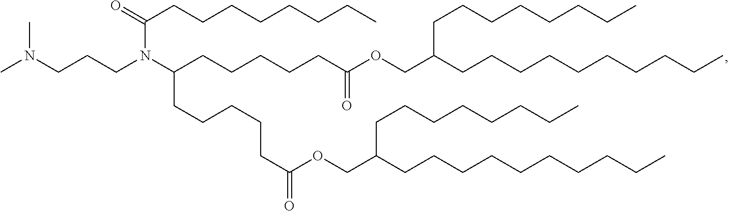 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable lipids for use in the RNA composition (e.g., the mRNA composition or the circRNA composition) and methods for making and using thereof include the lipids as described in International Patent Publication WO 2017/075531, which is incorporated herein by reference in its entirety.

In some embodiments, the RNA composition (e.g., the mRNA composition or the circRNA composition) and methods for making and using thereof include a lipid of the following formula:

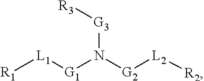

or a pharmaceutically acceptable salt thereof, wherein one of L¹ or L² is —O(C═O)—, —(C═O)O—, —C(═O)—, —O—, —S(O)x, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or
—NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable lipids for use in the RNA composition (e.g., the mRNA composition or the circRNA composition) and methods for making and using thereof include the lipids as described in International Patent Publication WO 2017/117528, which is incorporated herein by reference in its entirety. In some embodiments, the RNA composition (e.g., the mRNA composition or the circRNA composition) and methods for making and using thereof include a lipid having the compound structure:

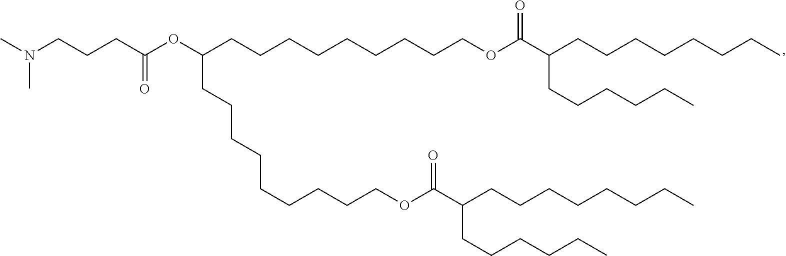 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof.

In some embodiments, the RNA composition (e.g., the mRNA composition or the circRNA composition) and methods for making and using thereof include a lipid having the compound structure:

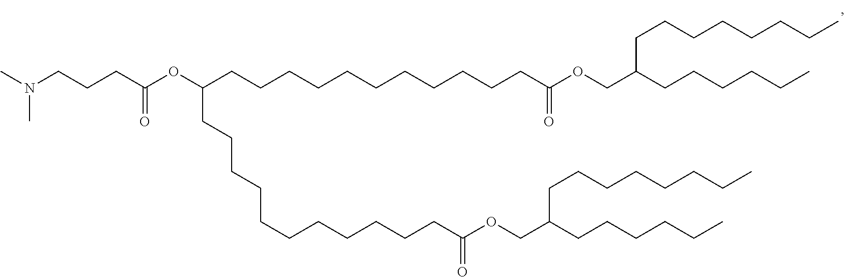 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof.

In some embodiments, the RNA composition (e.g., the mRNA composition or the circRNA composition) and methods for making and using thereof include a lipid having the compound structure:

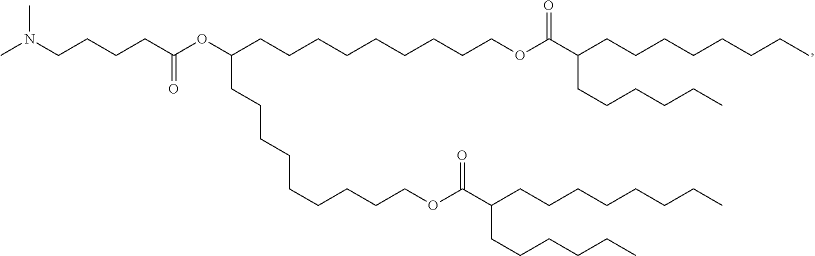 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable lipids for use in the RNA composition (e.g., the mRNA composition or the circRNA composition) and methods for making and using thereof include the lipids as described in International Patent Publication WO 2017/049245, which is incorporated herein by reference in its entirety.

In some embodiments, the lipids of the RNA composition (e.g., the mRNA composition or the circRNA composition) and methods for making and using thereof include a compound of the following formulas:

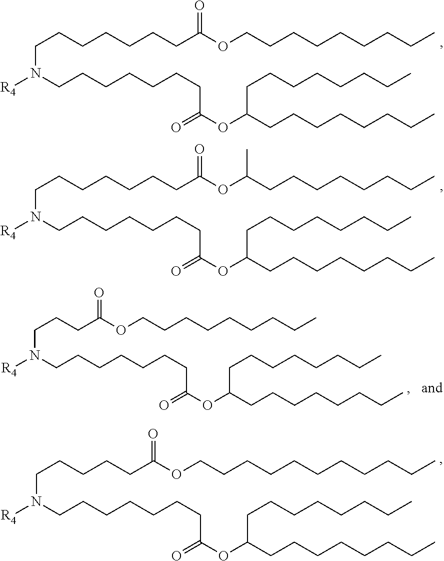

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3.

In certain embodiments, the RNA composition (e.g., the mRNA composition or the circRNA composition) and methods for making and using thereof include a lipid having a compound structure of:

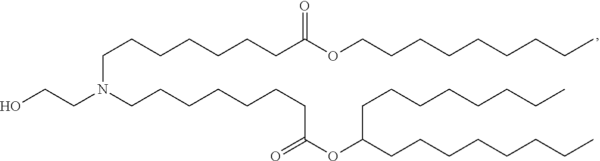 [SMILES: CCCCCCCCCOC(=O)CCCCCCCN(CCO)CCCCCCCC(=O)OC(CCCCCCCC)CCCCCCCC]

and pharmaceutically acceptable salts thereof.

In certain embodiments, the RNA composition (e.g., the mRNA composition or the circRNA composition) and methods for making and using thereof include a lipid having a compound structure of:

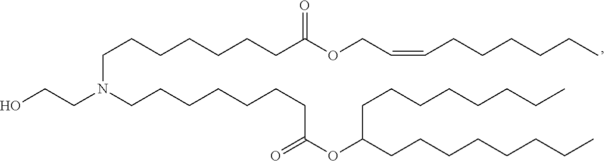 [SMILES: CCCCCC/C=C\COC(=O)CCCCCCCN(CCO)CCCCCCCC(=O)OC(CCCCCCCC)CCCCCCCC]

and pharmaceutically acceptable salts thereof.

In certain embodiments, the RNA composition (e.g., the mRNA composition or the circRNA composition) and methods for making and using thereof include a lipid having a compound structure of:

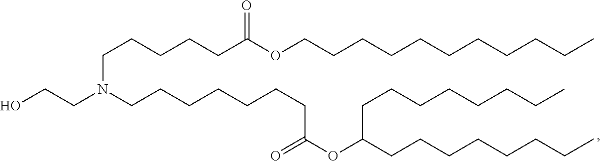 [SMILES: CCCCCCCCCCCOC(=O)CCCCCN(CCO)CCCCCCCC(=O)OC(CCCCCCCC)CCCCCCCC ; ","]

and pharmaceutically acceptable salts thereof.

In certain embodiments, the RNA composition (e.g., the mRNA composition or the circRNA composition) and methods for making and using thereof include a lipid having a compound structure of:

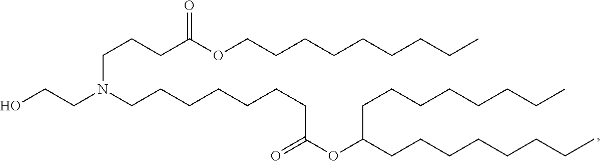 [SMILES: CCCCCCCCCOC(=O)CCCN(CCO)CCCCCCCC(=O)OC(CCCCCCCC)CCCCCCCC ; ","]

and pharmaceutically acceptable salts thereof.

Other suitable lipids for use in the RNA composition (e.g., the mRNA composition or the circRNA composition) and methods for making and using thereof include the lipids as described in International Patent Publication WO 2017/173054 and WO 2015/095340, each of which is incorporated herein by reference in its entirety. In certain embodiments, the RNA composition (e.g., the mRNA composition or the circRNA composition) and methods for making and using thereof include a lipid having a compound structure of:

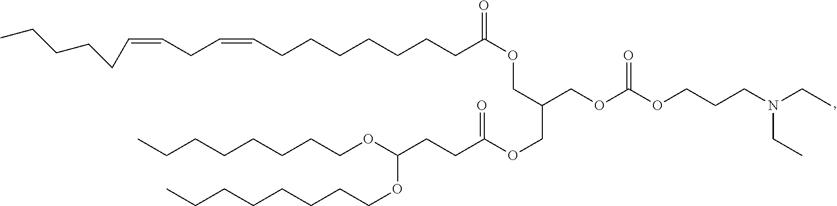 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof.

In certain embodiments, the RNA composition (e.g., the mRNA composition or the circRNA composition) and methods for making and using thereof include a lipid having a compound structure of:

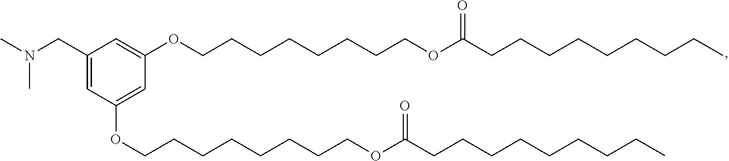 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof.

In certain embodiments, the RNA composition (e.g., the mRNA composition or the circRNA composition) and methods for making and using thereof include a lipid having a compound structure of:

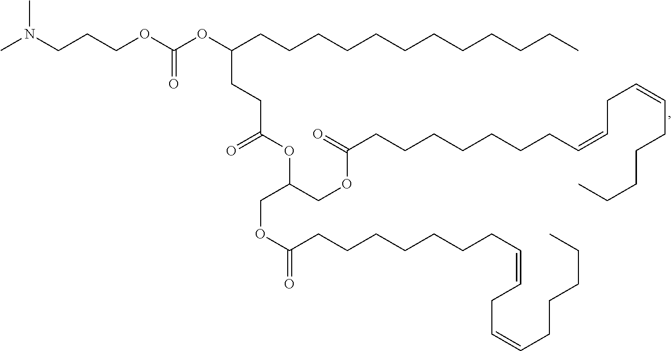 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof.

In certain embodiments, the RNA composition (e.g., the mRNA composition or the circRNA composition) and methods for making and using thereof include a lipid having a compound structure of:

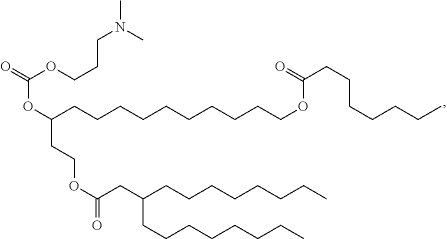 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

In some embodiments, the LNMPs described herein may include a ionizable lipid as described in, may be formulated as described in, or may comprise or be comprised by a composition as described in WO2016118724, WO2016118725, WO2016187531, WO2017176974, WO2018078053, WO2019027999, WO2019036030, WO2019089828, WO2019099501, WO2020072605, WO2020081938, WO2020118041, WO2020146805, or WO2020219876, each of which is incorporated by reference herein in its entirety.

The ionizable lipids disclosed herein may be used to form the LNMP composition together with one or more natural lipids disclosed herein. In some embodiments, the LNMP composition is formulated to further comprise one or more therapeutic agents. In some embodiments, the LNMP composition is a lipid nanoparticle that encapsulates or is associated with the one or more mRNA compositions.

In some embodiments, the RNA composition (e.g., mRNA composition or circRNA composition) disclosed herein has an N/P ratio of at least 3, for instance, an N/P ratio of 3 to 100, 3 to 50, 3 to 30, 3 to 20, 3 to 15, 3 to 12, 3 to 10, 6 to 30, 6 to 20, 6 to 15, or 6 to 12. For example, the N/P ratio may be 6±1, or the N/P ratio may be 6±0.5. In some embodiments, the N/P ratio is about 6. In some embodiments, the N/P ratio is about 3 (e.g., 3±1 or 3±0.5). In some embodiments, the RNA composition (e.g., mRNA composition or circRNA composition) has an N/P ratio of about 12 to about 17, for example, the N/P ratio is about 15±1, or the N/P ratio is about 15±0.5. In some embodiments, the N/P ratio is about 15. In some embodiments, the N/P ratio is about 12 (e.g., 12±1 or 12±0.5).

In some embodiments, the disclosure relates to a composition comprising (i) one or more compounds chosen from the ionizable lipids of Formula (I)—(III), pharmaceutically acceptable salts thereof, and stereoisomers of any of the foregoing and (ii) a lipid component. In some embodiments, the composition comprises 5%, 10%, 15%, 20%, 25%, 30%, 35%, 40%, 45%, 50%, 55%, 60%, 65%, 70%, 75%, 80%, 85%, 90%, or 95% of the one or more compounds.

In some embodiments, the disclosure relates to a composition comprising (i) one or more lipid nanoparticles and (ii) one or more lipid components.

In some embodiments, one or more lipid components comprise one or more helper lipids and one or more PEG lipids. In some embodiments, the lipid component(s) comprise(s) one or more helper lipids, one or more PEG lipids, and one or more neutral lipids.

Non-limiting examples of neutral lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids may be acyl groups derived from fatty acids having C₁₀-C₂₄ carbon chains, e.g., lauroyl, myristoyl, palmitoyl, stearoyl, or oleoyl.

In some embodiments, the RNA composition (e.g., mRNA composition or circRNA composition) comprises a phytosterol or a combination of a phytosterol and cholesterol. In some embodiments, the phytosterol is selected from the group consisting of b-sitosterol, stigmasterol, b-sitostanol, campesterol, brassicasterol, and combinations thereof. In some embodiments, the phytosterol is selected from the group consisting of b-sitosterol, b-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In some embodiments, the phytosterol is selected from the group consisting of Compound S-140, Compound S-151, Compound S-156, Compound S-157, Compound S-159, Compound S-160, Compound S-164, Compound S-165, Compound S-170, Compound S-173, Compound S-175, and combinations thereof. In some embodiments, the phytosterol is a combination of Compound S-141, Compound S-140, Compound S-143 and Compound S-148. In some embodiments, the phytosterol comprises a sitosterol or a salt or an ester thereof. In some embodiments, the phytosterol comprises a stigmasterol or a salt or an ester thereof.

### Other Lipids and Other Agents

The exogenous lipid may be a cell-penetrating agent, may be capable of increasing delivery of a polypeptide by the LNMP to a cell, and/or may be capable of increasing loading (e.g., loading efficiency or loading capacity) of a polypeptide. Further exemplary exogenous lipids include sterols and PEGylated lipids.

The LNMPs can be modified with other components (e.g., lipids, e.g., sterols, e.g., cholesterol; or small molecules) to further alter the functional and structural characteristics of the LNMP. For example, the LNMPs can be further modified with stabilizing molecules that increase the stability of the LNMPs (e.g., for at least one day at room temperature, and/or stable for at least one week at 4° C.).

In some embodiments, the LNMP is modified with a sterol, e.g., sitosterol, sitostanol, β-sitosterol, 7α-hydroxycholesterol, pregnenolone, cholesterol (e.g., ovine cholesterol or cholesterol isolated from plants), stigmasterol, campesterol, fucosterol, or an analog (e.g., a glycoside, ester, or peptide) of any sterol. In some examples, the exogenous sterol is added to the preparation prior to step (b), e.g., mixed with extracted NMP lipids prior to step (b). The exogenous sterol may be added to amount to, e.g., 1%, 5%, 10%, 20%, 30%, 40%, 50%, 60%, 70%, 80%, 90%, or more than 90% (w/w) of total lipids and sterols in the preparation.

In some embodiments, the sterol is cholesterol or sitosterol. In some instances, the LNMPs comprise a molar ratio of least 1%, 5%, 10%, 15%, 20%, 25%, 30%, 35%, 40%, 45%, 50%, 55%, 60%, or more than 60% sterol (e.g., cholesterol or sitosterol), e.g., 1%-10%, 10%-20%, 20%-30%, 30%-40%, 40%-50%, or 50%-60% sterol. In some embodiments, the LNMP comprises a molar ratio of about 35%-50% sterol (e.g., cholesterol or sitosterol), e.g., about 36%, 38.5%, 42.5%, or 46.5% sterol. In some embodiments, the LNMP comprises a molar ratio of about 20%-40% sterol.

In some embodiments, a LNMP that has been modified with a sterol has altered stability (e.g., increased stability) relative to a LNMP that has not been modified with a sterol. In some aspects, a LNMP that has been modified with a sterol has a greater rate of fusion with a membrane of a target cell relative to a LNMP that has not been modified with a sterol.

In some instances, the LNMPs comprise an exogenous lipid and an exogenous sterol.

In some embodiments, the LNMP is modified with a PEGylated lipid. Polyethylene glycol (PEG) length can vary from 1 kDa to 10 kDa; in some aspects, PEG having a length of 2 kDa is used. In some embodiments, the PEGylated lipid is C14-PEG2k, C18-PEG2k, or DMPE-PEG2k. In some instances, the LNMPs comprise a molar ratio of at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 10%, 20%, 30%, 40%, 50%, or more than 50% PEGylated lipid (e.g., C14-PEG2k, C18-PEG2k, or DMPE-PEG2k), e.g., 0.1%-0.5%, 0.5%-1%, 1%-1.5%, 1.5%-2.5%, 2.5%-3.5%, 3.5%-5%, 5%-10%, 10%-20%, 20%-30%, 30%-40%, or 30%-50% PEGylated lipid. In some embodiments, the LNMP comprises a molar ratio of about 0.1%-10% PEGylated lipid (e.g., C14-PEG2k, C18-PEG2k, or DMPE-PEG2k), e.g., about 1%-3% PEGylated lipid, e.g., about 1.5% or about 2.5% PEGylated lipid. In some embodiments, a LNMP that has been modified with a PEGylated lipid has altered stability (e.g., increased stability) relative to a LNMP that has not been modified with a PEGylated lipid. In some embodiments, a LNMP that has been modified with a PEGylated lipid has altered particle size relative to a LNMP that has not been modified with a PEGylated lipid. In some embodiments, a LNMP that has been modified with a PEGylated lipid is less likely to be phagocytosed than a LNMP that has not been modified with a PEGylated lipid. The addition of PEGylated lipids can also affect stability in GI tract and enhance particle migration through mucus. PEG may be used as a method to attach targeting moieties.

In some embodiments, the LNMPs are modified with an ionizable lipid (e.g., C12-200 or MC3) and one or both of a sterol (e.g., cholesterol or sitosterol) and a PEGylated lipid (e.g., C14-PEG2k, C18-PEG2k, or DMPE-PEG2k).

In some embodiments, the modified LNMPs comprise a molar ratio of about 5%-50% LNMP lipids (e.g., about 10%-20% LNMP lipids, e.g., about 10%, 12.5%, 16%, or 20% LNMP lipids); about 30%-75% ionizable lipids (e.g., about 35% or about 50% ionizable lipids); about 35%-50% sterol (e.g., about 36%, 38.5%, 42.5%, or 46.5% sterol); and about 0.1%-10% PEGylated lipid (e.g., about 1%-3% PEGylated lipid, e.g., about 1.5% or about 2.5% PEGylated lipid).

In some embodiments, the modified LNMPs comprise a molar ratio of about 5%-60% LNMP lipids (e.g., about 10%-20%, 20%-30%, 30%-40%, 40%-50%, or 50%-60% LNMP lipids, e.g., about 10%, 12.5%, 16%, 20%, 30%, 40%, 50%, or 60% LNMP lipids); about 25%-75% ionizable lipids (e.g., about 35% or about 50% ionizable lipids); about 10%-50% sterol (e.g., about 10%, 12.5%, 14%, 16%, 18%, 20%, 36%, 38.5%, 42.5%, or 46.5% sterol); and about 0.1%-10% PEGylated lipid (e.g., about 0.5%-5% PEGylated lipid, e.g., about 1%-3% PEGylated lipid, or about 1.5% or about 2.5% PEGylated lipid).

In some embodiments, the ionizable lipids, LNMP lipids, sterol, and PEGylated lipid comprise about 25%-75%, about 20%-60%, about 10%-45%, and about 0.5%-5%, respectively, of the lipids in the modified NMP.

In some embodiments, the ionizable lipids, natural lipids, sterol, and PEGylated lipid comprise about 30%-75%, about 20%-50%, about 10%-45%, and about 1%-5%, respectively, of the lipids in the modified NMP.

In some embodiments, the ionizable lipids, natural lipids, sterol, and PEGylated lipid comprise about 35%-75%, about 20%-50%, about 10%-45%, and about 1%-5%, respectively, of the lipids in the modified NMP.

In some embodiments, the ionizable lipids, natural lipids, sterol, and PEGylated lipid are formulated at a molar ratio of about 35:50:12.5:2.5.

In some embodiments, the ionizable lipids, natural lipids, sterol, and PEGylated lipid are formulated at a molar ratio of about 35:50:11.5:3.5.

In some embodiments, the ionizable lipids, natural lipids, sterol, and PEGylated lipid are formulated at a molar ratio of about 35:20:42.5:2.5.

In some embodiments, a LNMP has been modified with an ionizable lipid (and/or cationic lipid) and a sterol and/or a PEGylated lipid more efficiently encapsulates a negatively charged cargo (e.g., a nucleic acid) than a LNMP that has not been modified with an ionizable lipid (and/or cationic lipid) and a sterol and/or a PEGylated lipid. The modified LNMP may have an encapsulation efficiency for the cargo (e.g., nucleic acid, e.g., RNA or DNA) that is at least 5%, 10%, 15%, 20%, 30%, 40%, 50%, 60%, 70%, 80%, 90%, 95%, 98%, 99%, or more than 99%, e.g., may have an encapsulation efficiency of 5%-30%, 30%-50%, 50%-70%, 70%-80%, 80%-90%, 90%-95%, or 95%-100%.

Cell uptake of the modified LNMPs can be measured by a variety of methods known in the art. For example, the LNMPs, or a component thereof, can be labelled with a marker (e.g., a fluorescent marker) that can be detected in isolated cells to confirm uptake.

In some embodiments, a LNMP formulation provided herein comprises two or more different modified LNMPs, e.g., comprises modified LNMPs derived from different unmodified LNMPs (e.g., unmodified LNMPs from two or more different natural sources) and/or comprises modified LNMPs comprising different species and/or different ratios of ionizable lipids, sterols, and/or PEGylated lipids.

In some instances, the organic solvent in which the lipid film is dissolved is dimethylformamide:methanol (DMF:MeOH). Alternatively, the organic solvent or solvent combination may be, e.g., acetonitrile, acetone, ethanol, methanol, dimethylformamide, tetrahydrofuran, 1-buthanol, dimethyl sulfoxide, acetonitrile:ethanol, acetonitrile:methanol, acetone:methanol, methyl tert-butyl ether:propanol, tetrahydrofuran:methanol, dimethyl sulfoxide:methanol, or dimethylformamide:methanol.

The aqueous phase may be any suitable solution, e.g., a citrate buffer (e.g., a citrate buffer having a pH of about 3.2), water, or phosphate-buffered saline (PBS). The aqueous phase may further comprise a nucleic acid (e.g., an siRNA or siRNA precursor (e.g., dsRNA), miRNA or miRNA precursor, mRNA, circRNA, or plasmid (pDNA)) or a small molecule.

The lipid solution and the aqueous phase may be mixed in the microfluidics device at any suitable ratio. In some examples, aqueous phase and the lipid solution are mixed at a 3:1 volumetric ratio.

LNMPs may optionally include additional agents, e.g., cell-penetrating agents, therapeutic agents, polynucleotides, polypeptides, or small molecules. The LNMPs can carry or associate with additional agents in a variety of ways to enable delivery of the agent to a target plant or animal, e.g., by encapsulating the agent, incorporation of the agent in the lipid bilayer structure, or association of the agent (e.g., by conjugation) with the surface of the lipid bilayer structure. Nucleic acid molecules can be incorporated into the LNMPs either in vivo or in vitro (e.g., in tissue culture, in cell culture, or synthetically incorporated).

### Zeta Potential

The LNMPs comprising an ionizable lipid and optionally a cationic lipid (e.g., DC-cholesterol or DOTAP) may have, e.g., a zeta potential of greater than −30 mV when in the absence of cargo, greater than −20 mV, greater than −5 mV, greater than 0 mV, or about 30 my when in the absence of cargo. In some examples, the LNMP has a negative zeta potential, e.g., a zeta potential of less than 0 mV, less than −10 mV, less than −20 mV, less than −30 mV, less than −40 mV, or less than −50 mV when in the absence of cargo. In some examples, the LNMP has a positive zeta potential, e.g., a zeta potential of greater than 0 mV, greater than 10 mV, greater than 20 mV, greater than 30 mV, greater than 40 mV, or greater than 50 mV when in the absence of cargo. In some examples, the LNMP has a zeta potential of about 0.

The zeta potential of the LNMP may be measured using any method known in the art. Zeta potentials are generally measured indirectly, e.g., calculated using theoretical models from the data obtained using methods and techniques known in the art, e.g., electrophoretic mobility or dynamic electrophoretic mobility. Electrophoretic mobility is typically measured using microelectrophoresis, electrophoretic light scattering, or tunable resistive pulse sensing. Electrophoretic light scattering is based on dynamic light scattering. Typically, zeta potentials are accessible from dynamic light scattering (DLS) measurements, also known as photon correlation spectroscopy or quasi-elastic light scattering.

### Plant EV-Markers

The LNMPs in the RNA composition (e.g., mRNA composition or circRNA composition) and methods of making and using thereof may have a range of markers that identify the LNMPs (e.g. LPMPs) as being produced using a plant EV, and/or including a segment, portion, or extract thereof. As used herein, the term “plant EV-marker” refers to a component that is naturally associated with a plant and incorporated into or onto the plant EV in planta, such as a plant protein, a plant nucleic acid, a plant small molecule, a plant lipid, or a combination thereof. Examples of plant EV-markers can be found, for example, in Rutter and Innes, Plant Physiol. 173(1): 728-741, 2017; Raimondo et al., Oncotarget. 6(23): 19514, 2015; Ju et al., Mol. Therapy. 21(7):1345-1357, 2013; Wang et al., Molecular Therapy. 22(3): 522-534, 2014; and Regente et al, J of Exp. Biol. 68(20): 5485-5496, 2017; each of which is incorporated herein by reference.

Additional examples of the suitable plant EV-markers include those described and listed in International Patent Application Publication No. WO 2021/041301, which is incorporated herein by reference in its entirety.

### Bacterial EV-Markers

The bacterial components (e.g., bacterial lipids) in the bacteria-derived lipid composition and methods of making and using thereof may have a range of markers that identify the bacterial component as being produced. As used herein, the term “bacterial EV-marker” refers to a component that is naturally associated with a bacterium and incorporated into or onto the bacterial EV, such as a bacterial protein, a bacterial nucleic acid, a bacterial small molecule, a bacterial lipid, or a combination thereof.

### Natural Source EV-Markers

The NMPs may have a range of markers that identify the NMP as being produced from a specific source EV, and/or including a segment, portion, or extract thereof. As used herein, the term “EV-marker” refers to a component that is naturally associated with a specific source and incorporated into or onto the EV in vivo, such as a protein, a nucleic acid, a small molecule, a lipid, or a combination thereof. Examples of source EV-markers include but are not limited to peptidoglycan, lipopolysaccharide, ester-linked lipids, ether-linked lipids, circular DNA, chitin, beta-glucan, pekilo, mycoprotein, cerato-platanins, exotoxins, diacylglycerol, triglycerides, phosphatidylcholine, phosphatidylinositol, ornithine lipids, glycolipids, sphingolipids, hopanoids, or ergosterol.

The source EV marker can include a lipid. Examples of lipid markers that may be found in the NMP include lipid A, lipopolysaccharide, ergosterol, ornithine lipids (OLs), sulfolipids, diacylglyceryl-N,N,N-trimethylhomoserine (DGTS), glycolipids (GLs), diacylglycerol (DAG), hopanoids (HOPs), glucosyleramide, sterylglycosides, ether-linked lipids, or a combination thereof.

Other EV markers may include lipids that accumulate in sources in response to abiotic or biotic stressors.

Alternatively, the source EV marker may include a protein. In some instances, the protein EV marker may be an antimicrobial or antiviral protein naturally produced by the source, including proteins that are secreted in response to abiotic or biotic stressors. Some examples of protein EV markers include but are not limited to cecropins, moricins, defensins, proline- and glycine-rich peptides, fungal immunomodulatory proteins, flagellin, encapsulin, streptavidin, internalin, pilin, halocin, or archaeocins. In some instances, the EV marker can include a protein involved in lipid metabolism. In some instances, the protein EV marker is a cellular trafficking protein in the source. In certain instances where the EV marker is a protein, the protein marker may lack a signal peptide that is typically associated with secreted proteins. Unconventional secretory proteins seem to share several common features like (i) lack of a leader sequence, (ii) absence of PTMs specific for ER or Golgi apparatus, and/or (iii) secretion not affected by brefeldin A which blocks the classical ER/Golgi-dependent secretion pathway. One skilled in the art can use a variety of tools freely accessible to the public to evaluate a protein for a signal sequence, or lack thereof.

In instances where the EV marker is a protein, the protein may have an amino acid sequence having at least 35%, 40%, 45%, 50%, 55%, 60%, 65%, 70%, 75%, 80%, 85%, 90%, 95%, 98%, 99%, or 100% sequence identity to a known EV marker.

In some instances, the EV marker includes a nucleic acid encoded in the source, e.g., an Arthropod, Plant, Fungi, Archaea, or Bacteria RNA, DNA, or PNA. For example, the NMP may include dsRNA, mRNA, a circular RNA (circRNA), a viral RNA, a microRNA (miRNA), or a small interfering RNA (siRNA) encoded by the source. In some instances, the nucleic acid may be one that is associated with a protein that facilitates the long-distance transport of RNA. In some instances, the nucleic acid EV marker may be one involved in host-induced gene silencing (HIGS), which is the process by which a source silences foreign transcripts of pathogens. In some instances, the nucleic acid may be a microRNA.

In instances where the EV marker is a nucleic acid, the nucleic acid may have a nucleotide sequence having at least 35%, 40%, 45%, 50%, 55%, 60%, 65%, 70%, 75%, 80%, 85%, 90%, 95%, 98%, 99%, or 100% sequence identity to a known EV marker.

In some instances, the EV marker includes a compound produced by the source. For example, the compound may a component of the cell wall (e.g. lipopolysaccharide). For example, the compound may be a defense compound produced in response to abiotic or biotic stressors, such as pathogens or extreme environmental stress.

In some instances, the NMP may also be identified as being produced from a source EV based on the lack of certain markers (e.g., lipids, polypeptides, or polynucleotides) that are not typically produced by these sources, but are generally associated with other organisms (e.g., markers of animal EVs or plant EVs). For example, in some instances, the NMP lacks lipids typically found in animal EVs or plant EVs.

EV markers can be identified using any approaches known in the art that enable identification of small molecules (e.g., mass spectroscopy, mass spectrometry), lipids (e.g., mass spectroscopy, mass spectrometry), proteins (e.g., mass spectroscopy, immunoblotting), or nucleic acids (e.g., PCR analysis). In some instances, a NMP composition described herein includes a detectable amount, e.g., a pre-determined threshold amount, of an EV marker described herein.

### Loading of Agents (e.g., Nucleic Acids)

The LNMPs are modified to include a therapeutic agent (e.g., a nucleic acid molecule) to form the RNA composition (e.g., mRNA composition or circRNA composition). The LNMPs can carry or associate with such agents by a variety of means to enable delivery of the agent to a target organism (e.g., a target animal), e.g., by encapsulating the agent, incorporation of the component in the lipid bilayer structure, or association of the component (e.g., by conjugation) with the surface of the lipid bilayer structure of the LNMP. In some instances, the agent is included in the LNMP formulation, as described herein.

The agent can be incorporated or loaded into or onto the LNMPs by any methods known in the art that allow association, directly or indirectly, between the LNMPs and agent. The agents can be incorporated into the LNMPs by an in vivo method (e.g., in planta, e.g., through production of LNMPs from a transgenic plant that comprises the agent), or in vitro (e.g., in tissue culture, or in cell culture), or both in vivo and in vitro methods.

In some instances, the LNMPs are loaded in vitro. The substance may be loaded onto or into (e.g., may be encapsulated by) the LNMPs using, but not limited to, physical, chemical, and/or biological methods (e.g., in tissue culture or in cell culture). For example, the agent may be introduced into LNMPs by one or more of electroporation, sonication, passive diffusion, stirring, lipid extraction, or extrusion. In some instances, the agent is incorporated into the LNMP using a microfluidic device, e.g., using a method in which LNMP lipids are provided in an organic phase, the heterologous functional agent is provided in an aqueous phase, and the organic and aqueous phases are combined in the microfluidics device to produce a LNMP comprising the heterologous functional agent. Loaded LNMPs can be assessed to confirm the presence or level of the loaded agent using a variety of methods, such as HPLC (e.g., to assess small molecules), immunoblotting (e.g., to assess proteins); and/or quantitative PCR (e.g., to assess nucleotides). However, it should be appreciated by those skilled in the art that the loading of a substance of interest into LNMPs is not limited to the above-illustrated methods.

In some instances, the agent can be conjugated to the LNMP, in which the agent is connected or joined, indirectly or directly, to the LNMP. For instance, one or more agents can be chemically linked to a LNMP, such that the one or more agents are joined (e.g., by covalent or ionic bonds) directly to the lipid bilayer of the LNMP. In some instances, the conjugation of various agents to the LNMPs can be achieved by first mixing the one or more agents with an appropriate cross-linking agent (e.g., N-ethylcarbo-diimide (“EDC”), which is generally utilized as a carboxyl activating agent for amide bonding with primary amines and also reacts with phosphate groups) in a suitable solvent. After a period of incubation sufficient to allow the agent to attach to the cross-linking agent, the cross-linking agent/agent mixture can then be combined with the LNMPs and, after another period of incubation, subjected to a sucrose gradient (e.g., and 8, 30, 45, and 60% sucrose gradient) to separate the free agent and free LNMPs from the agent conjugated to the LNMPs. As part of combining the mixture with a sucrose gradient, and an accompanying centrifugation step, the LNMPs conjugated to the agent are then seen as a band in the sucrose gradient, such that the conjugated LNMPs can then be collected, washed, and dissolved in a suitable solution for use as described herein.

In some instances, the LNMPs are stably associated with the agent prior to and following delivery of the LNMP, e.g., to an animal. In other instances, the LNMPs are associated with the agent such that the agent becomes dissociated from the LNMPs following delivery of the LNMP, e.g., to an animal.

The LNMPs can be loaded or the LNMP can be formulated with various concentrations of the agent, depending on the particular agent or use. For example, in some instances, the LNMPs are loaded or the LNMP is formulated such that the LNMP formulation disclosed herein includes about 0.001, 0.01, 0.1, 1.0, 2, 3, 4, 5, 6, 7, 8, 9, 10, 15, 20, 30, 40, 50, 60, 70, 80, 90, or 95 (or any range between about 0.001 and 95) or more wt % of an agent. In some instances, the LNMPs are loaded or the LNMP is formulated such that the LNMP formulation includes about 95, 90, 80, 70, 60, 50, 40, 30, 20, 15, 10, 9, 8, 7, 6, 5, 4, 3, 2, 1.0, 0.1, 0.01, 0.001 (or any range between about 95 and 0.001) or less wt % of an agent. For example, the LNMP formulation can include about 0.001 to about 0.01 wt %, about 0.01 to about 0.1 wt %, about 0.1 to about 1 wt %, about 1 to about 5 wt %, or about 5 to about 10 wt %, about 10 to about 20 wt % of the agent. In some instances, the LNMP can be loaded or the LNMP is formulated with about 1, 5, 10, 50, 100, 200, or 500, 1,000, 2,000 (or any range between about 1 and 2,000) or more μg/ml of an agent. A LNMP of the invention can be loaded or a LNMP can be formulated with about 2,000, 1,000, 500, 200, 100, 50, 10, 5, 1 (or any range between about 2,000 and 1) or less μg/ml of an agent.

In some instances, the LNMPs are loaded or the LNMP is formulated such that the LNMP formulation disclosed herein includes at least 0.001 wt %, at least 0.01 wt %, at least 0.1 wt %, at least 1.0 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 30 wt %, at least 40 wt %, at least 50 wt %, at least 60 wt %, at least 70 wt %, at least 80 wt %, at least 90 wt %, or at least 95 wt % of an agent. In some instances, the LNMP can be loaded or the LNMP can be formulated with at least 1 μg/ml, at least 5 μg/ml, at least 10 μg/ml, at least 50 μg/ml, at least 100 μg/ml, at least 200 μg/ml, at least 500 μg/ml, at least 1,000 μg/ml, at least 2,000 μg/ml of an agent.

In some instances, the LNMP is formulated with the agent by suspending the LNMPs in a solution comprising or consisting of the agent, e.g., suspending or resuspending the LNMPs by vigorous mixing. The agent (e.g., cell-penetrating agent, e.g., nucleic acids, enzyme, detergent, ionic, fluorous, or zwitterionic liquid, or ionizable lipid may comprise, e.g., less than 1% or at least 1%, 5%, 10%, 20%, 30%, 40%, 50%, 60%, 70%, 80%, 90%, or 100% of the solution.

### Pharmaceutical Formulations

The modified LNMPs are formulated into pharmaceutical compositions (i.e., an RNA composition such as a mRNA composition or a circRNA composition), e.g., for administration to an animal (e.g., a human). The pharmaceutical composition may be administered to an animal (e.g., human) with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed.

The LNMP/RNA composition (e.g., mRNA composition or circRNA composition) may be formulated for e.g., oral administration, intravenous administration (e.g., injection or infusion), intramuscular, or subcutaneous administration to an animal. For injectable formulations, various effective pharmaceutical carriers are known in the art (See, e.g., Remington: The Science and Practice of Pharmacy, 22^{nd} ed., (2012) and ASHP Handbook on Injectable Drugs, 18^{th} ed., (2014)).

Suitable pharmaceutically acceptable carriers and excipients are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The LNMP/RNA composition (e.g., mRNA composition or circRNA composition) may be formulated according to conventional pharmaceutical practice. The concentration of the compound in the formulation will vary depending upon a number of factors, including the dosage of the active agent (e.g., LNMPs and nucleic acids) to be administered, and the route of administration.

For oral administration to an animal, the LNMP/RNA composition (e.g., mRNA composition or circRNA composition) can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium). The compositions disclosed herein may also further include an immediate-release, extended release or delayed-release formulation.

For parenteral administration to an animal, the LNMP/RNA composition (e.g., mRNA composition or circRNA composition) may be formulated in the form of liquid solutions or suspensions and administered by a parenteral route (e.g., subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), and F-12 medium). Formulation methods are known in the art, see e.g., Gibson (ed.) Pharmaceutical Preformulation and Formulation (2nd ed.) Taylor & Francis Group, CRC Press (2009).

### Polynucleotides

The LNMP/RNA composition (e.g., mRNA composition or circRNA composition) includes one or more nucleic acid molecules, e.g., polynucleotides, which encode one or more wild type or engineered proteins, peptides, or polypeptides. Exemplary polynucleotides, e.g., polynucleotide constructs, include antigen-encoding RNA polynucleotides, e.g., mRNAs, linear polyribonucleotides, or circRNAs.

Examples of polypeptides that can be used herein can include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

Polypeptides included herein may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

The polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

The LNMP/RNA composition (e.g., mRNA composition or circRNA composition) may include any number or type (e.g., classes) of polypeptides, such as at least about any one of 1 polypeptide, 2, 3, 4, 5, 10, 15, 20, or more polypeptides. A suitable concentration of each polypeptide in the LNMP/mRNA composition depends on factors such as efficacy, stability of the polypeptide, number of distinct polypeptides in the formulation, and methods of application of the formulation. In some instances, each polypeptide in a liquid formulation is from about 0.1 ng/mL to about 100 mg/mL. In some instances, each polypeptide in a solid formulation is from about 0.1 ng/g to about 100 mg/g.

### Nucleic Acids Encoding Peptides

In some instances, the LNMP/RNA composition (e.g., mRNA composition or circRNA composition) include a heterologous nucleic acid encoding a polypeptide. Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range therebetween.

The LNMP/RNA composition (e.g., mRNA composition or circRNA composition) may also include active variants of a nucleic acid sequence of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the invention includes an active polypeptide encoded by a nucleic acid variant as described herein. In some instances, the active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Certain methods for expressing a nucleic acid encoding a protein may involve expression in cells, including insect, yeast, plant, bacteria, or other cells under the control of appropriate promoters. Expression vectors may include nontranscribed elements, such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instance, provided is a composition to alter expression of one or more proteins, e.g., proteins that affect activity, structure, or function. Expression of the one or more proteins may be restricted to a specific location(s) or widespread throughout the organism.

mRNA

The LNMP/mRNA composition may include a mRNA molecule, e.g., a mRNA molecule encoding a polypeptide. The mRNA molecule can be synthetic and modified (e.g., chemically). The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some instances, the modified RNA agent of interest described herein has modified nucleosides or nucleotides. Such modifications are known and are described, e.g., in WO 2012/019168. Additional modifications are described, e.g., in WO 2015/038892; WO 2015/038892; WO 2015/089511; WO 2015/196130; WO 2015/196118 and WO 2015/196128 A2, which are herein incorporated by reference in their entirety.

In some instances, the modified RNA encoding a polypeptide of interest has one or more terminal modification, e.g., a 5′ cap structure and/or a poly-A tail (e.g., of between 100-200 nucleotides in length). The 5′ cap structure may be selected from the group consisting of CapO, CapI, ARCA, inosine, NI-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5′ UTR including at least one Kozak sequence, and a 3′ UTR. Such modifications are known and are described, e.g., in WO 2012/135805 and WO 2013/052523, which are incorporated herein by reference in their entirety. Additional terminal modifications are described, e.g., in WO 2014/164253 and WO 2016/011306, WO 2012/045075, and WO 2014/093924, which are incorporated herein by reference in their entirety. Chimeric enzymes for synthesizing capped RNA molecules (e.g., modified mRNA) which may include at least one chemical modification are described in WO 2014/028429, which is incorporated herein by reference in its entirety.

In some instances, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667 and WO 2013/151736, which are incorporated herein by reference in their entirety. Methods of purification include purifying an RNA transcript including a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified mRNA sample to DNAse treatment (WO 2014/152030).

Formulations of modified RNAs are known and are described, e.g., in WO 2013/090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; Tables 6, 185 and 186 of International Publication No WO 2013/151667; which are incorporated herein by reference in their entirety. Any of the foregoing may be synthesized as an IVT polynucleotide, chimeric polynucleotide or a circular polynucleotide, and each may include one or more modified nucleotides or terminal modifications.

### Inhibitory RNA

In some instances, the LNMP/RNA composition (e.g., mRNA composition or circRNA composition) includes an inhibitory RNA molecule, e.g., that acts via the RNA interference (RNAi) pathway. In some instances, the inhibitory RNA molecule decreases the level of gene expression in a target organism and/or decreases the level of a protein in the target organism. In some instances, the inhibitory RNA molecule inhibits expression of a gene. For example, an inhibitory RNA molecule may include a short interfering RNA or its precursor, short hairpin RNA, and/or a microRNA or its precursor that targets a gene in the target organism. Certain RNA molecules can inhibit gene expression through the biological process of RNA interference (RNAi). RNAi molecules include RNA or RNA-like structures typically containing 15-50 base pairs (such as about 18-25 base pairs) and having a nucleobase sequence identical (or complementary) or nearly identical (or substantially complementary) to a coding sequence in an expressed target gene within the cell. RNAi molecules include, but are not limited to short interfering RNAs (siRNAs), double-strand RNAs (dsRNA), short hairpin RNAs (shRNA), meroduplexes, dicer substrates, and multivalent RNA interference (U.S. Pat. Nos. 8,084,599 8,349,809, 8,513,207 and 9,200,276, which are incorporated herein by reference in their entirety). The inhibitory RNA molecule can be chemically synthesized or transcribed in vitro.

Additional examples of the inhibitory RNA molecules include those described in details in International Patent Application Publication No. WO 2021/041301, which is incorporated herein by reference in its entirety.

### Circular Polyribonucleotides

The LNMP/circRNA therapeutic composition includes a circular polyribonucleotide molecule, e.g., a circRNA encoding a polypeptide. The circular polyribonucleotide comprises the elements as described below as well as an expression sequence encoding the polypeptide. In some embodiments, the circular polyribonucleotide includes any feature, or any combination of features as disclosed in International Patent Publication No. WO2019/118919, which is hereby incorporated by reference in its entirety.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide is between 500 nucleotides and 20,000 nucleotides, between 1,000 and 20,000 nucleotides, between 2,000 and 20,000 nucleotides, or between 5,000 and 20,000 nucleotides. In some embodiments, the circular polyribonucleotide is between 500 nucleotides and 10,000 nucleotides, between 1,000 and 10,000 nucleotides, between 2,000 and 10,000 nucleotides, or between 5,000 and 10,000 nucleotides.

### Internal Ribosome Entry Sites

In some embodiments, a circular polyribonucleotide described herein includes one or more internal ribosome entry site (IRES) elements. In some embodiments, the IRES is operably linked to one or more expression sequences (e.g., each IRES is operably linked to one or more expression sequences, where each expression sequence optionally encodes a polypeptide). In embodiments, the IRES is located between a heterologous promoter and the 5′ end of a coding sequence (e.g., a coding sequence encoding a polypeptide)

A suitable IRES element to include in a polyribonucleotide (e.g., the polyribonucleotide cargo of the polyribonucleotide) includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is from the DNA of an organism including, but not limited to, a virus, a mammal, and a Drosophila. Such viral DNA may be from, but is not limited to, picomavirus complementary DNA (cDNA), with encephalomyocarditis virus (EMCV) cDNA and poliovirus cDNA. In one embodiment, Drosophila DNA from which an IRES element is from includes, but is not limited to, an Antennapedia gene from Drosophila melanogaster.

In some embodiments, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2 (HRV-2), Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus (AEV), Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-I, Human BCL2, Human BiP, Human c-IAPI, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-I, Simian picomavirus, Turnip crinkle virus, Aichivirus, Crohivirus, Echovirus 11, an aptamer to elF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus. In a further embodiment, the IRES is an IRES sequence of Theiler's encephalomyelitis virus.

The IRES sequence may have a modified sequence in comparison to the wild-type IRES sequence. In some embodiments, when the last nucleotide of the wild-type IRES is not a cytosine nucleic acid residue, the last nucleotide of the wild-type IRES sequence may be modified such that it is a cytosine residue. For example, the IRES sequence may be a CVB3 IRES sequence wherein the terminal adenosine residue is modified to cytosine residue. In some embodiments, the modified CVB3 IRES may have the nucleic acid sequence of:

(SEQ ID NO: 14)
TTAAAACAGCCTGTGGGTTGATCCCACCCACAGGCCCATTGGGCGCTAGCA
CTCTGGTATCACGGTACCTTTGTGCGCCTGTTTTATACCCCCTCCCCCAAC
TGTAACTTAGAAGTAACACACACCGATCAACAGTCAGCGTGGCACACCAGC
CACGTTTTGATCAAGCACTTCTGTTACCCCGGACTGAGTATCAATAGACTG
CTCACGCGGTTGAAGGAGAAAGCGTTCGTTATCCGGCCAACTACTTCGAAA
AACCTAGTAACACCGTGGAAGTTGCAGAGTGTTTCGCTCAGCACTACCCCA
GTGTAGATCAGGTCGATGAGTCACCGCATTCCCCACGGGCGACCGTGGCGG
TGGCTGCGTTGGCGGCCTGCCCATGGGGAAACCCATGGGACGCTCTAATAC
AGACATGGTGCGAAGAGTCTATTGAGCTAGTTGGTAGTCCTCCGGCCCCTG
AATGCGGCTAATCCTAACTGCGGAGCACACACCCTCAAGCCAGAGGGCAGT
GTGTCGTAACGGGCAACTCTGCAGCGGAACCGACTACTTTGGGTGTCCGTG
TTTCATTTTATTCCTATACTGGCTGCTTATGGTGACAATTGAGAGATCGTT
ACCATATAGCTATTGGATTGGCCATCCGGTGACTAATAGAGCTATTATATA
TCCCTTTGTTGGGTTTATACCACTTAGCTTGAAAGAGGTTAAAACATTACA
ATTCATTGTTAAGTTGAATACAGCAAC

In some embodiments, the IRES sequence is an Enterovirus 71 (EV17) IRES. In some embodiments, the terminal guanosine residue of the EV17 IRES sequence is modified to a cytosine residue. In some embodiments, the modified EV71 IRES may have the nucleic acid sequence of:

(SEQ ID NO: 15)
TTAAAACAGCTGTGGGTTGTCACCCACCCACAGGGTCCACTGGGCGCTAGT
ACACTGGTATCTCGGTACCTTTGTACGCCTGTTTTATACCCCCTCCCTGAT
TTGCAACTTAGAAGCAACGCAAACCAGATCAATAGTAGGTGTGACATACCA
GTCGCATCTTGATCAAGCACTTCTGTATCCCCGGACCGAGTATCAATAGAC
TGTGCACACGGTTGAAGGAGAAAACGTCCGTTACCCGGCTAACTACTTCGA
GAAGCCTAGTAACGCCATTGAAGTTGCAGAGTGTTTCGCTCAGCACTCCCC
CCGTGTAGATCAGGTCGATGAGTCACCGCATTCCCCACGGGCGACCGTGGC
GGTGGCTGCGTTGGCGGCCTGCCTATGGGGTAACCCATAGGACGCTCTAAT
ACGGACATGGCGTGAAGAGTCTATTGAGCTAGTTAGTAGTCCTCCGGCCCC
TGAATGCGGCTAATCCTAACTGCGGAGCACATACCCTTAATCCAAAGGGCA
GTGTGTCGTAACGGGCAACTCTGCAGCGGAACCGACTACTTTGGGTGTCCG
TGTTTCTTTTTATTCTTGTATTGGCTGCTTATGGTGACAATTAAAGAATTG
TTACCATATAGCTATTGGATTGGCCATCCAGTGTCAAACAGAGCTATTGTA
TATCTCTTTGTTGGATTCACACCTCTCACTCTTGAAACGTTACACACCCTC
AATTACATTATACTGCTGAACACGAAGCGGCCACC

In some embodiments, the polyribonucleotide (e.g., the polyribonucleotide cargo of the polyribonucleotide) includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide (e.g., the polyribonucleotide cargo of the polyribonucleotide) includes one or more IRES sequences on one or both sides of each expression sequence, leading to separation of the resulting peptide(s) and or polypeptide(s). For example, a polyribonucleotide (e.g., the polyribonucleotide cargo of the polyribonucleotide) described herein may include a first IRES operably linked to a first expression sequence (e.g., encoding a first polypeptide) and a second IRES operably linked to a second expression sequence (e.g., encoding a second polypeptide).

In some embodiments, a polyribonucleotide (e.g., the polyribonucleotide cargo of the polyribonucleotide) described herein includes an IRES (e.g., an IRES operably linked to a coding region). For example, the polyribonucleotide (e.g., the polyribonucleotide cargo of the polyribonucleotide) may include any IRES as described in Chen et al. Mol. Cell 81(20):4300-4318, 2021; Jopling et al. Oncogene 20:2664-2670, 2001; Baranick et al. PNAS 105(12):4733-4738, 2008; Lang et al. Molecular Biology of the Cell 13(5):1792-1801, 2002; Dorokhov et al. PNAS 99(8):5301-5306, 2002; Wang et al. Nucleic Acids Research 33(7):2248-2258, 2005; Petz et al. Nucleic Acids Research 35(8):2473-2482, 2007, Chen et al. SCIENCE 268:415-417, 1995; Fan et al. NATURE COMMUNICATION 13(1):3751-3765, 2022, and International Publication No. WO2021/263124 each of which is hereby incorporated by reference in their entirety.

### Regulatory Elements

In some embodiments, the circular polyribonucleotide includes a regulatory element, e.g., a sequence that modifies expression of an expression sequence within the circular polyribonucleotide. In some embodiments, the polyribonucleotide described herein (e.g., the polyribonucleotide cargo of the polyribonucleotide) includes one or more regulatory elements.

A regulatory element may include a sequence that is located adjacent to an expression sequence that encodes an expression product (e.g., polypeptide). A regulatory element may be operatively linked to the adjacent sequence. A regulatory element may increase an amount of product expressed as compared to an amount of the expressed product when no regulatory element exists. A regulatory element may be used to increase the expression of one or more polypeptide(s) encoded by a polyribonucleotide (e.g., the polyribonucleotide cargo of the polyribonucleotide). Likewise, a regulatory element may be used to decrease the expression of one or more polypeptide(s) encoded by a polyribonucleotide (e.g., the polyribonucleotide cargo of the polyribonucleotide). In some embodiments, a regulatory element may be used to increase expression of a polypeptide and another regulatory element may be used to decrease expression of another polypeptide on the same polyribonucleotide (e.g., the polyribonucleotide cargo of the polyribonucleotide). In addition, one regulatory element can increase amount(s) of product(s) (e.g., polypeptide(s)) expressed for multiple expression sequences attached in tandem. Hence, one regulatory element can enhance the expression of one or more expression sequences. Multiple regulatory elements can also be used, for example, to differentially regulate expression of different expression sequences.

In some embodiments, the regulatory element is a translation modulator. A translation modulator can modulate translation of the expression sequence in the circular polyribonucleotide. A translation modulator can be a translation enhancer or suppressor. In some embodiments, the circular polyribonucleotide includes at least one translation modulator adjacent to at least one expression sequence. In some embodiments, the circular polyribonucleotide includes a translation modulator adjacent each expression sequence. In some embodiments, the translation modulator is present on one or both sides of each expression sequence, leading to separation of the expression products, e.g., polypeptide(s).

In some embodiments, the regulatory element is a microRNA (miRNA) or a miRNA binding site.

Further examples of regulatory elements are described, e.g., in paragraphs [0154]-[0161] of International Patent Publication No. WO2019/118919, which is hereby incorporated by reference in its entirety.

### Signal Sequences

In some embodiments, a polypeptide expressed from a circular or linear polyribonucleotide disclosed herein includes a secreted protein, for example, a protein that naturally includes a signal sequence, or one that does not usually encode a signal sequence but is modified to contain one. In some embodiments, the polypeptide encoded by the circular or linear polyribonucleotide includes a secretion signal. For example, the secretion signal may be the naturally encoded secretion signal for a secreted protein. In another example, the secretion signal may be a modified secretion signal for a secreted protein. In other embodiments, the polypeptide encoded by the circular or linear polyribonucleotide does not include a secretion signal.

In some embodiments, a polyribonucleotide (e.g., circular polyribonucleotide, linear polyribonucleotide, polyribonucleotide cargo of the polyribonucleotide) encodes multiple copies of the same polypeptide (e.g., one, two, three, four, five, six, seven, eight, nine, ten, or more). In some embodiments, at least one copy of the polypeptide includes a signal sequence and at least one copy of the polypeptide does not include a signal sequence. In some embodiments, a polyribonucleotide (e.g., circular polyribonucleotide, linear polyribonucleotide, polyribonucleotide cargo of the polyribonucleotide) encodes plurality of polypeptides (e.g., a plurality of different polypeptides or a plurality of polypeptides having less than 100% sequence identity), where at least one of the plurality of polypeptides includes a signal sequence and at least one copy of the plurality of polypeptides does not include a signal sequence.

In some embodiments, the signal sequence is a wild-type signal sequence that is present on the N-terminus of the corresponding wild-type polypeptide, e.g., when expressed endogenously. In some embodiments, the signal sequence is heterologous to the polypeptide, e.g., is not present when the wild-type polypeptide is expressed endogenously. A polyribonucleotide sequence encoding a polypeptide may be modified to remove the nucleotide sequence encoding a wild-type signal sequence and/or add a sequence encoding a heterologous signal sequence.

A polypeptide encoded by a polyribonucleotide may include a signal sequence that directs the polypeptide to the secretory pathway. In some embodiments, the signal sequence may direct the polypeptide to reside in certain organelles (e.g., the endoplasmic reticulum, Golgi apparatus, or endosomes). In some embodiments, the signal sequence directs the polypeptide to be secreted from the cell. For secreted proteins, the signal sequence may be cleaved after secretion, resulting in a mature protein. In other embodiments, the signal sequence may become embedded in the membrane of the cell or certain organelles, creating a transmembrane segment that anchors the protein to the membrane of the cell, endoplasmic reticulum, or Golgi apparatus. In certain embodiments, the signal sequence of a transmembrane protein is a short sequence at the N-terminal of the polypeptide. In other embodiments, the first transmembrane domain acts as the first signal sequence, which targets the protein to the membrane. In some embodiments, a polypeptide encoded by a polyribonucleotide includes either a secretion signal sequence, a transmembrane insertion signal sequence, or does not include a signal sequence.

### Cleavage Domains

A circular polyribonucleotide of the disclosure can include a cleavage domain (e.g., a stagger element or a cleavage sequence).

The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence—D(V/I)ExNPGP, where x=any amino acid (SEQ ID NO: 16). In some embodiments, the stagger element may include a chemical moiety, such as glycerol, a non-nucleic acid linking moiety, a chemical modification, a modified nucleic acid, or any combination thereof.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide includes one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

In some embodiments, the circular polyribonucleotide includes a stagger element. To avoid production of a continuous expression product, e.g., peptide or polypeptide, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular polyribonucleotide. The stagger element may include, but is not limited to a 2A-like, or CHYSEL (SEQ ID NO: 24) (cis-acting hydrolase element) sequence. In some embodiments, the stagger element encodes a sequence with a C-terminal consensus sequence that is X1X2X3EX5NPGP (SEQ ID NO: 38), where X1 is absent or G or H, X2 is absent or D or G, X3 is D or V or I or S or M, and X5 is any amino acid. In some embodiments, this sequence includes a non-conserved sequence of amino-acids with a strong alpha-helical propensity followed by the consensus sequence -D(V/I)EXNPGP (SEQ ID NO: 39), where x=any amino acid. Some nonlimiting examples of stagger elements includes GDVESNPGP (SEQ ID NO: 40), GDIEENPGP (SEQ ID NO: 41), VEPNPGP (SEQ ID NO: 42), IETNPGP (SEQ ID NO: 43), GDIESNPGP (SEQ ID NO: 44), GDVELNPGP (SEQ ID NO: 45), GDIETNPGP (SEQ ID NO: 46), GDVENPGP (SEQ ID NO: 47), GDVEENPGP (SEQ ID NO: 48), GDVEQNPGP (SEQ ID NO: 49), IESNPGP (SEQ ID NO: 50), GDIELNPGP (SEQ ID NO: 51), HDIETNPGP (SEQ ID NO: 52), HDVETNPGP (SEQ ID NO: 53), HDVEMNPGP (SEQ ID NO: 54), GDMESNPGP (SEQ ID NO: 55), GDVETNPGP (SEQ ID NO: 56), GDIEQNPGP (SEQ ID NO: 57), and DSEFNPGP (SEQ ID NO: 58).

In some embodiments, the stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element is present on one or both sides of each expression sequence, leading to translation of individual peptide(s) and or polypeptide(s) from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, the stagger element is present in the circular polyribonucleotide in other forms. For example, in some exemplary circular polyribonucleotides, a stagger element includes a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence.

In some embodiments, the first stagger element includes a termination element and separates an expression product of the first expression sequence from an expression product of its succeeding expression sequences, thereby creating discrete expression products. In some cases, the circular polyribonucleotide including the first stagger element upstream of the first translation initiation sequence of the succeeding sequence in the circular polyribonucleotide is continuously translated, while a corresponding circular polyribonucleotide including a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element includes a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first translation initiation sequence enables continuous translation of the first expression sequence and any succeeding expression sequences.

In some embodiments, the first stagger element separates one round expression product of the first expression sequence from the next round expression product of the first expression sequences, thereby creating discrete expression products. In some cases, the circular polyribonucleotide including the first stagger element upstream of the first translation initiation sequence of the first expression sequence in the circular polyribonucleotide is continuously translated, while a corresponding circular polyribonucleotide including a stagger element upstream of a second translation initiation sequence of a second expression sequence in the corresponding circular polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular polyribonucleotide than a distance between the first stagger element and the first translation initiation in the circular polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular polyribonucleotide includes more than one expression sequence.

Examples of stagger elements are described in paragraphs [0172]-[0175] of International Patent Publication No. WO2019/118919, which is hereby incorporated by reference in its entirety.

In some embodiments, a plurality of polypeptides encoded by a circular ribonucleotide may be separated by an IRES between each polypeptide (e.g., each polypeptide is operably linked to a separate IRES). For example, a circular polyribonucleotide may include a first IRES operably linked to a first expression sequence and a second IRES operably linked to a second expression sequence. The IRES may be the same IRES between all polypeptides. The IRES may be different between different polypeptides.

In some embodiments, the plurality of polypeptides may be separated by a 2A self-cleaving peptide. For example, a circular polyribonucleotide may encode an IRES operably linked to an open reading frame encoding a first polypeptide, a 2A, and a second polypeptide.

In some embodiments, the plurality of polypeptides may be separated by a protease cleavage site (e.g., a furin cleavage site). For example, a circular polyribonucleotide may encode an IRES operably linked to an open reading frame encoding a first polypeptide, a protease cleavage site (e.g., a furin cleavage site), and a second polypeptide.

In some embodiments, the plurality of polypeptides may be separated by a 2A self-cleaving peptide and a protease cleavage site (e.g., a furin cleavage site). For example, a circular polyribonucleotide may encode an IRES operably linked to an open reading frame encoding a first polypeptide, a 2A, a protease cleavage site (e.g., a furin cleavage site), and a second polypeptide. A circular polyribonucleotide may also encode an IRES operably linked to an open reading frame encoding a first polypeptide, a protease cleavage site (e.g., a furin cleavage site), a 2A, and a second polypeptide. A tandem 2A and furin cleavage site may be referred to as a furin-2A (which includes furin-2A or 2A-furin, arranged in either orientation).

Furthermore, the plurality of polypeptides encoded by the circular ribonucleotide may be separated by both IRES and 2A sequences. For example, an IRES may be between one polypeptide and a second polypeptide while a 2A peptide may be between the second polypeptide and the third polypeptide. The selection of a particular IRES or 2A self-cleaving peptide may be used to control the expression level of a polypeptide under control of the IRES or 2A sequence. For example, depending on the IRES and or 2A peptide selected, expression on the polypeptide may be higher or lower.

In some embodiments, a circular polyribonucleotide includes at least one cleavage sequence. In some embodiments, the cleavage sequence is adjacent to an expression sequence. In some embodiments, the cleavage sequence is between two expression sequences. In some embodiments, cleavage sequence is included in an expression sequence. In some embodiments, the circular polyribonucleotide includes from 2 to 10 cleavage sequences. In some embodiments, the circular polyribonucleotide includes from 2 to 5 cleavage sequences. In some embodiments, the multiple cleavage sequences are between multiple expression sequences; for example, a circular polyribonucleotide may include three expression sequences two cleavage sequences such that there is a cleavage sequence in between each expression sequence. In some embodiments, the circular polyribonucleotide includes a cleavage sequence, such as in an immolating circRNA or cleavable circRNA or self-cleaving circRNA. In some embodiments, the circular polyribonucleotide includes two or more cleavage sequences, leading to separation of the circular polyribonucleotide into multiple products, e.g., miRNAs, linear RNAs, smaller circular polyribonucleotide, etc.

In some embodiments, a cleavage sequence includes a ribozyme RNA sequence. A ribozyme (from ribonucleic acid enzyme, also called RNA enzyme or catalytic RNA) is an RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

In some embodiments, the cleavage sequence encodes a cleavable polypeptide linker. For example, a circular polyribonucleotide may encode two or more polypeptides, e.g., where the two or more polypeptides are encoded by a single open-reading frame (ORF). For example, two or more polypeptides may be encoded by a single open-reading frame, the expression of which is controlled by an IRES. In some embodiments, the ORF further encodes a polypeptide linker, e.g., such that the expression product of the ORF encodes two or more polypeptides each separated by a sequence encoding a polypeptide linker (e.g., a linker of 5-200, 5 to 100, 5 to 50, 5 to 20, 50 to 100, or 50 to 200 amino acids). The polypeptide linker may include a cleavage site, for example, a cleavage site recognized and cleaved by a protease (e.g., an endogenous protease in a subject following administration of the circular polyribonucleotide to that subject). In such embodiments, a single expression product including the amino acid sequence of two or more polypeptides is cleaved upon expression, such that the two or more polypeptides are separated following expression. Exemplary protease cleavage sites are known to those of skill in the art, for example, amino acid sequences that act as protease cleavage sites recognized by a metalloproteinase (e.g., a matrix metalloproteinase (MMP), such as any one or more of MMPs 1-28), a disintegrin and metalloproteinase (ADAM, such as any one or more of ADAMs 2, 7-12, 15, 17-23, 28-30 and 33), a serine protease (e.g., furin), urokinase-type plasminogen activator, matriptase, a cysteine protease, an aspartic protease, or a cathepsin protease. In some embodiments, the protease is MMP9 or MMP2. In some embodiments, the protease is matriptase.

In some embodiments, a circular polyribonucleotide described herein is an immolating circular polyribonucleotide, a cleavable circular polyribonucleotide, or a self-cleaving circular polyribonucleotide. A circular polyribonucleotide can deliver cellular components including, for example, RNA, long non-coding RNA (lncRNA), long intergenic non-coding RNA (lincRNA), microRNA (miRNA), transfer RNA (tRNA), ribosomal RNA (rRNA), small nucleolar RNA (snoRNA), non-coding RNA (ncRNA), small interfering RNA (siRNA), or small hairpin RNA (shRNA). In some embodiments, a circular polyribonucleotide includes miRNA separated by (i) self-cleavable elements; (ii) cleavage recruitment sites; (iii) degradable linkers; (iv) chemical linkers; and/or (v) spacer sequences. In some embodiments, circRNA includes siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and glmS ribozymes.

### Translation Initiation Sequences

In some embodiments, the circular polyribonucleotide described herein includes at least one translation initiation sequence. In some embodiments, the polyribonucleotide (e.g., the polyribonucleotide cargo of the polyribonucleotide) includes a translation initiation sequence operably linked to an expression sequence.

In some embodiments, the polyribonucleotide encodes a polypeptide and may include a translation initiation sequence, e.g., a start codon. In some embodiments, the translation initiation sequence includes a Kozak or Shine-Dalgarno sequence. In some embodiments, the polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the polyribonucleotide includes at least one translation initiation sequence adjacent to an expression sequence. In some embodiments, the translation initiation sequence provides conformational flexibility to the polyribonucleotide. In some embodiments, the translation initiation sequence is within a substantially single stranded region of the polyribonucleotide. Further examples of translation initiation sequences are described in paragraphs [0163]-[0165] of International Patent Publication No. WO2019/118919, which is hereby incorporated by reference in its entirety.

The polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, the polyribonucleotide may initiate at a codon which is not the first start codon, e.g., AUG. Translation of the polyribonucleotide may initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the polyribonucleotide may begin at alternative translation initiation sequence, such as ACG. As another non-limiting example, the polyribonucleotide translation may begin at alternative translation initiation sequence, CTG/CUG. As another non-limiting example, the polyribonucleotide translation may begin at alternative translation initiation sequence, GTG/GUG. As another non-limiting example, the polyribonucleotide may begin translation at a repeat-associated non-AUG (RAN) sequence, such as an alternative translation initiation sequence that includes short stretches of repetitive RNA e.g., CGG, GGGGCC (SEQ DI NO: 93), CAG, CTG.

### Termination Elements

In some embodiments, the polyribonucleotide described herein (e.g., the polyribonucleotide cargo of the polyribonucleotide) includes least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

Further examples of termination elements are described in paragraphs [0169]-[0170] of International Patent Publication No. WO2019/118919, which is hereby incorporated by reference in its entirety.

### Spacer Sequences

In some embodiments, the polyribonucleotide (e.g., circular polyribonucleotide, linear polyribonucleotide) described herein includes one or more spacer sequences. A spacer or spacer sequence refers to any contiguous nucleotide sequence (e.g., of one or more nucleotides) that provides distance or flexibility between two adjacent polynucleotide regions. Spacers may be present in between any of the nucleic acid elements described herein. Spacer may also be present within a nucleic acid element as described herein.

For example, wherein a nucleic acid includes any two or more of the following elements: (A) a 3′ catalytic intron fragment; (B) a 3′ splice site; (C) a 3′ exon fragment; (D) a polyribonucleotide cargo; (E) a 5′ exon fragment; (F) a 5′ splice site; and (G) a 5′ catalytic intron fragment; a spacer region may be present between any one or more of the elements. Any of elements (A), (B), (C), (D), (E), (F), or (G) may be separated by a spacer sequence, as described herein. For example, there may be a spacer between (A) and (B), between (B) and (C), between (C) and (D), between (D) and (E), between (E) and (F), or between (F) and (G).

In some embodiments, the polyribonucleotide (e.g., circular polyribonucleotide, linear polyribonucleotide) further includes a first spacer sequence between the 5′ exon fragment of (C) and the polyribonucleotide cargo of (D). The spacer may be, e.g., at least 5 (e.g., at least 10, at least 15, at least 20) ribonucleotides in length. In some embodiments, the polyribonucleotide further includes a second spacer sequence between the polyribonucleotide cargo of (D) and the 5′ exon fragment of (E).

A spacer sequence may be used to separate an IRES from adjacent structural elements to maintain the structure and function of the IRES or the adjacent element. A spacer can be specifically engineered depending on the IRES. In some embodiments, an RNA folding computer software, such as RNAFold, can be utilized to guide designs of the various elements of the vector, including the spacers. Thus, in one embodiment, the spacer sequence is between the IRES and the 3′ exon fragment or the 5′ exon fragment. In other embodiments, the spacer sequence is between the expression sequence and the 3′ exon fragment. In other embodiments, the spacer sequence is adjacent to the 5′ exon fragment or the 3′ exon fragment.

The spacer may be, e.g., at least 3 (e.g., at least 10, at least 15, at least 20) ribonucleotides in length. In some embodiments, each spacer sequence is at least 3 (e.g., at least 10, at least 15, at least 20) ribonucleotides in length. Each spacer region may be, e.g., from 5 to 800 (e.g., 10, 20, 30, 40, 50, 60, 70, 80, 90, 100, 150, 200, 250, 300, 350, 400, 450, 500, 550, 600, 650, 700, 750 or 800) ribonucleotides in length. In some embodiments, the spacer sequence is at least about 60 ribonucleotides in length. In some embodiments, the spacer sequence is from about 60 to about 650 ribonucleotides in length.

In some embodiments, the first spacer sequence, the second spacer sequence, or the first spacer sequence and the second spacer sequence may include a poly(X) sequence. In some embodiments the first spacer sequence and the second spacer sequence, or the first spacer sequence and the second spacer sequence, may include a poly(A) sequence. The first spacer sequence, the second spacer sequence, or the first spacer sequence and the second spacer sequence, may include a poly(A-C) sequence. In some embodiments, the first spacer sequence, the second spacer sequence, or the first spacer sequence and the second spacer sequence includes a poly(A-G) sequence. In some embodiments, the first spacer sequence, the second spacer sequence, or the first spacer sequence and the second spacer sequence includes a poly(A-T) sequence. In some embodiments, the first spacer sequence, the second spacer sequence, or the first spacer sequence and the second spacer sequence includes a random sequence.

Spacers may also be present within a nucleic acid region described herein. For example, a polynucleotide cargo region may include one or multiple spacers. Spacers may separate regions within the polynucleotide cargo.

In some embodiments, the spacer sequence can be, for example, at least 10 nucleotides in length, at least 15 nucleotides in length, or at least 70 nucleotides in length. In some embodiments, the spacer sequence is at least 4, 5, 6, 7, 8, 9, 10, 11, 12, 13, 14, 15, 16, 17, 18, 19, 20, 25, 30, 35, 40, 45, 50, 55, 60, 65, or 70 nucleotides in length. In some embodiments, the spacer sequence is no more than 800, 700, 600, 500, 400, 300, 300, 100, 90, 80, 70, 60, 50, 45, 40, 35 or 30 nucleotides in length. In some embodiments the spacer sequence is from 20 to 70 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length.

The spacer sequences may be poly(X) sequences, poly(A) sequences, poly(A-T) sequences, poly(A-U) sequences, poly(A-C) sequences, poly(A-G) sequences, poly(G), poly(C) sequences, poly(U) sequences, or random sequences.

Exemplary spacer sequences are described in paragraphs [0293]-[0302] of International Patent Publication No. WO2019/118919, which is hereby incorporated by reference in its entirety.

In some embodiments, the circular polyribonucleotide includes a 5′ spacer sequence (e.g., between the 5′ annealing region and the polyribonucleotide cargo). In some embodiments, the 5′ spacer sequence is at least about 60 nucleotides in length. In another embodiment, the 5′ spacer sequence is at least 100 nucleotides in length. In a further embodiment, the 5′ spacer sequence is at least about 200 nucleotides in length. In other embodiment, the 5′ spacer sequence is at least 300 nucleotides in length. In another embodiment, the 5′ spacer sequence is at least about 400 nucleotides in length. In another embodiment, the 5′ spacer sequence is at least about 500 nucleotides in length. In other embodiment, the 5′ spacer sequence is at least about 600 nucleotides in length. In other embodiment, the 5′ spacer sequence is at least 700 nucleotides in length. In one embodiment, the 5′ space is a poly(X) sequence. In one embodiment, the 5′ spacer sequence is a poly(A) sequence. In another embodiment, the 5′ spacer sequence is a poly(A-C) sequence. In some embodiments, the 5′ spacer sequence includes a poly(A-G) sequence. In some embodiments, the 5′ spacer sequence includes a poly(A-U) sequence. In some embodiments, the 5′ spacer sequence includes a random sequence.

In some embodiments, the polyribonucleotide (e.g., circular polyribonucleotide, linear polyribonucleotide) includes a 3′ spacer sequence (e.g., between the 3′ annealing region and the polyribonucleotide cargo). In some embodiments, the polyribonucleotide includes a 3′ spacer sequence (e.g., between the 3′ annealing region and the polyribonucleotide cargo). In some embodiments, the 3′ spacer sequence is at least about 60 nucleotides in length. In another embodiment, the 3′ spacer sequence is at least 100 nucleotides in length. In a further embodiment, the 3′ spacer sequence is at least about 200 nucleotides in length. In other embodiment, the 3′ spacer sequence is at least 300 nucleotides in length. In another embodiment, the 3′ spacer sequence is at least about 400 nucleotides in length. In another embodiment, the 3′ spacer sequence is at least about 500 nucleotides in length. In other embodiment, the 3′ spacer sequence is at least about 600 nucleotides in length. In other embodiment, the 3′ spacer sequence is at least 700 nucleotides in length. In one embodiment, the 3′ spacer sequence is a poly(X) sequence. In one embodiment, the 3′ spacer sequence is a poly(A) sequence. In another embodiment, the 3′ spacer sequence is a poly(A-C) sequence. In some embodiments, the 3′ spacer sequence includes a poly(A-G) sequence. In some embodiments, the 3′ spacer sequence includes a poly(A-U) sequence. In some embodiments, the 3′ spacer sequence includes a random sequence.

In one embodiment, the polyribonucleotide (e.g., circular polyribonucleotide, linear polyribonucleotide) includes a 5′ spacer sequence, but not a 3′ spacer sequence. In another embodiment, the circular polyribonucleotide includes a 3′ spacer sequence, but not a 5′ spacer sequence. In another embodiment, the polyribonucleotide includes neither a 5′ spacer sequence, nor a 3′ spacer sequence. In another embodiment, the polyribonucleotide does not include an IRES sequence. In a further embodiment, the polyribonucleotide does not include an IRES sequence, a 5′ spacer sequence or a 3′ spacer sequence.

In some embodiments, the spacer sequence includes at least 3 ribonucleotides, at least 4 ribonucleotides, at least 5 ribonucleotides, at least about 8 ribonucleotides, at least about 10 ribonucleotides, at least about 12 ribonucleotides, at least about 15 ribonucleotides, at least about 20 ribonucleotides, at least about 25 ribonucleotides, at least about 30 ribonucleotides, at least about 40 ribonucleotides, at least about 50 ribonucleotides, at least about 60 ribonucleotides, at least about 70 ribonucleotides, at least about 80 ribonucleotides, at least about 90 ribonucleotides, at least about 100 ribonucleotides, at least about 120 ribonucleotides, at least about 150 ribonucleotides, at least about 200 ribonucleotides, at least about 250 ribonucleotides, at least about 300 ribonucleotides, at least about 400 ribonucleotides, at least about 500 ribonucleotides, at least about 600 ribonucleotides, at least about 700 ribonucleotides, at least about 800 ribonucleotides, at least about 900 ribonucleotides, or at least about 1000 ribonucleotides.

In some embodiments, a circular polyribonucleotide includes a spacer sequence. In some embodiments, a linear polyribonucleotide includes a spacer sequence. In some embodiments, a spacer sequence may be included upstream of the translation initiation sequence of an expression sequence. In some embodiments, a spacer sequence may be included downstream of an expression sequence. In some instances, one spacer sequence for a first expression sequence is the same as or continuous with or overlapping with another spacer sequence for a second expression sequence. In some embodiments, the intron is a human intron. In some embodiments, the intron is a full-length human intron, e.g., ZKSCAN1.

Exemplary spacer sequences are described in paragraphs [0197]-[201] of International Patent Publication No. WO2019/118919, which is hereby incorporated by reference in its entirety.

In some embodiments, a circular polyribonucleotide includes a poly(A) sequence. In some embodiments, a linear polyribonucleotide includes a poly(A) sequence. Exemplary poly(A) sequences are described in paragraphs [0202]-[0205] of International Patent Publication No. WO2019/118919, which is hereby incorporated by reference in its entirety. In some embodiments, a circular polyribonucleotide lacks a poly(A) sequence. In some embodiments, a linear polyribonucleotide lacks a poly(A) sequence.

In some embodiments, a polyribonucleotide (e.g., circular polyribonucleotide, linear polyribonucleotide) includes a spacer sequence with one or more stretches of Adenosines and Uridines embedded within. These AU rich signatures may increase turnover rates of the expression product.

Introduction, removal, or modification of the spacer sequence AU rich elements (AREs) may be useful to modulate the stability, or immunogenicity (e.g., the level of one or more marker of an immune or inflammatory response) of a circular polyribonucleotide. When engineering specific polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any spacer sequence from any gene may be incorporated into the respective flanking regions of a circular polyribonucleotide.

In some embodiments, a circular polyribonucleotide lacks a 5′ spacer sequence and is competent for protein expression from its one or more expression sequences. In some embodiments, the circular polyribonucleotide lacks a 3′ spacer sequence and is competent for protein expression from its one or more expression sequences. In some embodiments, the circular polyribonucleotide lacks a poly(A) sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′ spacer sequence, a 3′ spacer sequence, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular polyribonucleotide lacks a 5′ spacer sequence. In some embodiments, the circular polyribonucleotide lacks a spacer sequence. In some embodiments, the circular polyribonucleotide lacks a poly(X) sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Production of Circular Polyribonucleotides

Circular polyribonucleotides may be prepared according to any available technique, including, but not limited to, recombinant technology and chemical synthesis. For example, a DNA molecule used to produce a circular RNA molecule can include a DNA sequence of a naturally occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification or mutagenesis of selected regions of a nucleic acid sequence, synthesis of oligonucleotide mixtures and ligation of mixture groups to “build” a mixture of nucleic acid molecules and combinations thereof.

In some embodiments, a linear polyribonucleotide for circularization may be cyclized, or concatemerized. In some embodiments, the linear polyribonucleotide for circularization may be cyclized in vitro prior to formulation and/or delivery. In some embodiments, the circular polyribonucleotide may be in a mixture with linear polyribonucleotides. In some embodiments, the linear polyribonucleotides have the same nucleic acid sequence as the circular polyribonucleotides.

In some embodiments, a linear polyribonucleotide for circularization is cyclized, or concatemerized using a chemical method to form a circular polyribonucleotide. In some chemical methods, the 5′-end and the 3-end of the nucleic acid (e.g., a linear polyribonucleotide for circularization) includes chemically reactive groups that, when close together, may form a new covalent linkage between the 5′-end and the 3-end of the molecule. The 5′-end may contain an NHS-ester reactive group and the 3-end may contain a 3-amino-terminated nucleotide such that in an organic solvent the 3-amino-terminated nucleotide on the 3-end of a linear RNA molecule will undergo a nucleophilic attack on the 5′-NHS-ester moiety forming a new 5′-/3′-amide bond. Other chemical methods of circularization include, but are not limited to click chemistry (e.g., alkyne and azide-based methods, or clickable bases), olefin metathesis, phosphoramidate ligation, hemiaminal-imine crosslinking, base modification, and any combination thereof.

In some embodiments, a linear primary construct or linear polyribonucleotide may be cyclized or concatenated to create a circular polyribonucleotide through methods such as, e.g., chemical, enzymatic, splint ligation, or ribozyme-catalyzed methods. The newly formed 5′-3′ linkage may be an intramolecular linkage or an intermolecular linkage. For example, a splint ligase, such as a SplintR® ligase, RNA ligase II, T4 RNA ligase, or T4 DNA ligase, can be used for splint ligation. According to this method, a single stranded polynucleotide (splint), such as a single-stranded DNA or RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular RNA. In some embodiments, a DNA or RNA ligase may be used in the synthesis of the circular polynucleotides. As a non-limiting example, the ligase may be a circ ligase or circular ligase.

In another example, either the 5′ or 3′ end of the linear polyribonucleotide can encode a ligase ribozyme sequence such that during in vitro transcription, the resultant linear circRNA includes an active ribozyme sequence capable of ligating the 5′ end of the linear polyribonucleotide to the 3′ end of the linear polyribonucleotide. The ligase ribozyme may be derived from the Group I Intron, Hepatitis Delta Virus, Hairpin ribozyme or may be selected by SELEX (systematic evolution of ligands by exponential enrichment).

In another example, a linear polyribonucleotide may be cyclized or concatenated by using at least one non-nucleic acid moiety. For example, the at least one non-nucleic acid moiety may react with regions or features near the 5′ terminus or near the 3′ terminus of the linear polyribonucleotide in order to cyclize or concatenate the linear polyribonucleotide. In another example, the at least one non-nucleic acid moiety may be located in or linked to or near the 5′ terminus or the 3′ terminus of the linear polyribonucleotide. The non-nucleic acid moieties may be homologous or heterologous. As a non-limiting example, the non-nucleic acid moiety may be a linkage such as a hydrophobic linkage, ionic linkage, a biodegradable linkage or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In another example, linear polyribonucleotides may be cyclized or concatenated by self-splicing. In some embodiments, the linear polyribonucleotides may include loop E sequence to self-ligate. In another embodiment, the linear polyribonucleotides may include a self-circularizing intron, e.g., a 5′ and 3′ slice junction, or a self-circularizing catalytic intron such as a Group I, Group II or Group III Introns. Nonlimiting examples of group I intron self-splicing sequences may include self-splicing permuted intron-exon sequences derived from T4 bacteriophage gene td, and the intervening sequence (IVS) rRNA of Tetrahymena, cyanobacterium Anabaena pre-tRNA-Leu gene, or a Tetrahymena pre-rRNA.

In some embodiments, the polyribonucleotide includes catalytic intron fragments, such as a 3′ half of Group I catalytic intron fragment and a 5′ half of Group I catalytic intron fragment. The first and second annealing regions may be positioned within the catalytic intron fragments. Group I catalytic introns are self-splicing ribozymes that catalyze their own excision from mRNA, tRNA, and rRNA precursors via two-metal ion phorphoryl transfer mechanism. Importantly, the RNA itself self-catalyzes the intron removal without the requirement of an exogenous enzyme, such as a ligase.

In some embodiments, the 3′ half of Group I catalytic intron fragment and the 5′ half of Group I catalytic intron fragment are from a cyanobacterium Anabaena pre-tRNA-Leu gene, or a Tetrahymena pre-rRNA.

In some embodiments, the 3′ half of Group I catalytic intron fragment and the 5′ half of Group I catalytic intron fragment are from a Cyanobacterium Anabaena pre-tRNA-Leu gene, and the 3′ exon fragment includes the first annealing region and the 5′ exon fragment includes the second annealing region. The first annealing region may include, e.g., from 5 to 50, e.g., from 10 to 15 (e.g., 10, 11, 12, 13, 14, or 15) ribonucleotides and the second annealing region may include, e.g., from 5 to 50, e.g., from 10 to 15 (e.g., 10, 11, 12, 13, 14, or 15) ribonucleotides.

In some embodiments, the 3′ half of Group I catalytic intron fragment and the 5′ half of Group I catalytic intron fragment are from a Tetrahymena pre-rRNA, and the 3′ half of Group I catalytic intron fragment includes the first annealing region and the 5′ exon fragment includes the second annealing region. In some embodiments, the 3′ exon includes the first annealing region and the 5′ half of Group I catalytic intron fragment includes the second annealing region. The first annealing region may include, e.g., from 6 to 50, e.g., from 10 to 16 (e.g., 10, 11, 12, 13, 14, 15, or 16) ribonucleotides, and the second annealing region may include, e.g., from 6 to 50, e.g., from 10 to 16 (e.g., 10, 11, 12, 13, 14, 15, or 16) ribonucleotides.

In some embodiments, the 3′ half of Group I catalytic intron fragment and the 5′ half of Group I catalytic intron fragment are from a cyanobacterium Anabaena pre-tRNA-Leu gene, a Tetrahymena pre-rRNA, or a T4 phage td gene.

In some embodiments, the 3′ half of Group I catalytic intron fragment and the 5′ Group I catalytic intron fragment are from a T4 phage td gene. The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the Group I catalytic intron fragment is from the T4 phage nrdB gene or nrdD gene. In some embodiments, the 5′ half of Group I catalytic intron fragment is from the T4 phage nrdB gene. In some embodiments, the 3′ half of Group I catalytic intron fragment is from the T4 phage nrdB gene and the 5′ half of Group I catalytic intron fragment is from the T4 phage nrdB gene. In some embodiments, the 5′ half of Group I catalytic intron fragment is from the T4 phage nrdD gene. In some embodiments, the 3′ half of Group I catalytic intron fragment is from the T4 phage nrdD gene and the 5′ half of Group I catalytic intron fragment is from the T4 phage nrdD gene.

The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the 3′ half of Group I catalytic intron fragment is the 5′ terminus of the linear polynucleotide.

In some embodiments, the 5′ half of Group I catalytic intron fragment is the 3′ terminus of the linear polyribonucleotide.

In another example, a linear polyribonucleotide may be cyclized or concatenated by a non-nucleic acid moiety that causes an attraction between atoms, molecular surfaces at, near, or linked to the 5′ and 3′ ends of the linear polyribonucleotide. The one or more linear polyribonucleotides may be cyclized or concatenated by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In another example, the linear polyribonucleotide may include a ribozyme RNA sequence near the 5′ terminus and near the 3′ terminus. The ribozyme RNA sequence may covalently link to a peptide when the sequence is exposed to the remainder of the ribozyme. The peptides covalently linked to the ribozyme RNA sequence near the 5′ terminus and the 3′terminus may associate with each other, thereby causing a linear polyribonucleotide to cyclize or concatenate. In another example, the peptides covalently linked to the ribozyme RNA near the 5′ terminus and the 3′ terminus may cause the linear primary construct or linear mRNA to cyclize or concatenate after being subjected to ligated using various methods known in the art such as, but not limited to, protein ligation. Non-limiting examples of ribozymes for use in the linear primary constructs or linear polyribonucleotides of the present invention or a non-exhaustive listing of methods to incorporate or covalently link peptides are described in US Patent Publication No. US20030082768, the contents of which is here in incorporated by reference in its entirety.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); Muller and Appel, from RNA Biol, 2017, 14(8):1018-1027; and Egli & Herdewijn, Chemistry and Biology of Artificial Nucleic Acids, (First Edition), Wiley-VCH (2012). Other methods of making circular polyribonucleotides are described, for example, in International Publication No. WO2023/044006, International Publication No. WO2022/247943, U.S. Pat. No. 11,000,547, International Publication No. WO2018/191722, International Publication No. WO2019/236673, International Publication No. WO2020/023595, International Publication No. WO2022/204460, International Publication No. WO2022/204464, International Publication No. WO2022/204466, and International Publication No. 2022/261490, the contents of each of which are herein incorporated by reference in their entirety).

Additional methods of synthesizing circular polyribonucleotides are also described elsewhere (see, e.g., U.S. Pat. Nos. 6,210,931, 5,773,244, 5,766,903, 5,712,128, 5,426,180, US Publication No. US20100137407, International Publication No. WO1992001813, International Publication No. WO2010084371, and Petkovic et al., Nucleic Acids Res. 43:2454-65 (2015); the contents of each of which are herein incorporated by reference in their entirety).

In some embodiments, the circular polyribonucleotide is purified, e.g., free ribonucleic acids, linear or nicked RNA, DNA, proteins, etc. are removed. In some embodiments, the circular polyribonucleotides may be purified by any known method commonly used in the art. Examples of nonlimiting purification methods include, column chromatography, gel excision, size exclusion, etc.

### Methods of Production in a Cell-Free System

In some embodiments, circular polyribonucleotides described herein may be produced by transcribing a deoxyribonucleotide template in a cell-free system (e.g., by in vitro transcription) to produce a linear polyribonucleotide. The linear polyribonucleotide produces a splicing-compatible polyribonucleotide, which may be self-spliced to produce a circular polyribonucleotide.

In some embodiments, a circular polyribonucleotide (e.g., in a cell-free system) is produced by providing a linear polyribonucleotide; and self-splicing linear polyribonucleotide under conditions suitable for splicing of the 3′ and 5′ splice sites of the linear polyribonucleotide; thereby producing a circular polyribonucleotide.

In some embodiments, a circular polyribonucleotide is produced by providing a deoxyribonucleotide encoding the linear polyribonucleotide; transcribing the deoxyribonucleotide in a cell-free system to produce the linear polyribonucleotide; optionally purifying the splicing-compatible linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, a circular polyribonucleotide is produced by providing a deoxyribonucleotide encoding a linear polyribonucleotide; transcribing the deoxyribonucleotide in a cell-free system to produce the linear polyribonucleotide, wherein the transcribing occurs in a solution under conditions suitable for splicing of the 3′ and 5′ splice sites of the linear polyribonucleotide, thereby producing a circular polyribonucleotide. In some embodiments, the linear polyribonucleotide comprises a 5′ split-intron and a 3′ split-intron (e.g., a self-splicing construct for producing a circular polyribonucleotide). In some embodiments, the linear polyribonucleotide comprises a 5′ annealing region and a 3′ annealing region.

Suitable conditions for in vitro transcriptions and or self-splicing may include any conditions (e.g., a solution or a buffer, such as an aqueous buffer or solution) that mimic physiological conditions in one or more respects. In some embodiments, suitable conditions include between 0.1-100 mM Mg2+ ions or a salt thereof (e.g., 1-100 mM, 1-50 mM, 1-20 mM, 5-50 mM, 5-20 mM, or 5-15 mM). In some embodiments, suitable conditions include between 1-1000 mM K+ ions or a salt thereof such as KCl (e.g., 1-1000 mM, 1-500 mM, 1-200 mM, 50-500 mM, 100-500 mM, or 100-300 mM). In some embodiments, suitable conditions include between 1-1000 mM Cl− ions or a salt thereof such as KCl (e.g., 1-1000 mM, 1-500 mM, 1-200 mM, 50-500 mM, 100-500 mM, or 100-300 mM). In some embodiments, suitable conditions include between 0.1-100 mM Mn2+ ions or a salt thereof such as MnCl2 (e.g., 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include dithiothreitol (DTT) (e.g., 1-1000 μM, 1-500 μM, 1-200 μM, 50-500 μM, 100-500 μM, 100-300 μM, 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include between 0.1 mM and 100 mM ribonucleoside triphosphate (NTP) (e.g., 0.1-100 mM, 0.1-50 mM, 0.1-10 mM, 1-100 mM, 1-50 mM, or 1-10 mM). In some embodiments, suitable conditions include a pH of 4 to 10 (e.g., pH of 5 to 9, pH of 6 to 9, or pH of 6.5 to 8.5). In some embodiments, suitable conditions include a temperature of 4° C. to 50° C. (e.g., 10° C. to 40° C., 15° C. to 40° C., 20° C. to 40° C., or 30° C. to 40° C.),

In some embodiments the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

### Methods of Production in a Cell

In some embodiments circular polyribonucleotides are produced in a cell, e.g., a prokaryotic cell or a eukaryotic cell. In some embodiments, an exogenous polyribonucleotide is provided to a cell (e.g., a linear polyribonucleotide or a DNA molecule encoding for the transcription of a linear polyribonucleotide). The linear polyribonucleotides may be transcribed in the cell from an exogenous DNA molecule provided to the cell. The linear polyribonucleotide may be transcribed in the cell from an exogenous recombinant DNA molecule transiently provided to the cell. In one embodiment, the linear polyribonucleotide may be transcribed in the cell from an exogenous DNA molecule provided to the cell, such as for example, with a plasmid. In some embodiments, the exogenous DNA molecule does not integrate into the cell's genome. In some embodiments, the linear polyribonucleotide is transcribed in the cell from a recombinant DNA molecule that is incorporated into the cell's genome.

In some embodiments, the cell is a prokaryotic cell. In some embodiments, the prokaryotic cell including the polyribonucleotides described herein may be a bacterial cell or an archaeal cell. For example, the prokaryotic cell including the polyribonucleotides described herein may be E. coli, halophilic archaea (e.g., Haloferax volcanii), Sphingomonas, cyanobacteria (e.g., Synechococcus elongatus, Spirulina (Arthrospira) spp., and Synechocystis spp.), Streptomyces, actinomycetes (e.g., Nonomuraea, Kitasatospora, or Thermobifida), Bacillus spp. (e.g., Bacillus subtilis, Bacillus anthracis, Bacillus cereus), betaproteobacteria (e.g., Burkholderia), alphaproteobacterial (e.g., Agrobacterium), Pseudomonas (e.g., Pseudomonas putida), and enterobacteria. The prokaryotic cells may be grown in a culture medium. The prokaryotic cells may be contained in a bioreactor.

In some embodiments, the cell is a eukaryotic cell. In some embodiments, the eukaryotic cell including the polyribonucleotides described herein is a unicellular eukaryotic cell. In some embodiments, the unicellular eukaryotic is a unicellular fungal cell such as a yeast cell (e.g., Saccharomyces cerevisiae and other Saccharomyces spp., Brettanomyces spp., Schizosaccharomyces spp., Torulaspora spp, and Pichia spp.). In some embodiments, the unicellular eukaryotic cell is a unicellular animal cell. A unicellular animal cell may be a cell isolated from a multicellular animal and grown in culture, or the daughter cells thereof. In some embodiments, the unicellular animal cell may be dedifferentiated. In some embodiments, the unicellular eukaryotic cell is a unicellular plant cell. A unicellular plant cell may be a cell isolated from a multicellular plant and grown in culture, or the daughter cells thereof. In some embodiments, the unicellular plant cell may be dedifferentiated. In some embodiments, the unicellular plant cell is from a plant callus. In embodiments, the unicellular cell is a plant cell protoplast. In some embodiments, the unicellular eukaryotic cell is a unicellular eukaryotic algal cell, such as a unicellular green alga, a diatom, a euglenid, or a dinoflagellate. Non-limiting examples of unicellular eukaryotic algae of interest include Dunaliella salina, Chlorella vulgaris, Chlorella zofingiensis, Haematococcus pluvialis, Neochloris oleoabundans and other Neochloris spp., Protosiphon botryoides, Botryococcus braunii, Cryptococcus spp., Chlamydomonas reinhardtii and other Chlamydomonas spp. In some embodiments, the unicellular eukaryotic cell is a protist cell. In some embodiments, the unicellular eukaryotic cell is a protozoan cell.

In some embodiments, the eukaryotic cell is a cell of a multicellular eukaryote. For example, the multicellular eukaryote may be selected from the group consisting of a vertebrate animal, an invertebrate animal, a multicellular fungus, a multicellular alga, and a multicellular plant. In some embodiments, the eukaryotic organism is a human. In some embodiments, the eukaryotic organism is a non-human vertebrate animal. In some embodiments, the eukaryotic organism is an invertebrate animal. In some embodiments, the eukaryotic organism is a multicellular fungus. In some embodiments, the eukaryotic organism is a multicellular plant. In embodiments, the eukaryotic cell is a cell of a human or a cell of a non-human mammal such as a non-human primate (e.g., monkeys, apes), ungulate (e.g., bovids including cattle, buffalo, bison, sheep, goat, and musk ox; pig; camelids including camel, llama, and alpaca; deer, antelope; and equids including horse and donkey), carnivore (e.g., dog, cat), rodent (e.g., rat, mouse, guinea pig, hamster, squirrel), or lagomorph (e.g., rabbit, hare). In embodiments, the eukaryotic cell is a cell of a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the eukaryotic cell is a cell of an arthropod (e.g., insects, arachnids, crustaceans), a nematode, an annelid, a helminth, or a mollusk. In embodiments, the eukaryotic cell is a cell of a multicellular plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the eukaryotic cell is a cell of a eukaryotic multicellular alga.

The eukaryotic cells may be grown in a culture medium. The eukaryotic cells may be contained in a bioreactor.

### Methods of Purification

One or more purification steps may be included in the methods described herein. For example, in some embodiments, the linear polyribonucleotide is substantively enriched or pure (e.g., purified) prior to self-splicing the linear polyribonucleotide. In other embodiments, the linear polyribonucleotide is not purified prior to self-splicing the linear polyribonucleotide. In some embodiments, the resulting circular polyribonucleotide is purified.

Purification may include separating or enriching the desired reaction product from one or more undesired components, such as any unreacted stating material, byproducts, enzymes, or other reaction components. For example, purification of linear polyribonucleotide following transcription in a cell-free system (e.g., in vitro transcription) may include separation or enrichment from the DNA template prior to self-splicing the linear polyribonucleotide. Purification of the circular polyribonucleotide product following splicing may be used to separate or enrich the circular polyribonucleotide from its corresponding linear polyribonucleotide. Methods of purification of RNA are known to those of skill in the art and include enzymatic purification or by chromatography.

In some embodiments, the methods of purification result in a circular polyribonucleotide that has less than 50% (e.g., less than 40%, 30%, 20%, 10%, 5%, 4%, 3%, 2%, or 1%) linear polyribonucleotides.

### Bioreactors

In some embodiments, any method of producing a circular polyribonucleotide described herein may be performed in a bioreactor. A bioreactor refers to any vessel in which a chemical or biological process is carried out which involves organisms or biochemically active substances derived from such organisms. Bioreactors may be compatible with the cell-free methods for production of circular polyribonucleotides described herein. A vessel for a bioreactor may include a culture flask, a dish, or a bag that may be single use (disposable), autoclavable, or sterilizable. A bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest.

Some methods of the present disclosure are directed to large-scale production of circular polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

In some embodiments, a bioreactor may produce at least 1 g of circular polyribonucleotide. In some embodiments, a bioreactor may produce 1-200 g of circular polyribonucleotide (e.g., 1-10 g, 1-20 g, 1-50 g, 10-50 g, 10-100 g, 50-100 g, or 50-200 g of circular RNA). In some embodiments, the amount produced is measured per liter (e.g., 1-200 g per liter), per batch or reaction (e.g., 1-200 g per batch or reaction), or per unit time (e.g., 1-200 g per hour or per day).

In some embodiments, more than one bioreactor may be utilized in series to increase the production capacity (e.g., one, two, three, four, five, six, seven, eight, or nine bioreactors may be used in series).

### Gene Editing

In some instances, the LNMP/RNA compositions (e.g., mRNA compositions or circRNA compositions) may include a component of a gene editing system. For example, the agent may introduce an alteration (e.g., insertion, deletion (e.g., knockout), translocation, inversion, single point mutation, or other mutation) in a gene in the target organism. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al., Trends Biotechnol. 31(7):397-405, 2013.

Additional descriptions about the component and process of the gene editing system may be found in International Patent Application Publication No. WO 2021/041301, which is incorporated herein by reference in its entirety.

mRNA Therapeutics and circRNA Therapeutics

The LNMP/mRNA composition comprises one or more polynucleotides (e.g., mRNA) encoding one or more antigenic or signaling polypeptides for therapeutic purpose, such as to combat cancer. The one or more polynucleotides (e.g., mRNA) encode one or more cancer (tumor) antigenic polypeptides. In some embodiments, the LNMP/circRNA therapeutic composition comprises one or more circular polyribonucleotides. In some embodiments, the circular polyribonucleotide includes one or more polyribonucleotide (e.g., one or more polyribonucleotide cargo of the polyribonucleotide) encoding one or more antigenic or signaling polypeptides for therapeutic purpose, such as to combat cancer. In some embodiments, the polyribonucleotide includes one or more expression sequences encoding one or more antigenic (e.g., tumor antigenic) or signaling polypeptides.

### Tumor Antigens

Cancers or tumors include but are not limited to neoplasms, malignant tumors, metastases, or any disease or disorder characterized by uncontrolled cell growth such that it would be considered cancerous. The cancer may be a primary or metastatic cancer. Specific cancers that can be treated according to the present invention include, but are not limited to, those listed below (for a review of such disorders, see Fishman et al, 1985, Medicine, 2d Ed., J.B. Lippincott Co., Philadelphia). Cancers include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

In some embodiments, the cancer is selected from the group consisting of non-small cell lung cancer (NSCLC), small cell lung cancer, melanoma, bladder urothelial carcinoma, HPV-negative head and neck squamous cell carcinoma (HNSCC), and a solid malignancy that is microsatellite high (MSI H)/mismatch repair (MMR) deficient. In some embodiments, the NSCLC lacks an EGFR sensitizing mutation and/or an ALK translocation. In some embodiments, the solid malignancy that is microsatellite high (MSI H)/mismatch repair (MMR) deficient is selected from the group consisting of colorectal cancer, stomach adenocarcinoma, esophageal adenocarcinoma, and endometrial cancer. In some embodiments, the cancer is selected from cancer of the pancreas, peritoneum, large intestine, small intestine, biliary tract, lung, endometrium, ovary, genital tract, gastrointestinal tract, cervix, stomach, urinary tract, colon, rectum, and hematopoietic and lymphoid tissues. In some embodiments, the cancer is colorectal cancer.

In some embodiments, the tumor antigenic polypeptide comprises p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, pl90 minor BCR-abL, Plac-1, Pml/RARa, PRAME, proteinase 3, PSA, PSM, RAGE, RU1 or RU2, SAGE, SART-1 or S ART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.

In some embodiments, the tumor antigen is one of the following antigens: CD2, CD3, CD4, CD8, CD11b, CD14, CD16, CD19, CD20, CD22, CD25, CD27, CD33, CD37, CD38, CD40, CD44, CD45, CD47, CD52, CD56, CD70, CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbBI, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gplOO, gpA33, GPNMB, HLA, HLA-DR, ICOS, IGF1R, Integrin av, Integrin avs, LAG-3, Lewis Y, Mesothelin, c-MET, MN Carbonic anhydrase IX, MUC1, MUC16, Nectin-4, KGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof.

In some embodiments, the tumor antigenic polypeptide is IL2 peptide, IL-2-R^{a}, Anti-CD19 antibody, anti-CD20 antibody, chimeric antigen receptor T cell (CAR-T) antibody, Anti-HER2 antibody, etanercept (e.g., Enbrel), adalimumab (e.g., Humira), epoetin alfa (e.g., Epogen), filgrastim (e.g., Neupogen), pembrolizumab (e.g., Keytruda), rituximab (e.g., Rituxan), romiplostim (e.g., Nplate), sargramostim (e.g., Leukine), or a fragment or subunit thereof. In one embodiment, the tumor antigenic polypeptide is IL2 peptide, or a fragment or subunit thereof. In one embodiment, tumor antigenic polypeptide is epoetin alfa (e.g., Epogen), or a fragment or subunit thereof.

In some embodiments, the tumor antigenic polypeptide comprises a tumor antigen selected from the group consisting of a carcinoma, a sarcoma, a melanoma, a lymphoma, a leukemia, and a combination thereof.

In one embodiment, the tumor antigenic polypeptide comprises a melanoma tumor antigen. In one embodiment, the tumor antigenic polypeptide comprises a prostate cancer antigen. In one embodiment, the tumor antigenic polypeptide comprises a HPV16 positive head and neck cancer antigen. In one embodiment, the tumor antigenic polypeptide comprises a breast cancer antigen. In one embodiment, the tumor antigenic polypeptide comprises an ovarian cancer antigen. In one embodiment, the tumor antigenic polypeptide comprises a lung cancer antigen. In one embodiment, the tumor antigenic polypeptide comprises an NSCLC antigen.

In some embodiments, the antigen is a self-antigenic polypeptide or an immunogenic variant or an immunogenic fragment thereof. In some embodiments, a self-antigenic polypeptide comprises an antigen that is typically expressed on cells and is recognized as a self-antigen by an immune system. In some embodiments, the self-antigenic polypeptide comprises: a multiple sclerosis antigenic polypeptide, a Rheumatoid arthritis antigenic polypeptide, a lupus antigenic polypeptide, a celiac disease antigenic polypeptide, a Sjogren's syndrome antigenic polypeptide, or an ankylosing spondylitis antigenic polypeptide, or a combination thereof.

Immune Potentiator mRNAs

In some embodiments, the one or more polynucleotides comprise mRNAs that encode a polypeptide that stimulates or enhances an immune response against one or more of the cancer antigens of interest. Such mRNAs that enhance immune responses to the cancer antigen(s) of interest are referred to herein as immune potentiator mRNA constructs or immune potentiator mRNAs, including chemically modified mRNAs (mmRNAs). In some embodiments, the circular polyribonucleotides disclosed herein comprise one or more expression sequence(s) that encode a polypeptide that stimulates or enhances an immune response against one or more cancer antigens of interest. Such circular polyribonucleotides that enhance immune responses to cancer antigen(s) of interest are referred to herein as immune potentiator circRNAs. An immune potentiator enhances an immune response to an antigen of interest in a subject. The enhanced immune response can be a cellular response, a humoral response or both. As used herein, a “cellular” immune response is intended to encompass immune responses that involve or are mediated by T cells, whereas a “humoral” immune response is intended to encompass immune responses that involve or are mediated by B cells. An immune potentiator may enhance an immune response by, for example,
(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

As used herein, “stimulating Type I interferon pathway signaling” is intended to encompass activating one or more components of the Type I interferon signaling pathway (e.g., modifying phosphorylation, dimerization or the like of such components to thereby activate the pathway), stimulating transcription from an interferon-sensitive response element (ISRE) and/or stimulating production or secretion of Type I interferon (e.g., IFN-a, IFN-β, IFN-ε, IFN-K and/or IFN-co). As used herein, “stimulating NFkB pathway signaling” is intended to encompass activating one or more components of the NFkB signaling pathway (e.g., modifying phosphorylation, dimerization or the like of such components to thereby activate the pathway), stimulating transcription from an NFkB site and/or stimulating production of a gene product whose expression is regulated by NFkB. As used herein, “stimulating an inflammatory response” is intended to encompass stimulating the production of inflammatory cytokines (including but not limited to Type I interferons, IL-6 and/or TNFa). As used herein, “stimulating dendritic cell development, activity or mobilization” is intended to encompass directly or indirectly stimulating dendritic cell maturation, proliferation and/or functional activity.

In some embodiments, the mRNA or circRNA encodes a polypeptide that stimulates or enhances an immune response in a subject in need thereof (e.g., potentiates an immune response in the subject) by, for example, inducing adaptive immunity (e.g., by stimulating Type I interferon production), stimulating an inflammatory response, stimulating FkB signaling and/or stimulating dendritic cell (DC) development, activity or mobilization in the subject. In some embodiments, administration of an immune potentiator mRNA or circRNA to a subject in need thereof enhances cellular immunity (e.g., T cell-mediated immunity), humoral immunity (e.g., B cell-mediated immunity) or both cellular and humoral immunity in the subject. In some embodiments, administration of an immune potentiator mRNA or circRNA stimulates cytokine production (e.g., inflammatory cytokine production), stimulates cancer antigen-specific CD8⁺ effector cell responses, stimulates antigen-specific CD4⁺ helper cell responses, increases the effector memory CD62L¹⁰ T cell population, stimulates B cell activity or stimulates antigen-specific antibody production, including combinations of the foregoing responses. In some embodiments, administration of an immune potentiator mRNA or circRNA stimulates cytokine production (e.g., inflammatory cytokine production) and stimulates antigen-specific CD8⁺ effector cell responses. In some embodiments, administration of an immune potentiator mRNA or circRNA stimulates cytokine production (e.g., inflammatory cytokine production), and stimulates antigen-specific CD4⁺ helper cell responses. In some embodiments, administration of an immune potentiator mRNA or circRNA stimulates cytokine production (e.g., inflammatory cytokine production), and increases the effector memory CD62L¹⁰ T cell population. In some embodiments, administration of an immune potentiator mRNA or circRNA stimulates cytokine production (e.g., inflammatory cytokine production), and stimulates B cell activity or stimulates antigen-specific antibody production.

In one embodiment, an immune potentiator increases cancer antigen-specific CD8⁺ effector cell responses (cellular immunity). For example, an immune potentiator can increase one or more indicators of antigen-specific CD8⁺ effector cell activity, including but not limited to CD8+ T cell proliferation and CD8+ T cell cytokine production. For example, in one embodiment, an immune potentiator increases production of IFN-γ, TNFa and/or IL-2 by antigen-specific CD8+ T cells. In various embodiments, an immune potentiator can increase CD8+ T cell cytokine production (e.g., IFN-γ, TNFa and/or IL-2 production) in response to an antigen (as compared to CD8+ T cell cytokine production in the absence of the immune potentiator) by at least 5% or at least 10% or at least 15% or at least 20% or at least 25% or at least 30%) or at least 35% or at least 40% or at least 45% or at least 50%. For example, T cells obtained from a treated subject can be stimulated in vitro with the cancer antigens and CD8+ T cell cytokine production can be assessed in vitro. CD8+ T cell cytokine production can be determined by standard methods known in the art, including but not limited to measurement of secreted levels of cytokine production (e.g., by ELISA or other suitable method known in the art for determining the amount of a cytokine in supernatant) and/or determination of the percentage of CD8+ T cells that are positive for intracellular staining (ICS) for the cytokine. For example, intracellular staining (ICS) of CD8+ T cells for expression of IFN-γ, TNFα and/or JL-2 can be carried out by methods known in the art. In one embodiment, an immune potentiator increases the percentage of CD8+ T cells that are positive by ICS for one or more cytokines (e.g., IFN-γ, TNFa and/or IL-2) in response to an antigen (as compared to the percentage of CD8+ T cells that are positive by ICS for the cytokine(s) in the absence of the immune potentiator) by at least 5% or at least 10% or at least 15%>or at least 20% or at least 25% or at least 30%>or at least 35% or at least 40% or at least 45% or at least 50%.

In one embodiment, an immune potentiator increases the percentage of CD8+ T cells among the total T cell population (e.g., splenic T cells and/or PBMCs), as compared to the percentage of CD8+ T cells in the absence of the immune potentiator. For example, an immune potentiator can increase the percentage of CD8+ T cells among the total T cell population by at least 5% or at least 10% or at least 15% or at least 20% or at least 25% or at least 30%) or at least 35% or at least 40% or at least 45% or at least 50%, as compared to the percentage of CD8+ T cells in the absence of the immune potentiator. The total percentage of CD8+ T cells among the total T cell population can be determined by standard methods known in the art, including but not limited to fluorescent activated cell sorting (FACS) or magnetic activated cell sorting (MACS).

In one embodiment, an immune potentiator increases a tumor-specific immune cell response, as determined by a decrease in tumor volume in vivo in the presence of the immune potentiator as compared to tumor volume in the absence of the immune potentiator. For example, an immune potentiator can decrease tumor volume by at least 5% or at least 10%) or at least 15% or at least 20% or at least 25% or at least 30% or at least 35% or at least 40%) or at least 45% or at least 50%, as compared to tumor volume in the absence of the immune potentiator. Measurement of tumor volume can be determined by methods well established in the art. In another embodiment, an immune potentiator increases B cell activity (humoral immune response), for example by increasing the amount of antigen-specific antibody production, as compared to antigen-specific antibody production in the absence of the immune potentiator. For example, an immune potentiator can increase antigen-specific antibody production by at least 5% or at least 10% or at least 15% or at least 20% or at least 25%) or at least 30%>or at least 35% or at least 40% or at least 45% or at least 50%, as compared to antigen-specific antibody production in the absence of the immune potentiator. In one embodiment, antigen-specific IgG production is evaluated. Antigen-specific antibody production can be evaluated by methods well established in the art, including but not limited to ELISA, RIA and the like that measure the level of antigen-specific antibody (e.g., IgG) in a sample (e.g., a serum sample).

In one embodiment, an immune potentiator increases the effector memory CD62L¹⁰ T cell population. For example, an immune potentiator can increase the total % of CD62L¹⁰ T cells among CD8+ T cells. Among other functions, the effector memory CD62L¹⁰ T cell population has been shown to have an important function in lymphocyte trafficking (see e.g., Schenkel, J. M. and Masopust, D. (2014) Immunity 41:886-897). In various embodiments, an immune potentiator can increase the total percentage of effector memory CD62L¹⁰ T cells among the CD8+ T cells in response to an antigen (as compared to the total percentage of CD62L¹⁰ T cells among the CD8+ T cells population in the absence of the immune potentiator) by at least 5% or at least 10% or at least 15% or at least 20% or at least 25% or at least 30% or at least 35% or at least 40% or at least 45% or at least 50%. The total percentage of effector memory CD62L¹⁰ T cells among the CD8+ T cells can be determined by standard methods known in the art, including but not limited to fluorescent activated cell sorting (FACS) or magnetic activated cell sorting (MACS).

The ability of an immune potentiator mRNA or circRNA to enhance an immune response to a cancer antigen can be evaluated in mouse model systems known in the art. In one embodiment, an immune competent mouse model system is used. In one embodiment, the mouse model system comprises C57/B16 mice. In another embodiment, the mouse model system comprises BalbC mice or CD1 mice {e.g., to evaluate B cell responses, such an antigen-specific antibody responses). In one embodiment, an immune potentiator polypeptide functions downstream of at least one Toll-like receptor (TLR) to thereby enhance an immune response. Accordingly, in one embodiment, the immune potentiator is not a TLR but is a molecule within a TLR signaling pathway downstream from the receptor itself.

In one embodiment, the mRNA or circRNA encoding an immune potentiator can comprises one or more modified nucleobases. Suitable modifications are discussed further below.

In one embodiment, the mRNA or circRNA encoding an immune potentiator is formulated into the LNMP formulation. In one embodiment, the mRNA or circRNA encodes a cancer antigen. In one embodiment, the LNMP/mRNA formulation or LNMP/circRNA formulation is administered to a subject to enhance an immune response against the cancer antigen in the subject.

Immune Potentiator mRNAs that Stimulate Type I Interferon

In some embodiments, the disclosure provides an immune potentiator mRNA or circRNA encoding a polypeptide that stimulates or enhances an immune response against an antigen of interest by simulating or enhancing Type I interferon pathway signaling, thereby stimulating or enhancing Type I interferon (IFN) production.

A number of components involved in Type I IFN pathway signaling have been established, including STING, Interferon Regulatory Factors, such as IRF1, IRF3, IRF5, IRF7, IRF8, and IRF9, TBK1, IKKi, MyD88 and TRAM. Additional components involved in Type I IFN pathway signaling include TRAF3, TRAF6, IRAK-1, IRAK-4, TRIF, IPS-1, TLR-3, TLR-4, TLR-7, TLR-8, TLR-9, RIG-1, DAI, and IF116.

Accordingly, in one embodiment, an immune potentiator mRNA or circRNA encodes any of the foregoing components involved in Type I IFN pathway signaling.

### Agents for Promotion of Antigen Presenting Cells

In some embodiments, LNMP/RNA composition (e.g., mRNA composition or circRNA composition) can be combined with agents for promoting the production of antigen presenting cells (APCs), for instance, by converting non-APCs into pseudo-APCs. Antigen presentation is a key step in the initiation, amplification and duration of an immune response. In this process fragments of antigens are presented through the

Major Histocompatibility Complex (MHC) or Human Leukocyte Antigens (HLA) to T cells driving an antigen-specific immune response. For immune prophylaxis and therapy, enhancing this response is important for improved efficacy. The LNMP/RNA composition (e.g., mRNA composition or circRNA composition) may be designed or enhanced to drive efficient antigen presentation. One method for enhancing APC processing and presentation, is to provide better targeting of the LNMP/RNA composition (e.g., mRNA composition or circRNA composition) to antigen presenting cells (APC). Another approach involves activating the APC cells with immune-stimulatory formulations and/or components. Alternatively, methods for reprograming non-APC into becoming APC may be used with the LNMP/RNA composition (e.g., mRNA composition or circRNA composition). Importantly, most cells that take up mRNA formulations and are targets of their therapeutic actions are not APC. Therefore, designing a way to convert these cells into APC would be beneficial for efficacy. Methods and approaches for delivering the LNMP/RNA composition (e.g., mRNA composition or circRNA composition), e.g., mRNA or circRNA vaccines to cells while also promoting the shift of a non-APC to an APC are provided herein. In some embodiments, a mRNA or circRNA encoding an APC reprograming molecule is included in the LNMP/RNA composition (e.g., mRNA composition or circRNA composition) or coadministered with the LNMP/RNA composition (e.g., mRNA composition or circRNA composition).

An APC reprograming molecule, as used herein, is a molecule that promotes a transition in a non-APC cell to an APC-like phenotype. An APC-like phenotype is property that enables MHC class II processing. Thus, an APC cell having an APC-like phenotype is a cell having one or more exogenous molecules (APC reprograming molecule) which has enhanced MHC class II processing capabilities in comparison to the same cell not having the one or more exogenous molecules. In some embodiments, an APC reprograming molecule is a CUT A (a central regulator of MHC Class II expression); a chaperone protein such as CLIP, HLA-DO, HLA-DM etc. (enhancers of loading of antigen fragments into MHC Class II) and/or a costimulatory molecule like CD40, CD80, CD86 etc. (enhancers of T cell antigen recognition and T cell activation).

A CIITA protein is a transactivator that enhances activation of transcription of MHC Class II genes (Steimle et al., 1993, Cell 75: 135-146) by interacting with a conserved set of DNA binding proteins that associate with the class II promoter region. The transcriptional activation function of CIITA has been mapped to an amino terminal acidic domain (amino acids 26-137). A nucleic acid molecule encoding a protein that interacts with CIITA, termed CIITA-interacting protein 104 (also referred to herein as CIP104). Both CITTA and CIP104 have been shown to enhance transcription from MHC class II promoters and thus are useful as APC reprograming molecule of the invention. In some embodiments the APC reprograming molecule are full length CIITA, CIP 104 or other related molecules or active fragments thereof, such as amino acids 26-137 of CIITA, or amino acids having at least 80% sequence identity thereto and maintaining the ability to enhance activation of transcription of MHC Class II genes.

In some embodiments, the LNMP/RNA composition (e.g., mRNA composition or circRNA composition) may include a recall antigen, also sometimes referred to as a memory antigen. A recall antigen is an antigen that has previously been encountered by an individual and for which there are pre-existent memory lymphocytes. In some embodiments, the recall antigen may be an infectious disease antigen that the individual has likely encountered such as an influenza antigen. The recall antigen helps promote a more robust immune response.

The antigens or neoepitopes selected for inclusion in the LNMP/RNA composition (e.g., mRNA composition or circRNA composition) typically will be high affinity binding peptides. In some embodiments, the antigens or neoepitopes binds an HLA protein with greater affinity than a wild-type peptide. The antigen or neoepitope has an IC50 of at least less than 5000 nM, at least less than 500 nM, at least less than 250 nM, at least less than 200 nM, at least less than 150 nM, at least less than 100 nM, at least less than 50 nM or less in some embodiments. Typically, peptides with predicted IC50<50 nM, are generally considered medium to high affinity binding peptides and will be selected for testing their affinity empirically using biochemical assays of HLA-binding.

The cancer antigens can be personalized cancer antigens. The LNMP/RNA composition (e.g., mRNA composition or circRNA composition) may include RNA encoding for one or more known cancer antigens specific for the tumor or cancer antigens specific for each subject, referred to as neoepitopes or subject specific epitopes or antigens. A “subject specific cancer antigen” is an antigen that has been identified as being expressed in a tumor of a particular patient. The subject specific cancer antigen may or may not be typically present in tumor samples generally. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes, like tumor associated antigens, are completely foreign to the body and thus would not produce an immune response against healthy tissue or be masked by the protective components of the immune system. In some embodiments, the LNMP/RNA composition (e.g., mRNA compositions or circRNA composition) based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and translocations.

Thus, in some embodiments, the LNMP/RNA composition (e.g., mRNA compositions or circRNA composition) include at least 1 cancer antigens including mutations selected from the group consisting of frame-shift mutations and recombinations or any of the other mutations described herein.

### Nucleic Acid Sequences

In some embodiments, the polynucleotides are polynucleotide constructs, which encode one or more wild type or engineered antigens (or an antibody to an antigen). In some embodiments, the polynucleotide comprises a tumor antigenic polypeptide or an immunogenic variant or an immunogenic fragment thereof. The antigen may be derived from a tumor, e.g., a tumor specific antigen, a tumor associated antigen, a tumor neoantigen, or a combination thereof.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA.

In some embodiments, the polyribonucleotide comprises a plurality of sequences, each encoding an antigen, and at least one sequence of the plurality encodes a coronavirus antigen. In some embodiments, the circular polyribonucleotide comprises two or more ORFs. In some embodiments, the circular polyribonucleotide comprises at least five sequences, each encoding an antigen and at least one of the antigens is a coronavirus antigen. In some embodiments, the circular polyribonucleotide comprises at least two ORFs, e.g., at least 2, 3, 4 or 5 ORFs. In some embodiments, the circular polyribonucleotide comprises between 5 and 20 sequences, each encoding an antigen and at least one of the antigens is a coronavirus antigen. In some embodiments, the circular polyribonucleotide comprises between 5 and 10 sequences, each encoding an antigen and at least one of the antigens is a coronavirus antigen. In some embodiments, the circular polyribonucleotide comprises sequences encoding antigens from at least two different microorganisms, and at least one microorganism is a coronavirus.

In some embodiments, the circular polyribonucleotide comprises one or more of the elements as described herein in addition to comprising a binding site (e.g., a sequence for binding to a target). In some embodiments, the circular polyribonucleotide lacks a poly-A tail. In some embodiments, the circular polyribonucleotide lacks a replication element. In some embodiments, the circular polyribonucleotide lacks an IRES. In some embodiments, the circular polyribonucleotide lacks a cap. In some embodiments, the circular polyribonucleotide comprises any feature or any combination of features as disclosed in WO2019/118919 in addition to a binding site, which is hereby incorporated by reference in its entirety.

In some embodiments, a circRNA can comprise at least one binding site for a target, e.g., for a binding moiety of a target. A circRNA can comprise at least one aptamer sequence that binds to a target. In some embodiments, the circRNA comprises one or more binding sites for one or more targets. Targets include, but are not limited to, nucleic acids (e.g., RNAs, DNAs, RNA-DNA hybrids), small molecules (e.g., drugs, fluorophores, metabolites), aptamers, polypeptides, proteins, lipids, carbohydrates, antibodies, viruses, virus particles, membranes, multi-component complexes, organelles, cells, other cellular moieties, any fragments thereof, and any combination thereof. (See, e.g., Fredriksson et al., (2002) Nat Biotech 20:473-77; Gullberg et al, (2004) PNAS, 101:8420-24). For example, a target is a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, a lipid, a carbohydrate, an antibody, an antibody fragment, a mixture of antibodies, a virus particle, a membrane, a multi-component complex, a cell, a cellular moiety, any fragment thereof, or any combination thereof.

In some embodiments, any of the circular polynucleotide described herein can be prepared according to International Patent Publication No. WO2019118919, WO2020198403, WO2021236952, WO2021236980, WO2021189059, WO2021236855, WO2021226597, and WO2021113777 which are incorporated herein by reference in their entirety.

In some embodiments, the polynucleotide encodes a tumor antigenic polypeptide comprising p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1 R, Myosin/m, MUC1, MUM-1, -2, -3, NA88-A, NF1, NY-ESO-1, NY-BR-1, p190 minor BCR-abL, Plac-1, Pml/RARa, PRAME, proteinase 3, PSA, PSM, RAGE, RU1 or RU2, SAGE, SART-1 or S ART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.

In some embodiments, the polynucleotide encodes a IL2 peptide, IL-2-R^{a}, Anti-CD19 antibody, anti-CD20 antibody, chimeric antigen receptor T cell (CAR-T) antibody, anti-HER2 antibody, etanercept (e.g., Enbrel), adalimumab (e.g., Humira), epoetin alfa (e.g., Epogen), filgrastim (e.g., Neupogen), pembrolizumab (e.g., Keytruda), rituximab (e.g., Rituxan), romiplostim (e.g., Nplate), sargramostim (e.g., Leukine), or a variant, fragment or subunit thereof. Exemplary sequences include those shown in Table 3.

Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence that is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

In some embodiments, the polynucleotide is an mRNA. Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

In some embodiments, the polynucleotide (e.g., mRNA) having an open reading frame (ORF) encoding a cancer antigen. An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. The sequences may further comprise additional elements, e.g., 5′ and 3′ UTRs.

In some embodiments, the RNA (e.g., mRNA) further comprises a 5′ UTR, 3′ UTR, a poly(A) tail and/or a 5′ cap analog.

In some embodiments, the mRNA comprises a 5′ untranslated region (UTR) and/or a 3′ UTR.

In some embodiments, the mRNA is (a) a DNA molecule; or (b) an RNA molecule. In the mRNA, T is optionally substituted with U.

In some embodiments, the mRNA is a DNA molecule. The DNA molecule can further comprise a promoter. In some embodiments, the promoter is a T7 promoter, a T3 promoter, or an SP6 promoter. In some embodiments, the promoter is located at the 5′ UTR.

In some embodiments, the mRNA is an RNA molecule. The RNA molecule may be a self-replicating RNA molecule.

In some embodiments, the mRNA is an RNA molecule. The RNA molecule may further comprise a 5′ cap. The 5′ cap can have a Cap 1 structure, a Cap 1 (m6A) structure, a Cap 2 structure, a Cap 3 structure, a Cap 0 structure, or any combination thereof.

In some embodiments, the polynucleotide is an mRNA which encodes an IL-2 molecule. In one embodiment, the IL-2 molecule comprises a naturally occurring IL-2 molecule, a fragment of a naturally occurring IL-2 molecule, or a variant thereof. In one embodiment, the IL-2 molecule comprises a variant of a naturally occurring IL-2 molecule (e.g., an IL-2 variant, e.g., as described herein), or a fragment thereof.

In one embodiment, the polynucleotide is an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables I-III.

In some embodiments, the mRNA comprises a 5′ untranslated region (UTR) and/or a 3′ UTR.

In some embodiments, the mRNA comprises a 5′ UTR. The 5′ UTR may comprise a Kozak sequence.

In some embodiments, the mRNA comprises a 3′ UTR. In some embodiments, the 3′ UTR comprises one or more sequences derived from an amino-terminal enhancer of split (AES). In some embodiments, the 3′ UTR comprises a sequence derived from mitochondrially encoded 12S mRNA (mtRNRI).

In some embodiments, the mRNA comprises a poly(A) sequence. In one embodiment, the poly(A) sequence is a 110-nucleotide sequence consisting of a sequence of 30 adenosine residues, a 10-nucleotide linker sequence, and a sequence of 70 adenosine residues.

TABLE V
Exemplary IL-2 sequences, human serum albumin (HSA)
sequences and HSA-IL-2 sequences

| SEQ | | |
|---|---|---|
| ID | Sequence | |
| NO | information | Sequence |
| 1 | Human IL-2 | APTSSSTKKTQLQLEHLLLDLQMILNGINNYKNPKLTRMLTFKF |
| | | YMPKKATELKHLQCLEEELKPLEEVLNLAQSKNFHLRPRDLIS |
| | | NINVIVLELKGSETTFMCEYADETATIVEFLNRWITFCQSIISTLT |
| 30 | Human IL-2 | MYRMQLLSCIALSLALVTNSAPTSSSTKKTQLQLEHLLLDLQMI |
| | | LNGINNYKNPKLTRMLTFKFYMPKKATELKHLQCLEEELKPLE |
| | | EVLNLAQSKNFHLRPRDLISNINVIVLELKGSETTEMCEYADET |
| | | ATIVEFLNRWITFCQSIISTLT |
| 2 | Human IL-2 | APTSSSTKKTQLQLEHLLLDLQMILNGINNYKNPKLTRMLTEKF |
| | N88D | YMPKKATELKHLQCLEEELKPLBEVLNLAQSKNFHLRPRDLIS |
| | | DINVIVLELKGSETTEMCEYADETATIVEFLNRWITFCQSIISTLT |
| 31 | Human IL-2 | MYRMQLLSCIALSLALVTNSAPTSSSTKKTQLQLEHLLLDLQMI |
| | N88D | LNGINNYKNPKLTRMLTFKFYMPKKATELKHLQCLEEELKPLE |
| | | EVLNLAQSKNFHLRPRDLISDINVIVLELKGSETTFMCEYADET |
| | | ATIVEFLNRWITFCQSIISTLT |
| 3 | Human IL-2 | APTSSSTKKTQLQLEHLLLDLQMILNGINNYKNPKLTRMLTFKF |
| | V69A, Q74P, | YMPKKATELKHLQCLEEELKPLEEALNLAPSKNFHLRPRDLIS |
| | N88D | DINVIVLELKGSETTEMCEYADETATIVEFLNRWITFCQSIISTLT |
| 32 | Human IL-2 | MYRMQLLSCIALSLALVTNSAPTSSSTKKTQLQLEHLLLDLQMI |
| | V69A, Q74P, | LNGINNYKNPKLTRMLTFKFYMPKKATELKHLQCLEEELKPLE |
| | N88D | EALNLAPSKNFHLRPRDLISDINVIVLELKGSETTFMCEYADET |
| | | ATIVEFLNRWITFCQSIISTLT |
| 4 | Human IL-2, | APTSSSTKKTQLQLEHLLLDLQMILNGINNYKNPKLTRMLTFKF |
| | V91K | YMPKKATELKHLQCLEEELKPLEEVINLAQSKNFHLRPRDLIS |
| | | NINKIVLELKGSETTEMCEYADETATIVEFLNRWITFCQSUSTLT |
| 33 | Human IL-2, | MYRMQLLSCIALSLALVTNSAPTSSSTKKTQLQLEHLLLDLQMI |
| | V9IK | LNGINNYKNPKLTRMLTFKFYMPKKATELKHLQCLEEELKPLE |
| | | EVLNLAQSKNFHLRPRDLISNINKIVLELKGSETTEMCEYADET |
| | | ATIVEFLNRWITFCQSIISTLT |
| 5 | Human IL-2 | APTSSSTKKTQLQLEHLLLDLQMILNGINNYKNPKLTRMLTFKF |
| | V69A, Q74P | YMPKKATELKHLQCLEEELKPLEEALNLAPSKNFHLRPRDLIS |
| | V91K | NINKIVLELKGSETTFMCEYADETATIVEELNRWITFCQSIISTLT |
| 34 | Human IL-2 | MYRMQLLSCIALSLALVTNSAPTSSSTKKTQLQLEHLLLDLQMI |
| | V69A, Q74P, | LNGINNYKNPKLTRMLTPKFYMPKKATELKHLQCLEEELKPLE |
| | V91K | EALNLAPSKNFHLRPRDLISNINKIVLELKGSETTFMCEYADET |
| | | ATIVEFLNRWITFCQSIISTLT |
| 6 | Human IL-2 | APTSSSTKKTQLQLEHLLLTLQMILNGINNYKNPKLTRMLTFKF |
| | D20T | YMPKKATELKHLQCLEEELKPLEEVLNLAQSKNFHLRPRDLIS |
| | | NINVIVLELKGSETTFMCEYADETATIVEFLNRWITFCQSIISTLT |
| 35 | Human IL-2 | MYRMQLLSCIALSLALVTNSAPTSSSTKKTQLQLEHLLLTLQMI |
| | D20T | LNGINNYKNPKLTRMLTFKFYMPKKATELKHLQCLEEELKPLE |
| | | EVLNLAQSKNFHLRPRDLISNINVIVLELKGSETTEMCEYADET |
| | | ATIVEFLNRWITFCQSHISTLT |
| 7 | Human IL-2 | AGUUCCCUAU CACUCUCUUU AAUCACUACU |
| | mRNA | CACAGUAACC UCAACUCCUG CCACAAUGUA |
| | sequence | CAGGAUGCAA CUCCUGUCUU GCAUUGCACU |
| | | AAGUCUUGCA CUUGUCACAA ACAGUGCACC |
| | | UACUUCAAGU UCUACAAAGA AAACACAGCU |
| | | ACAACUGGAG CAUUUACUGC UGGAUUUACA |
| | | GAUGAUUUUG AAUGGAAUUA AUAAUUACAA |
| | | GAAUCCCAAA CUCACCAGGA UGCUCACAUU |
| | | UAAGUUUUAC AUGCCCAAGA AGGCCACAGA |
| | | ACUGAAACAU CUUCAGUGUC UAGAAGAAGA |
| | | ACUCAAACCU CUGGAGGAAG UGCUAAAUUU |
| | | AGCUCAAAGC AAAAACUUUC ACUUAAGACC |
| | | CAGGGACUUA AUCAGCAAUA UCAACGUAAU |
| | | AGUUCUGGAA CUAAAGGGAU CUGAAACAAC |
| | | AUUCAUGUGU GAAUAUGCUG AUGAGACAGC |
| | | AACCAUUGUA GAAUUUCUGA ACAGAUGGAU |
| | | UACCUUUUGU CAAAGCAUCA UCUCAACACU |
| | | GACUUGAUAA UUAAGUGCUU CCCACUUAAA |
| | | ACAUAUCAGG CCUUCUAUUU AUUUAAAUAU |
| | | UUAAAUUUUA UAUUUAUUGU UGAAUGUAUG |
| | | GUUUGCUACC UAUUGUAACU AUUAUUCUUA |
| | | AUCUUAAAAC UAUAAAUAUG GAUCUUUUAU |
| | | GAUUCUUUUU GUAAGCCCUA GGGGCUCUAA |
| | | AAUGGUUUCA CUUAUUUAUC CCAAAAUAUU |
| | | UAUUAUUAUG UUGAAUGUUA AAUAUAGUAU |
| | | CUAUGUAGAU UGGUUAGUAA AACUAUUUAA |
| | | UAAAUUUGAU AAAUAUAAAA AAAAAAAAAA |
| | | AAAAAAAAAA AA |
| 8 | Human | AHKSEVAHREKDLGEENFKALVLIAFAQYLQQCPFEDHVKLVN |
| | serum | EVTEFAKTCVADESAENCDKSLHTLFGDKLCTVATLRETYGE |
| | albumin | MADCCAKQEPERNECFLQHKDDNPNLPRLVRPEVDVMCTAFH |
| | (HSA) | DNEETFLKKYLYEIARRHPYFYAPELLFFAKRYKAAFTECCQA |
| | | ADKAACLLPKLDELRDEGKASSAKQRLKCASLQKFGERAFKA |
| | | WAVARLSQRFPKABFABVSKLVTDLTKVHTECCHGDLLECAD |
| | | DRADLAKYICENQDSISSKLKECCEKPLLEKSHCIAEVENDEMP |
| | | ADLPSLAADEVESKDVCKNYAEAKDVFLGMFLYEYARRHPDY |
| | | SVVLLLRLAKTYETTLEKCCAAADPHECYAKVFDEFKPLVEEP |
| | | QNLIKQNCELFEQLGEYKFQNALLVRYTKKVPQVSTPTLVEVS |
| | | RNLGKVGSKCCKHPEAKRMPCAEDYLSVVLNQLCVLHEKTPV |
| | | SDRVTKCCTESLVNRRPCFSALEVDETYVPKEFNAETFTFHADI |
| | | CTLSEKERQIKKQTALVELVKHKPKATKEQLKAVMDDFAAFV |
| | | EKCCKADDKETCFABEGKKLVAASQAALGL |
| 9 | Leader- | MKWVTFISLLFLFSSAYSRGVFRRDAHKSEVAHRFKDLGEEN |
| | HSA-(G3S)- | FKALVLIAFAQYLQQCPFEDHVKLVNEVTEFAKTCVADESAEN |
| | (Human | CDKSLHTLFGDKLCTVATLRETYGEMADCCAKQEPERNECFL |
| | IL-2) | QHKDDNPNLPRLVRPEVDVMCTAFHDNEETFLKKYLYEIARR |
| | | HPYFYAPELLFFAKRYKAAFTECCQAADKAACLLPKLDELRDE |
| | | GKASSAKQRLKCASLQKFGERAFKAWAVARLSQRFPKABFAE |
| | | VSKLVTDLTKVHTECCHGDLLECADDRADLAKYICENQDSISS |
| | | KLKECCEKPLLEKSHCIAEVENDEMPADLPSLAADFVESKDVC |
| | | KNYAEAKDVFLGMFLYEYARRHPDYSVVLLLRLAKTYETTLE |
| | | KCCAAADPHECYAKVFDEFKPLVEEPQNLIKQNCELFEQLGEY |
| | | KFQNALLVRYTKKVPQVSTPTLVEVSRNLGKVGSKCCKHPEA |
| | | KRMPCAEDYLSVVLNQLCVLHEKTPVSDRVTKCCTESLVNRR |
| | | PCFSALEVDETYVPKEFNAETFTFHADICTLSEKERQIKKQTAL |
| | | VELVKHKPKATKEQLKAVMDDFAAFVEKCCKADDKETCFAE |
| | | EGKKLVAASQAALGLGGGSAPTSSSTKKTQLQLEHLLLDLQMI |
| | | LNGINNYKNPKLTRMLTFKFYMPKKATELKHLQCLEEELKPLE |
| | | EVLNLAQSKNFHLRPRDLISNINVIVLELKGSETTEMCEYADET |
| | | ATIVEFLNRWITFCQSIISTLT |
| 10 | Leader- | MKWVTFISLLFLFSSAYSRGVERRDAHKSEVAHRFKDLGEE |
| | HSA-(G3S) | NFKALVLIAFAQYLQQCPFEDHVKLVNEVTEFAKTCVADESAE |
| | (Human | NCDKSLHTLFGDKLCTVATLRETYGEMADCCAKQEPERNECF |
| | IL-2 N88D) | LQHKDDNPNLPRLVRPEVDVMCTAFHDNEETFLKKYLYEIAR |
| | | RHPYFYAPELLFFAKRYKAAFTECCQAADKAACLLPKLDELR |
| | | DEGKASSAKQRLECASLQKFGERAFKAWAVARLSQRFPKAEF |
| | | AEVSKLVTDLIKVHTECCHGDLLECADDRADLAKYICENQDSI |
| | | SSKLKECCEKPLLEKSHCIABVENDEMPADLPSLAADFVESKD |
| | | VCKNYAEAKDVFLGMFLYEYARRHPDYSVVLLLRLAKTYETT |
| | | LEKCCAAADPHECYAKVEDEFKPLVEEPQNLIKQNCELFEQLG |
| | | EYKFQNALLVRYTKKVPQVSTPTLVEVSRNLGKVGSKCCKHP |
| | | EAKRMPCAEDYLSVVLNQLCVLHEKTPVSDRVTKCCTESLVN |
| | | RRPCFSALEVDETYVPKEFNAETFTFHADICTLSEKERQIKKQT |
| | | ALVELVKHKPKATKBQLKAVMDDFAAFVEKCCKADDKETCF |
| | | AEEGKKLVAASQAALGLGGGSAPTSSSIKKTQLQLEHLLLDLQ |
| | | MILNGINNYKNPKLTRMLTFKFYMPKKATELKHLQCLEEELKP |
| | | LEEVLNLAQSKNFHLRPRDLISDINVIVLELKGSETTEMCEYAD |
| | | ETATIVEFLNRWITFCQSIISTLT |
| 11 | Leader- | MKWVTFISLLFLFSSAYSRGVFRRDAHKSEVAHREFKDLGEEN |
| | HSA-(G3S)- | FKALVLIAFAQYLQQCPFEDHVKLVNEVTEFAKTCVADESAEN |
| | (Human | CDKSHTLFGDKLCTVATLRETYGEMADCCAKQEPERNECFL |
| | IL-2 V69A, | QHKDDNPNLPRLVRPEVDVMCTAFHDNEETFLKKYLYEIARR |
| | Q74P, | HPYFYAPELLFFAKRYKAAFTECCQAADKAACLLPKLDELRDE |
| | N88D) | GKASSAKQRLKCASLQKFGERAFKAWAVARLSQREPKAEFAE |
| | | VSKLVTDLTKVHTECCHGDLLECADDRADLAKYICENQDSISS |
| | | KLKECCEKPLLEKSHCIAEVENDEMPADLPSLAADFVESKDVC |
| | | KNYAEAKDVFLGMFLYEYARRHPDYSVVILLRLAKTYBTTLE |
| | | KCCAAADPHECYAKVEDEFKPLVEEPQNLIKQNCELFEQLGEY |
| | | KFQNALLVRYTKKVPQVSTPTLVEVSRNLGKVGSKQCKHPEA |
| | | KRMPCAEDYLSVVLNQLCVLHEKTPVSDRVTKCCTESLVNRR |
| | | PCFSALEVDETYVPKEFNAETFTFHADICTLSEKERQIKKQTAL |
| | | VELVKHKPKATKEQLKAVMDDFAAFVEKCCKADDKETCFAE |
| | | EGKKLVAASQAALGLGGGSAPTSSSTKKTQLQLEHLLLDLQMI |
| | | LNGINNYKNPKLTRMLTEKFYMPKKATELKHLQCLEEELKPLE |
| | | EALNLAPSKNEHIRPRDLISDINVIVLELKGSETTEMCEYADET |
| | | ATIVEELNRWITECQSIISTLT |
| 36 | Leader- | AUGAAGUGGGUGACCUUCAUCAGCCUGCUGUUCCUGUUCA |
| | HSA-(G3S) | GCAGCGCCUACAGCAGAGGCGUGUUCAGAAGAGACGCCCA |
| | (Human | CAAGAGCGAGGUGGCCCACAGAUUCAAGGACCUGGGCGAG |
| | IL-2 V69A, | GAGAACUUCAAGGCCCUGGUGCUGAUCGCCUUCGCCCAGU |
| | Q74P, | ACCUGCAGCAGUGCCCUUUCGAGGACCACQUGAAGCUGGU |
| | N88D) ORF | GAACGAGGUGACCGAGUUCGCCAAGACCUGCGUGGCCGAC |
| | mRNA | GAGAGCGCCGAGAACUGCGACAAGAGCCUGCACACCCUGU |
| | sequence | UCGGCGACAAGCUGUGCACCGUGGCCACCCUGAGAGAAAC |
| | | UUACGGCGAGAUGGCCGACUGCUGCGCCAAGCAGGAGCCA |
| | | GAGCGGAACGAGUGCUUCCUCCAGCACAAGGACGACAACC |
| | | CUAACCUGCCUAGACUGGUAAGGCCUGAGGUGGACGUGAU |
| | | GUGUACCGCCUUCCACGACAACGAGGAGACAUUCCUGAAG |
| | | AAGUACCUGUACGAGAUCGCCAGAAGACACCCUUACUUCU |
| | | ACGCCCCUGAGUUGCUGUUCUUCGCGAAGAGAUACAAGGC |
| | | CGCCUUCACCGAGUGCUGCCAGGCCGCCGAUAAGGCCGCG |
| | | UGCCUGCUGCCUAAGCUGGACGAGCUGAGAGACGAGGGCA |
| | | AGGCAUCCAGCGCUAAGCAGAGACUGAAGUGCGCCAGCCU |
| | | GCAGAAGUUCGGAGAGAGAGCUUUCAAGGCGUGQGCAGU |
| | | GGCUAGAUUGAGCCAAAGAUUCCCUAAGGCAGAAUUCGCU |
| | | GAGGUGAGCAAGCUCGUGACUGACCUGACCAAGGUGCAUA |
| | | CAGAGUGCUGUCACGGCGACCUGCUGGAGUGCGCCGACGA |
| | | CAGAGCCGACCUGGCCAAGUACAUCUGCGAGAACCAGGAC |
| | | AGCAUCAGCAGCAACCUGAAGGAGUGUUGUGAGAAGQCU |
| | | CUAUUGGAGAAGAGUCACUGCAUUGCCGAGGUGGAGAAC |
| | | GACGAGAUGCCUGCGGAUCUGCCAAGCUUGGQGGCCGACU |
| | | UCGUGGAGAGCAAGGACGUGUGCAAGAACUACGCCGAGGC |
| | | CAAGCACGUUUUCCUGGGCAUGUUCCUCUACGAGUACQCA |
| | | CGCAGACAUCCAGACUACAGCGUGGUGCUGCUGCUGAGAC |
| | | UGGCUAAGACAUACGAAACUACCCUGGAGAAGUGCUGCGC |
| | | AGCGGCGGACCCUCACGAGUGUUACGCCAAGGUGUUCGAC |
| | | GAGUUCAAGCCUCUGGUGGAGGAGCCUCAGAACCUGAUCA |
| | | AGCAGAACUGUGAGCUGUUCGAGCAGCUCGGCGAGUACAA |
| | | GUUCCAGAACGCCCUGUUGGUCCQCUACACCAAGAAGGUG |
| | | CCUCAAGUAAGUACCCCUACCCUGGUAGAGGUUAGUAGAA |
| | | ACCUGGGCAAGGUGGGCAGCAAGUGCUQUAAGCACCCAGA |
| | | AGCUAAGAGGAUGCCUUGCGCCGAGGACUACCUGUCCGUU |
| | | GUGCUGAACCAQCUGUGCGUGCUGCACGAGAAGACCCCUG |
| | | UGAGCGACAGAGUGACAAAGUGUUGCACCGAGAGCUUAG |
| | | UGAAUAGAAGACCUUGCUUCAGCGCCCUGGAAGUUGACGA |
| | | AACUUACGUGCCUAAGGAGUUCAACGCCGAGACUUUCACA |
| | | UUCCACGCCGACAUUUGCACUCUGAGCGAGAAGGAGAGAC |
| | | AGAUCAAGAAGCAGACCGCCCUCGUAGAGUUGGUCAAGCA |
| | | CAAGCCGAAGGCAACCAAGGAACAGCUUAAGGCCGUGAUG |
| | | GACGACUUCGCAGCCUUCGUCGAGAAGUGUUGUAAGGCCG |
| | | ACGACAAGGAGACUUGCUUCGCAGAGGAAGGCAAGAAGU |
| | | UGGUAGCCGCCUCUCAGGCUGCCUUGGGACUCGGUGGCGG |
| | | CAGCGCCCCUACCAGCAGCUCCACAAAGAAGACGCAGCUG |
| | | CAGCUGGAGCACCUGCUACUAGACUDACAGAUGAUCCUGA |
| | | ACGGCAUCAACAACUACAAGAACCCGAAGCUCACCAGAAU |
| | | GCUGACAUUCAAGUUCUACAUGCCUAAGAAGGCCACUGAG |
| | | CUGAAGCACCUUCAGUGCCUGGAGGAGGAACUUAAGCCAC |
| | | UGGAAGAGGCCCUGAACCUGGCCCCUAGUAAGAAUUUCCA |
| | | CCUCAGACCUCGCGACCUUAUUAGCGACAUCAACGUGAUC |
| | | GUGCUCGAAUUAAAGGGCUCCGAGACAACAUUCAUGUGCG |
| | | AAUACGCCGACGAGACAGCCACCAUCGUGGAGUUCCUGAA |
| | | CCGCUGGAUCACCUUCUGCCAGAGCAUCAUCUCUACUCUG |
| | | ACC |
| 12 | Leader- | MKWVTFISLLFLESSAYSRGVFRRDAHKSEVAHRFKDLGEE |
| | HSA-(G3S)- | NFKALVLIAFAQYLQQCPFEDHVKLVNEVTEFAKTCVADESAE |
| | (Human | NCDKSLHTLFGDKLCTVATLRETYGEMADCCAKQEPERNECF |
| | IL-2, V9IK) | LQHKDDNPNLPRLVRPEVDVMCTAFHDNEETFLKKYLYEIAR |
| | | RHPYFYAPELLFFAKRYKAAFTECCQAADKAACLLPKLDELR |
| | | DEGKASSAKQRLKCASLQKFGERAFKAWAVARLSQRFPKAEF |
| | | AEVSKLVTDLTKVHTECCHGDLLECADDRADLAKYICENQDSI |
| | | SSKLKECCEKPLLEKSHCIAEVENDEMPADLPSLAADFVESKD |
| | | VCKNYAEAKDVFLGMFLYEYARRHPDYSVVLLLRLAKTYETT |
| | | LEKCCAAADPHECYAKVFDEFKPLVEEPQNLIKQNCELFEQLG |
| | | EYKFQNALLYRYTKKVPQVSTPTLVEVSRNLGKVGSKQCKHP |
| | | EAKRMPCAEDYLSVVLNQLCVLHEKTPVSDRVTKCCTESLVN |
| | | RRPCFSALEVDETYVPKEFNAETFTFHADICTLSEKERQIKKQT |
| | | ALVELVKHKPKATKEQLKAVMDDFAAFVEKCCKADDKETCF |
| | | AEEGKKLVAASQAALGLGGGSAPTSSSTKKTQLQLEHLLLDLQ |
| | | MILNGINNYKNPKLTRMLTFKFYMPKKATELKHLQCLEEELKP |
| | | LEEVLNLAQSKNEHLRPRDLISNINKIVLELKGSETTEMCEYAD |
| | | ETATIVEELNRWITECQSIISTLT |
| 13 | Leader- | MKWVTFISLLFLFSSAYSRGVFRRDAHKSEVAHRFKDLGEE |
| | HSA-(G3S)- | NFKALVLIAFAQYLQQCPFEDHVKLYNEVTEFAKTCVADESAE |
| | (Human | NCDKSLHTLFGDKLCTVATLRETYGEMADCCAKQEPERNECF |
| | IL-2 V69A, | LQHKDDNPNLPRLVRPEVDVMCTAFHDNEETFLKKYLYEIAR |
| | Q74P, | RHPYFYAPELLFFAKRYKAAFTECCQAADKAACLLPKLDELR |
| | V91K) | DEGKASSAKQRLKCASLQKFGERAFKAWAVARLSQRFPKAEF |
| | | AEVSKLVTDLTKVHTECCHGDLLECADDRADLAKYICENQDSI |
| | | SSKLKECCEKPLLEKSHCIAEVENDEMPADLPSLAADEVESKD |
| | | VCKNYAEAKDVFLGMFLYEYARRHPDYSVVLLLRLAKTYETT |
| | | LEKCCAAADPHECYAKVFDEFKPLVEEPQNLIKQNCELFEQLG |
| | | EYKFQNALLVRYTKKVPQVSTPTLVEVSRNLGKVGSKCCKHP |
| | | EAKRMPCAEDYLSVVLNQLCVLHBKTPVSDRVTKCCTESLVN |
| | | RRPCFSALEVDETYVPKEFNAETFTFHADICTLSEKERQIKKQT |
| | | ALVELVKHKPKATKEQLKAVMDDFAAFVEKCCKADDKETCF |
| | | AEEGKKLVAASQAALGLGGGSAPTSSSTKKTQLQLEHLLLDLQ |
| | | MILNGINNYKNPKLTRMLTEKEYMPKKATELKHLQCLEFELKP |
| | | LEBALNLAPSKNFHLRPRDLISNINKIVLELKGSETTEMCEYAD |
| | | ETATIVEFLNRWITFCQSIISTLT |

TABLE VI
Exemplary CTLA-4 binder sequences and IL-2 CTLA-4 sequences

| SEQ | | |
|---|---|---|
| ID | | |
| NO | Sequence information | Sequence |
| 17 | CTLA4_03 (Human, heavy | EVQLVQTGGGLSQFGESLRLSCAVSGFNVSNNYM |
| | chain only antibody binder | SWVRQAPGKGLEWVSIIYSGGGTHYADSVKGRFT |
| | to CTLA4, derived from | ISRDNSKNTLFLQMNSLRAEDTAVYYCARAVPVP |
| | HCAB mice) | HGTDIWGQGTMVTVSS |
| 18 | Leader-CTLA4.03- | MPLLLLLPLLWAGALAEVQLVQTGGGLSQFGESL |
| | (G4S)3-HSA-(G3S)- | RLSCAVSGFNVSNNYMSWVRQAPGKGLEWVSIIYSGG |
| | (Human IL-2)-V5 tag | GTHYADSVKGRFTISRDNSKNTLFLQMNSLRAEDTAV |
| | | YYCARAVPVPHGTDIWGQGTMVTVSSGGGGSGGGG |
| | | SGGGGSAHKSEVAHRFKDLGEENFKALVLIAFAQ |
| | | YLQQCPFEDHVKLVNEVTEFAKTCVADESAENCD |
| | | KSLHTLFGDKLCTVATLRETYGEMADCCAKQEPE |
| | | RNECFLQHKDDNPNLPRLVRPEVDVMCTAFHDN |
| | | EETFLKKYLYEIARRHPYFYAPELLFFAKRYKAAF |
| | | TECCQAADKAACLLPKLDELRDEGKASSAKQRLK |
| | | CASLQKFGERAFKAWAVARLSQRFPKAEFAEVSK |
| | | LVTDLTKVHTECCHGDLLECADDRADLAKYICEN |
| | | QDSISSKLKECCEKPLLEKSHCIAEVENDEMPADL |
| | | PSLAADFVESKDVCKNYAEAKDVFLGMFLYEYA |
| | | RRHPDYSVVLLLRLAKTYETTLEKCCAAADPHEC |
| | | YAKVFDEFKPLVEEPQNLIKQNCELFEQLGEYKFQ |
| | | NALLVRYTKKVPQVSTPTLVEVSRNLGKVGSKCC |
| | | KHPEAKRMPCAEDYLSVVLNQLCVLHEKTPVSD |
| | | RVTKCCTESLVNRRPCFSALEVDETYVPKEFNAET |
| | | FTFHADICTLSEKERQIKKQTALVELVKHKPKATK |
| | | EQLKAVMDDFAAFVEKCCKADDKETCFAEEGKK |
| | | LVAASQAALGLGGGSAPTSSSTKKTQLQLEHLLL |
| | | DLQMILNGINNYKNPKLTRMLTFKFYMPKKATEL |
| | | KHLQCLEEELKPLEEVLNLAQSKNFHLRPRDLISNI |
| | | NVIVLELKGSETTFMCEYADETATIVEFLNRWITF |
| | | CQSIISTLTGKPIPNPLLGLDST |
| 21 | Leader-CTLA4.03- | AUGCCUCUGUUACUGCUGCUCCCACUUCUGUGGGCC |
| | (G4S)3-HSA-(G3S)- | GGCGCUCUGGCUGAGGUGCAGCUUGUGCAGACCGGCGG |
| | (Human IL-2)-V5 tag | AGGUCUGUCACAGUUCGGCGAAUCCCUAAGGCUCUCUU |
| | | GCGCCGUGUCUGGUUUCAACGUGAGCAACAACUAUAUG |
| | | AGUUGGGUUCGCCAGGCUCCAGGCAAGGGUCUGGAGUG |
| | | GGUGUCCAUUAUCUAUUCCGGCGGUGGCACACAUUACG |
| | | CCGACUCAGUCAAGGGUAGAUUCACUAUCAGUAGAGAC |
| | | AACUCAAAGAACACUCUUUUCCUACAAAUGAACAGCUU |
| | | GCGGGCAGAGGAUACGGCCGUGUAUUAUUGUGCCCGAG |
| | | CUGUCCCAGUGCCUCACGGAACAGAUAUUUGGGGACAA |
| | | GGUACAAUGGUGACCGUCUCUUCCGGCGGAGGAGGCUC |
| | | UGGAGGCGGAGGAUCUGGCGGAGGCGGCAGCGCCCAUA |
| | | AGAGCGAGGUAGCCCAUAGAUUCAAGGACCUUGGCGAA |
| | | GAGAACUUCAAGGCCUUAGUUCUCAUCGCCUUCGCCCA |
| | | AUACUUGCAGCAGUGUCCAUUCGAAGAUCACGUUAAGC |
| | | UUGUGAACGAGGUGACCGAAUUCGCUAAGACGUGCGUG |
| | | GCCGACGAGUCCGCUGAGAAUUGUGAUAAGUCACUGCA |
| | | CACUUUGUUCGGCGACAAGCUGUGCACCGUCGCCACCC |
| | | UGCGCGAAACAUACGGCGAAAUGGCGGACUGUUGUGCU |
| | | AAGCAGGAGCCAGAACGGAACGAGUGUUUCCUGCAGCA |
| | | UAAGGACGACAAUCCAAACCUCCCUAGGUUGGUGCGCC |
| | | CAGAAGUGGACGUCAUGUGUACCGCUUUCCACGAUAAC |
| | | GAGGAGACAUUCCUUAAGAAGUAUCUGUACGAGAUUGC |
| | | ACGGCGGCACCCUUACUUCUACGCCCCUGAGCUCCUGUU |
| | | CUUCGCAAAGCGGUAUAAGGCUGCAUUCACCGAGUGUU |
| | | GCCAGGCGGCAGACAAGGCCGCCUGCUUGCUCCCUAAG |
| | | CUUGACGAACUUAGGGACGAGGGAAAGGCUAGUAGCGC |
| | | CAAGCAGAGACUCAAGUGUGCAUCGCUACAGAAGUUCG |
| | | GAGAGAGAGCUUUCAAGGCGUGGGCUGUCGCGCGCCUG |
| | | UCCCAGAGGUUCCCAAAGGCCGAAUUCGCCGAGGUGAG |
| | | CAAGUUAGUGACAGAUCUCACCAAGGUGCACACUGAGU |
| | | GUUGCCACGGCGACUUAUUGGAGUGUGCAGACGAUCGG |
| | | GCUGACUUGGCAAAGUACAUCUGUGAGAACCAGGACUC |
| | | AAUUUCCAGUAAGCUGAAGGAGUGUUGCGAGAAGCCAC |
| | | UCUUGGAGAAGUCCCAUUGUAUAGCCGAAGUGGAGAAC |
| | | GACGAGAUGCCGGCUGACCUGCCGUCACUGGCAGCUGA |
| | | UUUCGUAGAGUCGAAGGACGUGUGUAAGAACUACGCUG |
| | | AAGCUAAGGACGUAUUCCUGGGAAUGUUCCUUUACGAG |
| | | UACGCCCGACGGCAUCCAGACUACUCAGUAGUGCUGCU |
| | | UCUGAGACUGGCCAAGACUUACGAGACUACCCUGGAGA |
| | | AGUGUUGUGCAGCGGCCGAUCCACACGAGUGUUACGCC |
| | | AAGGUGUUCGACGAAUUCAAGCCUCUGGUGGAGGAGCC |
| | | GCAGAACCUGAUCAAGCAGAAUUGCGAACUGUUCGAGC |
| | | AGUUGGGAGAAUAUAAGUUCCAGAACGCACUGCUAGUG |
| | | CGGUAUACCAAGAAGGUCCCACAGGUAUCCACUCCGAC |
| | | ACUGGUGGAAGUGUCCAGGAACCUCGGCAAGGUGGGAA |
| | | GCAAGUGCUGUAAGCACCCGGAGGCUAAGCGGAUGCCG |
| | | UGCGCCGAAGACUACCUCAGCGUGGUCCUUAAUCAGCU |
| | | GUGCGUGCUGCACGAGAAGACCCCAGUAAGUGACCGGG |
| | | UUACGAAGUGCUGUACUGAAUCACUUGUGAACAGAAGA |
| | | CCUUGUUUCUCCGCCCUCGAGGUGGACGAAACGUACGU |
| | | UCCGAAGGAAUUCAACGCCGAAACAUUCACCUUCCACG |
| | | CUGACAUUUGUACGCUUUCCGAGAAGGAGAGACAAAUC |
| | | AAGAAGCAGACUGCCCUGGUGGAAUUAGUGAAGCACAA |
| | | GCCGAAGGCCACAAAGGAGCAGCUGAAGGCAGUUAUGG |
| | | ACGACUUCGCUGCAUUCGUAGAGAAGUGCUGCAAGGCA |
| | | GACGACAAGGAAACUUGCUUCGCAGAGGAGGGAAAGAA |
| | | GCUCGUUGCUGCGUCUCAGGCGGCUCUGGGACUGGGAG |
| | | GAGGAUCCGCUCCUACAAGCAGUUCCACAAAGAAGACC |
| | | CAGCUCCAGCUUGAACAUCUGCUUCUCGACCUGCAAAU |
| | | GAUCCUCAACGGUAUCAAUAAUUACAAGAAUCCUAAGU |
| | | UGACUCGCAUGCUGACUUUCAAGUUCUAUAUGCCAAAG |
| | | AAGGCCACUGAGUUGAAGCACCUGCAGUGCCUCGAGGA |
| | | AGAACUGAAGCCUCUGGAGGAAGUCUUAAACCUGGCAC |
| | | AAAGCAAGAACUUCCAUCUGAGGCCUAGGGACCUCAUU |
| | | UCCAAUAUCAACGUUAUUGUGCUGGAGCUCAAGGGUAG |
| | | CGAGACGACAUUCAUGUGCGAGUACGCAGACGAGACAG |
| | | CGACUAUCGUCGAGUUCCUAAAUAGGUGGAUAACGUUC |
| | | UGCCAGUCAAUCAUCUCCACUCUGACCGGCAAGCCGAU |
| | | CCCUAACCCGUUGCUUGGAUUGGAUUCUACA |
| 19 | Leader-CTLA4.03- | MPLLLLLPLLWAGALAEVQLVQTGGGLSQFGES |
| | (G4S)3-HSA-(G3S)- | LRLSCAVSGFNVSNNYMSWVRQAPGKGLEWVSIIYSG |
| | (Human IL-2 D20T)- | GGTHYADSVKGRFTISRDNSKNTLFLQMNSLRAEDT |
| | V5 tag | AVYYCARAVPVPHGTDIWGQGTMVTVSSGGGGSGG |
| | | GGSGGGGSAHKSEVAHRFKDLGEENFKALVLIAF |
| | | AQYLQQCPFEDHVKLVNEVTEFAKTCVADESAEN |
| | | CDKSLHTLFGDKLCTVATLRETYGEMADCCAKQ |
| | | EPERNECFLQHKDDNPNLPRLVRPEVDVMCTAFH |
| | | DNEETFLKKYLYEIARRHPYFYAPELLFFAKRYKA |
| | | AFTECCQAADKAACLLPKLDELRDEGKASSAKQR |
| | | LKCASLQKFGERAFKAWAVARLSQRFPKAEFAEV |
| | | SKLVTDLTKVHTECCHGDLLECADDRADLAKYIC |
| | | ENQDSISSKLKECCEKPLLEKSHCIAEVENDEMPA |
| | | DLPSLAADFVESKDVCKNYAEAKDVFLGMFLYE |
| | | YARRHPDYSVVLLLRLAKTYETTLEKCCAAADPH |
| | | ECYAKVFDEFKPLVEEPQNLIKQNCELFEQLGEYK |
| | | FQNALLVRYTKKVPQVSTPTLVEVSRNLGKVGSK |
| | | CCKHPEAKRMPCAEDYLSVVLNQLCVLHEKTPVS |
| | | DRVTKCCTESLVNRRPCFSALEVDETYVPKEFNA |
| | | ETFTFHADICTLSEKERQIKKQTALVELVKHKPKA |
| | | TKEQLKAVMDDFAAFVEKCCKADDKETCFAEEG |
| | | KKLVAASQAALGLGGGSAPTSSSTKKTQLQLEHL |
| | | LLTLQMILNGINNYKNPKLTRMLTFKFYMPKKAT |
| | | ELKHLQCLEEELKPLEEVLNLAQSKNFHLRPRDLI |
| | | SNINVIVLELKGSETTFMCEYADETATIVEFLNRWI |
| | | TFCQSIISTLTGKPIPNPLLGLDST |
| 22 | Leader-CTLA4.03- | AUGCCUCUGCUGCUCUUACUGCCGCUGCUUUGGGCA |
| | (G4S)3-HSA-(G3S)- | GGAGCUCUGGCCGAGGUCCAGCUAGUGCAGACAGGCGG |
| | (Human IL-2 D20T)- | UGGACUCAGCCAGUUCGGUGAGUCACUGCGUCUAUCCU |
| | V5 tag | GCGCGGUUAGUGGCUUCAACGUGUCCAACAAUUAUAUG |
| | | AGUUGGGUGAGACAAGCACCUGGCAAGGGCCUUGAGUG |
| | | GGUGUCCAUUAUAUAUAGCGGUGGCGGCACCCACUACG |
| | | CUGACUCGGUGAAGGGUCGAUUCACAAUUAGCAGAGAC |
| | | AAUUCCAAGAAUACUCUGUUCCUGCAGAUGAACUCUCU |
| | | CAGGGCGGAGGACACCGCCGUGUACUACUGUGCCCGUG |
| | | CGGUGCCGGUCCCUCACGGUACAGACAUCUGGGGCCAG |
| | | GGUACCAUGGUCACCGUGUCAUCCGGAGGAGGCGGUUC |
| | | UGGUGGAGGAGGAAGUGGAGGCGGCGGCAGUGCACAUA |
| | | AGAGCGAGGUGGCCCAUAGAUUCAAGGAUUUAGGAGAG |
| | | GAGAACUUCAAGGCACUGGUCUUGAUCGCCUUCGCACA |
| | | AUAUCUUCAGCAGUGUCCGUUCGAAGAUCACGUGAAGC |
| | | UCGUCAACGAGGUGACUGAGUUCGCCAAGACCUGCGUG |
| | | GCAGACGAGUCGGCUGAGAAUUGUGAUAAGAGUCUGCA |
| | | CACCCUCUUCGGAGAUAAGCUGUGUACCGUGGCCACUC |
| | | UGAGGGAGACAUACGGCGAGAUGGCUGACUGUUGUGCA |
| | | AAGCAGGAGCCAGAGAGGAACGAGUGUUUCUUGCAGCA |
| | | UAAGGACGACAACCCAAAUCUGCCUCGUCUGGUUAGAC |
| | | CUGAAGUGGACGUAAUGUGCACCGCUUUCCACGACAAC |
| | | GAGGAGACGUUCCUGAAGAAGUAUCUGUACGAGAUUGC |
| | | UAGAAGGCACCCAUACUUCUACGCACCAGAGCUGUUGU |
| | | UCUUCGCUAAGAGAUACAAGGCCGCUUUCACCGAGUGC |
| | | UGCCAAGCAGCAGACAAGGCUGCUUGCCUGCUCCCUAA |
| | | GCUGGACGAGCUGAGAGACGAGGGUAAGGCUUCAUCAG |
| | | CCAAGCAAAGAUUGAAGUGUGCAUCACUGCAGAAGUUC |
| | | GGCGAAAGAGCAUUCAAGGCUUGGGCCGUCGCCAGGCU |
| | | GUCUCAGAGGUUCCCGAAGGCCGAGUUCGCUGAAGUGU |
| | | CUAAGUUAGUGACCGAUCUAACAAAGGUGCACACAGAG |
| | | UGUUGCCACGGAGAUCUGCUUGAGUGCGCCGACGAUCG |
| | | AGCCGAUCUUGCAAAGUACAUCUGUGAGAACCAGGAUU |
| | | CCAUCUCCUCUAAGUUGAAGGAGUGCUGUGAGAAGCCA |
| | | CUUCUCGAGAAGUCUCACUGCAUCGCAGAAGUCGAGAA |
| | | CGACGAAAUGCCAGCUGACCUGCCGAGCUUGGCCGCAG |
| | | AUUUCGUCGAGAGUAAGGACGUUUGCAAGAAUUACGCU |
| | | GAGGCUAAGGACGUCUUCCUGGGCAUGUUCUUGUACGA |
| | | AUACGCCAGGAGGCACCCGGAUUACUCAGUGGUACUUU |
| | | UACUGCGACUGGCUAAGACCUACGAAACGACACUGGAG |
| | | AAGUGCUGCGCAGCCGCCGAUCCUCACGAGUGCUACGC |
| | | UAAGGUUUUCGACGAGUUCAAGCCGCUCGUGGAAGAAC |
| | | CUCAGAACUUAAUCAAGCAGAAUUGUGAGCUAUUCGAA |
| | | CAGCUCGGAGAGUAUAAGUUCCAGAACGCCCUCCUGGU |
| | | GCGGUACACAAAGAAGGUUCCACAGGUCAGCACCCCGA |
| | | CCCUCGUUGAGGUGAGCCGCAACCUCGGCAAGGUUGGC |
| | | UCUAAGUGUUGUAAGCAUCCGGAAGCGAAGCGCAUGCC |
| | | UUGUGCAGAAGAUUAUCUUUCAGUUGUGUUGAACCAGC |
| | | UCUGUGUCCUGCACGAGAAGACCCCAGUUUCCGACCGC |
| | | GUUACCAAGUGUUGCACAGAAAGCUUGGUCAACCGGAG |
| | | GCCUUGCUUCAGCGCAUUGGAGGUAGACGAAACCUACG |
| | | UCCCUAAGGAAUUCAACGCCGAAACAUUCACGUUCCAC |
| | | GCGGACAUUUGCACACUCUCCGAGAAGGAACGCCAGAU |
| | | UAAGAAGCAGACUGCCUUGGUUGAGCUGGUAAAGCACA |
| | | AGCCUAAGGCGACUAAGGAGCAGCUAAAGGCAGUUAUG |
| | | GACGACUUCGCUGCAUUCGUUGAGAAGUGCUGUAAGGC |
| | | CGACGAUAAGGAAACCUGCUUCGCCGAGGAAGGAAAGA |
| | | AGCUUGUUGCCGCUAGCCAGGCCGCACUGGGAUUGGGU |
| | | GGAGGCAGCGCACCAACUUCCUCUAGCACCAAGAAGAC |
| | | UCAAUUACAACUGGAACAUCUCUUACUAACACUCCAAA |
| | | UGAUACUGAACGGCAUAAACAACUAUAAGAACCCGAAG |
| | | UUGACCAGAAUGUUAACCUUCAAGUUCUACAUGCCUAA |
| | | GAAGGCUACGGAACUGAAGCACCUUCAGUGCCUAGAGG |
| | | AGGAGCUCAAGCCUCUCGAGGAGGUUCUGAACCUCGCA |
| | | CAGAGUAAGAAUUUCCACCUGCGCCCAAGGGACCUGAU |
| | | UUCCAAUAUUAACGUGAUCGUCCUUGAACUUAAGGGCA |
| | | GCGAAACCACUUUCAUGUGCGAAUACGCGGACGAAACU |
| | | GCUACCAUAGUUGAGUUCCUUAACCGCUGGAUCACAUU |
| | | CUGCCAGUCAAUCAUCUCUACAUUGACCGGAAAGCCGA |
| | | UUCCGAACCCACUCCUGGGACUUGAUAGCACA |
| 20 | Leader-CTLA4.03- | MPLLLLLPLLWAGALAEVQLVQTGGGLSQFGESL |
| | (G4S)3-HSA-(G3S)- | RLSCAVSGFNVSNNYMSWVRQAPGKGLEWVSIIYSGG |
| | (Human IL-2 N88D)- | GTHYADSVKGRFTISRDNSKNTLFLQMNSLRAEDTAV |
| | V5 tag | YYCARAVPVPHGTDIWGQGTMVTVSSGGGGSGGGG |
| | | SGGGGSAHKSEVAHRFKDLGEENFKALVLIAFAQ |
| | | YLQQCPFEDHVKLVNEVTEFAKTCVADESAENCD |
| | | KSLHTLFGDKLCTVATLRETYGEMADCCAKQEPE |
| | | RNECFLQHKDDNPNLPRLVRPEVDVMCTAFHDN |
| | | EETFLKKYLYEIARRHPYFYAPELLFFAKRYKAAF |
| | | TECCQAADKAACLLPKLDELRDEGKASSAKQRLK |
| | | CASLQKFGERAFKAWAVARLSQRFPKAEFAEVSK |
| | | LVTDLTKVHTECCHGDLLECADDRADLAKYICEN |
| | | QDSISSKLKECCEKPLLEKSHCIAEVENDEMPADL |
| | | PSLAADFVESKDVCKNYAEAKDVFLGMFLYEYA |
| | | RRHPDYSVVLLLRLAKTYETTLEKCCAAADPHEC |
| | | YAKVFDEFKPLVEEPQNLIKQNCELFEQLGEYKFQ |
| | | NALLVRYTKKVPQVSTPTLVEVSRNLGKVGSKCC |
| | | KHPEAKRMPCAEDYLSVVLNQLCVLHEKTPVSD |
| | | RVTKCCTESLVNRRPCFSALEVDETYVPKEFNAET |
| | | FTFHADICTLSEKERQIKKQTALVELVKHKPKATK |
| | | EQLKAVMDDFAAFVEKCCKADDKETCFAEEGKK |
| | | LVAASQAALGLGGGSAPTSSSTKKTQLQLEHLLL |
| | | DLQMILNGINNYKNPKLTRMLTFKFYMPKKATEL |
| | | KHLQCLEEELKPLEEVLNLAQSKNFHLRPRDLISDI |
| | | NVIVLELKGSETTFMCEYADETATIVEFLNRWITF |
| | | CQSIISTLTGKPIPNPLLGLDS |
| 23 | Leader-CTLA4.03- | AUGCCUCUGCUCCUGCUUCUUCCUCUGCUCUGGGCU |
| | (G4S)3-HSA-(G3S)- | GGCGCCCUAGCCGAGGUGCAGCUGGUGCAGACCGGCGG |
| | (Human IL-2 N88D)- | CGGCCUGAGCCAGUUCGGCGAGAGCCUGAGACUGAGCU |
| | V5 tag | GCGCCGUGAGCGGCUUCAACGUGAGCAACAACUACAUG |
| | | AGCUGGGUGAGACAGGCCCCUGGCAAGGGCCUGGAGUG |
| | | GGUGAGCAUCAUCUACAGUGGCGGAGGUACCCACUACG |
| | | CCGACAGCGUGAAGGGCAGAUUCACCAUCAGCAGAGAC |
| | | AACAGCAAGAACACCCUGUUCCUGCAGAUGAACAGCCU |
| | | CAGGGCCGAGGACACCGCCGUGUACUACUGCGCCAGAG |
| | | CCGUGCCUGUGCCUCACGGCACCGACAUCUGGGGCCAG |
| | | GGCACCAUGGUGACCGUGAGUAGCGGUGGAGGCGGAAG |
| | | CGGAGGCGGUGGCUCUGGAGGUGGCGGCAGCGCCCACA |
| | | AGAGCGAGGUGGCCCACAGAUUCAAGGACCUGGGCGAG |
| | | GAGAACUUCAAGGCCCUGGUGCUGAUCGCCUUCGCCCA |
| | | GUACCUGCAGCAGUGCCCUUUCGAGGACCACGUGAAGC |
| | | UGGUGAACGAGGUGACCGAGUUCGCCAAGACCUGCGUG |
| | | GCCGACGAGAGCGCCGAGAACUGCGACAAGAGCCUGCA |
| | | CACACUCUUCGGCGACAAGCUGUGCACCGUGGCCACCCU |
| | | GAGAGAGACUUACGGCGAGAUGGCCGACUGCUGCGCCA |
| | | AGCAGGAACCAGAGAGGAACGAGUGCUUCCUUCAGCAC |
| | | AAGGACGACAACCCUAACCUGCCUAGACUGGUUCGGCC |
| | | UGAGGUGGACGUGAUGUGCACUGCCUUCCACGACAACG |
| | | AGGAGACGUUCCUGAAGAAGUACCUGUACGAGAUCGCC |
| | | AGAAGACACCCUUACUUCUACGCCCCUGAGCUUCUGUU |
| | | CUUCGCAAAGAGAUACAAGGCCGCCUUCACCGAGUGCU |
| | | GCCAGGCCGCCGACAAGGCUGCCUGCCUCCUGCCAAAGC |
| | | UGGACGAGCUGAGAGACGAGGGCAAGGCCAGCUCUGCG |
| | | AAGCAGAGGCUGAAGUGCGCCAGCCUGCAGAAGUUCGG |
| | | AGAAAGAGCUUUCAAGGCCUGGGCCGUGGCCCGCUUGU |
| | | CUCAAAGAUUCCCUAAGGCGGAGUUCGCAGAAGUGAGC |
| | | AAGCUGGUCACCGACCUGACCAAGGUGCAUACUGAGUG |
| | | UUGCCACGGCGACCUGCUGGAGUGCGCCGACGACAGAG |
| | | CCGACCUGGCCAAGUACAUCUGCGAGAACCAGGACUCU |
| | | AUUAGUAGCAAGUUGAAGGAGUGUUGCGAGAAGCCGCU |
| | | ACUCGAGAAGAGUCACUGCAUUGCGGAGGUUGAGAACG |
| | | ACGAGAUGCCUGCCGACCUCCCAAGCCUGGCAGCCGAU |
| | | UUCGUGGAGAGCAAGGACGUGUGCAAGAAUUACGCUGA |
| | | GGCUAAGGACGUUUUCCUGGGCAUGUUCUUAUACGAAU |
| | | ACGCUAGACGGCACCCAGACUACAGCGUGGUCCUCCUCC |
| | | UGCGGCUUGCAAAGACGUACGAAACUACCCUGGAGAAG |
| | | UGCUGCGCGGCUGCUGACCCUCACGAGUGCUACGCCAA |
| | | GGUGUUCGACGAGUUCAAGCCUCUCGUGGAGGAGCCUC |
| | | AGAACCUGAUCAAGCAGAAUUGCGAGCUGUUCGAGCAG |
| | | UUAGGUGAGUACAAGUUCCAGAACGCCCUGCUUGUGCG |
| | | CUACACCAAGAAGGUGCCUCAGGUUUCCACCCCUACCCU |
| | | GGUAGAGGUGUCCAGAAACCUGGGCAAGGUGGGCAGCA |
| | | AGUGUUGCAAGCAUCCAGAAGCUAAGAGAAUGCCUUGC |
| | | GCCGAAGAUUACCUGUCUGUCGUGCUGAACCAGCUGUG |
| | | CGUGCUGCACGAGAAGACCCCUGUGAGCGACAGAGUGA |
| | | CCAAGUGCUGUACAGAAUCCCUAGUAAAUAGAAGACCU |
| | | UGCUUCUCCGCCCUAGAGGUGGACGAAACCUACGUGCC |
| | | UAAGGAGUUCAACGCCGAGACUUUCACGUUCCACGCGG |
| | | AUAUUUGUACACUCAGCGAGAAGGAGAGACAGAUCAAG |
| | | AAGCAGACCGCACUUGUGGAGCUCGUCAAGCACAAGCC |
| | | AAAGGCCACCAAGGAGCAACUCAAGGCCGUGAUGGACG |
| | | ACUUCGCAGCCUUCGUCGAGAAGUGUUGUAAGGCUGAC |
| | | GAUAAGGAAACCUGCUUCGCUGAGGAAGGCAAGAAGCU |
| | | UGUGGCCGCCAGUCAGGCUGCUCUUGGUCUUGGCGGCG |
| | | GAUCAGCACCUACCAGCAGCUCUACAAAGAAGACGCAG |
| | | CUGCAACUGGAGCACUUGCUGCUUGAUCUGCAAAUGAU |
| | | CCUGAACGGCAUCAACAAUUACAAGAACCCUAAGCUGA |
| | | CCAGAAUGCUGACCUUCAAGUUCUACAUGCCUAAGAAG |
| | | GCCACAGAGCUGAAGCAUUUACAGUGCCUGGAGGAGGA |
| | | GCUUAAGCCACUUGAAGAGGUGCUCAAUCUGGCCCAGU |
| | | CUAAGAACUUCCACCUCAGGCCUCGCGAUCUGAUUAGC |
| | | GACAUCAACGUAAUCGUGCUGGAACUUAAGGGAUCUGA |
| | | AACUACGUUCAUGUGCGAGUACGCCGACGAAACAGCCA |
| | | CCAUCGUGGAGUUCCUGAACCGAUGGAUCACCUUCUGC |
| | | CAGAGUAUUAUUUCAACCCUCACCGGCAAGCCUAUCCC |
| | | UAACCCACUGCUCGGACUCGACAGCACC |

TABLE VII
Exemplary IL-2 construct sequences

| mRNA | ORF Sequence | ORF Sequence | 5′ UTR | 3′ UTR | Construct |
|---|---|---|---|---|---|
| Name | (Amino Acid) | (Nucleotide) | Sequence | Sequence | Sequence |
| | SEQ ID NO | | | | |
| | 11 | 25 | 26 | 27 | 28 |
| HSA- | MKWVTFISLLFLFSSAYS | AUGAAGUGGGUGACC | GGGAAA | UGAUAA | SEQ ID |
| hsIL2.V69A. | RGVFRRDAHKSEVAHR | UUCAUCAGCCUGCUG | UAAGAG | UAGGCU | NO: 28 |
| Q74P.N88D | FKDLGEENFKALVLIAFA | UUCCUGUUCAGCAGC | AGAAAA | GGAGCC | consists |
| G5 | QYLQQCPFEDHVKLVN | GCCUACAGCAGAGGC | GAAGAG | UCGGUG | from 5′ to |
| Cap: C1 | EVTEFAKTCVADESAEN | GUGUUCAGAAGAGAC | UAAGAA | GCCUAGC | 3′ end: 5′ |
| Poly A tail: | CDKSLHTLFGDKLCTVA | GCCCACAAGAGCGAG | GAAAUA | UUCUUG | UTR of |
| 100 nt | TLRETYGEMADCCAKQ | GUGGCCCACAGAUUC | UAAGACC | CCCCUUG | SEQ |
| (SEQ ID NO: | EPERNECFLQHKDDNP | AAGGACCUGGGCGAG | CCGGCGC | GGCCUCC | ID |
| 29) | NLPRLVRPEVDVMCTA | GAGAACUUCAAGGCC | CGCCACC | CCCCAGC | NO: 26, |
| | FHDNEETFLKKYLYEIAR | CUGGUGCUGAUCGCC | | CCCUCCU | ORF |
| | RHPYFYAPELLFFAKRYK | UUCGCCCAGUACCUG | | CCCCUUC | sequence |
| | AAFTECCQAADKAACL | CAGCAGUGCCCUUUC | | CUGCACC | of SEQ |
| | LPKLDELRDEGKASSAK | GAGGACCACGUGAAG | | CGUACCC | ID NO: |
| | QRLKCASLQKFGERAFK | CUGGUGAACGAGGUG | | CCGUGG | 25, 3′ |
| | AWAVARLSQRFPKAEF | ACCGAGUUCGCCAAG | | UCUUUG | UTR of |
| | AEVSKLVTDLTKVHTEC | ACCUGCGUGGCCGAC | | AAUAAA | SEQ ID |
| | CHGDLLECADDRADLA | GAGAGCGCCGAGAAC | | GUCUGA | NO: 27 |
| | KYICENQDSISSKLKECC | UGCGACAAGAGCCUG | | GUGGGC | and Poly |
| | EKPLLEKSHCIAEVENDE | CACACCCUGUUCGGC | | GGC | A tail of |
| | MPADLPSLAADFVESK | GACAAGCUGUGCACC | | | SEQ ID |
| | DVCKNYAEAKDVFLGM | GUGGCCACCCUGAGA | | | NO: 29 |
| | FLYEYARRHPDYSVVLLL | GAAACUUACGGCGAG | | | |
| | RLAKTYETTLEKCCAAA | AUGGCCGACUGCUGC | | | |
| | DPHECYAKVFDEFKPLV | GCCAAGCAGGAGCCA | | | |
| | EEPQNLIKQNCELFEQL | GAGCGGAACGAGUGC | | | |
| | GEYKFQNALLVRYTKKV | UUCCUCCAGCACAAG | | | |
| | PQVSTPTLVEVSRNLGK | GACGACAACCCUAACC | | | |
| | VGSKCCKHPEAKRMPC | UGCCUAGACUGGUAA | | | |
| | AEDYLSVVLNQLCVLHE | GGCCUGAGGUGGACG | | | |
| | KTPVSDRVTKCCTESLV | UGAUGUGUACCGCCU | | | |
| | NRRPCFSALEVDETYVP | UCCACGACAACGAGG | | | |
| | KEFNAETFTFHADICTLS | AGACAUUCCUGAAGA | | | |
| | EKERQIKKQTALVELVK | AGUACCUGUACGAGA | | | |
| | HKPKATKEQLKAVMDD | UCGCCAGAAGACACC | | | |
| | FAAFVEKCCKADDKETC | CUUACUUCUACGCCC | | | |
| | FAEEGKKLVAASQAAL | CUGAGUUGCUGUUC | | | |
| | GLGGGSAPTSSSTKKTQ | UUCGCGAAGAGAUAC | | | |
| | LQLEHLLLDLQMILNGI | AAGGCCGCCUUCACC | | | |
| | NNYKNPKLTRMLTFKFY | GAGUGCUGCCAGGCC | | | |
| | MPKKATELKHLQCLEEE | GCCGAUAAGGCCGCG | | | |
| | LKPLEEALNLAPSKNFHL | UGCCUGCUGCCUAAG | | | |
| | RPRDLISDINVIVLELKG | CUGGACGAGCUGAGA | | | |
| | SETTFMCEYADETATIV | GACGAGGGCAAGGCA | | | |
| | EFLNRWITFCQSIISTLT | UCCAGCGCUAAGCAG | | | |
| | | AGACUGAAGUGCGCC | | | |
| | | AGCCUGCAGAAGUUC | | | |
| | | GGAGAGAGAGCUUUC | | | |
| | | AAGGCGUGGGCAGUG | | | |
| | | GCUAGAUUGAGCCAA | | | |
| | | AGAUUCCCUAAGGCA | | | |
| | | GAAUUCGCUGAGGUG | | | |
| | | AGCAAGCUCGUGACU | | | |
| | | GACCUGACCAAGGUG | | | |
| | | CAUACAGAGUGCUGU | | | |
| | | CACGGCGACCUGCUG | | | |
| | | GAGUGCGCCGACGAC | | | |
| | | AGAGCCGACCUGGCC | | | |
| | | AAGUACAUCUGCGAG | | | |
| | | AACCAGGACAGCAUC | | | |
| | | AGCAGCAAGCUGAAG | | | |
| | | GAGUGUUGUGAGAA | | | |
| | | GCCUCUAUUGGAGAA | | | |
| | | GAGUCACUGCAUUGC | | | |
| | | CGAGGUGGAGAACGA | | | |
| | | CGAGAUGCCUGCGGA | | | |
| | | UCUGCCAAGCUUGGC | | | |
| | | GGCCGACUUCGUGGA | | | |
| | | GAGCAAGGACGUGUG | | | |
| | | CAAGAACUACGCCGA | | | |
| | | GGCCAAGGACGUUUU | | | |
| | | CCUGGGCAUGUUCCU | | | |
| | | CUACGAGUACGCACG | | | |
| | | CAGACAUCCAGACUA | | | |
| | | CAGCGUGGUGCUGCU | | | |
| | | GCUGAGACUGGCUAA | | | |
| | | GACAUACGAAACUAC | | | |
| | | CCUGGAGAAGUGCUG | | | |
| | | CGCAGCGGCGGACCC | | | |
| | | UCACGAGUGUUACGC | | | |
| | | CAAGGUGUUCGACGA | | | |
| | | GUUCAAGCCUCUGGU | | | |
| | | GGAGGAGCCUCAGAA | | | |
| | | CCUGAUCAAGCAGAA | | | |
| | | CUGUGAGCUGUUCGA | | | |
| | | GCAGCUCGGCGAGUA | | | |
| | | CAAGUUCCAGAACGC | | | |
| | | CCUGUUGGUCCGCUA | | | |
| | | CACCAAGAAGGUGCC | | | |
| | | UCAAGUAAGUACCCC | | | |
| | | UACCCUGGUAGAGGU | | | |
| | | UAGUAGAAACCUGGG | | | |
| | | CAAGGUGGGCAGCAA | | | |
| | | GUGCUGUAAGCACCC | | | |
| | | AGAAGCUAAGAGGAU | | | |
| | | GCCUUGCGCCGAGGA | | | |
| | | CUACCUGUCCGUUGU | | | |
| | | GCUGAACCAGCUGUG | | | |
| | | CGUGCUGCACGAGAA | | | |
| | | GACCCCUGUGAGCGA | | | |
| | | CAGAGUGACAAAGUG | | | |
| | | UUGCACCGAGAGCUU | | | |
| | | AGUGAAUAGAAGACC | | | |
| | | UUGCUUCAGCGCCCU | | | |
| | | GGAAGUUGACGAAAC | | | |
| | | UUACGUGCCUAAGGA | | | |
| | | GUUCAACGCCGAGAC | | | |
| | | UUUCACAUUCCACGC | | | |
| | | CGACAUCUGUACUCU | | | |
| | | GAGCGAGAAGGAGAG | | | |
| | | ACAGAUCAAGAAGCA | | | |
| | | GACCGCCCUCGUAGA | | | |
| | | GUUGGUCAAGCACAA | | | |
| | | GCCGAAGGCAACCAA | | | |
| | | GGAACAGCUUAAGGC | | | |
| | | CGUGAUGGACGACUU | | | |
| | | CGCAGCCUUCGUCGA | | | |
| | | GAAGUGUUGUAAGG | | | |
| | | CCGACGACAAGGAGA | | | |
| | | CUUGCUUCGCAGAGG | | | |
| | | AAGGCAAGAAGUUGG | | | |
| | | UAGCCGCCUCUCAGG | | | |
| | | CUGCCUUGGGACUCG | | | |
| | | GUGGCGGCAGCGCCC | | | |
| | | CUACCAGCAGCUCCAC | | | |
| | | AAAGAAGACGCAGCU | | | |
| | | GCAGCUGGAGCACCU | | | |
| | | GCUACUAGACUUACA | | | |
| | | GAUGAUCCUGAACGG | | | |
| | | CAUCAACAACUACAA | | | |
| | | GAACCCGAAGCUCAC | | | |
| | | CAGAAUGCUGACAUU | | | |
| | | CAAGUUCUACAUGCC | | | |
| | | UAAGAAGGCCACUGA | | | |
| | | GCUGAAGCACCUUCA | | | |
| | | GUGCCUGGAGGAGGA | | | |
| | | ACUUAAGCCACUGGA | | | |
| | | AGAGGCCCUGAACCU | | | |
| | | GGCCCCUAGUAAGAA | | | |
| | | UUUCCACCUCAGACC | | | |
| | | UCGCGACCUUAUUAG | | | |
| | | CGACAUCAACGUGAU | | | |
| | | CGUGCUCGAAUUAAA | | | |
| | | GGGCUCCGAGACAAC | | | |
| | | AUUCAUGUGCGAAUA | | | |
| | | CGCCGACGAGACAGC | | | |
| | | CACCAUCGUGGAGUU | | | |
| | | CCUGAACCGCUGGAU | | | |
| | | CACCUUCUGCCAGAG | | | |
| | | CAUCAUCUCUACUCU | | | |
| | | GACC | | | |
| | SEQ ID NO | | | | |
| | 11 | 36 | 26 | 27 | 37 |
| HSA- | MKWVTFISLLFLFSSAYS | AUGAAGUGGGUGA | GGGAAA | UGAUAA | SEQ ID |
| hsIL2.V69A. | RGVFRRDAHKSEVAHR | CCUUCAUCAGCCUG | UAAGAG | UAGGCU | NO: 37 |
| Q74P.N88D | FKDLGEENFKALVLIAFA | CUGUUCCUGUUCA | AGAAAA | GGAGCC | consists |
| G5 | QYLQQCPFEDHVKLVN | GCAGCGCCUACAGC | GAAGAG | UCGGUG | from 5′ to |
| Cap: C1 | EVTEFAKTCVADESAEN | AGAGGCGUGUUCA | UAAGAA | GCCUAGC | 3′ end: 5′ |
| Poly A tail: | CDKSLHTLFGDKLCTVA | GAAGAGACGCCCAC | GAAAUA | UUCUUG | UTR of |
| 100 nt | TLRETYGEMADCCAKQ | AAGAGCGAGGUGG | UAAGACC | CCCCUUG | SEQ ID |
| (SEQ ID NO: | EPERNECFLQHKDDNP | CCCACAGAUUCAAG | CCGGCGC | GGCCUCC | NO: 26, |
| 29) | NLPRLVRPEVDVMCTA | GACCUGGGCGAGGA | CGCCACC | CCCCAGC | ORF |
| | FHDNEETFLKKYLYEIAR | GAACUUCAAGGCCC | | CCCUCCU | sequence |
| | RHPYFYAPELLFFAKRYK | UGGUGCUGAUCGC | | CCCCUUC | of SEQ |
| | AAFTECCQAADKAACL | CUUCGCCCAGUACC | | CUGCACC | ID NO: |
| | LPKLDELRDEGKASSAK | UGCAGCAGUGCCCU | | CGUACCC | 36, 3′ |
| | QRLKCASLQKFGERAFK | UUCGAGGACCACGU | | CCGUGG | UTR of |
| | AWAVARLSQRFPKAEF | GAAGCUGGUGAAC | | UCUUUG | SEQ ID |
| | AEVSKLVTDLTKVHTEC | GAGGUGACCGAGU | | AAUAAA | NO: 27 |
| | CHGDLLECADDRADLA | UCGCCAAGACCUGC | | GUCUGA | and Poly |
| | KYICENQDSISSKLKECC | GUGGCCGACGAGAG | | GUGGGC | A tail of |
| | EKPLLEKSHCIAEVENDE | CGCCGAGAACUGCG | | GGC | SEQ ID |
| | MPADLPSLAADFVESK | ACAAGAGCCUGCAC | | | NO: 29 |
| | DVCKNYAEAKDVFLGM | ACCCUGUUCGGCGA | | | |
| | FLYEYARRHPDYSVVLLL | CAAGCUGUGCACCG | | | |
| | RLAKTYETTLEKCCAAA | UGGCCACCCUGAGA | | | |
| | DPHECYAKVFDEFKPLV | GAAACUUACGGCGA | | | |
| | EEPQNLIKQNCELFEQL | GAUGGCCGACUGCU | | | |
| | GEYKFQNALLVRYTKKV | GCGCCAAGCAGGAG | | | |
| | PQVSTPTLVEVSRNLGK | CCAGAGCGGAACGA | | | |
| | VGSKCCKHPEAKRMPC | GUGCUUCCUCCAGC | | | |
| | AEDYLSVVLNQLCVLHE | ACAAGGACGACAAC | | | |
| | KTPVSDRVTKCCTESLV | CCUAACCUGCCUAG | | | |
| | NRRPCFSALEVDETYVP | ACUGGUAAGGCCU | | | |
| | KEFNAETFTFHADICTLS | GAGGUGGACGUGA | | | |
| | EKERQIKKQTALVELVK | UGUGUACCGCCUUC | | | |
| | HKPKATKEQLKAVMDD | CACGACAACGAGGA | | | |
| | FAAFVEKCCKADDKETC | GACAUUCCUGAAGA | | | |
| | FAEEGKKLVAASQAAL | AGUACCUGUACGAG | | | |
| | GLGGGSAPTSSSTKKTQ | AUCGCCAGAAGACA | | | |
| | LQLEHLLLDLQMILNGI | CCCUUACUUCUACG | | | |
| | NNYKNPKLTRMLTFKFY | CCCCUGAGUUGCUG | | | |
| | MPKKATELKHLQCLEEE | UUCUUCGCGAAGA | | | |
| | LKPLEEALNLAPSKNFHL | GAUACAAGGCCGCC | | | |
| | RPRDLISDINVIVLELKG | UUCACCGAGUGCUG | | | |
| | SETTFMCEYADETATIV | CCAGGCCGCCGAUA | | | |
| | EFLNRWITFCQSIISTLT | AGGCCGCGUGCCUG | | | |
| | | CUGCCUAAGCUGGA | | | |
| | | CGAGCUGAGAGACG | | | |
| | | AGGGCAAGGCAUCC | | | |
| | | AGCGCUAAGCAGAG | | | |
| | | ACUGAAGUGCGCCA | | | |
| | | GCCUGCAGAAGUUC | | | |
| | | GGAGAGAGAGCUU | | | |
| | | UCAAGGCGUGGGC | | | |
| | | AGUGGCUAGAUUG | | | |
| | | AGCCAAAGAUUCCC | | | |
| | | UAAGGCAGAAUUC | | | |
| | | GCUGAGGUGAGCA | | | |
| | | AGCUCGUGACUGAC | | | |
| | | CUGACCAAGGUGCA | | | |
| | | UACAGAGUGCUGU | | | |
| | | CACGGCGACCUGCU | | | |
| | | GGAGUGCGCCGACG | | | |
| | | ACAGAGCCGACCUG | | | |
| | | GCCAAGUACAUCUG | | | |
| | | CGAGAACCAGGACA | | | |
| | | GCAUCAGCAGCAAG | | | |
| | | CUGAAGGAGUGUU | | | |
| | | GUGAGAAGCCUCUA | | | |
| | | UUGGAGAAGAGUC | | | |
| | | ACUGCAUUGCCGAG | | | |
| | | GUGGAGAACGACGA | | | |
| | | GAUGCCUGCGGAUC | | | |
| | | UGCCAAGCUUGGCG | | | |
| | | GCCGACUUCGUGGA | | | |
| | | GAGCAAGGACGUG | | | |
| | | UGCAAGAACUACGC | | | |
| | | CGAGGCCAAGGACG | | | |
| | | UUUUCCUGGGCAU | | | |
| | | GUUCCUCUACGAGU | | | |
| | | ACGCACGCAGACAU | | | |
| | | CCAGACUACAGCGU | | | |
| | | GGUGCUGCUGCUG | | | |
| | | AGACUGGCUAAGAC | | | |
| | | AUACGAAACUACCC | | | |
| | | UGGAGAAGUGCUG | | | |
| | | CGCAGCGGCGGACC | | | |
| | | CUCACGAGUGUUAC | | | |
| | | GCCAAGGUGUUCG | | | |
| | | ACGAGUUCAAGCCU | | | |
| | | CUGGUGGAGGAGC | | | |
| | | CUCAGAACCUGAUC | | | |
| | | AAGCAGAACUGUGA | | | |
| | | GCUGUUCGAGCAGC | | | |
| | | UCGGCGAGUACAAG | | | |
| | | UUCCAGAACGCCCU | | | |
| | | GUUGGUCCGCUACA | | | |
| | | CCAAGAAGGUGCCU | | | |
| | | CAAGUAAGUACCCC | | | |
| | | UACCCUGGUAGAG | | | |
| | | GUUAGUAGAAACC | | | |
| | | UGGGCAAGGUGGG | | | |
| | | CAGCAAGUGCUGUA | | | |
| | | AGCACCCAGAAGCU | | | |
| | | AAGAGGAUGCCUU | | | |
| | | GCGCCGAGGACUAC | | | |
| | | CUGUCCGUUGUGC | | | |
| | | UGAACCAGCUGUGC | | | |
| | | GUGCUGCACGAGAA | | | |
| | | GACCCCUGUGAGCG | | | |
| | | ACAGAGUGACAAAG | | | |
| | | UGUUGCACCGAGA | | | |
| | | GCUUAGUGAAUAG | | | |
| | | AAGACCUUGCUUCA | | | |
| | | GCGCCCUGGAAGUU | | | |
| | | GACGAAACUUACGU | | | |
| | | GCCUAAGGAGUUCA | | | |
| | | ACGCCGAGACUUUC | | | |
| | | ACAUUCCACGCCGA | | | |
| | | CAUUUGCACUCUGA | | | |
| | | GCGAGAAGGAGAG | | | |
| | | ACAGAUCAAGAAGC | | | |
| | | AGACCGCCCUCGUA | | | |
| | | GAGUUGGUCAAGC | | | |
| | | ACAAGCCGAAGGCA | | | |
| | | ACCAAGGAACAGCU | | | |
| | | UAAGGCCGUGAUG | | | |
| | | GACGACUUCGCAGC | | | |
| | | CUUCGUCGAGAAG | | | |
| | | UGUUGUAAGGCCG | | | |
| | | ACGACAAGGAGACU | | | |
| | | UGCUUCGCAGAGG | | | |
| | | AAGGCAAGAAGUU | | | |
| | | GGUAGCCGCCUCUC | | | |
| | | AGGCUGCCUUGGG | | | |
| | | ACUCGGUGGCGGCA | | | |
| | | GCGCCCCUACCAGC | | | |
| | | AGCUCCACAAAGAA | | | |
| | | GACGCAGCUGCAGC | | | |
| | | UGGAGCACCUGCUA | | | |
| | | CUAGACUUACAGAU | | | |
| | | GAUCCUGAACGGCA | | | |
| | | UCAACAACUACAAG | | | |
| | | AACCCGAAGCUCAC | | | |
| | | CAGAAUGCUGACAU | | | |
| | | UCAAGUUCUACAUG | | | |
| | | CCUAAGAAGGCCAC | | | |
| | | UGAGCUGAAGCACC | | | |
| | | UUCAGUGCCUGGA | | | |
| | | GGAGGAACUUAAG | | | |
| | | CCACUGGAAGAGGC | | | |
| | | CCUGAACCUGGCCC | | | |
| | | CUAGUAAGAAUUU | | | |
| | | CCACCUCAGACCUC | | | |
| | | GCGACCUUAUUAGC | | | |
| | | GACAUCAACGUGAU | | | |
| | | CGUGCUCGAAUUAA | | | |
| | | AGGGCUCCGAGACA | | | |
| | | ACAUUCAUGUGCGA | | | |
| | | AUACGCCGACGAGA | | | |
| | | CAGCCACCAUCGUG | | | |
| | | GAGUUCCUGAACCG | | | |
| | | CUGGAUCACCUUCU | | | |
| | | GCCAGAGCAUCAUC | | | |
| | | UCUACUCUGACC | | | |

Note:
“G5” indicates that all uracils (U) in the mRNA are replaced by N1-methylpseudouracils.

### Stabilizing Elements

Naturally occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, the polynucleotide has an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Vims Capping Enzyme to generate the “Cap 0” structure: m7G(5′)ppp(5′)G (New England BioLabs, Ipswich, MA). Cap 1 structure may be generated using both Vaccinia Vims Capping Enzyme and a 2′-0 methyl-transferase to generate m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, the polynucleotide includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the polynucleotide (e.g., mRNA) includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, b-Galactosidase, EGFP), or a marker or selection protein (e.g. alpha-Globin, Galactokinase and Xanthine:guanine phosphoribosyl transferase (GPT)).

In some embodiments, the polynucleotide (e.g., mRNA) includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence. In some embodiments, an RNA (e.g., mRNA) does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

The polynucleotide (e.g., mRNA) may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, the polynucleotide (e.g., mRNA) has one or more AU-rich sequences removed. These sequences, sometimes referred to as AURES are destabilizing sequences found in the 3′UTR. The AURES may be removed from the RNA composition. Alternatively, the AURES may remain in the RNA composition.

### Signal Peptides

In some embodiments, the polynucleotide (e.g., mRNA) has an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and in prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane. A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid of the disclosure. In some embodiments, the signal peptide may comprise those described in WO 2021/154763, which is incorporated by reference in its entirety.

### Fusion Proteins

In some embodiments, the polynucleotide (e.g., mRNA) encodes an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the coronavirus antigen. Antigenic fusion proteins, in some embodiments, retain the functional property from each original protein.

### Scaffold Moieties

The polynucleotide (e.g., mRNA), in some embodiments, encodes fusion proteins that comprise coronavirus antigens linked to scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Fawson D. M. et al. Nature. 1991; 349:541-544). Ferritin self-assembles into nanoparticles with robust thermal and chemical stability. Thus, the ferritin nanoparticle is well suited to carry and expose antigens.

Fumazine synthase (FS) is also well suited as a nanoparticle platform for antigen display. FS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The FS monomer is 150 amino acids long, and consists of beta-sheets along with tandem alpha-helices flanking its sides. A number of different quaternary structures have been reported for FS, illustrating its morphological versatility: from homopentamers up to symmetrical assemblies of 12 pentamers forming capsids of 150 A diameter. Even FS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, the polynucleotide encodes a coronavirus antigen (e.g., SARS-CoV-2 S protein) fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, the polynucleotide (e.g., mRNA) encodes more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2 A peptides, has been described in the art (see for example, Kim, J. H. et al. (2011) PLoS ONE 6:el8556). In some embodiments, the linker is an F2A linker. In some embodiments, the fusion protein contains three domains with intervening linkers, having the structure: domain-linker-domain-linker-domain.

Cleavable linkers known in the art may be used in connection with the disclosure. Exemplary such linkers include F2A linkers, T2A linkers, P2A linkers, E2A linkers (See, e.g., WO2017/127750, which is incorporated herein by reference in its entirety). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding an antigen). In some embodiments, a codon optimized sequence shares less than 90% sequence identity to a naturally occurring or wild-type sequence (e.g., a naturally occurring or wild-type mRNA sequence encoding an antigen). In some embodiments, a codon optimized sequence shares less than 85% sequence identity to a naturally occurring or wild-type sequence (e.g., a naturally occurring or wild-type mRNA sequence encoding an antigen). In some embodiments, a codon optimized sequence shares less than 80% sequence identity to a naturally occurring or wild-type sequence (e.g., a naturally occurring or wild-type mRNA sequence encoding an antigen). In some embodiments, a codon optimized sequence shares less than 75% sequence identity to a naturally occurring or wild-type sequence (e.g., a naturally occurring or wild-type mRNA sequence encoding an antigen).

In some embodiments, a codon optimized sequence shares between 65% and 85% (e.g., between about 67% and about 85% or between about 67% and about 80%) sequence identity to a naturally occurring or wild-type sequence (e.g., a naturally occurring or wild-type mRNA sequence encoding a coronavirus antigen). In some embodiments, a codon optimized sequence shares between 65% and 75% or about 80% sequence identity to a naturally occurring or wild-type sequence (e.g., a naturally occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence. When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, the polynucleotide (e.g., mRNA) is not chemically modified and comprises the standard ribonucleotides consisting of adenosine, guanosine, cytosine and uridine. In some embodiments, nucleotides and nucleosides of the polynucleotide (e.g., mRNA) comprise standard nucleoside residues such as those present in transcribed RNA (e.g. A, G, C, or U). In some embodiments, nucleotides and nucleosides of the polynucleotide (e.g., mRNA) comprise standard deoxyribonucleosides such as those present in DNA (e.g. dA, dG, dC, or dT).

### Chemical Modifications

The polynucleotide (e.g., mRNA) comprises, in some embodiments, an RNA having an open reading frame encoding a coronavirus antigen, wherein the nucleic acid comprises nucleotides and/or nucleosides that can be standard (unmodified) or modified as is known in the art. In some embodiments, nucleotides and nucleosides of the polynucleotide (e.g., mRNA) comprise modified nucleotides or nucleosides. Such modified nucleotides and nucleosides can be naturally occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

The nucleic acids of the polynucleotide (e.g., mRNA) can comprise standard nucleotides and nucleosides, naturally-occurring nucleotides and nucleosides, non-naturally-occurring nucleotides and nucleosides, or any combination thereof.

Nucleic acids of the polynucleotide (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, the polynucleotide (e.g., mRNA) comprises uridine at one or more or all uridine positions of the nucleic acid. In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the polynucleotide (e.g., mRNA) may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C orA+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The polynucleotide (e.g., mRNA) may comprise one or more regions or parts that act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′ UTRs have features that play roles in translation initiation. They harbor signatures like Kozak sequences, which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTR also have been known to form secondary structures which are involved in elongation factor binding.

In some embodiments, the 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219, which are incorporated herein by reference in their entirety). CMV immediate-early 1 (IE1) gene (US2014/0206753, WO2013/185069, which are incorporated herein by reference in their entirety), the sequence GGGAUCCUACC (WO2014/144196) (SEQ ID NO: 89) may also be used. In another embodiment, 5′ UTR of a TOP gene is a 5′ UTR of a TOP gene lacking the 5′ TOP motif (the oligopyrimidine tract) (e.g., WO/2015/101414, WO2015/101415, WO/2015/062738, WO2015/024667, WO2015/024667; 5′ UTR element derived from ribosomal protein Large 32 (L32) gene (WO/2015/101414, WO2015/101415, WO/2015/062738), 5′ UTR element derived from the 5′ UTR of an hydroxysteroid (17-b) dehydrogenase 4 gene (HSD17B4) (WO2015/024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of the polynucleotide (e.g., mRNA). When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hours, 12 hours, 24 hours, 48 hours, and 7 days post-transfection.

3′ UTRs may be heterologous or synthetic.

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 2010/0293625 and PCT/US2014/069155, which are herein incorporated by reference in their entirety. It should be understood that any UTR from any gene may be incorporated into the regions of a nucleic acid. Furthermore, multiple wild-type UTRs of any known gene may be utilized. It is also within the scope of the present disclosure to provide artificial UTRs that are not variants of wild type regions. These UTRs or portions thereof may be placed in the same orientation as in the transcript from which they were selected or may be altered in orientation or location. Hence, a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 2010/0129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins that are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest that share at least one function, structure, feature, localization, origin, or expression pattern.

The untranslated region may also include translation enhancer elements (TEE). As a non-limiting example, the TEE may include those described in US Application No. 2009/0226470, herein incorporated by reference in its entirety, and those known in the art. In vitro Transcription of RNA cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA, which is then isolated and purified. In some embodiments, the DNA template includes an RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a composition of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the polynucleotide (e.g., mRNA) comprises 5′ terminal cap, for example, 7mG(5′)ppp(5′)NlmpNp.

### Chemical Synthesis

Solid-phase chemical synthesis. The polynucleotide (e.g., mRNA) may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of the polynucleotide (e.g., mRNA) by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase-catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the polynucleotide (e.g., mRNA) may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheo alveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC—HPLC), liquid chromatography-mass spectrometry (LCMS), capillary electrophoresis (CE) and capillary gel electrophoresis (CGE).

### Methods of Treatment

Provided herein are compositions (e.g., pharmaceutical compositions), methods, kits and reagents for prevention and/or treatment of cancer in humans and other mammals. The LNMP/RNA (e.g., mRNA or circRNA) formulations can be used as therapeutic or prophylactic agents. They may be used in medicine to prevent and/or treat cancer.

In one embodiment, the LNMP/RNA compositions (e.g., mRNA compositions or circRNA compositions) are used to provide prophylactic protection from cancer. Prophylactic protection from cancer can be achieved following administration of a LNMP/RNA composition (e.g., mRNA or circRNA composition such as vaccines). Vaccines can be administered once, twice, three times, four times or more but it is likely sufficient to administer the vaccine once (optionally followed by a single booster). It is more desirable, to administer the vaccine to an individual having cancer to achieve a therapeutic response. Dosing may need to be adjusted accordingly.

In some embodiments, the LNMP/RNA composition (e.g., mRNA or circRNA composition such as vaccines) is administered on a schedule for up to two months, up to three months, up to four month, up to five months, up to six months, up to seven months, up to eight months, up to nine months, up to ten months, up to eleven months, up to 1 year, up to 1 and % years, up to two years, up to three years, or up to four years. The schedule may be the same or varied. In some embodiments the schedule is weekly for the first 3 weeks and then monthly thereafter.

The LNMP/RNA composition (e.g., mRNA or circRNA composition such as vaccines) may be administered by any route. In some embodiments the composition is administered by an EVI or IV route.

At any point in the treatment, the patient may be examined to determine whether the mutations in the composition are still appropriate. Based on that analysis the composition may be adjusted or reconfigured to include one or more different mutations or to remove one or more mutations.

### Therapeutic and Prophylactic Compositions

Provided herein are compositions (e.g., pharmaceutical compositions), methods, kits and reagents for prevention, treatment or diagnosis of cancer in humans and other mammals,

In some embodiments, LNMP/RNA composition (e.g., mRNA or circRNA therapeutic composition such as vaccines) can be used in the priming of immune effector cells, for example, to activate peripheral blood mononuclear cells (PBMCs) ex vivo, which are then infused (re-infused) into a subject.

In exemplary embodiments, the LNMP/RNA composition (e.g., mRNA or circRNA therapeutic composition) can be administered to a subject (e.g., a mammalian subject, such as a human subject), and the RNA polynucleotides are translated in vivo to produce an antigenic polypeptide.

The LNMP/RNA composition (e.g., mRNA or circRNA therapeutic composition) may be induced for translation of a polypeptide {e.g., antigen or immunogen) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a composition containing a LNMP/RNA composition (e.g., mRNA or circRNA therapeutic composition) that contains a polynucleotide that has at least one a translatable region encoding an antigenic polypeptide.

An “effective amount” of a LNMP/RNA composition (e.g., mRNA or circRNA therapeutic composition) is provided based, at least in part, on the target tissue, target cell type, means of administration, physical characteristics of the polynucleotide (e.g., size, and extent of modified nucleosides) and other components of LNMP/RNA composition (e.g., mRNA or circRNA therapeutic composition), and other determinants. In general, an effective amount of LNMP/RNA composition (e.g., mRNA or circRNA therapeutic composition) provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the LNMP/RNA composition (e.g., mRNA or circRNA therapeutic composition), increased protein translation from the polynucleotide, decreased nucleic acid degradation (as demonstrated, for example, by increased duration of protein translation from a modified polynucleotide), or altered antigen specific immune response of the host cell.

In some embodiments, the LNMP/RNA composition (e.g., mRNA or circRNA therapeutic composition) may be used for treatment of cancer.

The LNMP/RNA composition (e.g., mRNA or circRNA therapeutic composition) may be administered prophylactically or therapeutically as part of an active immunization scheme to healthy individuals or early in cancer or during active cancer after onset of symptoms. In some embodiments, the amount of the LNMP/RNA composition (e.g., mRNA or circRNA therapeutic composition) provided to a cell, a tissue or a subject may be an amount effective for immune prophylaxis.

The LNMP/RNA composition (e.g., mRNA or circRNA therapeutic composition) may be administered with other prophylactic or therapeutic compounds. As a non-limiting example, a prophylactic or therapeutic compound may be an immune potentiator, adjuvant, or booster. As used herein, when referring to a composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In one embodiment, the LNMP/RNA composition (e.g., mRNA or circRNA therapeutic composition) may be administered intramuscularly or intradermally.

The LNMP/RNA composition (e.g., mRNA or circRNA therapeutic composition) may be utilized in various settings depending on the severity of the cancer or the degree or level of unmet medical need. As a non-limiting example, the LNMP/RNA composition (e.g., mRNA or circRNA therapeutic composition) may be utilized to treat any stage of cancer. The LNMP/mRNA or circRNA therapeutic composition) have superior properties in that they produce much larger antibody titers, T cell responses and produce responses early than commercially available anti-cancer vaccines. While not wishing to be bound by theory, the LNMP/RNA composition (e.g., mRNA or circRNA therapeutic composition), as mRNAs, have been better designed to produce the appropriate protein conformation on translation as the LNMP/RNA composition (e.g., mRNA or circRNA therapeutic composition) co-opt natural cellular machinery. Unlike traditional vaccines which are manufactured ex vivo and may trigger unwanted cellular responses, the LNMP/RNA composition (e.g., mRNA or circRNA therapeutic composition) are presented to the cellular system in a more native fashion.

A non-limiting list of cancers that the LNMP/RNA composition (e.g., mRNA or circRNA therapeutic composition) may treat is presented below. Peptide epitopes or antigens may be derived from any antigen of these cancers or tumors. Such epitopes are referred to as cancer or tumor antigens. Cancer cells may differentially express cell surface molecules during different phases of tumor progression. For example, a cancer cell may express a cell surface antigen in a benign state, yet down-regulate that particular cell surface antigen upon metastasis. As such, it is envisioned that the tumor or cancer antigen may encompass antigens produced during any stage of cancer progression. The methods of the invention may be adjusted to accommodate for these changes. For instance, several different LNMP/RNA compositions (e.g., mRNA or circRNA therapeutic compositions) may be generated for a particular patient. For instance, a first vaccine may be used at the start of the treatment. At a later time point, a new LNMP/RNA composition (e.g., mRNA or circRNA therapeutic composition) may be generated and administered to the patient to account for different antigens being expressed.

Provided herein are pharmaceutical compositions including the LNMP/RNA composition (e.g., mRNA or circRNA therapeutic composition) optionally in combination with one or more pharmaceutically acceptable excipients.

The LNMP/RNA compositions (e.g., mRNA or circRNA therapeutic composition) may be formulated or administered alone or in conjunction with one or more other components. For instance, the LNMP/RNA compositions (e.g., mRNA or circRNA therapeutic composition) may comprise other components including, but not limited to, immune potentiators (e.g., adjuvants). In some embodiments, the LNMP/RNA compositions (e.g., mRNA or circRNA therapeutic composition) do not include an immune potentiator or adjuvant (i.e., they are immune potentiator or adjuvant free).

In other embodiments, the LNMP/RNA compositions (e.g., mRNA or circRNA therapeutic composition) may be combined with any other therapy useful for treating the patient. For instance, a patient may be treated with t the LNMP/RNA compositions (e.g., mRNA or circRNA therapeutic composition) and an anti-cancer therapeutic agent. Thus, in one embodiment, the methods of the invention can be used in conjunction with one or more cancer therapeutics, for example, in conjunction with an anti-cancer therapeutic agent, a traditional cancer vaccine, chemotherapy, radiotherapy, etc. (e.g., simultaneously, or as part of an overall treatment procedure). Parameters of cancer treatment that may vary include, but are not limited to, dosages, timing of administration or duration or therapy; and the cancer treatment can vary in dosage, timing, or duration. Another treatment for cancer is surgery, which can be utilized either alone or in combination with any of the previous treatment methods. Any agent or therapy (e.g., traditional cancer vaccines, chemotherapies, radiation therapies, surgery, hormonal therapies, and/or biological therapies/immunotherapies) which is known to be useful, or which has been used or is currently being used for the prevention or treatment of cancer can be used in combination with a composition of the invention in accordance with the invention described herein. One of ordinary skill in the medical arts can determine an appropriate treatment for a subject.

Examples of such agents (i.e., anti-cancer therapeutic agents) include, but are not limited to, DNA-interactive agents including, but not limited to, the alkylating agents (e.g., nitrogen mustards, e.g. Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine); the DNA strand-breakage agents, e.g., Bleomycin; the intercalating topoisomerase II inhibitors, e.g., Intercalators, such as Amsacrine, Dactinomycin, Daunorubicin, Doxorubicin, Idarubicin, Mitoxantrone, and nonintercalators, such as Etoposide and Teniposide; the nonintercalating topoisomerase II inhibitors, e.g., Etoposide and Teniposde; and the DNA minor groove binder, e.g., Plicamydin; the antimetabolites including, but not limited to, folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea; tubulin Interactive agents including, but not limited to, colchicine, Vincristine and Vinblastine, both alkaloids and Paclitaxel and Cytoxan; hormonal agents including, but not limited to, estrogens, conjugated estrogens and Ethinyl Estradiol and Diethylstilbesterol, Chlortrianisen and Idenestrol; progestins such as Hydroxyprogesterone caproate, Medroxyprogesterone, and Megestrol; and androgens such as testosterone, testosterone propionate; fluoxymesterone, methyltestosterone; adrenal corticosteroid, e.g., Prednisone, Dexamethasone, Methylprednisolone, and Prednisolone; leutinizing hormone releasing hormone agents or gonadotropin-releasing hormone antagonists, e.g., leuprolide acetate and goserelin acetate; antihormonal antigens including, but not limited to, antiestrogenic agents such as Tamoxifen, antiandrogen agents such as Flutamide; and antiadrenal agents such as Mitotane and Aminoglutethimide; cytokines including, but not limited to, IL-1. alpha., IL-1β, IL-2, IL-3, IL-4, IL-5, IL-6, IL-7, IL-8, IL-9, IL-10, IL-11, IL-12, IL-13, IL-18, TGF-β, GM-CSF, M-CSF, G-CSF, TNF-a, TNF-β, LAF, TCGF, BCGF, TRF, BAF, BDG, MP, LIF, OSM, TMF, PDGF, IFN-a, IFN-β, IFN-γ, and Uteroglobins (U.S. Pat. No. 5,696,092); anti-angiogenics including, but not limited to, agents that inhibit VEGF (e.g., other neutralizing antibodies), soluble receptor constructs, tyrosine kinase inhibitors, antisense strategies, RNA aptamers and ribozymes against VEGF or VEGF receptors, Immunotoxins and coagulants, tumor vaccines, and antibodies.

Specific examples of anti-cancer therapeutic agents include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interieukin II, or rIL2), interferon alpha-2a; interferon alpha-2b; interferon alpha-nl; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; angiogenesis inhibitors; anti-dorsalizing morphogenetic protein-1; ara-CDP-DL-PTBA; BCR/ABL antagonists; CaRest M3; CARN 700; casein kinase inhibitors (ICOS); clotrimazole; collismycin A; collismycin B; combretastatin A4; crambescidin 816; cryptophycin 8; curacin A; dehydrodidemnin B; didemnin B; dihydro-5-azacytidine; dihydrotaxol, duocarmycin SA; kahalalide F; lamellarin-N triacetate; leuprolide+estrogen+progesterone; lissoclinamide 7; monophosphoryl lipid A+myobacterium cell wall sk; N-acetyldinaline; N-substituted benzamides; 06-benzylguanine; placetin A; placetin B; platinum complex; platinum compounds; platinum-triamine complex; rhenium Re 186 etidronate; RII retinamide; rubiginone B 1; SarCNU; sarcophytol A; sargramostim; senescence derived inhibitor 1; spicamycin D; tallimustine; 5-fluorouracil; thrombopoietin; thymotrinan; thyroid stimulating hormone; variolin B; thalidomide; velaresol; veramine; verdins; verteporfin; vinorelbine; vinxaltine; vitaxin; zanoterone; zeniplatin; and zilascorb.

The invention also encompasses administration of a composition comprising a LNMP/RNA composition (e.g., mRNA or circRNA therapeutic composition) in combination with radiation therapy comprising the use of x-rays, gamma rays and other sources of radiation to destroy the cancer cells. In some embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass.

In specific embodiments, an appropriate anti-cancer regimen is selected depending on the type of cancer. For instance, a patient with ovarian cancer may be administered a prophylactically or therapeutically effective amount of a composition comprising a LNMP/RNA composition (e.g., mRNA or circRNA therapeutic composition) in combination with a prophylactically or therapeutically effective amount of one or more other agents useful for ovarian cancer therapy, including but not limited to, intraperitoneal radiation therapy, such as P32 therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. Cancer therapies and their dosages, routes of administration and recommended usage are known in the art and have been described in such literature as the Physician's Desk Reference (56th ed., 2002).

In some embodiments, the LNMP/RNA compositions (e.g., mRNA or circRNA therapeutic compositions) are administered with a T cell activator such as be an immune checkpoint modulator. Immune checkpoint modulators include both stimulatory checkpoint molecules and inhibitory checkpoint molecules, i.e., an anti-CTLA4 and anti-PDI antibody. Stimulatory checkpoint inhibitors function by promoting the checkpoint process. Several stimulatory checkpoint molecules are members of the tumor necrosis factor (T F) receptor superfamily—CD27, CD40, OX40, GITR and CD 137, while others belong to the B7-CD28 superfamily—CD28 and ICOS. OX40 (CD134), is involved in the expansion of effector and memory T cells. Anti-OX40 monoclonal antibodies have been shown to be effective in treating advanced cancer. MED10562 is a humanized OX40 agonist. GITR, Glucocorticoid-Induced T FR family Related gene, is involved in T cell expansion Several antibodies to GITR have been shown to promote an anti-tumor responses. ICOS, Inducible T-cell costimulator, is important in T cell effector function. CD27 supports antigen-specific expansion of naive T cells and is involved in the generation of T and B cell memory. Several agonistic anti-CD27 antibodies are in development. CD122 is the Interleukin-2 receptor beta sub-unit. KTR-214 is a CD122-biased immune-stimulatory cytokine.

Inhibitory checkpoint molecules include but are not limited to PD-1, TEVI-3, VISTA, A2AR, B7-H3, B7-H4, BTLA, CTLA-4, IDO, KIR and LAG3, PDLI, PDL2, PDI, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDLI, PDL2, PDI, B7-H3, B7-H4, BTLA, HVEM, TIM3, GAL9, LAG3, VISTA, KIR, 2B4, CD 160, CGEN-15049, CHK 1, CHK2, A2aR, and B-7 family ligands. In some embodiments, the anti-PD-1 antibody is BMS-936558 (nivolumab). In other embodiments, the anti-CTLA-4 antibody is ipilimumab (trade name Yervoy, formerly known as MDX-010 and MDX-101).

The LNMP/RNA composition (e.g., mRNA or circRNA therapeutic composition) and anti-cancer therapeutic agent can be combined to enhance immune therapeutic responses even further. The LNMP/RNA composition (e.g., mRNA or circRNA therapeutic composition) and other therapeutic agents may be administered simultaneously or sequentially. When the other therapeutic agents are administered simultaneously, they can be administered in the same or separate formulations, but are administered at the same time. The other therapeutic agents are administered sequentially with one another and with the LNMP/RNA compositions (e.g., mRNA or circRNA therapeutic composition), when the administration of the other therapeutic agents and the LNMP/RNA composition (e.g., mRNA or circRNA therapeutic composition) is temporally separated. The separation in time between the administration of these compounds may be a matter of minutes or it may be longer, e.g. hours, days, weeks, months. For example, in some embodiments, the separation in time between the administration of these compounds is 1 hour, 2 hours, 3 hours 4 hours, 5 hours, 6 hours, 8 hours, 12 hours, 24 hours or more. In some embodiments, the separation in time between the administration of these compounds is 2 days, 3 days, 4 days, 5 days, 6 days, or 7 days or more. In some embodiments, the LNMP/RNA compositions (e.g., mRNA therapeutic composition) is administered before the anti-cancer therapeutic. In some embodiments, the LNMP/RNA composition (e.g., mRNA or circRNA therapeutic composition) is administered after the anti-cancer therapeutic.

Other therapeutic agents include but are not limited to anti-cancer therapeutic, adjuvants, cytokines, antibodies, antigens, etc.

The therapeutic agent may also include those that can treat and/or prevent chronic pain and/or symptoms of chronic pain, including but are not limited to:
(i) Opioid analgesics such as, morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, Buprenorphine, butorphanol, nalbuphine or pentazocine,
(ii) Non-steroidal anti-inflammatory drugs (NSAIDs) such as aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, Nabumetone, naproxen, oxaprozin, phenylbutazone, piroxicam, sulindac, tolmetin or zomepyrac, mobic (Meloxicam SR), or a pharmaceutically acceptable salt thereof
(iii) Barbiturate analgesics, such as amobarbital, aprobarbital, butabarbital, butalbital, methobarbital, metalbital, methohexital, pentobarbital, phenobarbital, secobarbital, talbutal, thiamaryl or thiopental or a pharmaceutically acceptable salt thereof,
(iv) benzodiazepines having analgesic activity, such as chlordiazepoxide, chlorazepic acid, diazepam, flurazepam, lorazepam, oxazepam, temazepam or triazolam or a pharmaceutically acceptable salt thereof;
(v) H1 antagonists having analgesic activity, for example, diphenhydramine, pyrilamine, promethazine, chlorpheniramine or chlorcyclidine or a pharmaceutically acceptable salt thereof;
(vi) Analgesics, for example, glutethimide, meprobamate, methacalone or dichloralphenazone or a pharmaceutically acceptable salt thereof;
(vii) Skeletal muscle relaxant, for example, baclofen, carisoprodol, chlorzoxazone, cyclobenzaprine, methocarbamol or orfrenazine or a pharmaceutically acceptable salt thereof;
(viii) NMDA receptor antagonist such as dextromethorphan ((+)-3-hydroxy-N-methylmorphinan) or its metabolite dextrorphan ((+)-3-hydroxy-N-methylmorphinan), Ketamine, memantine, pyrroloquinoline quinone or cis-4-(phosphonomethyl)-2-piperidine carboxylic acid, or a pharmaceutically acceptable salt thereof′
(ix) α-adrenergic agents such as doxazosin, tamsulosin, clonidine or 4-amino-6,7-dimethoxy-2-(5-methanesulfonamide-1,2,3,4-tetrahydroisoquinol-2-) Yl)-5-(2-pyridyl) quinazoline,
(x) tricyclic antidepressants such as desipramine, imipramine, amitriptyline or nortriptyline,
(xi) anticonvulsants such as carbamazepine or valproate,
(xii) tachykinin (NK) antagonists, in particular NK-3, NK-2 or NK-1 antagonists, for example (aR, 9R)-7-[3,5-bis (trifluoromethyl) benzyl]-8, 9, 10, 11-tetrahydro-9-methyl-5-(4-methylphenyl)-7H-[1,4] diazosino [2,1-g][1,7]naphtholidine-6-13-dione (TAK-637), 5-[[(2R, 3S)-2-[(1R)-1-[3,5-bis (trifluoromethyl) phenyl]ethoxy-3-(4-fluorophenyl)-4-) Morpholynyl]methyl]-1,2-dihydro-3H-1,2,4-triazol-3-one (MK-869), ranepitant, dapitant or 3-[[2-methoxy-5-(trifluoromethoxy) phenyl] Methyl Amy]-2-phenyl-piperidine (2S, 3S),
(xiii) muscarinic antagonists, for example oxybutin, tolterodine, propiverine, trospium chloride or darifenacin,
(xiv) COX-2 inhibitors, e.g. celecoxib, rofecoxib or valdecoxib,
(xv) non-selective COX inhibitors, preferably those with GI protection, such as nitroflurbiprofen (HCT-1026),
(xvi) coal tar analgesics, especially paracetamol,
(xvii) neuroleptics, such as droperidol,
(xviii) vanilloid receptor agonists (for example resiniferatoxin) or antagonists (for example capsazepine),
(xix) beta-adrenergic agents, such as propranolol,
(xx) a local anesthetic such as mexiletine,
(xxi) corticosteroids, e.g. dexamethasone,
(xxii) serotonin receptor agonists or antagonists,
(xxiii) cholinergic (nicotinic) analgesics,
(xxiv) Tramadol™,
(xxv) PDEV inhibitors such as sildenafil, vardenafil or tadalafil,

In some embodiments, provided methods include administering a LNMP/RNA composition (e.g., mRNA or circRNA therapeutic composition) in combination with an immune checkpoint modulator. In some embodiments, an immune checkpoint modulator, e.g., checkpoint inhibitor such as an anti-PD-1 antibody, is administered at a dosage level sufficient to deliver 100-300 mg to the subject. In some embodiments, an immune checkpoint modulator, e.g., checkpoint inhibitor such as an anti-PD-1 antibody, is administered at a dosage level sufficient to deliver 200 mg to the subject. In some embodiments, an immune checkpoint modulator, e.g., checkpoint inhibitor such as an anti-PD-1 antibody, is administered by intravenous infusion. In some embodiments, the immune checkpoint modulator is administered to the subject twice, three times, four times or more. In some embodiments, the immune checkpoint modulator is administered to the subject on the same day as the LNMP/RNA composition (e.g., mRNA or circRNA therapeutic composition) administration.

The LNMP/RNA composition (e.g., mRNA or circRNA therapeutic composition) may be formulated or administered in combination with one or more pharmaceutically-acceptable excipients. In some embodiments, the LNMP/RNA composition (e.g., mRNA or circRNA therapeutic composition) comprises at least one additional active substances, such as, for example, a therapeutically-active substance, a prophylactically-active substance, or a combination of both. The LNMP/RNA composition (e.g., mRNA or circRNA therapeutic composition) may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, the LNMP/RNA compositions (e.g., mRNA or circRNA therapeutic compositions) are administered to humans, human patients or subjects. The phrase “active ingredient” generally refers to the LNMP/RNA composition (e.g., mRNA or circRNA therapeutic composition) or the polynucleotides contained therein, for example, RNA polynucleotides (e.g., mRNA polynucleotides or circRNA polyribonucleotides) encoding antigenic polypeptides.

Formulations of the LNMP/RNA composition (e.g., mRNA or circRNA therapeutic composition) described herein may be prepared by any method known or hereafter developed in the art of pharmacology. In general, such preparatory methods include the step of bringing the active ingredient (e.g., mRNA polynucleotide or circRNA polyribonucleotide) into association with an excipient and/or one or more other accessory ingredients, and then, if necessary and/or desirable, dividing, shaping and/or packaging the product into a desired single- or multi-dose unit.

The LNMP/RNA composition (e.g., mRNA or circRNA therapeutic composition) can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with cancer RNA vaccines (e.g., for transplantation into a subject), hyaluronidase, nanoparticle mimics and combinations thereof.

### Kits

The present invention also provides a kit including a container having an RNA composition (e.g., mRNA or circRNA therapeutic composition) described herein. The kit may further include instructional material for applying or delivering the mRNA or circRNA therapeutic composition to a subject in accordance with a method of the present invention. The skilled artisan will appreciate that the instructions for applying the RNA composition (e.g., mRNA or circRNA therapeutic composition) in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD). The therapeutic composition (e.g., the mRNA or circRNA therapeutic composition) may include material for a single administration (e.g., single dosage form), or may include material for multiple administrations (e.g., a “multidose” kit).

The informational material of the kits is not limited in its form. In one embodiment, the informational material may include information about production of the therapeutic composition (e.g., the mRNA or circRNA therapeutic composition) described herein, the drug substance, or the drug product, molecular weight of the composition, the drug substance, or the drug product, concentration, date of expiration, batch or production site information, and so forth. In one embodiment, the informational material relates to methods for administering a dosage form of the mRNA or circRNA therapeutic composition. In one embodiment, the informational material relates to methods for administering a dosage form of the circRNA therapeutic composition.

In addition to a dosage form of the mRNA or circRNA therapeutic composition described herein, the kit may include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients may be included in the kit, but in different compositions or containers than a mRNA or circRNA therapeutic composition described herein. In such embodiments, the kit may include instructions for admixing a therapeutic composition (e.g., mRNA or circRNA therapeutic composition) or nucleic acid molecule (e.g., a mRNA or a circRNA) described herein and the other ingredients, or for using a therapeutic composition (e.g., mRNA or circRNA therapeutic composition) or nucleic acid molecule (e.g., a mRNA or circRNA) described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form of a therapeutic composition (e.g., mRNA or circRNA therapeutic composition) or nucleic acid molecule (e.g., a mRNA or circRNA) described herein may be provided in any form, e.g., liquid, dried or lyophilized form. It is preferred that a therapeutic composition (e.g., mRNA or circRNA therapeutic composition) or nucleic acid molecule (e.g., a mRNA or circRNA) described herein be substantially pure and/or sterile. When a therapeutic composition (e.g., mRNA or circRNA therapeutic composition) or nucleic acid molecule (e.g., a mRNA or circRNA) described herein is provided in a liquid solution, the liquid solution preferably is an aqueous solution, with a sterile aqueous solution being preferred. When a therapeutic composition (e.g., mRNA or circRNA therapeutic composition) or nucleic acid molecule (e.g., a mRNA or circRNA) described herein is provided as a dried form, reconstitution generally is by the addition of a suitable solvent. The solvent, e.g., sterile water or buffer, can optionally be provided in the kit.

The kit may include one or more containers for the therapeutic composition containing a dosage form described herein. In some embodiments, the kit contains separate containers, dividers or compartments for the composition and informational material. For example, the therapeutic composition or nucleic acid molecule may be contained in a bottle, vial, or syringe, and the informational material may be contained in a plastic sleeve or packet. In other embodiments, the separate elements of the kit are contained within a single, undivided container. For example, the dosage form of a therapeutic composition (e.g., mRNA or circRNA therapeutic composition) or nucleic acid molecule (e.g., a mRNA or circRNA) described herein is contained in a bottle, vial or syringe that has attached thereto the informational material in the form of a label. In some embodiments, the kit includes a plurality (e.g., a pack) of individual containers, each containing one or more unit dosage forms of a therapeutic composition (e.g., mRNA or circRNA therapeutic composition) or nucleic acid molecule (e.g., mRNA or circRNA) described herein. For example, the kit includes a plurality of syringes, ampules, foil packets, or blister packs, each containing a single unit dose of a dosage form described herein.

The containers of the kits can be airtight, waterproof (e.g., impermeable to changes in moisture or evaporation), and/or light-tight.

The kit optionally includes a device suitable for use of the dosage form, e.g., a syringe, pipette, forceps, measured spoon, swab (e.g., a cotton swab or wooden swab), or any such device.

The kits of the invention may include dosage forms of varying strengths to provide a subject with doses suitable for one or more of the initiation phase regimens, induction phase regimens, or maintenance phase regimens described herein. Alternatively, the kit may include a scored tablet to allow the user to administered divided doses, as needed.

### EXAMPLES

The invention now being generally described, it will be more readily understood by reference to the following examples, which are included merely for purposes of illustration of certain aspects and embodiments of the present invention, and are not intended to limit the invention.

### Example 1: Preparation and Modification of LNMP, and Formulation LNMP with mRNAs

This example describes the preparation of a structural component comprising natural lipids (e.g., lipids extracted from a natural source), and the modification of the structural component to form a lipid reconstructed natural messenger pack (LNMP). An example of an LNMP is an LPMP (reconstructed plant messenger pack). The preparation of natural messenger packs (NMPs), modification of NMP to prepare lipid reconstructed natural messenger packs (LNMP), and formulation of NMP and LNMP with mRNAs are illustrated by the preparation of PMP and preparation and formulation of LPMP, which may be accomplished utilizing the methods disclosed in International Patent Application Publication No. WO 2021/041301, which is incorporated herein by reference in its entirety.

In particular, all the experimental protocols disclosed in Examples 1-17 of International Patent Application Publication No. WO 2021/041301, are incorporated herein by reference in their entirety, including: Example 1. Isolation of Plant Messenger Packs from plants; Example 2. Production of purified Plant Messenger Packs (PMPs); Example 3. Plant Messenger Pack characterization; Example 4. Characterization of Plant Messenger Pack stability; Example 5. Loading PMPs with cargo; Example 6. Increasing PMP cellular uptake by formulation of PMPs with ionic liquids; Example 7. Modification of PMPs using ionizable lipids; Example 8. Formulation of LPMPs with microfluidics; Example 9. mRNA loading and delivery into lipid-reconstructed PMPs using ionizable lipids; Example 10. Cellular uptake of natural and reconstructed PMPs, with and without ionizable lipid modifications; Example 11. Increasing PMP cellular uptake by formulation of PMPs with cationic lipids; Example 12. Modification of PMPs using cationic lipids; Example 13. mRNA loading and delivery into lipid-reconstructed PMPs using cationic lipids; Example 14. Cellular uptake of natural and reconstructed PMPs, with and without cationic lipid modifications; Example 15. Improved loading using the cationic lipids GL67 and Ethyl PC; Example 16. Optimization of lipid ratios for mRNA loading; and Example 17. Optimization of lipid ratios for plasmid loading.

### Example 2: Preparation of an mRNA Therapeutic Composition

### Manufacture and Characterization of Polynucleotides

The manufacture of polynucleotides and/or parts or regions thereof may be accomplished utilizing the methods taught in International Patent Application Publication No. WO 2014/152027, which is incorporated herein by reference in its entirety. Purification methods may include those taught in International Patent Application Publication Nos. WO2014/152030 and WO2014/152031, which are incorporated herein by reference in their entirety. Detection and characterization methods of the polynucleotides may be performed as taught in International Patent Application Publication No. WO2014/144039, which is incorporated herein by reference in its entirety. Characterization of the polynucleotides may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Patent Application Publication Nos. WO2014/144711 and WO2014/144767, which are incorporated herein by reference in their entirety.

Additional details of the mRNA design and modifications may be found in WO 2021/154763 and WO 2021/188969, which are incorporated herein by reference in their entirety.

Formulation of LPMP with an mRNA

### General Protocols and Experimental Designs

The protocols and detailed experimental procedures of the preparation of natural messenger packs (NMP), and modification of NMP to prepare lipid reconstructed natural messenger packs (LNMP), and formulations of NMP and LNMP with mRNAs have been discussed in Example 1, which lists all the experimental protocols disclosed in Examples 1-17 of International Patent Application Publication No. WO 2021/041301, all of which are incorporated herein by reference in their entirety. Additionally, the protocols and detailed experimental procedures of the preparation and formulations of LNMP (with or without mRNA) where the natural lipids in the LNMP is extracted from a bacteria source have been discussed in International Application No. PCT/US22/50571, which is incorporated herein by reference in its entirety. These protocols and detailed experimental procedures were followed when preparing NMP (e.g., with bacteria lipids), LNMP (e.g., with bacteria lipids), PMP, LPMP, LNP, and NMP (e.g., with bacteria lipids), LNMP (e.g., with bacteria lipids), PMP, LPMP, or LNP formulated with mRNAs in this example.

In this example, LPMP was used as an example of an LNMP. Briefly, the isolation and purification of crude plant messenger packs (PMPs) from lemon, and characterization of these PMPs followed the experimental designs and protocols for plants sources described in Example 1: Isolation of Plant Messenger Packs from plants; Example 2: Production of purified Plant Messenger Packs (PMPs); Example 3: Plant Messenger Pack characterization; and Example 4: Characterization of Plant Messenger Pack stability, International Patent Application Publication No. WO 2021/041301, which is incorporated herein by reference in its entirety.

The modifications of natural PMP or reconstructed lemon or algae LPMPs with cholesterol and PEG-lipid followed the experimental designs and protocols for plants sources described in Example 6: Increasing PMP cellular uptake by formulation of PMPs with ionic liquids; Example 7: Modification of PMPs using ionizable lipids; Example 10: Cellular uptake of natural and reconstructed PMPs, with and without ionizable lipid modifications; Example 11: Increasing PMP cellular uptake by formulation of PMPs with cationic lipids; Example 12: Modification of PMPs using cationic lipids; and Example 14: Cellular uptake of natural and reconstructed PMPs, with and without cationic lipid modifications, International Patent Application Publication No. WO 2021/041301, which is incorporated herein by reference in its entirety.

Formulations of the PMPs and lemon-lipid- or algae-lipid-reconstructed LPMPs with mRNAs followed the experimental designs and protocols for nucleic acids loading described in Example 5: Loading PMPs with cargo; Example 8: Formulation of LPMPs with microfluidics; Example 9: mRNA loading and delivery into lipid-reconstructed PMPs using ionizable lipids; Example 13: mRNA loading and delivery into lipid-reconstructed PMPs using cationic lipids; Example 15: Improved loading using the cationic lipids GL67 and Ethyl PC; Example 16: Optimization of lipid ratios for mRNA loading; and Example 17: Optimization of lipid ratios for plasmid loading, International Patent Application Publication No. WO 2021/041301, which is incorporated herein by reference in its entirety.

### Formulations of C12-200 LPMP/mRNA

This example describes the formulation of LPMP formulated with C12-200 as the ionizable lipid, sterols, and PEG lipids, which encapsulates an mRNA (e.g., 2:1 eGFP:EPO).

LNP formulations. A LNP (lipid nanoparticle) formulation, as control, was prepared to result in ionizable lipid:structural lipid:sterol:PEG-lipid (C12-200:DOPE:cholesterol:DMPE-PEG2k) at a molar ratio of 35:16:46.5:2.5, respectively, as shown in Table 2. To prepare this formulation, the above lipids were solubilized in ethanol, mixed at the above molar ratios, and diluted in ethanol (organic phase) to obtain total lipid concentration of 12.5 mM total lipid concentration. An mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 15:1.

C12-200 LPMP formulations. A C12-200 recLemon LPMP formulation was prepared to result in ionizable lipid:natural lipids:sterol:PEG-lipid (C12-200:lemon lipid:cholesterol:DMPE-PEG2k) at a molar ratio of 35:50:12.5:2.5, respectively, as shown in Table 2. To prepare this formulation, the above lipids were solubilized in ethanol, except for lemon lipid, which was solubilized in 4:1 DMF:methanol. The lipids were then mixed at the above molar ratios, and diluted to obtain total lipid concentration of 5.5 mM. An mRNA solution (aqueous phase) was prepared with RNAse-free water and 100 mM citrate buffer pH 3 for a final concentration of 50 mM citrate buffer.

The lipid mixture and mRNA solution were mixed at a 1:3 ratio by volume, respectively, on the NanoAssemblr Ignite (Precision Nanosystems) at a total flow rate of 9 mL/min. The resulting formulations were then loaded into Slide-A-Lyzer G2 dialysis cassettes (10 k MWCO) and dialyzed in 200 times sample volume of 1×PBS for 2 hours at room temperature with gentle stirring. The PBS solution was refreshed, and the formulations were further dialyzed for at least 14 hours at 4° C. with gentle stirring. The dialyzed formulations were then collected and concentrated by tangential flow filtration (TFF) using Sartorius VIVAFLOW® 50R cassettes (100 k MWCO) at fixed feed flowrate of 150 ml/minute. The TFF-concentrated formulations were then further by centrifugation at 3000×g (Amicon Ultra centrifugation filters, 100 k MWCO). The concentrated particles were characterized for size, polydispersity, and particle concentration using Zetasizer Ultra (Malvern Panalytical). The mRNA encapsulation efficiency was characterized by Quant-iT RiboGreen RNA Assay Kit (ThermoFisher Scientific). The particles were diluted to the desired mRNA concentration to get a final 10% sucrose solution in PBS. The formulations were then flash frozen in liquid nitrogen.

The resulting formulations with mRNA are shown in Table 2.

TABLE 2
The characterizations of a typical LPMP formulation with mRNA

| | Ionizable: | | | | | | | | | |
|---|---|---|---|---|---|---|---|---|---|---|
| | Structural: | | | | | | | | | |
| | Sterol: | | | | | | | | | Encapsulation |
| Form- | PEG-lipid | | | | PEG- | | N: | Size | | efficiency |
| ulation | Molar ratio | Ionizable | Structural | Sterol | lipid | Cargo | P | (nm) | PDI | (%) |
| LNP | 35:16: | C12-200 | DOPE | Chol | DMPE- | an | 15:1 | 78.47 | 0.126 | 94.4 |
| | 46.5:2.5 | | | | PEG2k | mRNA | | | | |
| LPMP/ | 35:50: | C12-200 | Naturally | Chol | DMPE- | an | | 96.49 | 0.300 | 94.8 |
| mRNA | 12.5:2.5 | | derived | | PEG2k | mRNA | | | | |
| (recLemon) | | | lemon lipid | | | | | | | |

Additional mRNA Design for Loading into LPMP/mRNA Formulation

Exemplary sequences of payload are shown in Table 3.

TABLE 3

| SEQ ID | | |
|---|---|---|
| NO: | Payload | Sequence |
| 59 | tdTomato | ATGGTGAGCAAGGGCGAGGAGGTCATCAAAGAGTTCATGCGCTTCAAGGTGC |
| | Cytosolic | GCATGGAGGGCTCCATGAACGGCCACGAGTTCGAGATCGAGGGCGAGGGCG |
| | | AGGGCCGCCCCTACGAGGGCACCCAGACCGCCAAGCTGAAGGTGACCAAGG |
| | | GCGGCCCCCTGCCCTTCGCCTGGGACATCCTGTCCCCCCAGTTCATGTACGG |
| | | CTCCAAGGCGTACGTGAAGCACCCCGCCGACATCCCCGATTACAAGAAGCTG |
| | | TCCTTCCCCGAGGGCTTCAAGTGGGAGCGCGTGATGAACTTCGAGGACGGCG |
| | | GTCTGGTGACCGTGACCCAGGACTCCTCCCTGCAGGACGGCACGCTGATCTA |
| | | CAAGGTGAAGATGCGCGGCACCAACTTCCCCCCCGACGGCCCCGTAATGCAG |
| | | AAGAAGACCATGGGCTGGGAGGCCTCCACCGAGCGCCTGTACCCCCGCGAC |
| | | GGCGTGCTGAAGGGCGAGATCCACCAGGCCCTGAAGCTGAAGGACGGCGGC |
| | | CACTACCTGGTGGAGTTCAAGACCATCTACATGGCCAAGAAGCCCGTGCAACT |
| | | GCCCGGCTACTACTACGTGGACACCAAGCTGGACATCACCTCCCACAACGAG |
| | | GACTACACCATCGTGGAACAGTACGAGCGCTCCGAGGGCCGCCACCACCTGT |
| | | TCCTGGGGCATGGCACCGGCAGCACCGGCAGCGGCAGCTCCGGCACCGCCT |
| | | CCTCCGAGGACAACAACATGGCCGTCATCAAAGAGTTCATGCGCTTCAAGGTG |
| | | CGCATGGAGGGCTCCATGAACGGCCACGAGTTCGAGATCGAGGGCGAGGGC |
| | | GAGGGCCGCCCCTACGAGGGCACCCAGACCGCCAAGCTGAAGGTGACCAAG |
| | | GGCGGCCCCCTGCCCTTCGCCTGGGACATCCTGTCCCCCCAGTTCATGTACG |
| | | GCTCCAAGGCGTACGTGAAGCACCCCGCCGACATCCCCGATTACAAGAAGCT |
| | | GTCCTTCCCCGAGGGCTTCAAGTGGGAGCGCGTGATGAACTTCGAGGACGGC |
| | | GGTCTGGTGACCGTGACCCAGGACTCCTCCCTGCAGGACGGCACGCTGATCT |
| | | ACAAGGTGAAGATGCGCGGCACCAACTTCCCCCCCGACGGCCCCGTAATGCA |
| | | GAAGAAGACCATGGGCTGGGAGGCCTCCACCGAGCGCCTGTACCCCCGCGA |
| | | CGGCGTGCTGAAGGGCGAGATCCACCAGGCCCTGAAGCTGAAGGACGGCGG |
| | | CCGCTACCTGGTGGAGTTCAAGACCATCTACATGGCCAAGAAGCCCGTGCAA |
| | | CTGCCCGGCTACTACTACGTGGACACCAAGCTGGACATCACCTCCCACAACGA |
| | | GGACTACACCATCGTGGAACAGTACGAGCGCTCCGAGGGCCGCCACCACCTG |
| | | TTCCTGTACGGCATGGACGAGCTGTACAAGTAA |
| 60 | tdTomato | ATGTGGTGGCGCCTGTGGTGGCTGCTGCTGCTGCTGCTGCTGCTGTGGCCCA |
| | Secreted | TGGTGTGGGCCGTGAGCAAGGGCGAGGAGGTCATCAAAGAGTTCATGCGCTT |
| | | CAAGGTGCGCATGGAGGGCTCCATGAACGGCCACGAGTTCGAGATCGAGGG |
| | | CGAGGGCGAGGGCCGCCCCTACGAGGGCACCCAGACCGCCAAGCTGAAGGT |
| | | GACCAAGGGCGGCCCCCTGCCCTTCGCCTGGGACATCCTGTCCCCCCAGTTC |
| | | ATGTACGGCTCCAAGGCGTACGTGAAGCACCCCGCCGACATCCCCGATTACA |
| | | AGAAGCTGTCCTTCCCCGAGGGCTTCAAGTGGGAGCGCGTGATGAACTTCGA |
| | | GGACGGCGGTCTGGTGACCGTGACCCAGGACTCCTCCCTGCAGGACGGCAC |
| | | GCTGATCTACAAGGTGAAGATGCGCGGCACCAACTTCCCCCCCGACGGCCCC |
| | | GTAATGCAGAAGAAGACCATGGGCTGGGAGGCCTCCACCGAGCGCCTGTACC |
| | | CCCGCGACGGCGTGCTGAAGGGCGAGATCCACCAGGCCCTGAAGCTGAAGG |
| | | ACGGCGGCCACTACCTGGTGGAGTTCAAGACCATCTACATGGCCAAGAAGCC |
| | | CGTGCAACTGCCCGGCTACTACTACGTGGACACCAAGCTGGACATCACCTCC |
| | | CACAACGAGGACTACACCATCGTGGAACAGTACGAGCGCTCCGAGGGCCGCC |
| | | ACCACCTGTTCCTGGGGCATGGCACCGGCAGCACCGGCAGCGGCAGCTCCG |
| | | GCACCGCCTCCTCCGAGGACAACAACATGGCCGTCATCAAAGAGTTCATGCG |
| | | CTTCAAGGTGCGCATGGAGGGCTCCATGAACGGCCACGAGTTCGAGATCGAG |
| | | GGCGAGGGCGAGGGCCGCCCCTACGAGGGCACCCAGACCGCCAAGCTGAAG |
| | | GTGACCAAGGGCGGCCCCCTGCCCTTCGCCTGGGACATCCTGTCCCCCCAGT |
| | | TCATGTACGGCTCCAAGGCGTACGTGAAGCACCCCGCCGACATCCCCGATTA |
| | | CAAGAAGCTGTCCTTCCCCGAGGGCTTCAAGTGGGAGCGCGTGATGAACTTC |
| | | GAGGACGGCGGTCTGGTGACCGTGACCCAGGACTCCTCCCTGCAGGACGGC |
| | | ACGCTGATCTACAAGGTGAAGATGCGCGGCACCAACTTCCCCCCCGACGGCC |
| | | CCGTAATGCAGAAGAAGACCATGGGCTGGGAGGCCTCCACCGAGCGCCTGTA |
| | | CCCCCGCGACGGCGTGCTGAAGGGCGAGATCCACCAGGCCCTGAAGCTGAA |
| | | GGACGGCGGCCGCTACCTGGTGGAGTTCAAGACCATCTACATGGCCAAGAAG |
| | | CCCGTGCAACTGCCCGGCTACTACTACGTGGACACCAAGCTGGACATCACCT |
| | | CCCACAACGAGGACTACACCATCGTGGAACAGTACGAGCGCTCCGAGGGCCG |
| | | CCACCACCTGTTCCTGTACGGCATGGACGAGCTGTACAAGTAA |
| 61 | tdTomato | ATGGGCTGCGTGTGCAGCAGCAACCCCGAGAACAACAACAACATGGTGAGCA |
| | Membrane | AGGGCGAGGAGGTCATCAAAGAGTTCATGCGCTTCAAGGTGCGCATGGAGGG |
| | | CTCCATGAACGGCCACGAGTTCGAGATCGAGGGCGAGGGCGAGGGCCGCCC |
| | | CTACGAGGGCACCCAGACCGCCAAGCTGAAGGTGACCAAGGGCGGCCCCCT |
| | | GCCCTTCGCCTGGGACATCCTGTCCCCCCAGTTCATGTACGGCTCCAAGGCG |
| | | TACGTGAAGCACCCCGCCGACATCCCCGATTACAAGAAGCTGTCCTTCCCCGA |
| | | GGGCTTCAAGTGGGAGCGCGTGATGAACTTCGAGGACGGCGGTCTGGTGACC |
| | | GTGACCCAGGACTCCTCCCTGCAGGACGGCACGCTGATCTACAAGGTGAAGA |
| | | TGCGCGGCACCAACTTCCCCCCCGACGGCCCCGTAATGCAGAAGAAGACCAT |
| | | GGGCTGGGAGGCCTCCACCGAGCGCCTGTACCCCCGCGACGGCGTGCTGAA |
| | | GGGCGAGATCCACCAGGCCCTGAAGCTGAAGGACGGCGGCCACTACCTGGT |
| | | GGAGTTCAAGACCATCTACATGGCCAAGAAGCCCGTGCAACTGCCCGGCTAC |
| | | TACTACGTGGACACCAAGCTGGACATCACCTCCCACAACGAGGACTACACCAT |
| | | CGTGGAACAGTACGAGCGCTCCGAGGGCCGCCACCACCTGTTCCTGGGGCAT |
| | | GGCACCGGCAGCACCGGCAGCGGCAGCTCCGGCACCGCCTCCTCCGAGGAC |
| | | AACAACATGGCCGTCATCAAAGAGTTCATGCGCTTCAAGGTGCGCATGGAGG |
| | | GCTCCATGAACGGCCACGAGTTCGAGATCGAGGGCGAGGGCGAGGGCCGCC |
| | | CCTACGAGGGCACCCAGACCGCCAAGCTGAAGGTGACCAAGGGCGGCCCCC |
| | | TGCCCTTCGCCTGGGACATCCTGTCCCCCCAGTTCATGTACGGCTCCAAGGC |
| | | GTACGTGAAGCACCCCGCCGACATCCCCGATTACAAGAAGCTGTCCTTCCCC |
| | | GAGGGCTTCAAGTGGGAGCGCGTGATGAACTTCGAGGACGGCGGTCTGGTGA |
| | | CCGTGACCCAGGACTCCTCCCTGCAGGACGGCACGCTGATCTACAAGGTGAA |
| | | GATGCGCGGCACCAACTTCCCCCCCGACGGCCCCGTAATGCAGAAGAAGACC |
| | | ATGGGCTGGGAGGCCTCCACCGAGCGCCTGTACCCCCGCGACGGCGTGCTG |
| | | AAGGGCGAGATCCACCAGGCCCTGAAGCTGAAGGACGGCGGCCGCTACCTG |
| | | GTGGAGTTCAAGACCATCTACATGGCCAAGAAGCCCGTGCAACTGCCCGGCT |
| | | ACTACTACGTGGACACCAAGCTGGACATCACCTCCCACAACGAGGACTACACC |
| | | ATCGTGGAACAGTACGAGCGCTCCGAGGGCCGCCACCACCTGTTCCTGTACG |
| | | GCATGGACGAGCTGTACAAGTAA |
| 62 | eGFP | ATGGTGAGCAAGGGCGAGGAGCTGTTCACCGGGGTGGTGCCCATCCTGGTC |
| | | GAGCTGGACGGCGACGTAAACGGCCACAAGTTCAGCGTGTCCGGCGAGGGC |
| | | GAGGGCGATGCCACCTACGGCAAGCTGACCCTGAAGTTCATCTGCACCACCG |
| | | GCAAGCTGCCCGTGCCCTGGCCCACCCTCGTGACCACCCTGACCTACGGCGT |
| | | GCAGTGCTTCAGCCGCTACCCCGACCACATGAAGCAGCACGACTTCTTCAAGT |
| | | CCGCCATGCCCGAAGGCTACGTCCAGGAGCGCACCATCTTCTTCAAGGACGA |
| | | CGGCAACTACAAGACCCGCGCCGAGGTGAAGTTCGAGGGCGACACCCTGGT |
| | | GAACCGCATCGAGCTGAAGGGCATCGACTTCAAGGAGGACGGCAACATCCTG |
| | | GGGCACAAGCTGGAGTACAACTACAACAGCCACAACGTCTATATCATGGCCGA |
| | | CAAGCAGAAGAACGGCATCAAGGTGAACTTCAAGATCCGCCACAACATCGAG |
| | | GACGGCAGCGTGCAGCTCGCCGACCACTACCAGCAGAACACCCCCATCGGC |
| | | GACGGCCCCGTGCTGCTGCCCGACAACCACTACCTGAGCACCCAGTCCGCCC |
| | | TGAGCAAAGACCCCAACGAGAAGCGCGATCACATGGTCCTGCTGGAGTTCGT |
| | | GACCGCCGCCGGGATCACTCTCGGCATGGACGAGCTGTACAAGTAA |
| 63 | IL-2 | ATGTACAGGATGCAACTCCTGTCTTGCATTGCACTAAGTCTTGCACTTGTCACA |
| | | AACAGTGCACCCACCAGCAGCAGCACCAAGAAGACCCAGCTGCAGCTGGAGC |
| | | ACCTGCTGCTGGACCTGCAGATGATCCTGAACGGCATCAACAACTACAAGAAC |
| | | CCCAAGCTGACCAGAATGCTGACCTTCAAGTTCTACATGCCCAAGAAGGCCAC |
| | | CGAGCTGAAGCACCTGCAGTGCCTGGAGGAGGAGCTGAAGCCCCTGGAGGA |
| | | GGTGCTGAACCTGGCCCAGAGCAAGAACTTCCACCTGAGACCCAGAGACCTG |
| | | ATCAGCAACATCAACGTGATCGTGCTGGAGCTGAAGGGCAGCGAGACCACCT |
| | | TCATGTGCGAGTACGCCGACGAGACCGCCACCATCGTGGAGTTCCTGAACAG |
| | | ATGGATCACCTTCAGCCAGAGCATCATCAGCACCCTGACCTGA |
| 64 | sumIL-2 | ATGTACAGGATGCAACTCCTGTCTTGCATTGCACTAAGTCTTGCACTTGTCACA |
| | | AACAGTGCACCCACCAGCAGCAGCACCAAGAAGACCCAGCTGCAGCTGGAGC |
| | | ACCTGCTGCTGGACCTGCAGATGATCCTGAACGGCATCAACAACTACAAGAAC |
| | | CCCAAGCTGACCAGAATGCTGACCGCCAAGTTCTACATGCCCAAGAAGGCCA |
| | | CCGAGCTGAAGCACCTGCAGTGCCTGGAGGAGGAGCTGAAGCCCCTGGAGG |
| | | AGGTGCTGAACCTGGCCCAGAGCAAGAACTTCCACTTCGACCCCAGAGACGT |
| | | GGTGAGCAACATCAACGTGTTCGTGCTGGAGCTGAAGGGCAGCGAGACCACC |
| | | TTCATGTGCGAGTACGCCGACGAGACCGCCACCATCGTGGAGTTCCTGAACA |
| | | GATGGATCACCTTCAGCCAGAGCATCATCAGCACCCTGACCTGA |
| 65 | IL-2 + | ATGTACAGGATGCAACTCCTGTCTTGCATTGCACTAAGTCTTGCACTTGTCACA |
| | IL-2-Ra | AACAGTAGCAAGAACTTCCACCTGAGACCCAGAGACCTGATCAGCAACATCAA |
| | | CGTGATCGTGCTGGAGCTGAAGGGCAGCGAGACCACCTTCATGTGCGAGTAC |
| | | GCCGACGAGACCGCCACCATCGTGGAGTTCCTGAACAGATGGATCACCTTCA |
| | | GCCAGAGCATCATCAGCACCCTGACCGGCGGCAGCAGCAGCACCAAGAAGAC |
| | | CCAGCTGCAGCTGGAGCACCTGCTGCTGGACCTGCAGATGATCCTGAACGGC |
| | | ATCAACAACTACAAGAACCCCAAGCTGACCAGAATGCTGACCTTCAAGTTCTAC |
| | | ATGCCCAAGAAGGCCACCGAGCTGAAGCACCTGCAGTGCCTGGAGGAGGAG |
| | | CTGAAGCCCCTGGAGGAGGTGCTGAACCTGGCCCAGGGCAGCGGCGGCGGC |
| | | AGCGAGCTGTGCGACGACGACCCCCCCGAGATCCCCCACGCCACCTTCAAGG |
| | | CCATGGCCTACAAGGAGGGCACCATGCTGAACTGCGAGTGCAAGAGAGGCTT |
| | | CAGAAGAATCAAGAGCGGCAGCCTGTACATGCTGTGCACCGGCAACAGCAGC |
| | | CACAGCAGCTGGGACAACCAGTGCCAGTGCACCAGCAGCGCCACCAGAAACA |
| | | CCACCAAGCAGGTGACCCCCCAGCCCGAGGAGCAGAAGGAGAGAAAGACCA |
| | | CCGAGATGCAGAGCCCCATGCAGCCCGTGGACCAGGCCAGCCTGCCCGGCC |
| | | ACTGCAGAGAGCCCCCCCCCTGGGAGAACGAGGCCACCGAGAGAATCTACCA |
| | | CTTCGTGGTGGGCCAGATGGTGTACTACCAGTGCGTGCAGGGCTACAGAGCC |
| | | CTGCACAGAGGCCCCGCCGAGAGCGTGTGCAAGATGACCCACGGCAAGACC |
| | | AGATGGACCCAGCCCCAGCTGATCTGCACCGGCTGA |
| 66 | Anti-CD19 & | ATGCTGCTGCTGGTGACCAGCCTGCTGCTGTGCGAGCTGCCCCACCCCGCCT |
| | CD20 CAR-T | TCCTTTTGATTCCAGAGGTGCAATTACAGCAAAGCGGTGCAGAACTCGTGAAG |
| | | CCAGGCGCTTCAGTAAAAATGTCCTGCAAAGCTTCCGGCTACACATTCACTAG |
| | | CTACAACATGCATTGGGTTAAACAAACACCTGGCCAGGGATTGGAGTGGATCG |
| | | GGGCTATATATCCGGGTAACGGTGACACTAGTTACAATCAGAAATTCAAGGGA |
| | | AAGGCCACGCTTACAGCAGACAAGAGTAGCAGTACCGCCTATATGCAACTCAG |
| | | CAGCTTGACCAGCGAAGACAGCGCAGATTATTACTGTGCCAGATCCAACTATT |
| | | ACGGCTCAAGCTACTGGTTCTTTGACGTGTGGGGCGCCGGAACCACTGTTACT |
| | | GTCAGCAGCGGAGGCGGTGGGAGTGGCGGCGGTGGTTCCGGCGGGGGGCGG |
| | | GTCAGATATTGTGTTGACGCAATCCCCCGCAATCCTGTCCGCGTCCCCAGGCG |
| | | AAAAAGTGACAATGACATGCCGGGCCAGCTCAAGCGTGAATTACATGGATTGG |
| | | TATCAGAAGAAACCCGGGAGCAGCCCCAAGCCGTGGATCTATGCGACGAGTA |
| | | ACCTGGCTTCCGGCGTCCCCGCCCGATTCAGCGGGTCCGGCAGCGGCACTA |
| | | GTTACAGTTTAACCATCAGCAGGGTGGAGGCTGAAGACGCGGCAACCTATTAT |
| | | TGCCAGCAGTGGTCATTTAATCCTCCCACGTTTGGCGGCGGCACCAAGCTGG |
| | | AGATAAAGGGCGGGGGGGGGCAGTGGAGGCGGAGGTAGCGGCGGAGGAGGA |
| | | TCAGGAGGGGGGGGTAGCGGAGGAGGGGGTTCAGACATACAGATGACACAG |
| | | ACAACCTCGAGCCTGAGCGCATCCCTGGGGGACCGAGTCACCATCTCATGTA |
| | | GAGCATCGCAGGACATCAGCAAATATCTCAACTGGTACCAGCAAAAACCCGAT |
| | | GGTACAGTGAAACTCTTAATTTACCACACATCCCGGTTACATAGCGGAGTCCC |
| | | AAGCCGCTTTAGCGGATCCGGAAGCGGGACCGACTACTCCCTCACGATCAGC |
| | | AACCTCGAACAGGAGGATATAGCCACTTATTTCTGCCAGCAGGGTAACACCCT |
| | | GCCGTATACATTTGGCGGGGGCACAAAACTAGAAATCACAGGCAGTACAAGTG |
| | | GTAGTGGGAAGCCCGGTAGCGGAGAAGGTAGCACGAAAGGCGAGGTTAAACT |
| | | TCAGGAATCAGGCCCAGGCCTAGTTGCCCCTTCACAAAGTCTAAGCGTCACGT |
| | | GTACCGTGTCCGGCGTAAGCCTGCCAGATTATGGAGTCAGCTGGATTAGGCA |
| | | GCCTCCACGGAAGGGACTGGAGTGGCTGGGCGTGATTTGGGGGTCGGAGAC |
| | | CACCTATTATAACAGCGCGCTGAAATCGCGGCTGACTATTATTAAGGATAATAG |
| | | CAAGTCACAGGTGTTTCTTAAGATGAATAGCCTGCAGACTGATGACACTGCGA |
| | | TATACTACTGTGCCAAGCACTACTACTACGGAGGGTCATATGCTATGGATTACT |
| | | GGGGGCAGGGAACCTCCGTGACTGTAAGTTCCGCCGCCGCTACTACAACCCC |
| | | TGCCCCACGTCCCCCGACCCCAGCACCTACTATCGCATCCCAGCCCCTTTCCC |
| | | TTCGCCCTGAGGCCTGCAGGCCTGCAGCTGGAGGAGCCGTTCACACCAGGG |
| | | GCCTCGACTTTGCATGCGATATTTACATCTGGGCCCCACTGGCTGGCACCTGC |
| | | GGCGTCTTGCTGCTGAGCCTCGTGATCACCCTCTATTGTAAGAGGGGGCGAA |
| | | AAAAGCTCCTGTACATTTTCAAACAGCCTTTCATGAGACCTGTGCAGACAACAC |
| | | AGGAAGAGGACGGTTGCTCCTGTAGGTTTCCGGAGGAAGAAGAGGGAGGGTG |
| | | TGAATTGCGTGTAAAGTTCTCCAGATCAGCTGATGCCCCAGCCTATCAACAAG |
| | | GACAGAATCAGCTGTACAACGAGCTTAATTTGGGGCGGAGAGAGGAATACGAT |
| | | GTCTTGGACAAGAGACGGGGGCGCGACCCCGAGATGGGCGGGAAGCCTAGA |
| | | AGGAAGAACCCTCAGGAGGGGCTGTATAATGAGCTCCAAAAGGATAAGATGG |
| | | CCGAAGCTTACAGCGAGATCGGGATGAAAGGAGAGCGCCGCCGCGGAAAAG |
| | | GCCATGATGGGTTATACCAGGGCCTTAGCACCGCCACTAAGGACACTTACGAC |
| | | GCTCTACACATGCAGGCTCTCCCACCCCGATGA |
| 67 | Anti-HER2 | ATGAGGGCTTGGATCTTCTTTCTGCTCTGCCTGGCCGGGCGCGCCTTGGCCG |
| | Heavy Chain | AAGTTCAACTGGTAGAGAGTGGAGGTGGGCTTGTGCAACCAGGCGGATCCTT |
| | | GCGACTGTCCTGCGCCGCTTCAGGCTTCAACATCAAGGACACCTACATCCATT |
| | | GGGTCCGCCAGGCACCAGGAAAAGGTCTTGAATGGGTGGCCAGAATCTACCC |
| | | TACTAACGGTTACACCAGATATGCAGACTCCGTTAAGGGGCGATTTACCATTTC |
| | | AGCAGACACCTCTAAGAACACCGCTTACCTGCAGATGAACTCACTTCGAGCTG |
| | | AGGACACCGCCGTTTACTATTGCAGCAGATGGGGCGGTGACGGCTTCTACGC |
| | | TATGGATTACTGGGGACAGGGGACTTTGGTAACTGTGAGTAGTGCATCTACAA |
| | | AGGGGCCAAGCGTGTTCCCACTTGCCCCATCTTCTAAAAGCACCTCAGGAGG |
| | | GACTGCAGCCTTGGGTTGCTTGGTTAAAGATTATTTTCCAGAGCCTGTAACTGT |
| | | ATCCTGGAATAGTGGGGCCCTCACAAGCGGAGTACATACTTTCCCTGCAGTAT |
| | | TGCAGTCTAGTGGACTCTACTCTCTCAGCAGTGTAGTGACCGTACCTTCCAGT |
| | | TCACTTGGAACACAGACCTATATTTGCAATGTGAATCATAAGCCATCTAATACT |
| | | AAAGTGGATAAGAAAGTGGAGCCTAAATCTTGTGACAAGACTCATACATGCCC |
| | | TCCCTGCCCTGCCCCTGAACTGTTGGGAGGGCCCTCTGTATTTCTCTTCCCCC |
| | | CTAAACCAAAGGACACCCTGATGATCAGTCGAACTCCTGAGGTGACTTGTGTG |
| | | GTTGTTGACGTGTCACATGAGGATCCCGAAGTGAAATTCAACTGGTACGTCGA |
| | | TGGAGTAGAGGTACACAATGCAAAGACAAAACCTAGGGAGGAACAGTATAATT |
| | | CTACCTATAGAGTGGTGTCTGTTCTCACAGTTCTCCATCAAGACTGGTTGAACG |
| | | GTAAAGAATATAAATGCAAAGTCTCCAATAAGGCTTTGCCCGCTCCCATTGAAA |
| | | AAACAATCAGTAAAGCCAAAGGCCAGCCACGCGAACCACAGGTCTACACCCTT |
| | | CCACCATCTCGCGAAGAAATGACTAAGAATCAAGTGTCACTCACATGCCTCGT |
| | | CAAGGGCTTTTACCCTTCTGATATTGCAGTTGAATGGGAGTCTAACGGGCAGC |
| | | CCGAGAATAATTACAAGACTACCCCCCCCGTCCTTGACTCTGATGGCAGCTTT |
| | | TTCCTGTACTCAAAATTGACTGTGGACAAGTCTCGATGGCAACAGGGTAATGTT |
| | | TTCTCCTGTAGCGTAATGCACGAGGCCCTTCATAACCATTATACCCAAAAATCT |
| | | CTTTCATTGTCCCCTGGAAAATGA |
| 68 | Anti-HER2 | ATGAGGGCTTGGATCTTCTTTCTGCTCTGCCTGGCCGGGCGCGCCTTGGCCG |
| | Light | ATATTCAGATGACTCAGAGCCCCAGCAGCCTGTCTGCTAGCGTTGGGGATAGA |
| | Chain | GTCACTATAACATGTCGGGCTTCCCAGGATGTGAATACTGCTGTCGCTTGGTA |
| | | TCAACAGAAGCCCGGCAAGGCACCAAAACTGCTGATATATAGTGCCTCTTTCC |
| | | TTTACTCCGGGGTTCCCAGTCGATTCTCTGGAAGCCGCAGCGGCACTGATTTC |
| | | ACACTTACTATAAGTAGTCTGCAACCTGAGGACTTTGCTACATACTACTGCCAG |
| | | CAGCACTATACAACACCCCCAACTTTCGGGCAAGGCACTAAGGTAGAAATTAA |
| | | AAGGACCGTTGCTGCTCCATCCGTCTTTATTTTTCCACCATCTGATGAACAGTT |
| | | GAAGAGTGGAACAGCAAGCGTCGTTTGTCTCCTGAACAATTTTTACCCACGAG |
| | | AGGCAAAAGTTCAATGGAAGGTAGACAATGCCCTTCAGAGCGGCAATTCCCAG |
| | | GAGAGCGTAACCGAGCAGGATAGCAAAGACTCTACATACTCTTTGAGTTCAAC |
| | | CCTTACCCTGAGCAAGGCTGATTACGAAAAACACAAGGTGTACGCTTGCGAGG |
| | | TAACCCATCAGGGATTGTCATCCCCAGTCACAAAATCATTCAATAGGGGCGAG |
| | | TGCTGA |
| 69 | Keytruda | AUGGAAUUUGGACUGUCUUGGGUUUUUCUGGUAGCUCUGUUUCGUGGAGU |
| | | GCAAUGCCAGGUUCAGUUGGUCCAGUCGGGUGUGGAGGUUAAAAAGCCCG |
| | | GCGCCUCUGUGAAGGUUAGCUGCAAGGCUAGCGGAUAUACUUUCACCAAUU |
| | | ACUACAUGUAUUGGGUGAGGCAGGCUCCAGGGCAGGGACUCGAGUGGAUG |
| | | GGAGGCAUCAACCCGUCCAACGGCGGCACCAACUUCAAUGAGAAGUUCAAA |
| | | AAUAGAGUGACUCUCACCACUGAUAGUUCCACGACAACCGCAUACAUGGAAC |
| | | UGAAAAGUCUGCAAUUCGAUGACACCGCCGUGUAUUACUGCGCCCGCAGAG |
| | | ACUACCGGUUUGACAUGGGAUUUGAUUACUGGGGUCAGGGCACCACAGUGA |
| | | CAGUCUCAUCCGCCUCUACCAAAGGACCCUCGGUUUUUCCACUCGCACCUU |
| | | GCUCCCGAAGCACCUCAGAGUCCACCGCGGCCCUUGGGUGUCUGGUGAAA |
| | | GAUUAUUUCCCCGAACCAGUGACCGUGUCAUGGAAUUCAGGGGCACUGACA |
| | | AGUGGGGUCCACACGUUCCCAGCGGUGCUGCAGUCCUCCGGGCUGUAUAG |
| | | CCUUAGCUCUGUAGUCACUGUCCCGUCCAGUUCCCUGGGGACUAAGACUUA |
| | | CACAUGUAACGUAGAUCACAAACCUAGUAACACCAAGGUGGACAAGCGCGU |
| | | GGAGUCAAAGUACGGACCUCCAUGUCCCCCAUGUCCCGCCCCCGAAUUUUU |
| | | GGGAGGACCUUCAGUGUUCCUGUUCCCUCCAAAACCUAAAGACACACUGAU |
| | | GAUAAGCAGGACACCCGAGGUCACCUGCGUCGUGGUCGACGUAAGCCAGGA |
| | | AGAUCCAGAGGUACAGUUCAACUGGUACGUCGACGGUGUAGAGGUCCAUAA |
| | | UGCCAAGACAAAGCCCAGGGAGGAGCAGUUUAACUCAACAUAUCGUGUUGU |
| | | GUCUGUGUUGACGGUUCUACAUCAAGACUGGCUAAACGGCAAGGAGUAUAA |
| | | AUGCAAGGUUAGUAAUAAGGGCCUCCCCAGCUCUAUUGAGAAAACUAUCAG |
| | | CAAAGCUAAGGGCCAGCCUCGGGAACCACAAGUUUACACUUUACCGCCCAG |
| | | CCAGGAGGAAAUGACGAAGAAUCAGGUGAGCCUGACAUGUCUUGUCAAGGG |
| | | GUUUUACCCCUCUGAUAUCGCUGUAGAAUGGGAAUCCAAUGGUCAACCUGA |
| | | AAACAACUACAAGACCACCCCGCCUGUGCUCGACUCUGACGGCAGCUUUUU |
| | | CCUGUAUUCGAGACUUACAGUGGAUAAAAGCCGGUGGCAGGAGGGCAAUGU |
| | | GUUUUCCUGCAGUGUGAUGCACGAAGCAUUACACAACCAUUAUACUCAAAAA |
| | | UCACUCAGUUUGUCUCUGGGGAAG |
| 70 | Epogen | AUGGGGGUCCACGAAUGCCCGGCAUGGCUGUGGUUACUGCUAUCGCUGCU |
| | | CAGCCUGCCACUGGGCCUGCCCGUACUUGGCGCCCCUCCACGCCUCAUCU |
| | | GUGACUCCCGGGUCCUGGAGCGGUACCUUUUGGAGGCCAAAGAAGCAGAG |
| | | AAUAUCACUACAGGCUGCGCUGAGCACUGUAGUUUGAACGAAAAUAUAACAG |
| | | UACCUGACACAAAGGUGAAUUUUUACGCCUGGAAAAGAAUGGAGGUGGGCC |
| | | AACAGGCAGUGGAAGUGUGGCAGGGUCUCGCUUUACUUAGCGAGGCCGUG |
| | | CUGCGAGGUCAGGCACUCCUAGUUAACUCCAGCCAGCCCUGGGAGCCACUC |
| | | CAGUUGCAUGUGGAUAAGGCCGUCUCUGGACUAAGAUCCCUCACCACCCUG |
| | | CUGAGGGCAUUGGGAGCGCAAAAGGAGGCUAUUUCACCUCCCGAUGCCGCU |
| | | AGUGCUGCGCCCCUUCGCACUAUCACCGCCGACACGUUCCGUAAGCUGUUC |
| | | AGGGUUUAUUCAAACUUUCUCCGGGGGAAGCUGAAACUGUAUACUGGGGAA |
| | | GCCUGCAGAACCGGAGAUAGG |
| 71 | Enbrel | AUGAAAUGGGUUACGUUCAUCUCCUUGCUGUUCCUGUUCUCUUCCGCUUAC |
| | | UCCCUGCCAGCCCAGGUCGCUUUCACGCCAUACGCUCCUGAACCAGGUAGC |
| | | ACUUGUCGUCUACGGGAAUACUAUGACCAGACUGCACAGAUGUGCUGUUCC |
| | | AAGUGCUCUCCUGGCCAACAUGCCAAAGUGUUUUGCACUAAGACUAGCGAU |
| | | ACAGUGUGCGAUAGCUGCGAGGAUUCUACAUAUACCCAGCUCUGGAACUGG |
| | | GUUCCCGAGUGUCUCAGCUGUGGCAGCCGGUGUAGUAGCGACCAGGUGGA |
| | | GACCCAGGCCUGCACACGAGAGCAGAACCGGAUCUGCACAUGUAGACCCGG |
| | | GUGGUACUGUGCGUUGAGUAAACAGGAGGGAUGUAGACUCUGCGCCCCUC |
| | | UUAGGAAGUGCCGCCCAGGGUUUGGAGUCGCACGCCCAGGCACAGAAACCA |
| | | GUGACGUCGUCUGCAAGCCGUGUGCCCCGGGCACAUUCUCCAACACUACCU |
| | | CAAGUACAGAUAUUUGUAGACCUCAUCAGAUCUGCAAUGUCGUGGCAAUUC |
| | | CCGGUAACGCCUCGAUGGAUGCCGUGUGCACCUCUACGUCGCCCACUAGGU |
| | | CCAUGGCCCCAGGAGCAGUACACUUACCUCAGCCUGUUAGCACACGCUCCC |
| | | AGCACACGCAACCCACCCCUGAGCCUUCCACUGCACCGUCCACCAGCUUCC |
| | | UGCUGCCCAUGGGCCCAAGUCCUCCAGCGGAGGGCAGCACAGGGGACGAG |
| | | CCAAAGUCCUGCGACAAAACCCAUACUUGUCCCCCUUGCCCCGCACCCGAA |
| | | CUCCUUGGGGGACCAUCAGUAUUCUUGUUUCCCCCCAAGCCAAAGGACACA |
| | | UUAAUGAUUUCUAGGACUCCGGAGGUGACUUGCGUCGUUGUUGACGUGAG |
| | | CCACGAAGACCCAGAGGUGAAAUUCAACUGGUAUGUGGAUGGAGUCGAAGU |
| | | UCACAAUGCGAAAACCAAACCUCGAGAGGAACAGUACAACUCAACCUAUAGG |
| | | GUAGUGAGUGUUCUGACCGUGCUGCACCAGGACUGGCUCAAUGGGAAGGA |
| | | AUACAAGUGUAAGGUAUCCAAUAAAGCCCUGCCUGCCCCUAUCGAAAAGACA |
| | | AUAAGCAAGGCUAAGGGGCAACCUCGCGAACCCCAGGUGUACACGCUGCCA |
| | | CCAAGCAGAGAGGAGAUGACAAAAAACCAAGUGUCACUGACCUGCCUGGUG |
| | | AAAGGCUUUUACCCGAGCGAUAUCGCUGUGGAGUGGGAGUCAAACGGACAA |
| | | CCCGAGAAUAACUAUAAAACCACGCCCCCCGUGCUGGAUUCAGACGGUUCU |
| | | UUCUUCCUUUAUUCCAAGUUGACCGUGGACAAGUCUCGGUGGCAGCAGGGC |
| | | AAUGUCUUUUCCUGUAGUGUCAUGCACGAAGCUCUACACAAUCAUUACACCC |
| | | AAAAAAGCCUGUCACUCUCUCCCGGUAAA |
| 72 | Filgrastim | AUGGCAGGCCCAGCUACGCAGAGCCCAAUGAAACUGAUGGCACUGCAGCUC |
| | | CUGUUAUGGCACAGCGCCCUGUGGACCGUCCAGGAAGCAACACCCCUCGGG |
| | | CCUGCGUCCAGUCUCCCCCAGAGCUUUCUACUUAAAUGUCUAGAGCAGGUG |
| | | CGGAAGAUCCAAGGAGACGGGGCCGCACUGCAGGAGAAGCUCUGCGCUACA |
| | | UACAAGCUUUGUCACCCAGAGGAACUGGUGCUGUUAGGCCAUUCACUGGGA |
| | | AUCCCUUGGGCCCCUUUGUCUAGUUGCCCGUCCCAGGCUUUACAGUUGGC |
| | | CGGCUGCCUGAGCCAGCUGCACUCCGGACUAUUUCUGUAUCAGGGACUUUU |
| | | GCAGGCCCUGGAGGGUAUUUCCCCUGAGCUCGGGCCGACUCUGGAUACAC |
| | | UGCAGCUUGAUGUCGCCGACUUUGCUACUACCAUAUGGCAACAAAUGGAGG |
| | | AACUCGGUAUGGCCCCAGCGCUGCAACCCACCCAGGGGGCCAUGCCCGCUU |
| | | UCGCGUCUGCCUUCCAGCGCAGAGCAGGCGGCGUGUUGGUGGCCUCACAU |
| | | CUCCAAUCGUUCCUGGAAGUUUCUUACCGAGUACUCAGGCACUUGGCACAG |
| | | CCC |
| 73 | Humira | AUGGAAUUUGGACUGUCUUGGGUUUUUCUGGUAGCUCUGUUUCGUGGAGU |
| | | GCAAUGCGAGGUUCAGCUUGUCGAGAGCGGCGGCGGUCUCGUGCAACCCG |
| | | GACGGAGUCUGAGACUCAGCUGUGCUGCUUCAGGAUUUACCUUCGACGAUU |
| | | AUGCGAUGCAUUGGGUGCGGCAGGCUCCUGGCAAAGGAUUAGAAUGGGUG |
| | | AGCGCAAUUACAUGGAACUCUGGCCACAUCGACUACGCCGAUUCAGUGGAG |
| | | GGCAGGUUCACCAUUAGCAGAGAUAAUGCUAAGAAUAGCUUAUACUUGCAG |
| | | AUGAACAGCCUGCGCGCCGAGGAUACUGCGGUGUACUAUUGCGCCAAGGUC |
| | | AGUUAUCUGUCCACCGCCAGCUCCCUAGAUUAUUGGGGGCAGGGAACCUUG |
| | | GUGACAGUCAGUUCCGCAAGCACGAAAGGGCCCAGCGUUUUCCCUCUCGCA |
| | | CCCUCUUCAAAAUCCACUUCAGGGGGGACAGCUGCACUGGGAUGUCUUGUC |
| | | AAGGAUUACUUCCCCGAACCUGUGACCGUCUCAUGGAAUUCCGGUGCCUUG |
| | | ACCUCAGGGGUGCAUACCUUCCCCGCAGUCCUUCAGAGUUCUGGACUCUAC |
| | | UCCCUGAGUUCUGUGGUAACAGUCCCUUCUAGCUCCCUCGGGACACAGACC |
| | | UACAUUUGCAAUGUGAACCAUAAACCGAGUAACACUAAAGUGGACAAAAAAG |
| | | UAGAACCCAAAAGCUGCGAUAAGACCCACACGUGCCCCCCGUGUCCGGCCC |
| | | CCGAGCUUCUGGGCGGCCCUUCGGUGUUCCUGUUUCCACCUAAGCCCAAAG |
| | | ACACACUGAUGAUCUCUCGCACUCCUGAAGUAACAUGCGUGGUGGUCGACG |
| | | UGAGCCACGAAGAUCCAGAAGUUAAGUUCAAUUGGUAUGUUGACGGGGUGG |
| | | AGGUGCACAACGCCAAGACUAAGCCAAGGGAGGAGCAAUACAAUUCCACAUA |
| | | CCGAGUUGUCAGCGUACUUACGGUUCUGCACCAAGACUGGCUAAAUGGUAA |
| | | AGAGUACAAGUGUAAGGUGUCUAACAAAGCCCUGCCAGCGCCGAUAGAAAA |
| | | GACUAUCUCAAAAGCAAAGGGCCAGCCCAGAGAACCUCAGGUGUACACCUU |
| | | GCCACCAUCAAGGGACGAGCUCACCAAGAAUCAGGUUAGUCUCACCUGUCU |
| | | GGUGAAGGGGUUCUAUCCAUCCGACAUCGCCGUGGAGUGGGAGAGUAACG |
| | | GCCAGCCCGAAAACAAUUACAAGACGACACCACCAGUCUUGGAUAGCGACG |
| | | GUUCUUUCUUUCUAUAUUCGAAGCUCACUGUCGACAAGUCGCGGUGGCAGC |
| | | AGGGCAACGUAUUUAGCUGCUCCGUCAUGCAUGAGGCCCUGCACAACCACU |
| | | AUACUCAAAAAUCCCUGUCCUUAUCUCCUGGCAAG |
| 74 | Rituximab | AUGAAGUAUCUACUGCCUACCGCUGCAGCUGGGCUGUUGCUUCUGGCUGC |
| | | UCAACCAGCCAUGGCUCAGAUCGUGCUCUCCCAGUCCCCCGCGAUUUUGAG |
| | | CGCAUCCCCAGGCGAGAAGGUAACAAUGACGUGCCGCGCGAGCAGUUCAGU |
| | | GUCUUACAUCCACUGGUUCCAGCAAAAGCCAGGCUCUAGCCCCAAGCCCUG |
| | | GAUUUACGCAACCAGCAAUCUUGCCAGUGGCGUCCCGGUGAGGUUCUCGG |
| | | GGUCUGGAAGCGGUACAUCUUAUAGUCUCACCAUAAGCAGGGUAGAGGCCG |
| | | AGGACGCCGCUACUUACUAUUGCCAGCAGUGGACCUCUAACCCACCCACCU |
| | | UCGGCGGUGGAACCAAGCUGGAAAUCAAACGAACAGUCGCCGCCCCGUCCG |
| | | UGUUUAUCUUCCCUCCUUCUGACGAGCAGCUGAAAUCCGGGACCGCAUCCG |
| | | UCGUUUGUCUACUGAACAACUUUUACCCCCGGGAAGCAAAAGUCCAGUGGA |
| | | AGGUUGAUAACGCCCUCCAAAGUGGAAAUAGCCAGGAAUCAGUGACUGAGC |
| | | AAGAUUCCAAAGAUAGUACUUACAGCUUAUCAAGCACGCUCACUCUGUCCAA |
| | | AGCCGACUAUGAAAAACACAAGGUGUACGCCUGUGAGGUGACACAUCAGGG |
| | | GCUGUCAUCACCUGUUACAAAGUCGUUUAAUAGAGGCGAAUGC |
| 75 | Romiplostim | AUGAAAUGGGUUACGUUCAUCUCCUUGCUGUUCCUGUUCUCUUCCGCUUAC |
| | | UCCGAUAAAACACAUACCUGUCCCCCAUGUCCCGCCCCCGAGCUCCUCGGC |
| | | GGUCCCAGUGUGUUUCUGUUUCCACCAAAACCCAAGGACACUCUCAUGAUA |
| | | AGCCGCACGCCCGAGGUUACAUGCGUCGUCGUCGACGUCUCCCACGAGGAU |
| | | CCGGAGGUGAAAUUUAACUGGUAUGUAGAUGGCGUGGAAGUGCACAACGCA |
| | | AAAACCAAGCCCCGUGAAGAACAAUACAACUCCACAUACAGGGUCGUGAGCG |
| | | UGCUGACUGUACUCCAUCAGGAUUGGUUAAAUGGCAAGGAGUAUAAGUGUA |
| | | AGGUGAGUAAUAAGGCCCUACCUGCACCUAUUGAAAAGACAAUUAGCAAAGC |
| | | CAAGGGCCAGCCGAGGGAGCCACAGGUGUAUACACUGCCACCCAGCCGGGA |
| | | UGAGUUAACAAAGAAUCAGGUUUCACUCACCUGCCUUGUAAAAGGUUUUUA |
| | | CCCAUCAGACAUCGCUGUGGAAUGGGAGUCUAACGGACAGCCUGAAAACAA |
| | | CUAUAAAACCACUCCUCCUGUGCUGGACUCUGACGGAUCGUUCUUCUUGUA |
| | | CUCAAAACUAACCGUGGACAAGAGUAGAUGGCAGCAGGGAAAUGUUUUCUC |
| | | AUGCAGCGUCAUGCACGAGGCCCUGCAUAAUCACUACACCCAGAAGAGUUU |
| | | GUCUCUGAGCCCGGGUAAGGGAGGCGGCGGGGGCAUCGAAGGGCCUACUC |
| | | UGAGACAAUGGCUGGCAGCCCGGGCUGGCGGGGGUGGAGGGGGGGGAGG |
| | | CAUCGAGGGGCCAACCCUUCGCCAAUGGCUUGCGGCGCGAGCC |
| 76 | Sargramostim | AUGUGGCUGCAAUCCCUGCUGCUGCUUGGCACAGUGGCAUGCUCCAUCAGC |
| | | GCUCCUGCCCGAUCUCCCUCACCCAGCACACAACCCUGGGAGCAUGUCAAU |
| | | GCGAUACAGGAGGCCCUCCGGCUCUUAAAUCUCUCGCGCGAUACUGCAGCU |
| | | GAGAUGAACGAAACCGUGGAAGUGAUCAGUGAAAUGUUCGAUCUGCAGGAA |
| | | CCCACCUGUCUGCAGACCAGGCUAGAGUUGUACAAACAGGGCUUGAGAGGA |
| | | UCACUAACUAAACUUAAGGGGCCUCUGACGAUGAUGGCCUCCCACUAUAAG |
| | | CAGCACUGUCCACCAACUCCAGAAACAAGUUGCGCCACACAGAUCAUUACCU |
| | | UUGAGAGCUUUAAGGAGAACUUAAAAGACUUCCUCCUGGUUAUUCCUUUCG |
| | | ACUGCUGGGAGCCGGUACAGGAG |
| 77 | Anti-CD19 | AUGGGUGUCCCUACCCAGCUCCUGGGACUGCUCCUGCUGUGGAUCACCGA |
| | | CGCCAUCUGCGACAUCCAGAUGACCCAGAGCCCUGCCAGCCUGUCUACCAG |
| | | CCUGGGCGAGACAGUGACCAUCCAGUGUCAGGCCAGCGAGGACAUCUACUC |
| | | UGGCCUGGCUUGGUAUCAGCAGAAGCCCGGCAAGAGCCCUCAGCUGCUGAU |
| | | CUACGGCGCCAGCGACCUGCAGGACGGCGUGCCUAGCAGAUUCAGCGGCA |
| | | GCGGCUCCGGAACCCAGUACAGCCUGAAGAUCACCAGCAUGCAGACCGAGG |
| | | ACGAGGGCGUGUACUUCUGCCAGCAAGGCCUGACCUACCCUAGAACCUUCG |
| | | GAGGAGGCACCAAGCUGGAACUGAAGGGCGGAGGCGGAAGUGGAGGCGGA |
| | | GGAUCUGGCGGCGGAGGCUCUGAAGUGCAGCUGCAGCAGUCUGGCGCUGA |
| | | ACUGGUCCGGCCUGGCACUAGCGUGAAGCUGUCCUGCAAGGUGUCCGGCG |
| | | ACACCAUCACCUUCUACUACAUGCACUUCGUGAAGCAGAGGCCAGGACAGG |
| | | GCCUGGAAUGGAUCGGCAGAAUCGACCCUGAGGACGAGAGCACCAAGUACA |
| | | GCGAGAAGUUCAAGAACAAGGCCACCCUGACCGCCGACACCAGCAGCAACA |
| | | CCGCCUACCUGAAGCUGUCUAGCCUGACCUCCGAGGACACCGCCACCUACU |
| | | UUUGCAUCUACGGCGGCUACUACUUCGACUACUGGGGCCAGGGCGUGAUG |
| | | GUCACCGUGUCCAGCAUCGAGUUCAUGUACCCCCCUCCCUACCUGGACAAC |
| | | GAGAGAAGCAACGGCACCAUCAUCCACAUCAAAGAAAAGCACCUGUGCCACA |
| | | CCCAGAGCAGCCCCAAGCUGUUCUGGGCCCUGGUGGUGGUGGCCGGCGUG |
| | | CUGUUCUGUUACGGCCUGCUGGUCACAGUGGCCCUGUGCGUGAUCUGGAC |
| | | CAACAGCAGAAGAAACAGAGGCGGCCAGAGCGACUACAUGAACAUGACCCC |
| | | CAGAAGGCCAGGCCUGACCAGAAAGCCCUACCAGCCCUACGCCCCUGCCAG |
| | | AGACUUCGCCGCCUACAGACCCAGAGCCAAGUUCAGCAGAUCCGCCGAGAC |
| | | AGCCGCCAACCUGCAGGAUCCCAACCAGCUGUUCAACGAGCUGAACCUGGG |
| | | CAGACGGGAGGAAUUCGACGUGCUGGAAAAGAAGAGAGCCAGGGACCCCGA |
| | | GAUGGGCGGCAAGCAGCAGAGAAGAAGAAACCCUCAGGAAGGCGUCUACAA |
| | | CGCCCUGCAGAAAGACAAGAUGGCCGAGGCCUACAGCGAGAUCGGCACCAA |
| | | GGGCGAGAGAAGAAGGGGCAAGGGCCACGAUGGCCUGUUCCAGGGCCUGU |
| | | CCACCGCCACCAAGGACACCUUCGACGCCCUGCACAUGCAGACCCUGGCCC |
| | | CCAGA |

### Example 3. Nanoluciferase Study Design

This example describes an oral administration of recLemon LPMP/mRNA formulations comprising a lemon lipid reconstructed with C12-200 as the ionizable lipid, sterols (cholesterol), and PEG lipids (DMPE-PEG2k) that encapsulated mRNA. The recLemon LPMP/mRNA formulation tested in this example was prepared according to Example 2. The coding portion of the mRNA (nLuc-FLag and nLuc-secreted) used in this example are as follows:

nLuc-Flag:
N1-methylpseudouridine
Cap1 CleanCap AG
ORF Sequence:
(SEQ ID NO: 78)
ATGgtgttcacactcgaagatttcgttggggactggcgacaga
cagccggctacaacctggaccaagtccttgaacagggaggtgt
gtccagtttgtttcagaatctcggggtgtccgtaactccgatc
caaaggattgtcctgagcggtgaaaatgggctgaagatcgaca
tccatgtcatcatcccgtatgaaggtctgagcggcgaccaaat
gggccagatcgaaaaaatttttaaggtggtgtaccctgtggat
gatcatcactttaaggtgatcctgcactatggcacactggtaa
tcgacggggttacgccgaacatgatcgactatttcggacggcc
gtatgaaggcatcgccgtgttcgacggcaaaaagatcactgta
acagggaccctgtggaacggcaacaaaattatcgacgagcgcc
tgatcaaccccgacggctccctgctgttccgagtaaccatcaa
cggagtgaccggctggcggctgtgcgaacgcattctggcAgac
tacaaagaccatgacggtgattataaagatcatgacatcgatt
acaaggatgacgatgacaagTAA
nLuc-secreted:
N1-methylpseudouridine
Cap1 CleanCap AG
ORF Sequence:
(SEQ ID NO: 79)
atgaactccttctccacaagcgccttcggtccagttgccttct
ccctgggcctgctcctggtgttgcctgctgccttccctgcccc
agtcttcacactcgaagatttcgttggggactggcgacagaca
gccggctacaacctggaccaagtccttgaacagggaggtgtgt
ccagtttgtttcagaatctcggggtgtccgtaactccgatcca
aaggattgtcctgagcggtgaaaatgggctgaagatcgacatc
catgtcatcatcccgtatgaaggtctgagcggcgaccaaatgg
gccagatcgaaaaaatttttaaggtggtgtaccctgtggatga
tcatcactttaaggtgatcctgcactatggcacactggtaatc
gacggggttacgccgaacatgatcgactatttcggacggccgt
atgaaggcatcgccgtgttcgacggcaaaaagatcactgtaac
agggaccctgtggaacggcaacaaaattatcgacgagcgcctg
atcaaccccgacggctccctgctgttccgagtaaccatcaacg
gagtgaccggctggcggctgtgcgaacgcattctggcgtaa

In this example, in vivo level of nLuc-Flag and nLuc secreted and its effect on mice upon oral dosing of recLemon LPMP/mRNA formulation (200 ug/200 uL of nLuc mRNA) were measured. The treatment schedule is shown in the scheme below.

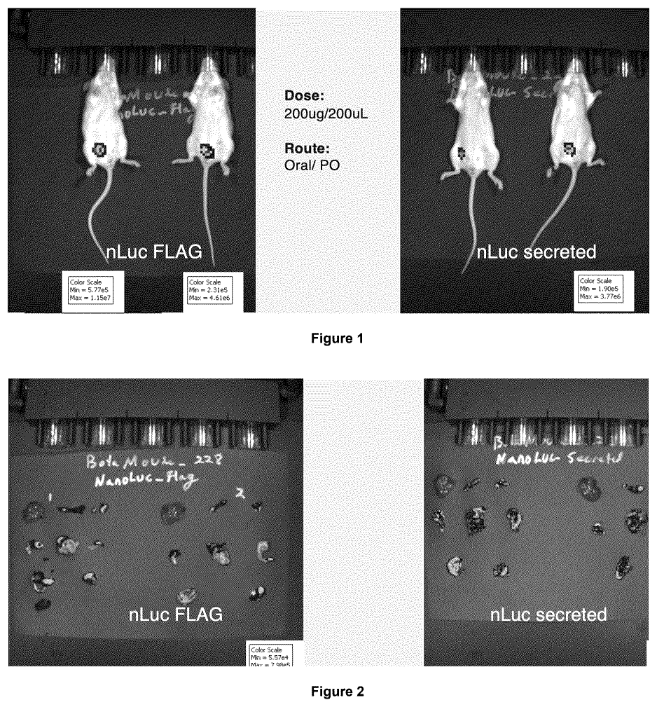

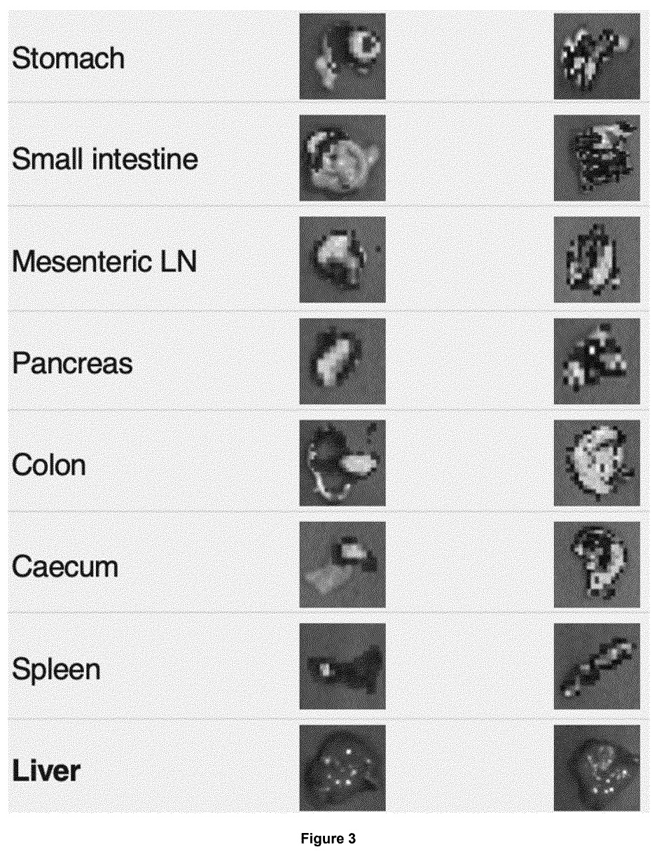

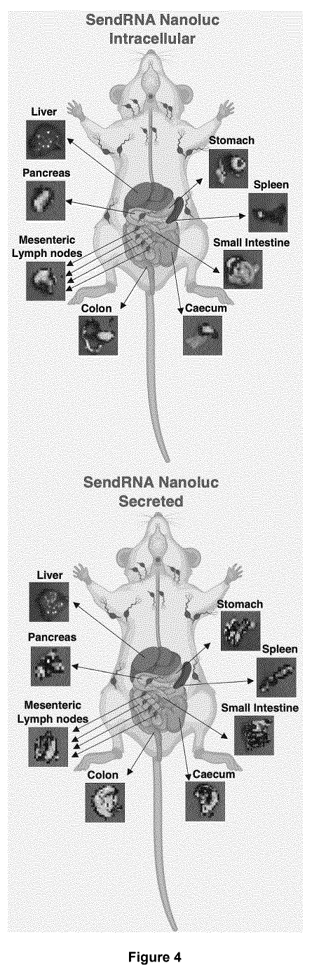

Mice received a P.O. (oral route) dose of 200 ug nanoLuc-Flag or nano-Luc secreted mRNA, and 6 hours later mice were dosed IP (intraperitoneal) with the substrate nano glow prior to whole body and organ IVIS in vivo bio-luminescence and fluorescence imaging via IVIS Spectrum (PerkinElmer). nLuc-Flag remained intracellular and was used to determine where transfection was occurring after oral delivery, and nLuc-secreted was used to track where secreted protein goes after oral delivery in addition to tracking transfected cells. The nLuc-secreted was also used to determine if oral delivery results in systemic delivery of secreted protein (liver expression indicates systemic delivery). The results are shown in FIGS. 1-4. FIGS. 1-3 are various photographs showing the level of nanoluciferase in mice treated orally with reconstructed LPMPs (recPMPs) derived from lemon, using C12-200 as ionizable lipid, and comprising nanoluciferase (nLuc) mRNA-nLuc-Flag (intracellular) and nLuc secreted in various organs in mice (e.g., liver, stomach, colon, spleen, small intestine, mesenteric lymph nodes, pancreas, and caecum) with 5 minutes exposure time. FIG. 4 provides further illustration of levels of nanoLuc-Flag (intracellular) or nano-Luc secreted in various organs in mice. These figures demonstrate that nLuc-Flag and nLuc-secreted were not detected in the liver (i.e., no systemic delivery), and they were only detected in the GI tract and associated lymphoid tissues. There was higher expression of nLuc-secreted in the colon compared to nLuc-Flag.

### Example 4. Administration of LNP/mRNA Formulations in Mice

If not specified, the LNP/mRNA formulation were prepared according to those described in Example 2.

### Formulation and Characterization of LNP/mRNA

This example further describes the formulation of several LNMPs formulated with novel ionizable lipids, structural lipids (either natural or synthetic), sterols, and PEG lipids, to encapsulate mRNA (e.g., nLuc-Flag mRNA, CRE recombinase mRNA) for LNP/mRNA formulation.

The coding portion of the nLuc-Flag mRNA used in this example is as follows:

nLuc-Flag:
N1-methylpseudouridine
Cap1 CleanCap AG
ORF Sequence:
(SEQ ID NO: 80)
ATGgtgttcacactcgaagatttcgttggggactggcgacagacagccggctacaacctggaccaagtccttgaacagggaggt
gtgtccagtttgtttcagaatctcggggtgtccgtaactccgatccaaaggattgtcctgagcggtgaaaatgggctgaagatcgaca
tccatgtcatcatcccgtatgaaggtctgagcggcgaccaaatgggccagatcgaaaaaatttttaaggtggtgtaccctgtggatg
atcatcactttaaggtgatcctgcactatggcacactggtaatcgacggggttacgccgaacatgatcgactatttcggacggccgt
atgaaggcatcgccgtgttcgacggcaaaaagatcactgtaacagggaccctgtggaacggcaacaaaattatcgacgagcgc
ctgatcaaccccgacggctccctgctgttccgagtaaccatcaacggagtgaccggctggcggctgtgcgaacgcattctggcAg
actacaaagaccatgacggtgattataaagatcatgacatcgattacaaggatgacgatgacaagTAA

Lipid nanoparticle (LNP) formulations were prepared according to Example 1-3, composed of ionizable lipid:structural lipid:sterol:PEG-lipid at given molar ratios outlined in Table 4. Lipids were solubilized in ethanol. These lipids were mixed at the indicated molar ratios and diluted in ethanol (organic phase) to 5.5 mM total lipid concentration and the mRNA solution (aqueous phase) were prepared with RNAse-free water and 100 mM citrate buffer pH 3 for a final concentration of 50 mM citrate buffer. Formulations were maintained at ionizable lipid to mRNA N:P ratio of 3:1-15:1 (Table 4).

TABLE 4
Exemplary mRNA-LNP formulations with novel ionizable lipids

| | Ionizable: | | | | | | |
|---|---|---|---|---|---|---|---|
| | Structural: | | | | | | |
| | Sterol: | | | | | | |
| | PEG-lipid | Ionizable | Structural | | | N:P | |
| Formulation | Molar ratio | lipid | lipid | Sterol | PEG-lipid | Ratio | Cargo |
| 2243 LNP | 50:10:38.5:1.5 | 2243 | DSPC | Chol | DMG- | 3:1 | nLuc-Flag |
| | | | | | PEG2k | | |
| 2243 LNP | 50:10:38.5:1.5 | 2243 | DSPC | Chol | DMG- | 3:1 | 2:1 nLuc-Flag: |
| | | | | | PEG2k | | CRE |

### Dosing and Collection in Mice

7-8 week old C57BL/6 or TdTomato mice were utilized to test the formulations provided in Table 4. Mice were obtained from Jackson Laboratories and allowed to acclimate for at least one week prior to manipulations.

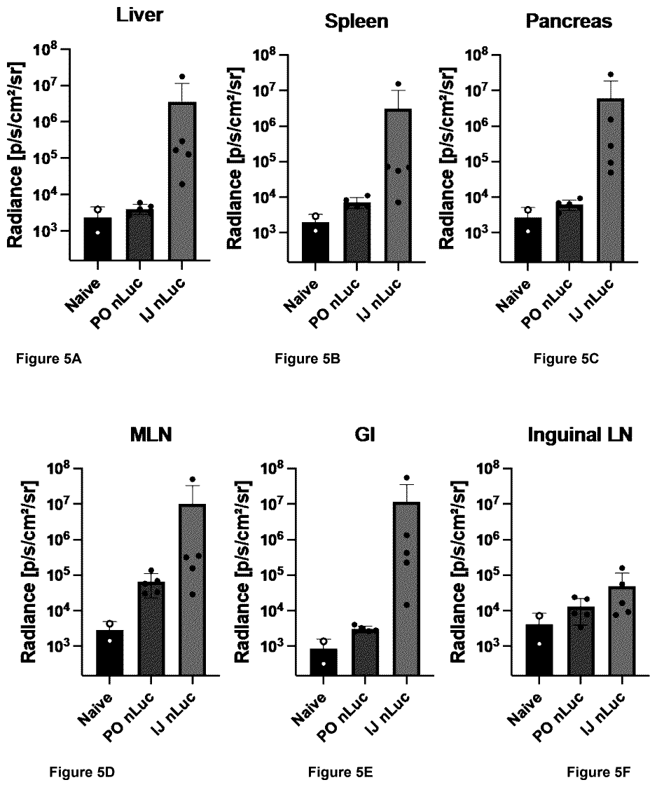

In one experiment using C57BL/6 mice, intrajejunal delivery and oral gavage (PO) delivery were compared using a lipid nanoparticle. FIGS. 5A-5F show the average radiance in the liver, spleen, pancreas, MLNs, gastro-intestine (GI), and inguinal lymph nodes of mice 24 hours after either oral (PO) or intrajejunal (IJ) delivery of 2243 LNP (nLuc-Flag mRNA, IJ-10 ug, PO-200 ug) (N=5/group), as compared to naïve mice (n=2). The results show that while IJ delivery route resulted in higher radiance in multiple locations, PO delivery route was sufficient to achieve the delivery of an mRNA composition and resulted in a high biodistribution to immune locations such as MLNs.

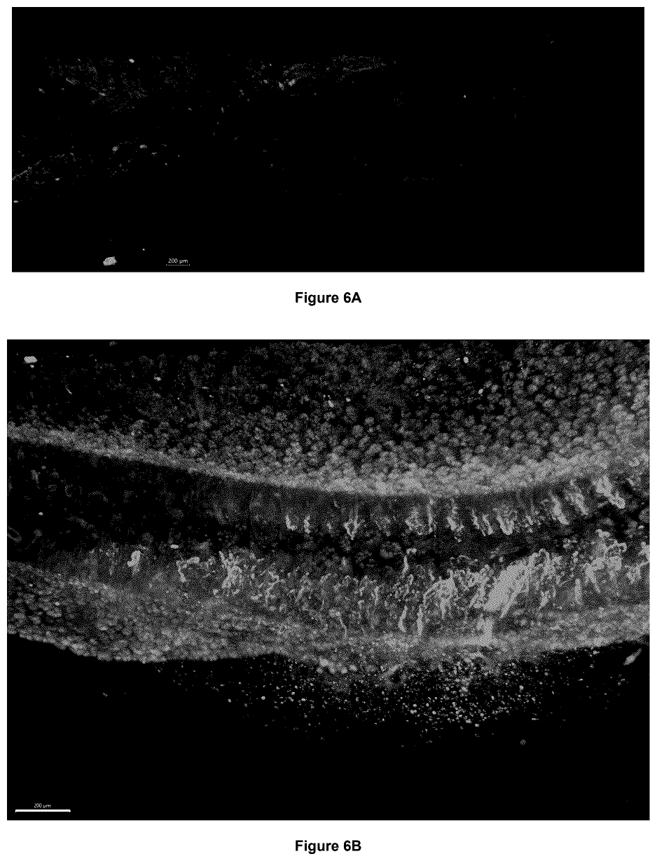

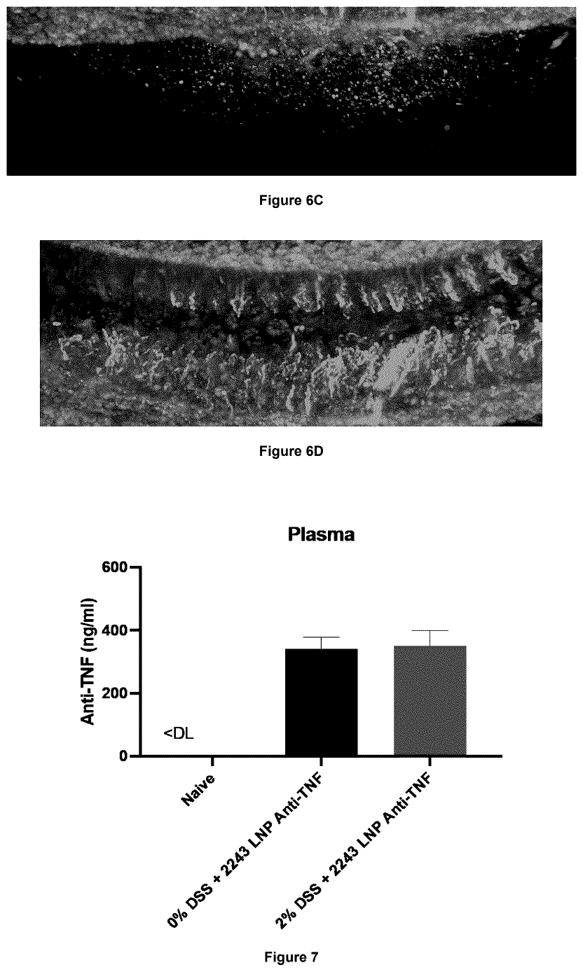

In a separate study, TdTomato mice were used to determine transfection of the GI tract. Briefly, two TdTomato mice were intrajejunally administered 2243 LNP (nLuc-Flag: CRE recombinase mRNA, 15 ug), and 48 hours later, the small intestine (SI) of the mice were imaged using IVIS. Regions that showed expression of nanoluciferase (i.e. were transfected by the particle) were further visualized via microscopy to identify TdTomato expression. The results are shown in FIGS. 6A-6D. FIG. 6A shows a section of SI from a naïve tdTomato mouse, indicating baseline levels. FIG. 6B shows a representative section of the SI from a mouse administered 2243 LNP (nLucFlag: CRE mRNA, 15 ug) intrajejunally 48 hours prior to collection. FIG. 6C highlights a Peyer's Patch previously shown in FIG. 6B. FIG. 6D highlights villi previously shown in FIG. 6B. These images indicate that a simulated oral delivery (e.g., IJ) of a lipid nanoparticle formulated with a novel ionizable lipid resulted in transfection of intestinal villi and cells within Peyer's patches in the small intestine.

### Example 5. Intravenous Delivery of a Polynucleotide Encoding an Antibody

If not specified, the formulations were prepared according to those described in Example 2.

The coding portion of the anti-TNF heavy chain mRNA is as follows:

(SEQ ID NO: 81)
auggaauuuggacugucuuggguuuuucugguagcucuguuucguggagugcaaugcgagguucagcuugucgagagcggcg
gcggucucgugcaacccggacggagucugagacucagcugugcugcuucaggauuuaccuucgacgauuaugcgaugcauugg
gugcggcaggcuccuggcaaaggauuagaaugggugagcgcaauuacauggaacucuggccacaucgacuacgccgauucagu
ggagggcagguucaccauuagcagagauaaugcuaagaauagcuuauacuugcagaugaacagccugcgcgccgaggauacu
gcgguguacuauugcgccaaggucaguuaucuguccaccgccagcucccuagauuauugggggcagggaaccuuggugacagu
caguuccgcaagcacgaaagggcccagcguuuucccucucgcacccucuucaaaauccacuucaggggggacagcugcacuggg
augucuugucaaggauuacuuccccgaaccugugaccgucucauggaauuccggugccuugaccucaggggugcauaccuucc
ccgcaguccuucagaguucuggacucuacucccugaguucugugguaacagucccuucuagcucccucgggacacagaccuaca
uuugcaaugugaaccauaaaccgaguaacacuaaaguggacaaaaaaguagaacccaaaagcugcgauaagacccacacgugc
cccccguguccggcccccgagcuucugggggcccuucgguguuccuguuuccaccuaagcccaaagacacacugaugaucucu
cgcacuccugaaguaacaugcgugguggucgacgugagccacgaagauccagaaguuaaguucaauugguauguugacgggg
uggaggugcacaacgccaagacuaagccaagggaggagcaauacaauuccacauaccgaguugucagcguacuuacgguucug
caccaagacuggcuaaaugguaaagaguacaaguguaaggugucuaacaaagcccugccagcgccgauagaaaagacuaucuc
aaaagcaaagggccagcccagagaaccucagguguacaccuugccaccaucaagggacgagcucaccaagaaucagguuaguc
ucaccugucuggugaagggguucuauccauccgacaucgccguggagugggagaguaacggccagcccgaaaacaauuacaag
acgacaccaccagucuuggauagcgacgguucuuucuuucuauauucgaagcucacugucgacaagucgcgguggcagcaggg
caacguauuuagcugcuccgucaugcaugaggcccugcacaaccacuauacucaaaaaucccuguccuuaucuccuggcaag

The coding portion of the anti-TNF light chain mRNA is as follows:

(SEQ ID NO: 82)
AUGGACAUGAGGGUACCAGCACAGUUGCUGGGACUGUUACUGCUCUGGCUCUCCGGUGCUC
GCUGUGAUAUCCAGAUGACACAGAGUCCCUCUAGCCUGAGCGCAUCAGUCGGAGACCGCGUG
ACAAUCACCUGCAGAGCUAGCCAGGGCAUAAGAAACUAUCUCGCAUGGUACCAGCAGAAACCC
GGAAAGGCCCCUAAGUUGCUGAUUUAUGCUGCUUCUACACUACAAAGCGGCGUGCCCAGCCG
AUUCUCCGGGUCUGGGAGUGGCACGGACUUUACUCUUACUAUCUCCUCACUGCAACCAGAGG
AUGUAGCCACGUACUAUUGUCAGCGUUACAAUCGGGCCCCCUACACAUUUGGCCAGGGAACC
AAAGUUGAAAUCAAAAGGACUGUGGCGGCCCCUUCGGUUUUCAUUUUCCCACCAUCAGACGA
GCAACUCAAGUCUGGUACAGCAUCCGUCGUCUGCCUUCUCAAUAACUUCUACCCGAGGGAGG
CCAAAGUCCAGUGGAAAGUUGACAAUGCCCUGCAGAGUGGGAACUCACAGGAAUCCGUGACC
GAACAAGACUCAAAGGAUAGCACCUAUAGUCUAAGCAGCACCUUGACUCUGUCGAAGGCCGAU
UAUGAAAAGCACAAAGUGUACGCGUGUGAGGUGACCCAUCAGGGCCUGUCCUCCCCUGUGAC
CAAGAGUUUUAACCGGGGGGAGUGCUGAUAAUAG

The coding portion of the anti-PCSK9 heavy chain mRNA (from Vernal) used in this example is as follows:

(SEQ ID NO: 83)
AUGGAGUUCGGUCUGUCUUGGGUCUUUCUUGUCGCUUUGUUUCGGGGAGUUCAGUGUGAAGUGCAACUGGUGC
AGUCAGGAGCCGAAGUGAAAAAGCCGGGUGCUAGCGUGAAAGUGUCCUGCAAAGCGUCGGGUUACACCCUGACUU
CCUACGGGAUUUCGUGGGUCCGCCAAGCCCCUGGCCAAGGACUGGAAUGGAUGGGCUGGGUGUCGUUCUAUAACG
GCAACACCAACUACGCGCAGAAGCUUCAGGGACGGGGGACUAUGACCACCGACCCGUCCACUUCCACCGCCUAUAUG
GAGUUGCGCAGCCUCCGGUCCGACGAUACUGCCGUGUACUACUGCGCAAGGGGUUACGGAAUGGACGUCUGGGGA
CAGGGUACUACCGUGACGGUGUCGUCAGCCAGCACCAAGGGCCCAUCCGUAUUCCCACUGGCCCCUUGCUCCCGCUC
CACCUCCGAAUCCACCGCCGCCCUGGGCUGCCUCGUGAAGGAUUACUUCCCCGAACCUGUGACCGUGUCCUGGAAU
UCAGGGGCCCUGACUAGCGGCGUGCACACUUUCCCGGCGGUGCUCCAGUCGAGCGGCCUGUACAGCCUCUCGAGUG
UCGUGACCGUGCCCUCCUCCAACUUCGGAACCCAGACUUACACAUGCAACGUCGACCACAAGCCUUCCAACACCAAG
GUCGACAAGACUGUGGAGAGAAAGUGCUGCGUGGAAUGCCCGCCAUGCCCUGCCCCUCCCGUGGCGGGACCGUCGG
UGUUCCUGUUCCCGCCAAAGCCGAAGGACACCCUGAUGAUCAGCAGAACUCCCGAAGUGACCUGUGUGGUGGUCGA
UGUGUCUCAUGAGGACCCCGAGGUGCAGUUCAAUUGGUACGUGGACGGGGUGGAGGUCCAUAACGCAAAGACCAA
GCCCCGCGAGGAACAGUUCAACUCCACCUUCCGGGUGGUGUCAGUGCUGACAGUCGUGCACCAAGAUUGGCUGAAC
GGAAAGGAGUACAAGUGCAAAGUGUCCAACAAGGGCCUGCCUGCCCCGAUUGAAAAGACCAUCUCCAAGACUAAGG
GCCAGCCGAGGGAGCCCCAGGUCUACACCCUGCCGCCCUCACGGGAAGAAAUGACUAAGAACCAAGUGUCCCUGACC
UGUCUGGUCAAGGGCUUUUACCCCUCGGACAUCGCCGUGGAGUGGGAGUCAAACGGACAGCCAGAGAACAACUACA
AGACCACCCCUCCUAUGCUGGACAGCGACGGAAGCUUCUUCCUGUACUCCAAGCUCACCGUGGAUAAGUCCAGAUG
GCAGCAGGGAAACGUGUUCUCGUGUUCCGUGAUGCACGAAGCUCUCCACAACCACUACACGCAAAAGUCCCUCAGC
CUGAGCCCGGGGAAAUAA

The coding portion of the anti-PCSK9 light chain mRNA (from Vernal) used in this example is as follows:

(SEQ ID NO: 84)
AUGAAGUACCUCUUGCCUACUGCUGCCGCCGGACUCUUGCUGCUUGCCGCCCAACCCGCUAUGGCCGAAUCGGCGC
UGACCCAGCCUGCCUCUGUCUCCGGUUCCCCGGGACAGUCAAUUACCAUCAGCUGCACUGGAACCUCCUCCGACGU
GGGCGGCUACAAUUCAGUGUCGUGGUACCAGCAGCACCCUGGGAAGGCACCCAAGCUGAUGAUCUACGAGGUGUC
CAACCGCCCAAGCGGAGUGUCAAACCGGUUCUCCGGAAGCAAAUCAGGAAACACCGCCUCCCUGACUAUCAGUGGGC
UGCAGGCCGAGGACGAAGCAGAUUACUACUGCAACUCGUACACCUCCACUAGCAUGGUGUUCGGCGGCGGGACUAA
GCUGACCGUGCUGGGACAGCCCAAGGCGGCCCCCUCCGUGACUCUGUUCCCGCCGUCGUCCGAGGAACUGCAGGCU
AACAAGGCCACCCUCGUGUGCCUGAUUAGCGACUUUUACCCUGGCGCCGUGACCGUGGCCUGGAAGGCGGAUUCG
UCGCCGGUCAAGGCCGGUGUCGAAACUACCACCCCGUCCAAGCAGUCCAACAACAAAUACGCCGCGAGCAGCUAUCU
GAGCCUCACCCCCGAACAAUGGAAGUCGCACCGCUCCUACUCCUGCCAAGUCACCCAUGAGGGUUCCACCGUGGAAA
AGACGGUGGCACCAACAGAGUGUUCCUAA

An alternative anti-PCSK9 heavy chain mRNA (from Aldevron) is as follows:

(SEQ ID NO: 85)
AUGGAAUUUGGACUGUCUUGGGUUUUUCUGGUAGCUCUGUUUCGUGGAGUGCAAUGCGAAGUCCAGCUCGUGCA
GUCUGGAGCAGAGGUCAAAAAACCAGGGGCUAGCGUGAAAGUUAGUUGCAAGGCUUCAGGGUACACGUUGACCUC
CUACGGCAUCUCCUGGGUGAGACAAGCACCCGGGCAAGGACUAGAGUGGAUGGGUUGGGUCAGUUUCUACAACGG
CAAUACCAACUACGCCCAGAAGCUUCAAGGGCGUGGUACAAUGACGACUGACCCAAGCACCAGCACGGCCUAUAUGG
AACUCAGAAGCCUUAGGUCUGACGACACAGCAGUUUACUAUUGUGCGCGAGGUUACGGCAUGGAUGUGUGGGGG
CAGGGGACCACAGUGACAGUGAGUUCCGCCAGCACUAAGGGGCCAAGCGUCUUUCCCCUGGCCCCGUGCUCAAGGU
CAACCAGUGAGAGUACCGCCGCCCUGGGCUGCUUAGUGAAAGAUUAUUUCCCCGAGCCAGUGACGGUGAGUUGGA
ACUCUGGCGCCCUCACAUCCGGAGUACACACUUUCCCGGCGGUGCUGCAGUCUAGUGGACUGUAUUCUUUAAGCA
GUGUCGUGACUGUGCCUUCUAGCAACUUCGGCACUCAGACAUACACAUGCAACGUUGACCACAAACCCUCCAAUAC
AAAGGUGGAUAAAACCGUUGAACGGAAAUGUUGUGUGGAAUGUCCACCGUGUCCAGCUCCACCAGUUGCUGGACC
UAGCGUCUUUUUGUUCCCUCCUAAGCCCAAAGAUACUCUGAUGAUCUCGCGGACCCCUGAGGUUACCUGCGUAGU
GGUAGACGUGUCACAUGAGGAUCCCGAAGUUCAGUUCAAUUGGUAUGUCGAUGGCGUAGAGGUCCACAACGCUAA
GACUAAGCCAAGGGAGGAACAGUUUAACUCUACAUUUCGGGUGGUUAGCGUCCUAACGGUGGUCCACCAGGAUUG
GCUGAAUGGAAAGGAGUACAAGUGCAAGGUGUCAAACAAGGGGCUCCCUGCACCUAUUGAAAAGACCAUCUCCAAG
ACUAAGGGUCAGCCACGCGAGCCCCAAGUAUACACCCUUCCUCCCUCCCGCGAAGAGAUGACCAAAAAUCAGGUGUC
ACUCACCUGCUUGGUCAAGGGCUUUUAUCCCUCCGACAUAGCAGUAGAAUGGGAGUCCAACGGACAGCCCGAAAAC
AAUUAUAAAACAACCCCGCCCAUGCUGGACUCAGACGGCUCUUUCUUCCUCUACUCGAAACUGACAGUGGACAAGU
CAAGAUGGCAACAGGGCAAUGUCUUUUCCUGUUCGGUGAUGCACGAGGCCCUGCAUAAUCAUUAUACUCAGAAAA
GCCUGUCCCUGAGCCCUGGAAAG

An alternative anti-PCSK9 light chain mRNA (from Aldevron) is as follows:

(SEQ ID NO: 86)
AUGAAGUAUCUACUGCCUACCGCUGCAGCUGGGCUGUUGCUUCUGGCUGCUCAACCAGCCAUGGCUGAGAGUGCU
CUGACCCAGCCAGCAAGUGUGAGCGGAAGCCCUGGCCAGUCAAUAACCAUCUCCUGUACUGGCACGUCAAGUGACG
UGGGGGGUUAUAAUAGCGUCUCAUGGUACCAGCAACACCCCGGGAAGGCGCCCAAGCUGAUGAUCUACGAAGUUA
GCAAUCGGCCGAGCGGAGUAUCUAAUAGGUUCUCGGGCUCAAAGUCUGGAAACACGGCAUCACUGACAAUUAGUG
GCCUCCAGGCAGAGGAUGAAGCUGACUACUAUUGUAACUCCUAUACUAGUACCAGUAUGGUGUUUGGCGGAGGGA
CCAAACUGACAGUCUUAGGGCAGCCUAAGGCCGCCCCCUCCGUCACACUGUUCCCACCUUCCAGCGAAGAACUACAA
GCAAACAAAGCUACAUUGGUCUGCCUCAUCUCCGACUUUUACCCGGGUGCCGUUACCGUGGCGUGGAAAGCCGAU
UCUAGCCCUGUGAAGGCCGGCGUAGAGACCACAACCCCAUCCAAGCAGAGCAACAAUAAAUACGCCGCCUCCUCAUA
CCUCUCGCUUACUCCCGAGCAAUGGAAAAGCCACAGAAGCUAUAGCUGCCAGGUUACUCAUGAGGGAUCUACUGUG
GAAAAGACAGUGGCUCCAACCGAGUGCUCC

For both heavy chain coding sequences, the encoded amino acid sequence is as follows:

(SEQ ID NO: 87)
MEFGLSWVFLVALFRGVQCEVQLVQSGAEVKKPGASVKVSCKASGYTLTS
YGISWVRQAPGQGLEWMGWVSFYNGNTNYAQKLQGRGTMTTDPSTSTAYM
ELRSLRSDDTAVYYCARGYGMDVWGQGTTVTVSSASTKGPSVFPLAPCSR
STSESTAALGCLVKDYFPEPVTVSWNSGALTSGVHTFPAVLQSSGLYSLS
SVVTVPSSNFGTQTYTCNVDHKPSNTKVDKTVERKCCVECPPCPAPPVAG
PSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTIS
KTKGQPREPQVYTLPPSREEMTKNQVSLTCLVKGFYPSDIAVEWESNGQP
ENNYKTTPPMLDSDGSFFLYSKLTVDKSRWQQGNVFSCSVMHEALHNHYT
QKSLSLSPGK

For both light chain coding sequences, the encoded amino acid sequence is as follows:

(SEQ ID NO: 88)
MKYLLPTAAAGLLLLAAQPAMAESALTQPASVSGSPGQSITISCTGTSSD
VGGYNSVSWYQQHPGKAPKLMIYEVSNRPSGVSNRFSGSKSGNTASLTIS
GLQAEDEADYYCNSYTSTSMVFGGGTKLTVLGQPKAAPSVTLFPPSSEEL
QANKATLVCLISDFYPGAVTVAWKADSSPVKAGVETTTPSKQSNNKYAAS
SYLSLTPEQWKSHRSYSCQVTHEGSTVEKTVAPTECS

### Formulation and Characterization

This example further describes the lemon LPMPs or LNPs formulated with ionizable lipids, sterols, and PEG lipids, to encapsulate an exemplary mRNA encoding an antibody for LPMP/mRNA formulations (e.g., PCSK9 (1:1 HC:LC) or for LNP/mRNA (e.g. TNF).

Lipid nanoparticle (LNP) formulation was prepared according to Example 2, composed of ionizable lipid (2243):structural lipid (DSPC):sterol (plant cholesterol):PEG-lipid (DMG-PEG2k) at the given molar ratios of 50:10:38.5:1.5. Lipids were solubilized in ethanol. These lipids were mixed at the indicated molar ratios and diluted in ethanol (organic phase) to 12.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1.

Exemplary lemon LPMP compositions (recLemon LPMP) were prepared according to Example 2, composed of ionizable lipid (2272): lemon lipids: cholesterol:DMG-PEG2k at given molar ratio of 35:20:42.5:2.5. Lipids were solubilized in ethanol. Formulations were then handled as above.

The lipid mix and mRNA solution (anti-PCSK9 antibody mRNA or anti-TNF alpha antibody mRNA) were mixed at a 1:3 ratio by volume, respectively, on the NANOASSEMBLR® IGNITE™ (Precision Nanosystems) at a total flow rate of 12 mL/minute. The resulting formulations were then loaded into Slide-A-Lyzer G2 dialysis cassettes (10 k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by tangential flow filtration (TFF) using Sartorius VIVAFLOW® 50R cassettes (100 k MWCO) at fixed feed flowrate of 100 ml/minute. The TFF-concentrated formulations were then further concentrated by centrifugation at 3000×g using AMICON® Ultra centrifugation filters (100 k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

Intravenous Antibody Dosing with LNP/mRNA Formulations

Initial experiments were carried out to determine the functionality of intravenous antibody dosing.

TABLE 5
Experimental Outline

| | Group | | Treat- | mRNA | | Dose |
|---|---|---|---|---|---|---|
| Group | Size | DSS | ment | cargo | ROA | Volume |
| 1 | 5 | 0% | — | — | — | — |
| 2 | 5 | 2% | — | — | — | — |
| 3 | 5 | 0% | LNP | Anti- | IV | 0.6 |
| | | | 2243 | TNF | | mg/kg |
| 4 | 5 | 2% | LNP | Anti- | IV | 0.6 |
| | | | 2243 | TNF | | mg/kg |

Bl6 mice were obtained from Jackson Laboratories and allowed to acclimate for one week prior to manipulations. Following acclimation, the mice were dosed according to Table 5. At Day 0, the mice were started on 0% or 2% dextran sulfate sodium (DSS) to induce intestinal inflammation, often used as a model of colitis. Blood and fecal samples were collected on Day 3. On Day 4, the appropriate experimental groups were intravenously dosed with 0.6 mg/kg of 2243 LNP/anti-TNF mRNA. Blood and fecal samples were collected on Day 5 and Day 7.

FIG. 7 depicts the concentration of anti-TNFα antibody levels in the plasma 24 h post-dose in mice given 0% DSS and a single intravenous dose of LNP/mRNA formulation (2243 LNP/anti-TNFα mRNA, 0.6 mg/kg) or mice given 2% DSS and a single intravenous dose of LNP/mRNA formulation (2243 LNP/anti-TNFα mRNA, 0.6 mg/kg). Control was plasma from naïve mice (0% DSS and no dose). N=5/group. The results indicate that anti-TNF was detectable in mice 24 hours after administration.

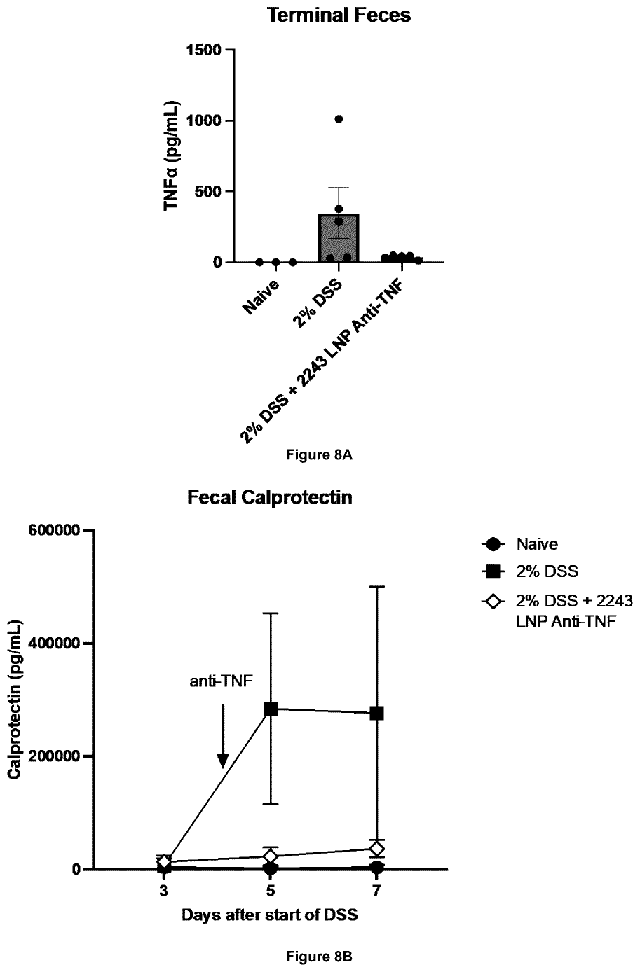

FIG. 8A shows the concentration of TNFα in feces three-days post dose in mice given 2% DSS or mice given 2% DSS and a single intravenous dose of LNP/mRNA formulation (2243 LNP/anti-TNFα mRNA, 0.6 mg/kg). Control was feces from naïve mice (0% DSS and no dose). N=5/group. FIG. 8B shows the concentration of calprotectin in feces pre-dose (3 days after the start of DSS), 24 h post-dose (5 days after start of DSS) in the mice given 2% DSS and in the mice given 2% DSS and a single intravenous dose of LNP/mRNA formulation (2243 LNP/anti-TNFα mRNA, 0.6 mg/kg), and 3 days post-dose (7 days after the start of DSS) in the mice given 2% DSS and in the mice given 2% DSS and a single intravenous dose of LNP/mRNA formulation (2243 LNP/anti-TNFα mRNA, 0.6 mg/kg). The control was feces from naïve mice (0% DSS and no administration of LNP 2243/anti-TNFα mRNA). N=5/group. The results indicate that a single intravenous dose of an LNP formulated with the ionizable lipid 2243, encapsulating anti-TNFα mRNA, reduced measures of inflammation in a colitis model, as measured through fecal TNF and calprotectin levels. The results of fecal calprotectin seen 24 h post-dose were sustained at 3 days post-dose.

Intravenous Antibody Dosing with LNMP/mRNA Formulations

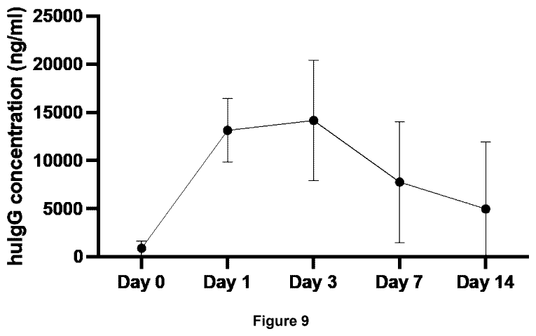

6-8 week old mice were obtained from Jackson Laboratories and allowed to acclimate for at least one week prior to manipulations. In one experiment, 100 μL of LPMP/mRNA formulation (recLemon LPMP 2272/anti-PCSK9 mRNA, 0.3 mg/kg) was dosed intravenously into the mice. Tail bleeds at Day 0, 1, 3, 7, and 14 were collected, processed and tested for anti-PCSK9 concentration. The results are shown in FIG. 9.

FIG. 9 shows the measured antibody concentration (huIgG, ng/mL) in the plasma of the mice at Days 0, 1, 3, 7, and 14 after a single intravenous dose of the LPMP/mRNA formulation employing the ionizable lipid 2272 (recLemon LPMP 2272/anti-PCSK9 mRNA, 0.3 mg/kg) N=5. The results indicate that the antibody was systemically detectable up to 14-days post-dose.

In a separate experiment, mice were intravenously dosed with a LPMP/mRNA formulation employing the ionizable lipid 2272 (recLemon LPMP 2272/anti-PCSK9 mRNA, 0.3 mg/kg), and 24 h post-dose, samples of plasma, colon and caecum, and mesenteric lymph nodes (MLNs) were collected. The concentration of anti-PCSK9 (ng/mL) was measured in the given samples. The results are shown in FIGS. 10A-10B.

FIG. 10A shows the measured antibody concentration (huIgG, ng/mL) in the plasma of the mice at 24 h after a single intravenous dose of the LPMP/mRNA formulation employing the ionizable lipid 2272 (recLemon LPMP 2272/anti-PCSK9 mRNA, 0.3 mg/kg) N=10. PBS was used as a control (N=1). FIG. 10B shows the measured antibody concentration (huIgG, ng/mL) in MLNs and colon/caecum of the mice at 24 h after a single intravenous dose of the LPMP/mRNA formulation employing the ionizable lipid 2272 (recLemon LPMP 2272/anti-PCSK9 mRNA, 0.3 mg/kg) N=10. PBS was used as a control (N=1). The results indicate that the antibody was detectable both systemically as well as in the lymph nodes and colon/caecum, as compared to the control mouse given PBS.

### Example 6. Administration of LNMP/mRNA Formulations in Mice

If not specified, the LNMP/mRNA formulation were prepared according to those described in Example 2.

### Formulation and Characterization of LNMP/mRNA

This example further describes the formulation of several LNMPs formulated with novel ionizable lipids, structural lipids (either natural or synthetic), sterols, and PEG lipids, to encapsulate mRNA (e.g., anti-PCSK9 mRNA) for LNMP/mRNA formulation. LNMP compositions where the natural lipids were extracted from a plant source, i.e., lemon in this example, were labeled as LPMP.

The coding portion of the anti-PCSK9 heavy chain mRNA (from Vernal) used in this example is as follows:

(SEQ ID NO: 83)
AUGGAGUUCGGUCUGUCUUGGGUCUUUCUUGUCGCUUUGUUUCGGGGAGUUCAGUGUGAAGUGCAACUGGUGC
AGUCAGGAGCCGAAGUGAAAAAGCCGGGUGCUAGCGUGAAAGUGUCCUGCAAAGCGUCGGGUUACACCCUGACUU
CCUACGGGAUUUCGUGGGUCCGCCAAGCCCCUGGCCAAGGACUGGAAUGGAUGGGCUGGGUGUCGUUCUAUAACG
GCAACACCAACUACGCGCAGAAGCUUCAGGGACGGGGGACUAUGACCACCGACCCGUCCACUUCCACCGCCUAUAUG
GAGUUGCGCAGCCUCCGGUCCGACGAUACUGCCGUGUACUACUGCGCAAGGGGUUACGGAAUGGACGUCUGGGGA
CAGGGUACUACCGUGACGGUGUCGUCAGCCAGCACCAAGGGCCCAUCCGUAUUCCCACUGGCCCCUUGCUCCCGCUC
CACCUCCGAAUCCACCGCCGCCCUGGGCUGCCUCGUGAAGGAUUACUUCCCCGAACCUGUGACCGUGUCCUGGAAU
UCAGGGGCCCUGACUAGCGGCGUGCACACUUUCCCGGCGGUGCUCCAGUCGAGCGGCCUGUACAGCCUCUCGAGUG
UCGUGACCGUGCCCUCCUCCAACUUCGGAACCCAGACUUACACAUGCAACGUCGACCACAAGCCUUCCAACACCAAG
GUCGACAAGACUGUGGAGAGAAAGUGCUGCGUGGAAUGCCCGCCAUGCCCUGCCCCUCCCGUGGCGGGACCGUCGG
UGUUCCUGUUCCCGCCAAAGCCGAAGGACACCCUGAUGAUCAGCAGAACUCCCGAAGUGACCUGUGUGGUGGUCGA
UGUGUCUCAUGAGGACCCCGAGGUGCAGUUCAAUUGGUACGUGGACGGGGUGGAGGUCCAUAACGCAAAGACCAA
GCCCCGCGAGGAACAGUUCAACUCCACCUUCCGGGUGGUGUCAGUGCUGACAGUCGUGCACCAAGAUUGGCUGAAC
GGAAAGGAGUACAAGUGCAAAGUGUCCAACAAGGGCCUGCCUGCCCCGAUUGAAAAGACCAUCUCCAAGACUAAGG
GCCAGCCGAGGGAGCCCCAGGUCUACACCCUGCCGCCCUCACGGGAAGAAAUGACUAAGAACCAAGUGUCCCUGACC
UGUCUGGUCAAGGGCUUUUACCCCUCGGACAUCGCCGUGGAGUGGGAGUCAAACGGACAGCCAGAGAACAACUACA
AGACCACCCCUCCUAUGCUGGACAGCGACGGAAGCUUCUUCCUGUACUCCAAGCUCACCGUGGAUAAGUCCAGAUG
GCAGCAGGGAAACGUGUUCUCGUGUUCCGUGAUGCACGAAGCUCUCCACAACCACUACACGCAAAAGUCCCUCAGC
CUGAGCCCGGGGAAAUAA

The coding portion of the anti-PCSK9 light chain mRNA (from Vernal) used in this example is as follows:

(SEQ ID NO: 84)
AUGAAGUACCUCUUGCCUACUGCUGCCGCCGGACUCUUGCUGCUUGCCGCCCAACCCGCUAUGGCCGAAUCGGCGC
UGACCCAGCCUGCCUCUGUCUCCGGUUCCCCGGGACAGUCAAUUACCAUCAGCUGCACUGGAACCUCCUCCGACGU
GGGCGGCUACAAUUCAGUGUCGUGGUACCAGCAGCACCCUGGGAAGGCACCCAAGCUGAUGAUCUACGAGGUGUC
CAACCGCCCAAGCGGAGUGUCAAACCGGUUCUCCGGAAGCAAAUCAGGAAACACCGCCUCCCUGACUAUCAGUGGGC
UGCAGGCCGAGGACGAAGCAGAUUACUACUGCAACUCGUACACCUCCACUAGCAUGGUGUUCGGCGGCGGGACUAA
GCUGACCGUGCUGGGACAGCCCAAGGCGGCCCCCUCCGUGACUCUGUUCCCGCCGUCGUCCGAGGAACUGCAGGCU
AACAAGGCCACCCUCGUGUGCCUGAUUAGCGACUUUUACCCUGGCGCCGUGACCGUGGCCUGGAAGGCGGAUUCG
UCGCCGGUCAAGGCCGGUGUCGAAACUACCACCCCGUCCAAGCAGUCCAACAACAAAUACGCCGCGAGCAGCUAUCU
GAGCCUCACCCCCGAACAAUGGAAGUCGCACCGCUCCUACUCCUGCCAAGUCACCCAUGAGGGUUCCACCGUGGAAA
AGACGGUGGCACCAACAGAGUGUUCCUAA

An alternative anti-PCSK9 heavy chain mRNA (from Aldevron) is as follows:

(SEQ ID NO: 85)
AUGGAAUUUGGACUGUCUUGGGUUUUUCUGGUAGCUCUGUUUCGUGGAGUGCAAUGCGAAGUCCAGCUCGUGCA
GUCUGGAGCAGAGGUCAAAAAACCAGGGGCUAGCGUGAAAGUUAGUUGCAAGGCUUCAGGGUACACGUUGACCUC
CUACGGCAUCUCCUGGGUGAGACAAGCACCCGGGCAAGGACUAGAGUGGAUGGGUUGGGUCAGUUUCUACAACGG
CAAUACCAACUACGCCCAGAAGCUUCAAGGGCGUGGUACAAUGACGACUGACCCAAGCACCAGCACGGCCUAUAUGG
AACUCAGAAGCCUUAGGUCUGACGACACAGCAGUUUACUAUUGUGCGCGAGGUUACGGCAUGGAUGUGUGGGGG
CAGGGGACCACAGUGACAGUGAGUUCCGCCAGCACUAAGGGGCCAAGCGUCUUUCCCCUGGCCCCGUGCUCAAGGU
CAACCAGUGAGAGUACCGCCGCCCUGGGCUGCUUAGUGAAAGAUUAUUUCCCCGAGCCAGUGACGGUGAGUUGGA
ACUCUGGCGCCCUCACAUCCGGAGUACACACUUUCCCGGCGGUGCUGCAGUCUAGUGGACUGUAUUCUUUAAGCA
GUGUCGUGACUGUGCCUUCUAGCAACUUCGGCACUCAGACAUACACAUGCAACGUUGACCACAAACCCUCCAAUAC
AAAGGUGGAUAAAACCGUUGAACGGAAAUGUUGUGUGGAAUGUCCACCGUGUCCAGCUCCACCAGUUGCUGGACC
UAGCGUCUUUUUGUUCCCUCCUAAGCCCAAAGAUACUCUGAUGAUCUCGCGGACCCCUGAGGUUACCUGCGUAGU
GGUAGACGUGUCACAUGAGGAUCCCGAAGUUCAGUUCAAUUGGUAUGUCGAUGGCGUAGAGGUCCACAACGCUAA
GACUAAGCCAAGGGAGGAACAGUUUAACUCUACAUUUCGGGUGGUUAGCGUCCUAACGGUGGUCCACCAGGAUUG
GCUGAAUGGAAAGGAGUACAAGUGCAAGGUGUCAAACAAGGGGCUCCCUGCACCUAUUGAAAAGACCAUCUCCAAG
ACUAAGGGUCAGCCACGCGAGCCCCAAGUAUACACCCUUCCUCCCUCCCGCGAAGAGAUGACCAAAAAUCAGGUGUC
ACUCACCUGCUUGGUCAAGGGCUUUUAUCCCUCCGACAUAGCAGUAGAAUGGGAGUCCAACGGACAGCCCGAAAAC
AAUUAUAAAACAACCCCGCCCAUGCUGGACUCAGACGGCUCUUUCUUCCUCUACUCGAAACUGACAGUGGACAAGU
CAAGAUGGCAACAGGGCAAUGUCUUUUCCUGUUCGGUGAUGCACGAGGCCCUGCAUAAUCAUUAUACUCAGAAAA
GCCUGUCCCUGAGCCCUGGAAAG

An alternative anti-PCSK9 light chain mRNA (from Aldevron) is as follows:

(SEQ ID NO: 86)
AUGAAGUAUCUACUGCCUACCGCUGCAGCUGGGCUGUUGCUUCUGGCUGCUCAACCAGCCAUGGCUGAGAGUGCU
CUGACCCAGCCAGCAAGUGUGAGCGGAAGCCCUGGCCAGUCAAUAACCAUCUCCUGUACUGGCACGUCAAGUGACG
UGGGGGGUUAUAAUAGCGUCUCAUGGUACCAGCAACACCCCGGGAAGGCGCCCAAGCUGAUGAUCUACGAAGUUA
GCAAUCGGCCGAGCGGAGUAUCUAAUAGGUUCUCGGGCUCAAAGUCUGGAAACACGGCAUCACUGACAAUUAGUG
GCCUCCAGGCAGAGGAUGAAGCUGACUACUAUUGUAACUCCUAUACUAGUACCAGUAUGGUGUUUGGCGGAGGGA
CCAAACUGACAGUCUUAGGGCAGCCUAAGGCCGCCCCCUCCGUCACACUGUUCCCACCUUCCAGCGAAGAACUACAA
GCAAACAAAGCUACAUUGGUCUGCCUCAUCUCCGACUUUUACCCGGGUGCCGUUACCGUGGCGUGGAAAGCCGAU
UCUAGCCCUGUGAAGGCCGGCGUAGAGACCACAACCCCAUCCAAGCAGAGCAACAAUAAAUACGCCGCCUCCUCAUA
CCUCUCGCUUACUCCCGAGCAAUGGAAAAGCCACAGAAGCUAUAGCUGCCAGGUUACUCAUGAGGGAUCUACUGUG
GAAAAGACAGUGGCUCCAACCGAGUGCUCC

For both heavy chain coding sequences, the encoded amino acid sequence is as follows:

(SEQ ID NO: 87)
MEFGLSWVFLVALFRGVQCEVQLVQSGAEVKKPGASVKVSCKASGYTLTS
YGISWVRQAPGQGLEWMGWVSFYNGNTNYAQKLQGRGTMTTDPSTSTAYM
ELRSLRSDDTAVYYCARGYGMDVWGQGTTVTVSSASTKGPSVFPLAPCSR
STSESTAALGCLVKDYFPEPVTVSWNSGALTSGVHTFPAVLQSSGLYSLS
SVVTVPSSNFGTQTYTCNVDHKPSNTKVDKTVERKCCVECPPCPAPPVAG
PSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTIS
KTKGQPREPQVYTLPPSREEMTKNQVSLTCLVKGFYPSDIAVEWESNGQP
ENNYKTTPPMLDSDGSFFLYSKLTVDKSRWQQGNVFSCSVMHEALHNHYT
QKSLSLSPGK

For both light chain coding sequences, the encoded amino acid sequence is as follows:

(SEQ ID NO: 88)
MKYLLPTAAAGLLLLAAQPAMAESALTQPASVSGSPGQSITISCTGTSSD
VGGYNSVSWYQQHPGKAPKLMIYEVSNRPSGVSNRFSGSKSGNTASLTIS
GLQAEDEADYYCNSYTSTSMVFGGGTKLTVLGQPKAAPSVTLFPPSSEEL
QANKATLVCLISDFYPGAVTVAWKADSSPVKAGVETTTPSKQSNNKYAAS
SYLSLTPEQWKSHRSYSCQVTHEGSTVEKTVAPTECS

Exemplary lemon LPMP compositions were formulated according to Example 1-3, composed of ionizable lipid:lemon lipid:sterol:PEG-lipid at given molar ratios outlined in Table 6. Lipids were solubilized in ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 6).

The lipid mix and mRNA solution were mixed at a 1:3 ratio by volume, respectively, on the NANOASSEMBLR® IGNITE™ (Precision Nanosystems) at a total flow rate of 14 mL/minute. The resulting formulations were then loaded into Slide-A-Lyzer G2 dialysis cassettes (10 k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by centrifugation at 2000×g using AMICON® Ultra centrifugation filters (100 k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

TABLE 6
LPMP formulations with novel ionizable lipids

| | Ionizable: | | | | | | |
|---|---|---|---|---|---|---|---|
| | Structural: | | | | | | |
| | Sterol: | | | | | | |
| | PEG-lipid | Ionizable | Structural | | | | |
| Formulation | Molar ratio | lipid | lipid | Sterol | PEG-lipid | N:P Ratio | Cargo |
| 2272 LPMP | 35:20:42.5:2.5 | 2272 | Naturally | Chol | DMG- | 6:1 | 1:1 HC:LC* |
| | | | derived | | PEG2k | | anti-PCSK9 |
| | | | lemon lipid | | | | |

*1:1 HC:LC means 1:1 heavy chain : light chain

### Dosing and Collection in Mice

7-8 week old C57BL/6 mice were utilized to test the formulations provided in Table 6, with PBS acting as control. Mice were obtained from Jackson Laboratories and allowed to acclimate for at least one week prior to manipulations.

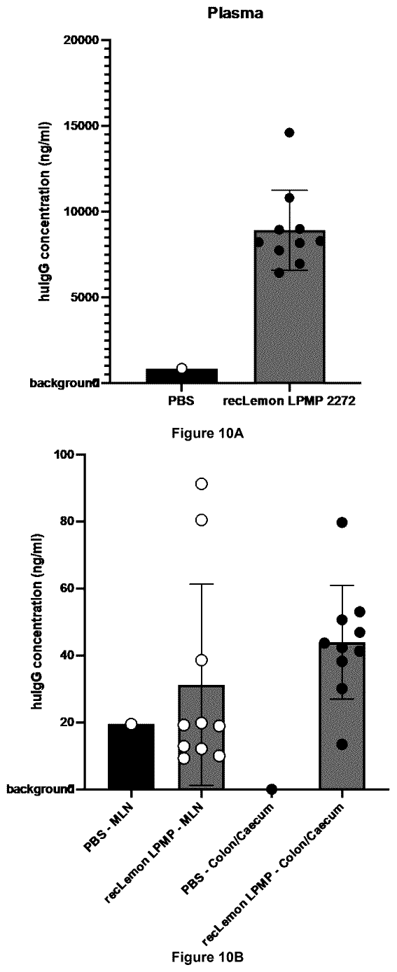

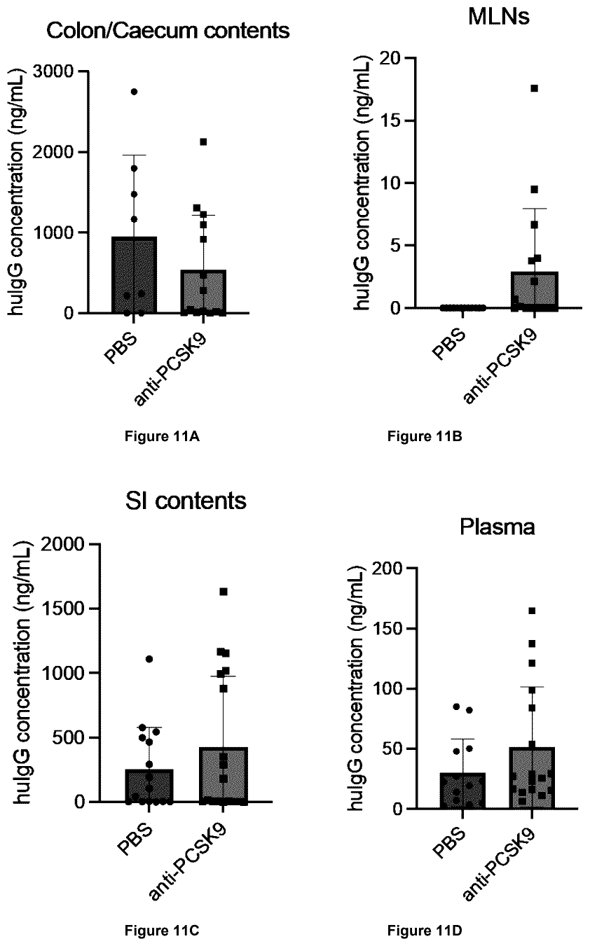

In one experiment, antibody concentration was measured using intrajejunal dosing of 19 mice over the course of 3 independent experiments. FIGS. 11A-11D show the antibody concentration (huIgG, ng/mL) in the colon/caecum contents, mesenteric lymph nodes (MLNs), small intestine (SI) content, and plasma of mice 24 hours after intrajejunal delivery of 2272 LPMP (anti-PCSK9 mRNA, 65-75 ug). N=19/3 independent experiments as compared to PBS (n=16/3 independent experiments). Statistically identified outliers were removed. The results show that intrajejunal delivery, an alternative model of oral delivery, was sufficient to deliver an mRNA therapeutic composition to mice.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. Other embodiments are within the claims.

1. A method for delivering an RNA composition to a subject for in vivo production of a protein or a peptide in the subject, comprising:
administering to the subject an RNA composition comprising a polynucleotide that encodes a protein or a peptide, formulated within
(a) a plurality of lipid nanoparticles (LNP) comprising synthetic structural lipids and an ionizable lipid, or
(b) a lipid reconstructed natural messenger packs (LNMPs) comprising natural lipids and an ionizable lipid,
wherein the ionizable lipid has two or more of the characteristics listed below:
(i) at least one ionizable amine functional groups;
(ii) at least three lipid tails, wherein each of the lipid tails is at least six carbon atoms in length;
(iii) a pKa of about 4.5 to about 7.5;
(iv) an ionizable amine functional group and a heterorganic functional group separated by a chain of at least two atoms; and
(v) an N:P ratio of at least 3.

2. The method of claim 1, wherein the RNA composition is administered orally, enterally, or systemically.

3. (canceled)

4. The method of claim 1, wherein the polynucleotide is an mRNA or circRNA, and optionally wherein the mRNA or circRNA is derived from
(a) a DNA molecule; or
(b) an RNA molecule, wherein T is substituted with U.

5. The method of claim 1, wherein the protein or peptide comprises an antibody, and optionally wherein the antibody is a therapeutic.

6. The method of claim 5, wherein the antibody is, a TNF inhibitor or PCSK9 inhibitor.

7. The method of claim 1, wherein the in vivo production of the protein or the peptide occurs in the subject's stomach, small intestine, mesenteric lymph node, pancreas, colon, caecum, and/or spleen.

8. The method of claim 1, wherein the administering of the RNA composition results in expression of the protein or the peptide encoded by the polynucleotide detectable in one or more organs in the subject at least about 6 hours after administration, and
wherein the organs are present along the transit route of the digestive track, and the RNA composition accesses the organs through the lymphatic transport system.

9. The method of claim 1, wherein the protein or the peptide encoded by the polynucleotide is detectable in the subject's mesenteric lymph node, pancreas, stomach, colon, small intestine, spleen, villi, and/or Peyer's patches at least about 6 hours after administration.

10. The method of claim 1, wherein the protein or the peptide encoded by the polynucleotide is not detectable in the subject's liver at least about 6 hours after administration.

11. A method of treating a disease or disorder associated with the gastrointestinal tract, stomach, small or large intestine, mesenteric lymph node, pancreas, colon or rectum, caecum, and/or spleen, comprising:
orally or enterally administering an RNA composition having a polynucleotide encoding one or more polypeptides formulated within
(a) a plurality of lipid nanoparticles (LNP) comprising synthetic structural lipids and an ionizable lipid, or
(b) a lipid reconstructed natural messenger packs (LNMPs) comprising natural lipids and an ionizable lipid,
wherein the ionizable lipid has two or more of the characteristics listed below:
(i) at least one ionizable amine functional group;
(ii) at least three lipid tails, wherein each of the lipid tails is at least six carbon atoms in length;
(iii) a pKa of about 4.5 to about 7.5;
(iv) an ionizable amine functional group and a heterorganic functional group separated by a chain of at least two atoms; and
(v) an N:P ratio of at least 3.

12. The method of claim 11, wherein the disease or disorder is pancreatitis, IBD, Crohn's disease, colorectal cancer, or ulcerative colitis.

13. The method of claim 1, wherein the RNA composition is formulated within (a) the plurality of lipid nanoparticles (LNP) comprising synthetic structural lipids and the ionizable lipid, wherein the synthetic structural lipid of the LNP composition is a phospholipid selected from the group consisting of lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof.

14. The method of claim 1, wherein the RNA composition is formulated within (b) the lipid reconstructed natural messenger pack (LNMP) comprising natural lipids and the ionizable lipid.

15-20. (canceled)

21. The method of claim 1, wherein the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

22. The method of claim 1, wherein the ionizable lipid is selected from one of the following groups of compounds:
i) a compound of formula 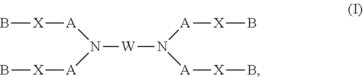
a pharmaceutically acceptable salt thereof, or a stereoisomer of any of the foregoing, wherein:
each A is independently C₁-C₁₆ branched or unbranched alkyl or C₁-C₁₆ branched or unbranched alkenyl, optionally substituted with heteroatom or substituted with OH, SH, or halogen;
each B is independently C₁-C₁₆ branched or unbranched alkyl or C₁-C₁₆ branched or unbranched alkenyl, optionally substituted with heteroatom or substituted with OH, SH, or halogen;
each X is independently a biodegradable moiety; and
W is 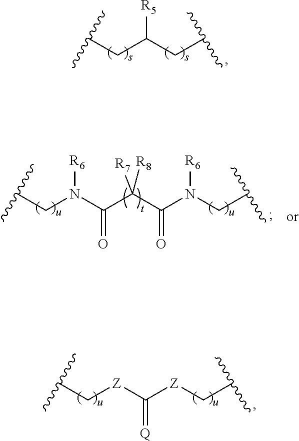
wherein
R₅ is OH, SH, or NR₁₀R₁₁;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, or NR₁₀R₁₁, wherein each R₁₀ and R₁₁ is independently H, C₁-C₃ alkyl, or R₁₀ and R₁ are taken together to form a heterocyclic ring;
each s is independently 1, 2, 3, 4, or 5;
each u is independently 1, 2, 3, 4, or 5;
t is 1, 2, 3, 4 or 5;
each Z is independently absent, O, S, or NR₁₂, wherein R₁₂ is H, C₁-C₇ branched or unbranched alkyl, or C₂-C₇ branched or unbranched alkenyl, and
Q is O, S, or NR₁₃, wherein each R₁₃ is H, or C₁-C₅ alkyl;
ii) a compound of formula 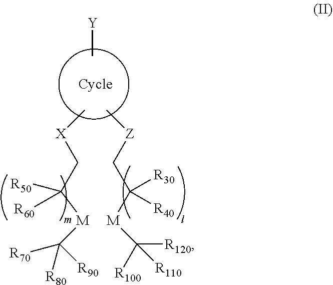
a pharmaceutically acceptable salt thereof, or a stereoisomer of any of the foregoing, wherein: 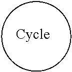
is a cyclic or heterocyclic moiety;
Y is alkyl, hydroxy, hydroxyalkyl or 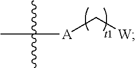
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
iii) a compound of formula 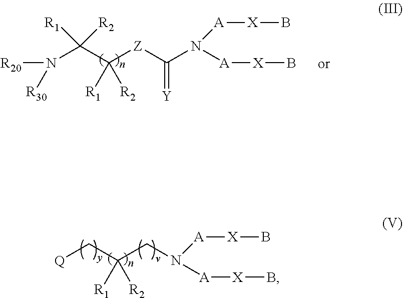
pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or R₁₀ and R₁₁ are taken together to form a heterocyclic ring;
n is 0, 1, 2, 3 or 4;
Y is O or S;
Z is absent, O, S, or N(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
v is 0, 1, 2, 3, or 4;
y is 0, 1, 2, 3, or 4;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen; and
each X is independently a biodegradable moiety; and
iv) a lipid comprising at least one head group and at least one tail group of formula (TI) or (TI′) 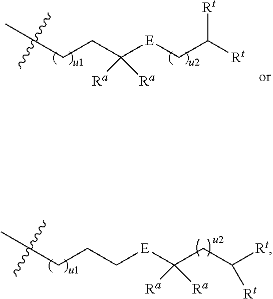
a pharmaceutically acceptable salt thereof, or a stereoisomer of any of the foregoing,
wherein:
E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl;
R₁₃ is branched or unbranched C₃-C₁₀ alkyl;
r is 1, 2, 3, 4, or 5;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl; 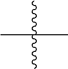
represents the bond connecting the tail group to the head group; and
wherein the lipid has a pKa from about 4 to about 8.

23-24. (canceled)

25. The method of claim 22, wherein the natural lipids of the LNMP are extracted from lemon or algae.

26. The method of claim 1, wherein the LNMP or the LNP composition further comprise a sterol and a polyethylene glycol (PEG)-lipid conjugate, and wherein: the LNMP comprises:
about 20 mol % to about 50 mol % of the ionizable lipid,
about 5 mol % to about 60 mol % of the natural lipids,
about 7 mol % to about 50 mol % of the sterol, and
about 0.5 mol % to about 3 mol % of the polyethylene glycol (PEG)-lipid conjugate: or wherein the LNP composition comprises:
about 20 mol % to about 50 mol % of the ionizable lipid,
about 5 mol % to about 60 mol % of the synthetic structural lipids,
about 7 mol % to about 50 mol % of the sterol, and
about 0.5 mol % to about 3 mol % of the polyethylene glycol (PEG)-lipid conjugate.

27-29. (canceled)

30. The method of claim 26, wherein the LNMP or the LNP composition comprises ionizable lipid:natural lipids:sterol:PEG-lipid at a molar ratio of about 35:50:12.5:2.5, about 35:20:42.5:2.5, about 35:30:32.5:2.5, about 35:16:46.5:2.5, about 35:25:37.5:2.5, about 35:40:22.5:2.5, about 45:10:43.5:1.5, about 50:20:28.5:1.5, or about 50:10:38.5:1.5.

31-34. (canceled)

35. The method of claim 1, wherein the RNA composition has a total lipid:polynucleotide weight ratio ranging from about 40:1 to about 28:1.

36. (canceled)

37. An oral vaccination composition, comprising an RNA composition comprising a polynucleotide encoding one or more polypeptides formulated within
(a) a plurality of lipid nanoparticles (LNP) comprising synthetic structural lipids and comprising an ionizable lipid, or
(b) a lipid reconstructed natural messenger packs (LNMPs) comprising natural lipids and an ionizable lipid,
wherein the ionizable lipid has two or more of the characteristics listed below:
(i) at least one ionizable functional group amine;
(ii) at least three lipid tails, wherein each of the lipid tails is at least six carbon atoms in length;
(iii) a pKa of about 4.5 to about 7.5;
(iv) an ionizable amine functional group and a heterorganic functional group separated by a chain of at least two atoms; and
(v) an N:P ratio of at least 3, wherein the RNA composition is formulated in an oral dosage form.

38-39. (canceled)
